(19) United States
(12) Patent Application Publication
(10) Pub. No.: US 2024/0024488 A1
(43) Pub. Date: Jan. 25, 2024

(54) COMPOUNDS AND USES THEREOF

(71) Applicant: Foghorn Therapeutics Inc., Indianapolis, IN (US)

(72) Inventors: Matthew NETHERTON, Cambridge, MA (US); Francois BRUCELLE, Belmont, MA (US); Jing DENG, Cambridge, MA (US); Johannes H. VOIGT, Cambridge, MA (US)

(21) Appl. No.: 18/253,541

(22) PCT Filed: Nov. 22, 2021
(86) PCT No.: PCT/US2021/060403

§ 371 Date: May 18, 2023

Related U.S. Application Data
(60) Provisional application No. 63/116,484, filed on Nov. 20, 2020.

(51) Int. Cl. A61K 47/54; A61K 47/55

(52) U.S. Cl. CPC A61K 47/545; A61K 47/55

(57) ABSTRACT

The present disclosure features compounds useful for the treatment of BAF complex-related disorders.

### BACKGROUND

The invention relates to compounds useful for modulating BRG1- or BRM-associated factors (BAF) complexes. In particular, the invention relates to compounds useful for treatment of disorders associated with BAF complex function.

Chromatin regulation is essential for gene expression, and ATP-dependent chromatin remodeling is a mechanism by which such gene expression occurs. The human Switch/Sucrose Non-Fermentable (SWI/SNF) chromatin remodeling complex, also known as BAF complex, has two SWI2-like ATPases known as BRG1 (Brahma-related gene-1) and BRM (Brahma). The transcription activator BRG1, also known as ATP-dependent chromatin remodeler SMARCA4, is encoded by the SMARCA4 gene on chromosome 19. BRG1 is overexpressed in some cancer tumors and is needed for cancer cell proliferation. BRM, also known as probable global transcription activator SNF2L2 and/or ATP-dependent chromatin remodeler SMARCA2, is encoded by the SMARCA2 gene on chromosome 9 and has been shown to be essential for tumor cell growth in cells characterized by loss of BRG1 function mutations. Deactivation of BRG and/or BRM results in downstream effects in cells, including cell cycle arrest and tumor suppression.

### SUMMARY

The present invention features compounds useful for modulating a BAF complex. In some embodiments, the compounds are useful for the treatment of disorders associated with an alteration in a BAF complex, e.g., a disorder associated with an alteration in one or both of the BRG1 and BRM proteins. The compounds of the invention, alone or in combination with other pharmaceutically active agents, can be used for treating such disorders.

In an aspect, the invention features a compound, or a pharmaceutically acceptable salt thereof, having the structure of Formula I:

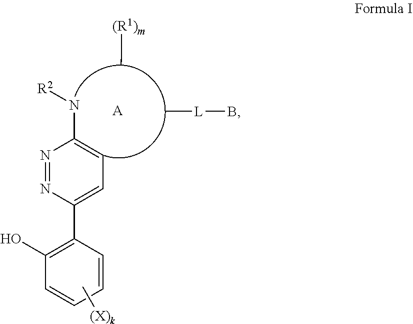

where
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, or optionally substituted C₃-C₈ cycloalkyl (e.g., each R¹ is, independently, halo or optionally substituted C₁-C₆ alkyl);
R² is H or optionally substituted C₁-C₅ alkyl;
each X is, independently, halo;
L is a linker; and
B is a degradation moiety.

In some embodiments, the compound has the structure of Formula I-A:

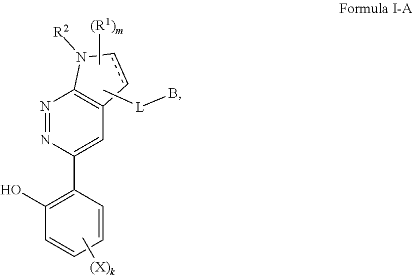

where the dashed bond represents a single or double bond.

In some embodiments, the compound has the structure of Formula I-B:

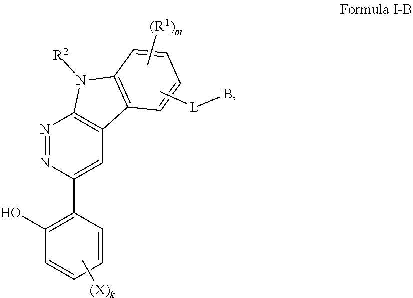

In some embodiments, the compound has the structure of Formula I-C:

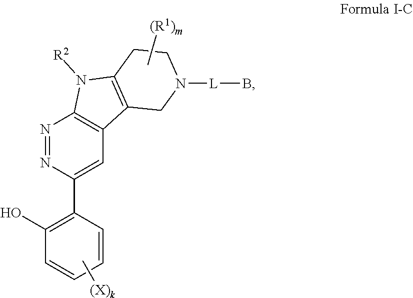

where each R¹ is, independently, optionally substituted C₁-C₆ alkyl.

In some embodiments, the compound has the structure of Formula I-D:

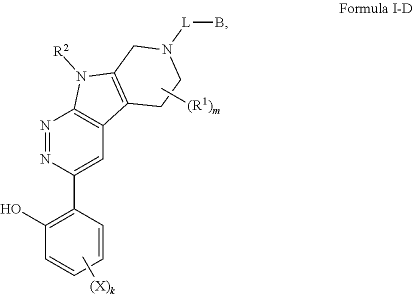

where each R¹ is, independently, optionally substituted C₁-C₆ alkyl.

In some embodiments, the compound has the structure of Formula I-E:

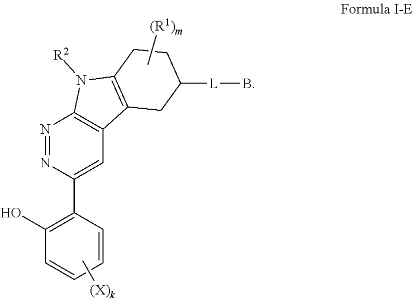

In some embodiments, the compound has the structure of Formula I-F:

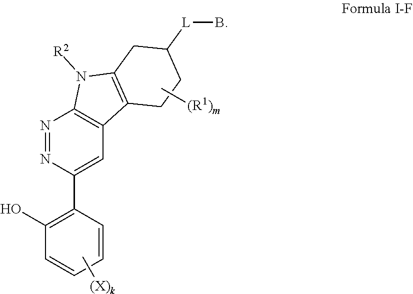

In some embodiments, R² is hydrogen. In some embodiments, m is 0.

In some embodiments, the compound has the structure of Formula I-G:

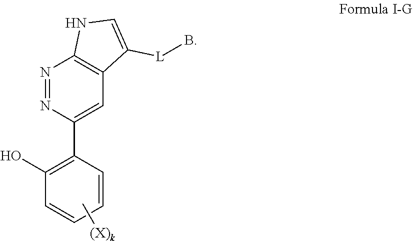

In some embodiments, the compound has the structure of Formula I-H:

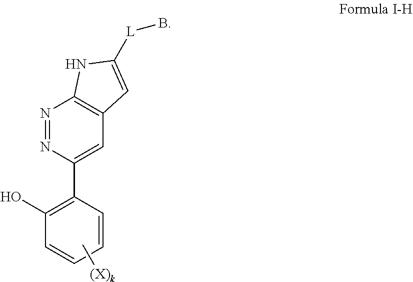

In some embodiments, the degradation moiety, B, has the structure of Formula A-1:

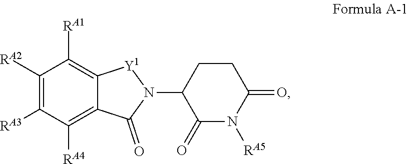

where
Y¹ is

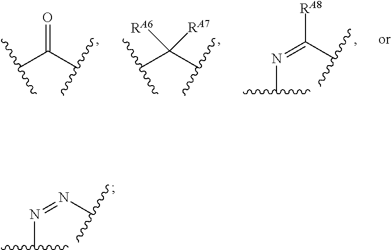

R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or
R^{A6} and R^{A7}, together with the carbon atom to which each is bound, combine to form optionally substituted C₃-C₆ carbocyclyl or optionally substituted C₂-C₅ heterocyclyl; or R^{A6} and R^{A7}, together with the carbon atom to which each is bound, combine to form optionally substituted C₃-C₆ carbocyclyl or optionally substituted C₂-C₅ heterocyclyl;
R^{A8} is H, optionally substituted C₁-C₆ alkyl, or optionally substituted C₁-C₆ heteroalkyl;
each of R^{A1}, R^{A2}, R^{A3} and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form

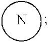

and

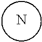

is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3} and R^{A4} is A², or

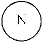

is substituted with A²; and
A² is a bond between the degradation moiety and the linker.

In some embodiments, R^{A5} is H or methyl. In some embodiments, R^{A5} is H.

In some embodiments, each of R^{A1}, R^{A2}, R^{A3}, and R^{A4} is, independently, H or A².

In some embodiments, R^{A1} is A² and each of R^{A2}, R^{A3}, and R^{A4} is H.

In some embodiments, R^{A2} is A² and each of R^{A1}, R^{A3}, and R^{A4} is H.

In some embodiments, R^{A3} is A² and each of R^{A1}, R^{A2}, and R^{A4} is H.

In some embodiments, R^{A4} is A² and each of R^{A1}, R^{A2}, and R^{A3} is H.

In some embodiments, Y¹ is or

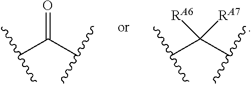

In some embodiments, R^{A6} is H. In some embodiments, R^{A7} is H.

In some embodiments, Y¹ is

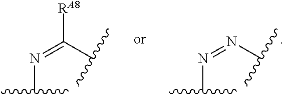

In some embodiments, R^{A8} is H or optionally substituted C₁-C₆ alkyl. In some embodiments, R^{A8} is H or methyl. In some embodiments, R^{A8} is methyl.

In some embodiments, the degradation moiety includes the structure of Formula A2:

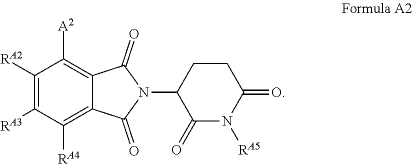

In some embodiments, where the degradation moiety is

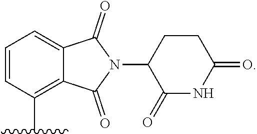 [SMILES: *c1cccc2c1C(=O)N(C1CCC(=O)NC1=O)C2=O]

In some embodiments, the degradation moiety includes the structure of Formula A4:

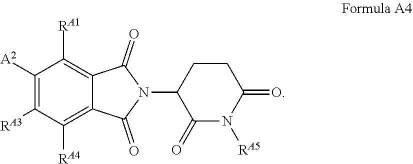

In some embodiments, the degradation moiety is

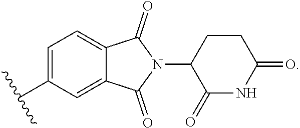 [SMILES: *c1ccc2c(c1)C(=O)N(C1CCC(=O)NC1=O)C2=O]

In some embodiments, the degradation moiety includes the structure of Formula A5:

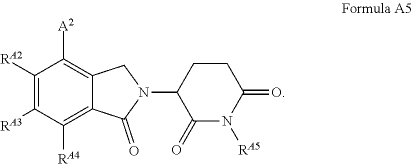

In some embodiments, the degradation moiety includes the structure of Formula A6:

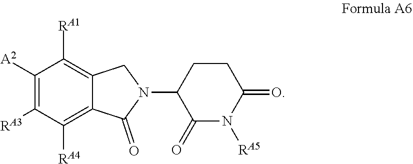

In some embodiments, the degradation moiety includes the structure of Formula A8:

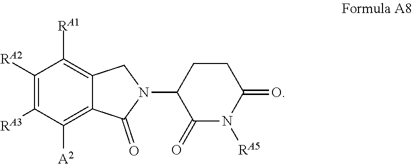

In some embodiments, the degradation moiety includes the structure of Formula A10:

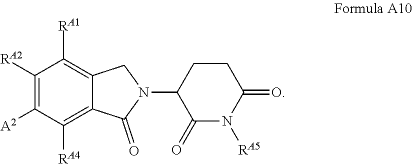

In some embodiments, the degradation moiety includes the structure of

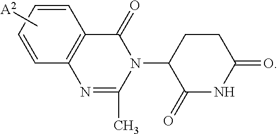

In some embodiments, the degradation moiety includes the structure of

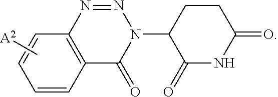

In some embodiments, the degradation moiety has the structure of Formula C:

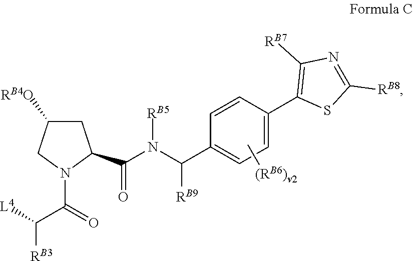

where
L⁴ is —N(R^{B1})(R^{B2}),

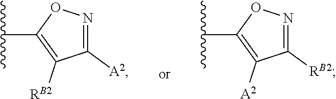

R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₅-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₅-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₆ heterocyclyl, optionally substituted C₅-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
where one and only one of R^{B1}, R^{B3}, and R^{B6} is A²,
or a pharmaceutically acceptable salt thereof.

In some embodiments, the degradation moiety has the structure of Formula C1:

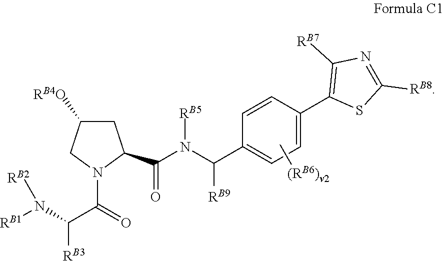

In some embodiments, the degradation moiety is

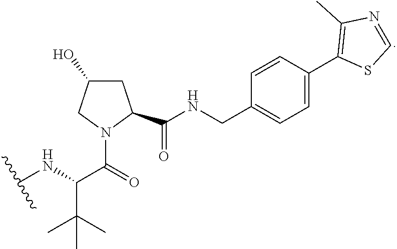 [SMILES: *N[C@H](C(=O)N1C[C@H](O)C[C@H]1C(=O)NCc1ccc(-c2scnc2C)cc1)C(C)(C)C]

In some embodiments, the degradation moiety is

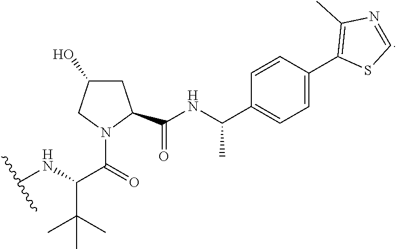 [SMILES: *N[C@H](C(=O)N1C[C@H](O)C[C@H]1C(=O)N[C@@H](C)c1ccc(-c2scnc2C)cc1)C(C)(C)C]

In some embodiments, the degradation moiety is

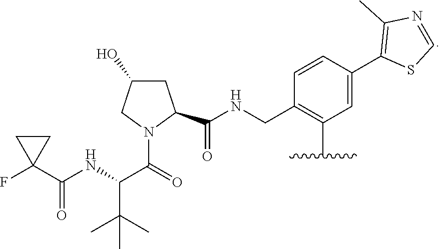 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, the degradation moiety has the structure of Formula C2:

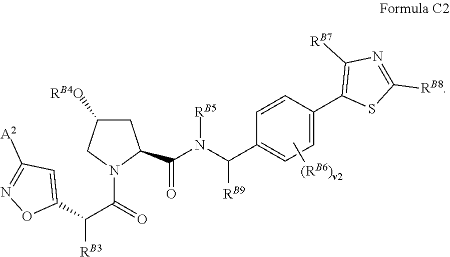

In some embodiments, R^{B9} is optionally substituted C₁-C₆ alkyl. In some embodiments, R^{B9} is methyl.

In some embodiments, R^{B9} is bonded to (S)-stereogenic center.

In some embodiments, the degradation moiety is

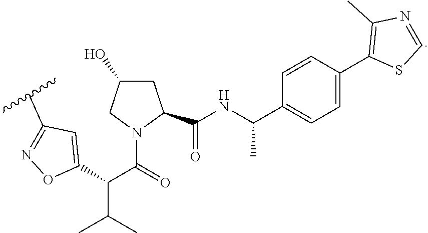 [SMILES: *c1cc([C@H](C(=O)N2C[C@H](O)C[C@H]2C(=O)N[C@@H](C)c2ccc(-c3scnc3C)cc2)C(C)C)on1]

In some embodiments, the linker has the structure of Formula II:

A¹-(B¹)_{f}—(C¹)_{g}—(B²)ₕ-(D)-(B³)ᵢ—(C²)ⱼ—(B⁴)ₖ-A², Formula II
or a pharmaceutically acceptable salt thereof,
where
A¹ is a bond between the linker and ring system A;
A² is a bond between the degradation moiety and the linker;
each of B¹, B², B³, and B⁴ is, independently, optionally substituted C₁-C₄ alkyl, optionally substituted C₅-C₁₀ aryl, optionally substituted C₅-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, O, S, S(O)₂, or NR^{N}; and D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C²)ⱼ—(B⁴)ₖ-A².

In some embodiments, each of B¹, B², B³, and B⁴ is, independently, optionally substituted C₁-C₂ alkyl, optionally substituted C₁-C₃ heteroalkyl, optionally substituted C₂-C₆ heterocyclyl, or NR^{N}.

In some embodiments, each R^{N} is, independently, H or optionally substituted C₁-C₄ alkyl.

In some embodiments, each R^{N} is, independently, H or CH₃.

In some embodiments, each of B¹ and B⁴ is, independently,

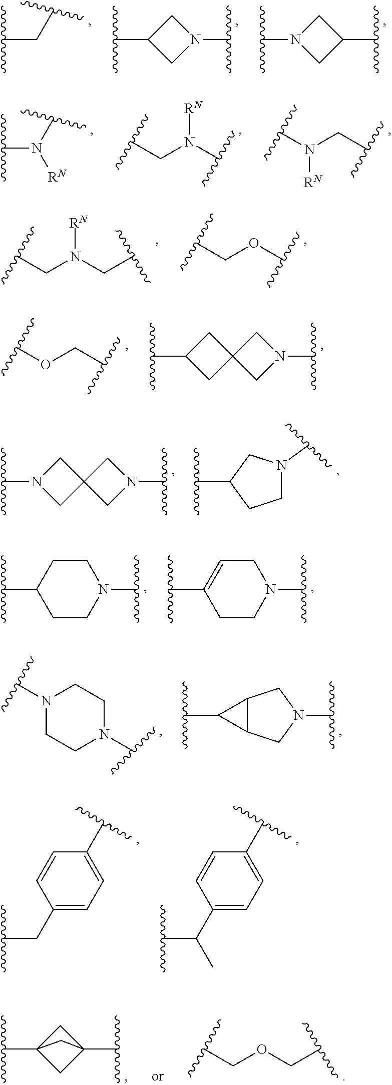

In some embodiments, each of B¹ and B⁴ is, independently,

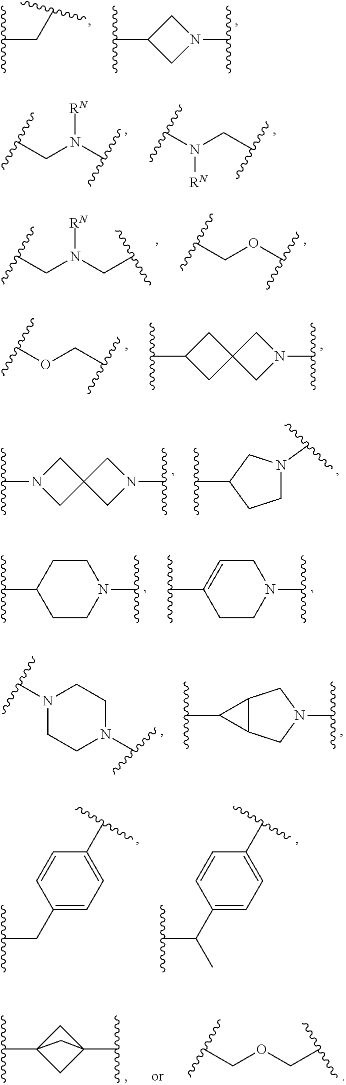

In some embodiments, B¹ is

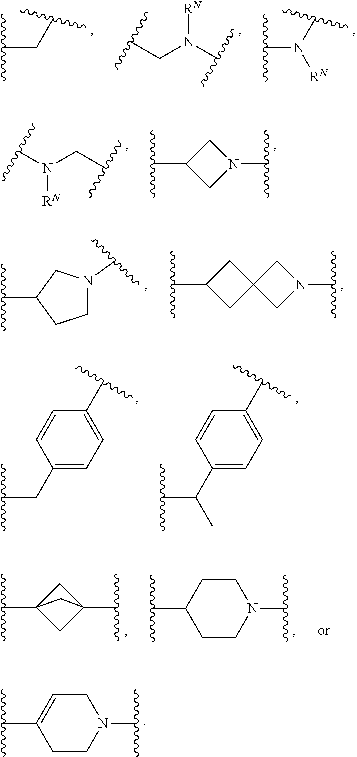

In some embodiments, B¹ is

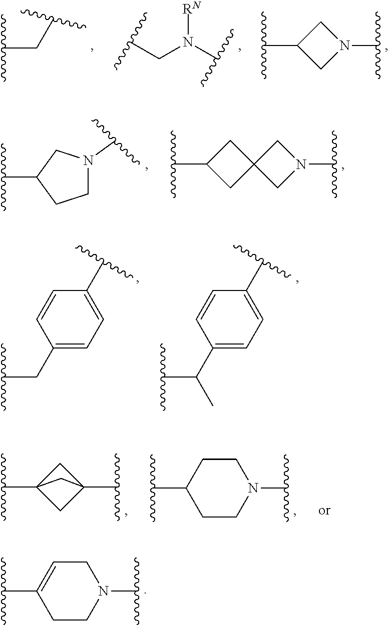

In some embodiments, B⁴ is

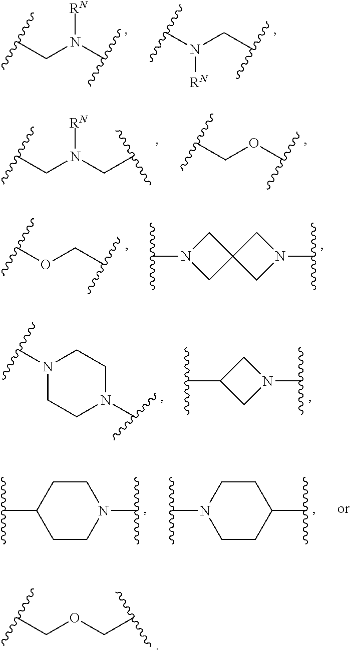

In some embodiments, B⁴ is

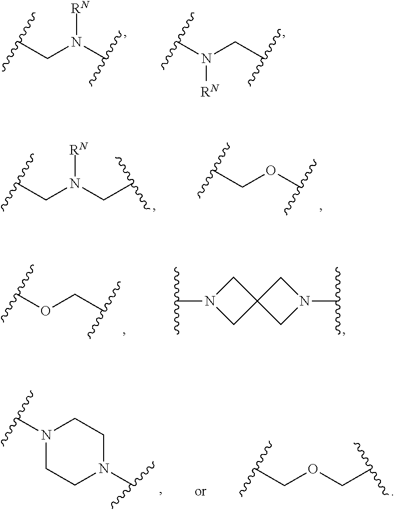

In some embodiments, each of C¹ and C² is

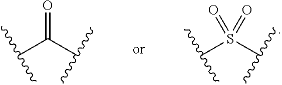

In some embodiments, C¹ is

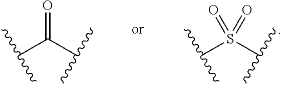

In some embodiments, C² is

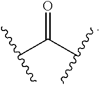 [SMILES: *C(*)=O]

In some embodiments, B² is optionally substituted C₁-C₄ alkyl. In some embodiments, B² is optionally substituted C₂-C₆ heterocyclyl.

In some embodiments, B² is

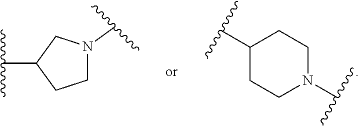 [SMILES: *C1CCN(*)C1 ; *C1CCN(*)CC1]

In some embodiments, D is optionally substituted C₁-C₁₀ alkyl.

In some embodiments, f is 1. In some embodiments, g is 0. In some embodiments, g is 1. In some embodiments, h is 0. In some embodiments, h is 1. In some embodiments, i is 0. In some embodiments, i is 1. In some embodiments, j is 0. In some embodiments, j is 1. In some embodiments, k is 0. In some embodiments, k is 1.

In some embodiments, the linker has the structure of

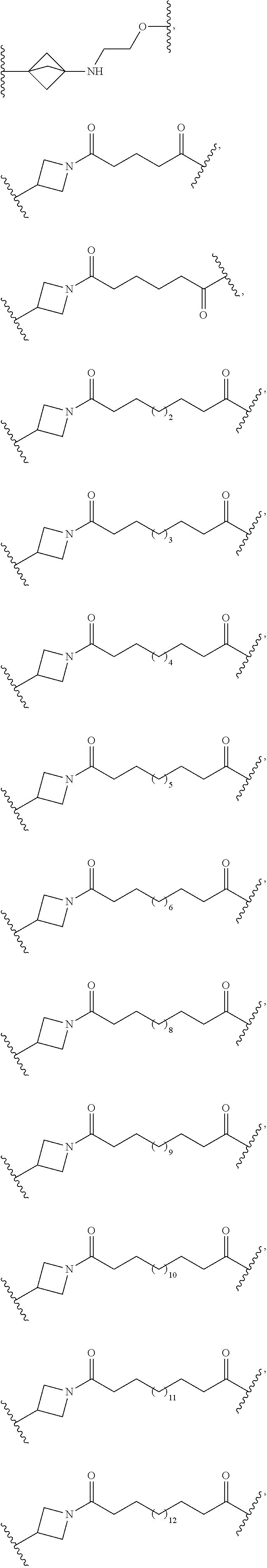

[chemical structure]

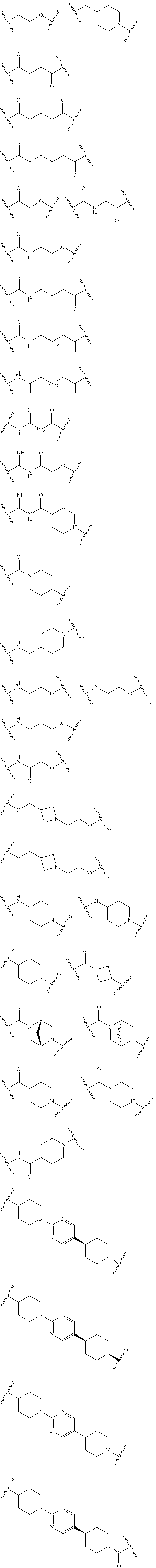

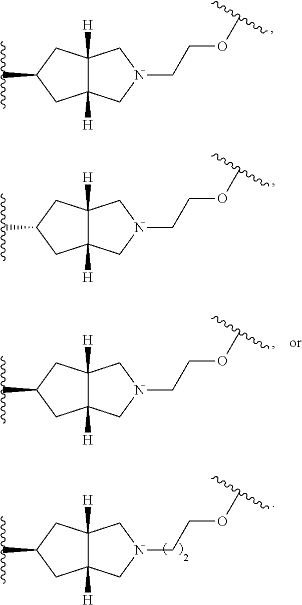

In some embodiments, the linker has the structure of

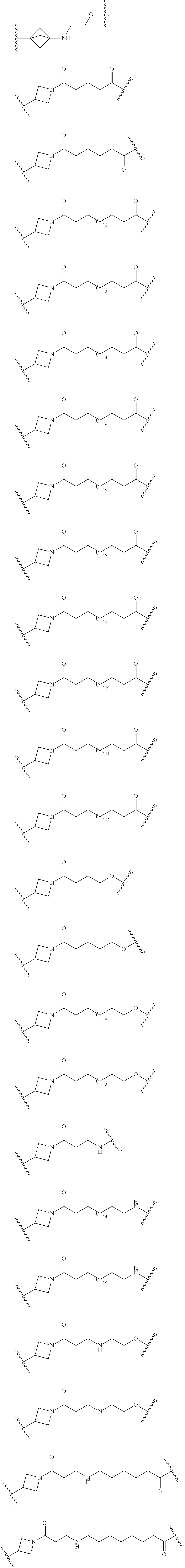

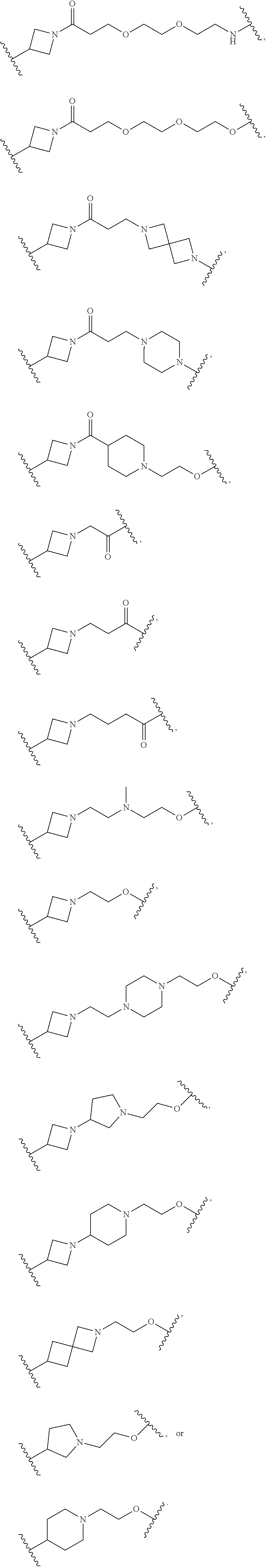

In some embodiments, the shortest chain of atoms connecting two valencies of the linker is 2 to 10 atoms long.

In some embodiments, the shortest chain of atoms connecting two valencies of the linker is 6 atoms long.

In some embodiments, the linker has the structure of

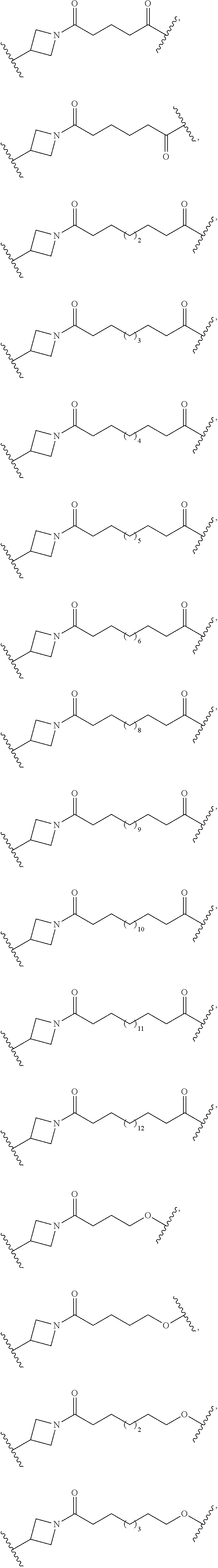

[chemical structure]

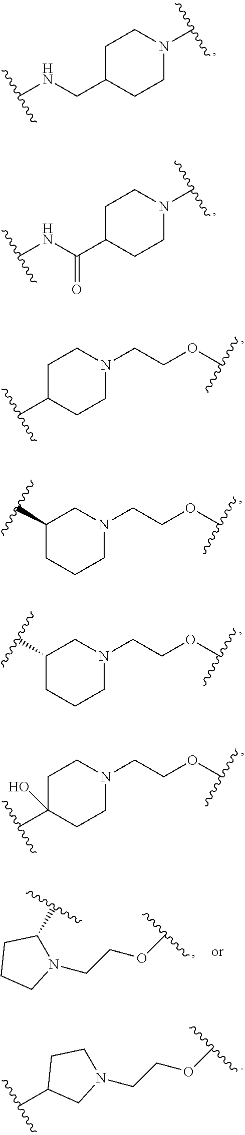

In some embodiments, the linker has the structure of

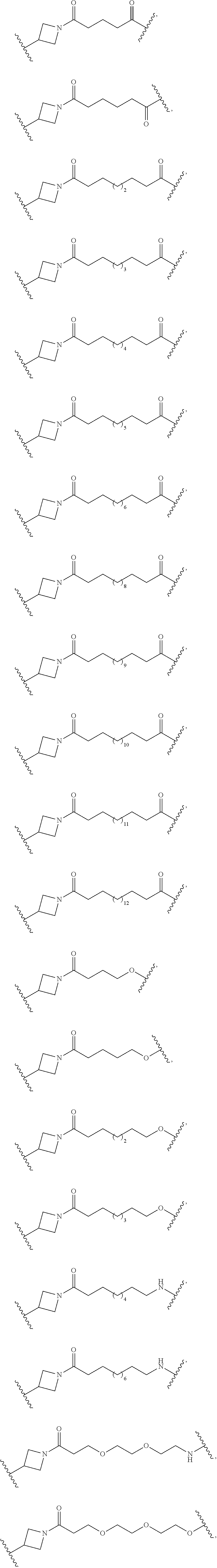

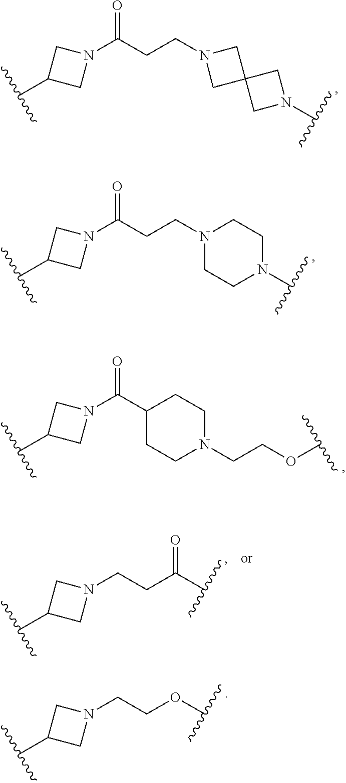 [SMILES: *C1CN(C(=O)CCN2CC3(CN(*)C3)C2)C1 ; *C1CN(C(=O)CCN2CCN(*)CC2)C1 ; *OCCN1CCC(C(=O)N2CC(*)C2)CC1 ; *C(=O)CCN1CC(*)C1 ; *OCCN1CC(*)C1]

In some embodiments, the linker has a structure of the linker in any one of compounds 1-310 in Table 1 (e.g., of any of the compounds with a ratio of BRG1 IC₅₀ to BRM IC₅₀ of at least 5 (e.g., at least 7, 10, 15, 20, 25, or 30)). In some embodiments, the linker has a structure of the linker in any one of compounds 1-310 in Table 1 (e.g., of any of the compounds with a BRM IC₅₀ of ++ or better (e.g., +++ or ++++(e.g., ++++))). In some embodiments, the linker has a structure of the linker in any one of compounds 1-310 in Table 1 (e.g., of any of the compounds with a BRM IC₅₀ of ++ or better (e.g., +++ or ++++(e.g., ++++)) and with a ratio of BRG1 IC₅₀ to BRM IC₅₀ of at least 5 (e.g., at least 7, 10, 15, 20, 25, or 30)).

In an aspect, the invention features a compound selected from the group consisting of 1-310 in Table 1 and pharmaceutically acceptable salts thereof. In some embodiments, the compound is any one of compounds 1-310 in Table 1 with a ratio of BRG1 IC₅₀ to BRM IC₅₀ of at least 5 (e.g., at least 7, 10, 15, 20, 25, or 30) or a pharmaceutically acceptable salt thereof. In some embodiments, the compound is any one of compounds 1-310 in Table 1 with a BRM IC₅₀ of ++ or better as found in Table 19 (e.g., +++ or ++++(e.g., ++++)) or a pharmaceutically acceptable salt thereof. In some embodiments, the compound is any one of compounds 1-310 in Table 1 a BRM IC₅₀ of ++ or better as found in Table 19 (e.g., +++ or ++++(e.g., ++++)) and with a ratio of BRG1 IC₅₀ to BRM IC₅₀ of at least 5 (e.g., at least 7, 10, 15, 20, 25, or 30) or a pharmaceutically acceptable salt thereof.

TABLE 1
Compounds of the Invention

| # | Compound |
|---|---|
| 1 | 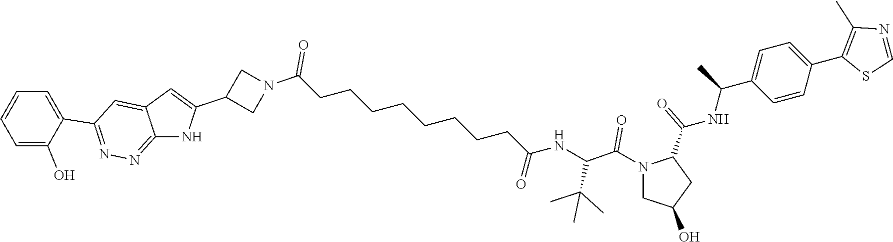 |
| 2 | 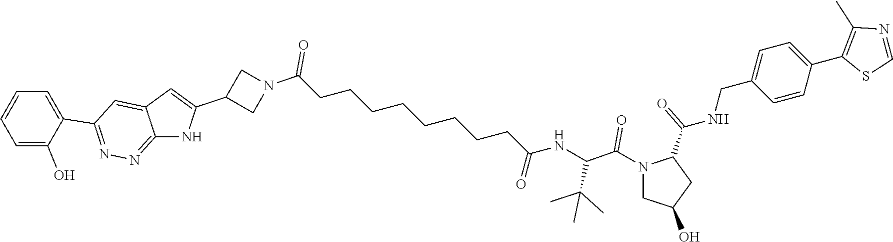 |
| 3 | 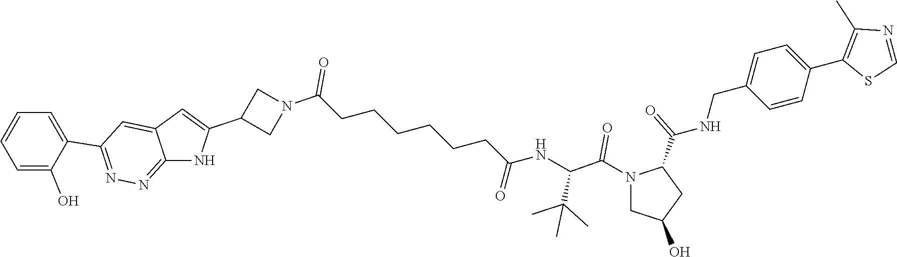 [SMILES: Cc1ncsc1-c1ccc(CNC(=O)[C@@H]2C[C@@H](O)CN2C(=O)[C@@H](NC(=O)CCCCCCC(=O)N2CC(c3cc4cc(-c5ccccc5O)nnc4[nH]3)C2)C(C)(C)C)cc1] |
| 4 | 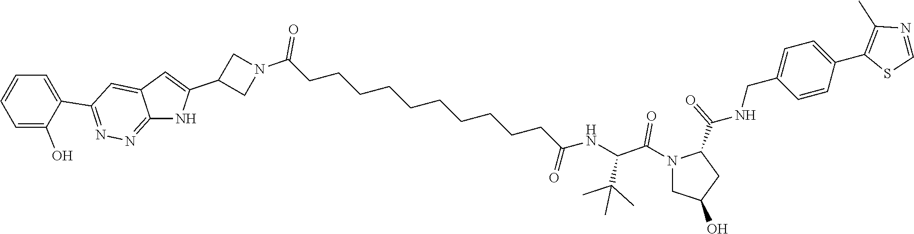 [SMILES: Cc1ncsc1-c1ccc(CNC(=O)[C@@H]2C[C@@H](O)CN2C(=O)[C@@H](NC(=O)CCCCCCCCCCC(=O)N2CC(c3cc4cc(-c5ccccc5O)nnc4[nH]3)C2)C(C)(C)C)cc1] |
| 5 | 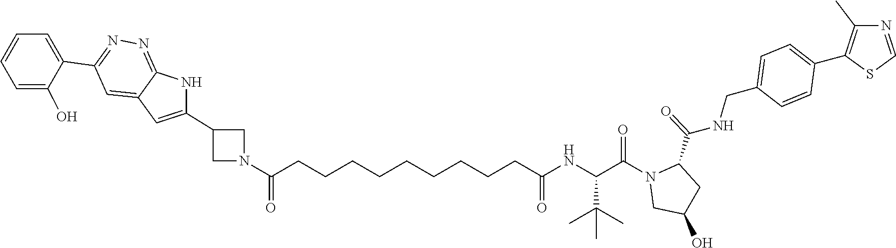 [SMILES: Cc1ncsc1-c1ccc(CNC(=O)[C@@H]2C[C@@H](O)CN2C(=O)[C@@H](NC(=O)CCCCCCCCCC(=O)N2CC(c3cc4cc(-c5ccccc5O)nnc4[nH]3)C2)C(C)(C)C)cc1] |
| 6 | 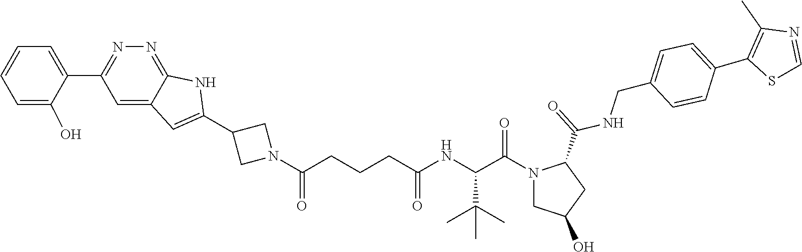 [SMILES: Cc1ncsc1-c1ccc(CNC(=O)[C@@H]2C[C@@H](O)CN2C(=O)[C@@H](NC(=O)CCCC(=O)N2CC(c3cc4cc(-c5ccccc5O)nnc4[nH]3)C2)C(C)(C)C)cc1] |
| 7 | 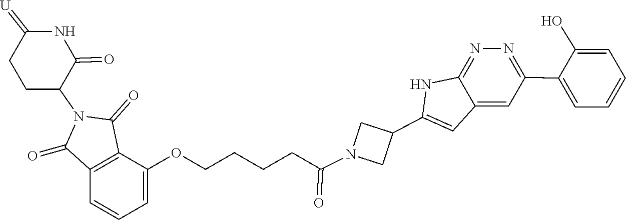 |
| 8 | 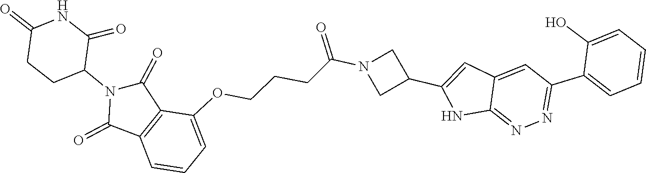 [SMILES: O=C1CCC(N2C(=O)c3cccc(OCCCC(=O)N4CC(c5cc6cc(-c7ccccc7O)nnc6[nH]5)C4)c3C2=O)C(=O)N1] |
| 9 | 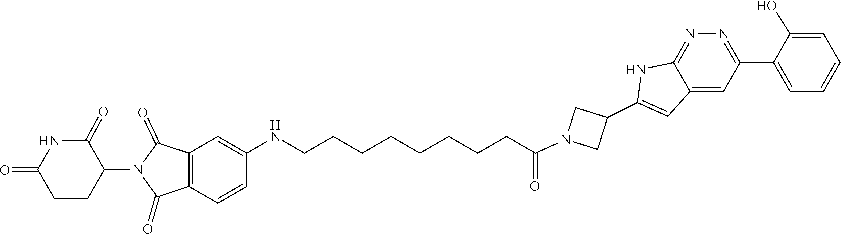 [SMILES: O=C1CCC(N2C(=O)c3ccc(NCCCCCCCCC(=O)N4CC(c5cc6cc(-c7ccccc7O)nnc6[nH]5)C4)cc3C2=O)C(=O)N1] |
| 10 | 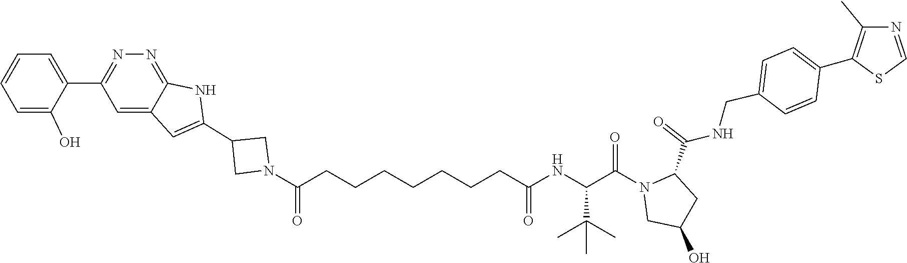 [SMILES: Cc1ncsc1-c1ccc(CNC(=O)[C@@H]2C[C@@H](O)CN2C(=O)[C@@H](NC(=O)CCCCCCCC(=O)N2CC(c3cc4cc(-c5ccccc5O)nnc4[nH]3)C2)C(C)(C)C)cc1] |
| 11 | 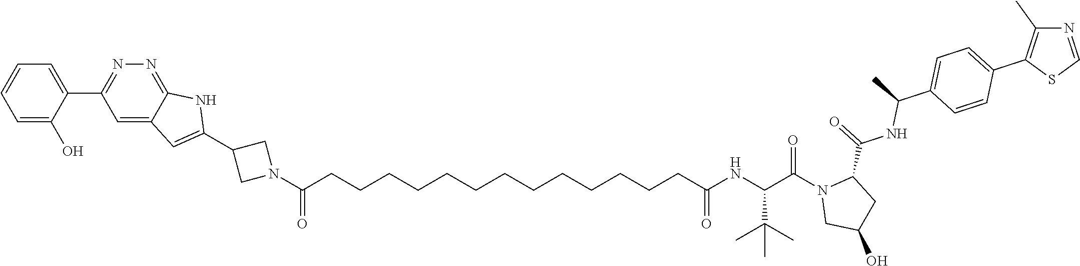 [SMILES: Cc1ncsc1-c1ccc([C@H](C)NC(=O)[C@@H]2C[C@@H](O)CN2C(=O)[C@@H](NC(=O)CCCCCCCCCCCCCC(=O)N2CC(c3cc4cc(-c5ccccc5O)nnc4[nH]3)C2)C(C)(C)C)cc1] |
| 12 | 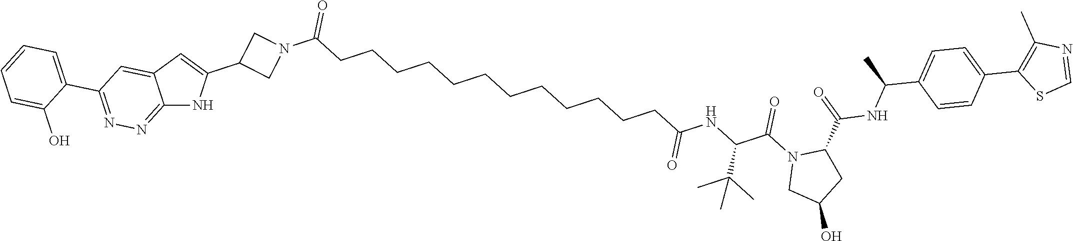 [SMILES: Cc1ncsc1-c1ccc([C@H](C)NC(=O)[C@@H]2C[C@@H](O)CN2C(=O)[C@@H](NC(=O)CCCCCCCCCCCCC(=O)N2CC(c3cc4cc(-c5ccccc5O)nnc4[nH]3)C2)C(C)(C)C)cc1] |
| 13 | 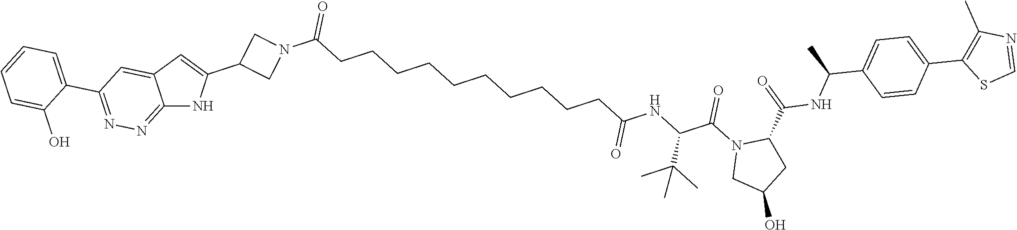 [SMILES: Cc1ncsc1-c1ccc([C@H](C)NC(=O)[C@@H]2C[C@@H](O)CN2C(=O)[C@@H](NC(=O)CCCCCCCCCCC(=O)N2CC(c3cc4cc(-c5ccccc5O)nnc4[nH]3)C2)C(C)(C)C)cc1] |
| 14 | 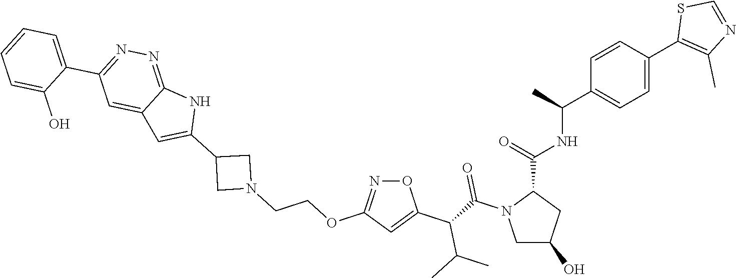 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5[nH]4)C3)no2)C(C)C)cc1] |
| 15 | 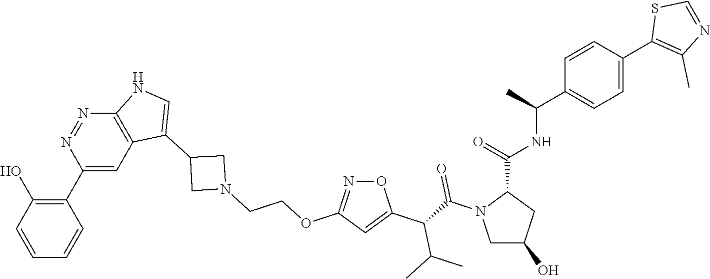 [SMILES: Cc1ncsc1-c1ccc([C@H](C)NC(=O)[C@@H]2C[C@@H](O)CN2C(=O)[C@@H](c2cc(OCCN3CC(c4c[nH]c5nnc(-c6ccccc6O)cc45)C3)no2)C(C)C)cc1] |
| 16 | 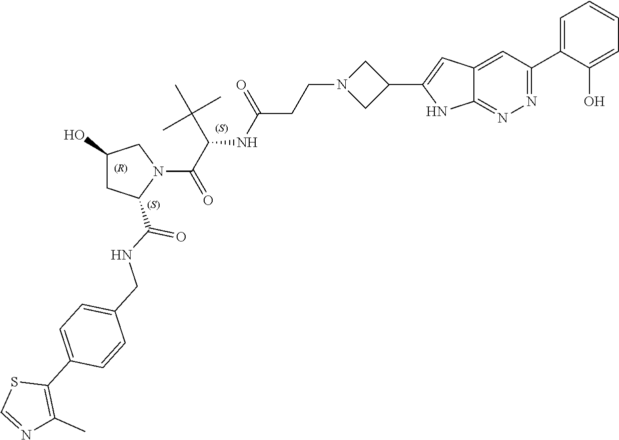 |
| 17 | 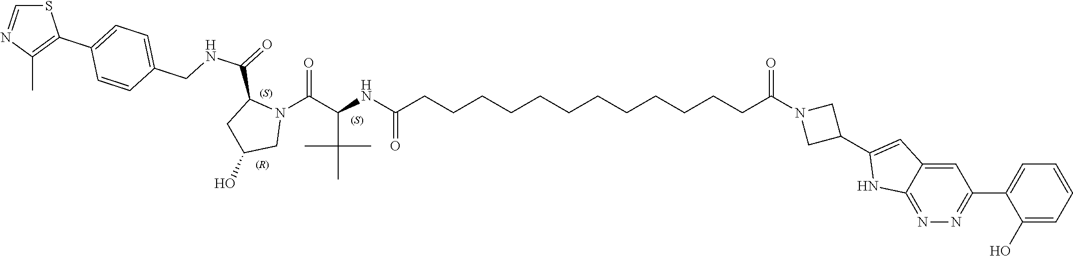 |
| 18 | 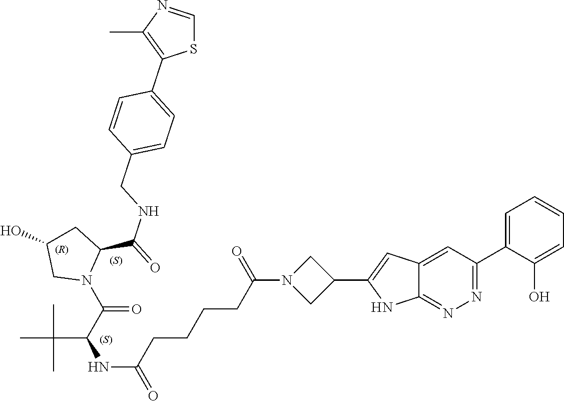 |
| 19 | 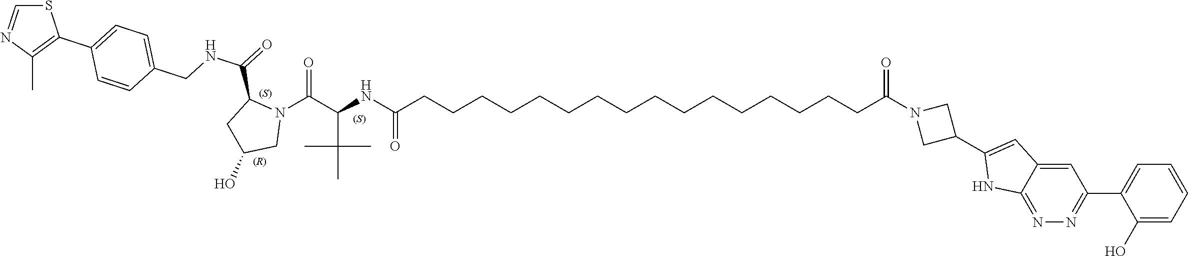 |
| 20 | 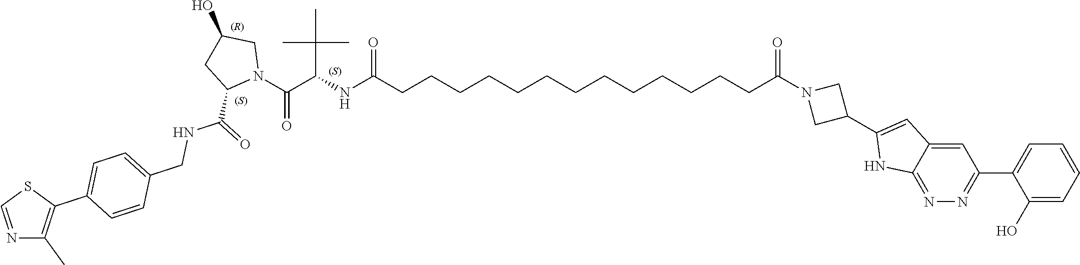 |
| 21 | 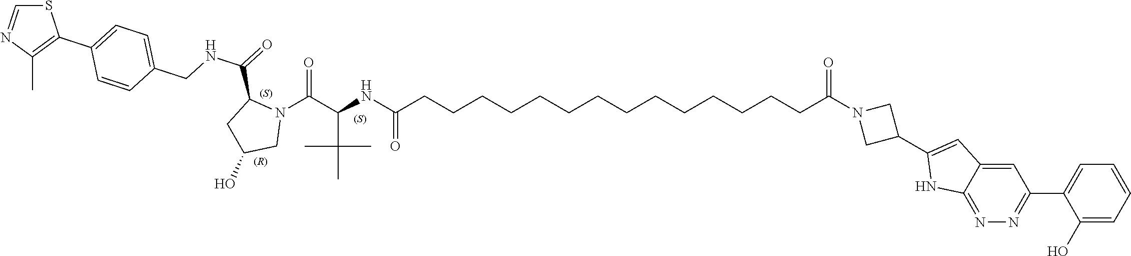 |
| 22 | 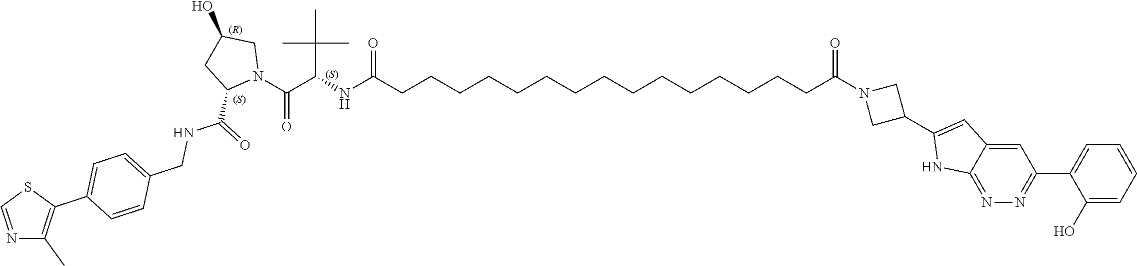 |
| 23 | 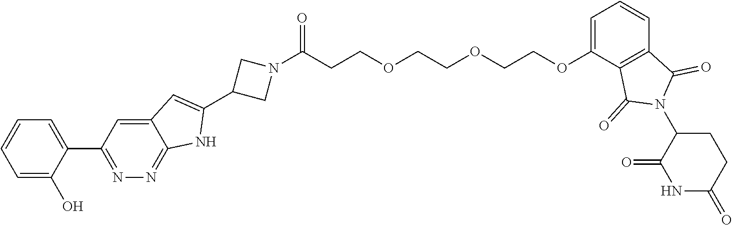 [SMILES: O=C1CCC(N2C(=O)c3cccc(OCCOCCOCCC(=O)N4CC(c5cc6cc(-c7ccccc7O)nnc6[nH]5)C4)c3C2=O)C(=O)N1] |
| 24 | 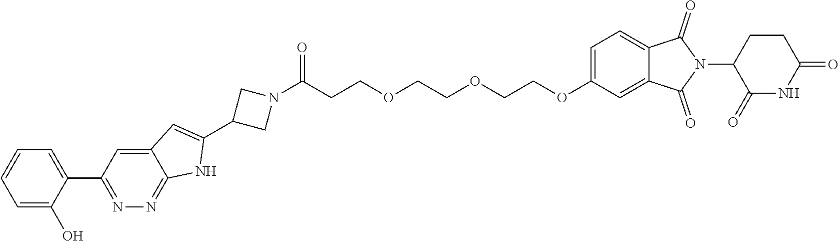 [SMILES: O=C1CCC(N2C(=O)c3ccc(OCCOCCOCCC(=O)N4CC(c5cc6cc(-c7ccccc7O)nnc6[nH]5)C4)cc3C2=O)C(=O)N1] |
| 25 | 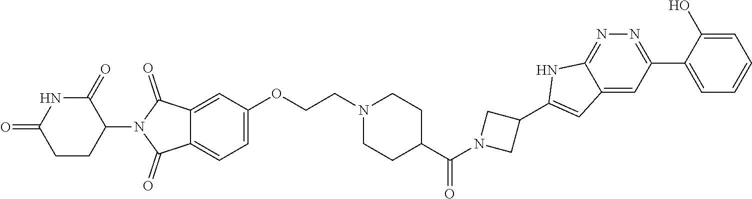 [SMILES: O=C1CCC(N2C(=O)c3ccc(OCCN4CCC(C(=O)N5CC(c6cc7cc(-c8ccccc8O)nnc7[nH]6)C5)CC4)cc3C2=O)C(=O)N1] |
| 26 | 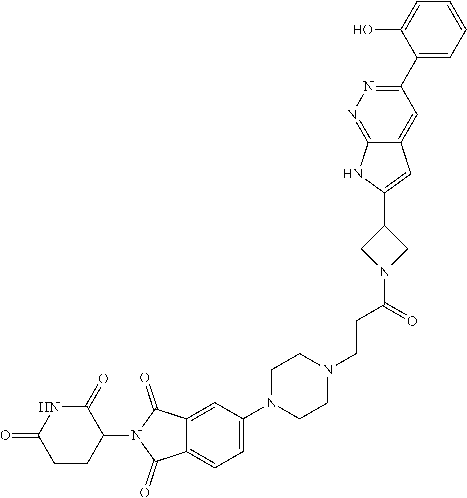 [SMILES: O=C1CCC(N2C(=O)c3ccc(N4CCN(CCC(=O)N5CC(c6cc7cc(-c8ccccc8O)nnc7[nH]6)C5)CC4)cc3C2=O)C(=O)N1] |
| 27 | 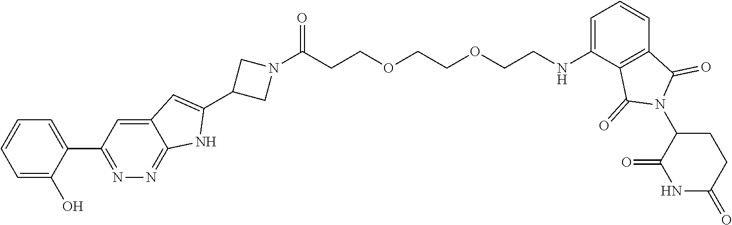 [SMILES: O=C1CCC(N2C(=O)c3cccc(NCCOCCOCCC(=O)N4CC(c5cc6cc(-c7ccccc7O)nnc6[nH]5)C4)c3C2=O)C(=O)N1] |
| 28 | 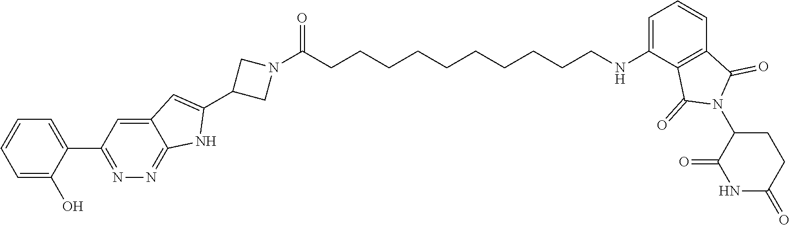 [SMILES: O=C1CCC(N2C(=O)c3cccc(NCCCCCCCCCCC(=O)N4CC(c5cc6cc(-c7ccccc7O)nnc6[nH]5)C4)c3C2=O)C(=O)N1] |
| 29 | 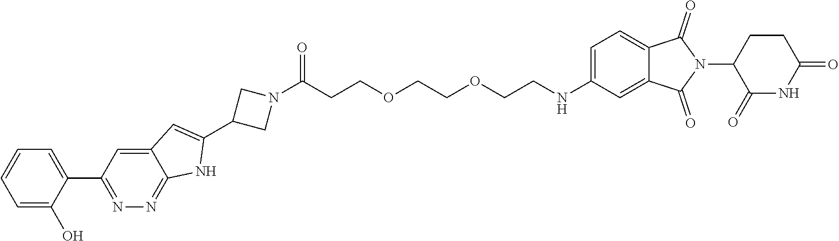 [SMILES: O=C1CCC(N2C(=O)c3ccc(NCCOCCOCCC(=O)N4CC(c5cc6cc(-c7ccccc7O)nnc6[nH]5)C4)cc3C2=O)C(=O)N1] |
| 30 | 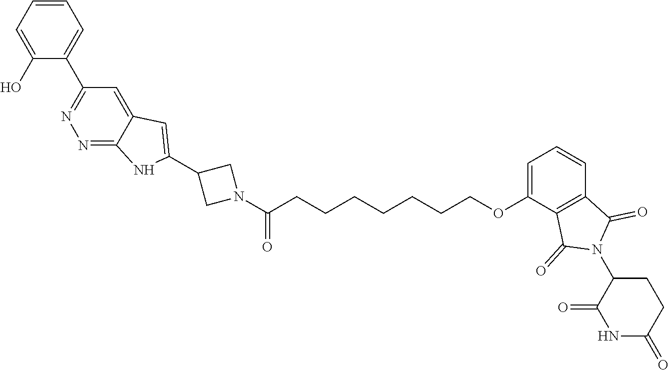 [SMILES: O=C1CCC(N2C(=O)c3cccc(OCCCCCCCC(=O)N4CC(c5cc6cc(-c7ccccc7O)nnc6[nH]5)C4)c3C2=O)C(=O)N1] |
| 31 | 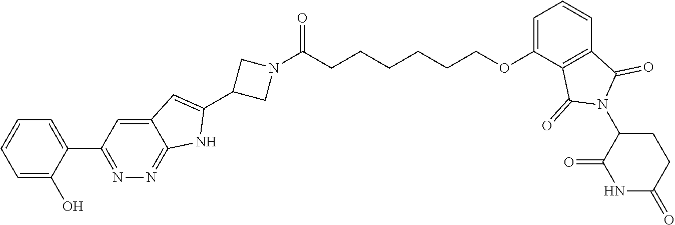 [SMILES: O=C1CCC(N2C(=O)c3cccc(OCCCCCCC(=O)N4CC(c5cc6cc(-c7ccccc7O)nnc6[nH]5)C4)c3C2=O)C(=O)N1] |
| 32 | 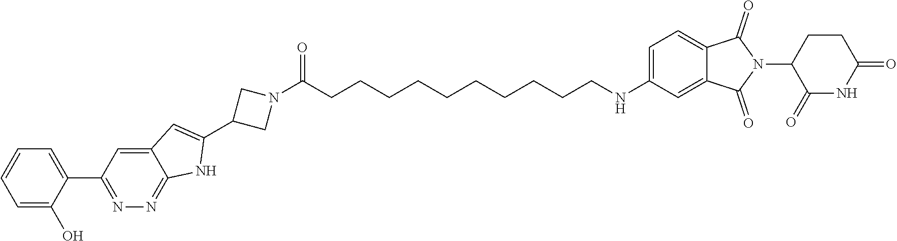 [SMILES: O=C1CCC(N2C(=O)c3ccc(NCCCCCCCCCCC(=O)N4CC(c5cc6cc(-c7ccccc7O)nnc6[nH]5)C4)cc3C2=O)C(=O)N1] |
| 33 | 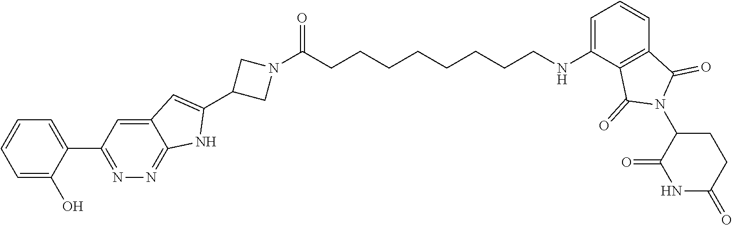 [SMILES: O=C1CCC(N2C(=O)c3cccc(NCCCCCCCCC(=O)N4CC(c5cc6cc(-c7ccccc7O)nnc6[nH]5)C4)c3C2=O)C(=O)N1] |
| 34 | 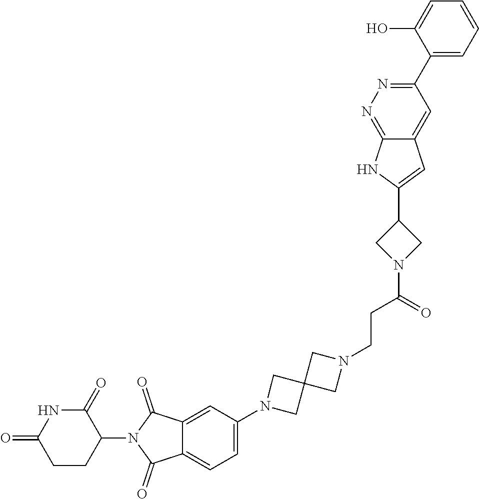 [SMILES: O=C1CCC(N2C(=O)c3ccc(N4CC5(CN(CCC(=O)N6CC(c7cc8cc(-c9ccccc9O)nnc8[nH]7)C6)C5)C4)cc3C2=O)C(=O)N1] |
| 35 | 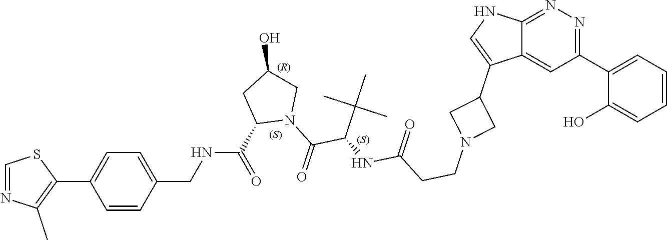 |
| 36 | 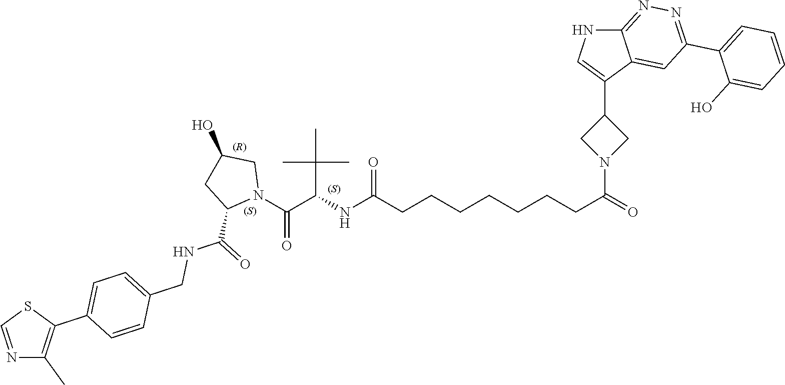 |
| 37 | 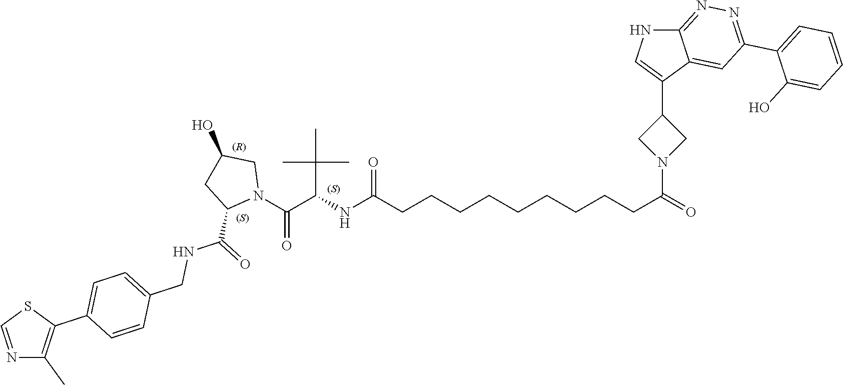 |
| 38 | 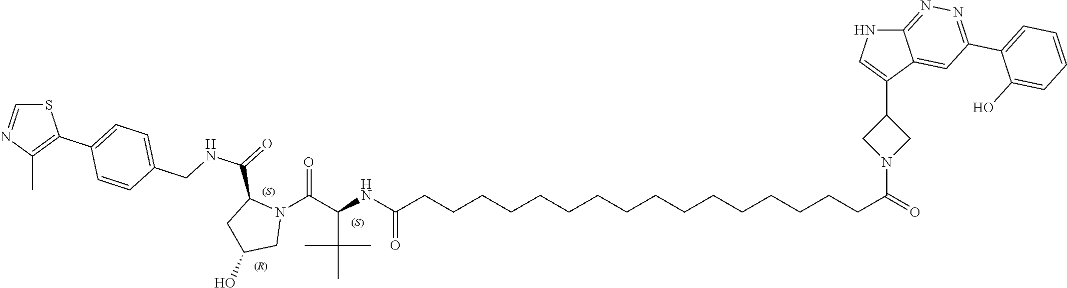 |
| 39 | 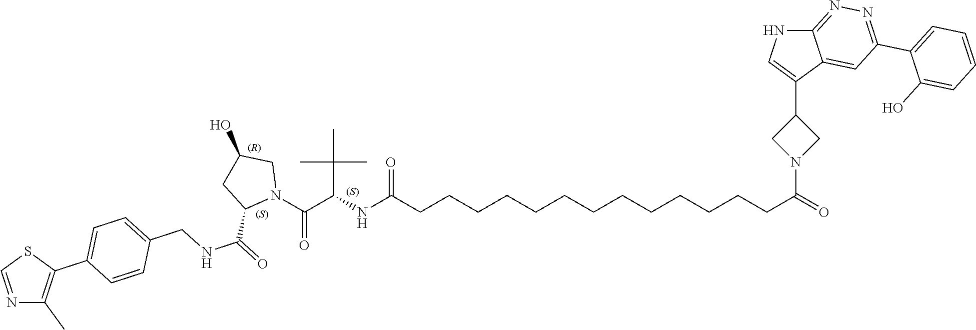 |
| 40 | 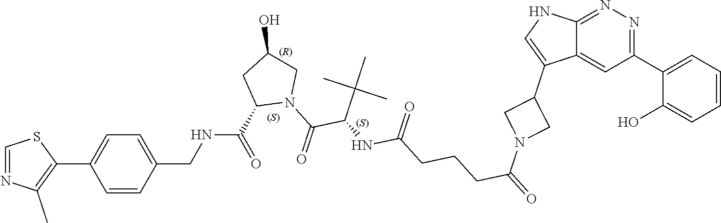 |
| 41 | 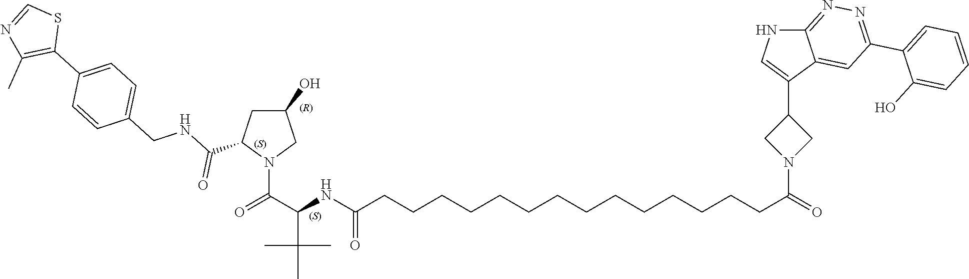 |
| 42 | 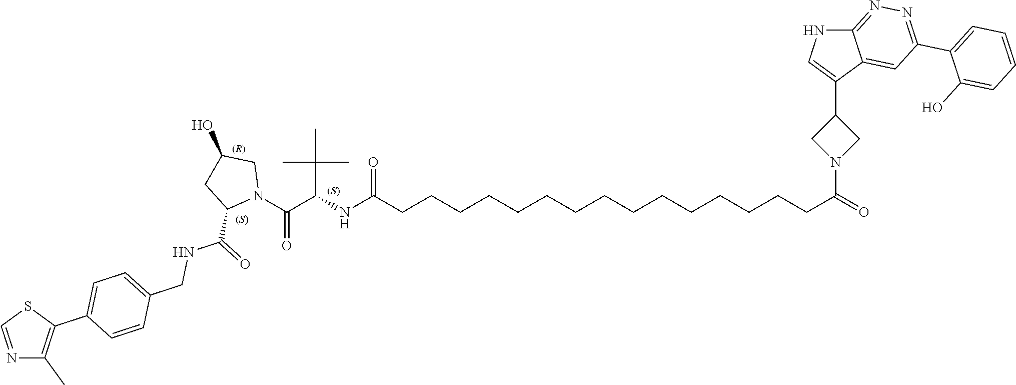 |
| 43 | 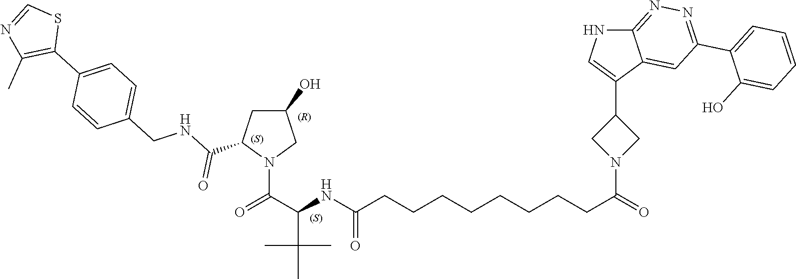 |
| 44 | 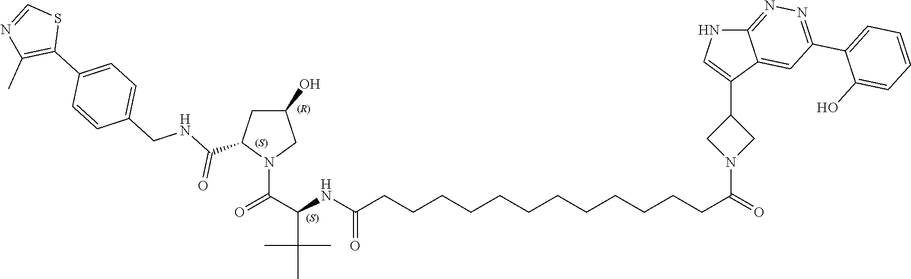 |
| 45 | 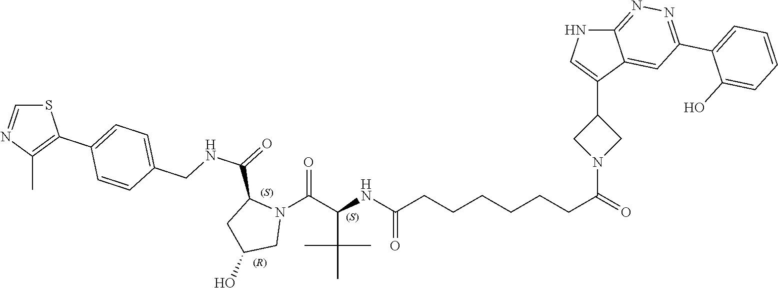 |
| 46 | 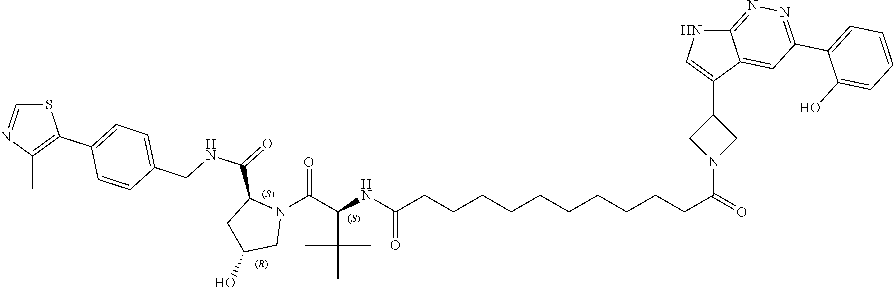 |
| 47 | 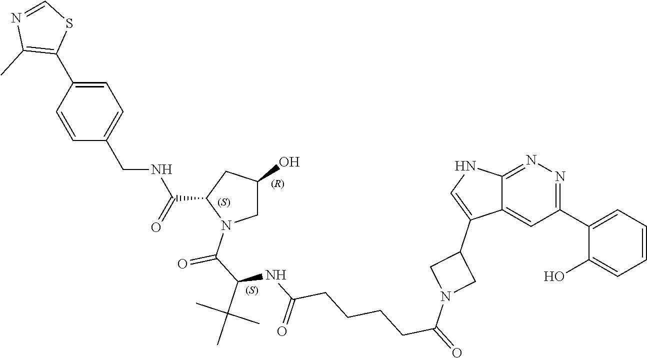 |
| 48 | 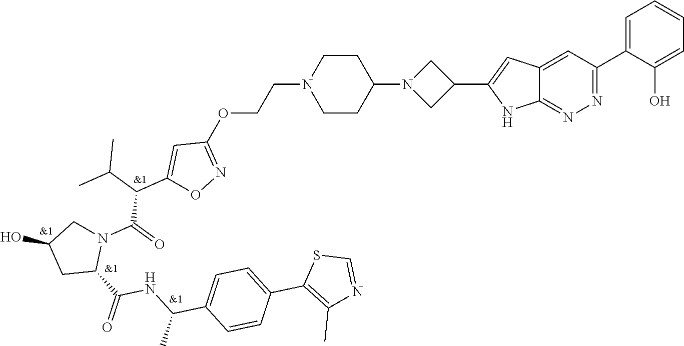 [SMILES: Cc1ncsc1-c1ccc([C@H](C)NC(=O)[C@@H]2C[C@@H](O)CN2C(=O)[C@@H](c2cc(OCCN3CCC(N4CC(c5cc6cc(-c7ccccc7O)nnc6[nH]5)C4)CC3)no2)C(C)C)cc1] |
| 49 | 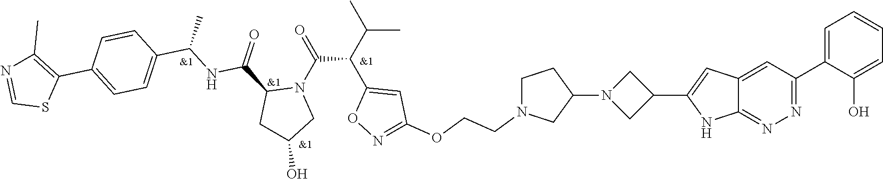 [SMILES: Cc1ncsc1-c1ccc([C@H](C)NC(=O)[C@@H]2C[C@@H](O)CN2C(=O)[C@@H](c2cc(OCCN3CCC(N4CC(c5cc6cc(-c7ccccc7O)nnc6[nH]5)C4)C3)no2)C(C)C)cc1] |
| 50 | 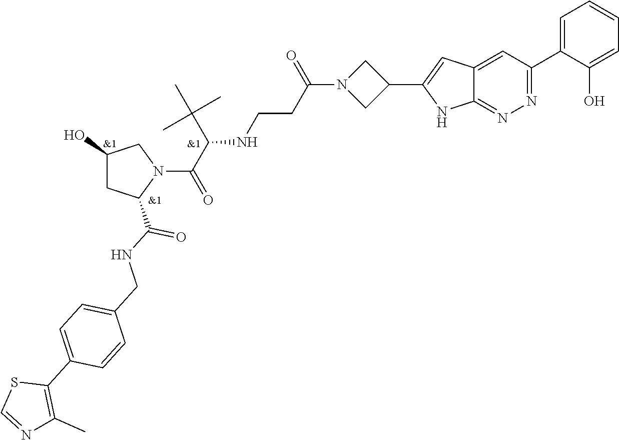 [SMILES: Cc1ncsc1-c1ccc(CNC(=O)[C@@H]2C[C@@H](O)CN2C(=O)[C@@H](NCCC(=O)N2CC(c3cc4cc(-c5ccccc5O)nnc4[nH]3)C2)C(C)(C)C)cc1] |
| 51 | 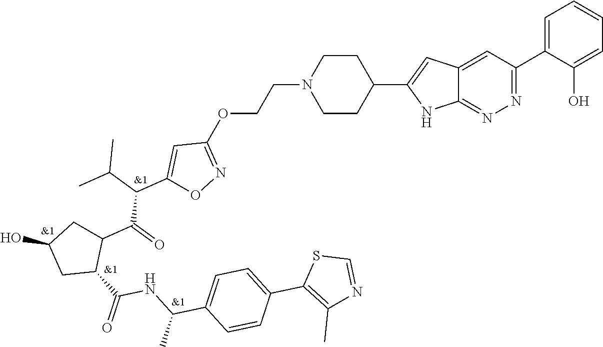 [SMILES: Cc1ncsc1-c1ccc([C@H](C)NC(=O)[C@@H]2C[C@@H](O)CC2C(=O)[C@@H](c2cc(OCCN3CCC(c4cc5cc(-c6ccccc6O)nnc5[nH]4)CC3)no2)C(C)C)cc1] |
| 52 | 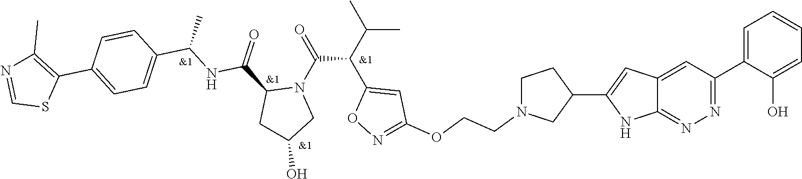 [SMILES: Cc1ncsc1-c1ccc([C@H](C)NC(=O)[C@@H]2C[C@@H](O)CN2C(=O)[C@@H](c2cc(OCCN3CCC(c4cc5cc(-c6ccccc6O)nnc5[nH]4)C3)no2)C(C)C)cc1] |
| 53 | 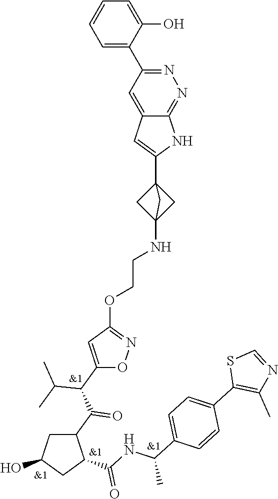 [SMILES: Cc1ncsc1-c1ccc([C@H](C)NC(=O)[C@@H]2C[C@@H](O)CC2C(=O)[C@@H](c2cc(OCCNC34CC(c5cc6cc(-c7ccccc7O)nnc6[nH]5)(C3)C4)no2)C(C)C)cc1] |
| 54 | 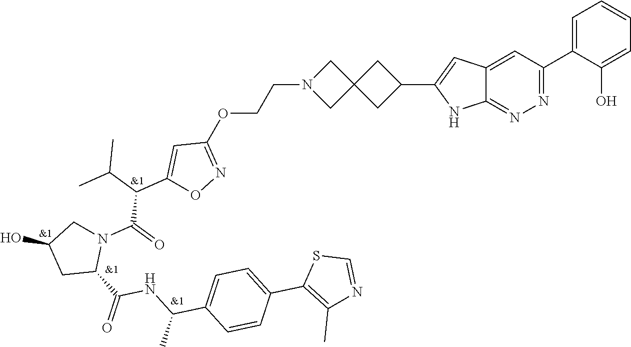 [SMILES: Cc1ncsc1-c1ccc([C@H](C)NC(=O)[C@@H]2C[C@@H](O)CN2C(=O)[C@@H](c2cc(OCCN3CC4(CC(c5cc6cc(-c7ccccc7O)nnc6[nH]5)C4)C3)no2)C(C)C)cc1] |
| 55 | 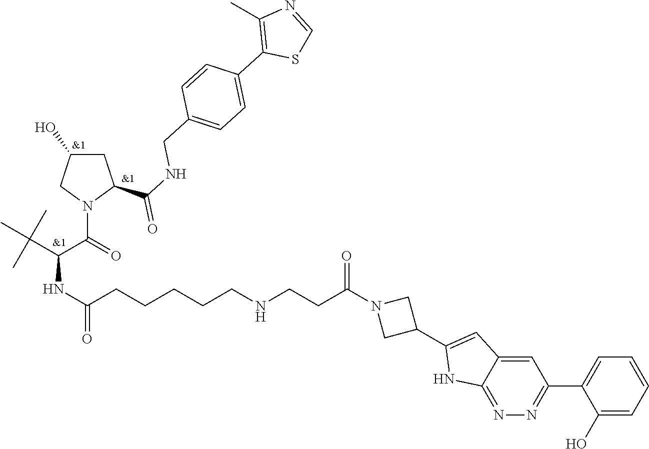 [SMILES: Cc1ncsc1-c1ccc(CNC(=O)[C@@H]2C[C@@H](O)CN2C(=O)[C@@H](NC(=O)CCCCCNCCC(=O)N2CC(c3cc4cc(-c5ccccc5O)nnc4[nH]3)C2)C(C)(C)C)cc1] |
| 56 | 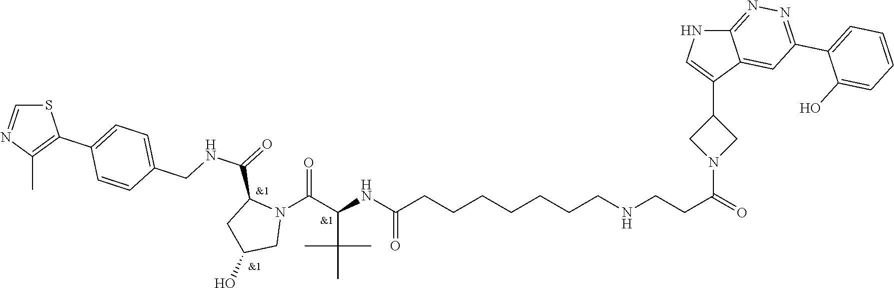 [SMILES: Cc1ncsc1-c1ccc(CNC(=O)[C@@H]2C[C@@H](O)CN2C(=O)[C@@H](NC(=O)CCCCCCCNCCC(=O)N2CC(c3c[nH]c4nnc(-c5ccccc5O)cc34)C2)C(C)(C)C)cc1] |
| 57 | 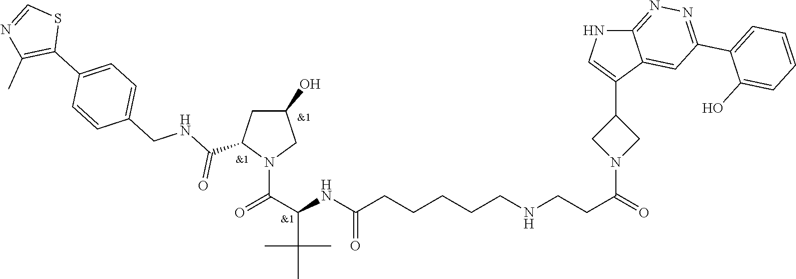 [SMILES: Cc1ncsc1-c1ccc(CNC(=O)[C@@H]2C[C@@H](O)CN2C(=O)[C@@H](NC(=O)CCCCCNCCC(=O)N2CC(c3c[nH]c4nnc(-c5ccccc5O)cc34)C2)C(C)(C)C)cc1] |
| 58 | 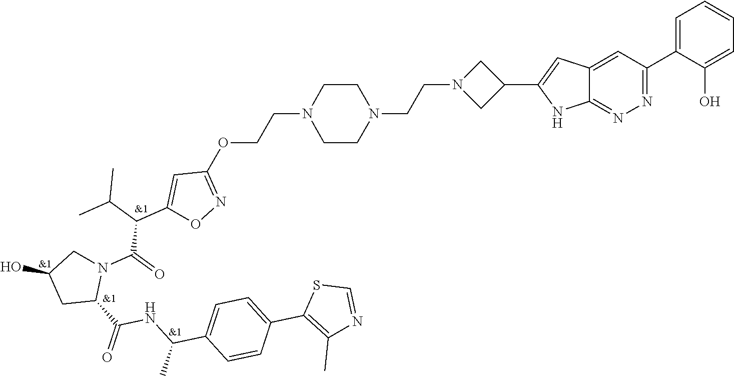 [SMILES: Cc1ncsc1-c1ccc([C@H](C)NC(=O)[C@@H]2C[C@@H](O)CN2C(=O)[C@@H](c2cc(OCCN3CCN(CCN4CC(c5cc6cc(-c7ccccc7O)nnc6[nH]5)C4)CC3)no2)C(C)C)cc1] |
| 59 | 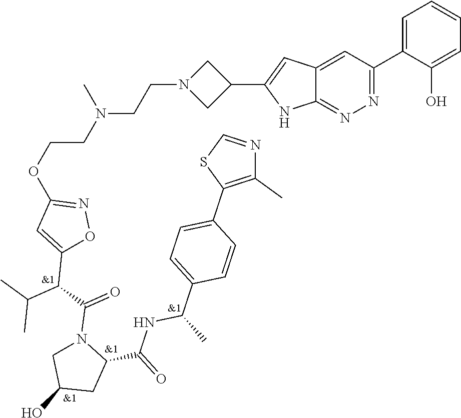 [SMILES: Cc1ncsc1-c1ccc([C@H](C)NC(=O)[C@@H]2C[C@@H](O)CN2C(=O)[C@@H](c2cc(OCCN(C)CCN3CC(c4cc5cc(-c6ccccc6O)nnc5[nH]4)C3)no2)C(C)C)cc1] |
| 60 | 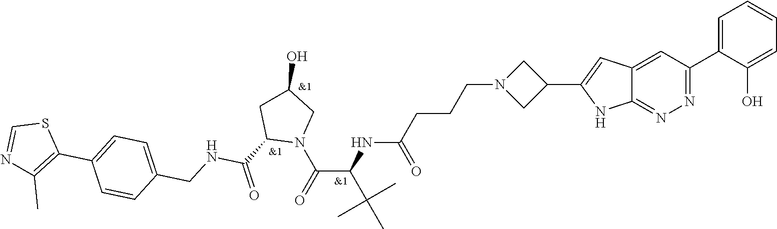 [SMILES: Cc1ncsc1-c1ccc(CNC(=O)[C@@H]2C[C@@H](O)CN2C(=O)[C@@H](NC(=O)CCCN2CC(c3cc4cc(-c5ccccc5O)nnc4[nH]3)C2)C(C)(C)C)cc1] |
| 61 | 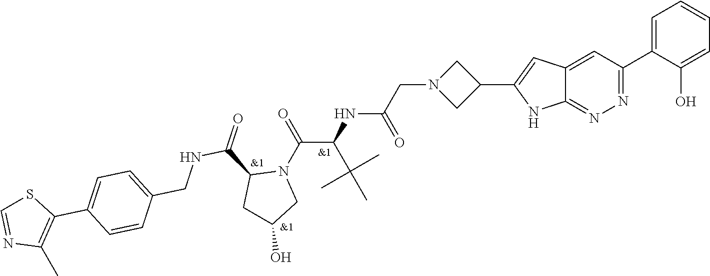 [SMILES: Cc1ncsc1-c1ccc(CNC(=O)[C@@H]2C[C@@H](O)CN2C(=O)[C@@H](NC(=O)CN2CC(c3cc4cc(-c5ccccc5O)nnc4[nH]3)C2)C(C)(C)C)cc1] |
| 62 | 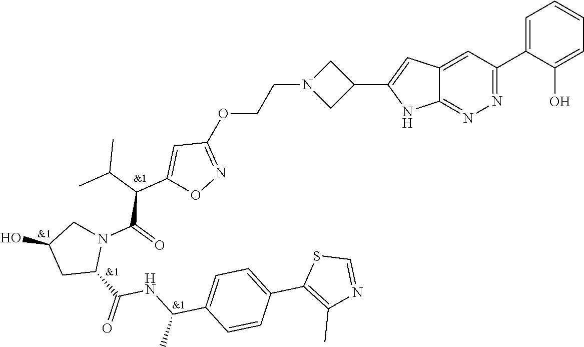 [SMILES: Cc1ncsc1-c1ccc([C@H](C)NC(=O)[C@@H]2C[C@@H](O)CN2C(=O)[C@H](c2cc(OCCN3CC(c4cc5cc(-c6ccccc6O)nnc5[nH]4)C3)no2)C(C)C)cc1] |
| 63 | 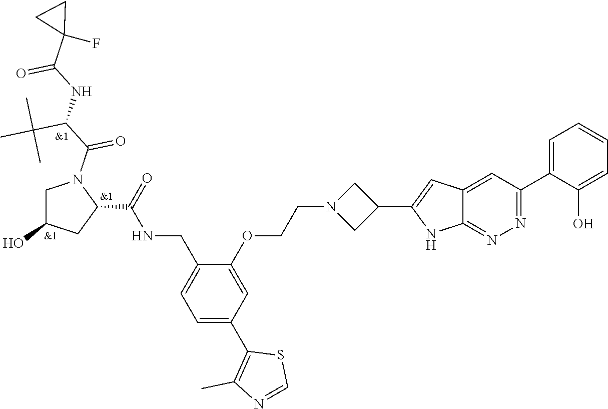 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CC(c3cc4cc(-c5ccccc5O)nnc4[nH]3)C2)c1] |
| 64 | 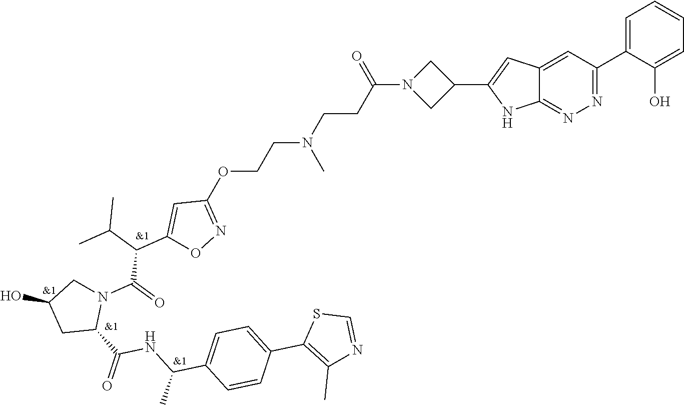 [SMILES: Cc1ncsc1-c1ccc([C@H](C)NC(=O)[C@@H]2C[C@@H](O)CN2C(=O)[C@@H](c2cc(OCCN(C)CCC(=O)N3CC(c4cc5cc(-c6ccccc6O)nnc5[nH]4)C3)no2)C(C)C)cc1] |
| 65 | 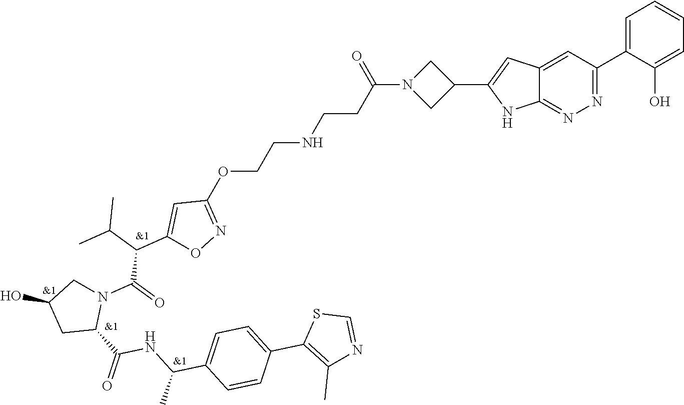 [SMILES: Cc1ncsc1-c1ccc([C@H](C)NC(=O)[C@@H]2C[C@@H](O)CN2C(=O)[C@@H](c2cc(OCCNCCC(=O)N3CC(c4cc5cc(-c6ccccc6O)nnc5[nH]4)C3)no2)C(C)C)cc1] |
| 66 | 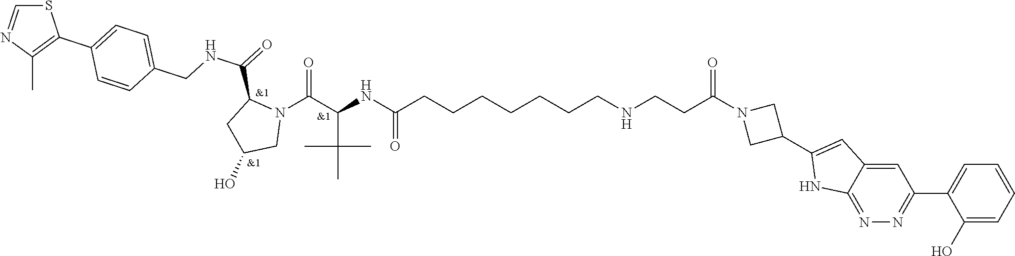 [SMILES: Cc1ncsc1-c1ccc(CNC(=O)[C@@H]2C[C@@H](O)CN2C(=O)[C@@H](NC(=O)CCCCCCCNCCC(=O)N2CC(c3cc4cc(-c5ccccc5O)nnc4[nH]3)C2)C(C)(C)C)cc1] |
| 67 | 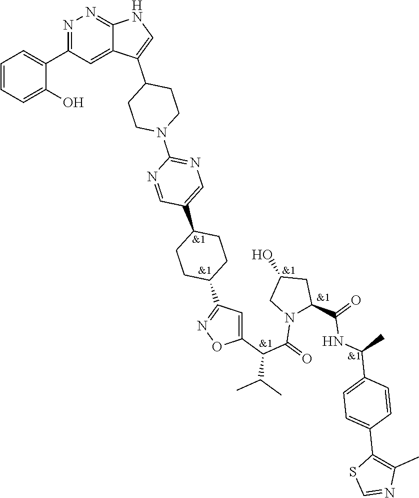 [SMILES: Cc1ncsc1-c1ccc([C@H](C)NC(=O)[C@@H]2C[C@@H](O)CN2C(=O)[C@@H](c2cc([C@H]3CC[C@H](c4cnc(N5CCC(c6c[nH]c7nnc(-c8ccccc8O)cc67)CC5)nc4)CC3)no2)C(C)C)cc1] |
| 68 | 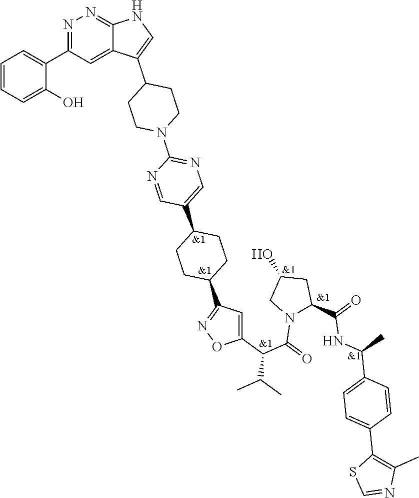 [SMILES: Cc1ncsc1-c1ccc([C@H](C)NC(=O)[C@@H]2C[C@@H](O)CN2C(=O)[C@@H](c2cc([C@H]3CC[C@@H](c4cnc(N5CCC(c6c[nH]c7nnc(-c8ccccc8O)cc67)CC5)nc4)CC3)no2)C(C)C)cc1] |
| 69 | 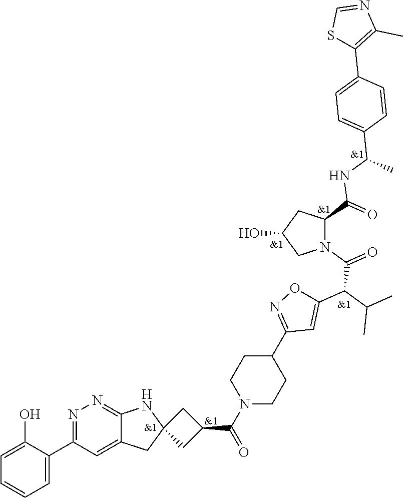 [SMILES: Cc1ncsc1-c1ccc([C@H](C)NC(=O)[C@@H]2C[C@@H](O)CN2C(=O)[C@@H](c2cc(C3CCN(C(=O)[C@H]4C[C@@]5(Cc6cc(-c7ccccc7O)nnc6N5)C4)CC3)no2)C(C)C)cc1] |
| 70 | 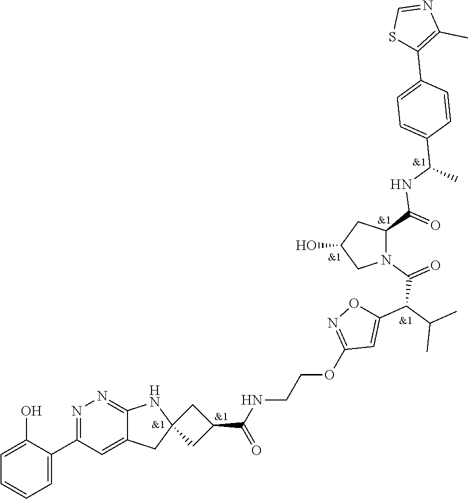 [SMILES: Cc1ncsc1-c1ccc([C@H](C)NC(=O)[C@@H]2C[C@@H](O)CN2C(=O)[C@@H](c2cc(OCCNC(=O)[C@H]3C[C@@]4(Cc5cc(-c6ccccc6O)nnc5N4)C3)no2)C(C)C)cc1] |
| 71 | 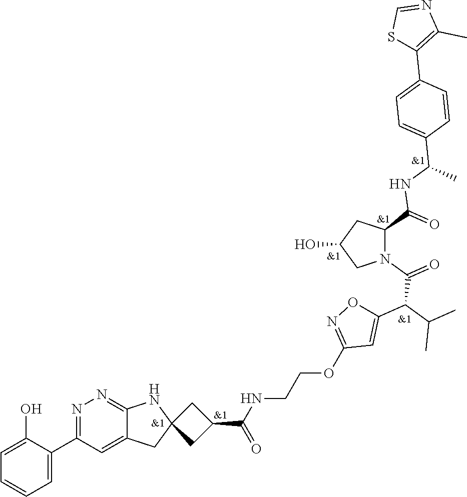 [SMILES: Cc1ncsc1-c1ccc([C@H](C)NC(=O)[C@@H]2C[C@@H](O)CN2C(=O)[C@@H](c2cc(OCCNC(=O)[C@H]3C[C@]4(Cc5cc(-c6ccccc6O)nnc5N4)C3)no2)C(C)C)cc1] |
| 72 | 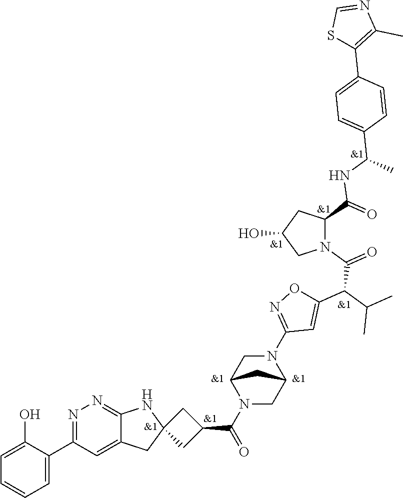 [SMILES: Cc1ncsc1-c1ccc([C@H](C)NC(=O)[C@@H]2C[C@@H](O)CN2C(=O)[C@@H](c2cc(N3C[C@@H]4C[C@H]3CN4C(=O)[C@H]3C[C@@]4(Cc5cc(-c6ccccc6O)nnc5N4)C3)no2)C(C)C)cc1] |
| 73 | 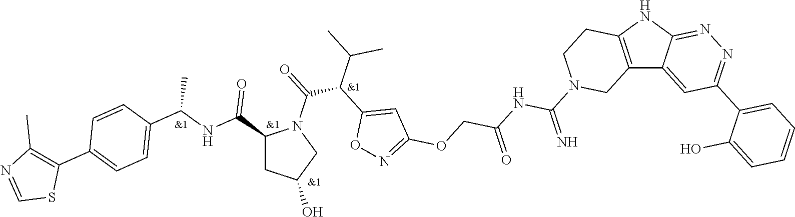 [SMILES: Cc1ncsc1-c1ccc([C@H](C)NC(=O)[C@@H]2C[C@@H](O)CN2C(=O)[C@@H](c2cc(OCC(=O)NC(=N)N3CCc4[nH]c5nnc(-c6ccccc6O)cc5c4C3)no2)C(C)C)cc1] |
| 74 | 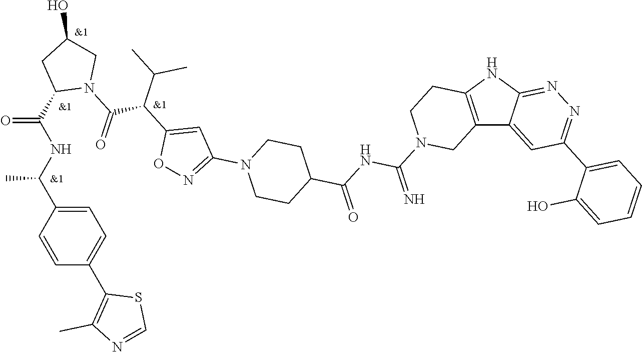 [SMILES: Cc1ncsc1-c1ccc([C@H](C)NC(=O)[C@@H]2C[C@@H](O)CN2C(=O)[C@@H](c2cc(N3CCC(C(=O)NC(=N)N4CCc5[nH]c6nnc(-c7ccccc7O)cc6c5C4)CC3)no2)C(C)C)cc1] |
| 75 | 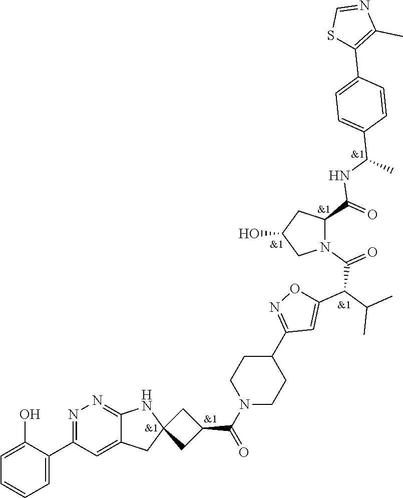 [SMILES: Cc1ncsc1-c1ccc([C@H](C)NC(=O)[C@@H]2C[C@@H](O)CN2C(=O)[C@@H](c2cc(C3CCN(C(=O)[C@H]4C[C@]5(Cc6cc(-c7ccccc7O)nnc6N5)C4)CC3)no2)C(C)C)cc1] |
| 76 | 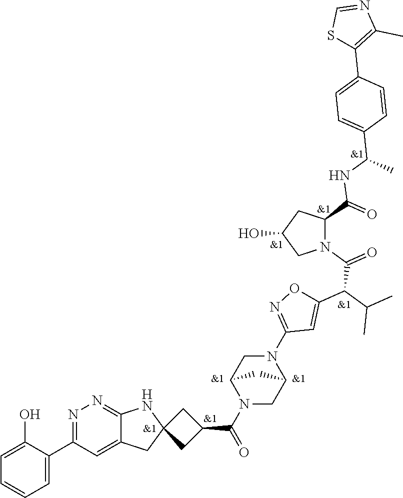 [SMILES: Cc1ncsc1-c1ccc([C@H](C)NC(=O)[C@@H]2C[C@@H](O)CN2C(=O)[C@@H](c2cc(N3C[C@H]4C[C@@H]3CN4C(=O)[C@H]3C[C@]4(Cc5cc(-c6ccccc6O)nnc5N4)C3)no2)C(C)C)cc1] |
| 77 | 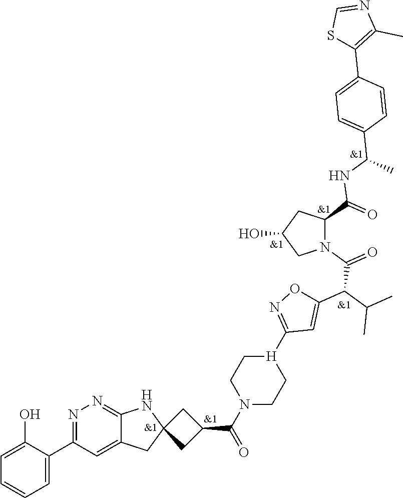 |
| 78 | 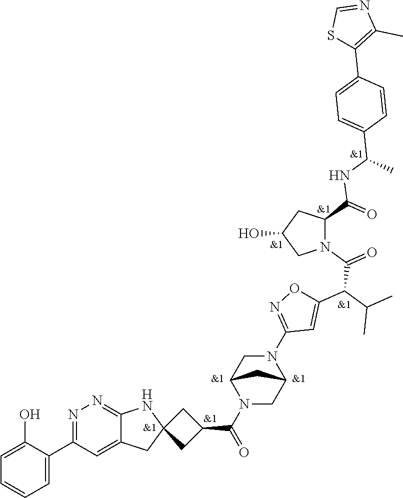 [SMILES: Cc1ncsc1-c1ccc([C@H](C)NC(=O)[C@@H]2C[C@@H](O)CN2C(=O)[C@@H](c2cc(N3C[C@@H]4C[C@H]3CN4C(=O)[C@H]3C[C@]4(Cc5cc(-c6ccccc6O)nnc5N4)C3)no2)C(C)C)cc1] |
| 79 | 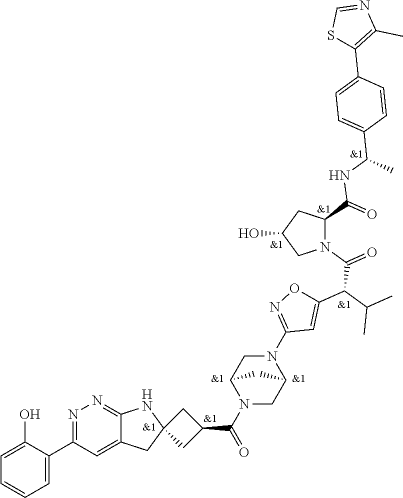 [SMILES: Cc1ncsc1-c1ccc([C@H](C)NC(=O)[C@@H]2C[C@@H](O)CN2C(=O)[C@@H](c2cc(N3C[C@H]4C[C@@H]3CN4C(=O)[C@H]3C[C@@]4(Cc5cc(-c6ccccc6O)nnc5N4)C3)no2)C(C)C)cc1] |
| 80 | 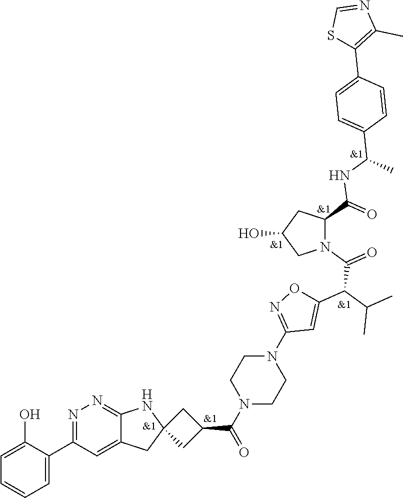 [SMILES: Cc1ncsc1-c1ccc([C@H](C)NC(=O)[C@@H]2C[C@@H](O)CN2C(=O)[C@@H](c2cc(N3CCN(C(=O)[C@H]4C[C@@]5(Cc6cc(-c7ccccc7O)nnc6N5)C4)CC3)no2)C(C)C)cc1] |
| 81 | 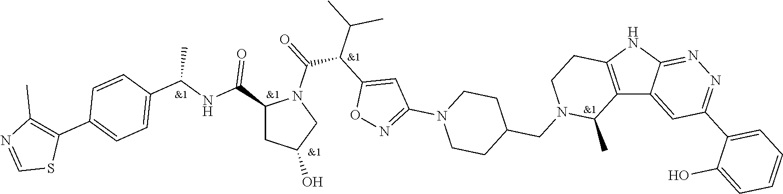 [SMILES: Cc1ncsc1-c1ccc([C@H](C)NC(=O)[C@@H]2C[C@@H](O)CN2C(=O)[C@@H](c2cc(N3CCC(CN4CCc5[nH]c6nnc(-c7ccccc7O)cc6c5[C@H]4C)CC3)no2)C(C)C)cc1] |
| 82 | 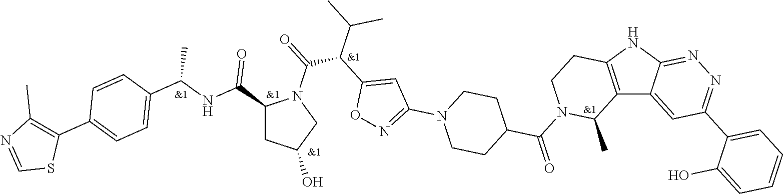 [SMILES: Cc1ncsc1-c1ccc([C@H](C)NC(=O)[C@@H]2C[C@@H](O)CN2C(=O)[C@@H](c2cc(N3CCC(C(=O)N4CCc5[nH]c6nnc(-c7ccccc7O)cc6c5[C@H]4C)CC3)no2)C(C)C)cc1] |
| 83 | 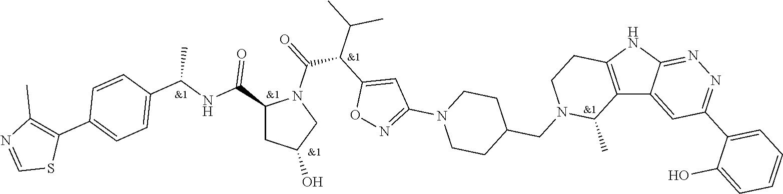 [SMILES: Cc1ncsc1-c1ccc([C@H](C)NC(=O)[C@@H]2C[C@@H](O)CN2C(=O)[C@@H](c2cc(N3CCC(CN4CCc5[nH]c6nnc(-c7ccccc7O)cc6c5[C@@H]4C)CC3)no2)C(C)C)cc1] |
| 84 | 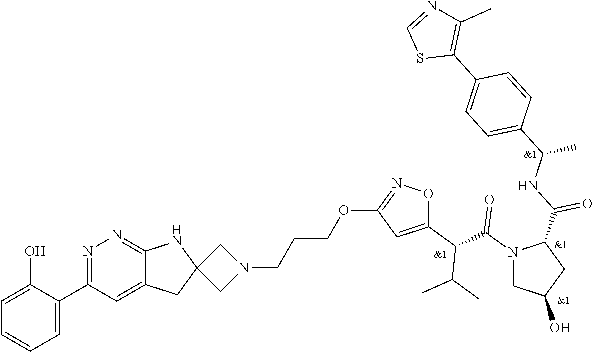 [SMILES: Cc1ncsc1-c1ccc([C@H](C)NC(=O)[C@@H]2C[C@@H](O)CN2C(=O)[C@@H](c2cc(OCCCN3CC4(Cc5cc(-c6ccccc6O)nnc5N4)C3)no2)C(C)C)cc1] |
| 85 | 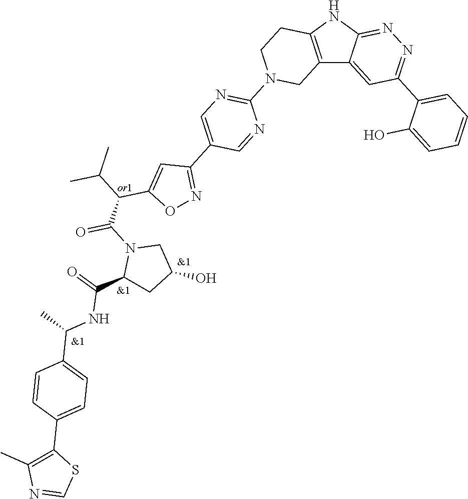 [SMILES: Cc1ncsc1-c1ccc([C@H](C)NC(=O)[C@@H]2C[C@@H](O)CN2C(=O)[C@@H](c2cc(-c3cnc(N4CCc5[nH]c6nnc(-c7ccccc7O)cc6c5C4)nc3)no2)C(C)C)cc1] |
| 86 | 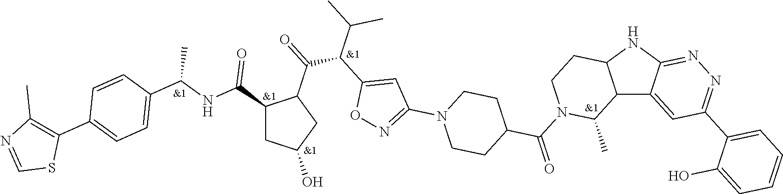 [SMILES: Cc1ncsc1-c1ccc([C@H](C)NC(=O)[C@@H]2C[C@@H](O)CC2C(=O)[C@@H](c2cc(N3CCC(C(=O)N4CCC5Nc6nnc(-c7ccccc7O)cc6C5[C@@H]4C)CC3)no2)C(C)C)cc1] |
| 87 | 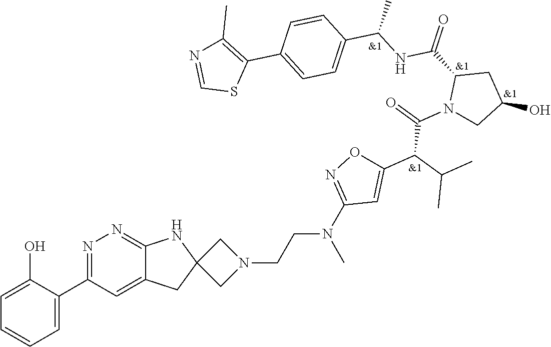 [SMILES: Cc1ncsc1-c1ccc([C@H](C)NC(=O)[C@@H]2C[C@@H](O)CN2C(=O)[C@@H](c2cc(N(C)CCN3CC4(Cc5cc(-c6ccccc6O)nnc5N4)C3)no2)C(C)C)cc1] |
| 88 | 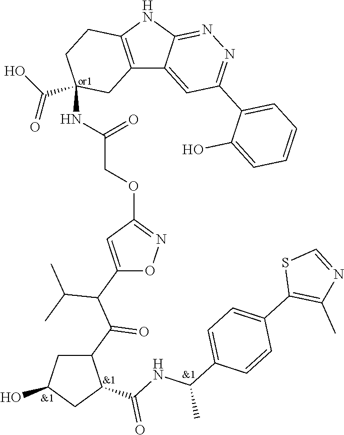 [SMILES: Cc1ncsc1-c1ccc([C@H](C)NC(=O)[C@@H]2C[C@@H](O)CC2C(=O)C(c2cc(OCC(=O)N[C@@]3(C(=O)O)CCc4[nH]c5nnc(-c6ccccc6O)cc5c4C3)no2)C(C)C)cc1] |
| 89 | 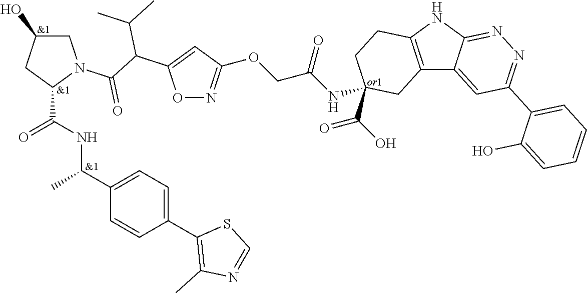 [SMILES: Cc1ncsc1-c1ccc([C@H](C)NC(=O)[C@@H]2C[C@@H](O)CN2C(=O)C(c2cc(OCC(=O)N[C@]3(C(=O)O)CCc4[nH]c5nnc(-c6ccccc6O)cc5c4C3)no2)C(C)C)cc1] |
| 90 | 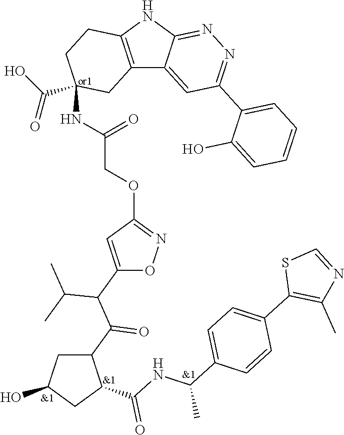 [SMILES: Cc1ncsc1-c1ccc([C@H](C)NC(=O)[C@@H]2C[C@@H](O)CC2C(=O)C(c2cc(OCC(=O)N[C@@]3(C(=O)O)CCc4[nH]c5nnc(-c6ccccc6O)cc5c4C3)no2)C(C)C)cc1] |
| 91 | 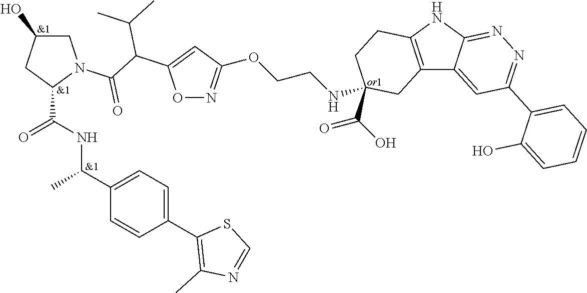 [SMILES: Cc1ncsc1-c1ccc([C@H](C)NC(=O)[C@@H]2C[C@@H](O)CN2C(=O)C(c2cc(OCCN[C@]3(C(=O)O)CCc4[nH]c5nnc(-c6ccccc6O)cc5c4C3)no2)C(C)C)cc1] |
| 92 | 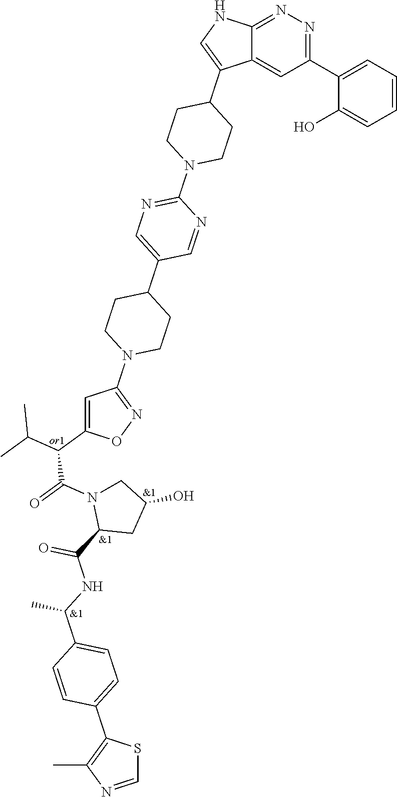 [SMILES: Cc1ncsc1-c1ccc([C@H](C)NC(=O)[C@@H]2C[C@@H](O)CN2C(=O)[C@@H](c2cc(N3CCC(c4cnc(N5CCC(c6c[nH]c7nnc(-c8ccccc8O)cc67)CC5)nc4)CC3)no2)C(C)C)cc1] |
| 93 | 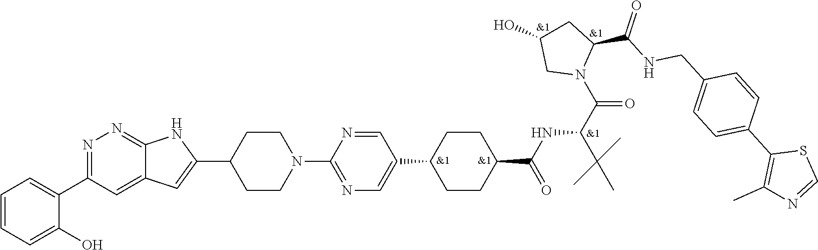 [SMILES: Cc1ncsc1-c1ccc(CNC(=O)[C@@H]2C[C@@H](O)CN2C(=O)[C@@H](NC(=O)[C@H]2CC[C@H](c3cnc(N4CCC(c5cc6cc(-c7ccccc7O)nnc6[nH]5)CC4)nc3)CC2)C(C)(C)C)cc1] |
| 94 | 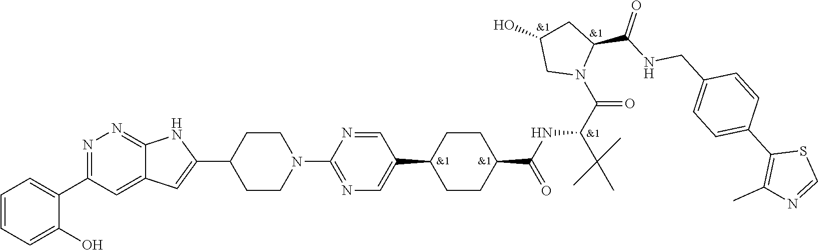 [SMILES: Cc1ncsc1-c1ccc(CNC(=O)[C@@H]2C[C@@H](O)CN2C(=O)[C@@H](NC(=O)[C@H]2CC[C@@H](c3cnc(N4CCC(c5cc6cc(-c7ccccc7O)nnc6[nH]5)CC4)nc3)CC2)C(C)(C)C)cc1] |
| 95 | 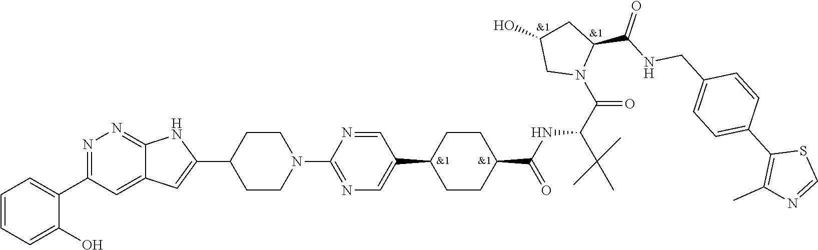 [SMILES: Cc1ncsc1-c1ccc(CNC(=O)[C@@H]2C[C@@H](O)CN2C(=O)[C@@H](NC(=O)[C@H]2CC[C@@H](c3cnc(N4CCC(c5cc6cc(-c7ccccc7O)nnc6[nH]5)CC4)nc3)CC2)C(C)(C)C)cc1] |
| 96 | 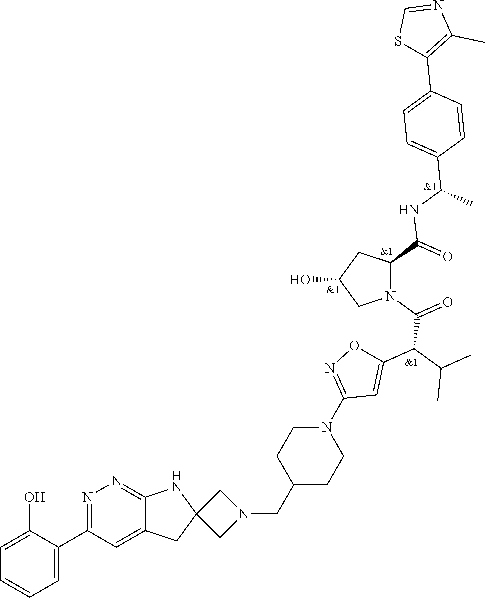 [SMILES: Cc1ncsc1-c1ccc([C@H](C)NC(=O)[C@@H]2C[C@@H](O)CN2C(=O)[C@@H](c2cc(N3CCC(CN4CC5(Cc6cc(-c7ccccc7O)nnc6N5)C4)CC3)no2)C(C)C)cc1] |
| 97 | 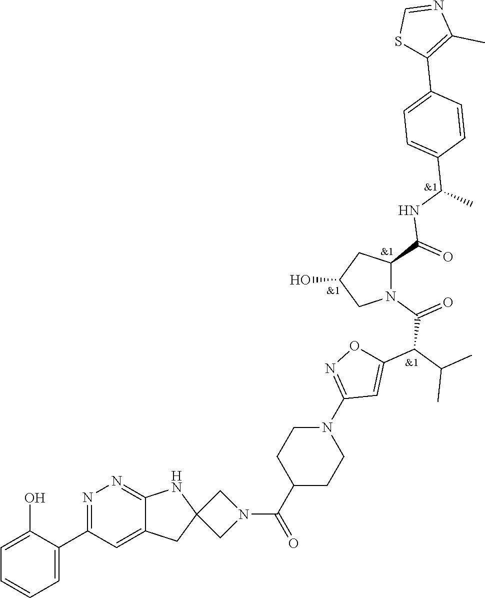 [SMILES: Cc1ncsc1-c1ccc([C@H](C)NC(=O)[C@@H]2C[C@@H](O)CN2C(=O)[C@@H](c2cc(N3CCC(C(=O)N4CC5(Cc6cc(-c7ccccc7O)nnc6N5)C4)CC3)no2)C(C)C)cc1] |
| 98 | 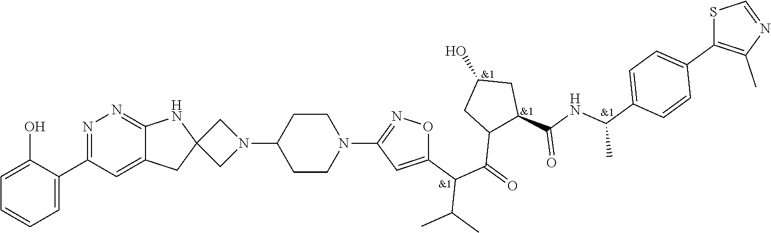 [SMILES: Cc1ncsc1-c1ccc([C@H](C)NC(=O)[C@@H]2C[C@@H](O)CC2C(=O)C(c2cc(N3CCC(N4CC5(Cc6cc(-c7ccccc7O)nnc6N5)C4)CC3)no2)C(C)C)cc1] |
| 99 | 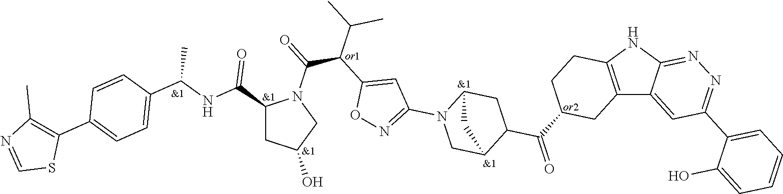 [SMILES: Cc1ncsc1-c1ccc([C@H](C)NC(=O)[C@@H]2C[C@@H](O)CN2C(=O)[C@H](c2cc(N3C[C@H]4C[C@@H]3CC4C(=O)[C@@H]3CCc4[nH]c5nnc(-c6ccccc6O)cc5c4C3)no2)C(C)C)cc1] |
| 100 | 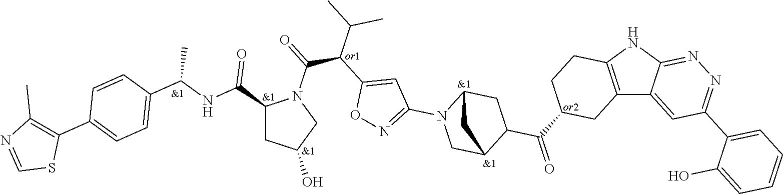 [SMILES: Cc1ncsc1-c1ccc([C@H](C)NC(=O)[C@@H]2C[C@@H](O)CN2C(=O)[C@H](c2cc(N3C[C@@H]4C[C@H]3CC4C(=O)[C@@H]3CCc4[nH]c5nnc(-c6ccccc6O)cc5c4C3)no2)C(C)C)cc1] |
| 101 | 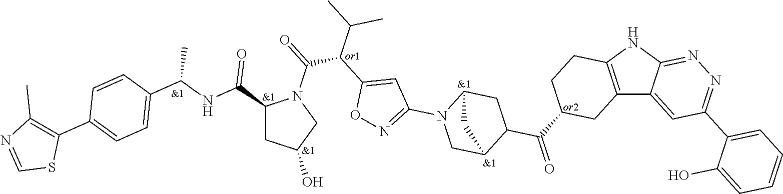 [SMILES: Cc1ncsc1-c1ccc([C@H](C)NC(=O)[C@@H]2C[C@@H](O)CN2C(=O)[C@@H](c2cc(N3C[C@H]4C[C@@H]3CC4C(=O)[C@@H]3CCc4[nH]c5nnc(-c6ccccc6O)cc5c4C3)no2)C(C)C)cc1] |
| 102 | 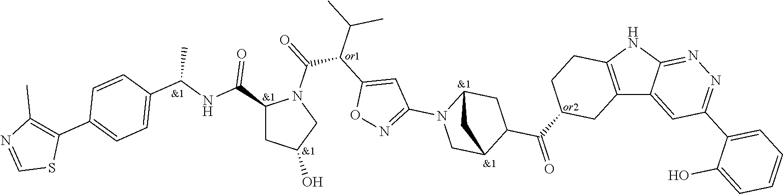 [SMILES: Cc1ncsc1-c1ccc([C@H](C)NC(=O)[C@@H]2C[C@@H](O)CN2C(=O)[C@@H](c2cc(N3C[C@@H]4C[C@H]3CC4C(=O)[C@@H]3CCc4[nH]c5nnc(-c6ccccc6O)cc5c4C3)no2)C(C)C)cc1] |
| 103 | 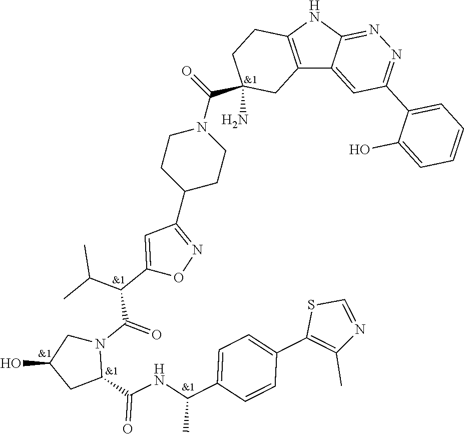 [SMILES: Cc1ncsc1-c1ccc([C@H](C)NC(=O)[C@@H]2C[C@@H](O)CN2C(=O)[C@@H](c2cc(C3CCN(C(=O)[C@@]4(N)CCc5[nH]c6nnc(-c7ccccc7O)cc6c5C4)CC3)no2)C(C)C)cc1] |
| 104 | 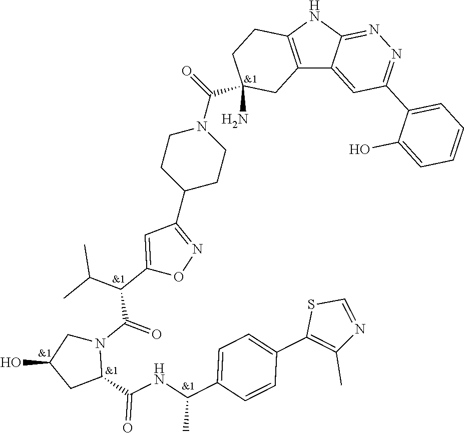 [SMILES: Cc1ncsc1-c1ccc([C@H](C)NC(=O)[C@@H]2C[C@@H](O)CN2C(=O)[C@@H](c2cc(C3CCN(C(=O)[C@]4(N)CCc5[nH]c6nnc(-c7ccccc7O)cc6c5C4)CC3)no2)C(C)C)cc1] |
| 105 | 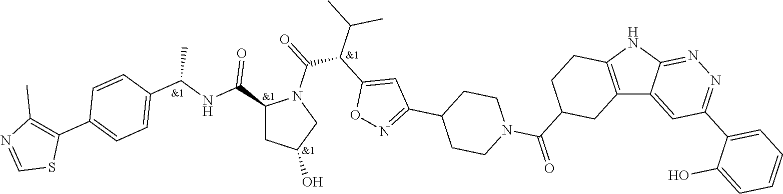 [SMILES: Cc1ncsc1-c1ccc([C@H](C)NC(=O)[C@@H]2C[C@@H](O)CN2C(=O)[C@@H](c2cc(C3CCN(C(=O)C4CCc5[nH]c6nnc(-c7ccccc7O)cc6c5C4)CC3)no2)C(C)C)cc1] |
| 106 | 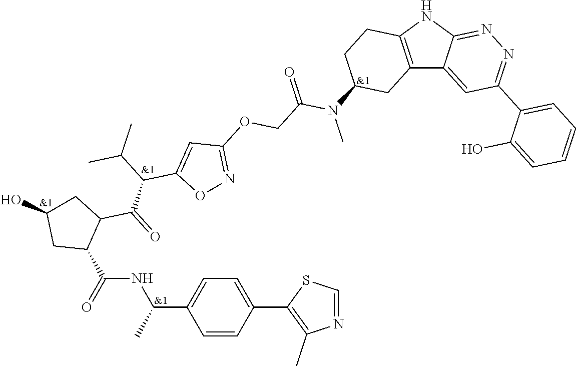 [SMILES: Cc1ncsc1-c1ccc([C@H](C)NC(=O)[C@@H]2C[C@@H](O)CC2C(=O)[C@@H](c2cc(OCC(=O)N(C)[C@H]3CCc4[nH]c5nnc(-c6ccccc6O)cc5c4C3)no2)C(C)C)cc1] |
| 107 | 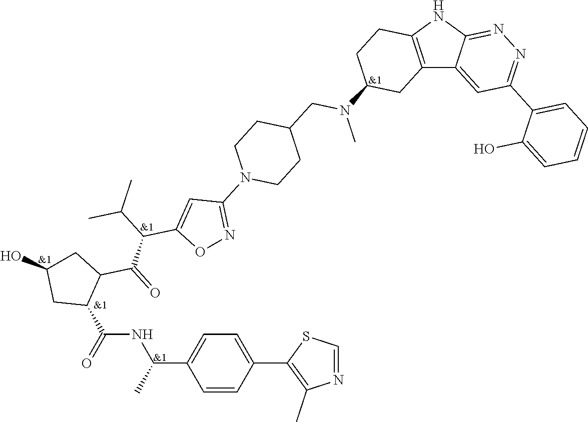 [SMILES: Cc1ncsc1-c1ccc([C@H](C)NC(=O)[C@@H]2C[C@@H](O)CC2C(=O)[C@@H](c2cc(N3CCC(CN(C)[C@H]4CCc5[nH]c6nnc(-c7ccccc7O)cc6c5C4)CC3)no2)C(C)C)cc1] |
| 108 | 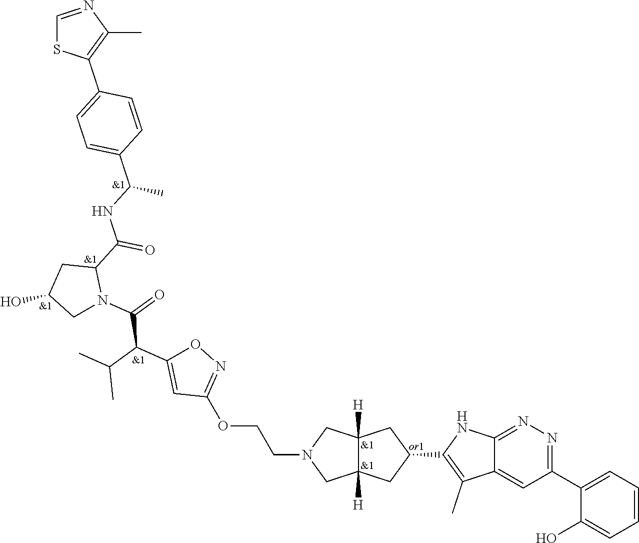 [SMILES: Cc1ncsc1-c1ccc([C@H](C)NC(=O)C2C[C@@H](O)CN2C(=O)[C@@H](c2cc(OCCN3C[C@H]4C[C@@H](c5[nH]c6nnc(-c7ccccc7O)cc6c5C)C[C@H]4C3)no2)C(C)C)cc1] |
| 109 | 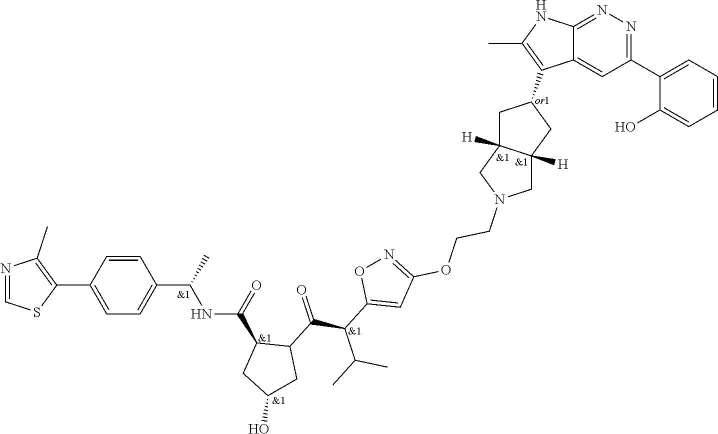 [SMILES: Cc1ncsc1-c1ccc([C@H](C)NC(=O)[C@@H]2C[C@@H](O)CC2C(=O)[C@@H](c2cc(OCCN3C[C@H]4C[C@@H](c5c(C)[nH]c6nnc(-c7ccccc7O)cc56)C[C@H]4C3)no2)C(C)C)cc1] |
| 110 | 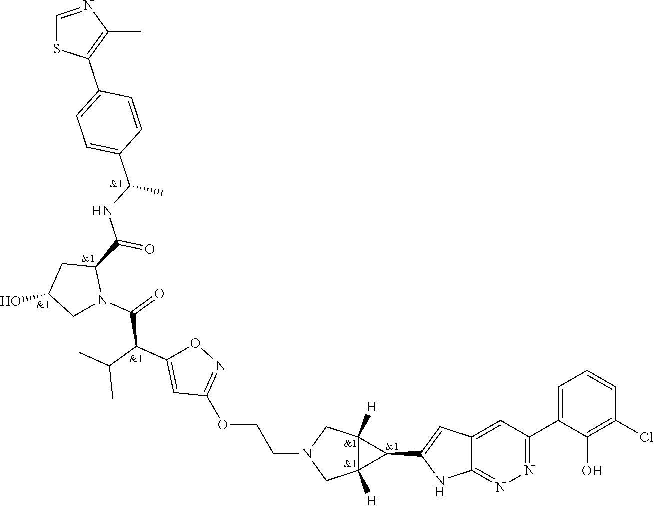 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3cc4cc(-c5cccc(Cl)c5O)nnc4[nH]3)no2)C(C)C)cc1] |
| 111 | 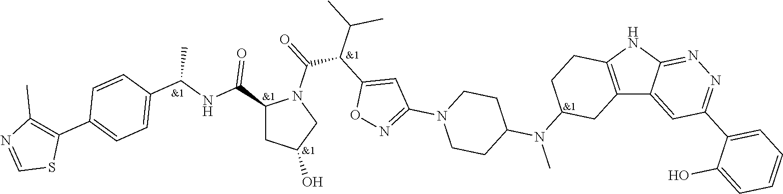 [SMILES: Cc1ncsc1-c1ccc([C@H](C)NC(=O)[C@@H]2C[C@@H](O)CN2C(=O)[C@@H](c2cc(N3CCC(N(C)C4CCc5[nH]c6nnc(-c7ccccc7O)cc6c5C4)CC3)no2)C(C)C)cc1] |
| 112 | 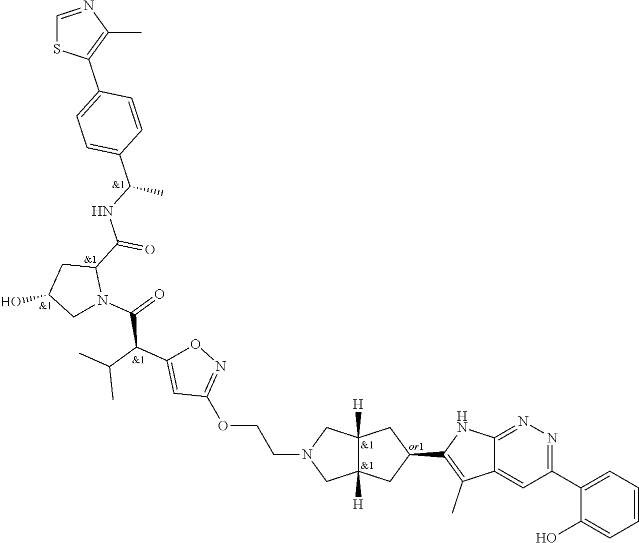 [SMILES: Cc1ncsc1-c1ccc([C@H](C)NC(=O)C2C[C@@H](O)CN2C(=O)[C@@H](c2cc(OCCN3C[C@H]4C[C@H](c5[nH]c6nnc(-c7ccccc7O)cc6c5C)C[C@H]4C3)no2)C(C)C)cc1] |
| 113 | 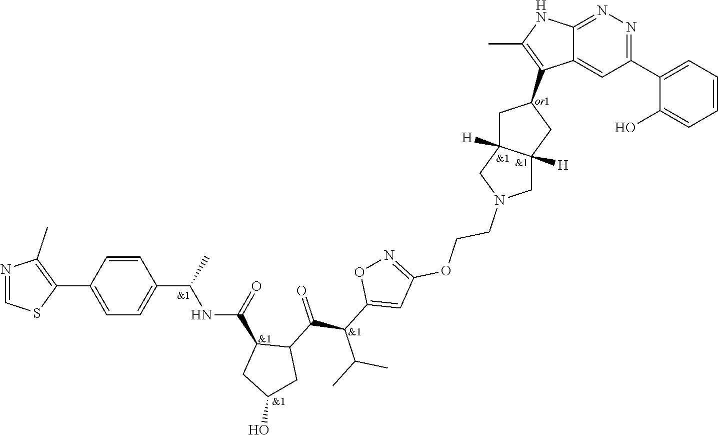 [SMILES: Cc1ncsc1-c1ccc([C@H](C)NC(=O)[C@@H]2C[C@@H](O)CC2C(=O)[C@@H](c2cc(OCCN3C[C@H]4C[C@H](c5c(C)[nH]c6nnc(-c7ccccc7O)cc56)C[C@H]4C3)no2)C(C)C)cc1] |
| 114 | 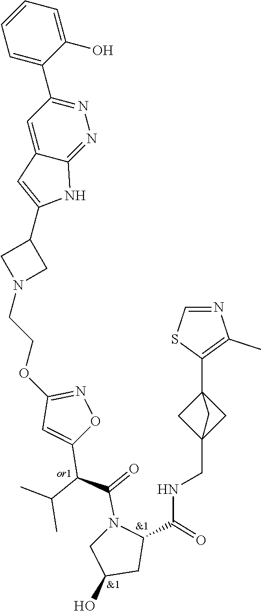 [SMILES: Cc1ncsc1C12CC(CNC(=O)[C@@H]3C[C@@H](O)CN3C(=O)[C@H](c3cc(OCCN4CC(c5cc6cc(-c7ccccc7O)nnc6[nH]5)C4)no3)C(C)C)(C1)C2] |
| 115 | 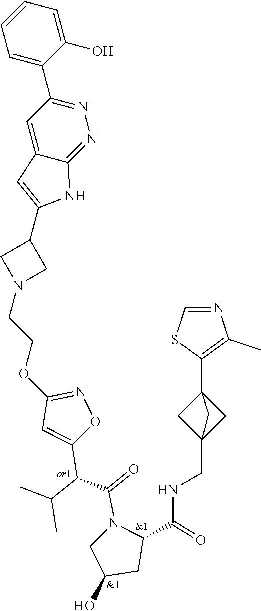 [SMILES: Cc1ncsc1C12CC(CNC(=O)[C@@H]3C[C@@H](O)CN3C(=O)[C@@H](c3cc(OCCN4CC(c5cc6cc(-c7ccccc7O)nnc6[nH]5)C4)no3)C(C)C)(C1)C2] |
| 116 | 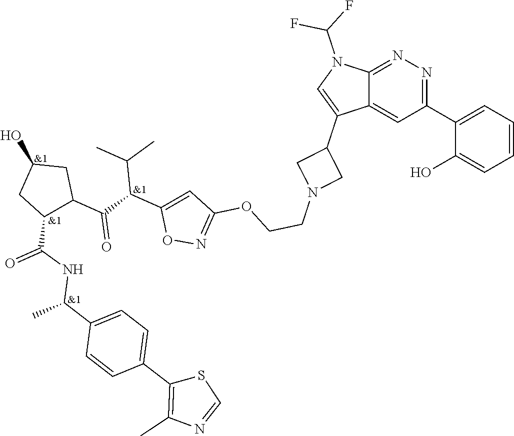 [SMILES: Cc1ncsc1-c1ccc([C@H](C)NC(=O)[C@@H]2C[C@@H](O)CC2C(=O)[C@@H](c2cc(OCCN3CC(c4cn(C(F)F)c5nnc(-c6ccccc6O)cc45)C3)no2)C(C)C)cc1] |
| 117 | 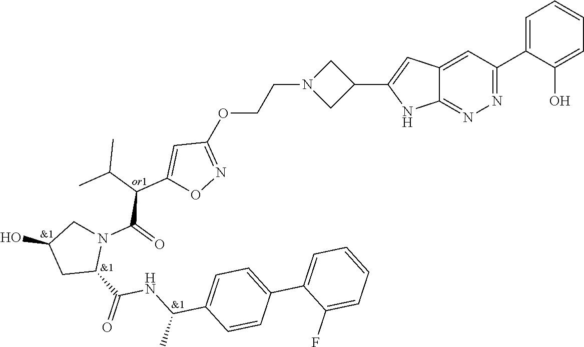 [SMILES: CC(C)[C@H](C(=O)N1C[C@H](O)C[C@H]1C(=O)N[C@@H](C)c1ccc(-c2ccccc2F)cc1)c1cc(OCCN2CC(c3cc4cc(-c5ccccc5O)nnc4[nH]3)C2)no1] |
| 118 | 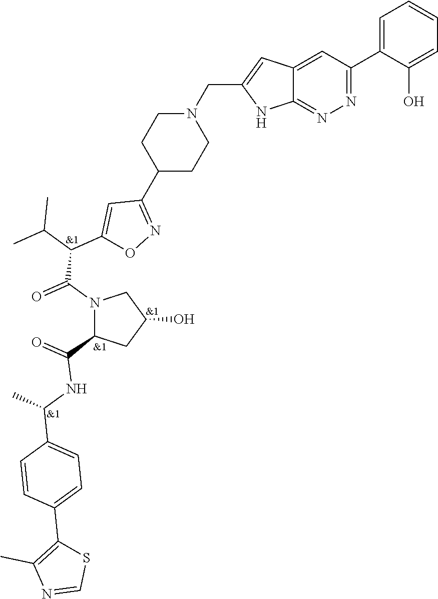 [SMILES: Cc1ncsc1-c1ccc([C@H](C)NC(=O)[C@@H]2C[C@@H](O)CN2C(=O)[C@@H](c2cc(C3CCN(Cc4cc5cc(-c6ccccc6O)nnc5[nH]4)CC3)no2)C(C)C)cc1] |
| 119 | 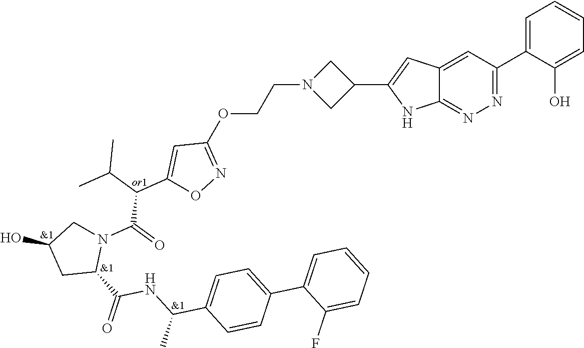 [SMILES: CC(C)[C@@H](C(=O)N1C[C@H](O)C[C@H]1C(=O)N[C@@H](C)c1ccc(-c2ccccc2F)cc1)c1cc(OCCN2CC(c3cc4cc(-c5ccccc5O)nnc4[nH]3)C2)no1] |
| 120 | 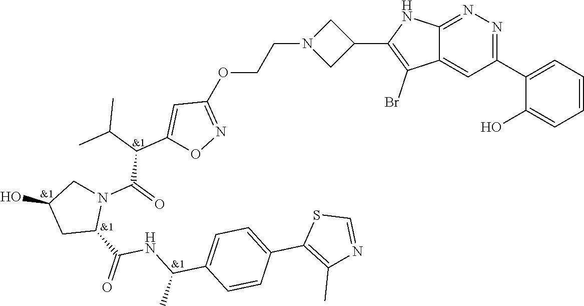 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6ccccc6O)cc5c4Br)C3)no2)C(C)C)cc1] |
| 121 | 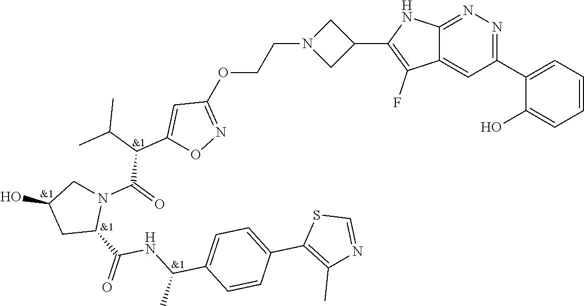 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6ccccc6O)cc5c4F)C3)no2)C(C)C)cc1] |
| 122 | 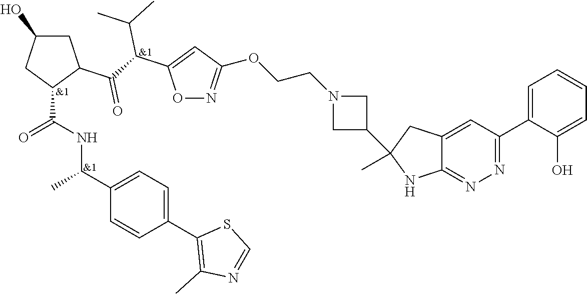 [SMILES: Cc1ncsc1-c1ccc([C@H](C)NC(=O)[C@@H]2C[C@@H](O)CC2C(=O)[C@@H](c2cc(OCCN3CC(C4(C)Cc5cc(-c6ccccc6O)nnc5N4)C3)no2)C(C)C)cc1] |
| 123 | 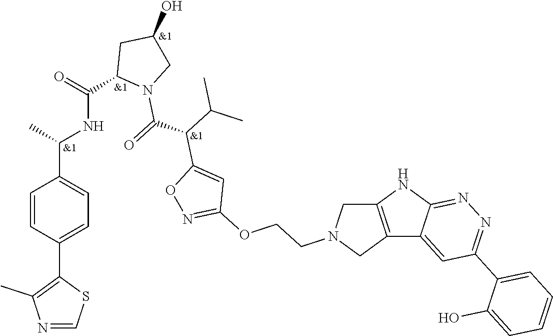 [SMILES: Cc1ncsc1-c1ccc([C@H](C)NC(=O)[C@@H]2C[C@@H](O)CN2C(=O)[C@@H](c2cc(OCCN3Cc4[nH]c5nnc(-c6ccccc6O)cc5c4C3)no2)C(C)C)cc1] |
| 124 | 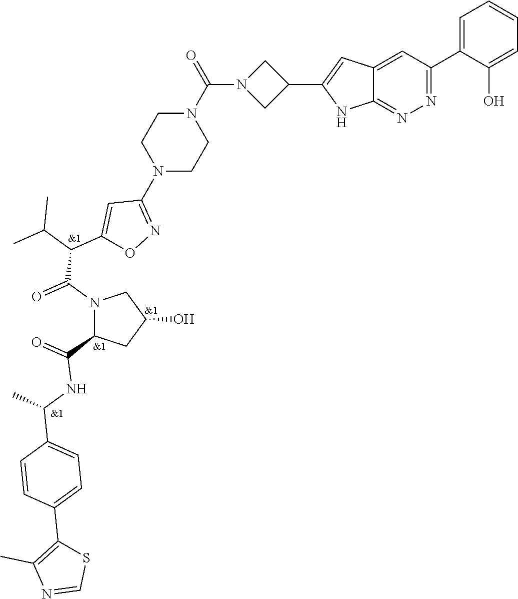 [SMILES: Cc1ncsc1-c1ccc([C@H](C)NC(=O)[C@@H]2C[C@@H](O)CN2C(=O)[C@@H](c2cc(N3CCN(C(=O)N4CC(c5cc6cc(-c7ccccc7O)nnc6[nH]5)C4)CC3)no2)C(C)C)cc1] |
| 125 | 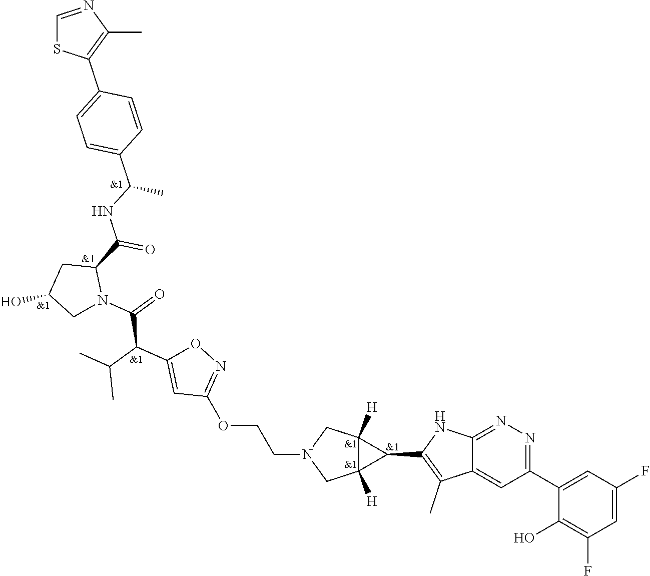 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3[nH]c4nnc(-c5cc(F)cc(F)c5O)cc4c3C)no2)C(C)C)cc1] |
| 126 | 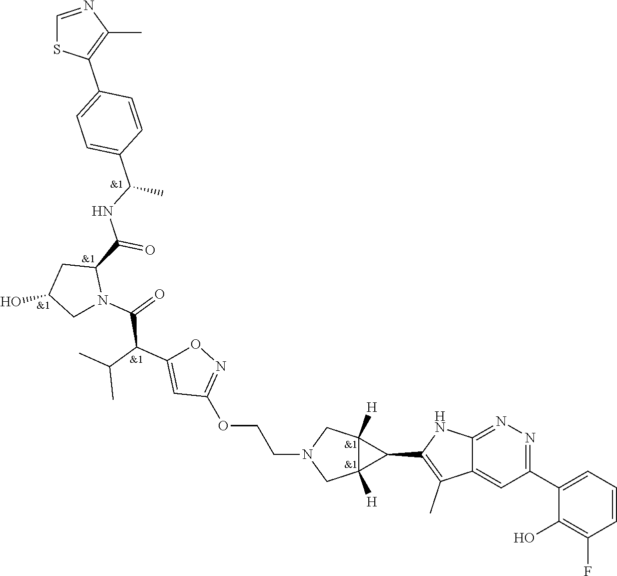 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3[nH]c4nnc(-c5cccc(F)c5O)cc4c3C)no2)C(C)C)cc1] |
| 127 | 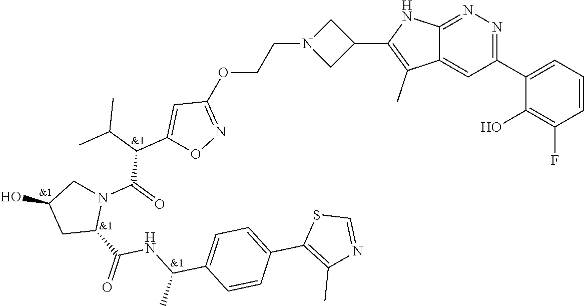 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6cccc(F)c6O)cc5c4C)C3)no2)C(C)C)cc1] |
| 128 | 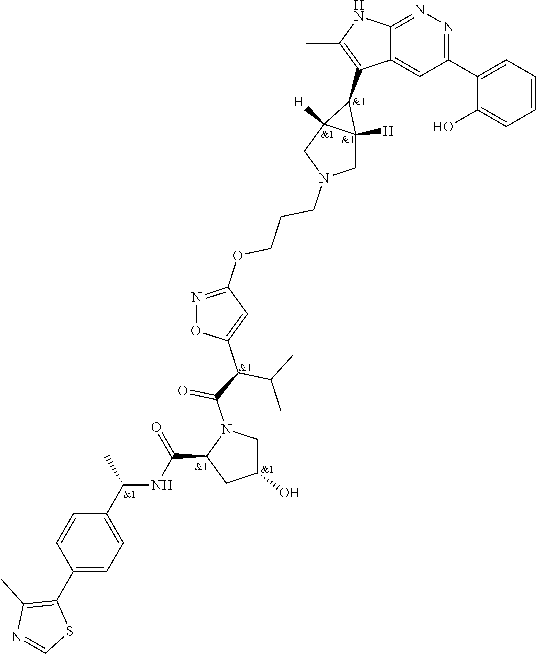 [SMILES: Cc1ncsc1-c1ccc([C@H](C)NC(=O)[C@@H]2C[C@@H](O)CN2C(=O)[C@@H](c2cc(OCCCN3C[C@@H]4[C@H](C3)[C@H]4c3c(C)[nH]c4nnc(-c5ccccc5O)cc34)no2)C(C)C)cc1] |
| 129 | 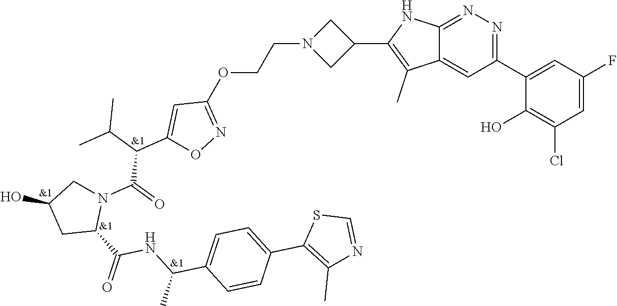 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6cc(F)cc(Cl)c6O)cc5c4C)C3)no2)C(C)C)cc1] |
| 130 | 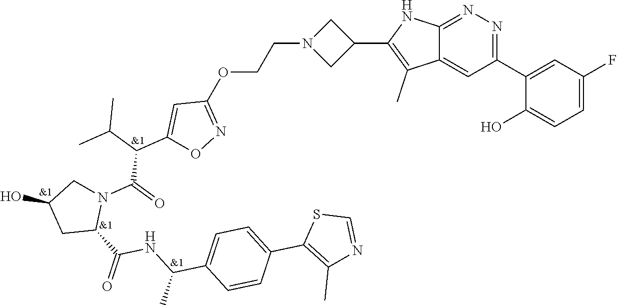 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6cc(F)ccc6O)cc5c4C)C3)no2)C(C)C)cc1] |
| 131 | 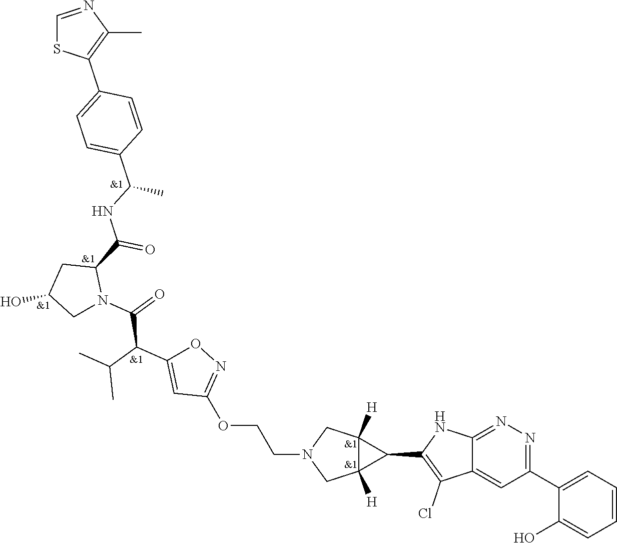 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3[nH]c4nnc(-c5ccccc5O)cc4c3Cl)no2)C(C)C)cc1] |
| 132 | 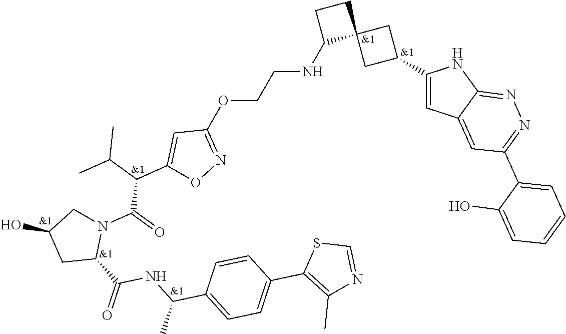 |
| 133 | 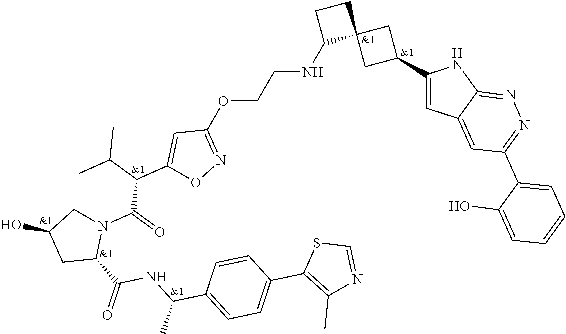 |
| 134 | 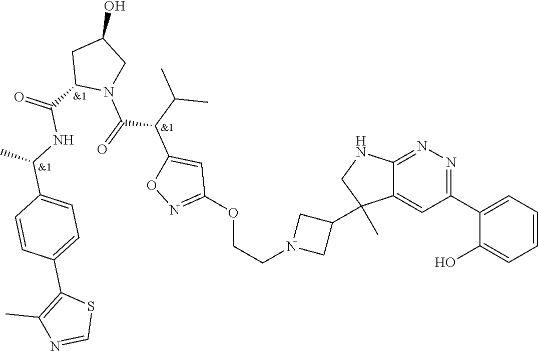 [SMILES: Cc1ncsc1-c1ccc([C@H](C)NC(=O)[C@@H]2C[C@@H](O)CN2C(=O)[C@@H](c2cc(OCCN3CC(C4(C)CNc5nnc(-c6ccccc6O)cc54)C3)no2)C(C)C)cc1] |
| 135 | 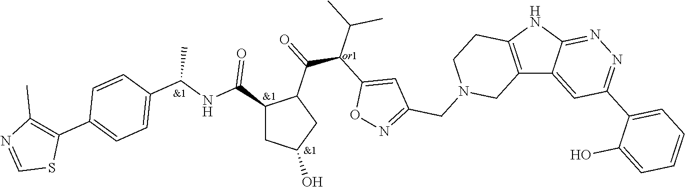 [SMILES: Cc1ncsc1-c1ccc([C@H](C)NC(=O)[C@@H]2C[C@@H](O)CC2C(=O)[C@H](c2cc(CN3CCc4[nH]c5nnc(-c6ccccc6O)cc5c4C3)no2)C(C)C)cc1] |
| 136 | 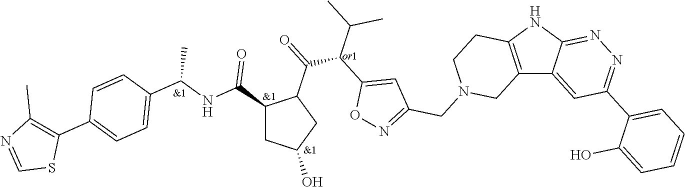 [SMILES: Cc1ncsc1-c1ccc([C@H](C)NC(=O)[C@@H]2C[C@@H](O)CC2C(=O)[C@@H](c2cc(CN3CCc4[nH]c5nnc(-c6ccccc6O)cc5c4C3)no2)C(C)C)cc1] |
| 137 | 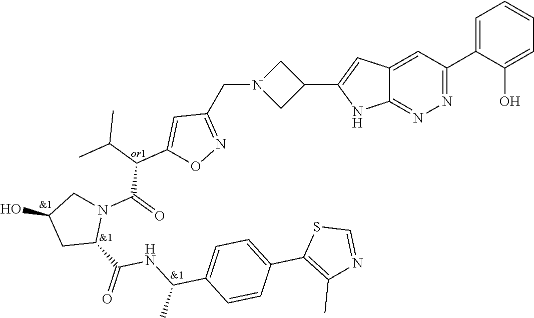 [SMILES: Cc1ncsc1-c1ccc([C@H](C)NC(=O)[C@@H]2C[C@@H](O)CN2C(=O)[C@@H](c2cc(CN3CC(c4cc5cc(-c6ccccc6O)nnc5[nH]4)C3)no2)C(C)C)cc1] |
| 138 | 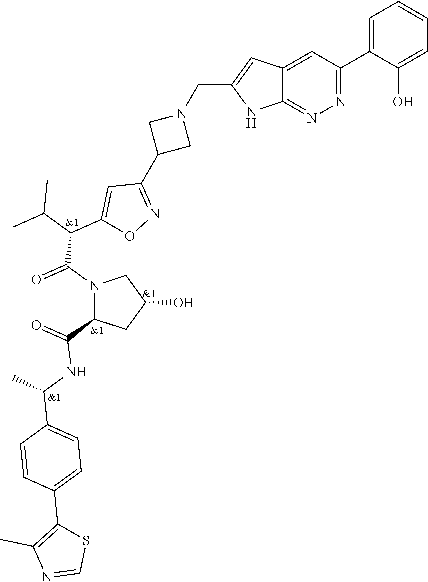 [SMILES: Cc1ncsc1-c1ccc([C@H](C)NC(=O)[C@@H]2C[C@@H](O)CN2C(=O)[C@@H](c2cc(C3CN(Cc4cc5cc(-c6ccccc6O)nnc5[nH]4)C3)no2)C(C)C)cc1] |
| 139 | 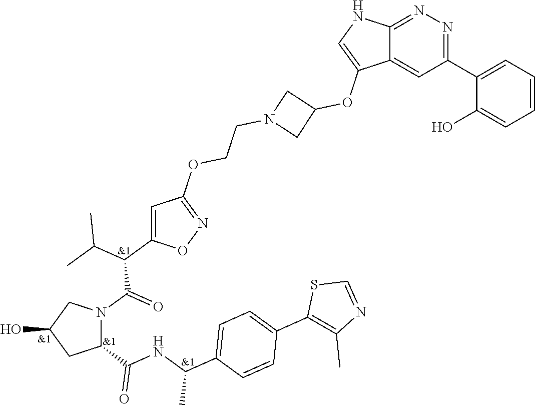 [SMILES: Cc1ncsc1-c1ccc([C@H](C)NC(=O)[C@@H]2C[C@@H](O)CN2C(=O)[C@@H](c2cc(OCCN3CC(Oc4c[nH]c5nnc(-c6ccccc6O)cc45)C3)no2)C(C)C)cc1] |
| 140 | 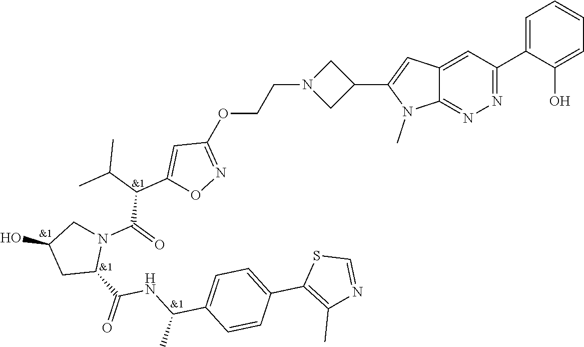 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5n4C)C3)no2)C(C)C)cc1] |
| 141 | 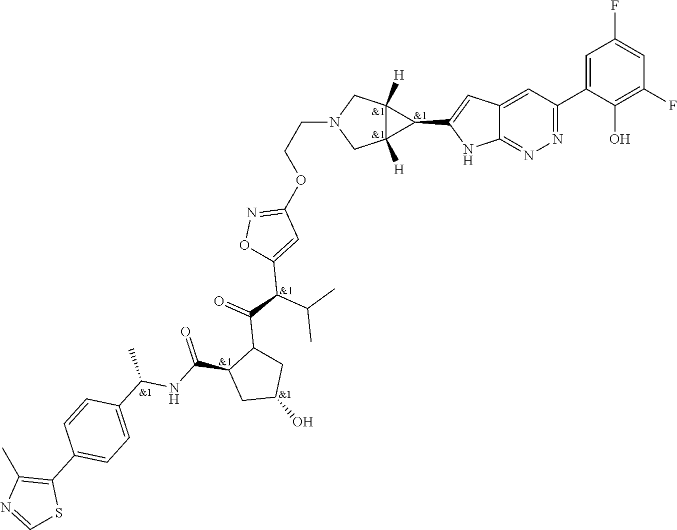 [SMILES: Cc1ncsc1-c1ccc([C@H](C)NC(=O)[C@@H]2C[C@@H](O)CC2C(=O)[C@@H](c2cc(OCCN3C[C@@H]4[C@H](C3)[C@H]4c3cc4cc(-c5cc(F)cc(F)c5O)nnc4[nH]3)no2)C(C)C)cc1] |
| 142 | 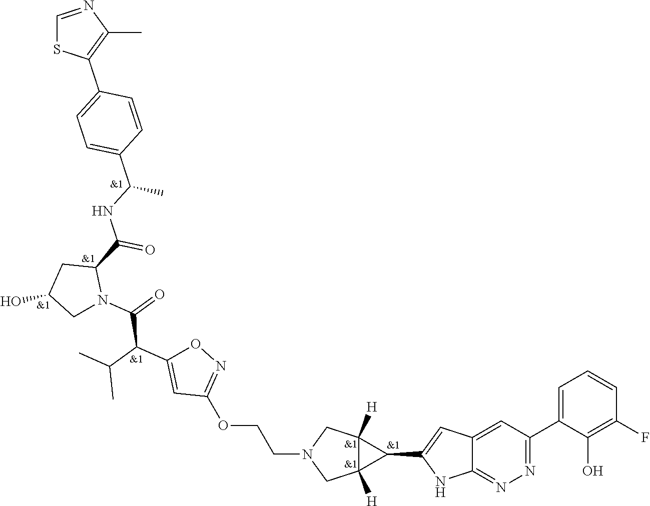 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3cc4cc(-c5cccc(F)c5O)nnc4[nH]3)no2)C(C)C)cc1] |
| 143 | 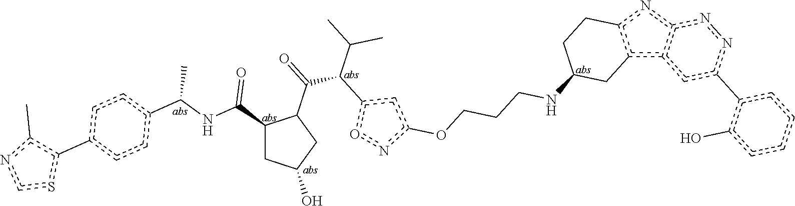 |
| 144 | 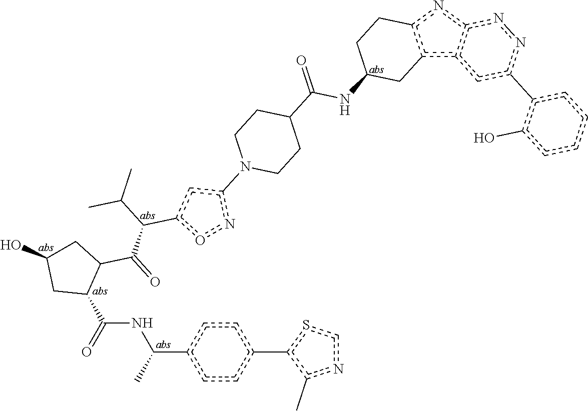 |
| 145 | 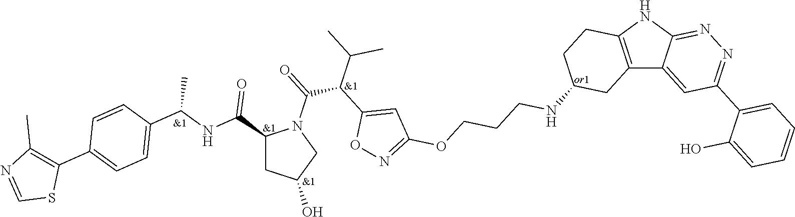 [SMILES: Cc1ncsc1-c1ccc([C@H](C)NC(=O)[C@@H]2C[C@@H](O)CN2C(=O)[C@@H](c2cc(OCCCN[C@@H]3CCc4[nH]c5nnc(-c6ccccc6O)cc5c4C3)no2)C(C)C)cc1] |
| 146 | 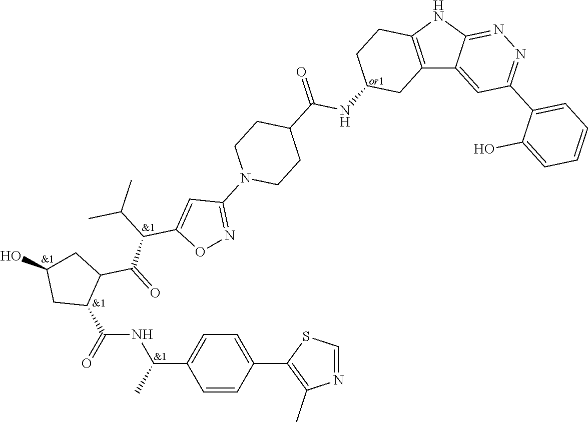 [SMILES: Cc1ncsc1-c1ccc([C@H](C)NC(=O)[C@@H]2C[C@@H](O)CC2C(=O)[C@@H](c2cc(N3CCC(C(=O)N[C@@H]4CCc5[nH]c6nnc(-c7ccccc7O)cc6c5C4)CC3)no2)C(C)C)cc1] |
| 147 | 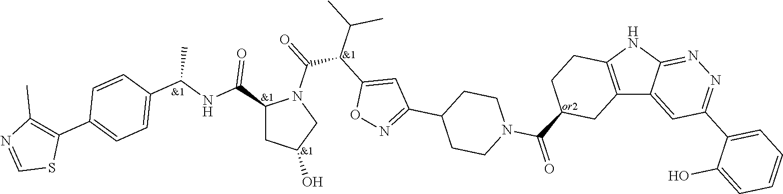 [SMILES: Cc1ncsc1-c1ccc([C@H](C)NC(=O)[C@@H]2C[C@@H](O)CN2C(=O)[C@@H](c2cc(C3CCN(C(=O)[C@H]4CCc5[nH]c6nnc(-c7ccccc7O)cc6c5C4)CC3)no2)C(C)C)cc1] |
| 148 | 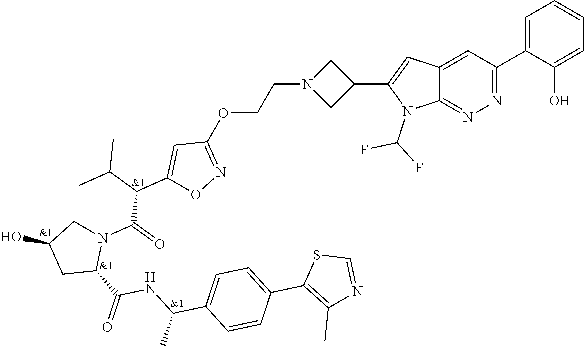 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5n4C(F)F)C3)no2)C(C)C)cc1] |
| 149 | 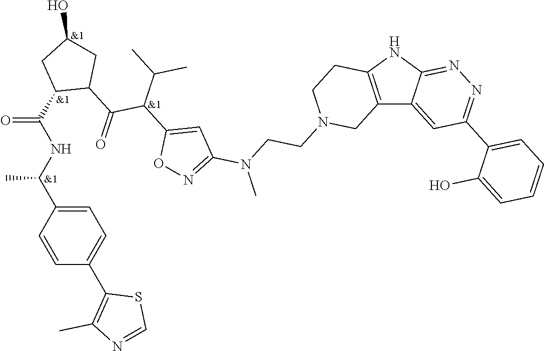 [SMILES: Cc1ncsc1-c1ccc([C@H](C)NC(=O)[C@@H]2C[C@@H](O)CC2C(=O)C(c2cc(N(C)CCN3CCc4[nH]c5nnc(-c6ccccc6O)cc5c4C3)no2)C(C)C)cc1] |
| 150 | 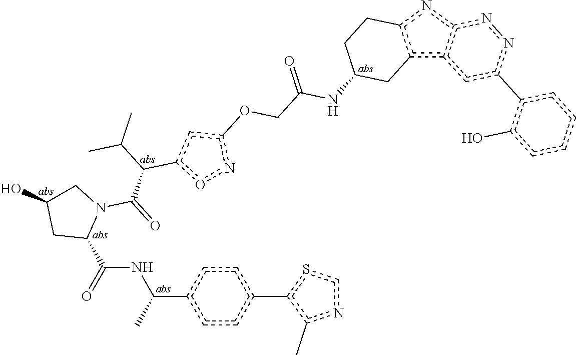 |
| 151 | 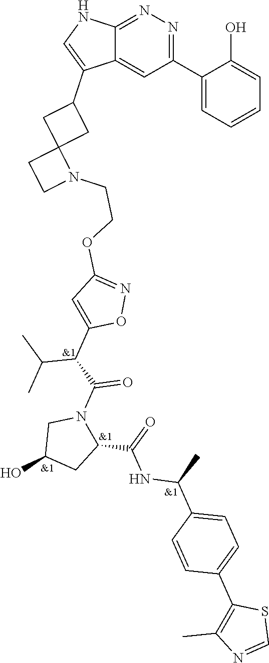 [SMILES: Cc1ncsc1-c1ccc([C@H](C)NC(=O)[C@@H]2C[C@@H](O)CN2C(=O)[C@@H](c2cc(OCCN3CCC34CC(c3c[nH]c5nnc(-c6ccccc6O)cc35)C4)no2)C(C)C)cc1] |
| 152 | 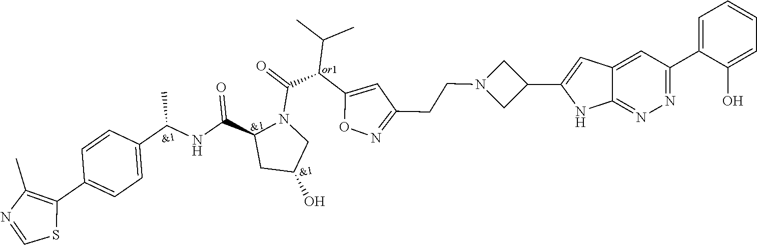 [SMILES: Cc1ncsc1-c1ccc([C@H](C)NC(=O)[C@@H]2C[C@@H](O)CN2C(=O)[C@@H](c2cc(CCN3CC(c4cc5cc(-c6ccccc6O)nnc5[nH]4)C3)no2)C(C)C)cc1] |
| 153 | 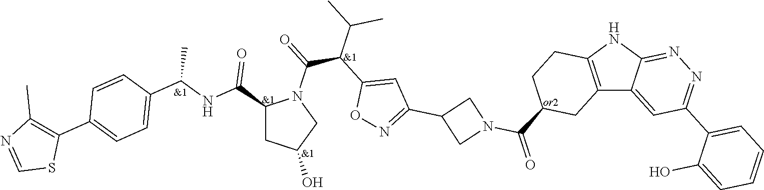 [SMILES: Cc1ncsc1-c1ccc([C@H](C)NC(=O)[C@@H]2C[C@@H](O)CN2C(=O)[C@H](c2cc(C3CN(C(=O)[C@H]4CCc5[nH]c6nnc(-c7ccccc7O)cc6c5C4)C3)no2)C(C)C)cc1] |
| 154 | 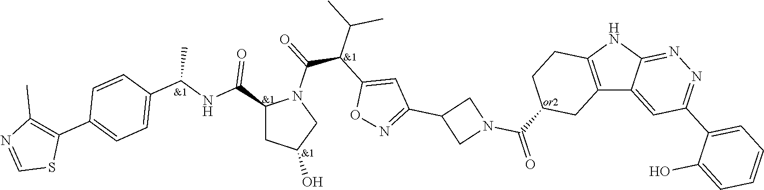 [SMILES: Cc1ncsc1-c1ccc([C@H](C)NC(=O)[C@@H]2C[C@@H](O)CN2C(=O)[C@H](c2cc(C3CN(C(=O)[C@@H]4CCc5[nH]c6nnc(-c7ccccc7O)cc6c5C4)C3)no2)C(C)C)cc1] |
| 155 | 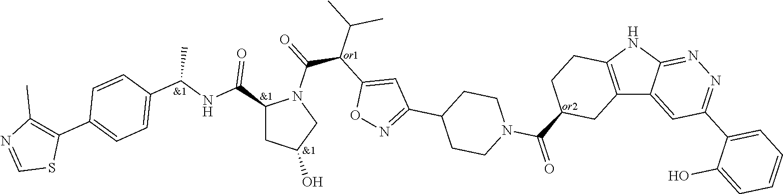 [SMILES: Cc1ncsc1-c1ccc([C@H](C)NC(=O)[C@@H]2C[C@@H](O)CN2C(=O)[C@H](c2cc(C3CCN(C(=O)[C@H]4CCc5[nH]c6nnc(-c7ccccc7O)cc6c5C4)CC3)no2)C(C)C)cc1] |
| 156 | 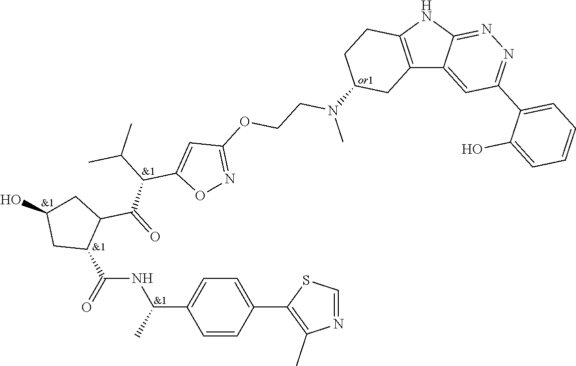 [SMILES: Cc1ncsc1-c1ccc([C@H](C)NC(=O)[C@@H]2C[C@@H](O)CC2C(=O)[C@@H](c2cc(OCCN(C)[C@@H]3CCc4[nH]c5nnc(-c6ccccc6O)cc5c4C3)no2)C(C)C)cc1] |
| 157 | 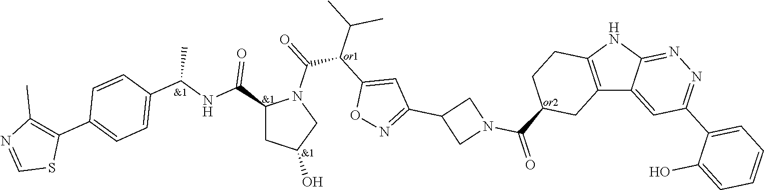 [SMILES: Cc1ncsc1-c1ccc([C@H](C)NC(=O)[C@@H]2C[C@@H](O)CN2C(=O)[C@@H](c2cc(C3CN(C(=O)[C@H]4CCc5[nH]c6nnc(-c7ccccc7O)cc6c5C4)C3)no2)C(C)C)cc1] |
| 158 | 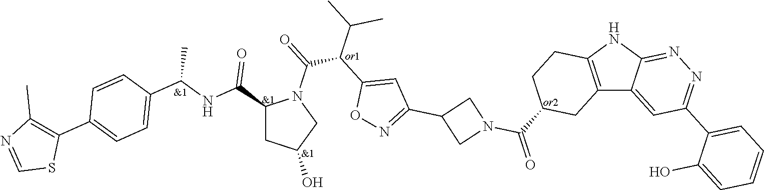 [SMILES: Cc1ncsc1-c1ccc([C@H](C)NC(=O)[C@@H]2C[C@@H](O)CN2C(=O)[C@@H](c2cc(C3CN(C(=O)[C@@H]4CCc5[nH]c6nnc(-c7ccccc7O)cc6c5C4)C3)no2)C(C)C)cc1] |
| 159 | 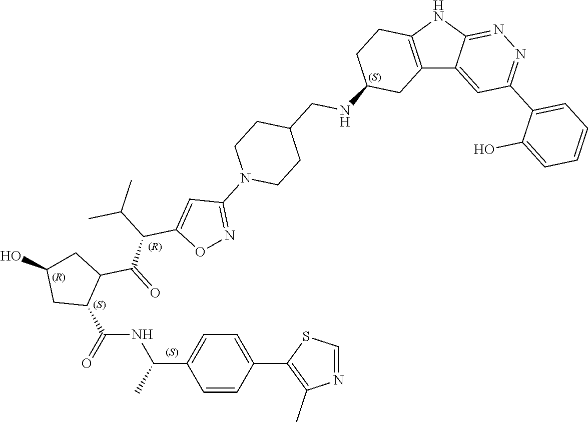 |
| 160 | 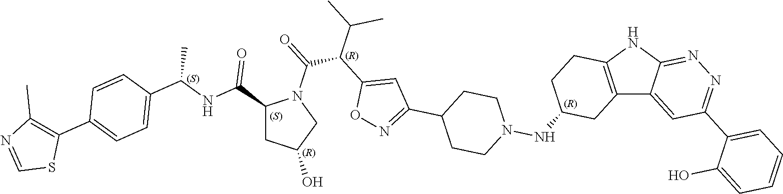 |
| 161 | 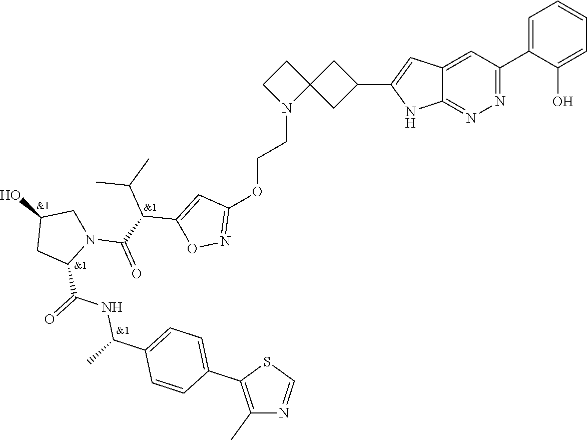 [SMILES: Cc1ncsc1-c1ccc([C@H](C)NC(=O)[C@@H]2C[C@@H](O)CN2C(=O)[C@@H](c2cc(OCCN3CCC34CC(c3cc5cc(-c6ccccc6O)nnc5[nH]3)C4)no2)C(C)C)cc1] |
| 162 | 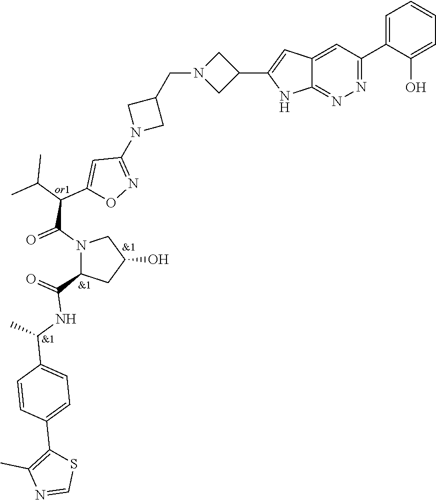 [SMILES: Cc1ncsc1-c1ccc([C@H](C)NC(=O)[C@@H]2C[C@@H](O)CN2C(=O)[C@H](c2cc(N3CC(CN4CC(c5cc6cc(-c7ccccc7O)nnc6[nH]5)C4)C3)no2)C(C)C)cc1] |
| 163 | 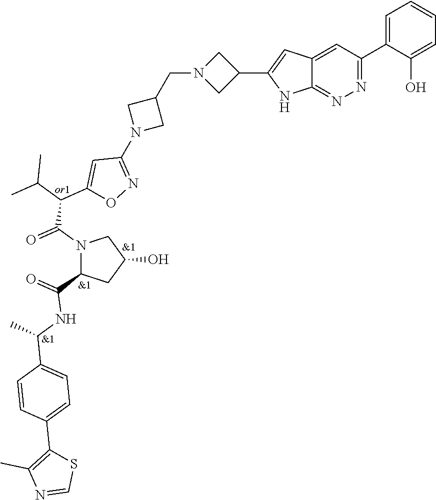 [SMILES: Cc1ncsc1-c1ccc([C@H](C)NC(=O)[C@@H]2C[C@@H](O)CN2C(=O)[C@@H](c2cc(N3CC(CN4CC(c5cc6cc(-c7ccccc7O)nnc6[nH]5)C4)C3)no2)C(C)C)cc1] |
| 164 | 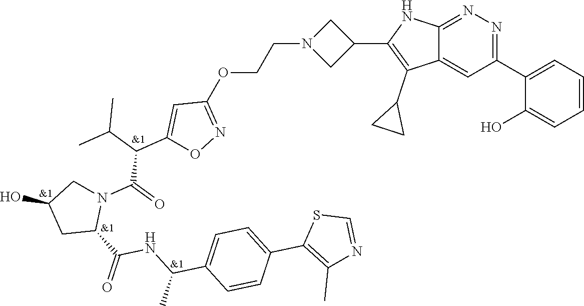 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6ccccc6O)cc5c4C4CC4)C3)no2)C(C)C)cc1] |
| 165 | 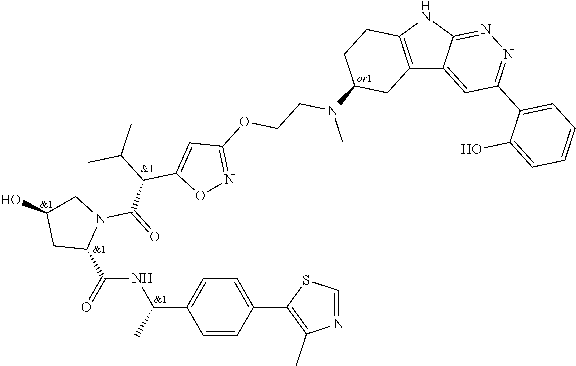 [SMILES: Cc1ncsc1-c1ccc([C@H](C)NC(=O)[C@@H]2C[C@@H](O)CN2C(=O)[C@@H](c2cc(OCCN(C)[C@H]3CCc4[nH]c5nnc(-c6ccccc6O)cc5c4C3)no2)C(C)C)cc1] |
| 166 | 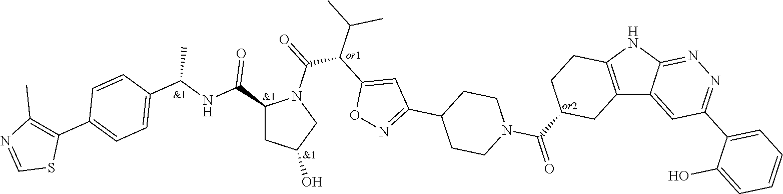 [SMILES: Cc1ncsc1-c1ccc([C@H](C)NC(=O)[C@@H]2C[C@@H](O)CN2C(=O)[C@@H](c2cc(C3CCN(C(=O)[C@@H]4CCc5[nH]c6nnc(-c7ccccc7O)cc6c5C4)CC3)no2)C(C)C)cc1] |
| 167 | 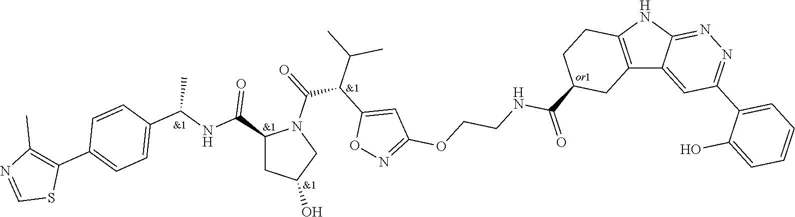 [SMILES: Cc1ncsc1-c1ccc([C@H](C)NC(=O)[C@@H]2C[C@@H](O)CN2C(=O)[C@@H](c2cc(OCCNC(=O)[C@H]3CCc4[nH]c5nnc(-c6ccccc6O)cc5c4C3)no2)C(C)C)cc1] |
| 168 | 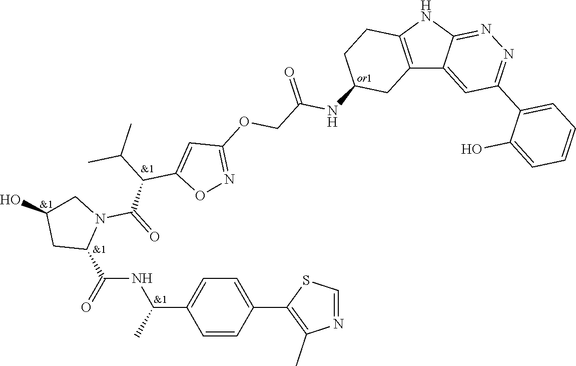 [SMILES: Cc1ncsc1-c1ccc([C@H](C)NC(=O)[C@@H]2C[C@@H](O)CN2C(=O)[C@@H](c2cc(OCC(=O)N[C@H]3CCc4[nH]c5nnc(-c6ccccc6O)cc5c4C3)no2)C(C)C)cc1] |
| 169 | 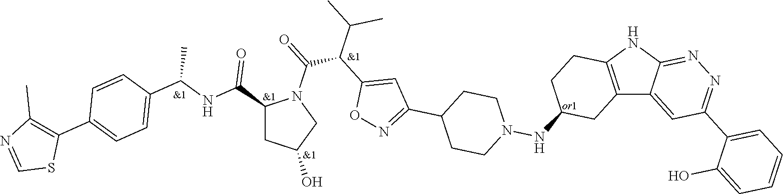 [SMILES: Cc1ncsc1-c1ccc([C@H](C)NC(=O)[C@@H]2C[C@@H](O)CN2C(=O)[C@@H](c2cc(C3CCN(N[C@H]4CCc5[nH]c6nnc(-c7ccccc7O)cc6c5C4)CC3)no2)C(C)C)cc1] |
| 170 | 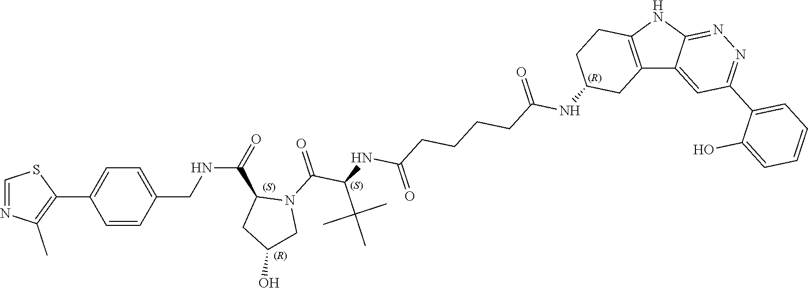 |
| 171 | 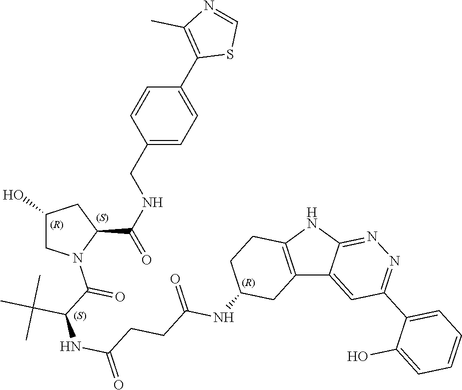 |
| 172 | 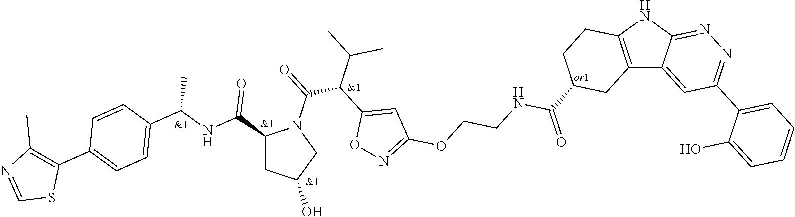 [SMILES: Cc1ncsc1-c1ccc([C@H](C)NC(=O)[C@@H]2C[C@@H](O)CN2C(=O)[C@@H](c2cc(OCCNC(=O)[C@@H]3CCc4[nH]c5nnc(-c6ccccc6O)cc5c4C3)no2)C(C)C)cc1] |
| 173 | 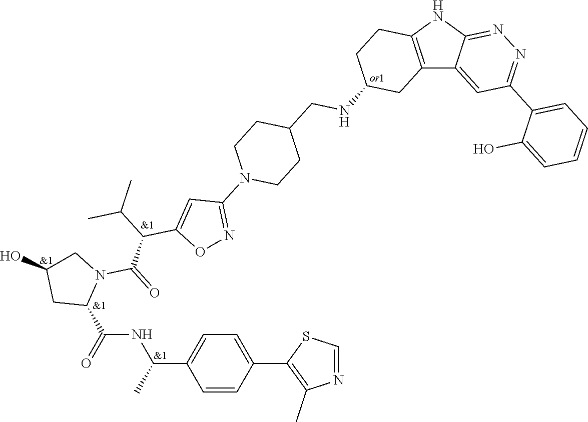 [SMILES: Cc1ncsc1-c1ccc([C@H](C)NC(=O)[C@@H]2C[C@@H](O)CN2C(=O)[C@@H](c2cc(N3CCC(CN[C@@H]4CCc5[nH]c6nnc(-c7ccccc7O)cc6c5C4)CC3)no2)C(C)C)cc1] |
| 174 | 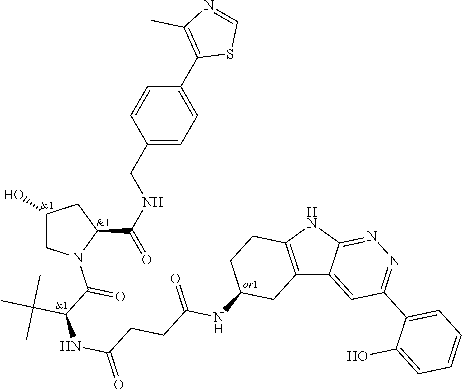 |
| 175 | 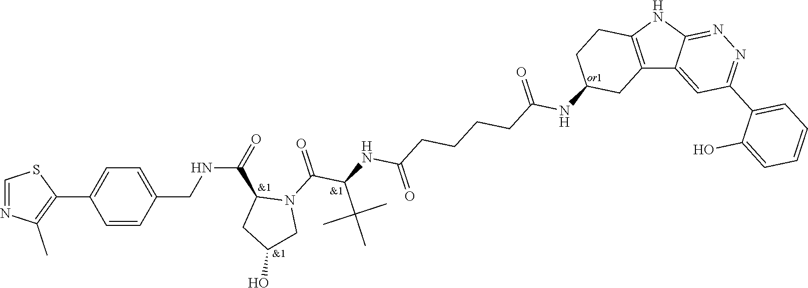 |
| 176 | 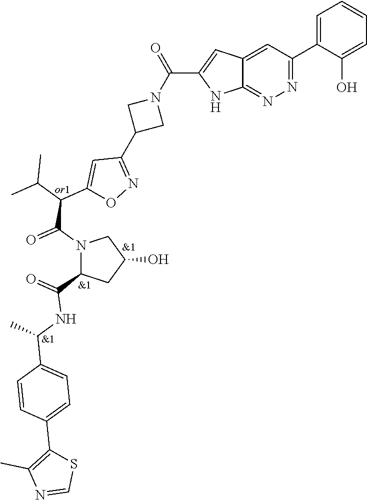 [SMILES: Cc1ncsc1-c1ccc([C@H](C)NC(=O)[C@@H]2C[C@@H](O)CN2C(=O)[C@H](c2cc(C3CN(C(=O)c4cc5cc(-c6ccccc6O)nnc5[nH]4)C3)no2)C(C)C)cc1] |
| 177 | 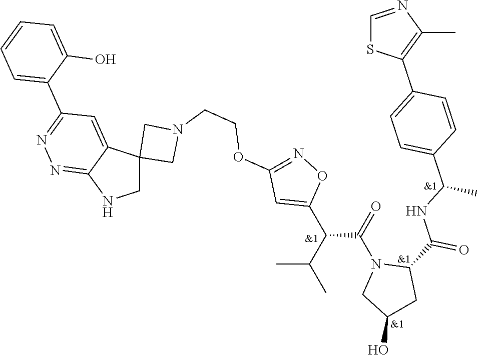 [SMILES: Cc1ncsc1-c1ccc([C@H](C)NC(=O)[C@@H]2C[C@@H](O)CN2C(=O)[C@@H](c2cc(OCCN3CC4(CNc5nnc(-c6ccccc6O)cc54)C3)no2)C(C)C)cc1] |
| 178 | 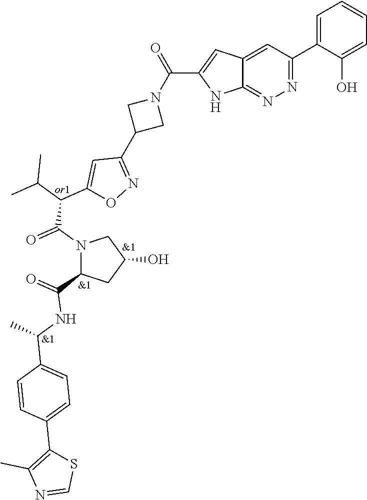 [SMILES: Cc1ncsc1-c1ccc([C@H](C)NC(=O)[C@@H]2C[C@@H](O)CN2C(=O)[C@@H](c2cc(C3CN(C(=O)c4cc5cc(-c6ccccc6O)nnc5[nH]4)C3)no2)C(C)C)cc1] |
| 179 | 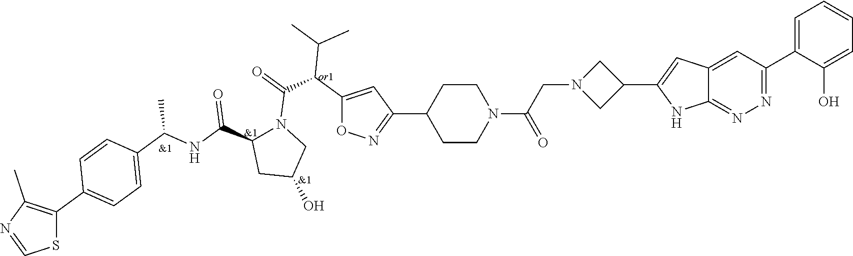 [SMILES: Cc1ncsc1-c1ccc([C@H](C)NC(=O)[C@@H]2C[C@@H](O)CN2C(=O)[C@@H](c2cc(C3CCN(C(=O)CN4CC(c5cc6cc(-c7ccccc7O)nnc6[nH]5)C4)CC3)no2)C(C)C)cc1] |
| 180 | 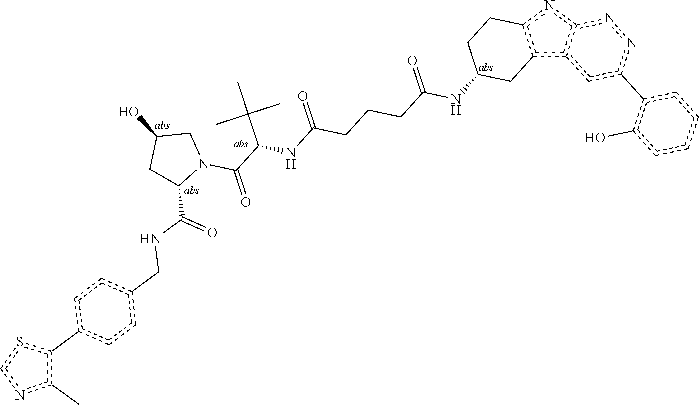 |
| 181 | 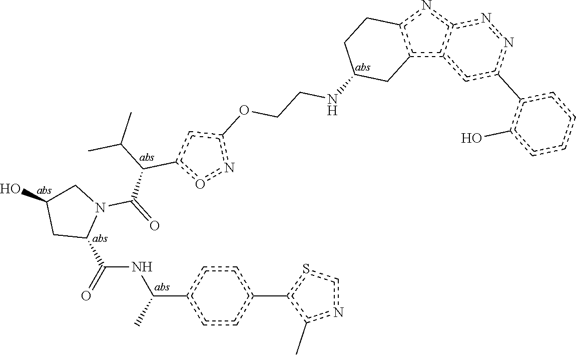 |
| 182 | 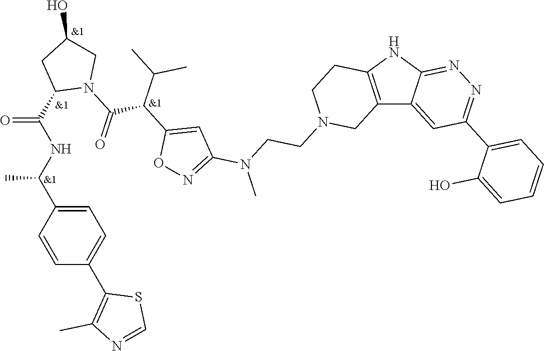 [SMILES: Cc1ncsc1-c1ccc([C@H](C)NC(=O)[C@@H]2C[C@@H](O)CN2C(=O)[C@@H](c2cc(N(C)CCN3CCc4[nH]c5nnc(-c6ccccc6O)cc5c4C3)no2)C(C)C)cc1] |
| 183 | 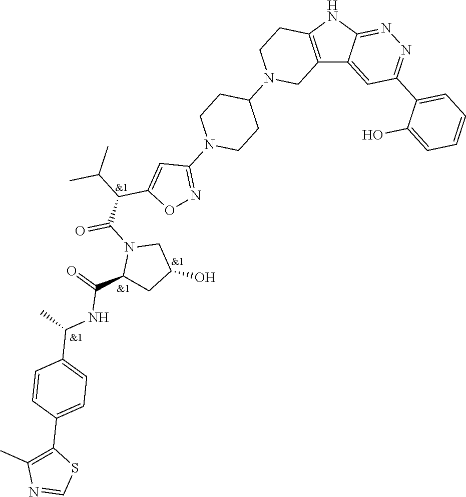 [SMILES: Cc1ncsc1-c1ccc([C@H](C)NC(=O)[C@@H]2C[C@@H](O)CN2C(=O)[C@@H](c2cc(N3CCC(N4CCc5[nH]c6nnc(-c7ccccc7O)cc6c5C4)CC3)no2)C(C)C)cc1] |
| 184 | 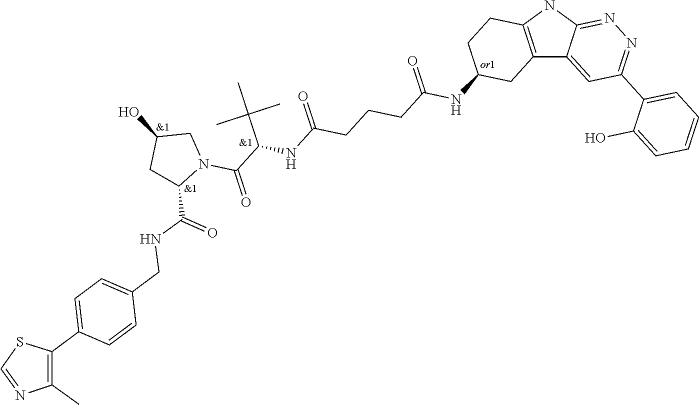 [SMILES: Cc1ncsc1-c1ccc(CNC(=O)[C@@H]2C[C@@H](O)CN2C(=O)[C@@H](NC(=O)CCCC(=O)N[C@H]2CCc3[nH]c4nnc(-c5ccccc5O)cc4c3C2)C(C)(C)C)cc1] |
| 185 | 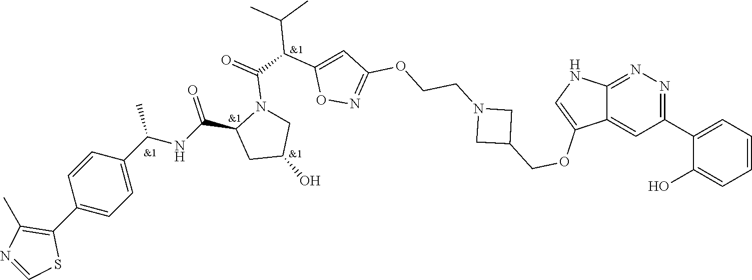 [SMILES: Cc1ncsc1-c1ccc([C@H](C)NC(=O)[C@@H]2C[C@@H](O)CN2C(=O)[C@@H](c2cc(OCCN3CC(COc4c[nH]c5nnc(-c6ccccc6O)cc45)C3)no2)C(C)C)cc1] |
| 186 | 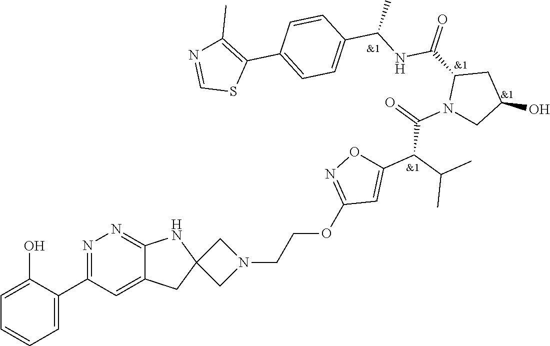 [SMILES: Cc1ncsc1-c1ccc([C@H](C)NC(=O)[C@@H]2C[C@@H](O)CN2C(=O)[C@@H](c2cc(OCCN3CC4(Cc5cc(-c6ccccc6O)nnc5N4)C3)no2)C(C)C)cc1] |
| 187 | 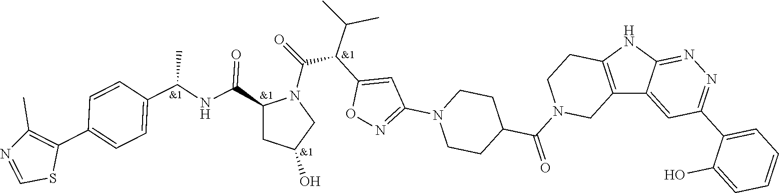 [SMILES: Cc1ncsc1-c1ccc([C@H](C)NC(=O)[C@@H]2C[C@@H](O)CN2C(=O)[C@@H](c2cc(N3CCC(C(=O)N4CCc5[nH]c6nnc(-c7ccccc7O)cc6c5C4)CC3)no2)C(C)C)cc1] |
| 188 | 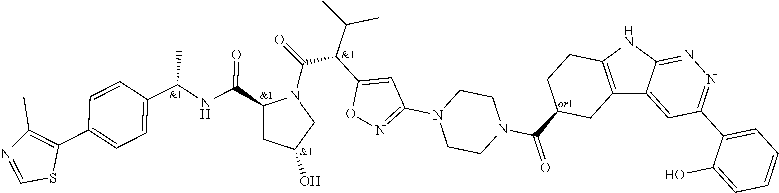 [SMILES: Cc1ncsc1-c1ccc([C@H](C)NC(=O)[C@@H]2C[C@@H](O)CN2C(=O)[C@@H](c2cc(N3CCN(C(=O)[C@H]4CCc5[nH]c6nnc(-c7ccccc7O)cc6c5C4)CC3)no2)C(C)C)cc1] |
| 189 | 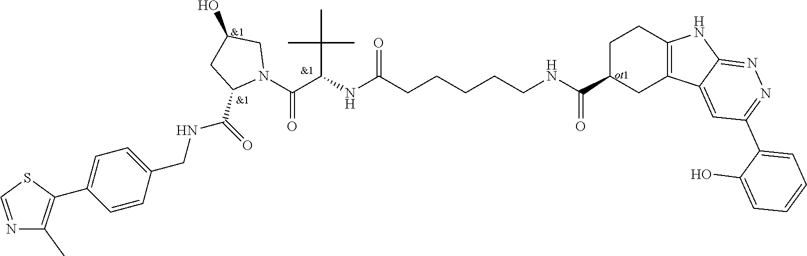 [SMILES: Cc1ncsc1-c1ccc(CNC(=O)[C@@H]2C[C@@H](O)CN2C(=O)[C@@H](NC(=O)CCCCCNC(=O)[C@H]2CCc3[nH]c4nnc(-c5ccccc5O)cc4c3C2)C(C)(C)C)cc1] |
| 190 | 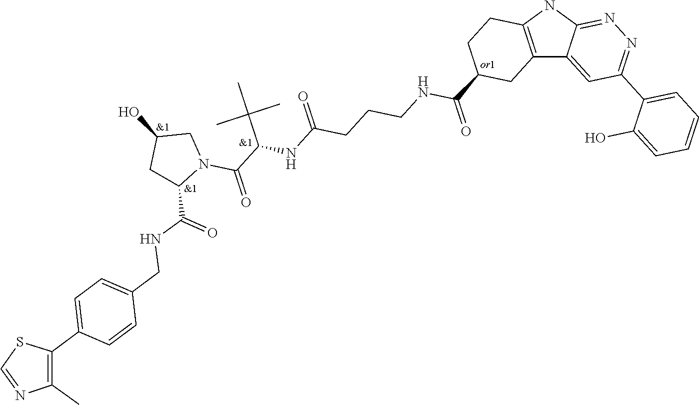 [SMILES: Cc1ncsc1-c1ccc(CNC(=O)[C@@H]2C[C@@H](O)CN2C(=O)[C@@H](NC(=O)CCCNC(=O)[C@H]2CCc3[nH]c4nnc(-c5ccccc5O)cc4c3C2)C(C)(C)C)cc1] |
| 191 | 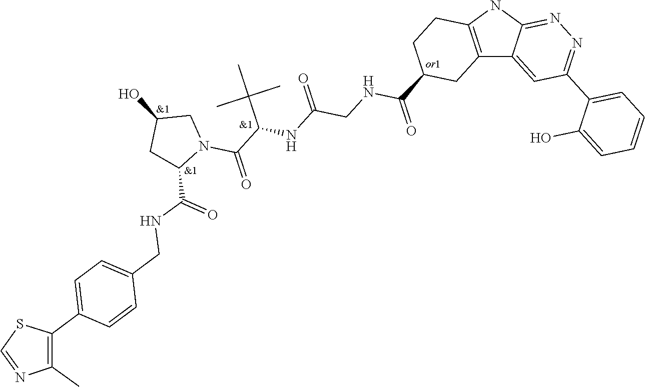 [SMILES: Cc1ncsc1-c1ccc(CNC(=O)[C@@H]2C[C@@H](O)CN2C(=O)[C@@H](NC(=O)CNC(=O)[C@H]2CCc3[nH]c4nnc(-c5ccccc5O)cc4c3C2)C(C)(C)C)cc1] |
| 192 | 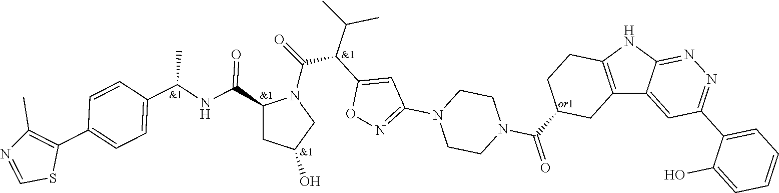 [SMILES: Cc1ncsc1-c1ccc([C@H](C)NC(=O)[C@@H]2C[C@@H](O)CN2C(=O)[C@@H](c2cc(N3CCN(C(=O)[C@@H]4CCc5[nH]c6nnc(-c7ccccc7O)cc6c5C4)CC3)no2)C(C)C)cc1] |
| 193 | 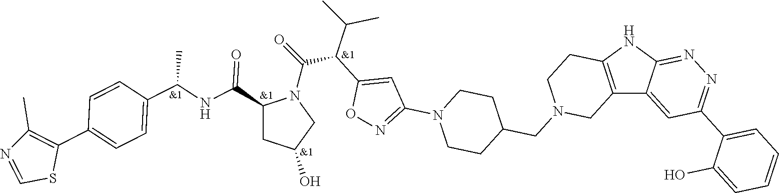 [SMILES: Cc1ncsc1-c1ccc([C@H](C)NC(=O)[C@@H]2C[C@@H](O)CN2C(=O)[C@@H](c2cc(N3CCC(CN4CCc5[nH]c6nnc(-c7ccccc7O)cc6c5C4)CC3)no2)C(C)C)cc1] |
| 194 | 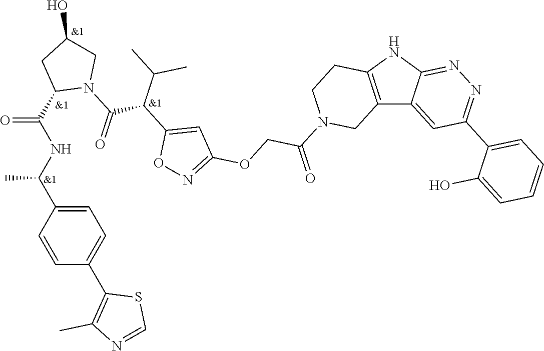 [SMILES: Cc1ncsc1-c1ccc([C@H](C)NC(=O)[C@@H]2C[C@@H](O)CN2C(=O)[C@@H](c2cc(OCC(=O)N3CCc4[nH]c5nnc(-c6ccccc6O)cc5c4C3)no2)C(C)C)cc1] |
| 195 | 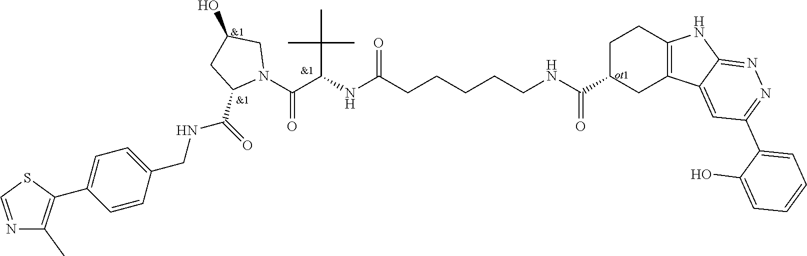 [SMILES: Cc1ncsc1-c1ccc(CNC(=O)[C@@H]2C[C@@H](O)CN2C(=O)[C@@H](NC(=O)CCCCCNC(=O)[C@@H]2CCc3[nH]c4nnc(-c5ccccc5O)cc4c3C2)C(C)(C)C)cc1] |
| 196 | 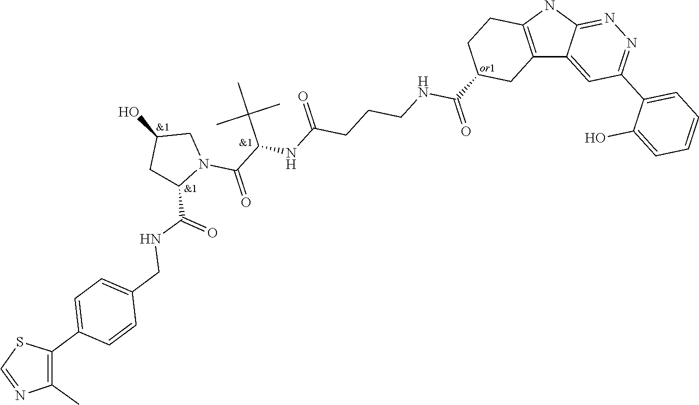 [SMILES: Cc1ncsc1-c1ccc(CNC(=O)[C@@H]2C[C@@H](O)CN2C(=O)[C@@H](NC(=O)CCCNC(=O)[C@@H]2CCc3[nH]c4nnc(-c5ccccc5O)cc4c3C2)C(C)(C)C)cc1] |
| 197 | 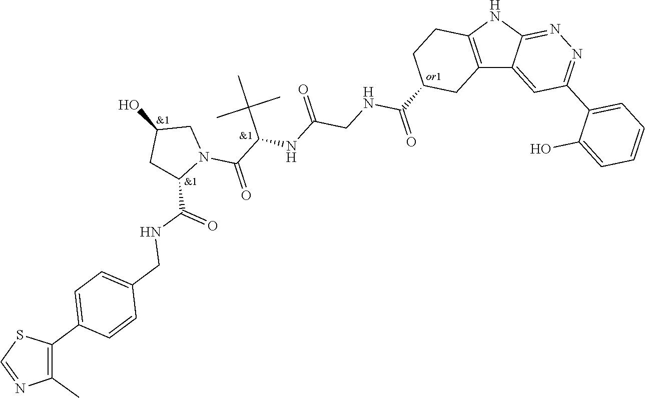 [SMILES: Cc1ncsc1-c1ccc(CNC(=O)[C@@H]2C[C@@H](O)CN2C(=O)[C@@H](NC(=O)CNC(=O)[C@@H]2CCc3[nH]c4nnc(-c5ccccc5O)cc4c3C2)C(C)(C)C)cc1] |
| 198 | 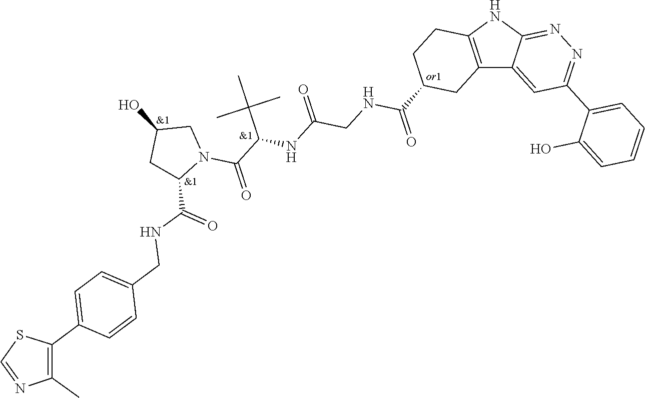 [SMILES: Cc1ncsc1-c1ccc(CNC(=O)[C@@H]2C[C@@H](O)CN2C(=O)[C@@H](NC(=O)CNC(=O)[C@@H]2CCc3[nH]c4nnc(-c5ccccc5O)cc4c3C2)C(C)(C)C)cc1] |
| 199 | 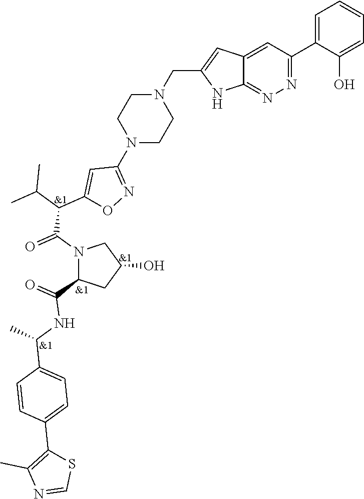 [SMILES: Cc1ncsc1-c1ccc([C@H](C)NC(=O)[C@@H]2C[C@@H](O)CN2C(=O)[C@@H](c2cc(N3CCN(Cc4cc5cc(-c6ccccc6O)nnc5[nH]4)CC3)no2)C(C)C)cc1] |
| 200 | 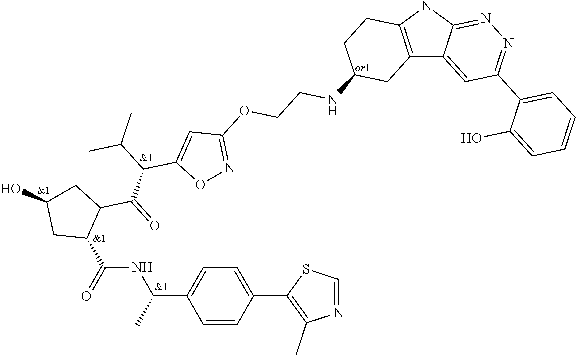 [SMILES: Cc1ncsc1-c1ccc([C@H](C)NC(=O)[C@@H]2C[C@@H](O)CC2C(=O)[C@@H](c2cc(OCCN[C@H]3CCc4[nH]c5nnc(-c6ccccc6O)cc5c4C3)no2)C(C)C)cc1] |
| 201 | 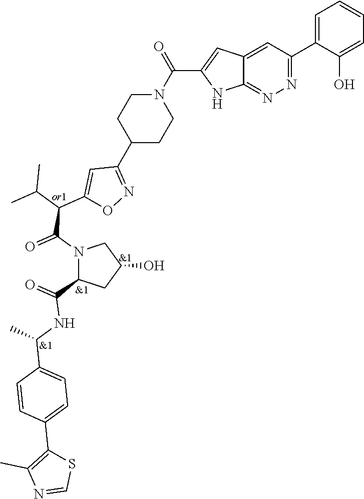 [SMILES: Cc1ncsc1-c1ccc([C@H](C)NC(=O)[C@@H]2C[C@@H](O)CN2C(=O)[C@H](c2cc(C3CCN(C(=O)c4cc5cc(-c6ccccc6O)nnc5[nH]4)CC3)no2)C(C)C)cc1] |
| 202 | 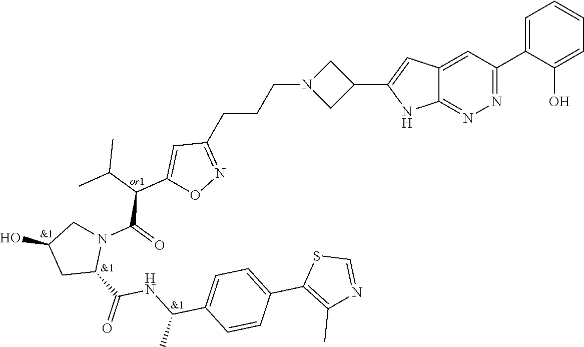 [SMILES: Cc1ncsc1-c1ccc([C@H](C)NC(=O)[C@@H]2C[C@@H](O)CN2C(=O)[C@H](c2cc(CCCN3CC(c4cc5cc(-c6ccccc6O)nnc5[nH]4)C3)no2)C(C)C)cc1] |
| 203 | 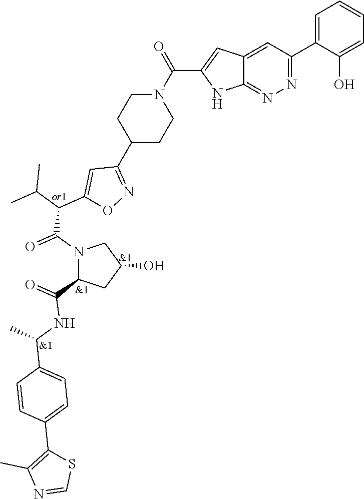 [SMILES: Cc1ncsc1-c1ccc([C@H](C)NC(=O)[C@@H]2C[C@@H](O)CN2C(=O)[C@@H](c2cc(C3CCN(C(=O)c4cc5cc(-c6ccccc6O)nnc5[nH]4)CC3)no2)C(C)C)cc1] |
| 204 | 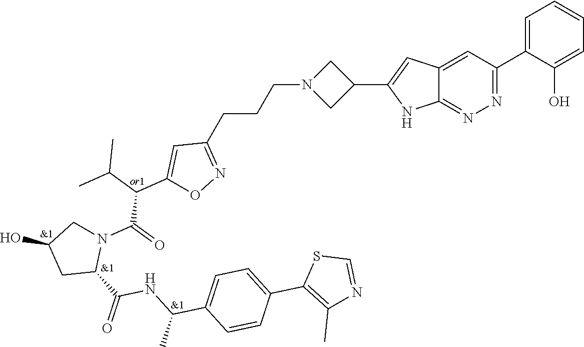 [SMILES: Cc1ncsc1-c1ccc([C@H](C)NC(=O)[C@@H]2C[C@@H](O)CN2C(=O)[C@@H](c2cc(CCCN3CC(c4cc5cc(-c6ccccc6O)nnc5[nH]4)C3)no2)C(C)C)cc1] |
| 205 | 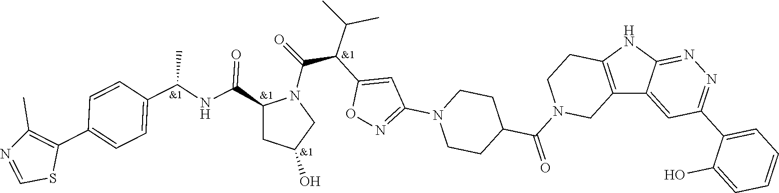 [SMILES: Cc1ncsc1-c1ccc([C@H](C)NC(=O)[C@@H]2C[C@@H](O)CN2C(=O)[C@H](c2cc(N3CCC(C(=O)N4CCc5[nH]c6nnc(-c7ccccc7O)cc6c5C4)CC3)no2)C(C)C)cc1] |
| 206 | 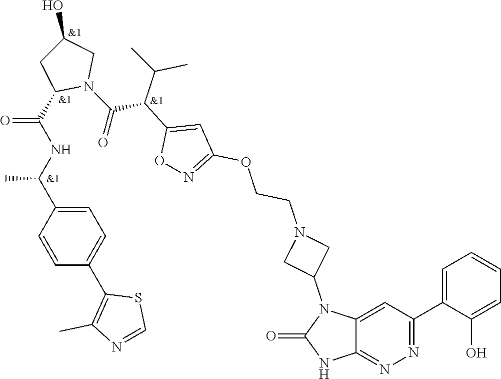 [SMILES: Cc1ncsc1-c1ccc([C@H](C)NC(=O)[C@@H]2C[C@@H](O)CN2C(=O)[C@@H](c2cc(OCCN3CC(n4c(=O)[nH]c5nnc(-c6ccccc6O)cc54)C3)no2)C(C)C)cc1] |
| 207 | 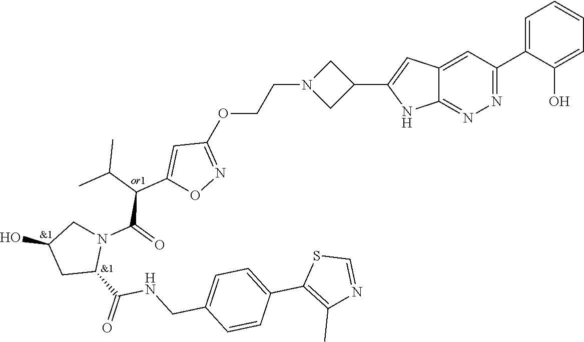 [SMILES: Cc1ncsc1-c1ccc(CNC(=O)[C@@H]2C[C@@H](O)CN2C(=O)[C@H](c2cc(OCCN3CC(c4cc5cc(-c6ccccc6O)nnc5[nH]4)C3)no2)C(C)C)cc1] |
| 208 | 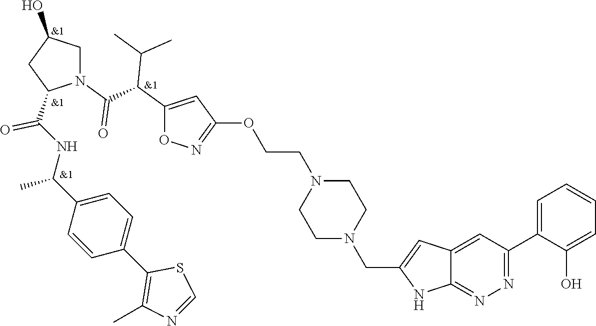 [SMILES: Cc1ncsc1-c1ccc([C@H](C)NC(=O)[C@@H]2C[C@@H](O)CN2C(=O)[C@@H](c2cc(OCCN3CCN(Cc4cc5cc(-c6ccccc6O)nnc5[nH]4)CC3)no2)C(C)C)cc1] |
| 209 | 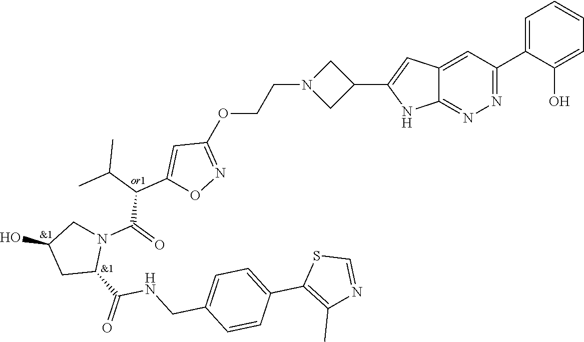 [SMILES: Cc1ncsc1-c1ccc(CNC(=O)[C@@H]2C[C@@H](O)CN2C(=O)[C@@H](c2cc(OCCN3CC(c4cc5cc(-c6ccccc6O)nnc5[nH]4)C3)no2)C(C)C)cc1] |
| 210 | 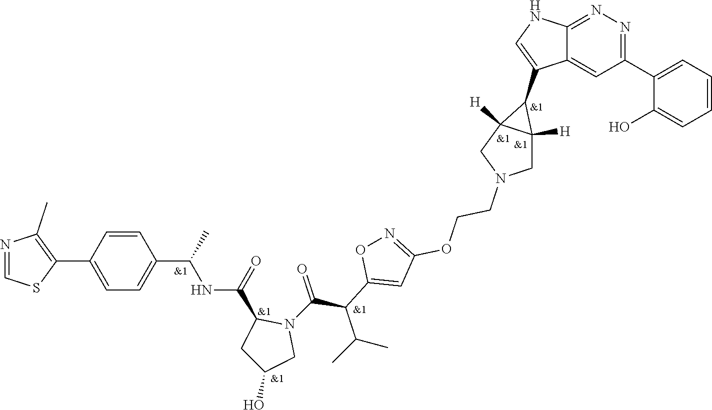 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3c[nH]c4nnc(-c5ccccc5O)cc34)no2)C(C)C)cc1] |
| 211 | 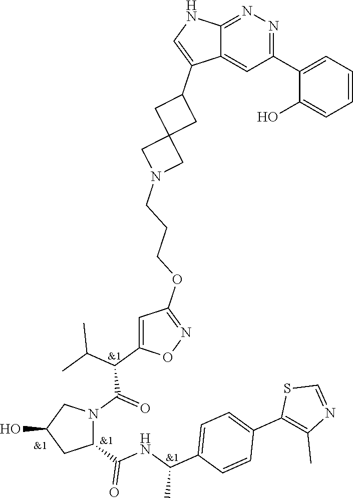 [SMILES: Cc1ncsc1-c1ccc([C@H](C)NC(=O)[C@@H]2C[C@@H](O)CN2C(=O)[C@@H](c2cc(OCCCN3CC4(CC(c5c[nH]c6nnc(-c7ccccc7O)cc56)C4)C3)no2)C(C)C)cc1] |
| 212 | 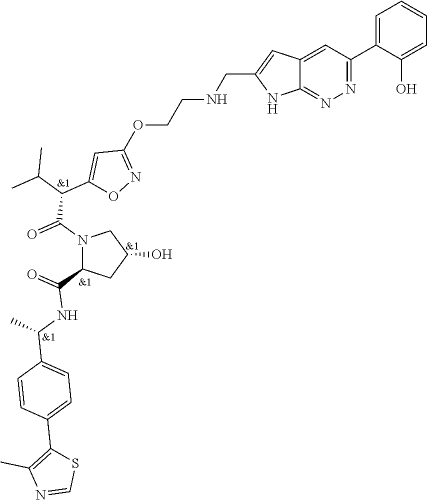 [SMILES: Cc1ncsc1-c1ccc([C@H](C)NC(=O)[C@@H]2C[C@@H](O)CN2C(=O)[C@@H](c2cc(OCCNCc3cc4cc(-c5ccccc5O)nnc4[nH]3)no2)C(C)C)cc1] |
| 213 | 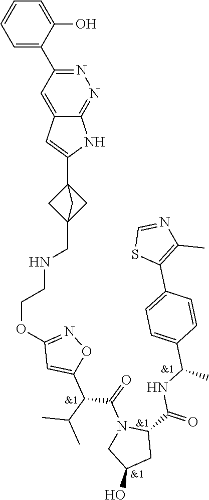 [SMILES: Cc1ncsc1-c1ccc([C@H](C)NC(=O)[C@@H]2C[C@@H](O)CN2C(=O)[C@@H](c2cc(OCCNCC34CC(c5cc6cc(-c7ccccc7O)nnc6[nH]5)(C3)C4)no2)C(C)C)cc1] |
| 214 | 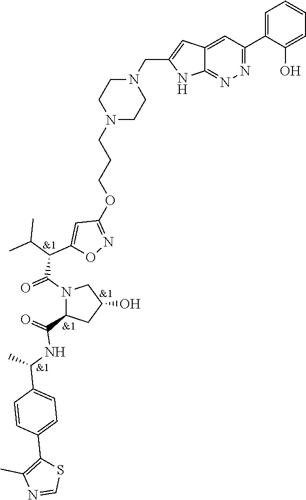 [SMILES: Cc1ncsc1-c1ccc([C@H](C)NC(=O)[C@@H]2C[C@@H](O)CN2C(=O)[C@@H](c2cc(OCCCN3CCN(Cc4cc5cc(-c6ccccc6O)nnc5[nH]4)CC3)no2)C(C)C)cc1] |
| 215 | 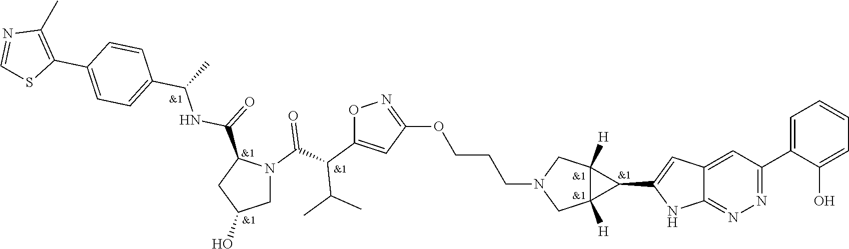 [SMILES: Cc1ncsc1-c1ccc([C@H](C)NC(=O)[C@@H]2C[C@@H](O)CN2C(=O)[C@H](c2cc(OCCCN3C[C@@H]4[C@H](C3)[C@H]4c3cc4cc(-c5ccccc5O)nnc4[nH]3)no2)C(C)C)cc1] |
| 216 | 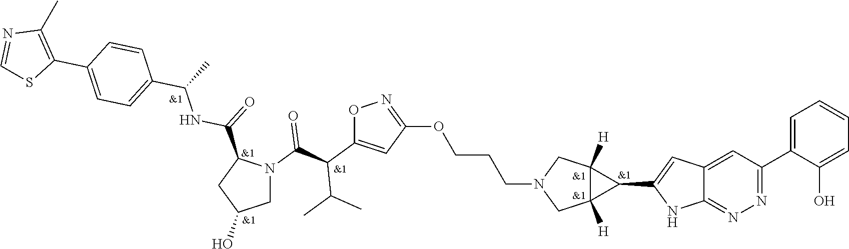 [SMILES: Cc1ncsc1-c1ccc([C@H](C)NC(=O)[C@@H]2C[C@@H](O)CN2C(=O)[C@@H](c2cc(OCCCN3C[C@@H]4[C@H](C3)[C@H]4c3cc4cc(-c5ccccc5O)nnc4[nH]3)no2)C(C)C)cc1] |
| 217 | 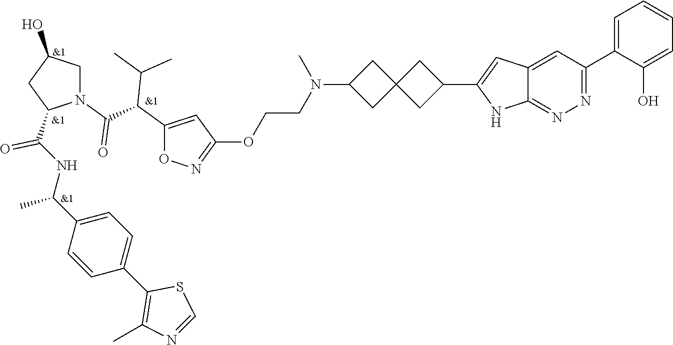 [SMILES: Cc1ncsc1-c1ccc([C@H](C)NC(=O)[C@@H]2C[C@@H](O)CN2C(=O)[C@@H](c2cc(OCCN(C)C3CC4(CC(c5cc6cc(-c7ccccc7O)nnc6[nH]5)C4)C3)no2)C(C)C)cc1] |
| 218 | 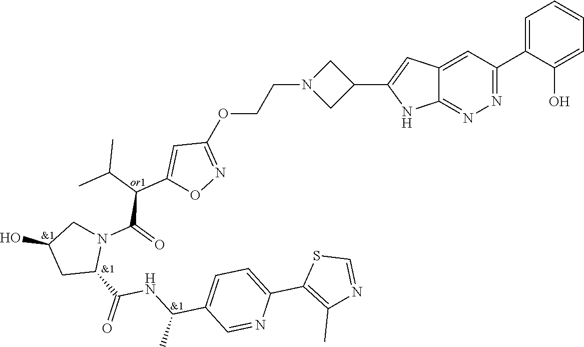 [SMILES: Cc1ncsc1-c1ccc([C@H](C)NC(=O)[C@@H]2C[C@@H](O)CN2C(=O)[C@H](c2cc(OCCN3CC(c4cc5cc(-c6ccccc6O)nnc5[nH]4)C3)no2)C(C)C)cn1] |
| 219 | 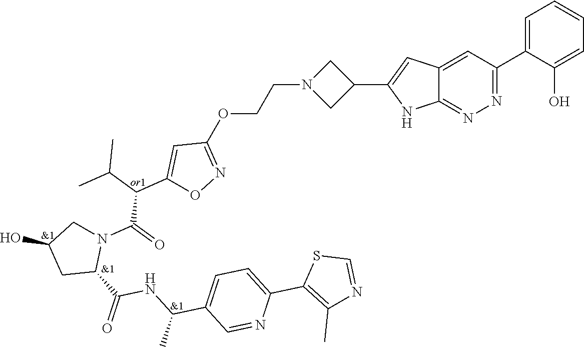 [SMILES: Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5[nH]4)C3)no2)C(C)C)cn1] |
| 220 | 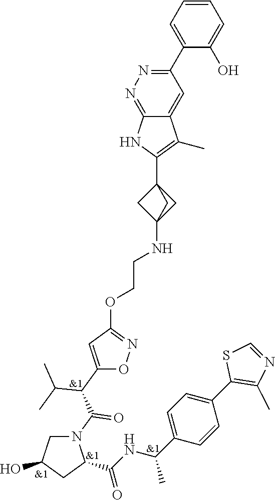 [SMILES: Cc1ncsc1-c1ccc([C@H](C)NC(=O)[C@@H]2C[C@@H](O)CN2C(=O)[C@@H](c2cc(OCCNC34CC(c5[nH]c6nnc(-c7ccccc7O)cc6c5C)(C3)C4)no2)C(C)C)cc1] |
| 221 | 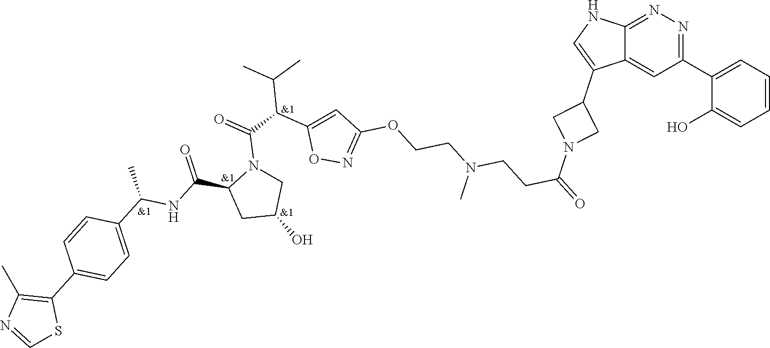 [SMILES: Cc1ncsc1-c1ccc([C@H](C)NC(=O)[C@@H]2C[C@@H](O)CN2C(=O)[C@@H](c2cc(OCCN(C)CCC(=O)N3CC(c4c[nH]c5nnc(-c6ccccc6O)cc45)C3)no2)C(C)C)cc1] |
| 222 | 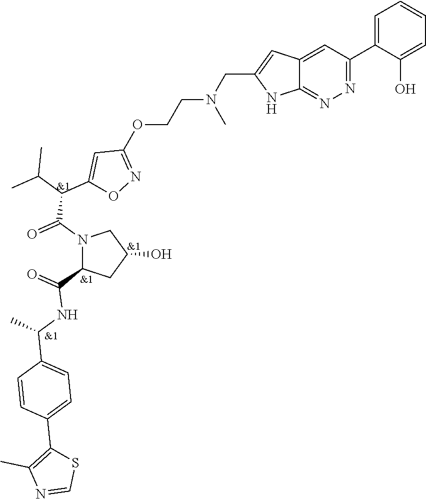 [SMILES: Cc1ncsc1-c1ccc([C@H](C)NC(=O)[C@@H]2C[C@@H](O)CN2C(=O)[C@@H](c2cc(OCCN(C)Cc3cc4cc(-c5ccccc5O)nnc4[nH]3)no2)C(C)C)cc1] |
| 223 | 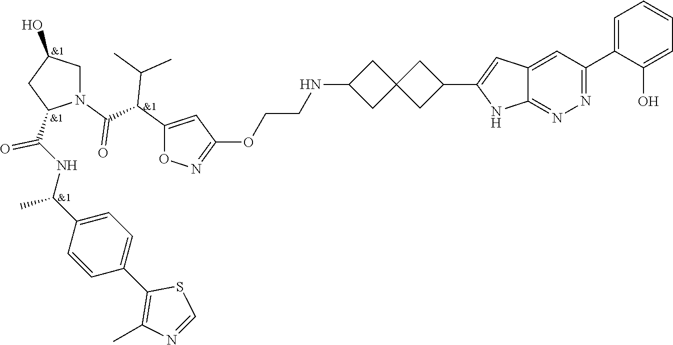 [SMILES: Cc1ncsc1-c1ccc([C@H](C)NC(=O)[C@@H]2C[C@@H](O)CN2C(=O)[C@@H](c2cc(OCCNC3CC4(C3)CC(c3cc5cc(-c6ccccc6O)nnc5[nH]3)C4)no2)C(C)C)cc1] |
| 224 | 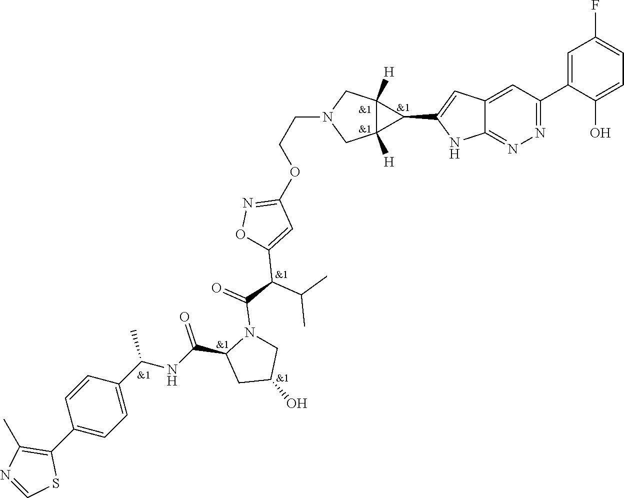 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3cc4cc(-c5cc(F)ccc5O)nnc4[nH]3)no2)C(C)C)cc1] |
| 225 | 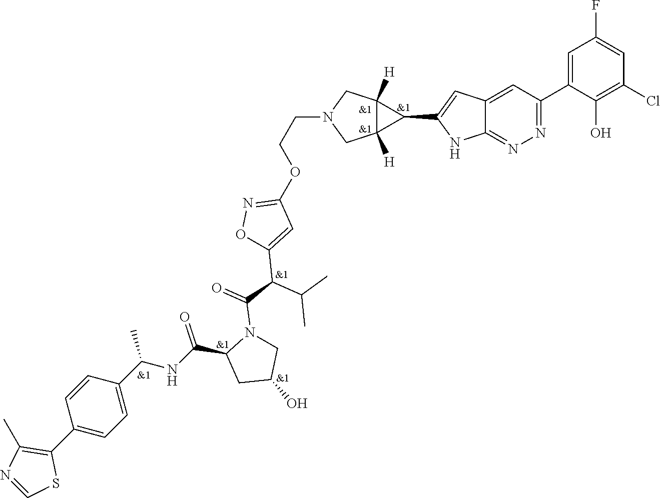 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3cc4cc(-c5cc(F)cc(Cl)c5O)nnc4[nH]3)no2)C(C)C)cc1] |
| 226 | 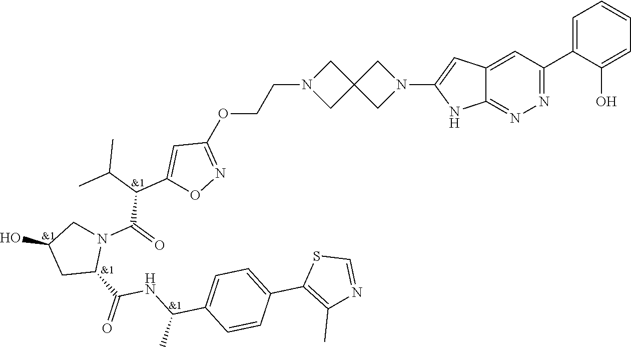 [SMILES: Cc1ncsc1-c1ccc([C@H](C)NC(=O)[C@@H]2C[C@@H](O)CN2C(=O)[C@@H](c2cc(OCCN3CC4(C3)CN(c3cc5cc(-c6ccccc6O)nnc5[nH]3)C4)no2)C(C)C)cc1] |
| 227 | 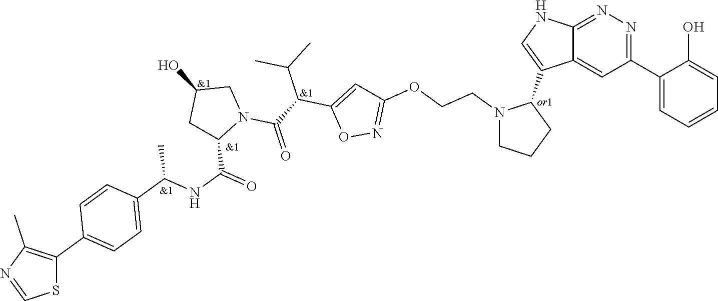 [SMILES: Cc1ncsc1-c1ccc([C@H](C)NC(=O)[C@@H]2C[C@@H](O)CN2C(=O)[C@@H](c2cc(OCCN3CCC[C@H]3c3c[nH]c4nnc(-c5ccccc5O)cc34)no2)C(C)C)cc1] |
| 228 | 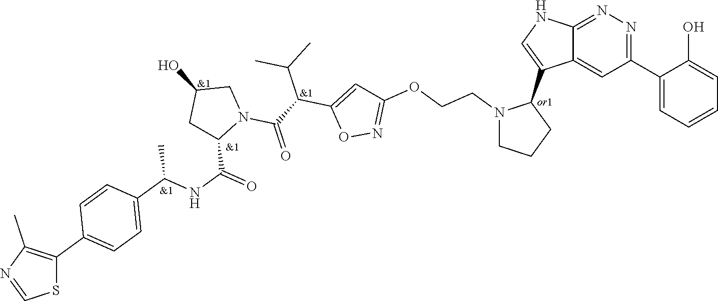 [SMILES: Cc1ncsc1-c1ccc([C@H](C)NC(=O)[C@@H]2C[C@@H](O)CN2C(=O)[C@@H](c2cc(OCCN3CCC[C@@H]3c3c[nH]c4nnc(-c5ccccc5O)cc34)no2)C(C)C)cc1] |
| 229 | 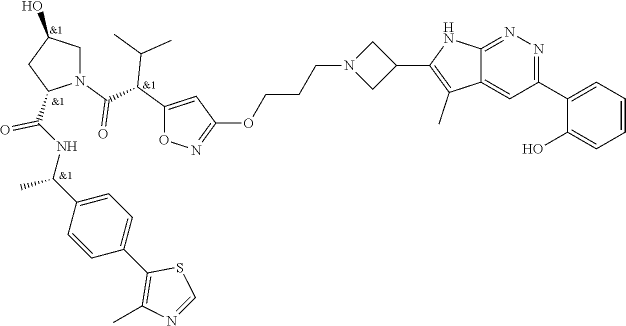 [SMILES: Cc1ncsc1-c1ccc([C@H](C)NC(=O)[C@@H]2C[C@@H](O)CN2C(=O)[C@@H](c2cc(OCCCN3CC(c4[nH]c5nnc(-c6ccccc6O)cc5c4C)C3)no2)C(C)C)cc1] |
| 230 | 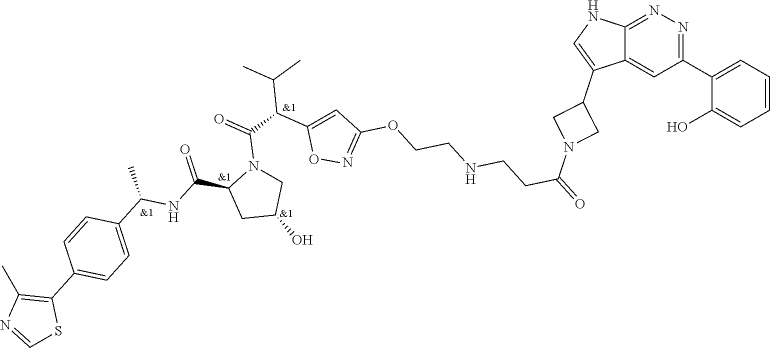 [SMILES: Cc1ncsc1-c1ccc([C@H](C)NC(=O)[C@@H]2C[C@@H](O)CN2C(=O)[C@@H](c2cc(OCCNCCC(=O)N3CC(c4c[nH]c5nnc(-c6ccccc6O)cc45)C3)no2)C(C)C)cc1] |
| 231 | 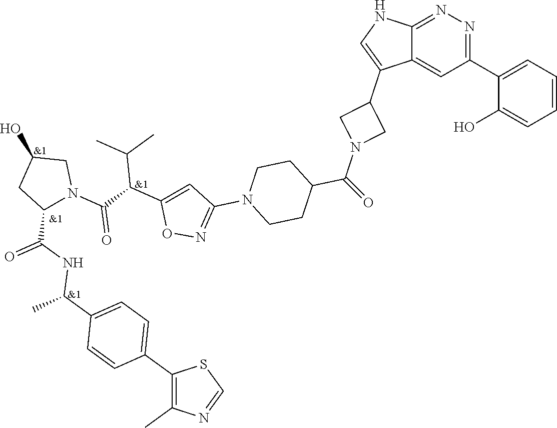 [SMILES: Cc1ncsc1-c1ccc([C@H](C)NC(=O)[C@@H]2C[C@@H](O)CN2C(=O)[C@@H](c2cc(N3CCC(C(=O)N4CC(c5c[nH]c6nnc(-c7ccccc7O)cc56)C4)CC3)no2)C(C)C)cc1] |
| 232 | 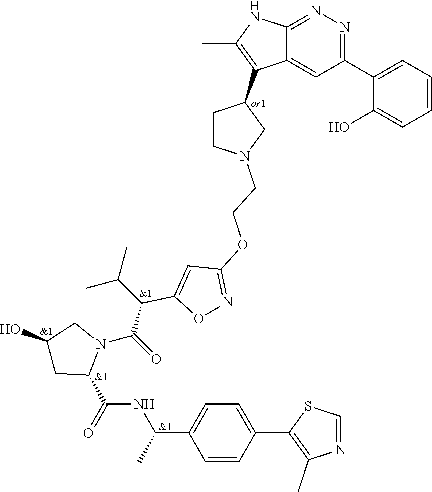 [SMILES: Cc1ncsc1-c1ccc([C@H](C)NC(=O)[C@@H]2C[C@@H](O)CN2C(=O)[C@@H](c2cc(OCCN3CC[C@@H](c4c(C)[nH]c5nnc(-c6ccccc6O)cc45)C3)no2)C(C)C)cc1] |
| 233 | 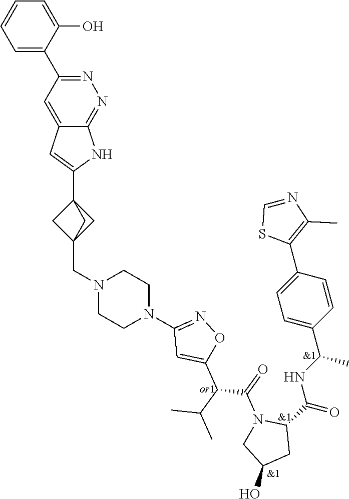 [SMILES: Cc1ncsc1-c1ccc([C@H](C)NC(=O)[C@@H]2C[C@@H](O)CN2C(=O)[C@@H](c2cc(N3CCN(CC45CC(c6cc7cc(-c8ccccc8O)nnc7[nH]6)(C4)C5)CC3)no2)C(C)C)cc1] |
| 234 | 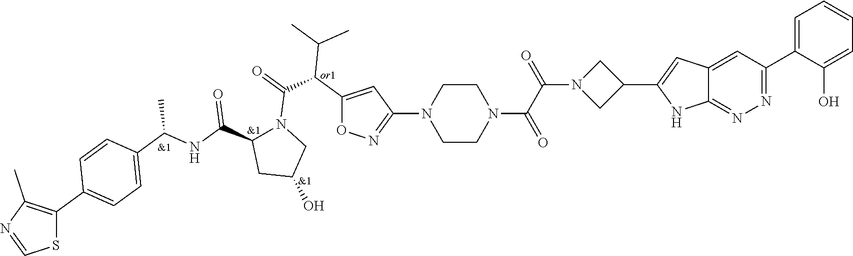 [SMILES: Cc1ncsc1-c1ccc([C@H](C)NC(=O)[C@@H]2C[C@@H](O)CN2C(=O)[C@@H](c2cc(N3CCN(C(=O)C(=O)N4CC(c5cc6cc(-c7ccccc7O)nnc6[nH]5)C4)CC3)no2)C(C)C)cc1] |
| 235 | 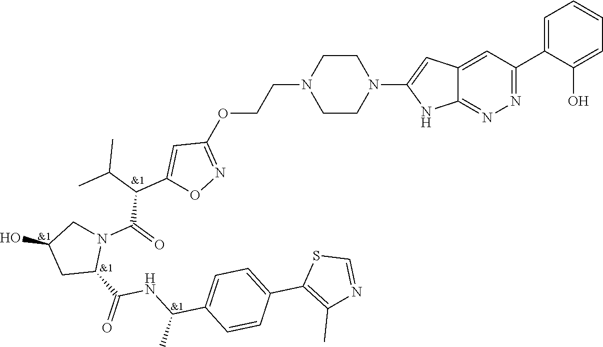 [SMILES: Cc1ncsc1-c1ccc([C@H](C)NC(=O)[C@@H]2C[C@@H](O)CN2C(=O)[C@@H](c2cc(OCCN3CCN(c4cc5cc(-c6ccccc6O)nnc5[nH]4)CC3)no2)C(C)C)cc1] |
| 236 | 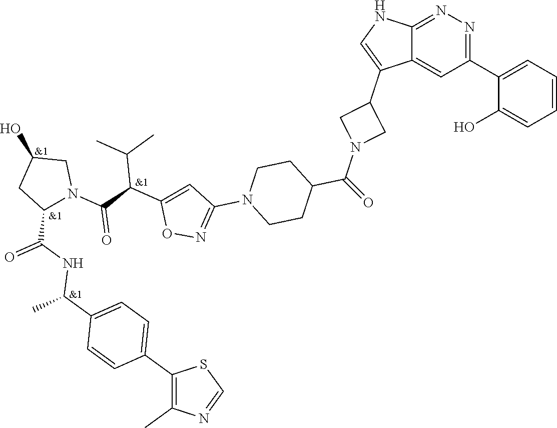 [SMILES: Cc1ncsc1-c1ccc([C@H](C)NC(=O)[C@@H]2C[C@@H](O)CN2C(=O)[C@H](c2cc(N3CCC(C(=O)N4CC(c5c[nH]c6nnc(-c7ccccc7O)cc56)C4)CC3)no2)C(C)C)cc1] |
| 237 | 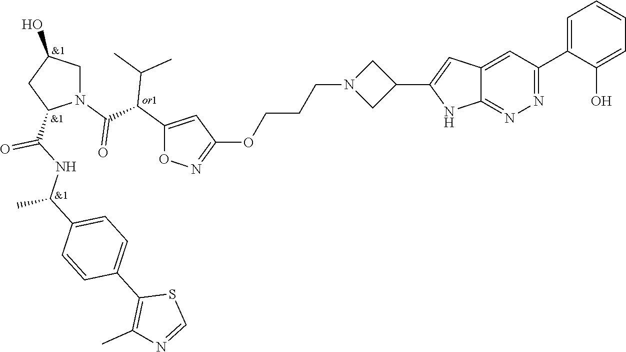 [SMILES: Cc1ncsc1-c1ccc([C@H](C)NC(=O)[C@@H]2C[C@@H](O)CN2C(=O)[C@@H](c2cc(OCCCN3CC(c4cc5cc(-c6ccccc6O)nnc5[nH]4)C3)no2)C(C)C)cc1] |
| 238 | 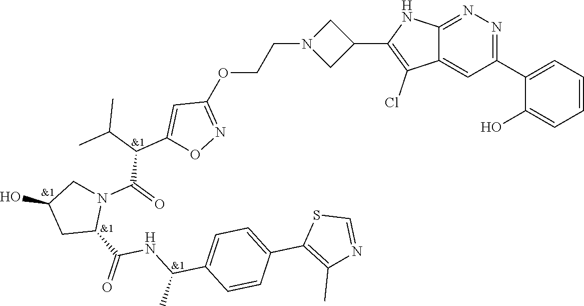 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6ccccc6O)cc5c4Cl)C3)no2)C(C)C)cc1] |
| 239 | 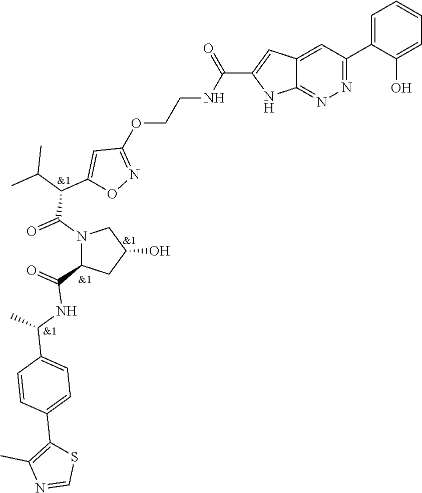 [SMILES: Cc1ncsc1-c1ccc([C@H](C)NC(=O)[C@@H]2C[C@@H](O)CN2C(=O)[C@@H](c2cc(OCCNC(=O)c3cc4cc(-c5ccccc5O)nnc4[nH]3)no2)C(C)C)cc1] |
| 240 | 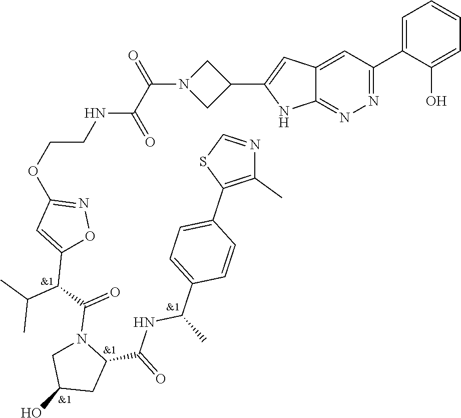 [SMILES: Cc1ncsc1-c1ccc([C@H](C)NC(=O)[C@@H]2C[C@@H](O)CN2C(=O)[C@@H](c2cc(OCCNC(=O)C(=O)N3CC(c4cc5cc(-c6ccccc6O)nnc5[nH]4)C3)no2)C(C)C)cc1] |
| 241 | 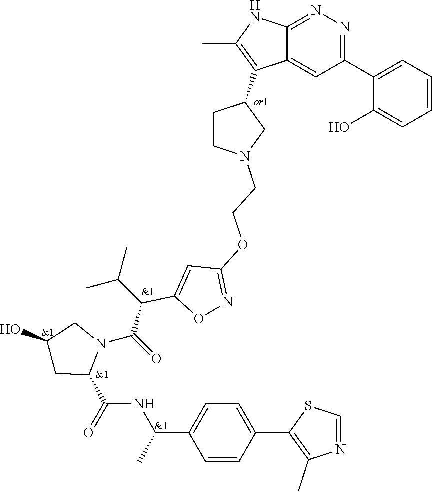 [SMILES: Cc1ncsc1-c1ccc([C@H](C)NC(=O)[C@@H]2C[C@@H](O)CN2C(=O)[C@@H](c2cc(OCCN3CC[C@H](c4c(C)[nH]c5nnc(-c6ccccc6O)cc45)C3)no2)C(C)C)cc1] |
| 242 | 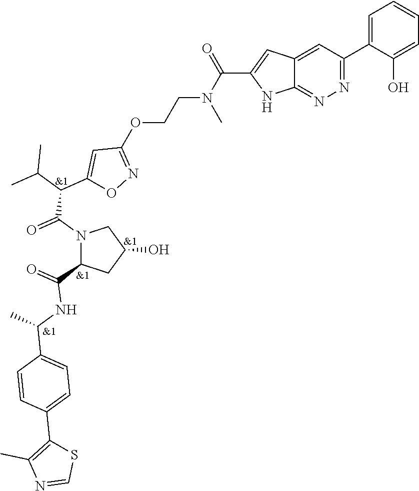 [SMILES: Cc1ncsc1-c1ccc([C@H](C)NC(=O)[C@@H]2C[C@@H](O)CN2C(=O)[C@@H](c2cc(OCCN(C)C(=O)c3cc4cc(-c5ccccc5O)nnc4[nH]3)no2)C(C)C)cc1] |
| 243 | 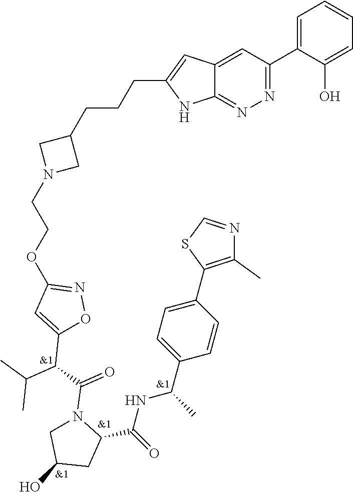 [SMILES: Cc1ncsc1-c1ccc([C@H](C)NC(=O)[C@@H]2C[C@@H](O)CN2C(=O)[C@@H](c2cc(OCCN3CC(CCCc4cc5cc(-c6ccccc6O)nnc5[nH]4)C3)no2)C(C)C)cc1] |
| 244 | 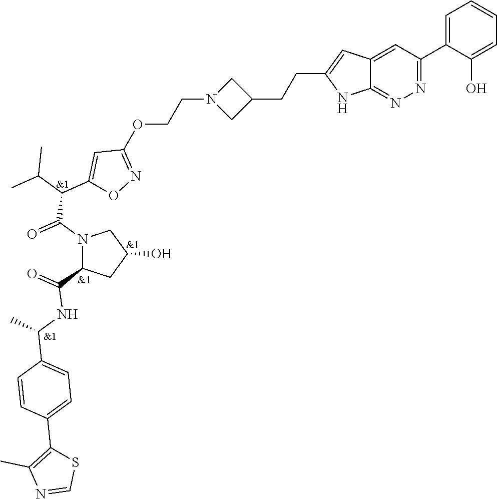 [SMILES: Cc1ncsc1-c1ccc([C@H](C)NC(=O)[C@@H]2C[C@@H](O)CN2C(=O)[C@@H](c2cc(OCCN3CC(CCc4cc5cc(-c6ccccc6O)nnc5[nH]4)C3)no2)C(C)C)cc1] |
| 245 | 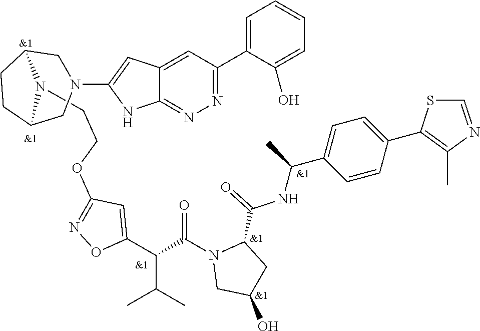 [SMILES: Cc1ncsc1-c1ccc([C@H](C)NC(=O)[C@@H]2C[C@@H](O)CN2C(=O)[C@@H](c2cc(OCCN3[C@@H]4CC[C@H]3CN(c3cc5cc(-c6ccccc6O)nnc5[nH]3)C4)no2)C(C)C)cc1] |
| 246 | 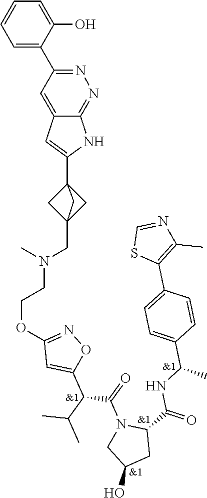 [SMILES: Cc1ncsc1-c1ccc([C@H](C)NC(=O)[C@@H]2C[C@@H](O)CN2C(=O)[C@@H](c2cc(OCCN(C)CC34CC(c5cc6cc(-c7ccccc7O)nnc6[nH]5)(C3)C4)no2)C(C)C)cc1] |
| 247 | 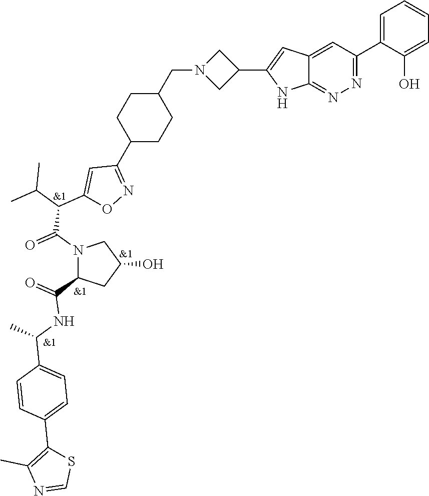 [SMILES: Cc1ncsc1-c1ccc([C@H](C)NC(=O)[C@@H]2C[C@@H](O)CN2C(=O)[C@@H](c2cc(C3CCC(CN4CC(c5cc6cc(-c7ccccc7O)nnc6[nH]5)C4)CC3)no2)C(C)C)cc1] |
| 248 | 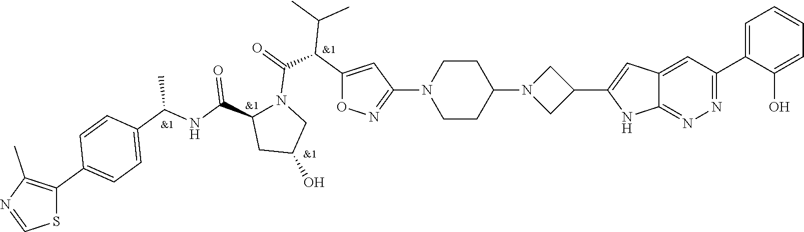 [SMILES: Cc1ncsc1-c1ccc([C@H](C)NC(=O)[C@@H]2C[C@@H](O)CN2C(=O)[C@@H](c2cc(N3CCC(N4CC(c5cc6cc(-c7ccccc7O)nnc6[nH]5)C4)CC3)no2)C(C)C)cc1] |
| 249 | 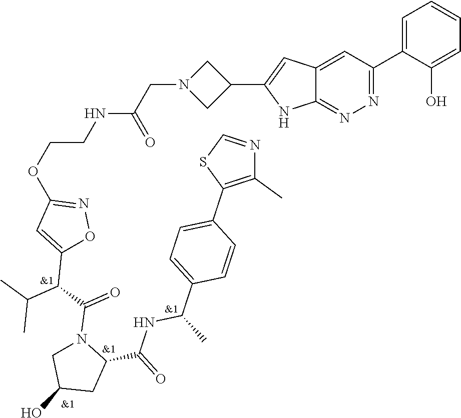 [SMILES: Cc1ncsc1-c1ccc([C@H](C)NC(=O)[C@@H]2C[C@@H](O)CN2C(=O)[C@@H](c2cc(OCCNC(=O)CN3CC(c4cc5cc(-c6ccccc6O)nnc5[nH]4)C3)no2)C(C)C)cc1] |
| 250 | 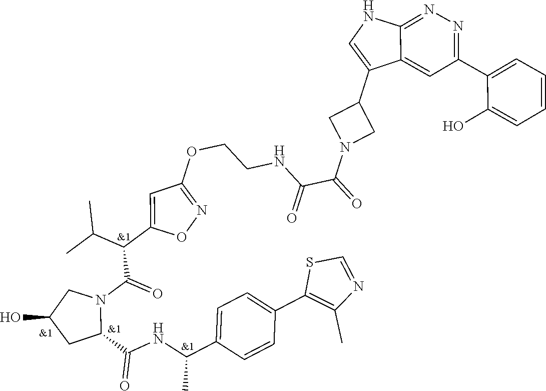 [SMILES: Cc1ncsc1-c1ccc([C@H](C)NC(=O)[C@@H]2C[C@@H](O)CN2C(=O)[C@@H](c2cc(OCCNC(=O)C(=O)N3CC(c4c[nH]c5nnc(-c6ccccc6O)cc45)C3)no2)C(C)C)cc1] |
| 251 | 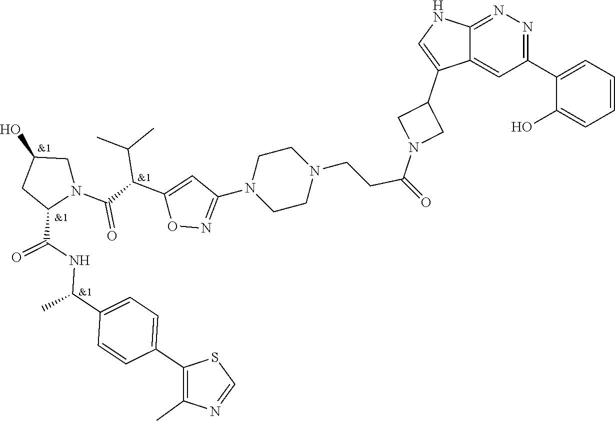 [SMILES: Cc1ncsc1-c1ccc([C@H](C)NC(=O)[C@@H]2C[C@@H](O)CN2C(=O)[C@@H](c2cc(N3CCN(CCC(=O)N4CC(c5c[nH]c6nnc(-c7ccccc7O)cc56)C4)CC3)no2)C(C)C)cc1] |
| 252 | 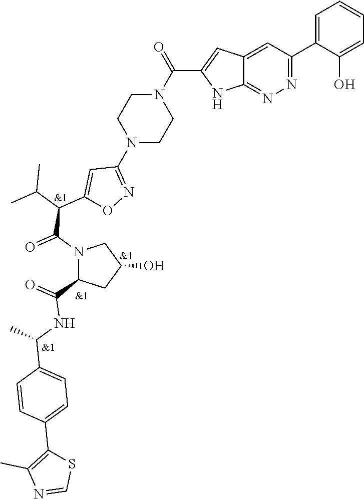 [SMILES: Cc1ncsc1-c1ccc([C@H](C)NC(=O)[C@@H]2C[C@@H](O)CN2C(=O)[C@H](c2cc(N3CCN(C(=O)c4cc5cc(-c6ccccc6O)nnc5[nH]4)CC3)no2)C(C)C)cc1] |
| 253 | 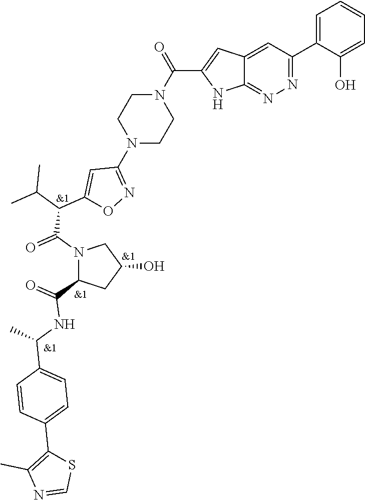 [SMILES: Cc1ncsc1-c1ccc([C@H](C)NC(=O)[C@@H]2C[C@@H](O)CN2C(=O)[C@@H](c2cc(N3CCN(C(=O)c4cc5cc(-c6ccccc6O)nnc5[nH]4)CC3)no2)C(C)C)cc1] |
| 254 | 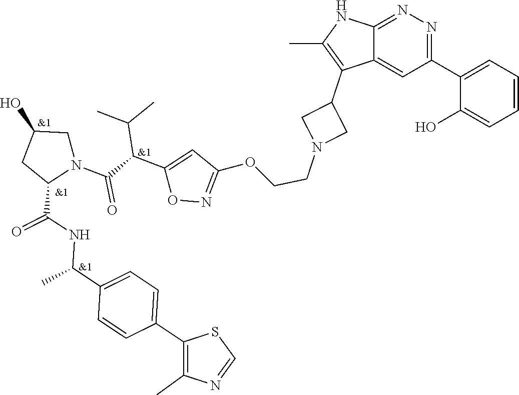 [SMILES: Cc1ncsc1-c1ccc([C@H](C)NC(=O)[C@@H]2C[C@@H](O)CN2C(=O)[C@@H](c2cc(OCCN3CC(c4c(C)[nH]c5nnc(-c6ccccc6O)cc45)C3)no2)C(C)C)cc1] |
| 255 | 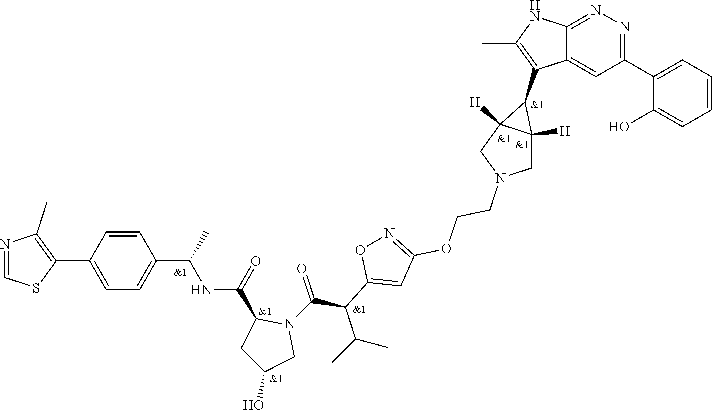 |
| 256 | 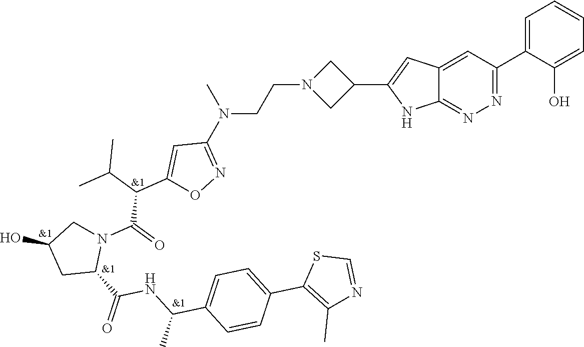 [SMILES: Cc1ncsc1-c1ccc([C@H](C)NC(=O)[C@@H]2C[C@@H](O)CN2C(=O)[C@@H](c2cc(N(C)CCN3CC(c4cc5cc(-c6ccccc6O)nnc5[nH]4)C3)no2)C(C)C)cc1] |
| 257 | 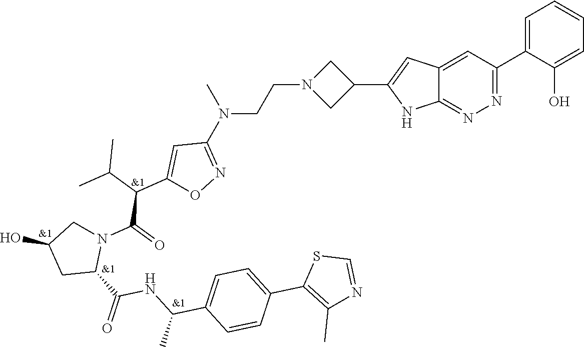 [SMILES: Cc1ncsc1-c1ccc([C@H](C)NC(=O)[C@@H]2C[C@@H](O)CN2C(=O)[C@H](c2cc(N(C)CCN3CC(c4cc5cc(-c6ccccc6O)nnc5[nH]4)C3)no2)C(C)C)cc1] |
| 258 | 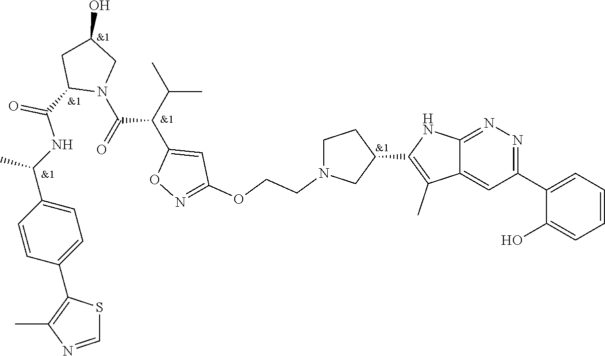 [SMILES: Cc1ncsc1-c1ccc([C@H](C)NC(=O)[C@@H]2C[C@@H](O)CN2C(=O)[C@@H](c2cc(OCCN3CC[C@H](c4[nH]c5nnc(-c6ccccc6O)cc5c4C)C3)no2)C(C)C)cc1] |
| 259 | 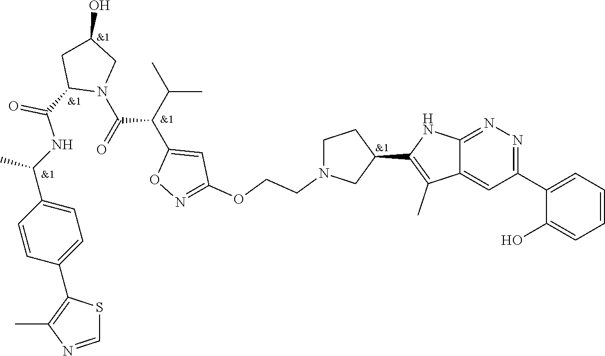 [SMILES: Cc1ncsc1-c1ccc([C@H](C)NC(=O)[C@@H]2C[C@@H](O)CN2C(=O)[C@@H](c2cc(OCCN3CC[C@@H](c4[nH]c5nnc(-c6ccccc6O)cc5c4C)C3)no2)C(C)C)cc1] |
| 260 | 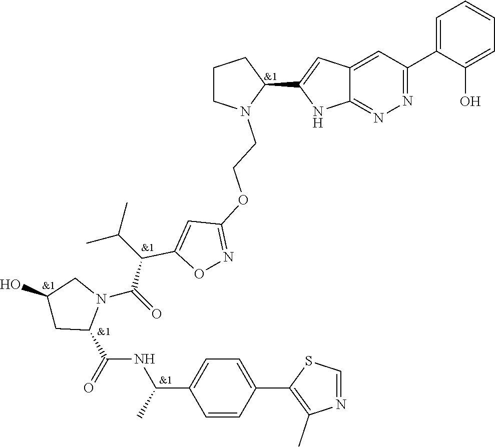 [SMILES: Cc1ncsc1-c1ccc([C@H](C)NC(=O)[C@@H]2C[C@@H](O)CN2C(=O)[C@@H](c2cc(OCCN3CCC[C@H]3c3cc4cc(-c5ccccc5O)nnc4[nH]3)no2)C(C)C)cc1] |
| 261 | 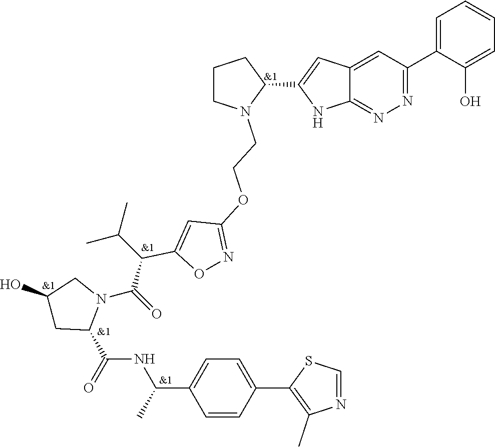 [SMILES: Cc1ncsc1-c1ccc([C@H](C)NC(=O)[C@@H]2C[C@@H](O)CN2C(=O)[C@@H](c2cc(OCCN3CCC[C@@H]3c3cc4cc(-c5ccccc5O)nnc4[nH]3)no2)C(C)C)cc1] |
| 262 | 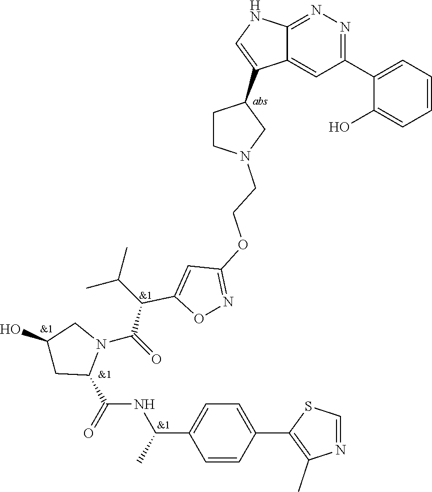 [SMILES: Cc1ncsc1-c1ccc([C@H](C)NC(=O)[C@@H]2C[C@@H](O)CN2C(=O)[C@@H](c2cc(OCCN3CC[C@@H](c4c[nH]c5nnc(-c6ccccc6O)cc45)C3)no2)C(C)C)cc1] |
| 263 | 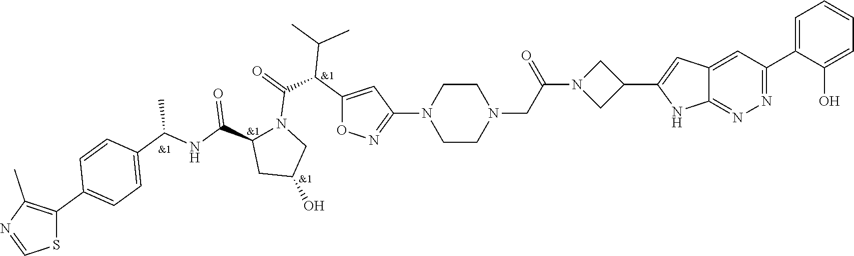 [SMILES: Cc1ncsc1-c1ccc([C@H](C)NC(=O)[C@@H]2C[C@@H](O)CN2C(=O)[C@@H](c2cc(N3CCN(CC(=O)N4CC(c5cc6cc(-c7ccccc7O)nnc6[nH]5)C4)CC3)no2)C(C)C)cc1] |
| 264 | 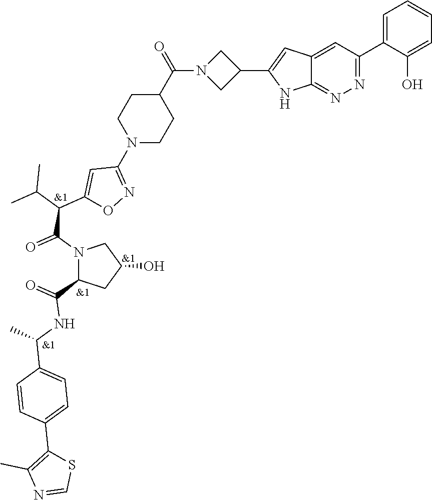 [SMILES: Cc1ncsc1-c1ccc([C@H](C)NC(=O)[C@@H]2C[C@@H](O)CN2C(=O)[C@H](c2cc(N3CCC(C(=O)N4CC(c5cc6cc(-c7ccccc7O)nnc6[nH]5)C4)CC3)no2)C(C)C)cc1] |
| 265 | 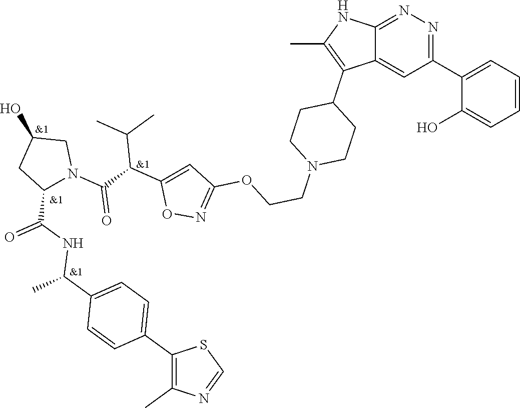 [SMILES: Cc1ncsc1-c1ccc([C@H](C)NC(=O)[C@@H]2C[C@@H](O)CN2C(=O)[C@@H](c2cc(OCCN3CCC(c4c(C)[nH]c5nnc(-c6ccccc6O)cc45)CC3)no2)C(C)C)cc1] |
| 266 | 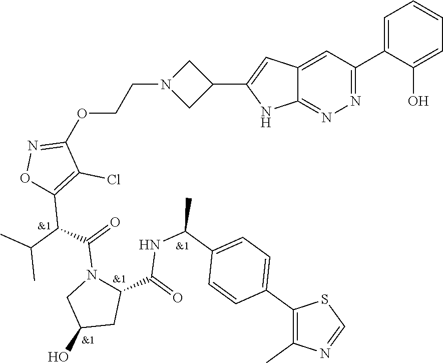 [SMILES: Cc1ncsc1-c1ccc([C@H](C)NC(=O)[C@@H]2C[C@@H](O)CN2C(=O)[C@@H](c2onc(OCCN3CC(c4cc5cc(-c6ccccc6O)nnc5[nH]4)C3)c2Cl)C(C)C)cc1] |
| 267 | 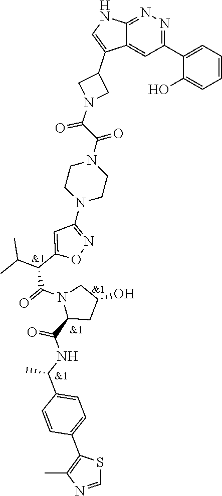 [SMILES: Cc1ncsc1-c1ccc([C@H](C)NC(=O)[C@@H]2C[C@@H](O)CN2C(=O)[C@@H](c2cc(N3CCN(C(=O)C(=O)N4CC(c5c[nH]c6nnc(-c7ccccc7O)cc56)C4)CC3)no2)C(C)C)cc1] |
| 268 | 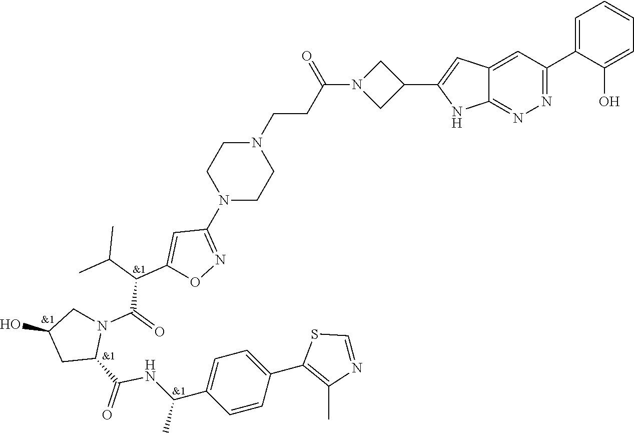 [SMILES: Cc1ncsc1-c1ccc([C@H](C)NC(=O)[C@@H]2C[C@@H](O)CN2C(=O)[C@@H](c2cc(N3CCN(CCC(=O)N4CC(c5cc6cc(-c7ccccc7O)nnc6[nH]5)C4)CC3)no2)C(C)C)cc1] |
| 269 | 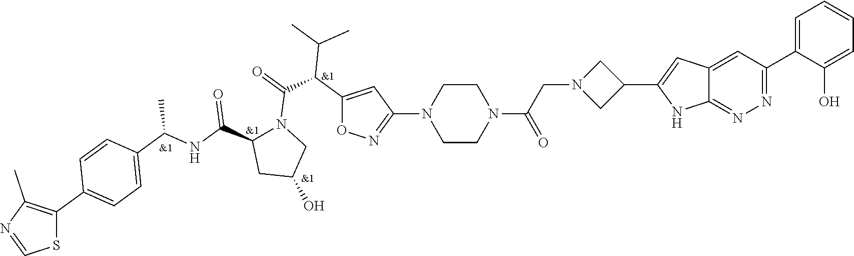 [SMILES: Cc1ncsc1-c1ccc([C@H](C)NC(=O)[C@@H]2C[C@@H](O)CN2C(=O)[C@@H](c2cc(N3CCN(C(=O)CN4CC(c5cc6cc(-c7ccccc7O)nnc6[nH]5)C4)CC3)no2)C(C)C)cc1] |
| 270 | 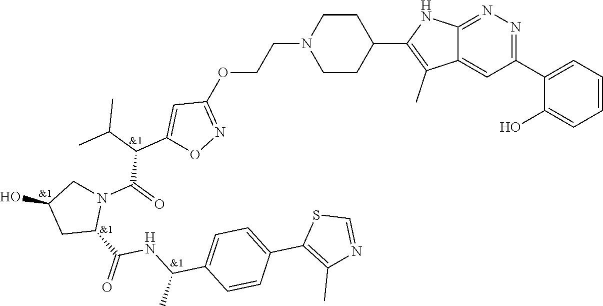 [SMILES: Cc1ncsc1-c1ccc([C@H](C)NC(=O)[C@@H]2C[C@@H](O)CN2C(=O)[C@@H](c2cc(OCCN3CCC(c4[nH]c5nnc(-c6ccccc6O)cc5c4C)CC3)no2)C(C)C)cc1] |
| 271 | 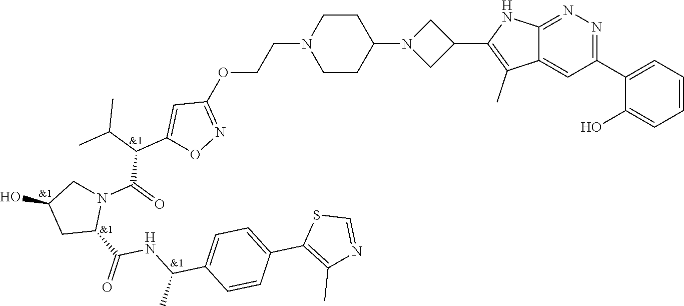 [SMILES: Cc1ncsc1-c1ccc([C@H](C)NC(=O)[C@@H]2C[C@@H](O)CN2C(=O)[C@@H](c2cc(OCCN3CCC(N4CC(c5[nH]c6nnc(-c7ccccc7O)cc6c5C)C4)CC3)no2)C(C)C)cc1] |
| 272 | 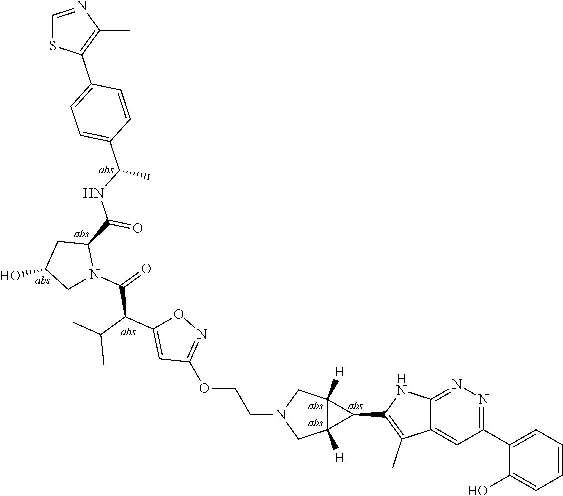 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3[nH]c4nnc(-c5ccccc5O)cc4c3C)no2)C(C)C)cc1] |
| 273 | 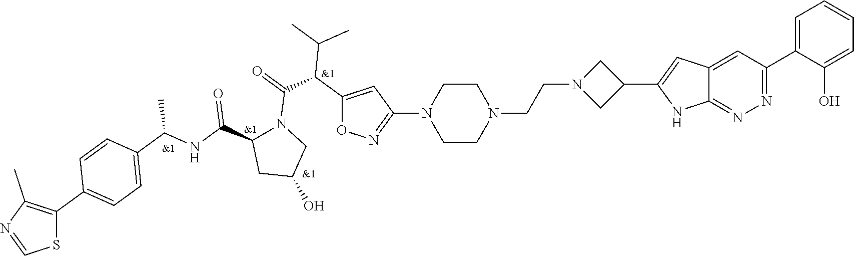 [SMILES: Cc1ncsc1-c1ccc([C@H](C)NC(=O)[C@@H]2C[C@@H](O)CN2C(=O)[C@@H](c2cc(N3CCN(CCN4CC(c5cc6cc(-c7ccccc7O)nnc6[nH]5)C4)CC3)no2)C(C)C)cc1] |
| 274 | 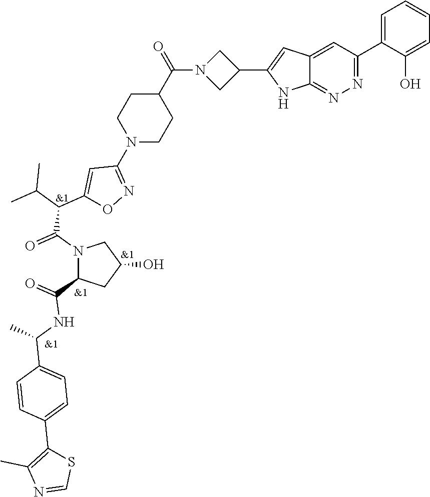 [SMILES: Cc1ncsc1-c1ccc([C@H](C)NC(=O)[C@@H]2C[C@@H](O)CN2C(=O)[C@@H](c2cc(N3CCC(C(=O)N4CC(c5cc6cc(-c7ccccc7O)nnc6[nH]5)C4)CC3)no2)C(C)C)cc1] |
| 275 | 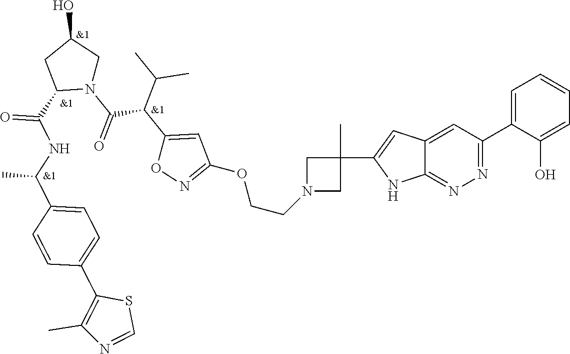 [SMILES: Cc1ncsc1-c1ccc([C@H](C)NC(=O)[C@@H]2C[C@@H](O)CN2C(=O)[C@@H](c2cc(OCCN3CC(C)(c4cc5cc(-c6ccccc6O)nnc5[nH]4)C3)no2)C(C)C)cc1] |
| 276 | 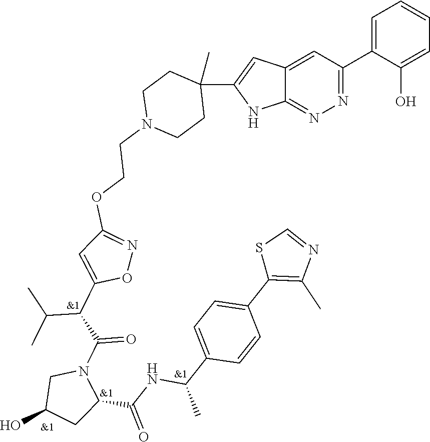 [SMILES: Cc1ncsc1-c1ccc([C@H](C)NC(=O)[C@@H]2C[C@@H](O)CN2C(=O)[C@@H](c2cc(OCCN3CCC(C)(c4cc5cc(-c6ccccc6O)nnc5[nH]4)CC3)no2)C(C)C)cc1] |
| 277 | 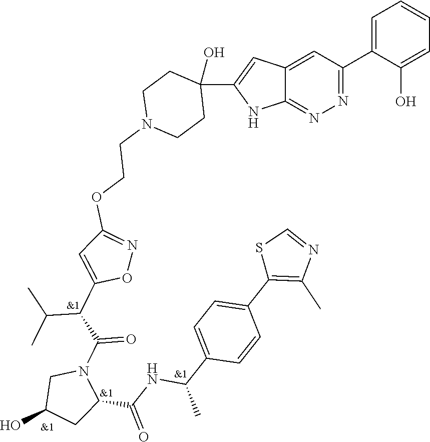 [SMILES: Cc1ncsc1-c1ccc([C@H](C)NC(=O)[C@@H]2C[C@@H](O)CN2C(=O)[C@@H](c2cc(OCCN3CCC(O)(c4cc5cc(-c6ccccc6O)nnc5[nH]4)CC3)no2)C(C)C)cc1] |
| 278 | 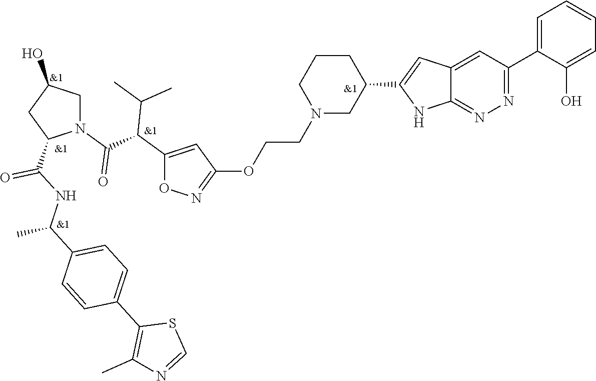 [SMILES: Cc1ncsc1-c1ccc([C@H](C)NC(=O)[C@@H]2C[C@@H](O)CN2C(=O)[C@@H](c2cc(OCCN3CCC[C@H](c4cc5cc(-c6ccccc6O)nnc5[nH]4)C3)no2)C(C)C)cc1] |
| 279 | 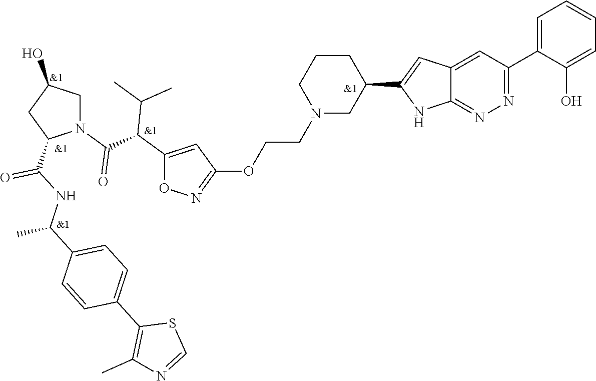 [SMILES: Cc1ncsc1-c1ccc([C@H](C)NC(=O)[C@@H]2C[C@@H](O)CN2C(=O)[C@@H](c2cc(OCCN3CCC[C@@H](c4cc5cc(-c6ccccc6O)nnc5[nH]4)C3)no2)C(C)C)cc1] |
| 280 | 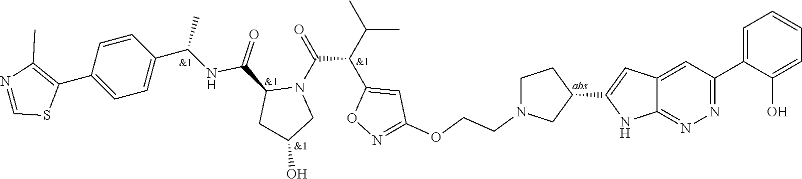 [SMILES: Cc1ncsc1-c1ccc([C@H](C)NC(=O)[C@@H]2C[C@@H](O)CN2C(=O)[C@@H](c2cc(OCCN3CC[C@H](c4cc5cc(-c6ccccc6O)nnc5[nH]4)C3)no2)C(C)C)cc1] |
| 281 | 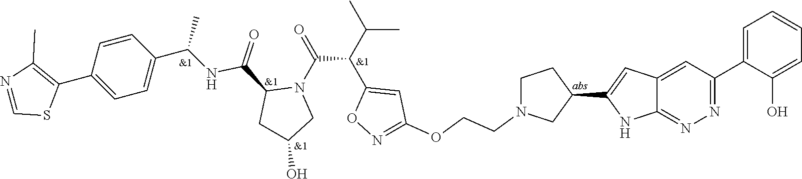 [SMILES: Cc1ncsc1-c1ccc([C@H](C)NC(=O)[C@@H]2C[C@@H](O)CN2C(=O)[C@@H](c2cc(OCCN3CC[C@@H](c4cc5cc(-c6ccccc6O)nnc5[nH]4)C3)no2)C(C)C)cc1] |
| 282 | 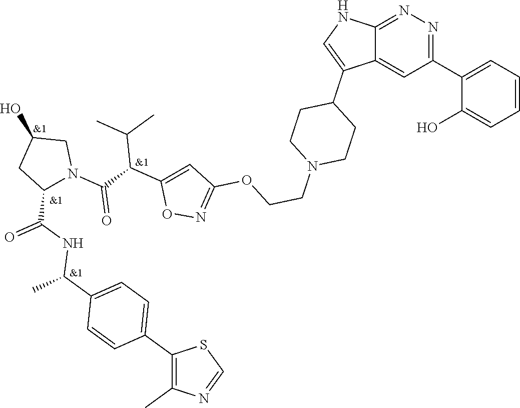 [SMILES: Cc1ncsc1-c1ccc([C@H](C)NC(=O)[C@@H]2C[C@@H](O)CN2C(=O)[C@@H](c2cc(OCCN3CCC(c4c[nH]c5nnc(-c6ccccc6O)cc45)CC3)no2)C(C)C)cc1] |
| 283 | 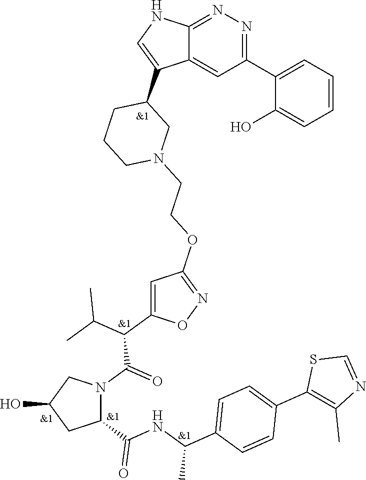 [SMILES: Cc1ncsc1-c1ccc([C@H](C)NC(=O)[C@@H]2C[C@@H](O)CN2C(=O)[C@@H](c2cc(OCCN3CCC[C@@H](c4c[nH]c5nnc(-c6ccccc6O)cc45)C3)no2)C(C)C)cc1] |
| 284 | 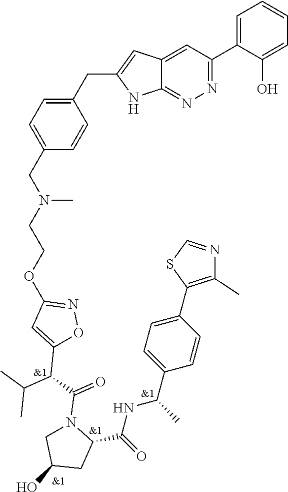 [SMILES: Cc1ncsc1-c1ccc([C@H](C)NC(=O)[C@@H]2C[C@@H](O)CN2C(=O)[C@@H](c2cc(OCCN(C)Cc3ccc(Cc4cc5cc(-c6ccccc6O)nnc5[nH]4)cc3)no2)C(C)C)cc1] |
| 285 | 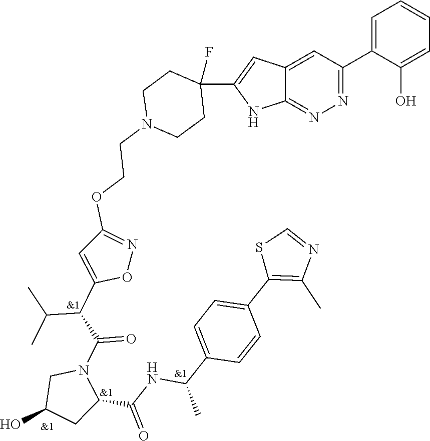 [SMILES: Cc1ncsc1-c1ccc([C@H](C)NC(=O)[C@@H]2C[C@@H](O)CN2C(=O)[C@@H](c2cc(OCCN3CCC(F)(c4cc5cc(-c6ccccc6O)nnc5[nH]4)CC3)no2)C(C)C)cc1] |
| 286 | 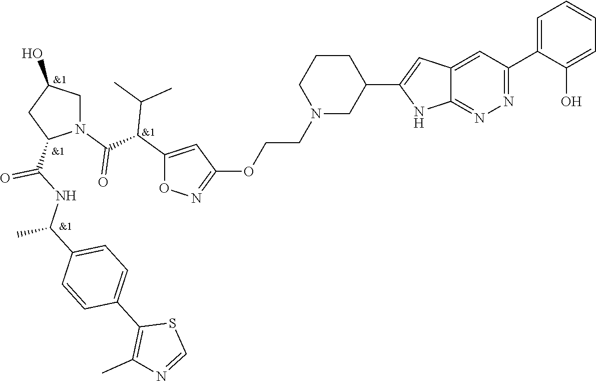 [SMILES: Cc1ncsc1-c1ccc([C@H](C)NC(=O)[C@@H]2C[C@@H](O)CN2C(=O)[C@@H](c2cc(OCCN3CCCC(c4cc5cc(-c6ccccc6O)nnc5[nH]4)C3)no2)C(C)C)cc1] |
| 287 | 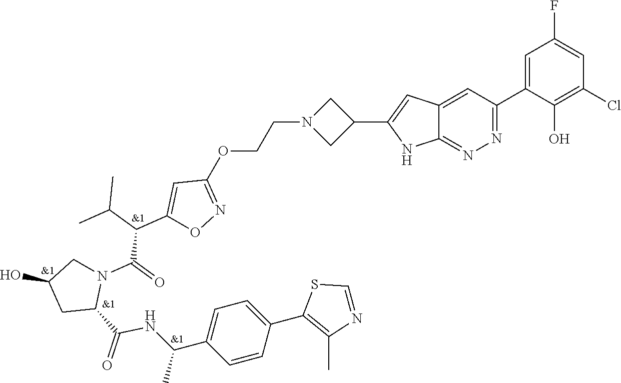 [SMILES: Cc1ncsc1-c1ccc([C@H](C)NC(=O)[C@@H]2C[C@@H](O)CN2C(=O)[C@@H](c2cc(OCCN3CC(c4cc5cc(-c6cc(F)cc(Cl)c6O)nnc5[nH]4)C3)no2)C(C)C)cc1] |
| 288 | 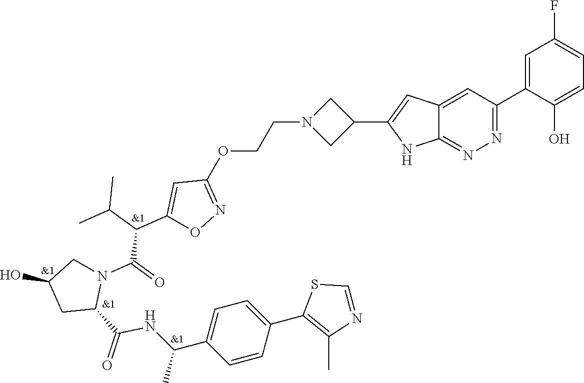 [SMILES: Cc1ncsc1-c1ccc([C@H](C)NC(=O)[C@@H]2C[C@@H](O)CN2C(=O)[C@@H](c2cc(OCCN3CC(c4cc5cc(-c6cc(F)ccc6O)nnc5[nH]4)C3)no2)C(C)C)cc1] |
| 289 | 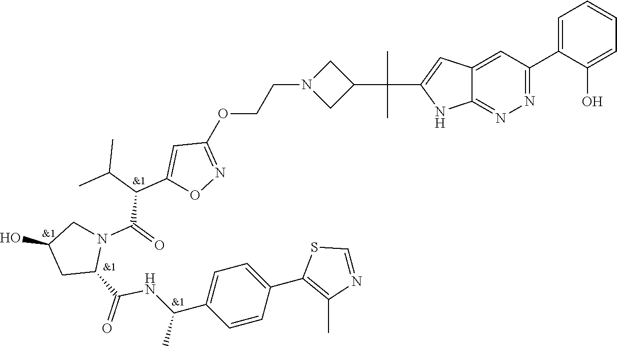 [SMILES: Cc1ncsc1-c1ccc([C@H](C)NC(=O)[C@@H]2C[C@@H](O)CN2C(=O)[C@@H](c2cc(OCCN3CC(C(C)(C)c4cc5cc(-c6ccccc6O)nnc5[nH]4)C3)no2)C(C)C)cc1] |
| 290 | 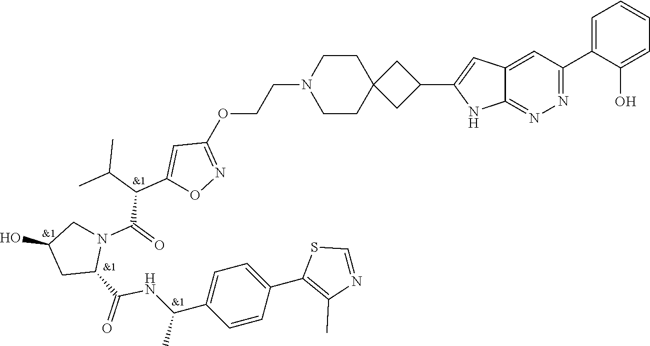 [SMILES: Cc1ncsc1-c1ccc([C@H](C)NC(=O)[C@@H]2C[C@@H](O)CN2C(=O)[C@@H](c2cc(OCCN3CCC4(CC3)CC(c3cc5cc(-c6ccccc6O)nnc5[nH]3)C4)no2)C(C)C)cc1] |
| 291 | 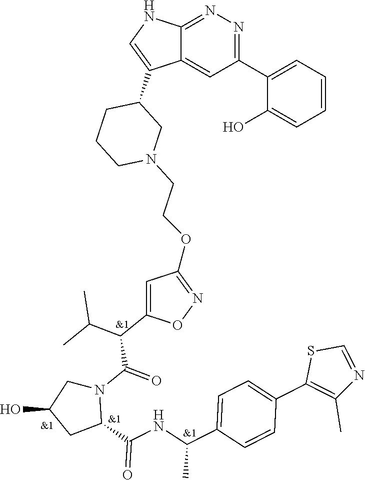 [SMILES: Cc1ncsc1-c1ccc([C@H](C)NC(=O)[C@@H]2C[C@@H](O)CN2C(=O)[C@@H](c2cc(OCCN3CCC[C@H](c4c[nH]c5nnc(-c6ccccc6O)cc45)C3)no2)C(C)C)cc1] |
| 292 | 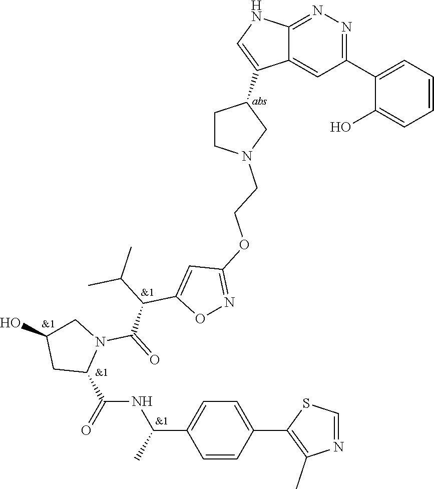 [SMILES: Cc1ncsc1-c1ccc([C@H](C)NC(=O)[C@@H]2C[C@@H](O)CN2C(=O)[C@@H](c2cc(OCCN3CC[C@H](c4c[nH]c5nnc(-c6ccccc6O)cc45)C3)no2)C(C)C)cc1] |
| 293 | 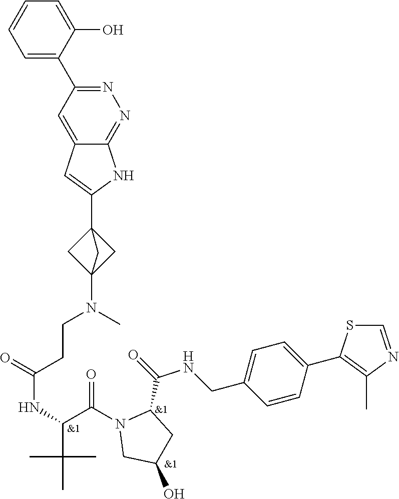 [SMILES: Cc1ncsc1-c1ccc(CNC(=O)[C@@H]2C[C@@H](O)CN2C(=O)[C@@H](NC(=O)CCN(C)C23CC(c4cc5cc(-c6ccccc6O)nnc5[nH]4)(C2)C3)C(C)(C)C)cc1] |
| 294 | 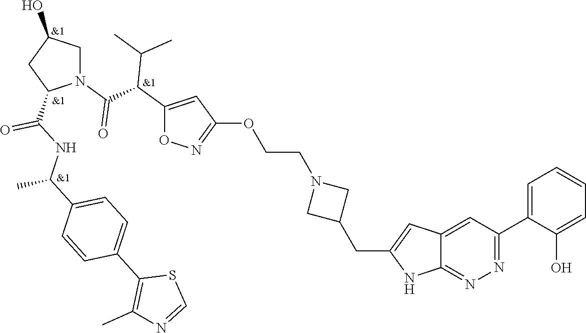 [SMILES: Cc1ncsc1-c1ccc([C@H](C)NC(=O)[C@@H]2C[C@@H](O)CN2C(=O)[C@@H](c2cc(OCCN3CC(Cc4cc5cc(-c6ccccc6O)nnc5[nH]4)C3)no2)C(C)C)cc1] |
| 295 | 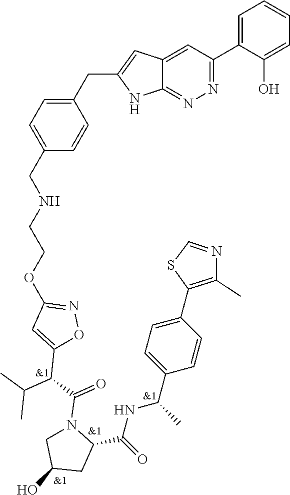 [SMILES: Cc1ncsc1-c1ccc([C@H](C)NC(=O)[C@@H]2C[C@@H](O)CN2C(=O)[C@@H](c2cc(OCCNCc3ccc(Cc4cc5cc(-c6ccccc6O)nnc5[nH]4)cc3)no2)C(C)C)cc1] |
| 296 | 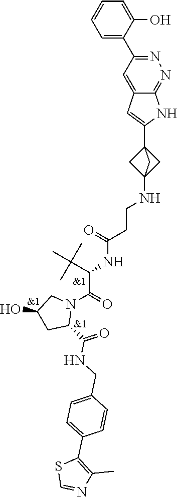 [SMILES: Cc1ncsc1-c1ccc(CNC(=O)[C@@H]2C[C@@H](O)CN2C(=O)[C@@H](NC(=O)CCNC23CC(c4cc5cc(-c6ccccc6O)nnc5[nH]4)(C2)C3)C(C)(C)C)cc1] |
| 297 | 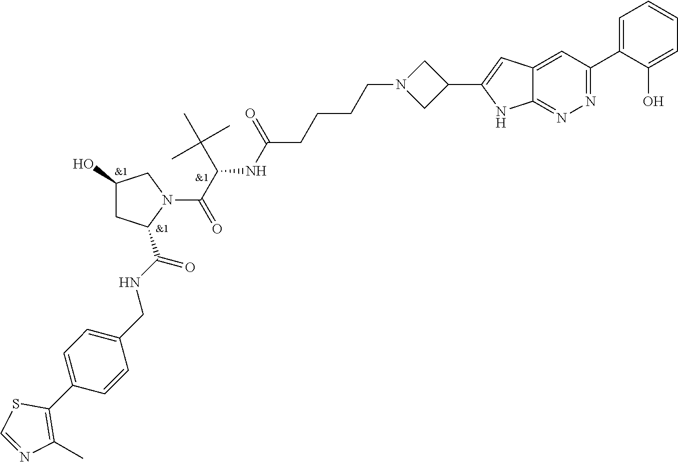 [SMILES: Cc1ncsc1-c1ccc(CNC(=O)[C@@H]2C[C@@H](O)CN2C(=O)[C@@H](NC(=O)CCCCN2CC(c3cc4cc(-c5ccccc5O)nnc4[nH]3)C2)C(C)(C)C)cc1] |
| 298 | 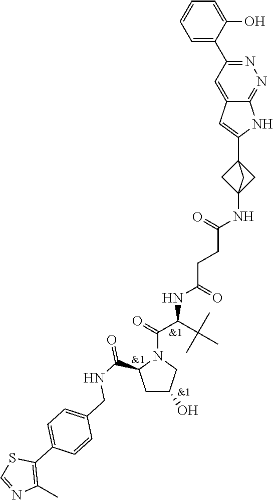 [SMILES: Cc1ncsc1-c1ccc(CNC(=O)[C@@H]2C[C@@H](O)CN2C(=O)[C@@H](NC(=O)CCC(=O)NC23CC(c4cc5cc(-c6ccccc6O)nnc5[nH]4)(C2)C3)C(C)(C)C)cc1] |
| 299 | 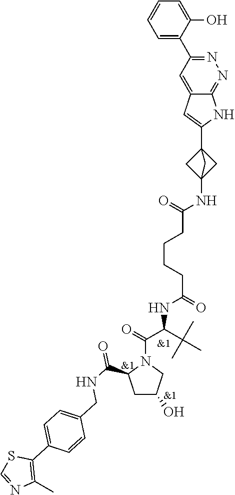 [SMILES: Cc1ncsc1-c1ccc(CNC(=O)[C@@H]2C[C@@H](O)CN2C(=O)[C@@H](NC(=O)CCCCC(=O)NC23CC(c4cc5cc(-c6ccccc6O)nnc5[nH]4)(C2)C3)C(C)(C)C)cc1] |
| 300 | 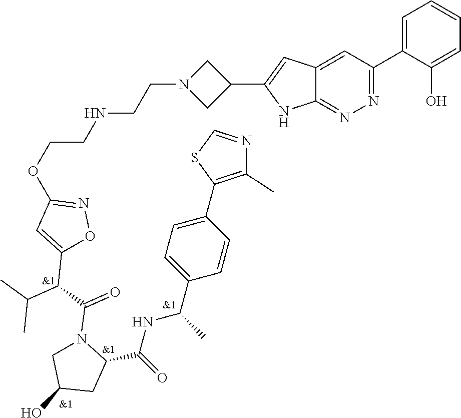 [SMILES: Cc1ncsc1-c1ccc([C@H](C)NC(=O)[C@@H]2C[C@@H](O)CN2C(=O)[C@@H](c2cc(OCCNCCN3CC(c4cc5cc(-c6ccccc6O)nnc5[nH]4)C3)no2)C(C)C)cc1] |
| 301 | 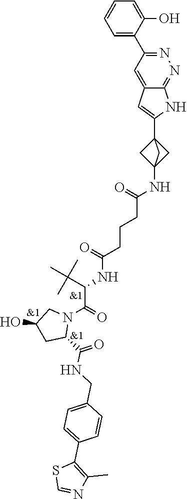 [SMILES: Cc1ncsc1-c1ccc(CNC(=O)[C@@H]2C[C@@H](O)CN2C(=O)[C@@H](NC(=O)CCCC(=O)NC23CC(c4cc5cc(-c6ccccc6O)nnc5[nH]4)(C2)C3)C(C)(C)C)cc1] |
| 302 | 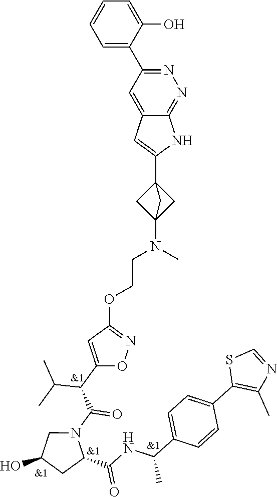 [SMILES: Cc1ncsc1-c1ccc([C@H](C)NC(=O)[C@@H]2C[C@@H](O)CN2C(=O)[C@@H](c2cc(OCCN(C)C34CC(c5cc6cc(-c7ccccc7O)nnc6[nH]5)(C3)C4)no2)C(C)C)cc1] |
| 303 | 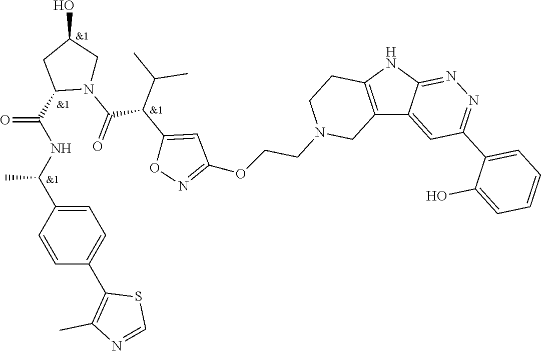 [SMILES: Cc1ncsc1-c1ccc([C@H](C)NC(=O)[C@@H]2C[C@@H](O)CN2C(=O)[C@@H](c2cc(OCCN3CCc4[nH]c5nnc(-c6ccccc6O)cc5c4C3)no2)C(C)C)cc1] |
| 304 | 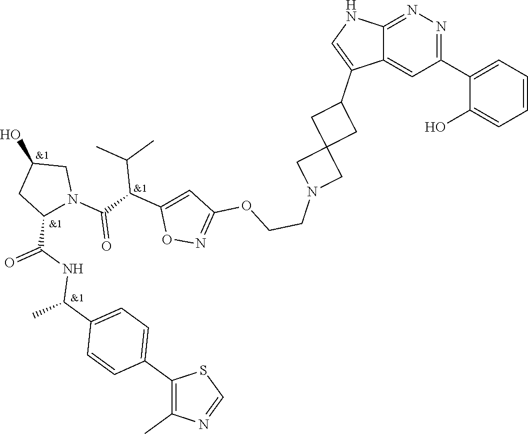 [SMILES: Cc1ncsc1-c1ccc([C@H](C)NC(=O)[C@@H]2C[C@@H](O)CN2C(=O)[C@@H](c2cc(OCCN3CC4(CC(c5c[nH]c6nnc(-c7ccccc7O)cc56)C4)C3)no2)C(C)C)cc1] |
| 305 | 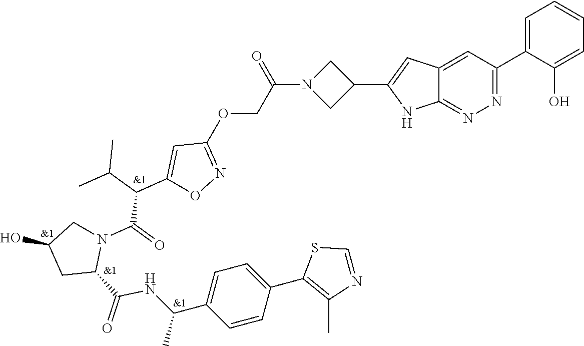 [SMILES: Cc1ncsc1-c1ccc([C@H](C)NC(=O)[C@@H]2C[C@@H](O)CN2C(=O)[C@@H](c2cc(OCC(=O)N3CC(c4cc5cc(-c6ccccc6O)nnc5[nH]4)C3)no2)C(C)C)cc1] |
| 306 | 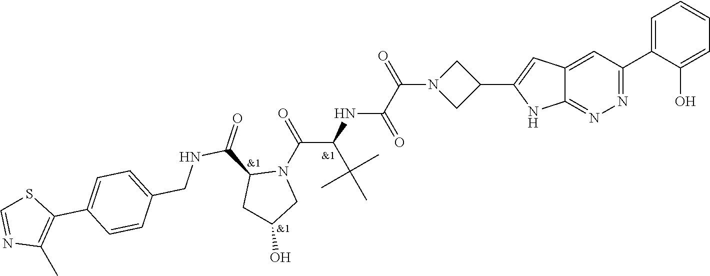 [SMILES: Cc1ncsc1-c1ccc(CNC(=O)[C@@H]2C[C@@H](O)CN2C(=O)[C@@H](NC(=O)C(=O)N2CC(c3cc4cc(-c5ccccc5O)nnc4[nH]3)C2)C(C)(C)C)cc1] |
| 307 | 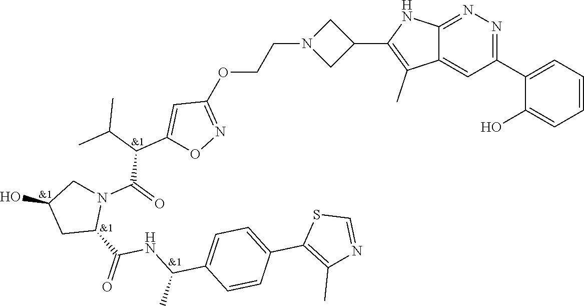 [SMILES: Cc1ncsc1-c1ccc([C@H](C)NC(=O)[C@@H]2C[C@@H](O)CN2C(=O)[C@@H](c2cc(OCCN3CC(c4[nH]c5nnc(-c6ccccc6O)cc5c4C)C3)no2)C(C)C)cc1] |
| 308 | 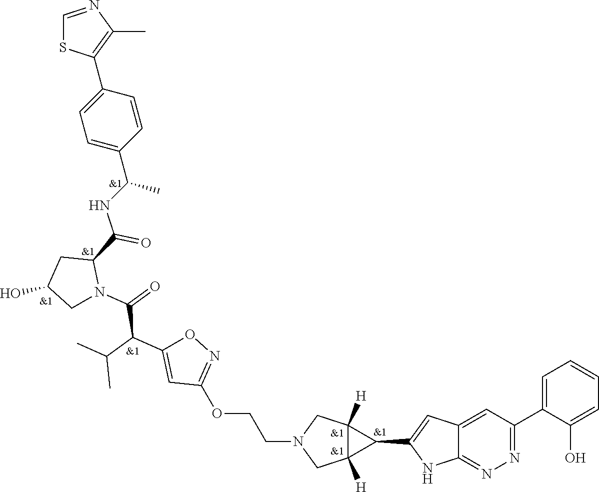 [SMILES: Cc1ncsc1-c1ccc([C@H](C)NC(=O)[C@@H]2C[C@@H](O)CN2C(=O)[C@@H](c2cc(OCCN3C[C@@H]4[C@H](C3)[C@H]4c3cc4cc(-c5ccccc5O)nnc4[nH]3)no2)C(C)C)cc1] |
| 309 | 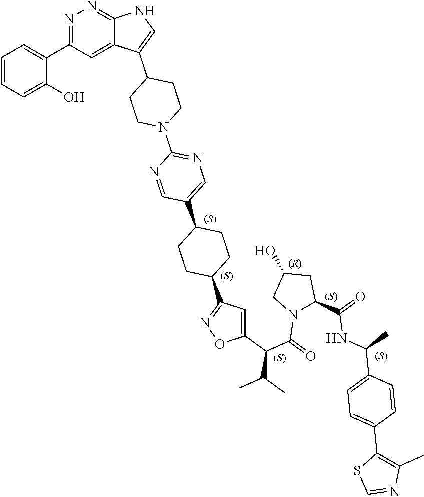 |
| 310 | 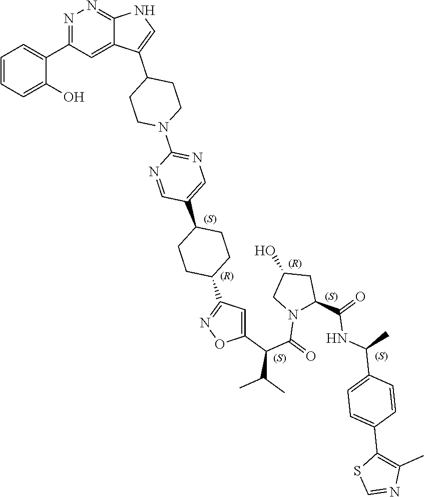 |

In the table above, the double, dashed bonds above indicate π-aromatic bonds.

In some embodiments, the compound has a ratio of BRG1 IC₅₀ to BRM IC₅₀ of at least 5. In some embodiments, the compound has a ratio of BRG1 IC₅₀ to BRM IC₅₀ of at least 7. In some embodiments, the compound has a ratio of BRG1 IC₅₀ to BRM IC₅₀ of at least 10. In some embodiments, the compound has a ratio of BRG1 IC₅₀ to BRM IC₅₀ of at least 15. In some embodiments, the compound has a ratio of BRG1 IC₅₀ to BRM IC₅₀ of at least 20. In some embodiments, the compound has a ratio of BRG1 IC₅₀ to BRM IC₅₀ of at least 25. In some embodiments, the compound has a ratio of BRG1 IC₅₀ to BRM IC₅₀ of at least 30.

In an aspect, the invention features a pharmaceutical composition comprising any of the foregoing compounds and a pharmaceutically acceptable excipient.

In another aspect, the invention features a method of decreasing the activity of a BAF complex in a cell, the method involving contacting the cell with an effective amount of any of the foregoing compounds or a pharmaceutical composition thereof.

In some embodiments, the cell is a cancer cell.

In another aspect, the invention features a method of treating a BAF complex-related disorder in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the BAF complex-related disorder is cancer.

In a further aspect, the invention features a method of inhibiting BRM, the method involving contacting a cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In another aspect, the invention features a method of inhibiting BRG1, the method involving contacting the cell with an effective amount of any of the foregoing compounds or a pharmaceutical composition thereof.

In some embodiments, the cell is a cancer cell.

In a further aspect, the invention features a method of inhibiting BRM and BRG1, the method involving contacting the cell with an effective amount of any of the foregoing compounds or a pharmaceutical composition thereof.

In some embodiments, the cell is a cancer cell.

In another aspect, the invention features a method of treating a disorder related to a BRG1 loss of function mutation in a subject in need thereof, the method involving administering to the subject an effective amount of any of the foregoing compounds (e.g., a BRM/BRG1 dual inhibitor compound or a BRM-selective compound) or a pharmaceutical composition thereof.

In some embodiments, the disorder related to a BRG1 loss of function mutation is cancer. In other embodiments, the subject is determined to have a BRG1 loss of function disorder, for example, is determined to have a BRG1 loss of function cancer (for example, the cancer has been determined to include cancer cells with loss of BRG1 function).

In another aspect, the invention features a method of inducing apoptosis in a cell, the method involving contacting the cell with an effective amount of any of the foregoing compounds (e.g., a BRM/BRG1 dual inhibitor compound or a BRM-selective compound) or a pharmaceutical composition thereof.

In some embodiments, the cell is a cancer cell.

In a further aspect, the invention features a method of treating cancer in a subject in need thereof, the method including administering to the subject an effective amount of any of the foregoing compounds (e.g., a BRM/BRG1 dual inhibitor compound or a BRM-selective compound) or a pharmaceutical composition thereof.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments of any of the foregoing methods, the cancer is non-small cell lung cancer, colorectal cancer, bladder cancer, cancer of unknown primary, glioma, breast cancer, melanoma, non-melanoma skin cancer, endometrial cancer, or penile cancer.

In some embodiments of any of the foregoing methods, the cancer is a drug resistant cancer or has failed to respond to a prior therapy (e.g., vemurafenib, dacarbazine, a CTLA4 inhibitor, a PD1 inhibitor, interferon therapy, a BRAF inhibitor, a MEK inhibitor, radiotherapy, temozolomide, irinotecan, a CAR-T therapy, Herceptin®, Perjeta®, tamoxifen, Xeloda®, docetaxol, platinum agents such as carboplatin, taxanes such as paclitaxel and docetaxel, ALK inhibitors, MET inhibitors, Alimta®, Abraxane®, Adriamycin®, gemcitabine, Avastin®, Halaven®, neratinib, a PARP inhibitor, ARN810, an mTOR inhibitor, topotecan, Gemzar®, a VEGFR2 inhibitor, a folate receptor antagonist, demcizumab, fosbretabulin, or a PDL1 inhibitor).

In some embodiments of any of the foregoing methods, the cancer has or has been determined to have BRG1 mutations. In some embodiments of any of the foregoing methods, the BRG1 mutations are homozygous. In some embodiments of any of the foregoing methods, the cancer does not have, or has been determined not to have, an epidermal growth factor receptor (EGFR) mutation. In some embodiments of any of the foregoing methods, the cancer does not have, or has been determined not to have, an anaplastic lymphoma kinase (ALK) driver mutation. In some embodiments of any of the foregoing methods, the cancer has, or has been determined to have, a KRAS mutation. In some embodiments of any of the foregoing methods, the BRG1 mutation is in the ATPase catalytic domain of the protein. In some embodiments of any of the foregoing methods, the BRG1 mutation is a deletion at the C-terminus of BRG1.

In another aspect, the disclosure provides a method treating a disorder related to BAF (e.g., cancer or viral infections) in a subject in need thereof. This method includes contacting a cell with an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the disorder is a viral infection is an infection with a virus of the Retroviridae family such as the lentiviruses (e.g., Human immunodeficiency virus (HIV) and deltaretroviruses (e.g., human T cell leukemia virus I (HTLV-I), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), Togaviridae family (e.g., Rubella virus). In some embodiments, the disorder is Coffin Siris, Neurofibromatosis (e.g., NF-1, NF-2, or Schwannomatosis), or Multiple Meningioma.

In another aspect, the disclosure provides a method for treating a viral infection in a subject in need thereof. This method includes administering to the subject an effective amount of any of the foregoing compounds (e.g., a BRM/BRG1 dual inhibitor compound or a BRM-selective compound), or pharmaceutically acceptable salts thereof, or any of the foregoing pharmaceutical compositions. In some embodiments, the viral infection is an infection with a virus of the Retroviridae family such as the lentiviruses (e.g., Human immunodeficiency virus (HIV) and deltaretroviruses (e.g., human T cell leukemia virus I (HTLV-1), human T cell leukemia virus II (HTLV-II)), Hepadnaviridae family (e.g., hepatitis B virus (HBV)), Flaviviridae family (e.g., hepatitis C virus (HCV)), Adenoviridae family (e.g., Human Adenovirus), Herpesviridae family (e.g., Human cytomegalovirus (HCMV), Epstein-Barr virus, herpes simplex virus 1 (HSV-1), herpes simplex virus 2 (HSV-2), human herpesvirus 6 (HHV-6), Herpesvitus K*, CMV, varicella-zoster virus), Papillomaviridae family (e.g., Human Papillomavirus (HPV, HPV E1)), Parvoviridae family (e.g., Parvovirus B19), Polyomaviridae family (e.g., JC virus and BK virus), Paramyxoviridae family (e.g., Measles virus), or Togaviridae family (e.g., Rubella virus).

In some embodiments of any of the foregoing aspects, the compound is a BRM-selective compound. In some embodiments, the BRM-selective compound inhibits the level and/or activity of BRM at least 10-fold greater than the compound inhibits the level and/or activity of BRG1 and/or the compound binds to BRM at least 10-fold greater than the compound binds to BRG1. For example, in some embodiments, a BRM-selective compound has an IC₅₀ or IP₅₀ that is at least 10-fold lower than the IC₅₀ or IP₅₀ against BRG1. In some embodiments of any of the foregoing aspects, the compound is a BRM/BRG1 dual inhibitor compound. In some embodiments, the BRM/BRG1 dual inhibitor compound has similar activity against both BRM and BRG1 (e.g., the activity of the compound against BRM and BRG1 with within 10-fold (e.g., less than 5-fold, less than 2-fold). In some embodiments, the activity of the BRM/BRG1 dual inhibitor compound is greater against BRM. In some embodiments, the activity of the BRM/BRG1 dual inhibitor compound is greater against BRG1. For example, in some embodiments, a BRM/BRG1 dual inhibitor compound has an IC₅₀ or IP₅₀ against BRM that is within 10-fold of the IC₅₀ or IP₅₀ against BRG1.

In another aspect, the invention features a method of treating melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of reducing tumor growth of melanoma, prostate cancer, breast cancer, bone cancer, renal cell carcinoma, or a hematologic cancer in a subject in need thereof, the method including administering to the subject an effective amount of any of the foregoing compounds or pharmaceutical compositions thereof.

In another aspect, the invention features a method of suppressing metastatic progression of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of suppressing metastatic colonization of melanoma, prostate cancer, breast cancer, bone cancer, renal cell carcinoma, or a hematologic cancer in a subject, the method including administering an effective amount of any of the foregoing compounds or pharmaceutical compositions thereof.

In another aspect, the invention features a method of reducing the level and/or activity of BRG1 and/or BRM in a melanoma, prostate cancer, breast cancer, bone cancer, renal cell carcinoma, or hematologic cancer cell, the method including contacting the cell with an effective amount of any of the foregoing compounds or pharmaceutical compositions thereof.

In some embodiments of any of the above aspects, the melanoma, prostate cancer, breast cancer, bone cancer, renal cell carcinoma, or hematologic cell is in a subject.

In some embodiments of any of the above aspects, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRG1 by at least 50% (e.g., 55%, 60%, 65%, 70%, 75%, 80%, 85%, 90%, or 95%) as compared to a reference. In some embodiments, the effective amount of the compound that reduces the level and/or activity of BRG1 by at least 90% (e.g., 91%, 92%, 93%, 94%, 95%, 96%, 97%, 98%, or 99%).

In some embodiments, the effective amount of the compound reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRG1 by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments of any of the above aspects, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference. In some embodiments, the effective amount of the compound that reduces the level and/or activity of BRM by at least 50% (e.g., 55%, 60%, 65%, 70%, 75%, 80%, 85%, 90%, or 95%) as compared to a reference. In some embodiments, the effective amount of the compound that reduces the level and/or activity of BRM by at least 90% (e.g., 91%, 92%, 93%, 94%, 95%, 96%, 97%, 98%, or 99%).

In some embodiments, the effective amount of the compound reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 12 hours (e.g., 14 hours, 16 hours, 18 hours, 20 hours, 22 hours, 24 hours, 30 hours, 36 hours, 48 hours, 72 hours, or more). In some embodiments, the effective amount of the compound that reduces the level and/or activity of BRM by at least 5% (e.g., 6%, 7%, 8%, 9%, 10%, 15%, 20%, 25%, 30%, 35%, 40%, 45%, 50%, 55%, 60%, 65%, 70%, 75%, 80%, 85%, 90%, or 95%) as compared to a reference for at least 4 days (e.g., 5 days, 6 days, 7 days, 14 days, 28 days, or more).

In some embodiments, the subject has cancer. In some embodiments, the cancer expresses BRG1 and/or BRM protein and/or the cell or subject has been identified as expressing BRG1 and/or BRM. In some embodiments, the cancer expresses BRG1 protein and/or the cell or subject has been identified as expressing BRG1. In some embodiments, the cancer expresses BRM protein and/or the cell or subject has been identified as expressing BRM. In some embodiments, the cancer is melanoma (e.g., uveal melanoma, mucosal melanoma, or cutaneous melanoma). In some embodiments, the cancer is prostate cancer. In some embodiments, the cancer is a hematologic cancer, e.g., multiple myeloma, large cell lymphoma, acute T-cell leukemia, acute myeloid leukemia, myelodysplastic syndrome, immunoglobulin A lambda myeloma, diffuse mixed histiocytic and lymphocytic lymphoma, B-cell lymphoma, acute lymphoblastic leukemia (e.g., T-cell acute lymphoblastic leukemia or B-cell acute lymphoblastic leukemia), diffuse large cell lymphoma, or non-Hodgkin's lymphoma. In some embodiments, the cancer is breast cancer (e.g., an ER positive breast cancer, an ER negative breast cancer, triple positive breast cancer, or triple negative breast cancer). In some embodiments, the cancer is a bone cancer (e.g., Ewing's sarcoma). In some embodiments, the cancer is a renal cell carcinoma (e.g., a Microphthalmia Transcription Factor (MITF) family translocation renal cell carcinoma (tRCC)). In some embodiments, the cancer is metastatic (e.g., the cancer has spread to the liver). The metastatic cancer can include cells exhibiting migration and/or invasion of migrating cells and/or include cells exhibiting endothelial recruitment and/or angiogenesis. In other embodiments, the migrating cancer is a cell migration cancer. In still other embodiments, the cell migration cancer is a non-metastatic cell migration cancer. The metastatic cancer can be a cancer spread via seeding the surface of the peritoneal, pleural, pericardial, or subarachnoid spaces. Alternatively, the metastatic cancer can be a cancer spread via the lymphatic system, or a cancer spread hematogenously. In some embodiments, the effective amount of an agent that reduces the level and/or activity of BRG1 and/or BRM is an amount effective to inhibit metastatic colonization of the cancer to the liver.

In some embodiments the cancer harbors a mutation in GNAQ. In some embodiments the cancer harbors a mutation in GNA11. In some embodiments the cancer harbors a mutation in PLCB4. In some embodiments the cancer harbors a mutation in CYSLTR2. In some embodiments the cancer harbors a mutation in BAP1. In some embodiments the cancer harbors a mutation in SF3B¹. In some embodiments the cancer harbors a mutation in EIF1AX. In some embodiments the cancer harbors a TFE3 translocation. In some embodiments the cancer harbors a TFEB translocation. In some embodiments the cancer harbors a MITF translocation. In some embodiments the cancer harbors an EZH2 mutation. In some embodiments the cancer harbors a SUZ12 mutation. In some embodiments the cancer harbors an EED mutation.

In some embodiments, the method further includes administering to the subject or contacting the cell with an anticancer therapy, e.g., a chemotherapeutic or cytotoxic agent, immunotherapy, surgery, radiotherapy, thermotherapy, or photocoagulation. In some embodiments, the anticancer therapy is a chemotherapeutic or cytotoxic agent, e.g., an antimetabolite, antimitotic, antitumor antibiotic, asparagine-specific enzyme, bisphosphonates, antineoplastic, alkylating agent, DNA-Repair enzyme inhibitor, histone deacetylase inhibitor, corticosteroid, demethylating agent, immunomodulatory, janus-associated kinase inhibitor, phosphinositide 3-kinase inhibitor, proteasome inhibitor, or tyrosine kinase inhibitor.

In some embodiments, the compound of the invention is used in combination with another anti-cancer therapy used for the treatment of uveal melanoma such as surgery, a MEK inhibitor, and/or a PKC inhibitor. For example, in some embodiments, the method further comprises performing surgery prior to, subsequent to, or at the same time as administration of the compound of the invention. In some embodiments, the method further comprises administration of a MEK inhibitor and/or a PKC inhibitor prior to, subsequent to, or at the same time as administration of the compound of the invention.

In some embodiments, the anticancer therapy and the compound of the invention are administered within 28 days of each other and each in an amount that together are effective to treat the subject.

In some embodiments, the subject or cancer has and/or has been identified as having a BRG1 loss of function mutation.

In some embodiments, the cancer is resistant to one or more chemotherapeutic or cytotoxic agents (e.g., the cancer has been determined to be resistant to chemotherapeutic or cytotoxic agents such as by genetic markers, or is likely to be resistant, to chemotherapeutic or cytotoxic agents such as a cancer that has failed to respond to a chemotherapeutic or cytotoxic agent). In some embodiments, the cancer has failed to respond to one or more chemotherapeutic or cytotoxic agents. In some embodiments, the cancer is resistant or has failed to respond to dacarbazine, temozolomide, cisplatin, treosulfan, fotemustine, IMCgp100, a CTLA-4 inhibitor (e.g., ipilimumab), a PD-1 inhibitor (e.g., Nivolumab or pembrolizumab), a PD-L1 inhibitor (e.g., atezolizumab, avelumab, or durvalumab), a mitogen-activated protein kinase (MEK) inhibitor (e.g., selumetinib, binimetinib, or tametinib), and/or a protein kinase C (PKC) inhibitor (e.g., sotrastaurin or IDE196).

In some embodiments, the cancer is resistant to or failed to respond to a previously administered therapeutic used for the treatment of uveal melanoma such as a MEK inhibitor or PKC inhibitor. For example, in some embodiments, the cancer is resistant to or failed to respond to a mitogen-activated protein kinase (MEK) inhibitor (e.g., selumetinib, binimetinib, or tametinib), and/or a protein kinase C (PKC) inhibitor (e.g., sotrastaurin or IDE196).

### Chemical Terms

The terminology employed herein is for the purpose of describing particular embodiments and is not intended to be limiting.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

The term “acyl,” as used herein, represents a H or an alkyl group that is attached to a parent molecular group through a carbonyl group, as defined herein, and is exemplified by formyl (i.e., a carboxaldehyde group), acetyl, trifluoroacetyl, propionyl, and butanoyl. Exemplary unsubstituted acyl groups include from 1 to 6, from 1 to 11, or from 1 to 21 carbons.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “alkynyl,” as used herein, alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6 carbon atoms, or 2 carbon atoms).

The term “amino,” as used herein, represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the invention can be an unsubstituted amino (i.e., —NH₂) or a substituted amino (i.e., —N(R^{N1})₂).

The term “aryl,” as used herein, refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

The term “arylalkyl,” as used herein, represents an alkyl group substituted with an aryl group.

Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

The term “azido,” as used herein, represents a —N₃ group.

The term “bridged polycycloalkyl,” as used herein, refers to a bridged polycyclic group of 5 to 20 carbons, containing from 1 to 3 bridges.

The term “cyano,” as used herein, represents a —CN group.

The term “carbocyclyl,” as used herein, refers to a non-aromatic C₃-C₁₂ monocyclic, bicyclic, or tricyclic structure in which the rings are formed by carbon atoms. Carbocyclyl structures include cycloalkyl groups and unsaturated carbocyclyl radicals.

The term “cycloalkyl,” as used herein, refers to a saturated, non-aromatic, and monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

The term “halo,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “heteroaryl,” as used herein, refers to a mono- or polycyclic radical of 5 to 12 atoms having at least one aromatic ring and containing 1, 2, or 3 ring atoms selected from nitrogen, oxygen, and sulfur, with the remaining ring atoms being carbon. One or two ring carbon atoms of the heteroaryl group may be replaced with a carbonyl group. Examples of heteroaryl groups are pyridyl, pyrazoyl, benzooxazolyl, benzoimidazolyl, benzothiazolyl, imidazolyl, oxaxolyl, and thiazolyl.

The term “heteroarylalkyl,” as used herein, represents an alkyl group substituted with a heteroaryl group. Exemplary unsubstituted heteroarylalkyl groups are from 7 to 30 carbons (e.g., from 7 to 16 or from 7 to 20 carbons, such as C₁-C₅ alkyl C₂-C₉ heteroaryl, C₁-C₁₀ alkyl C₂-C₆ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

The term “heterocyclyl,” as used herein, refers a mono- or polycyclic radical having 3 to 12 atoms having at least one ring containing 1, 2, 3, or 4 ring atoms selected from N, O or S, wherein no ring is aromatic. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl.

The term “heterocyclylalkyl,” as used herein, represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

The term “hydroxyalkyl,” as used herein, represents alkyl group substituted with an —OH group.

The term “hydroxyl,” as used herein, represents an —OH group.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, 0-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an —NO₂ group.

The term “thiol,” as used herein, represents an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Another exemplary substituent is oxo. For example, a carbonyl group is a carbon (e.g., alkyl carbon, alkenyl carbon, alkynyl carbon, heteroalkyl carbon, heteroalkenyl carbon, heteroalkynyl carbon, carbocyclyl carbon, etc.) substituted with oxo. Alternatively, sulfur may be substituted with one or two oxo groups (e.g., —SO— or —SO₂— within a substituted heteroalkyl, heteroalkenyl, heteroalkynyl, or heterocyclyl group). Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)). In some embodiments, alkyl, alkenyl, alkynyl, heteroalkyl, heteroalkenyl, and heteroalkynyl are optionally substituted with 1, 2, 3, 4, or 5 substituents independently selected from the group consisting of aryl (e.g., substituted and unsubstituted phenyl), carbocyclyl (e.g., substituted and unsubstituted cycloalkyl), halo (e.g., fluoro), hydroxyl, heteroaryl, heterocyclyl, amino (e.g., NH₂ or mono- or dialkyl amino), azido, cyano, nitro, thiol, and oxo. In some embodiments, the substituents are themselves unsubstituted.

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbents or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁵Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent. Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Definitions

In this application, unless otherwise clear from context, (i) the term “a” may be understood to mean “at least one”; (ii) the term “or” may be understood to mean “and/or”; and (iii) the terms “comprising” and “including” may be understood to encompass itemized components or steps whether presented by themselves or together with one or more additional components or steps.

As used herein, the terms “about” and “approximately” refer to a value that is within 10% above or below the value being described. For example, the term “about 5 nM” indicates a range of from 4.5 to 5.5 nM.

As used herein, the term “administration” refers to the administration of a composition (e.g., a compound or a preparation that includes a compound as described herein) to a subject or system.

Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intratumoral, intravenous, intraventricular, mucosal, nasal, oral, rectal, subcutaneous, sublingual, topical, tracheal (including by intratracheal instillation), transdermal, vaginal, and vitreal.

As used herein, the term “BAF complex” refers to the BRG1- or HRBM-associated factors complex in a human cell.

As used herein, the term “BAF complex-related disorder” refers to a disorder that is caused or affected by the level of activity of a BAF complex.

As used herein, the term “BRG1 loss of function mutation” refers to a mutation in BRG1 that leads to the protein having diminished activity (e.g., at least 1% reduction in BRG1 activity, for example 2%, 5%, 10%, 25%, 50%, or 100% reduction in BRG1 activity). Exemplary BRG1 loss of function mutations include, but are not limited to, a homozygous BRG1 mutation and a deletion at the C-terminus of BRG1.

As used herein, the term “BRG1 loss of function disorder” refers to a disorder (e.g., cancer) that exhibits a reduction in BRG1 activity (e.g., at least 1% reduction in BRG1 activity, for example 2%, 5%, 10%, 25%, 50%, or 100% reduction in BRG1 activity).

The term “cancer” refers to a condition caused by the proliferation of malignant neoplastic cells, such as tumors, neoplasms, carcinomas, sarcomas, leukemias, and lymphomas.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

By “determining the level” of a protein or RNA is meant the detection of a protein or an RNA, by methods known in the art, either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure RNA levels are known in the art and include, but are not limited to, quantitative polymerase chain reaction (qPCR) and Northern blot analyses.

By “decreasing the activity of a BAF complex” is meant decreasing the level of an activity related to a BAF complex, or a related downstream effect. A non-limiting example of decreasing an activity of a BAF complex is Sox2 activation. The activity level of a BAF complex may be measured using any method known in the art, e.g., the methods described in Kadoch et al. Cell, 2013, 153, 71-85, the methods of which are herein incorporated by reference.

As used herein, the term “degrader” refers to a small molecule compound including a degradation moiety, wherein the compound interacts with a protein (e.g., BRG1 and/or BRM) in a way which results in degradation of the protein, e.g., binding of the compound results in at least 5% reduction of the level of the protein, e.g., in a cell or subject.

As used herein, the term “degradation moiety” refers to a moiety whose binding results in degradation of a protein, e.g., BRG1 and/or BRM. In one example, the moiety binds to a protease or a ubiquitin ligase that metabolizes the protein, e.g., BRG1 and/or BRM.

By “modulating the activity of a BAF complex,” is meant altering the level of an activity related to a BAF complex (e.g., GBAF), or a related downstream effect. The activity level of a BAF complex may be measured using any method known in the art, e.g., the methods described in Kadoch et al, Cell 153:71-85 (2013), the methods of which are herein incorporated by reference.

By “reducing the activity of BRG1 and/or BRM,” is meant decreasing the level of an activity related to an BRG1 and/or BRM, or a related downstream effect. A non-limiting example of inhibition of an activity of BRG1 and/or BRM is decreasing the level of a BAF complex in a cell. The activity level of BRG1 and/or BRM may be measured using any method known in the art. In some embodiments, an agent which reduces the activity of BRG1 and/or BRM is a small molecule BRG1 and/or BRM degrader.

By “reducing the level of BRG1 and/or BRM,” is meant decreasing the level of BRG1 and/or BRM in a cell or subject. The level of BRG1 and/or BRM may be measured using any method known in the art.

By “level” is meant a level of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or mRNA in a sample.

As used herein, the term “inhibiting BRM” refers to blocking or reducing the level or activity of the ATPase catalytic binding domain or the bromodomain of the protein. BRM inhibition may be determined using methods known in the art, e.g., a BRM ATPase assay, a Nano DSF assay, or a BRM Luciferase cell assay.

The term “pharmaceutical composition,” as used herein, represents a composition containing a compound described herein formulated with a pharmaceutically acceptable excipient and appropriate for administration to a mammal, for example a human. Typically, a pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration.

As used herein, the term “pharmaceutically acceptable salt” means any pharmaceutically acceptable salt of a compound, for example, any compound of Formula I or II. Pharmaceutically acceptable salts of any of the compounds described herein may include those that are within the scope of sound medical judgment, suitable for use in contact with the tissues of humans and animals without undue toxicity, irritation, allergic response and are commensurate with a reasonable benefit/risk ratio.

Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

By a “reference” is meant any useful reference used to compare protein or RNA levels. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound of the invention; a sample from a subject that has been treated by a compound of the invention; or a sample of a purified protein or RNA (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder (e.g., cancer); a subject that has been treated with a compound of the invention. In preferred embodiments, the reference sample, standard, or level is matched to the sample subject sample by at least one of the following criteria: age, weight, sex, disease stage, and overall health. A standard curve of levels of a purified protein or RNA, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean therapeutic treatment or any measures whose object is to slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total); an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. Compounds of the invention may also be used to “prophylactically treat” or “prevent” a disorder, for example, in a subject at increased risk of developing the disorder.

The details of one or more embodiments of the invention are set forth in the description below. Other features, objects, and advantages of the invention will be apparent from the description and from the claims.

### DETAILED DESCRIPTION

The present disclosure features compounds useful for the inhibition of BRG1 and optionally BRM. These compounds may be used to modulate the activity of a BAF complex, for example, for the treatment of a BAF-related disorder, such as cancer (e.g., BRG1-loss of function disorders). Exemplary compounds described herein include compounds having a structure according to Formula I, or a pharmaceutically acceptable salt thereof.

### Formula I:

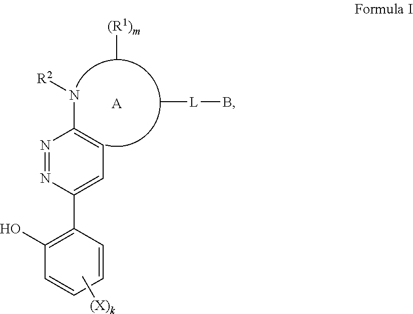 [0215] where
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, or optionally substituted C₃-C₈ cycloalkyl (e.g., each R¹ is, independently, halo or optionally substituted C₁-C₆ alkyl);
R² is H or optionally substituted C₁-C₆ alkyl;
each X is, independently, halo;
L is a linker; and
B is a degradation moiety.

In some embodiments, the compound has the structure of any one of compounds 1-310 in Table 1, or pharmaceutically acceptable salt thereof.

Other embodiments, as well as exemplary methods for the synthesis of production of these compounds, are described herein.

Compounds described herein may be prepared, e.g., using representative compounds shown in Table 2. The compounds in Table 2 include a binding moiety for targeting BRG1 and/or BRM.

TABLE 2

| # | Structure |
|---|---|
| B1 | 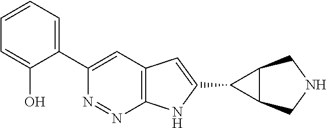 [SMILES: Oc1ccccc1-c1cc2cc([C@H]3[C@@H]4CNC[C@@H]43)[nH]c2nn1] |
| B2 | 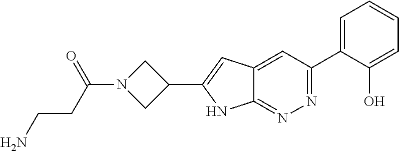 [SMILES: NCCC(=O)N1CC(c2cc3cc(-c4ccccc4O)nnc3[nH]2)C1] |
| B3 | 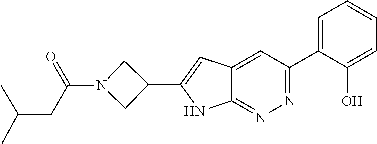 [SMILES: CC(C)CC(=O)N1CC(c2cc3cc(-c4ccccc4O)nnc3[nH]2)C1] |
| B4 | 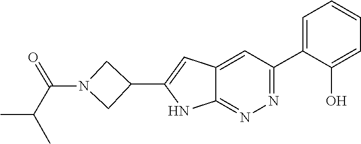 [SMILES: CC(C)C(=O)N1CC(c2cc3cc(-c4ccccc4O)nnc3[nH]2)C1] |
| B5 | 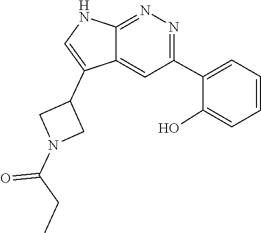 [SMILES: CCC(=O)N1CC(c2c[nH]c3nnc(-c4ccccc4O)cc23)C1] |
| B6 | 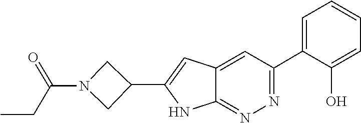 [SMILES: CCC(=O)N1CC(c2cc3cc(-c4ccccc4O)nnc3[nH]2)C1] |
| B7 | 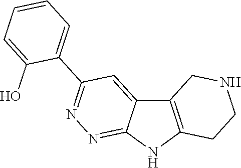 [SMILES: Oc1ccccc1-c1cc2c3c([nH]c2nn1)CCNC3] |
| B8 | 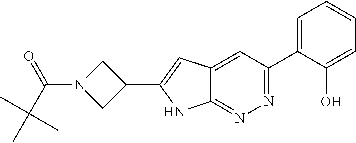 [SMILES: CC(C)(C)C(=O)N1CC(c2cc3cc(-c4ccccc4O)nnc3[nH]2)C1] |
| B9 | 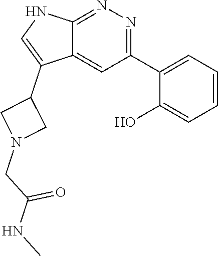 [SMILES: CNC(=O)CN1CC(c2c[nH]c3nnc(-c4ccccc4O)cc23)C1] |
| B10 | 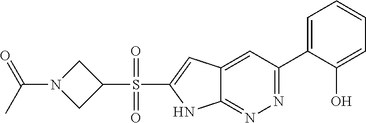 [SMILES: CC(=O)N1CC(S(=O)(=O)c2cc3cc(-c4ccccc4O)nnc3[nH]2)C1] |
| B11 | 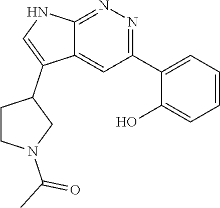 [SMILES: CC(=O)N1CCC(c2c[nH]c3nnc(-c4ccccc4O)cc23)C1] |
| B12 | 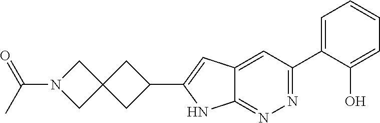 [SMILES: CC(=O)N1CC2(CC(c3cc4cc(-c5ccccc5O)nnc4[nH]3)C2)C1] |
| B13 | 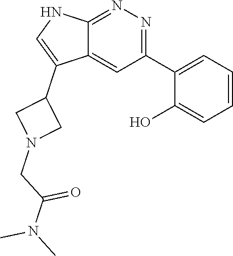 [SMILES: CN(C)C(=O)CN1CC(c2c[nH]c3nnc(-c4ccccc4O)cc23)C1] |
| B14 | 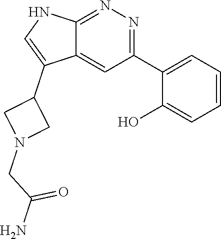 [SMILES: NC(=O)CN1CC(c2c[nH]c3nnc(-c4ccccc4O)cc23)C1] |
| B15 | 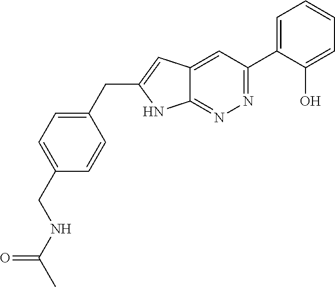 [SMILES: CC(=O)NCc1ccc(Cc2cc3cc(-c4ccccc4O)nnc3[nH]2)cc1] |
| B16 | 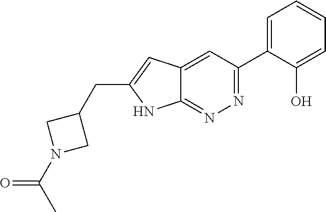 [SMILES: CC(=O)N1CC(Cc2cc3cc(-c4ccccc4O)nnc3[nH]2)C1] |
| B17 | 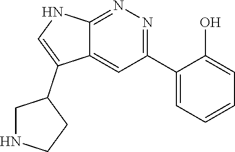 [SMILES: Oc1ccccc1-c1cc2c(C3CCNC3)c[nH]c2nn1] |
| B18 | 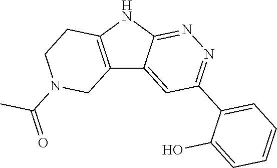 [SMILES: CC(=O)N1CCc2[nH]c3nnc(-c4ccccc4O)cc3c2C1] |
| B19 | 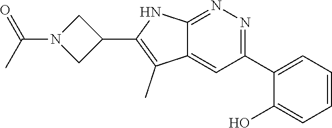 [SMILES: CC(=O)N1CC(c2[nH]c3nnc(-c4ccccc4O)cc3c2C)C1] |
| B20 | 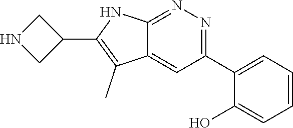 [SMILES: Cc1c(C2CNC2)[nH]c2nnc(-c3ccccc3O)cc12] |
| B21 | 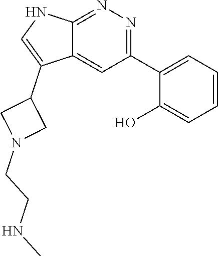 [SMILES: CNCCN1CC(c2c[nH]c3nnc(-c4ccccc4O)cc23)C1] |
| B22 | 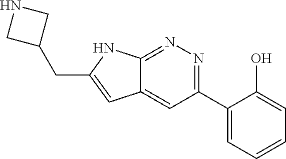 [SMILES: Oc1ccccc1-c1cc2cc(CC3CNC3)[nH]c2nn1] |
| B23 | 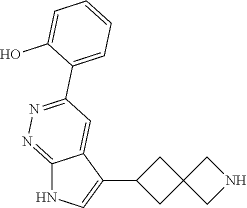 [SMILES: Oc1ccccc1-c1cc2c(C3CC4(CNC4)C3)c[nH]c2nn1] |
| B24 | 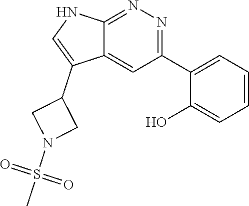 [SMILES: CS(=O)(=O)N1CC(c2c[nH]c3nnc(-c4ccccc4O)cc23)C1] |
| B25 | 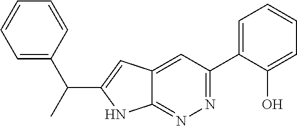 [SMILES: CC(c1ccccc1)c1cc2cc(-c3ccccc3O)nnc2[nH]1] |
| B26 | 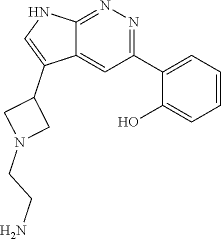 [SMILES: NCCN1CC(c2c[nH]c3nnc(-c4ccccc4O)cc23)C1] |
| B27 | 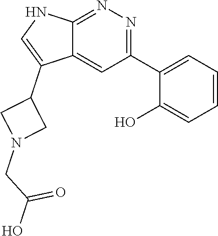 [SMILES: O=C(O)CN1CC(c2c[nH]c3nnc(-c4ccccc4O)cc23)C1] |
| B28 | 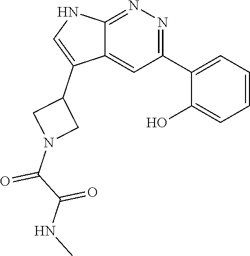 [SMILES: CNC(=O)C(=O)N1CC(c2c[nH]c3nnc(-c4ccccc4O)cc23)C1] |
| B29 | 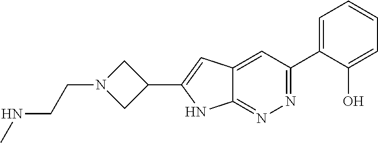 [SMILES: CNCCN1CC(c2cc3cc(-c4ccccc4O)nnc3[nH]2)C1] |
| B30 | 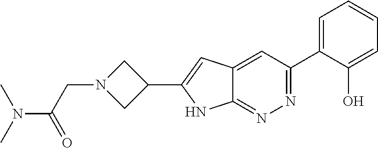 [SMILES: CN(C)C(=O)CN1CC(c2cc3cc(-c4ccccc4O)nnc3[nH]2)C1] |
| B31 | 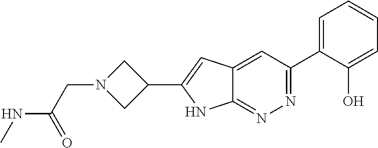 [SMILES: CNC(=O)CN1CC(c2cc3cc(-c4ccccc4O)nnc3[nH]2)C1] |
| B32 | 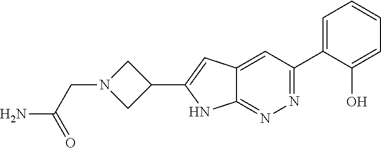 [SMILES: NC(=O)CN1CC(c2cc3cc(-c4ccccc4O)nnc3[nH]2)C1] |
| B33 | 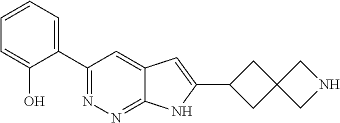 [SMILES: Oc1ccccc1-c1cc2cc(C3CC4(CNC4)C3)[nH]c2nn1] |
| B34 | 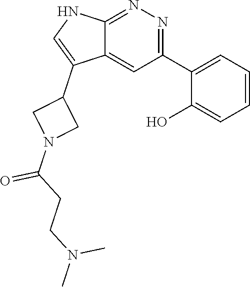 [SMILES: CN(C)CCC(=O)N1CC(c2c[nH]c3nnc(-c4ccccc4O)cc23)C1] |
| B35 | 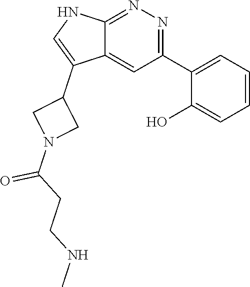 [SMILES: CNCCC(=O)N1CC(c2c[nH]c3nnc(-c4ccccc4O)cc23)C1] |
| B36 | 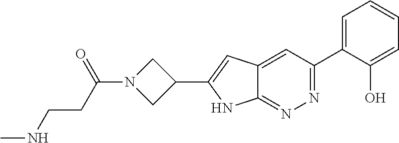 [SMILES: CNCCC(=O)N1CC(c2cc3cc(-c4ccccc4O)nnc3[nH]2)C1] |
| B37 | 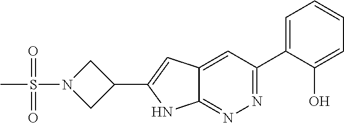 [SMILES: CS(=O)(=O)N1CC(c2cc3cc(-c4ccccc4O)nnc3[nH]2)C1] |
| B38 | 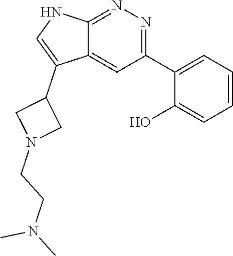 [SMILES: CN(C)CCN1CC(c2c[nH]c3nnc(-c4ccccc4O)cc23)C1] |
| B39 | 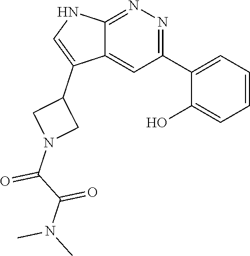 [SMILES: CN(C)C(=O)C(=O)N1CC(c2c[nH]c3nnc(-c4ccccc4O)cc23)C1] |
| B40 | 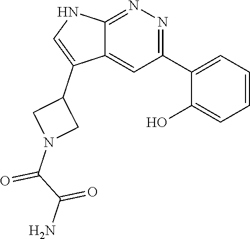 [SMILES: NC(=O)C(=O)N1CC(c2c[nH]c3nnc(-c4ccccc4O)cc23)C1] |
| B41 | 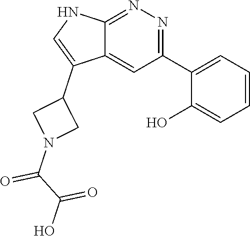 [SMILES: O=C(O)C(=O)N1CC(c2c[nH]c3nnc(-c4ccccc4O)cc23)C1] |
| B42 | 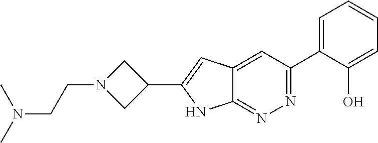 [SMILES: CN(C)CCN1CC(c2cc3cc(-c4ccccc4O)nnc3[nH]2)C1] |
| B43 | 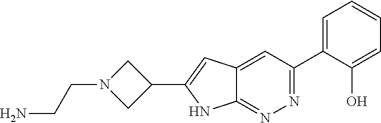 [SMILES: NCCN1CC(c2cc3cc(-c4ccccc4O)nnc3[nH]2)C1] |
| B44 | 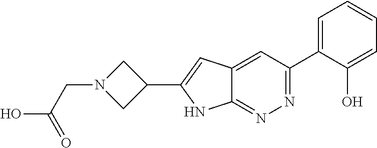 [SMILES: O=C(O)CN1CC(c2cc3cc(-c4ccccc4O)nnc3[nH]2)C1] |
| B45 | 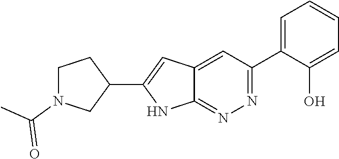 [SMILES: CC(=O)N1CCC(c2cc3cc(-c4ccccc4O)nnc3[nH]2)C1] |
| B46 | 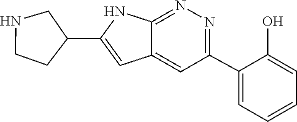 [SMILES: Oc1ccccc1-c1cc2cc(C3CCNC3)[nH]c2nn1] |
| B47 | 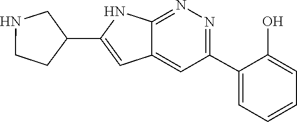 [SMILES: Oc1ccccc1-c1cc2cc(C3CCNC3)[nH]c2nn1] |
| B48 | 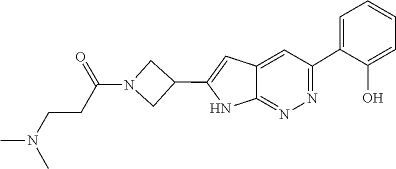 [SMILES: CN(C)CCC(=O)N1CC(c2cc3cc(-c4ccccc4O)nnc3[nH]2)C1] |
| B49 | 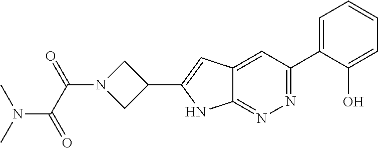 [SMILES: CN(C)C(=O)C(=O)N1CC(c2cc3cc(-c4ccccc4O)nnc3[nH]2)C1] |
| B50 | 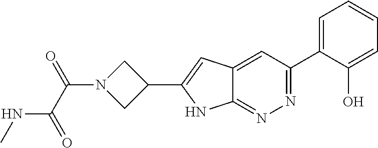 [SMILES: CNC(=O)C(=O)N1CC(c2cc3cc(-c4ccccc4O)nnc3[nH]2)C1] |
| B51 | 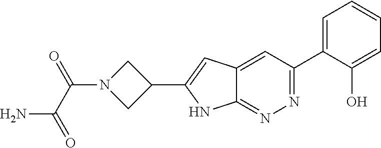 [SMILES: NC(=O)C(=O)N1CC(c2cc3cc(-c4ccccc4O)nnc3[nH]2)C1] |
| B52 | 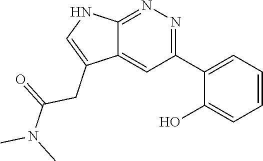 [SMILES: CN(C)C(=O)Cc1c[nH]c2nnc(-c3ccccc3O)cc12] |
| B53 | 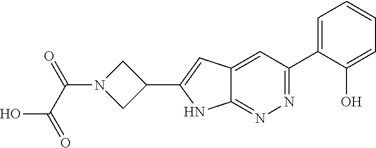 [SMILES: O=C(O)C(=O)N1CC(c2cc3cc(-c4ccccc4O)nnc3[nH]2)C1] |
| B54 | 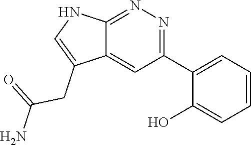 [SMILES: NC(=O)Cc1c[nH]c2nnc(-c3ccccc3O)cc12] |
| B55 | 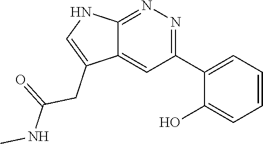 [SMILES: CNC(=O)Cc1c[nH]c2nnc(-c3ccccc3O)cc12] |
| B56 | 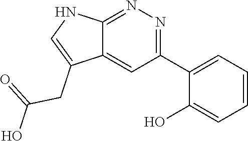 [SMILES: O=C(O)Cc1c[nH]c2nnc(-c3ccccc3O)cc12] |
| B57 | 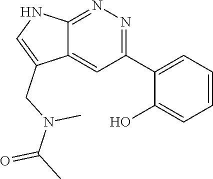 [SMILES: CC(=O)N(C)Cc1c[nH]c2nnc(-c3ccccc3O)cc12] |
| B58 | 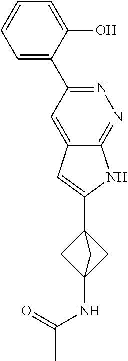 [SMILES: CC(=O)NC12CC(c3cc4cc(-c5ccccc5O)nnc4[nH]3)(C1)C2] |
| B59 | 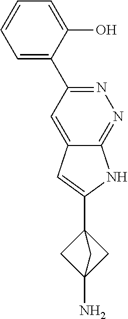 [SMILES: NC12CC(c3cc4cc(-c5ccccc5O)nnc4[nH]3)(C1)C2] |
| B60 | 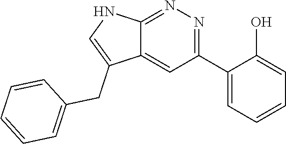 [SMILES: Oc1ccccc1-c1cc2c(Cc3ccccc3)c[nH]c2nn1] |
| B61 | 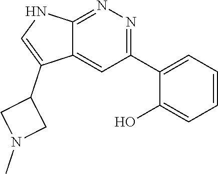 [SMILES: CN1CC(c2c[nH]c3nnc(-c4ccccc4O)cc23)C1] |
| B62 | 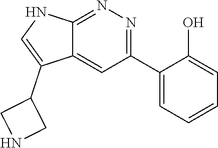 [SMILES: Oc1ccccc1-c1cc2c(C3CNC3)c[nH]c2nn1] |
| B63 | 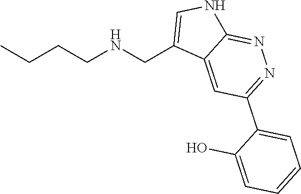 [SMILES: CCCCNCc1c[nH]c2nnc(-c3ccccc3O)cc12] |
| B64 | 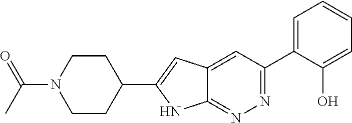 [SMILES: CC(=O)N1CCC(c2cc3cc(-c4ccccc4O)nnc3[nH]2)CC1] |
| B65 | 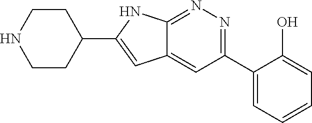 [SMILES: Oc1ccccc1-c1cc2cc(C3CCNCC3)[nH]c2nn1] |
| B66 | 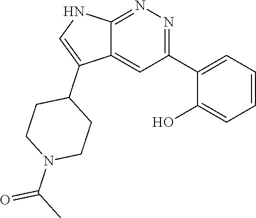 [SMILES: CC(=O)N1CCC(c2c[nH]c3nnc(-c4ccccc4O)cc23)CC1] |
| B67 | 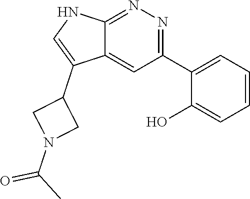 [SMILES: CC(=O)N1CC(c2c[nH]c3nnc(-c4ccccc4O)cc23)C1] |
| B68 | 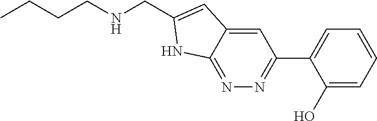 [SMILES: CCCCNCc1cc2cc(-c3ccccc3O)nnc2[nH]1] |
| B69 | 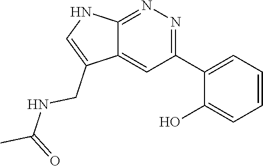 [SMILES: CC(=O)NCc1c[nH]c2nnc(-c3ccccc3O)cc12] |
| B70 | 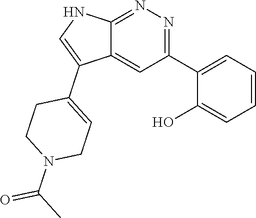 [SMILES: CC(=O)N1CC=C(c2c[nH]c3nnc(-c4ccccc4O)cc23)CC1] |
| B71 | 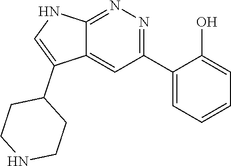 [SMILES: Oc1ccccc1-c1cc2c(C3CCNCC3)c[nH]c2nn1] |
| B72 | 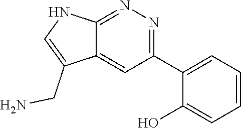 [SMILES: NCc1c[nH]c2nnc(-c3ccccc3O)cc12] |
| B73 | 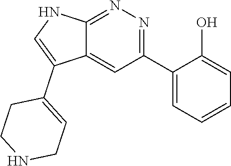 [SMILES: Oc1ccccc1-c1cc2c(C3=CCNCC3)c[nH]c2nn1] |
| B74 | 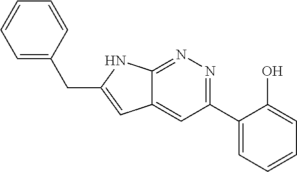 [SMILES: Oc1ccccc1-c1cc2cc(Cc3ccccc3)[nH]c2nn1] |
| B75 | 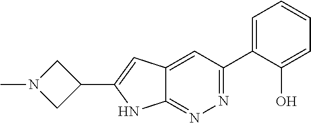 [SMILES: CN1CC(c2cc3cc(-c4ccccc4O)nnc3[nH]2)C1] |
| B76 | 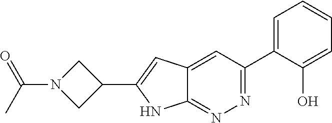 [SMILES: CC(=O)N1CC(c2cc3cc(-c4ccccc4O)nnc3[nH]2)C1] |
| B77 | 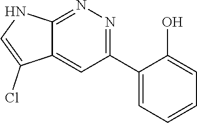 [SMILES: Oc1ccccc1-c1cc2c(Cl)c[nH]c2nn1] |
| B78 | 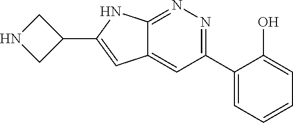 [SMILES: Oc1ccccc1-c1cc2cc(C3CNC3)[nH]c2nn1] |
| B79 | 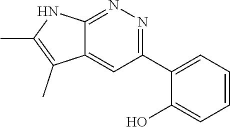 [SMILES: Cc1[nH]c2nnc(-c3ccccc3O)cc2c1C] |
| B80 | 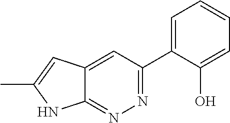 [SMILES: Cc1cc2cc(-c3ccccc3O)nnc2[nH]1] |
| B81 | 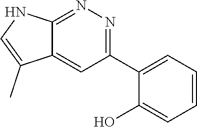 [SMILES: Cc1c[nH]c2nnc(-c3ccccc3O)cc12] |
| B82 | 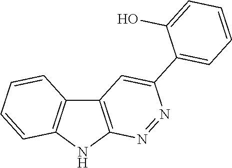 [SMILES: Oc1ccccc1-c1cc2c(nn1)[nH]c1ccccc12] |
| B83 | 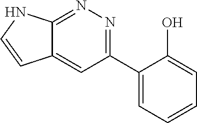 [SMILES: Oc1ccccc1-c1cc2cc[nH]c2nn1] |
| B84 | 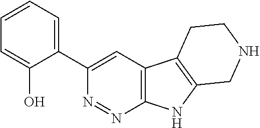 [SMILES: Oc1ccccc1-c1cc2c3c([nH]c2nn1)CNCC3] |
| B85 | 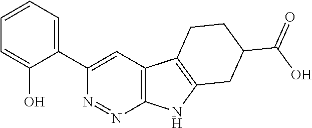 [SMILES: O=C(O)C1CCc2c([nH]c3nnc(-c4ccccc4O)cc23)C1] |
| B86 | 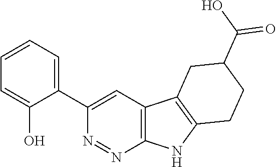 [SMILES: O=C(O)C1CCc2[nH]c3nnc(-c4ccccc4O)cc3c2C1] |

### Pharmaceutical Uses

The compounds described herein are useful in the methods of the invention and, while not bound by theory, are believed to exert their ability to modulate the level, status, and/or activity of a BAF complex, i.e., by inhibiting the activity of the BRG1 and/or BRM proteins within the BAF complex in a mammal. BAF complex-related disorders include, but are not limited to, BRG1 loss of function mutation-related disorders.

An aspect of the present invention relates to methods of treating disorders related to BRG1 loss of function mutations such as cancer (e.g., non-small cell lung cancer, colorectal cancer, bladder cancer, cancer of unknown primary, glioma, breast cancer, melanoma, non-melanoma skin cancer, endometrial cancer, or penile cancer) in a subject in need thereof. In some embodiments, the compound is administered in an amount and for a time effective to result in one or more (e.g., two or more, three or more, four or more) of: (a) reduced tumor size, (b) reduced rate of tumor growth, (c) increased tumor cell death (d) reduced tumor progression, (e) reduced number of metastases, (f) reduced rate of metastasis, (g) decreased tumor recurrence (h) increased survival of subject, (i) increased progression free survival of subject.

Treating cancer can result in a reduction in size or volume of a tumor. For example, after treatment, tumor size is reduced by 5% or greater (e.g., 10%, 20%, 30%, 40%, 50%, 60%, 70%, 80%, 90%, or greater) relative to its size prior to treatment. Size of a tumor may be measured by any reproducible means of measurement. For example, the size of a tumor may be measured as a diameter of the tumor.

Treating cancer may further result in a decrease in number of tumors. For example, after treatment, tumor number is reduced by 5% or greater (e.g., 10%, 20%, 30%, 40%, 50%, 60%, 70%, 80%, 90%, or greater) relative to number prior to treatment. Number of tumors may be measured by any reproducible means of measurement, e.g., the number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic nodules may be measured by any reproducible means of measurement. For example, the number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, or 120 days). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with a pharmaceutically acceptable salt of the invention.

Treating cancer can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a pharmaceutically acceptable salt of the invention. A decrease in the mortality rate of a population may also be measured, for example, by calculating for a population the average number of disease-related deaths per unit time following completion of a first round of treatment with a pharmaceutically acceptable salt of the invention.

Exemplary cancers that may be treated by the invention include, but are not limited to, non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer and penile cancer.

### Combination Formulations and Uses Thereof

The compounds of the invention can be combined with one or more therapeutic agents. In particular, the therapeutic agent can be one that treats or prophylactically treats any cancer described herein.

### Combination Therapies

A compound of the invention can be used alone or in combination with an additional therapeutic agent, e.g., other agents that treat cancer or symptoms associated therewith, or in combination with other types of treatment to treat cancer. In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2005). In this case, dosages of the compounds when combined should provide a therapeutic effect.

In some embodiments, the second therapeutic agent is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, Adriamycin® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), ABraxane®, cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art and described in, for example, Saltz et al. (1999) Proc ASCO 18:233a and Douillard et al. (2000) Lancet 355:1041-7.

In some embodiments, the second therapeutic agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (Avastin®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response or antagonizes an antigen important for cancer. Such agents include Rituxan (Rituximab); Zenapax (Daclizumab); Simulect (Basiliximab); Synagis (Palivizumab); Remicade (Infliximab); Herceptin (Trastuzumab); Mylotarg (Gemtuzumab ozogamicin); Campath (Alemtuzumab); Zevalin (Ibritumomab tiuxetan); Humira (Adalimumab); Xolair (Omalizumab); Bexxar (Tositumomab-I-131); Raptiva (Efalizumab); Erbitux (Cetuximab); Avastin (Bevacizumab); Tysabri (Natalizumab); Actemra (Tocilizumab); Vectibix (Panitumumab); Lucentis (Ranibizumab); Soliris (Eculizumab); Cimzia (Certolizumab pegol); Simponi (Golimumab); Ilaris (Canakinumab); Stelara (Ustekinumab); Arzerra (Ofatumumab); Prolia (Denosumab); Numax (Motavizumab); ABThrax (Raxibacumab); Benlysta (Belimumab); Yervoy (Ipilimumab); Adcetris (Brentuximab Vedotin); Perjeta (Pertuzumab); Kadcyla (Ado-trastuzumab emtansine); and Gazyva (Obinutuzumab). Also included are antibody-drug conjugates.

The second agent may be a therapeutic agent which is a non-drug treatment. For example, the second therapeutic agent is radiation therapy, cryotherapy, hyperthermia and/or surgical excision of tumor tissue.

The second agent may be a checkpoint inhibitor. In one embodiment, the inhibitor of checkpoint is an inhibitory antibody (e.g., a monospecific antibody such as a monoclonal antibody). The antibody may be, e.g., humanized or fully human. In some embodiments, the inhibitor of checkpoint is a fusion protein, e.g., an Fc-receptor fusion protein. In some embodiments, the inhibitor of checkpoint is an agent, such as an antibody, that interacts with a checkpoint protein. In some embodiments, the inhibitor of checkpoint is an agent, such as an antibody, that interacts with the ligand of a checkpoint protein. In some embodiments, the inhibitor of checkpoint is an inhibitor (e.g., an inhibitory antibody or small molecule inhibitor) of CTLA-4 (e.g., an anti-CTLA4 antibody such as ipilimumab/Yervoy or tremelimumab). In some embodiments, the inhibitor of checkpoint is an inhibitor (e.g., an inhibitory antibody or small molecule inhibitor) of PD-1 (e.g., nivolumab/Opdivo®; pembrolizumab/Keytruda®; pidilizumab/CT-011). In some embodiments, the inhibitor of checkpoint is an inhibitor (e.g., an inhibitory antibody or small molecule inhibitor) of PDL1 (e.g., MPDL3280A/RG7446; MEDI4736; MSB0010718C; BMS 936559). In some embodiments, the inhibitor of checkpoint is an inhibitor (e.g., an inhibitory antibody or Fc fusion or small molecule inhibitor) of PDL2 (e.g., a PDL2/Ig fusion protein such as AMP 224). In some embodiments, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof.

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### Pharmaceutical Compositions

The compounds of the invention are preferably formulated into pharmaceutical compositions for administration to a mammal, preferably, a human, in a biologically compatible form suitable for administration in vivo. Accordingly, in an aspect, the present invention provides a pharmaceutical composition comprising a compound of the invention in admixture with a suitable diluent, carrier, or excipient.

The compounds of the invention may be used in the form of the free base, in the form of salts, solvates, and as prodrugs. All forms are within the scope of the invention. In accordance with the methods of the invention, the described compounds or salts, solvates, or prodrugs thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, ortransdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

A compound of the invention may be orally administered, for example, with an inert diluent or with an assimilable edible carrier, or it may be enclosed in hard- or soft-shell gelatin capsules, or it may be compressed into tablets, or it may be incorporated directly with the food of the diet. For oral therapeutic administration, a compound of the invention may be incorporated with an excipient and used in the form of ingestible tablets, buccal tablets, troches, capsules, elixirs, suspensions, syrups, and wafers. A compound of the invention may also be administered parenterally. Solutions of a compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003, 20th ed.) and in The United States Pharmacopeia: The National Formulary (USP 24 NF19), published in 1999. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer. Compositions suitable for buccal or sublingual administration include tablets, lozenges, and pastilles, where the active ingredient is formulated with a carrier, such as sugar, acacia, tragacanth, gelatin, and glycerine. Compositions for rectal administration are conveniently in the form of suppositories containing a conventional suppository base, such as cocoa butter. A compound described herein may be administered intratumorally, for example, as an intratumoral injection. Intratumoral injection is injection directly into the tumor vasculature and is specifically contemplated for discrete, solid, accessible tumors. Local, regional, or systemic administration also may be appropriate. A compound described herein may advantageously be contacted by administering an injection or multiple injections to the tumor, spaced for example, at approximately, 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature.

The compounds of the invention may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

### Dosages

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, satisfactory results may be obtained when the compounds of the invention are administered to a human at a daily dosage of, for example, between 0.05 mg and 3000 mg (measured as the solid form). Dose ranges include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

Alternatively, the dosage amount can be calculated using the body weight of the patient. For example, the dose of a compound, or pharmaceutical composition thereof, administered to a patient may range from 0.1-100 mg/kg (e.g., 0.25-25 mg/kg). In exemplary, non-limiting embodiments, the dose may range from 0.5-5.0 mg/kg (e.g., 0.5, 1.0, 1.5, 2.0, 2.5, 3.0, 3.5, 4.0, 4.5, or 5.0 mg/kg) or from 5.0-20 mg/kg (e.g., 5.5, 6.0, 6.5, 7.0, 7.5, 8.0, 8.5, 9.0, 9.5, 10, 11, 12, 13, 14, 15, 16, 17, 18, 19, or 20 mg/kg).

### EXAMPLES

The following abbreviations are used throughout the Examples below.
Ac acetyl
ACN or MeCN acetonitrile
AcOH acetic acid
Ac₂O acetic anhydride
aq. aqueous
Boc tert-butoxycarbonyl
Bu or n-Bu butyl
CDI 1,1′-carbonyldiimidazole
DCE or 1,2-DCE 1,2-dichloroethane
DCM dichloromethane
DIAD diisopropyl azodicarboxylate
DIPEA or DIEA N.N-diisopropylethylamine
DMAP 4-(dimethylamino)pyridine
DMB 2,4-dimethoxybenzyl
DME 1,2-dimethoxyethane
DMF N.N-dimethylformamide
DMSO dimethyl sulfoxide
EA or EtOAc ethyl acetate
EDCI N-(3-dimethylaminopropyl)-N′-ethylcarbodiimide hydrochloride
equiv equivalents
Et₃N or TEA triethylamine
EtOH ethyl alcohol
FA formic acid
h or hr hour
HATU 1-[bis(dimethylamino)methylene]-1H-1,2,3-triazolo[4,5-b]pyridinium 3-oxid hexafluorophosphate
HOAt 1-hydroxy-7-azabenzotriazole
HOBt or HOBT 1-hydroxybenzotriazole hydrate
iPr Isopropyl
MeOH methyl alcohol
Me₄t-BuXphos ditert-butyl-[2,3,4,5-tetramethyl-6-(2,4,6-triisopropylphenyl)phenyl]phosphane
min minute
MTBE tert-butyl methyl ether
n-BuLi n-butylithium
NMP 1-methyl-2-pyrrolidinone
OAc acetate
Pd/C palladium on carbon
PDC pyridinium dichromate
PdCl₂(dtbpf) or Pd(dtbpf)Cl₂ dichloro[1,1′-bis(di-t-butylphosphino)ferrocene]palladium(II)
PdCl₂(dppf) or Pd(dppf)Cl₂ [1,1′-bis(diphenylphosphino)ferrocene]dichloropalladium(II)
Pd₂(dba)₃ tris(dibenzylideneacetone)dipalladium(0)
Pd(PPh₃)₄ tetrakis(triphenylphosphine)palladium(0
Pd(PPh₃)₂Cl₂ dichlorobis(triphenylphosphine)palladium(II)
PE petroleum ether
PPh₃ triphenylphosphine
Pr n-propyl
Py pyridine
rac racemic
Rf retention factor
r.t. or rt room temperature
sat. saturated
SFC supercritical fluid chromatography
t-Bu tert-butyl
tBuXphos-Pd-G3 or
tBuXphos Pd G₃ or
t-BuXphos-Pd (gen 3) [2-(2-aminophenyl)phenyl]-methylsulfonyloxypalladium;ditert-butyl-[2-(2,4,6-triisopropylphenyl)phenyl]phosphane
TFA trifluoroacetic acid
Tf₂O trifluoromethanesulfonic anhydride
THE tetrahydrofuran
TLC thin layer chromatography
Xantphos-Pd-G3 [2-(2-aminophenyl)phenyl]-methylsulfonyloxy-palladium;(5-diphenylphosphanyl-9,9-dimethyl-xanthen-4-yl)-diphenyl-phosphane
XPhos Pd G3 (2-Dicyclohexylphosphino-2′,4′,6′-triisopropyl-1,1′-biphenyl)[2-(2′-amino-1,1′-biphenyl)]palladium(II) methanesulfonate

### Example 1. Preparation of Intermediates

### Preparation of 2-(6-(azetidin-3-yl)-7H-pyrrolo[2,3-c]pyridazin-3-yl)phenol (I-1)

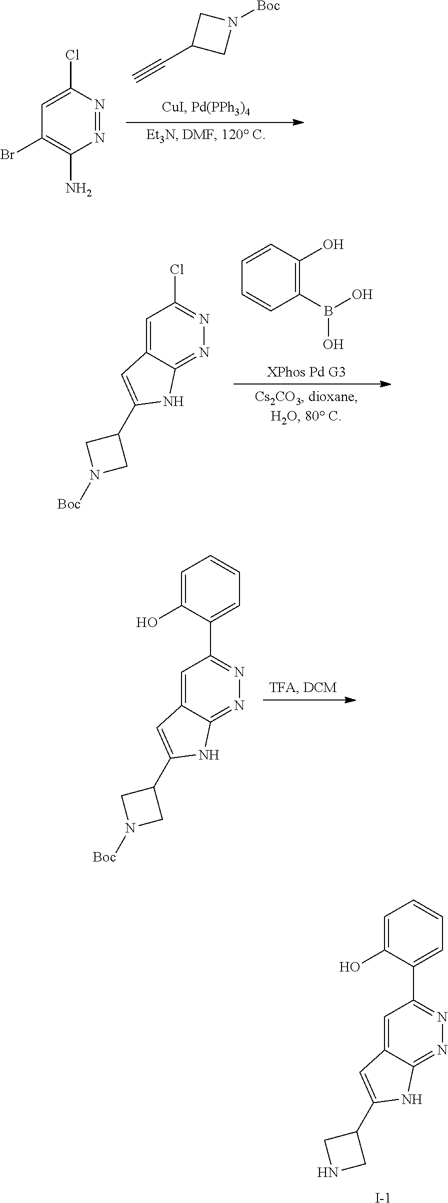

### Step 1: Preparation of tert-butyl 3-[3-chloro-7H-pyrrolo[2,3-c]pyridazin-6-yl]azetidine-1-carboxylate

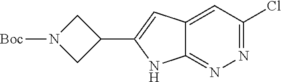 [SMILES: CC(C)(C)OC(=O)N1CC(c2cc3cc(Cl)nnc3[nH]2)C1]

To a stirred solution of 4-bromo-6-chloropyridazin-3-amine (345 mg, 1.66 mmol) and tert-butyl 3-ethynylazetidine-1-carboxylate (300 mg, 1.66 mmol) in DMF (10.0 mL) was added Pd(PPh₃)₂Cl₂ (383 mg, 0.331 mmol), CuI (63.1 mg, 0.331 mmol) and Et₃N (1.68 g, 16.6 mmol) at room temperature. The resulting mixture was stirred for 16 h at 120° C. under a nitrogen atmosphere. The residue was purified by reverse phase C18 flash chromatography (Water:ACN:FA) to afford tert-butyl 3-[3-chloro-7H-pyrrolo[2,3-c]pyridazin-6-yl]azetidine-1-carboxylate (175 mg, 34.2%) as a black solid. LCMS (ESI) m/z: [M+H]⁺=309.

### Step 2: Preparation of tert-butyl 3-[3-(2-hydroxyphenyl)-7H-pyrrolo[2,3-c]pyridazin-6-yl]azetidine-1-carboxylate

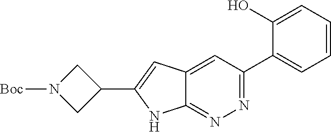 [SMILES: CC(C)(C)OC(=O)N1CC(c2cc3cc(-c4ccccc4O)nnc3[nH]2)C1]

To a stirred solution of tert-butyl 3-[3-chloro-7H-pyrrolo[2,3-c]pyridazin-6-yl]azetidine-1-carboxylate (175 mg, 0.567 mmol) and 2-hydroxyphenylboronic acid (156 mg, 1.13 mmol) in dioxane (5.00 mL) and H₂O (1.00 mL) were added XPhos Pd G3 (48.0 mg, 0.057 mmol) and Cs₂CO₃ (554 mg, 1.70 mmol) at room temperature under a nitrogen atmosphere. The resulting mixture was stirred for 4 h at 100° C. under an atmosphere of dry nitrogen. The reaction was quenched by the addition of water at room temperature. The resulting mixture was extracted three times with EtOAc. The combined organic layers were washed with brine, and dried over anhydrous Na₂SO₄. After filtration, the filtrate was concentrated under reduced pressure. The residue was purified by reverse phase C18 flash chromatography (Water:ACN:FA) to afford tert-butyl 3-[3-(2-hydroxyphenyl)-7H-pyrrolo[2,3-c]pyridazin-6-yl]azetidine-1-carboxylate (138 mg, 66.5%) as a light yellow solid. LCMS (ESI) m/z: [M+H]⁺=367.

### Step 3: Preparation of 2-[6-(azetidin-3-yl)-7H-pyrrolo[2,3-c]pyridazin-3-yl]phenol (I-1)

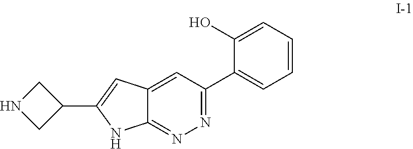 [SMILES: Oc1ccccc1-c1cc2cc(C3CNC3)[nH]c2nn1]

To a stirred solution of tert-butyl 3-[3-(2-hydroxyphenyl)-7H-pyrrolo[2,3-c]pyridazin-6-yl]azetidine-1-carboxylate (30.0 mg, 0.082 mmol) in DCM (2.00 mL) was added TFA (1.00 mL) dropwise at room temperature. The resulting mixture was stirred for 1 h at room temperature. The resulting mixture was concentrated under reduced pressure. The crude product was purified by Prep-HPLC (Water:ACN:FA) to afford I-1 (11.0 mg, 50.5%) as an off-white solid. ¹H NMR (400 MHz, DMSO-d₆) δ 13.94 (s, 1H), 9.11 (s, 1H), 8.66 (s, 1H), 8.06 (dd, J=8.0, 1.6 Hz, 1H), 7.31 (ddd, J=8.5, 7.2, 1.6 Hz, 1H), 6.97 (t, J=7.8 Hz, 2H), 6.76 (s, 1H), 4.45-4.23 (m, 5H). LCMS (ESI) m/z: [M+H]⁺=267.20.

The following intermediates in Table 3 were prepared in a similar manner as described in the preparation of intermediate I-1 from 4-bromo-6-chloropyridazin-3-amine and the appropriate alkyne.

TABLE 3

| | Intermediate | | LCMS |
|---|---|---|---|
| Structure | No. | Name | (ESI) m/z |
| 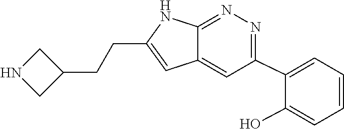 [SMILES: Oc1ccccc1-c1cc2cc(CCC3CNC3)[nH]c2nn1] | I-55 | (2-{6-[2-(azetidin-3-yl)ethyl]- 7H-pyrrolo[2,3-c]pyridazin-3- yl}phenol | 395 |
| 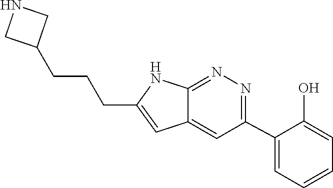 [SMILES: Oc1ccccc1-c1cc2cc(CCCC3CNC3)[nH]c2nn1] | I-56 | 2-{6-[3-(azetidin-3-yl)propyl]- 7H-pyrrolo[2,3-c]pyridazin-3- yl}phenol | 309 |
| 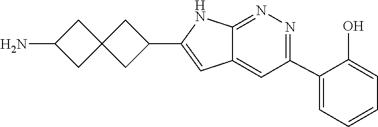 [SMILES: NC1CC2(C1)CC(c1cc3cc(-c4ccccc4O)nnc3[nH]1)C2] | I-57 | 2-(6-(6-aminospiro[3.3]heptan- 2-yl)-7H-pyrrolo[2,3- c]pyridazin-3-yl)phenol | 321 |
| 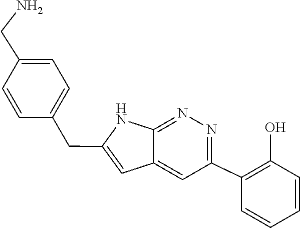 [SMILES: NCc1ccc(Cc2cc3cc(-c4ccccc4O)nnc3[nH]2)cc1] | I-58 | 2-(6-[[4- (aminomethyl)phenyl]methyl]- 7H-pyrrolo[2,3-c]pyridazin-3- yl)phenol | 331 |
| 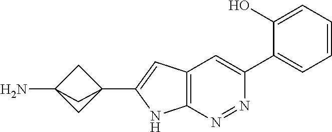 [SMILES: NC12CC(c3cc4cc(-c5ccccc5O)nnc4[nH]3)(C1)C2] | I-85 | 2-(6-(3- aminobicyclo[1.1.1]pentan-1- yl)-7H-pyrrolo[2,3-c]pyridazin- 3-yl)phenol | 293 |
| 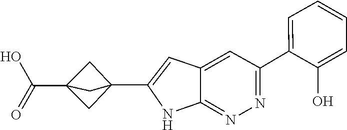 [SMILES: O=C(O)C12CC(c3cc4cc(-c5ccccc5O)nnc4[nH]3)(C1)C2] | I-86 | 3-(3-(2-hydroxyphenyl)-7H- pyrrolo[2,3-c]pyridazin-6- yl)bicyclo[1.1.1]pentane-1- carboxylic acide | 322 |

### Preparation of 2-[5-(azetidin-3-yl)-7H-pyrrolo[2,3-c]pyridazin-3-yl]phenol (I-2)

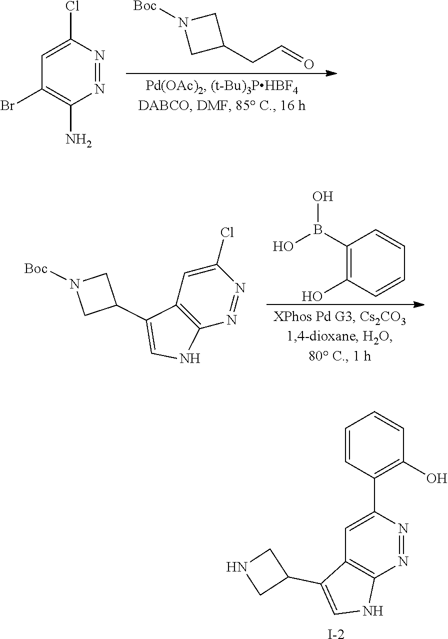

### Step 1: Preparation of tert-butyl 3-[3-chloro-7H-pyrrolo[2,3-c]pyridazin-5-yl]azetidine-1-carboxylate

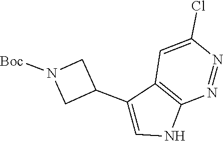 [SMILES: CC(C)(C)OC(=O)N1CC(c2c[nH]c3nnc(Cl)cc23)C1]

To a stirred solution of 4-bromo-6-chloropyridazin-3-amine (300 mg, 1.44 mmol) and tert-butyl 3-(2-oxoethyl)azetidine-1-carboxylate (287 mg, 1.44 mmol) in DMF (5.00 mL) was added Pd(OAc)₂ (32.3 mg, 0.144 mmol), (t-Bu)₃P·HBF₄ (41.8 mg, 0.144 mmol) and 1,4-diazabicyclo[2,2,2]octane (484 mg, 4.32 mmol) at room temperature. Following stirring for 16 h at 85° C. under a nitrogen atmosphere, the mixture was allowed to cool down to room temperature. The reaction mixture was filtered through a short pad of Celite and concentrated in vacuo. The residue was purified by reverse phase C18 flash chromatography (Water:ACN:FA) to afford tert-butyl 3-[3-chloro-7H-pyrrolo[2,3-c]pyridazin-5-yl]azetidine-1-carboxylate (115 mg, 25.9%) as a brown solid.

### Step 2: Preparation of 2-[5-(azetidin-3-yl)-7H-pyrrolo[2,3-c]pyridazin-3-yl]phenol (I-2)

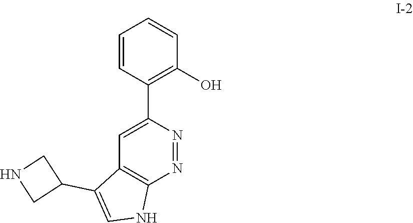 [SMILES: Oc1ccccc1-c1cc2c(C3CNC3)c[nH]c2nn1]

To a stirred solution of tert-butyl 3-[3-chloro-7H-pyrrolo[2,3-c]pyridazin-5-yl]azetidine-1-carboxylate (115 mg, 0.372 mmol) and 2-hydroxyphenylboronic acid (154 mg, 1.12 mmol) in 1,4-dioxane (8.00 mL) and H₂O (2.00 mL) was added XPhos Pd G3 (62.6 mg, 0.074 mmol) and Cs₂CO₃ (364 mg, 1.12 mmol) at room temperature. After stirring for 1 h at 80° C. under a nitrogen atmosphere, the mixture was allowed to cool down to room temperature. The reaction mixture was filtered through a short pad of Celite and concentrated in vacuo. The residue was purified by Prep-HPLC (Water:ACN:FA) to afford I-2 (5.7 mg, 4.19%) as a yellow solid. ¹H NMR (400 MHz, DMSO-d₆) δ 14.03 (s, 1H), 8.87 (s, 1H), 8.45 (s, 1H), 8.27-8.05 (m, 2H), 7.31 (t, J=7.8 Hz, 1H), 7.07-6.92 (m, 2H), 4.39-4.30 (m, 1H), 4.26-4.10 (m, 4H). LCMS (ESI) m/z: [M+H]⁺=267.05.

### Preparation of 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (I-10)

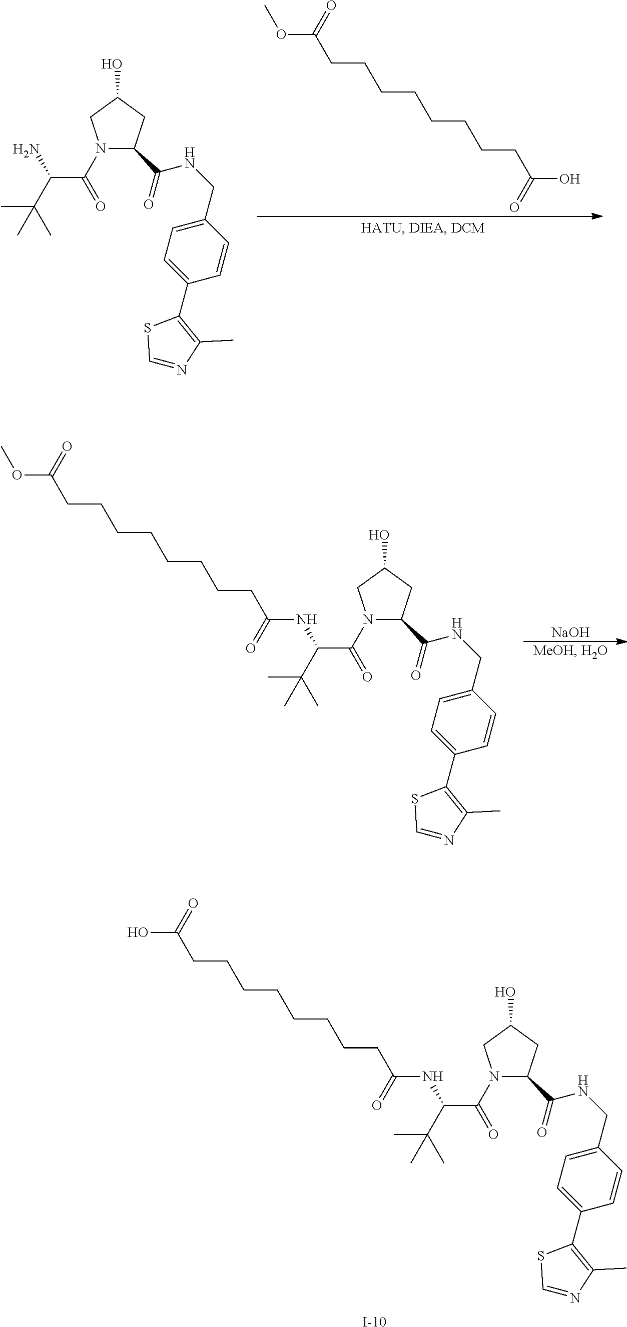

### Step 1: Preparation of methyl 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoate

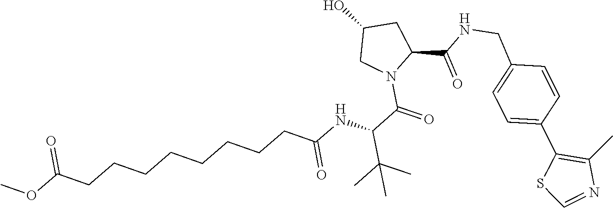

To a solution of 10-methoxy-10-oxo-decanoic acid (136 mg, 0.627 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (300 mg, 0.697 mmol) in DCM (3 mL) was added HATU (265 mg, 0.697 mmol) and DIEA (485 μL, 2.79 mmol). After addition, the mixture was stirred at 25° C. for 2 h. The reaction mixture was quenched by water and then extracted three times with DCM. The combined organic layers were washed twice with brine, and dried over Na₂SO₄, filtered and concentrated under reduced pressure to give a residue. The residue was purified by reversed phase chromatography to afford methyl 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoate (175 mg, 39.9% yield) as a yellow oil. LCMS (ESI) m/z: [M+H]⁺=629.5.

### Step 2: Preparation of 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (I-10)

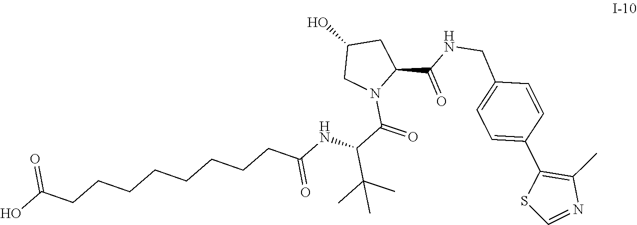 [SMILES: Cc1ncsc1-c1ccc(CNC(=O)[C@@H]2C[C@@H](O)CN2C(=O)[C@@H](NC(=O)CCCCCCCCC(=O)O)C(C)(C)C)cc1]

To a solution of methyl 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoate (170 mg, 0.270 mmol) in MeOH (1.5 mL) and H₂O (0.5 mL) was added NaOH (21.6 mg, 0.541 mmol) at 25° C. The mixture was stirred at this temperature for 12 h. The reaction mixture was adjusted neutral pH with by hydrochloric acid (2 M). The residue was purified by reversed phase chromatography to afford 10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (115 mg, 69.5% yield) as a white solid. ¹H NMR (400 MHz, DMSO-d₆) δ 12.25-11.73 (m, 1H), 8.99 (s, 1H), 8.57-8.55 (m, 1H), 7.84 (d, J=9.2 Hz, 1H), 7.45-7.36 (m, 4H), 5.24-5.05 (m, 1H), 4.56-4.21 (m, 5H), 3.71-3.61 (m, 2H), 2.45 (s, 3H), 2.28-1.89 (m, 7H), 1.54-1.41 (m, 4H), 1.24 (s, 8H), 0.97-0.91 (m, 9H). LCMS (ESI) m/z: [M+H]⁺=615.5.

The following intermediates in Table 4 were prepared in a similar manner as described in the preparation of intermediate I-10 from (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide and the appropriate carboxylic acid.

TABLE 4

| | | | LCMS |
|---|---|---|---|
| | Intermediate | | (ESI) |
| Structure | No. | Name | m/z |
| 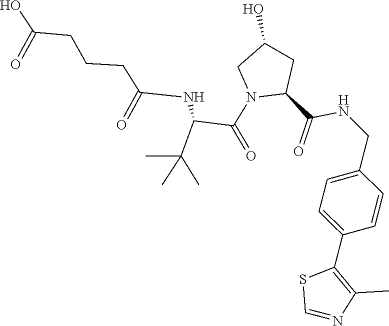 [SMILES: Cc1ncsc1-c1ccc(CNC(=O)[C@@H]2C[C@@H](O)CN2C(=O)[C@@H](NC(=O)CCCC(=O)O)C(C)(C)C)cc1] | I-46 | 5-[[(1S)-1- [(2S,4R)-4-hydroxy- 2-[4-(4-methylthiazol-5- yl)phenyl]methylcarbamoyl] pyrrolidine-1-carbonyl]-2,2- dimethyl-propyl] amino]-5-oxo- pentanoic acid | 567.4 |
| 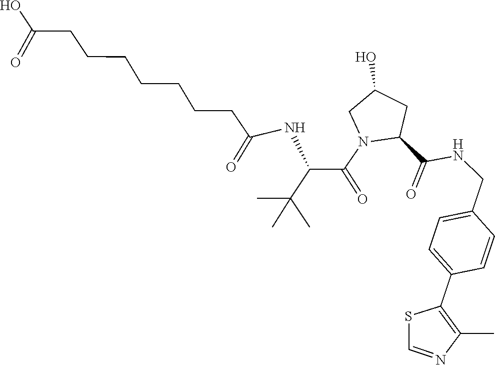 [SMILES: Cc1ncsc1-c1ccc(CNC(=O)[C@@H]2C[C@@H](O)CN2C(=O)[C@@H](NC(=O)CCCCCCCC(=O)O)C(C)(C)C)cc1] | I-47 | 9-(((S)-1-((2S,4R)- 4-hydroxy- 2-((4-(4-methylthiazol-5- yl)benzyl)carbamoyl) pyrrolidin- 1-yl)-3,3-dimethyl- 1-oxobutan- 2-yl)amino)-9-oxononanoic acid | 601.4 |
| 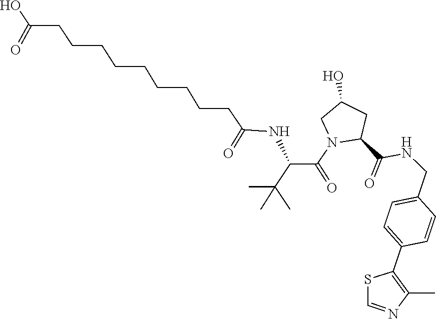 [SMILES: Cc1ncsc1-c1ccc(CNC(=O)[C@@H]2C[C@@H](O)CN2C(=O)[C@@H](NC(=O)CCCCCCCCCC(=O)O)C(C)(C)C)cc1] | I-49 | N-(2-((4-(3-(3- methoxyazetidin-1- yl)phenyl)thiazol- 2-yl)amino)- 2-oxoethyl)-1- (methylsulfonyl)- 1H-pyrrole-3- carboxamide | 651.4 |
| 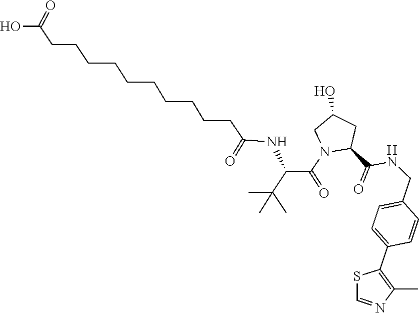 [SMILES: Cc1ncsc1-c1ccc(CNC(=O)[C@@H]2C[C@@H](O)CN2C(=O)[C@@H](NC(=O)CCCCCCCCCCC(=O)O)C(C)(C)C)cc1] | I-54 | 12-(((S)-1- ((2S,4R)-4-hydroxy- 2-((4-(4-methylthiazol-5- yl)benzyl)carbamoyl) pyrrolidin- 1-yl)-3,3-dimethyl- 1-oxobutan- 2-yl)amino)-12- oxododecanoic acid | 643.5 |

### Preparation of 7-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]heptanoic acid (I-50)

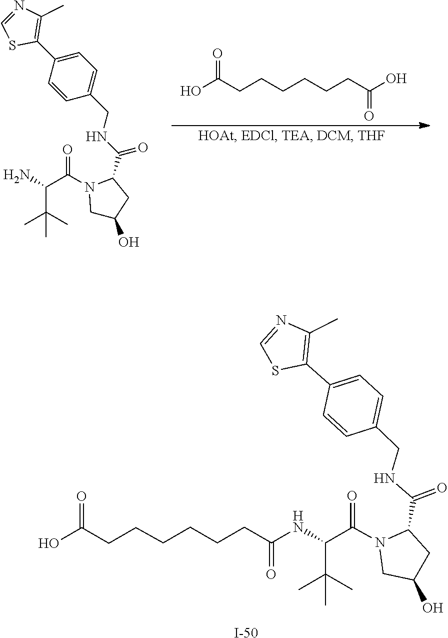

To a stirred solution of octanedioic acid (2.02 g, 11.6 mmol) in DCM (25.0 mL) and THF (25.0 mL) was added (2S,4R)-1-((S)-2-amino-3,3-dimethylbutanoyl)-4-hydroxy-N-(4-(4-methylthiazol-5-yl)benzyl)pyrrolidine-2-carboxamide (1.00 g, 2.32 mmol), TEA (823 mg, 8.13 mmol), HOAt (348 mg, 2.56 mmol), and EDCI (490 mg, 2.56 mmol) at 0° C. The resulting solution was stirred for 2 h at 0° C. The resulting mixture was concentrated under reduced pressure, and the residue purified by reverse phase flash chromatography to afford I-50 (900 mg, 66.0%) as a white solid. LCMS (ESI) m/z: [M+H]⁺=587.

### Preparation of 4-[[2-(2,6-Dioxopiperidin-3-yl)-1,3-dioxo-2,3-dihydro-1H-isoindol-4-yl]oxy]butanoic acid (I-29)

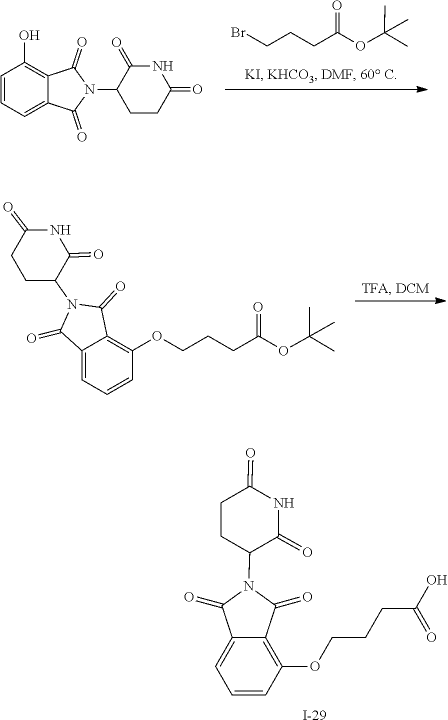

### Step 1: Preparation of tert-butyl 4-[[2-(2,6-dioxopiperidin-3-yl)-1,3-dioxoisoindol-4-yl]oxy] butanoate

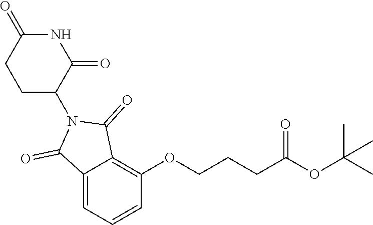 [SMILES: CC(C)(C)OC(=O)CCCOc1cccc2c1C(=O)N(C1CCC(=O)NC1=O)C2=O]

To a solution of 2-(2,6-dioxopiperidin-3-yl)-4-hydroxyisoindole-1,3-dione (2.00 g, 7.29 mmol) and tert-butyl 4-bromobutanoate (1.95 g, 8.752 mmol) in DMF (10.0 mL) was added KI (0.12 g, 0.729 mmol) and KHCO₃ (1.10 g, 10.9 mmol). The resulting solution was stirred at 60° C. for 5 h. The mixture was diluted with EtOAc and washed three times with water. The organic layer was dried over anhydrous sodium sulfate, filtered and concentrated to give a crude product. The crude product was purified by reverse phase C18 flash chromatography (Water:ACN) to give tert-butyl 4-[[2-(2,6-dioxopiperidin-3-yl)-1,3-dioxoisoindol-4-yl]oxy] butanoate (1.5 g, 49.4%) as an off-white solid. LCMS (ESI) m/z [M+H]⁺=417.

### Step 2: Preparation of 4-[[2-(2,6-dioxopiperidin-3-yl)-1,3-dioxo-2,3-dihydro-1H-isoindol-4-yl]oxy]butanoic acid (I-29)

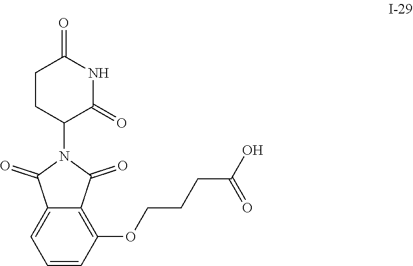 [SMILES: O=C(O)CCCOc1cccc2c1C(=O)N(C1CCC(=O)NC1=O)C2=O]

To a stirred solution of tert-butyl 4-[[2-(2,6-dioxopiperidin-3-yl)-1,3-dioxoisoindol-4-yl]oxy]butanoate (450 mg, 1.08 mmol) in DCM (5 mL) was added TFA (1 mL). The resulting solution was stirred for 2 h at 25° C. The resulting mixture was concentrated. This provided 1-29 (360 mg, 92.5%) as a white solid. ¹H NMR (400 MHz, Methanol-d₄) δ 7.79 (t, J=8.4, 7.4 Hz, 1H), 7.47 (d, J=7.8 Hz, 2H), 5.12 (dd, J=12.6, 5.5 Hz, 1H), 4.30 (t, J=6.2 Hz, 2H), 2.95-2.66 (m, 3H), 2.60 (t, J=7.3 Hz, 2H), 2.25-2.18 (m, 3H). LCMS (ESI) m/z: [M+H]⁺=361.10.

The following intermediates in Table 5 were prepared in a similar manner as described in the preparation of intermediate I-29 from the appropriate alkyl bromide.

TABLE 5

| | | | LCMS |
|---|---|---|---|
| | Intermediate | | (ESI)m/z: |
| Structure | No. | Name | [M + H]⁺ |
| 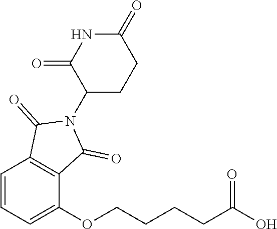 [SMILES: O=C(O)CCCCOc1cccc2c1C(=O)N(C1CCC(=O)NC1=O)C2=O] | I-30 | 5-((2-(2,6-dioxopiperidin-3-yl)- 1,3-dioxoisoindolin-4- yl)oxy)pentanoic acid | 375.1 |

### Preparation of 9-[[2-(2,6-dioxopiperidin-3-yl)-1,3-dioxoisoindol-5-yl]amino]nonanoic acid (I-3)

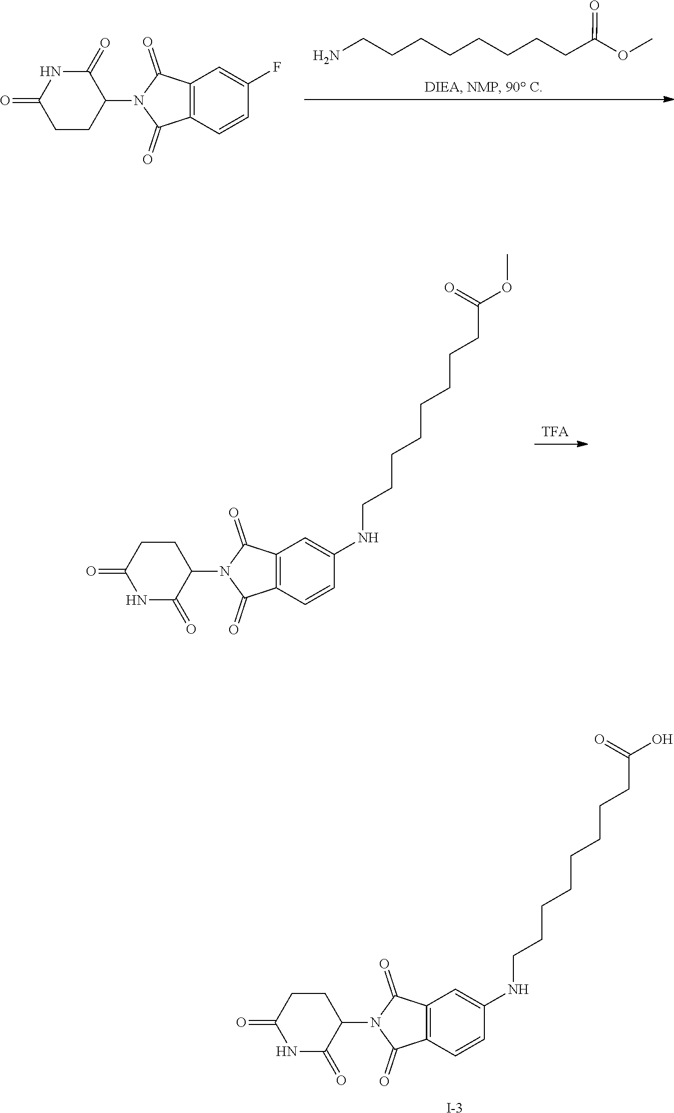

### Step 1: Preparation of methyl 9-[[2-(2,6-dioxopiperidin-3-yl)-1,3-dioxoisoindol-5-yl]amino]nonanoate

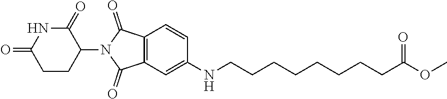 [SMILES: COC(=O)CCCCCCCCNc1ccc2c(c1)C(=O)N(C1CCC(=O)NC1=O)C2=O]

To a solution of 2-(2,6-dioxopiperidin-3-yl)-5-fluoroisoindole-1,3-dione (1.00 g, 3.62 mmol) and methyl 9-aminononanoate (814 mg, 4.34 mmol) in NMP (10.0 mL) was added DIEA (2.34 g, 18.1 mmol). The reaction mixture was heated to 90° C. under N₂ and for 5 h. The resulting mixture was diluted with water and was extracted three times with EtOAc. The combined organic layers were dried over anhydrous Na₂SO₄. After filtration, the filtrate was concentrated under reduced pressure. The residue was purified by silica gel column chromatography (PE/EtOAc) to afford methyl 9-[[2-(2,6-dioxopiperidin-3-yl)-1,3-dioxoisoindol-5-yl]amino]nonanoate (297 mg, 18.5%) as a yellow green solid; LCMS (ESI) m/z [M+H]⁺=444.

### Step 2: Preparation of 9-[[2-(2,6-dioxopiperidin-3-yl)-1,3-dioxoisoindol-5-yl]amino]nonanoic acid (I-3)

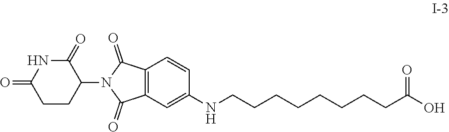 [SMILES: O=C(O)CCCCCCCCNc1ccc2c(c1)C(=O)N(C1CCC(=O)NC1=O)C2=O]

To a stirred solution of methyl 9-[[2-(2,6-dioxopiperidin-3-yl)-1,3-dioxoisoindol-5-yl]amino]nonanoate (330 mg, 0.744 mmol) in DCM (10 mL) was added TFA (10 mL) dropwise at room temperature. After stirring for 2 h, the resulting mixture was concentrated under reduced pressure and the residue was purified by flash chromatography to afford I-3 (290 mg, 90.8%) as a yellow solid. ¹H NMR (400 MHz, Methanol-d₄) δ 7.58 (d, 1H), 6.99 (d, 1H), 6.85 (dd, 1H), 5.06 (dd, 1H), 3.22 (t, 2H), 2.88-2.71 (m, 2H), 2.30 (t, 2H), 1.69-1.59 (m, 4H), 1.38 (s, 8H); LCMS (ESI) m/z: [M+H]⁺=430.19.

### Preparation (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (I-4)

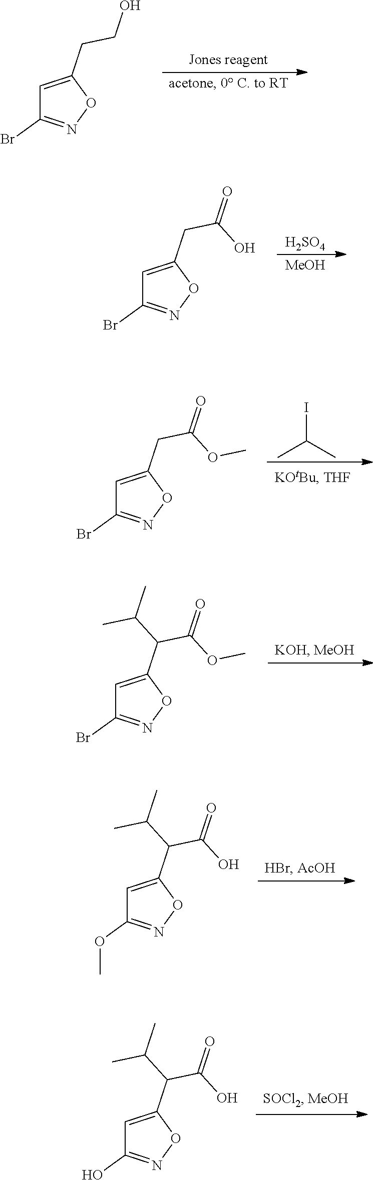

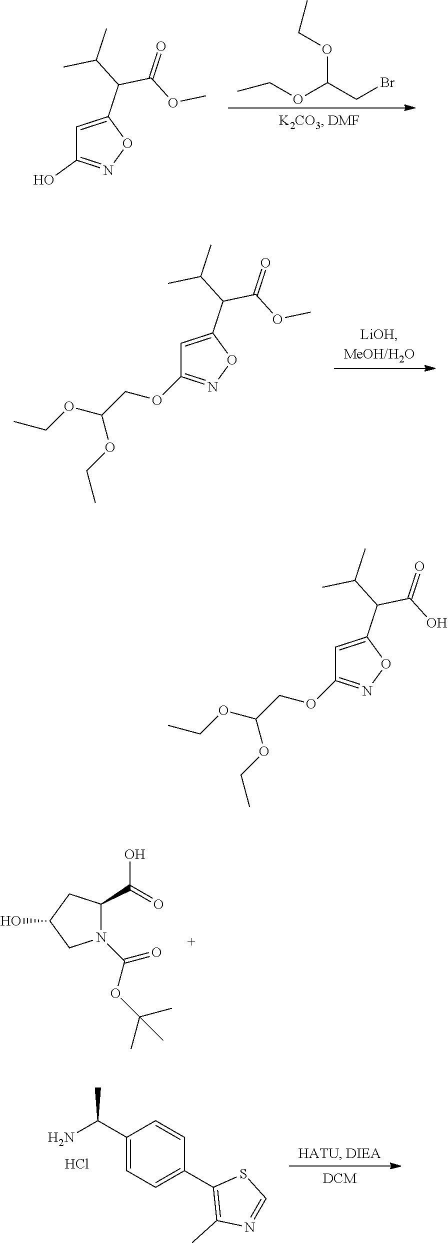

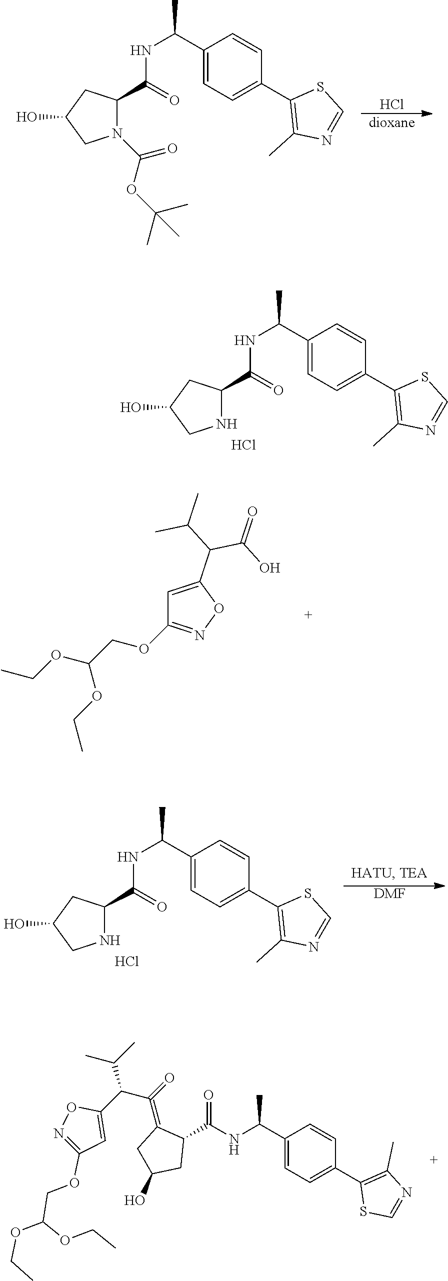

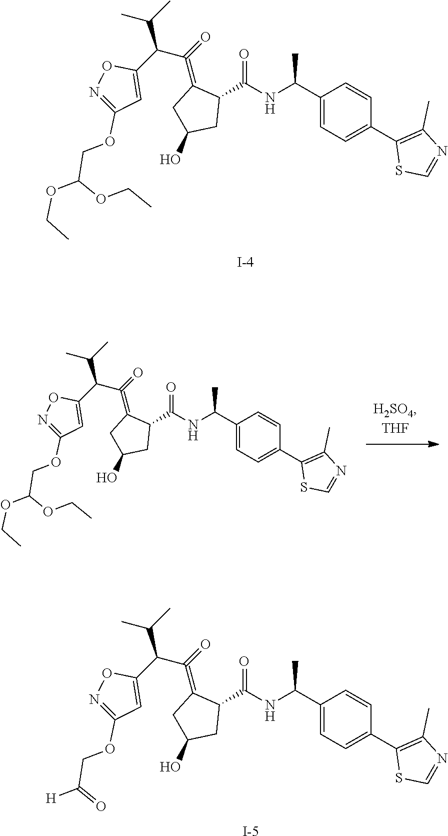

### Step 1: Preparation of 2-(3-bromoisoxazol-5-yl)acetic acid

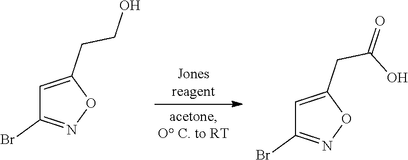 [SMILES: OCCc1cc(Br)no1 ; O=C(O)Cc1cc(Br)no1]

To a stirring solution of 2-(3-bromo-1,2-oxazol-5-yl)ethan-1-ol (30 g, 156 mmol) in acetone (389 mL) was added Jones reagent (2 M in acetone, 156 mL, 312 mmol) dropwise at 0° C. The resulting solution was stirred at 25° C. overnight. The mixture was diluted with water and extracted with EtOAc. The organic layer was washed with brine, and dried over anhydrous Na₂SO₄ and concentrated under reduced pressure to afford 2-(3-bromoisoxazol-5-yl)acetic acid as a brown solid (28 g, 86.5%). LCMS (ESI) m/z: [M+H]⁺=206.08 and 208.08.

### Step 2: Preparation of methyl 2-(3-bromoisoxazol-5-yl)acetate

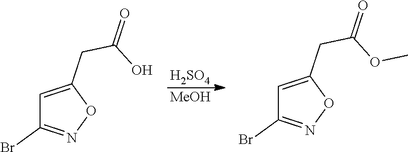

A solution of 2-(3-bromoisoxazol-5-yl)acetic acid (28 g, 135 mmol) and concentrated H₂SO₄ (3 mL, 72 mmol) in methanol (250 mL) was stirred at 70° C. for 2 h. The resulting solution was concentrated under reduced pressure. The residue was diluted with water and extracted with EtOAc. The organic layer was washed with brine, and dried over anhydrous MgSO₄ and concentrated under reduced pressure. The residue was purified by silica gel flash chromatography (EtOAc/petroleum ether) to afford methyl 2-(3-bromoisoxazol-5-yl)acetate as a white solid (23.4 g, 79%). LCMS (ESI) m/z: [M+H]⁺=219.90 and 221.86.

### Step 3: Preparation of methyl 2-(3-bromoisoxazol-5-yl)-3-methylbutanoate

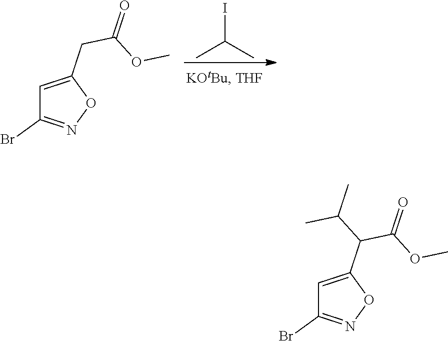

To a stirring solution of methyl 2-(3-bromoisoxazol-5-yl)acetate (23.4 g, 106 mmol) and KO^{t}Bu (17.8 g, 159 mmol) in THF (210 mL) was added 2-iodopropane (13.8 mL, 137 mmol) dropwise at 0° C. The reaction mixture was stirred at room temperature for 16 h and then quenched with water/ice. The resulting solution was extracted several times with EtOAc. The combined organic layers were washed with brine, and dried over anhydrous Na₂SO₄ and concentrated under reduced pressure. The residue was purified by silica gel flash chromatography (EtOAc/petroleum ether) to afford methyl 2-(3-bromoisoxazol-5-yl)-3-methylbutanoate as clear oil (16.7 g, 60%).

### Step 4: Preparation of 2-(3-methoxyisoxazol-5-yl)-3-methylbutanoic acid

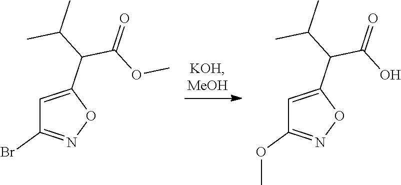

To a solution of methyl 2-(3-bromo-1,2-oxazol-5-yl)-3-methylbutanoate (16.7 g, 63.7 mmol) in methanol (130 mL) was added potassium hydroxide (35.7 g, 637 mmol). The mixture was stirred for 4 h at 100° C. The mixture was concentrated under vacuum and then diluted with water. The resulting solution was washed with EtOAc and pH of the aqueous layer was adjusted to pH 5 with 1 N HCl. This mixture was extracted several times with EtOAc. The combined organic layers were washed with brine and dried over anhydrous MgSO₄. The residue was purified by silica gel flash chromatography (EtOAc/petroleum ether) to afford 2-(3-methoxyisoxazol-5-yl)-3-methylbutanoic acid as yellow oil (8.8 g, 70%). LCMS (ESI) m/z: [M+H]⁺=200.15.

### Step 5: Preparation of 2-(3-hydroxyisoxazol-5-yl)-3-methylbutanoic acid

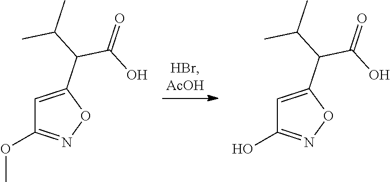

A solution of 2-(3-methoxyisoxazol-5-yl)-3-methylbutanoic acid (8.8 g, 44.1 mmol) in HOAc (80 mL) and HBr (80 mL) was stirred at 60° C. for 16 h. The resulting mixture was concentrated under reduced pressure to afford crude 2-(3-hydroxyisoxazol-5-yl)-3-methylbutanoic acid (8.16 g, quant.)

### Step 6: Preparation of methyl 2-(3-hydroxyisoxazol-5-yl)-3-methylbutanoate

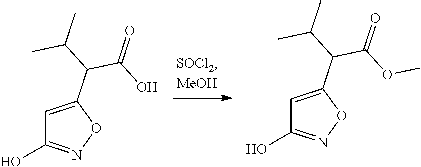

A solution of 2-(3-hydroxy-1,2-oxazol-5-yl)-3-methylbutanoic acid (8.16 g, 44.0 mmol) in methanol (30 mL) was slowly added SOCl₂ (14.2 mL, 197 mmol). The mixture was stirred at room temperature for 3 h. The solvent was removed under reduced pressure. The residue was dilute with water and extracted with EtOAc. The organic layer was washed with brine, and dried over anhydrous Na₂SO₄ and concentrated under reduced pressure. The residue was purified by silica gel flash chromatography (MeOH/DCM) to afford methyl 2-(3-hydroxyisoxazol-5-yl)-3-methylbutanoate as a clear oil (7.79 g, 89%). LCMS (ESI) m/z: [M+H]⁺=200.15.

### Step 7: Preparation of methyl 2-(3-(2,2-diethoxyethoxy)isoxazol-5-yl)-3-methylbutanoate

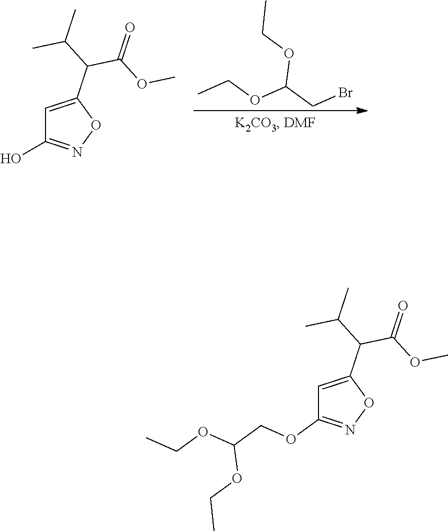

To a solution of methyl 2-(3-hydroxy-1,2-oxazol-5-yl)-3-methylbutanoate (7.79 g, 39.1 mmol) in DMF (90 mL) was added 2-bromo-1,1-diethoxyethane (8.77 mL, 58.6 mmol) and potassium carbonate (10.8 g, 78.2 mmol). The reaction was stirred at 70° C. overnight. The reaction mixture was cooled and then water was added to the mixture. The resulting mixture was extracted with EtOAc several times. The combined organic layers were wash with brine and dried over anhydrous MgSO₄. Solvent was removed under reduced pressure and the resulting residue was purified by silica gel flash chromatography (EtOAc:heptane) to afford methyl 2-(3-(2,2-diethoxyethoxy)isoxazol-5-yl)-3-methylbutanoate as a colorless oil (7.8 g, 63%). LCMS (ESI) m/z: [M-C₂H₅O]⁺=270.30.

### Step 8: Preparation of 2-(3-(2,2-diethoxyethoxy)isoxazol-5-yl)-3-methylbutanoic acid

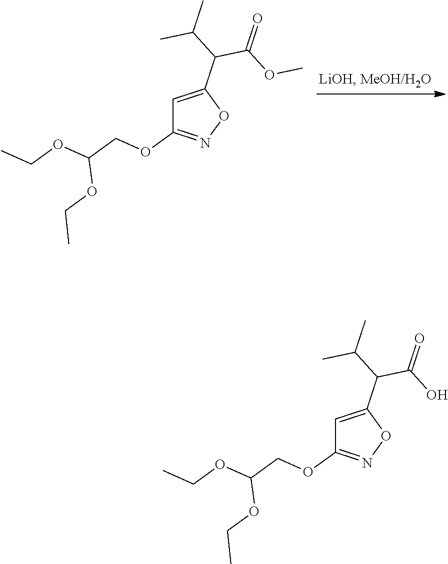

To a solution of methyl 2-[3-(2,2-diethoxyethoxy)-1,2-oxazol-5-yl]-3-methylbutanoate (7.8 g, 24.7 mmol) in methanol (50 mL) and water (25 mL) was added lithium hydroxide mono hydrate (4.14 g, 98.8 mmol). The reaction was stirred at 40° C. for 2 h. The pH was adjusted to 4-5 with 1 N HCl. The mixture was extract with ethyl acetate several times and combined organic layers were dried organic over MgSO₄. Solvent were removed under reduced pressure and the residue was purified by silica gel flash chromatography (DCM:MeOH) to afford 2-(3-(2,2-diethoxyethoxy)isoxazol-5-yl)-3-methylbutanoic acid as a colorless oil (6.1 g, 89%). LCMS (ESI) m/z: [M−H]⁻=300.21.

### Step 9: Preparation of tert-butyl (2S,4R)-4-hydroxy-2-(((S)-1-(4-(4-methylthiazol-5-yl)phenyl)ethyl)carbamoyl) pyrrolidine-1-carboxylate

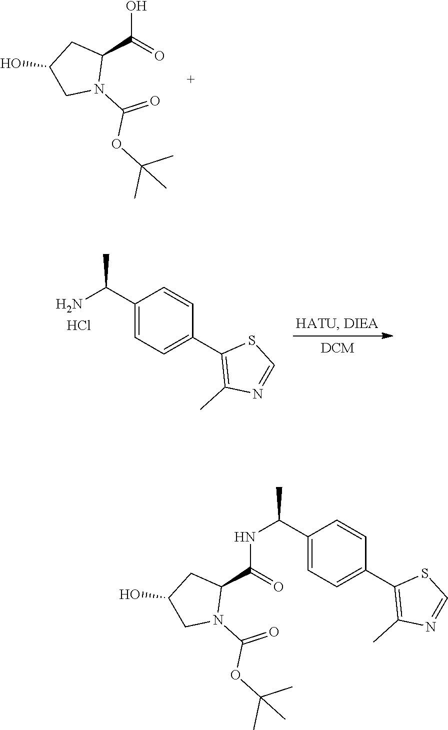 [SMILES: CC(C)(C)OC(=O)N1C[C@H](O)C[C@H]1C(=O)O ; Cl ; Cc1ncsc1-c1ccc([C@H](C)N)cc1 ; Cc1ncsc1-c1ccc([C@H](C)NC(=O)[C@@H]2C[C@@H](O)CN2C(=O)OC(C)(C)C)cc1]

To a solution of (S)—I-(4-(4-methylthiazol-5-yl)phenyl)ethan-1-amine hydrochloride (5.0 g, 19.6 mmol) and (2S,4R)-1-[(tert-butoxy)carbonyl]-4-hydroxy pyrrolidine-2-carboxylic acid (4.47 g, 20.5 mmol) in DCM (70 mL) at 0° C. was added HATU (8.98 g, 23.5 mmol) followed by dropwise addition of DIEA (16.4 mL, 98.0 mmol). After stirring for 16 h at room temperature, the reaction mixture was poured into ice water. The resulting mixture was extracted several times with DCM. The combined organic layers were washed with water, brine, and dried over anhydrous Na₂SO₄ and concentrated under vacuum. The resulting residue was purified by silica gel flash chromatography (MeOH:DCM) to afford tert-butyl (2S,4R)-4-hydroxy-2-(((S)-1-(4-(4-methylthiazol-5-yl)phenyl)ethyl)carbamoyl) pyrrolidine-1-carboxylate (8.33 g, 98%). LCMS (ESI) m/z: [M+H]⁺=432.38.

### Step 10: Preparation of (2S,4R)-4-hydroxy-N—((S)-1-(4-(4-methylthiazol-5-yl)phenyl)ethyl)pyrrolidine-2-carboxamide hydrochloride

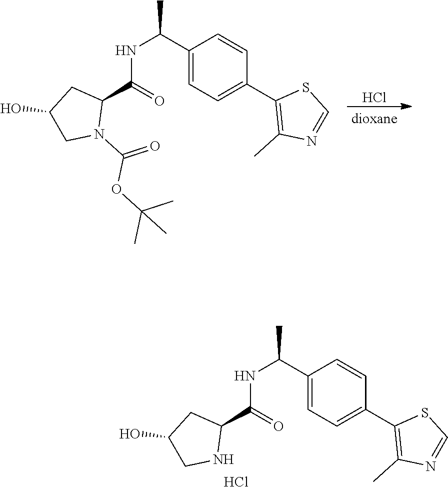 [SMILES: Cc1ncsc1-c1ccc([C@H](C)NC(=O)[C@@H]2C[C@@H](O)CN2C(=O)OC(C)(C)C)cc1 ; Cl ; Cc1ncsc1-c1ccc([C@H](C)NC(=O)[C@@H]2C[C@@H](O)CN2)cc1 ; Cl]

To a stirred solution of tert-butyl (2S,4R)-4-hydroxy-2-(((S)—I-(4-(4-methylthiazol-5-yl)phenyl)ethyl)carbamoyl)pyrrolidine-1-carboxylate (8.33 g, 19.3 mmol) at 0° C. was added a solution of HCl in 1,4-dioxane (4 N, 50 mL, 200 mmol) resulting in a sticky yellow gum. 15 mL of MeOH was added to the mixture and the mixture was stirred at room temperature for 2 h. The solvents were removed under reduced pressure and the residue was washed with diethyl ether to afford (2S,4R)-4-hydroxy-N—((S)-1-(4-(4-methylthiazol-5-yl)phenyl)ethyl)pyrrolidine-2-carboxamide hydrochloride which was used in the next step without further purification.

### Step 11: Preparation of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (I-4)

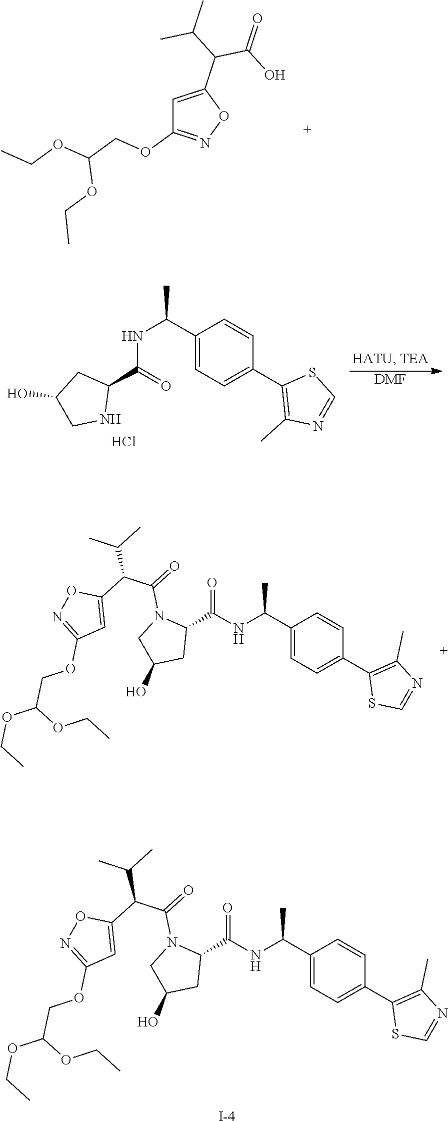

To a solution of 2-[3-(2,2-diethoxyethoxy)isoxazol-5-yl]-3-methyl-butanoic acid (5.75 g, 19.0 mmol) in DMF (30 mL) was added HATU (8.6 g, 22.7 mmol). After stirring at 20° C. for 0.5 h, a solution of (2S,4R)-4-hydroxy-N-[(1S)—I-[4-(4-methylthiazol-5-yl)phenyl]ethyl]pyrrolidine2-carboxamide hydrochloride (6.97 g, 19.0 mmol) and triethylamine (7.92 mL, 56.9 mmol) in DMF (20 mL) was added to the mixture and the resulting mixture was stirred at 20° C. The reaction mixture was quenched by addition water and extracted several times with EtOAc. The combined organic layers were washed with brine, and dried over anhydrous MgSO₄, filtered and concentrated under reduced pressure. The residue was purified by silica gel flash chromatography (DCM:MeOH) to afford (2S,4R)—I-[2-[3-(2,2-diethoxyethoxy)isoxazol-5-yl]-3-methyl-butanoyl]-4-hydroxy-N—[(I S)-1-[4-(4-methylthiazol-5-yl)phenyl]ethyl]pyrrolidine-2-carboxamide (10 g, 16.2 mmol) as a white solid. The mixture of diastereomers were separated by chiral SFC chromatography to afford (2S,4R)-1-((S)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide and (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide.

(2S,4R)-1-((S)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide peak 1: (2.2 g, 19%). LCMS (ESI) m/z [M+H]⁺=615.4.

(2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (I-4) peak 2: (2.5 g, 21%). LCMS (ESI) m/z [M+H]⁺=615.4.

### Step 12: Preparation of (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (I-5)

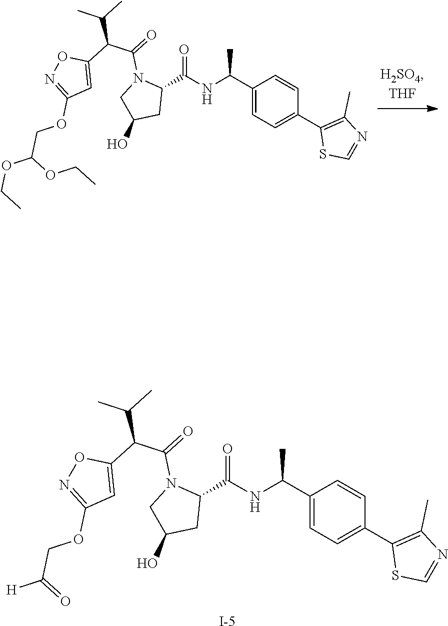

To a stirred solution of H₂SO₄ (1 N, 6.00 mL) and THF (6.00 mL) was added (2S,4R)-1-[(2R)-2-[3-(2-ethoxy-2-methoxyethoxy)-1,2-oxazol-5-yl]-3-methylbutanoyl]-4-hydroxy-N-[(1S)-1-[4-(4-methyl-1,3-thiazol-5-yl)phenyl]ethyl]pyrrolidine-2-carboxamide (300 mg, 0.499 mmol) dropwise portions at room temperature. The resulting mixture was stirred for 8 h at 50° C. The resulting mixture was diluted with water, then neutralized to pH ˜7 with saturated aqueous NaHCO₃. The resulting mixture was extracted three times with EtOAc. The combined organic layers were washed twice with brine and dried over anhydrous Na₂SO₄. After filtration, the filtrate was concentrated under reduced pressure to afford (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (I-5, 256 mg, 97.3%) as a white solid. LCMS (ESI) m/z: [M+H]⁺=541.

### Preparation of methyl 2-[4-chloro-3-(2,2-diethoxyethoxy)-1,2-oxazol-5-yl]-3-methylbutanoate (I-59)

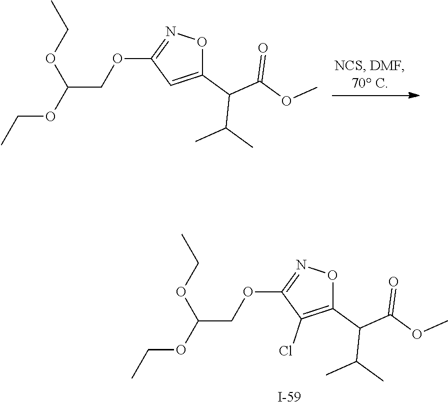

A solution of methyl 2-(3-(2,2-diethoxyethoxy)isoxazol-5-yl)-3-methylbutanoate (300 mg, 0.951 mmol, 1.00 equiv) and NCS (152.43 mg, 1.141 mmol, 1.2 equiv) in DMF (3.00 mL) was stirred for 12 h at 70 degrees C. The residue was purified by reverse phase flash chromatography with the following conditions: column, C18 silica gel; mobile phase, CH₃CN in water (0.05% FA), 0% to 100% gradient in 25 min; detector, UV 254 nm. This resulted in I-59 (180 mg, 54.09%) as a white solid. LCMS (ESI) m/z: [M+H]⁺=350.

The following intermediates in Table 6 were prepared in a similar manner as described in the preparation of intermediate I-5 starting with methyl 2-(3-hydroxy-1,2-oxazol-5-yl)-3-methylbutanoate and the appropriate alkyl bromides.

TABLE 6

| | Intermediate | | LCMS |
|---|---|---|---|
| Structure | No. | Name | (ESI) m/z |
| 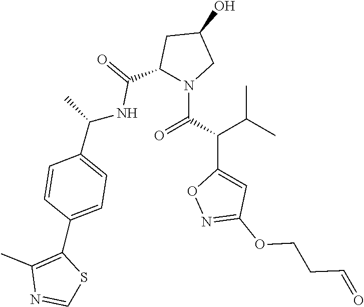 [SMILES: Cc1ncsc1-c1ccc([C@H](C)NC(=O)[C@@H]2C[C@@H](O)CN2C(=O)[C@@H](c2cc(OCCC=O)no2)C(C)C)cc1] | I-60 | (2S,4R)-4-hydroxy-1-((R)-3- methyl-2-(3-(3- oxopropoxy)isoxazol-5- yl)butanoyl)-N-((S)-1-(4-(4- methylthiazol-5- yl)phenyl)ethyl)pyrrolidine-2- carboxamide | 555 |
| 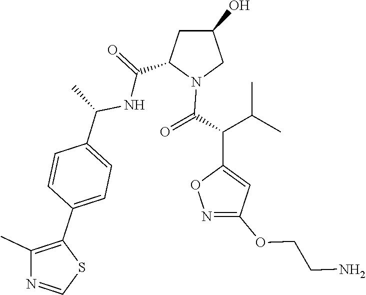 [SMILES: Cc1ncsc1-c1ccc([C@H](C)NC(=O)[C@@H]2C[C@@H](O)CN2C(=O)[C@@H](c2cc(OCCN)no2)C(C)C)cc1] | I-61 | (2S,4R)-1-((R)-2-(3-(2- aminoethoxy)isoxazol-5-yl)-3- methylbutanoyl)-4-hydroxy-N- ((S)-1-(4-(4-methylthiazol-5- yl)phenyl)ethyl)pyrrolidine-2- carboxamide | 542 |
| 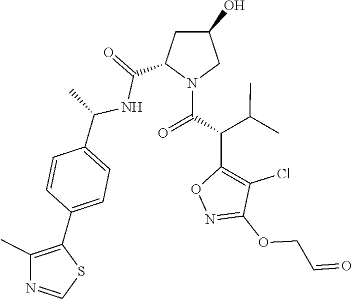 [SMILES: Cc1ncsc1-c1ccc([C@H](C)NC(=O)[C@@H]2C[C@@H](O)CN2C(=O)[C@@H](c2onc(OCC=O)c2Cl)C(C)C)cc1] | I-62 | (2S,4R)-1-((R)-2-(4-chloro-3- (2-oxoethoxy)isoxazol-5-yl)-3- methylbutanoyl)-4-hydroxy-N- ((S)-1-(4-(4-methylthiazol-5- yl) phenyl)ethyl)pyrrolidine-2- carboxamide | 575 |
| 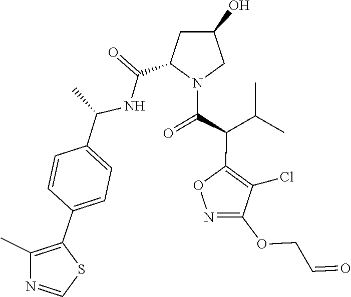 [SMILES: Cc1ncsc1-c1ccc([C@H](C)NC(=O)[C@@H]2C[C@@H](O)CN2C(=O)[C@H](c2onc(OCC=O)c2Cl)C(C)C)cc1] | I-63 | (2S,4R)-1-((S)-2-(4-chloro-3- (2-oxoethoxy)isoxazol-5-yl)-3- methylbutanoyl)-4-hydroxy-N- ((S)-1-(4-(4-methylthiazol-5- yl)phenyl)ethyl)pyrrolidine-2- carboxamide | 575 |
| 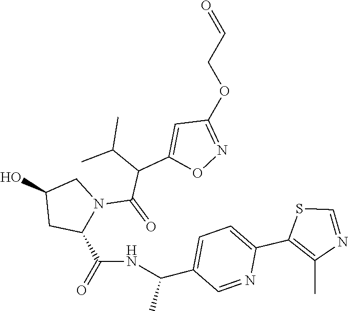 [SMILES: Cc1ncsc1-c1ccc([C@H](C)NC(=O)[C@@H]2C[C@@H](O)CN2C(=O)C(c2cc(OCC=O)no2)C(C)C)cn1] | I-64 | (2S,4R)-4-hydroxy-1-(3- methyl-2-(3-(2- oxoethoxy)isoxazol-5-yl) butanoyl)-N-((S)-1-(6-(4- methylthiazol-5-yl) pyridin-3- yl)ethyl) pyrrolidine-2- carboxamide | 542 |
| 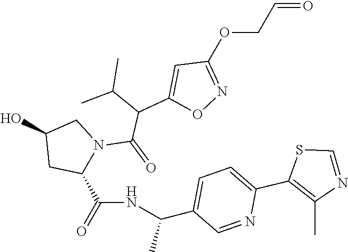 [SMILES: Cc1ncsc1-c1ccc([C@H](C)NC(=O)[C@@H]2C[C@@H](O)CN2C(=O)C(c2cc(OCC=O)no2)C(C)C)cn1] | I-65 | (2S,4R)-4-hydroxy-N-{[4-(4- methyl-1,3-thiazol-5- yl)phenyl]methyl}-1-[(2R)-3- methyl-2-[3-(2-oxoethoxy)-1,2- oxazol-5- yl]butanoyl]pyrrolidine-2- carboxamide | 527 |
| 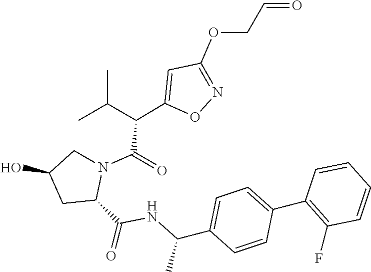 [SMILES: CC(C)[C@@H](C(=O)N1C[C@H](O)C[C@H]1C(=O)N[C@@H](C)c1ccc(-c2ccccc2F)cc1)c1cc(OCC=O)no1] | I-66 | (2S,4R)-N-((S)-1-(2′-fluoro-[1,1′- biphenyl]-4-yl)ethyl)-4-hydroxy- 1-((S)-3-methyl-2-(3-(2- oxoethoxy)isoxazol-5- yl)butanoyl)pyrrolidine-2- carboxamide | 538 |

### Preparation of 2-((5-((R)-1-((2S,4R)-4-hydroxy-2-(((S)-1-(4-(2-methylthiazol-5-yl)phenyl)ethyl)carbamoyl)pyrrolidin-1-yl)-3-methyl-1-oxobutan-2-yl)isoxazol-3-yl)oxy)acetic acid (I-67)

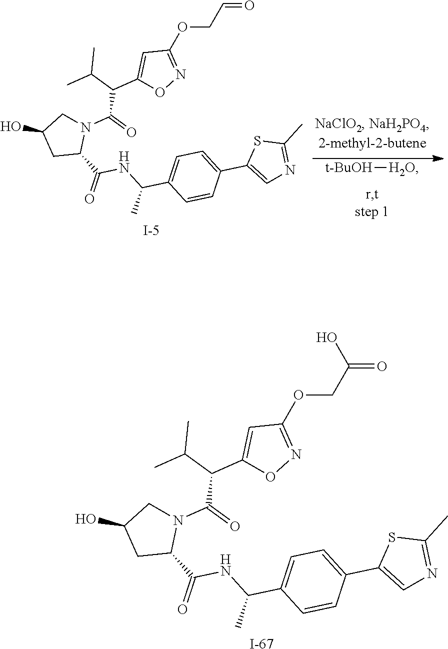

To a stirred solution of (2S,4R)-4-hydroxy-N-[(1S)-1-[4-(2-methyl-1,3-thiazol-5-yl)phenyl] ethyl]-1-[(2S)-3-methyl-2-[3-(2-oxoethoxy)-1,2-oxazol-5-yl]butanoyl]pyrrolidine-2-carboxamide (30.00 mg, 0.055 mmol, 1.00 equiv) and 2-methyl-2-butene (0.78 mg, 0.011 mmol, 0.20 equiv) in ter-butanol (2 mL) was added dropwise a solution of NaClO₂ (50.19 mg, 0.550 mmol, 10.00 equiv) and Na₂HPO₄ (78.77 mg, 0.550 mmol, 10.00 equiv) in water (2.00 mL) at 0° C. The mixture was stirred at 0° C. for 0.5 h, then warmed up to room temperature. and stirred for an additional 1.5 h. The reaction was quenched by addition of a mixture of saturated Na₂S₂O₃ solution and brine, extracted with CHCl₃ (20 mL×3). The combined organic extracts were dried over Na₂SO₄, concentrated in vacuo and purified by silica gel chromatography (PE/EtOAc=1/3 to 1/1). This provided intermediate I-67 (15.80 mg, 49.93%) as a colorless oil. LCMS (ESI) m/z: [M+H]⁺=557.

### Preparation of (2S,4R)-4-hydroxy-1-((R)-3-methyl-2-(3-(2-oxoethoxy)isoxazol-5-yl)butanoyl)-N-((3-(4-methylthiazol-5-yl)bicyclo[1.1.1]pentan-1-yl)methyl)pyrrolidine-2-carboxamide (I-68)

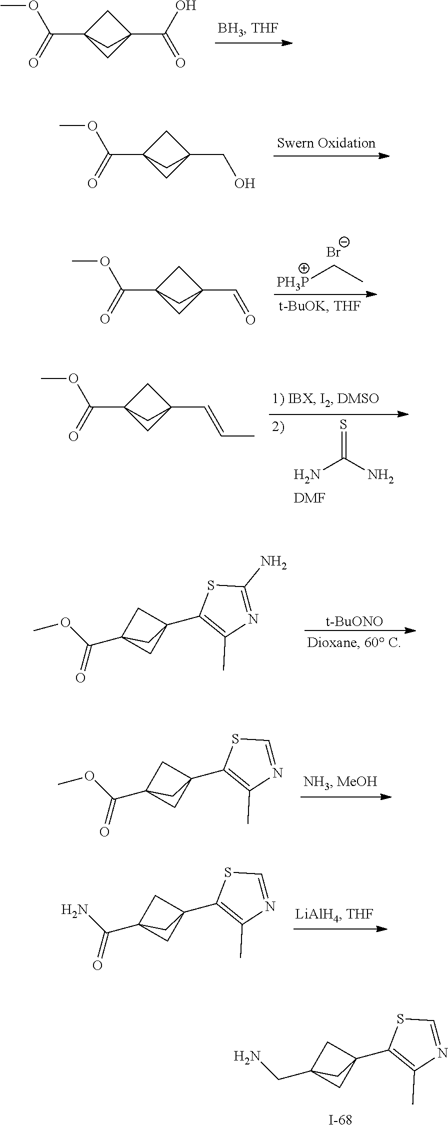

### Step 1: Preparation of methyl 3-(hydroxymethyl)bicyclo[1.1.1]pentane-1-carboxylate

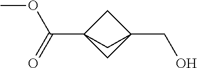

A solution of 3-(methoxycarbonyl)bicyclo[1.1.1]pentane-1-carboxylic acid (5.00 g, 29.383 mmol, 1.00 equiv) in THF (50.00 mL) was treated with borane (0.61 g, 0.044 mmol, 1.50 equiv). The resulting mixture was stirred overnight at room temperature. The reaction was quenched with water at 0 degrees C. and was extracted with EtOAc (3×500 mL). The combined organic layers were washed with brine (2×100 mL), and dried over anhydrous Na₂SO₄. After filtration, the filtrate was concentrated under reduced pressure. The crude product was used in the next step directly without further purification. LCMS (ESI) m/z: [M+H]⁺=157.

### Step 2: Preparation of methyl 3-formylbicyclo[1.1.1]pentane-1-carboxylate

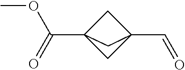

To a stirred mixture of oxalyl chloride (1.22 g, 9.60 mmol, 1.50 equiv) in DCM (20.00 mL) was added DMSO (1.5 g, 19.21 mmol, 3.00 equiv) dropwise at −78 degrees C. under an atmosphere of dry nitrogen. The resulting mixture was stirred for 15 min at −78 degrees C. under an atmosphere of dry nitrogen. To the above mixture was added methyl 3-(hydroxymethyl)bicyclo[1.1.1]pentane-1-carboxylate (1.00 g, 6.40 mmol, 1.00 equiv) at −78 degrees C. The resulting mixture was stirred for additional 30 min at −78 degrees C. To the above mixture was added Et₃N (3.89 g, 38.42 mmol, 6.00 equiv) at −78 degrees C. The resulting mixture was stirred for additional 30 min at −78 degrees C. The resulting mixture was extracted with EtOAc (3×200 mL). The combined organic layers were washed with brine (100 mL), and dried over anhydrous Na₂SO₄. After filtration, the filtrate was concentrated under reduced pressure to afford methyl 3-formylbicyclo[1.1.1]pentane-1-carboxylate (400 mg, 32.42%) as yellow solid. LCMS (ESI) m/z: [M+H]⁺=155.

### Step 3: Preparation of methyl (E)-3-(prop-1-en-1-yl)bicyclo[1.1.1]pentane-1-carboxylate

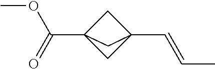

To a stirred solution of ethyltriphenylphosphanium bromide (13.00 g, 35.028 mmol, 3 equiv) in THE (150 mL) was added t-BuOK (3.28 g, 29.190 mmol, 2.5 equiv) at 0 degrees C. The resulting mixture was stirred for 1 h at 0 degrees C. To the above mixture was added methyl 3-formylbicyclo[1.1.1]pentane-1-carboxylate (1.8 g, 11.676 mmol, 1.00 equiv) in THF (10 mL) dropwise over 15 min at 0 degrees C. The resulting mixture was stirred for additional 3 h at room temperature under N₂ atmosphere. The mixture was acidified to pH 7 with saturated NH₄Cl (aq.). The resulting mixture was extracted with EtOAc (3×200 mL). The combined organic layers were washed with brine (100 mL), and dried over anhydrous Na₂SO₄. After filtration, the filtrate was concentrated under reduced pressure. The residue was purified by silica gel column chromatography, eluted with PE/EA (10:1) to afford methyl (E)-3-(prop-1-en-1-yl)bicyclo[1.1.1]pentane-1-carboxylate (1.16 g, 60%) as yellow oil. LCMS (ESI) m/z: [M+H]⁺=167.

### Step 4: Preparation of methyl 3-(2-amino-4-methylthiazol-5-yl)bicyclo[1.1.1]pentane-1-carboxylate

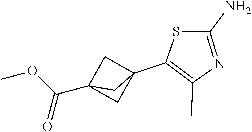 [SMILES: COC(=O)C12CC(c3sc(N)nc3C)(C1)C2]

To a stirred solution of IBX (3.033 g, 10.80 mmol, 2 equiv) and I₂ (1.59 g, 6.00 mmol, 1.1 equiv) in DMSO (100 mL) was added methyl (E)-3-(prop-1-en-1-yl)bicyclo[1.1.1]pentane-1-carboxylate (900 mg, 5.40 mmol, 1.00 equiv) in one charge at room temperature. The reaction mixture was stirred at room temperature until full consumption of the starting alkene (monitored by LCMS). Then it was diluted with DCM (100 mL) and washed with saturated aqueous NaHCO₃—Na₂S₂O₃. The aqueous layer was extracted with DCM (2×100 mL); the combined organic layers were dried over Na₂SO₄ and filtered. The thiourea (1.24 g, 16.20 mmol, 3 equiv) and dimethylformamide (100 mL) were added to the above mixture. The reaction mixture was stirred at room temperature for 12 h. The reaction mixture was washed with water (3×300 mL), and dried over anhydrous Na₂SO₄. After filtration, the filtrate was concentrated under reduced pressure. The residue was purified by silica gel column chromatography, eluted with PE/EA (2:1) to afford methyl 3-(2-amino-4-methylthiazol-5-yl)bicyclo[1.1.1]pentane-1-carboxylate (670.6 mg, 52.0%) as yellow oil. LCMS (ESI) m/z: [M+H]⁺=239.

### Step 5: Preparation of methyl 3-(4-methylthiazol-5-yl)bicyclo[1.1.1]pentane-1-carboxylate

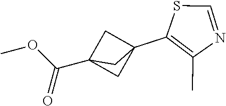

To a stirred solution of methyl 3-(2-amino-4-methylthiazol-5-yl)bicyclo[1.1.1]pentane-1-carboxylate (654 mg, 2.75 mmol, 1.00 equiv) in THF (50 mL) was added t-BuNO₂ (1.41 g, 13.74 mmol, 5 equiv) at room temperature. The resulting mixture was stirred for 1 h at 60 degrees C. The mixture was allowed to cool down to room temperature and concentrated under reduced pressure. The residue was purified by silica gel column chromatography, eluted with PE/EA (3:1) to afford methyl 3-(4-methylthiazol-5-yl)bicyclo[1.1.1]pentane-1-carboxylate (145.8 mg, 23.5%) as yellow oil. LCMS (ESI) m/z: [M+H]⁺=224.

### Step 6: Preparation of 3-(4-methylthiazol-5-yl)bicyclo[1.1.1]pentane-1-carboxamide

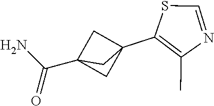

To a stirred solution of methyl 3-(4-methyl-1,3-thiazol-5-yl)bicyclo[1.1.1]pentane-1-carboxylate (144 mg, 0.645 mmol, 1.00 equiv) was added ammonia in methanol (15 mL) at room temperature. The resulting mixture was stirred for 16 h at 50 degrees C. The resulting mixture was concentrated under reduced pressure. This resulted in 3-(4-methylthiazol-5-yl)bicyclo[1.1.1]pentane-1-carboxamide (72 mg, 53.60%) as a yellow solid. LCMS (ESI) m/z: [M+H]⁺=209.

### Step 7: Preparation of (3-(4-methylthiazol-5-yl)bicyclo[1.1.1]pentan-1-yl)methanamine

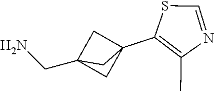

To a stirred solution of 3-(4-methylthiazol-5-yl)bicyclo[1.1.1]pentane-1-carboxamide (72 mg, 0.346 mmol, 1.00 equiv) in THF (2 mL) was added LiAlH₄ (0.3 mL, 2.5 M) at 0 degrees C. under an atmosphere of dry nitrogen. The resulting mixture was stirred for 16 h at room temperature under an atmosphere of dry nitrogen. The mixture was acidified to pH 7 with saturated NH₄Cl (aq.). The resulting mixture was extracted with EtOAc (3×20 mL). The combined organic layers were washed with brine (100 mL), and dried over anhydrous Na₂SO₄. After filtration, the filtrate was concentrated under reduced pressure. The residue was purified by silica gel column chromatography, eluted with PE/EA (1:1) to afford (3-(4-methylthiazol-5-yl)bicyclo[1.1.1]pentan-1-yl)methanamine (60.3 g, 90.00%) as yellow oil. LCMS (ESI) m/z: [M+H]⁺=195.

### Preparation of (2S,4R)-4-hydroxy-1-((R)-3-methyl-2-(3-(2-oxoethoxy)isoxazol-5-yl)butanoyl)-N-((3-(4-methylthiazol-5-yl)bicyclo[1.1.1]pentan-1-yl)methyl)pyrrolidine-2-carboxamide (I-69)

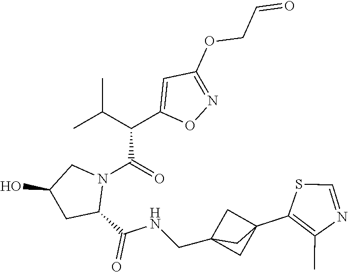 [SMILES: Cc1ncsc1C12CC(CNC(=O)[C@@H]3C[C@@H](O)CN3C(=O)[C@@H](c3cc(OCC=O)no3)C(C)C)(C1)C2]

(2S,4R)-4-hydroxy-1-((R)-3-methyl-2-(3-(2-oxoethoxy)isoxazol-5-yl)butanoyl)-N-((3-(4-methylthiazol-5-yl)bicyclo[1.1.1]pentan-1-yl)methyl)pyrrolidine-2-carboxamide was prepared in a similar manner as described in the preparation of intermediate I-5 starting with (3-(4-methylthiazol-5-yl)bicyclo[1.1.1]pentan-1-yl)methanamine. LCMS (ESI) m/z: [M+H]⁺=517.

### Preparation of (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (I-70) and (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-71)

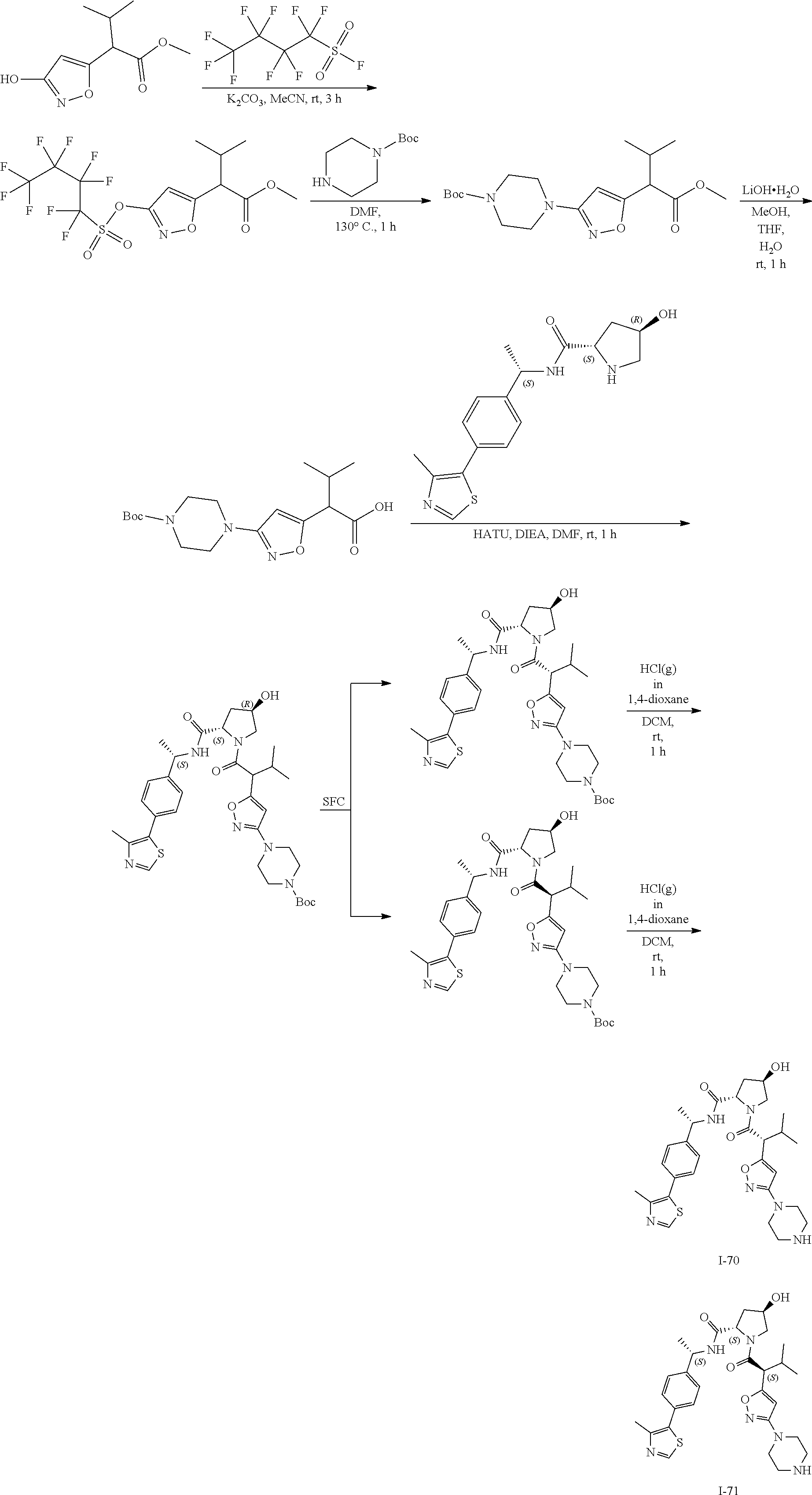

### Step 1: Preparation of methyl 3-methyl-2-[3-[(1,1,2,2,3,3,4,4,4-nonafluorobutanesulfonyl)oxy]-1,2-oxazol-5-yl]butanoate

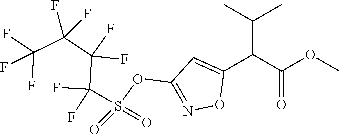 [SMILES: COC(=O)C(c1cc(OS(=O)(=O)C(F)(F)C(F)(F)C(F)(F)C(F)(F)F)no1)C(C)C]

To a stirred solution of methyl 2-(3-hydroxy-1,2-oxazol-5-yl)-3-methylbutanoate (100.00 mg, 0.502 mmol, 1.00 equiv) in MeCN (0.50 mL) was added perfluorobutanesulfonyl fluoride (303.29 mg, 1.004 mmol, 2.00 equiv) and K₂CO₃ (208.13 mg, 1.506 mmol, 3.00 equiv) at room temperature. The resulting mixture was stirred for 3 h, then carefully quenched with water at 0 degrees C. The resulting mixture was extracted with EA (2×50 mL). The combined organic layers were washed with brine (50 mL) and dried over anhydrous Na₂SO₄. After filtration, the filtrate was concentrated under reduced pressure. The residue was purified by silica gel column chromatography, eluting with PE/EA (2/1) to afford methyl 3-methyl-2-[3-[(1,1,2,2,3,3,4,4,4-nonafluorobutanesulfonyl)oxy]-1,2-oxazol-5-yl]butanoate (217 mg, crude) as a white solid. LCMS (ESI) m/z: [M+H]⁺=482.

### Step 2: Preparation of tert-butyl 4-[5-(1-methoxy-3-methyl-1-oxobutan-2-yl)-1,2-oxazol-3-yl]piperazine-1-carboxylate

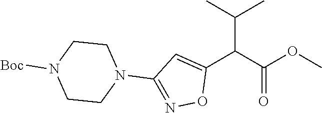 [SMILES: COC(=O)C(c1cc(N2CCN(C(=O)OC(C)(C)C)CC2)no1)C(C)C]

To a stirred solution of methyl 3-methyl-2-[3-[(1,1,2,2,3,3,4,4,4-nonafluorobutanesulfonyl)oxy]-1,2-oxazol-5-yl]butanoate (217.00 mg, 0.451 mmol, 1.00 equiv) in DMF (3.00 mL) was added tert-butyl piperazine-1-carboxylate (83.98 mg, 0.451 mmol, 1.00 equiv) at room temperature. The resulting mixture was stirred for 1 h at 130° C. The mixture was allowed to cool down to room temperature. The residue was purified by reverse phase flash chromatography with the following conditions: column, C18 silica gel; mobile phase, MeCN in water (0.1% FA), 0 to 100% gradient in 30 min. This provided tert-butyl 4-[5-(1-methoxy-3-methyl-1-oxobutan-2-yl)-1,2-oxazol-3-yl]piperazine-1-carboxylate (54 mg, 32.59%) as a yellow oil. LCMS (ESI) m/z: [M+H]⁺=368.

### Step 3: Preparation of 2-[3-[4-(tert-butoxycarbonyl)piperazin-1-yl]-1,2-oxazol-5-yl]-3-methylbutanoic acid

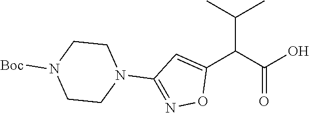 [SMILES: CC(C)C(C(=O)O)c1cc(N2CCN(C(=O)OC(C)(C)C)CC2)no1]

To a stirred solution of tert-butyl 4-[5-(1-methoxy-3-methyl-1-oxobutan-2-yl)-1,2-oxazol-3-yl]piperazine-1-carboxylate (54.00 mg, 0.147 mmol, 1.00 equiv) in MeOH (0.80 mL) was added THF (0.80 mL) and H₂O (0.80 mL) at room temperature, followed by addition of LiOH·H₂O (18.50 mg, 0.441 mmol, 3.00 equiv). The resulting mixture was stirred for an additional 1 h at room temperature. The mixture was acidified to pH 6 with HCl (1 M, aq.), then extracted with EA (2×50 mL). The combined organic layers were washed with saturated brine (50 mL), and dried over anhydrous Na₂SO₄, and filtered. The filtrate was concentrated under reduced pressure. This provided 2-[3-[4-(tert-butoxycarbonyl)piperazin-1-yl]-1,2-oxazol-5-yl]-3-methylbutanoic acid (52 mg, crude) as a yellow solid. LCMS (ESI) m/z: [M+H]⁺=354.

### Step 4: Preparation of tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate

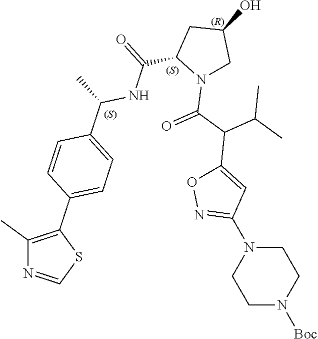

To a stirred solution of 2-[3-[4-(tert-butoxycarbonyl)piperazin-1-yl]-1,2-oxazol-5-yl]-3-methylbutanoic acid (52.00 mg, 0.119 mmol, 1.00 equiv) in DMF (2.00 mL) was added HATU (135.56 mg, 0.357 mmol, 3.00 equiv) and DIEA (76.80 mg, 0.595 mmol, 5.00 equiv) at room temperature. To the above mixture was added (2S,4R)-4-hydroxy-N-[(1S)-1-[4-(4-methyl-1,3-thiazol-5-yl)phenyl]ethyl]pyrrolidine-2-carboxamide (70.90 mg, 0.214 mmol, 1.80 equiv) at room temperature. The resulting mixture was stirred for an additional 1 h. The mixture was purified directly by reverse phase flash chromatography with the following conditions: column, C18 silica gel; mobile phase, MeCN in water (0.1% FA), 0 to 100% gradient in 30 min. This provided in tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate (73 mg, 92.12%) as a white solid. LCMS (ESI) m/z: [M+H]⁺=667.

### Step 5: Preparation of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide; (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide

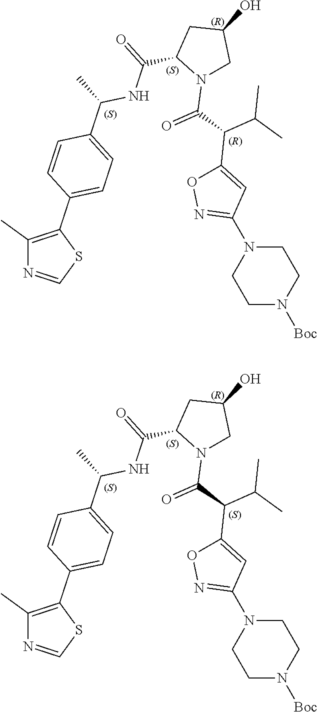

The tert-butyl 4-(5-[1-[(2S,4R)-4-hydroxy-2-[[(1S)-1-[4-(4-methyl-1,3-thiazol-5-yl)phenyl]ethyl]carbamoyl]pyrrolidin-1-yl]-3-methyl-1-oxobutan-2-yl]-1,2-oxazol-3-yl)piperazine-1-carboxylate (73 mg) was purified by SFC with the following conditions: Column, CHIRAL ART Amylose-C NEO, 3*25 cm, 5 um; mobile phase, MeOH. This provided (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (37 mg, second peak). LCMS (ESI) m/z: [M+H]⁺=667, and (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (34 mg, first peak). LCMS (ESI) m/z: [M+H]⁺=667.

### Step 6: Preparation of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (I-70) and (2S,4R)-4-hydroxy-N-[(1S)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (I-71)

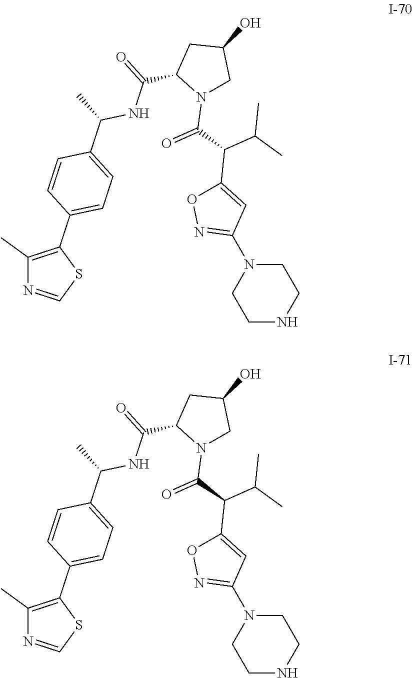 [SMILES: Cc1ncsc1-c1ccc([C@H](C)NC(=O)[C@@H]2C[C@@H](O)CN2C(=O)[C@@H](c2cc(N3CCNCC3)no2)C(C)C)cc1 ; Cc1ncsc1-c1ccc([C@H](C)NC(=O)[C@@H]2C[C@@H](O)CN2C(=O)[C@H](c2cc(N3CCNCC3)no2)C(C)C)cc1]

To a stirred solution of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (37.00 mg, 0.055 mmol, 1.00 equiv) in DCM (1.50 mL) was added HCl in 1,4-dioxane (1.50 mL, 26.276 mmol, 473.57 equiv) at 0° C. The resulting mixture was stirred for 1 h at room temperature, then concentrated under reduced pressure. This provided 1-70 (45 mg, crude) as a yellow oil. LCMS (ESI) m/z: [M+H]⁺=567. I-71 was prepared following the same protocol as 1-70 as a yellow oil. LCMS (ESI) m/z: [M+H]⁺=567.

The following intermediates in Table 7 were prepared in a similar manner as described in the preparation of intermediate I-70 starting with methyl 3-methyl-2-[3-[(1,1,2,2,3,3,4,4,4-nonafluorobutanesulfonyl)oxy]-1,2-oxazol-5-yl]butanoate and the appropriate amines.

TABLE 7

| | Intermediate | | LCMS |
|---|---|---|---|
| Structure | No. | Name | (ESI) m/z |
| 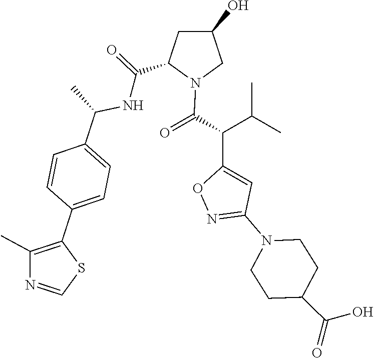 [SMILES: Cc1ncsc1-c1ccc([C@H](C)NC(=O)[C@@H]2C[C@@H](O)CN2C(=O)[C@@H](c2cc(N3CCC(C(=O)O)CC3)no2)C(C)C)cc1] | I-72 | 1-{5-[(2R)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl-1- oxobutan-2-yl]-1,2-oxazol-3- yl}piperidine-4-carboxylic acid | 610 |
| 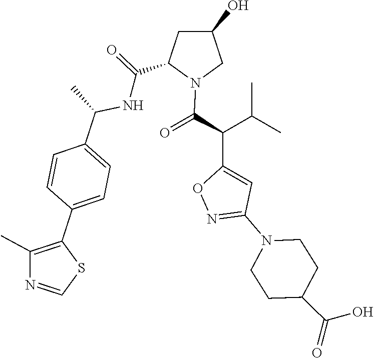 [SMILES: Cc1ncsc1-c1ccc([C@H](C)NC(=O)[C@@H]2C[C@@H](O)CN2C(=O)[C@H](c2cc(N3CCC(C(=O)O)CC3)no2)C(C)C)cc1] | I-73 | 1-{5-[(2S)-1-[(2S,4R)-4- hydroxy-2-{[(1S)-1-[4-(4- methyl-1,3-thiazol-5- yl)phenyl]ethyl]carbamoyl} pyrrolidin-1-yl]-3-methyl-1- oxobutan-2-yl]-1,2-oxazol-3- yl}piperidine-4-carboxylic acid | 610 |
| 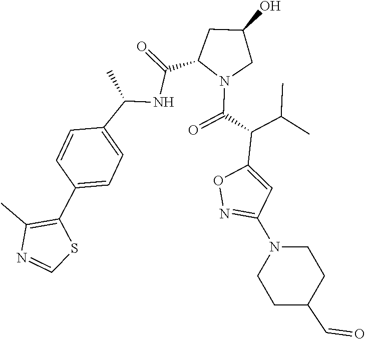 [SMILES: Cc1ncsc1-c1ccc([C@H](C)NC(=O)[C@@H]2C[C@@H](O)CN2C(=O)[C@@H](c2cc(N3CCC(C=O)CC3)no2)C(C)C)cc1] | I-74 | (2S,4R)-1-[(2R)-2-[3-(4- formylpiperidin-1-yl)-1,2- oxazol-5-yl]-3-methylbutanoyl]- 4-hydroxy-N-[(1S)-1-[4-(4- methyl-1,3-thiazol-5- yl)phenyl]ethyl]pyrrolidine-2- carboxamide | 594 |
| 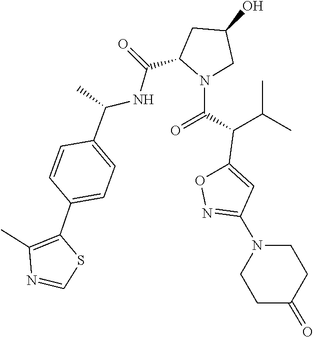 [SMILES: Cc1ncsc1-c1ccc([C@H](C)NC(=O)[C@@H]2C[C@@H](O)CN2C(=O)[C@@H](c2cc(N3CCC(=O)CC3)no2)C(C)C)cc1] | I-75 | (2S,4R)-4-hydroxy-1-((R)-3- methyl-2-(3-(4-oxopiperidin-1- yl)isoxazol-5-yl)butanoyl)-N- ((S)-1-(4-(4-methylthiazol-5- yl)phenyl)ethyl)pyrrolidine-2- carboxamide | 580 |
| 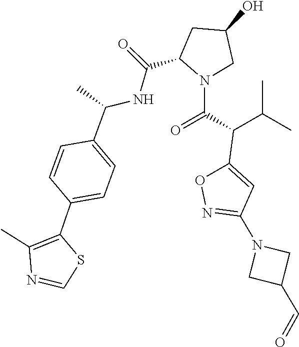 [SMILES: Cc1ncsc1-c1ccc([C@H](C)NC(=O)[C@@H]2C[C@@H](O)CN2C(=O)[C@@H](c2cc(N3CC(C=O)C3)no2)C(C)C)cc1] | I-76 | (2S,4R)-1-((R)-2-(3-(3- formylazetidin-1-yl)isoxazol-5- yl)-3-methylbutanoyl)-4- hydroxy-N-((S)-1-(4-(4- methylthiazol-5- yl)phenyl)ethyl)pyrrolidine-2- carboxamide | 566 |
| 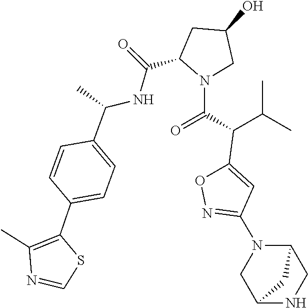 [SMILES: Cc1ncsc1-c1ccc([C@H](C)NC(=O)[C@@H]2C[C@@H](O)CN2C(=O)[C@@H](c2cc(N3C[C@H]4C[C@@H]3CN4)no2)C(C)C)cc1] | I-77 | (2S,4R)-1-[(2R)-2-{3-[(1R,4R)- 2,5-diazabicyclo[2.2.1]heptan- 2-yl]-1,2-oxazol-5-yl}-3- methylbutanoyl]-4-hydroxy-N- [(1S)-1-[4-(4-methyl-1,3- thiazol-5- yl)phenyl]ethyl]pyrrolidine-2- carboxamide | 579 |
| 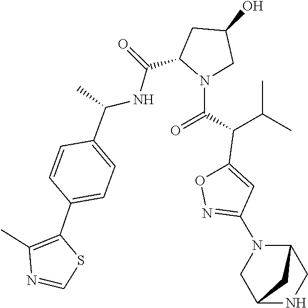 [SMILES: Cc1ncsc1-c1ccc([C@H](C)NC(=O)[C@@H]2C[C@@H](O)CN2C(=O)[C@@H](c2cc(N3C[C@@H]4C[C@H]3CN4)no2)C(C)C)cc1] | I-78 | (2S,4R)-1-((R)-2-(3-((1S,4S)- 2,5-diazabicyclo[2.2.1]heptan- 2-yl)isoxazol-5-yl)-3- methylbutanoyl)-4-hydroxy-N- ((S)-1-(4-(4-methylthiazol-5- yl)phenyl)ethyl)pyrrolidine-2- carboxamide | 579 |
| 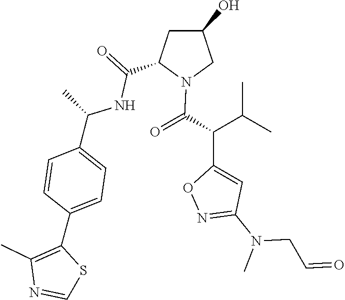 [SMILES: Cc1ncsc1-c1ccc([C@H](C)NC(=O)[C@@H]2C[C@@H](O)CN2C(=O)[C@@H](c2cc(N(C)CC=O)no2)C(C)C)cc1] | I-79 | (2S,4R)-4-hydroxy-1-((R)-3- methyl-2-(3-(methyl(2- oxoethyl)amino)isoxazol-5- yl)butanoyl)-N-((S)-1-(4-(4- methylthiazol-5- yl)phenyl)ethyl)pyrrolidine-2- carboxamide | 554 |

### Preparation of tert-butyl (2S,4R)-4-hydroxy-N-[(1S)-1-[4-(4-methyl-1,3-thiazol-5-yl)phenyl]ethyl]-1-(2R)-3-methyl-2-[3-(piperidin-4-yl)-1,2-oxazol-5-yl]butanoyl]pyrrolidine-2-carboxamide (I-80)

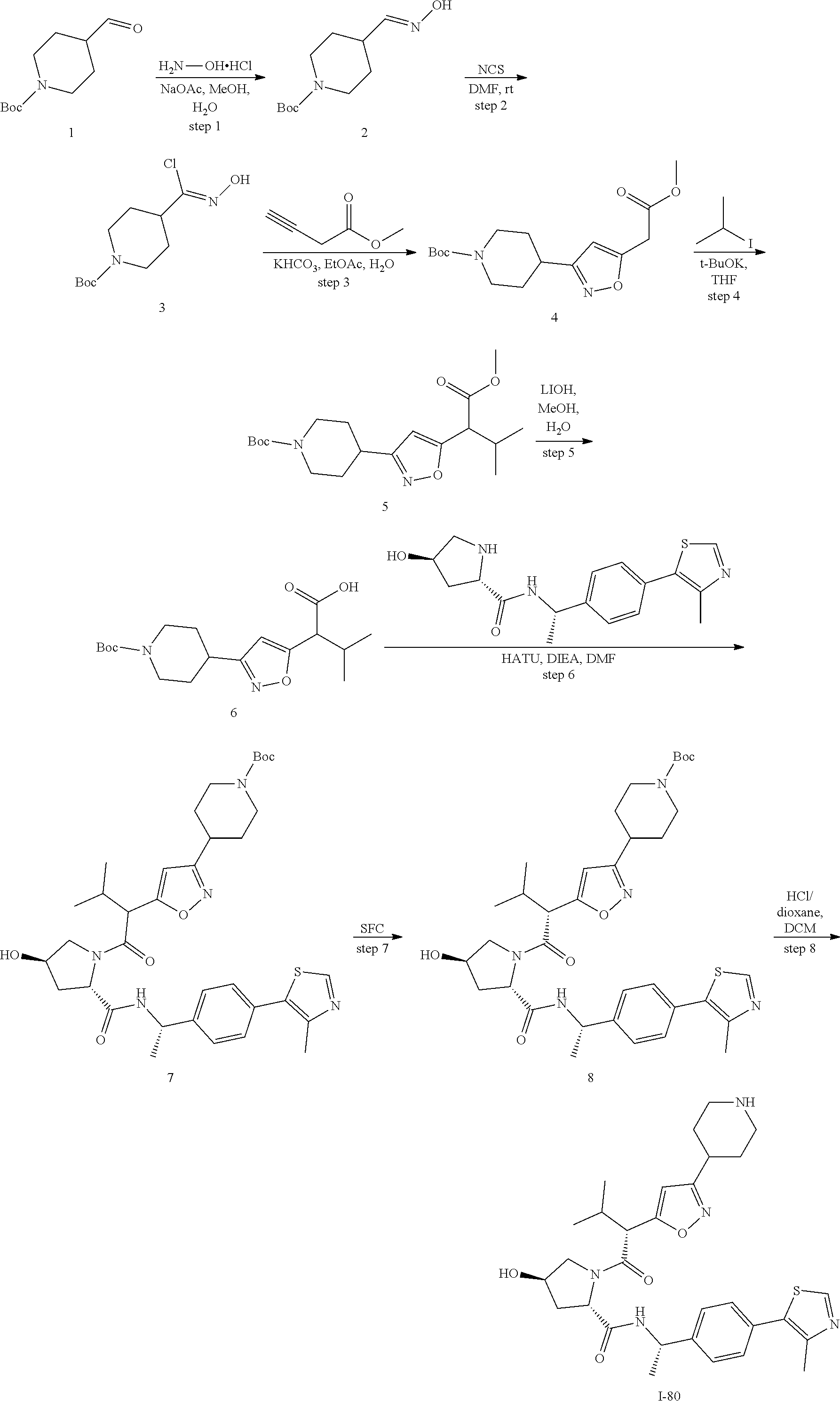

### Step 1: Preparation of tert-butyl 4-[(1E)-(hydroxyimino)methyl]piperidine-1-carboxylate (Intermediate 2)

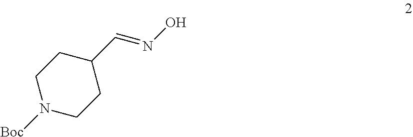 [SMILES: CC(C)(C)OC(=O)N1CCC(/C=N/O)CC1]

To a stirred solution of tert-butyl 4-formylpiperidine-1-carboxylate (5 g, 23.4 mmol, 1.00 equiv) in MeOH (10 mL) and H₂O (10 mL) was added hydroxylamine hydrochloride (1.95 g, 28.133 mmol, 1.2 equiv) and Na₂CO₃ (1.24 g, 11.722 mmol, 0.5 equiv) at 0 degrees C. The resulting mixture was stirred overnight at room temperature. The resulting mixture was concentrated under reduced pressure. The resulting mixture was extracted with EtOAc (3×20 mL). The combined organic layers were washed with brine (50 mL), and dried over anhydrous Na₂SO₄. After filtration, the filtrate was concentrated under reduced pressure to afford intermediate 2 (6 g, crude) as a colorless oil. LCMS (ESI) m/z: [M+H]⁺=229.

### Step 2: Preparation of tert-butyl 4-[(1Z)-chloro(hydroxyimino)methyl]piperidine-1-carboxylate (Intermediate 3)

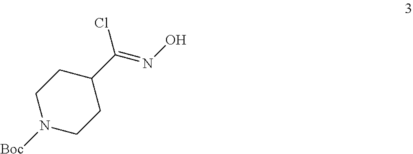 [SMILES: CC(C)(C)OC(=O)N1CCC(/C(Cl)=N/O)CC1]

A mixture of intermediate 2 and NCS (3.5 g, 26.282 mmol, 1.0 equiv) in DMF (20 mL) was stirred for 2 h at room temperature. The desired product could be detected by LCMS. The resulting mixture was diluted with water (50.00 mL) and extracted with EtOAc (3×50 mL). The combined organic layers were washed with brine (50 mL), and dried over anhydrous Na₂SO₄. After filtration, the filtrate was concentrated under reduced pressure to afford intermediate 3 (7.8 g, crude) as a colorless oil. LCMS (ESI) m/z [M+H]⁺=263.

### Step 3: Preparation of tert-butyl 4-[5-(2-methoxy-2-oxoethyl)-1,2-oxazol-3-yl]piperidine-1-carboxylate (Intermediate 4)

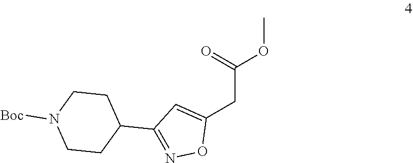 [SMILES: COC(=O)Cc1cc(C2CCN(C(=O)OC(C)(C)C)CC2)no1]

A mixture of intermediate 3 (7.8 g, crude) and NaHCO₃ (3.8 g, 45.675 mmol, 1.5 equiv) in EtOAc (100 mL) was stirred for 30 min at room temperature. To the above mixture was added methyl but-3-ynoate (2.99 g, 30.450 mmol, 1 equiv) at 0 degrees C. The resulting mixture was stirred overnight at room temperature. The desired product could be detected by LCMS. The resulting mixture was concentrated under reduced pressure. The residue was purified by reverse phase flash chromatography with the following conditions: column, C18 silica gel; mobile phase, MeCN in water (0.05% FA), 0% to 100% gradient in 30 min; detector, UV 254 nm. The resulting mixture was concentrated under reduced pressure to afford intermediate 4 (4.1 g, 41.51%) as a light yellow oil. LCMS (ESI) m/z: [M+H]⁺=325.

### Step 4: Preparation of tert-butyl 4-[5-(1-methoxy-3-methyl-1-oxobutan-2-yl)-1,2-oxazol-3-yl]piperidine-1-carboxylate (Intermediate 5)

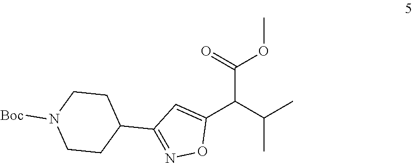 [SMILES: COC(=O)C(c1cc(C2CCN(C(=O)OC(C)(C)C)CC2)no1)C(C)C]

A mixture of intermediate 4 (1.0 g, 3.083 mmol, 1.5 equiv) and Na₂SO₄ (1.0 g) in THF (10 mL) was added t-BuOK (518.90 mg, 4.625 mmol, 1.5 equiv) and 2-iodopropane (628.87 mg, 3.700 mmol, 1.2 equiv) at 0 degrees C. under an atmosphere of dry nitrogen. The resulting mixture was stirred for 3 h at 0 degrees C. under an atmosphere of dry nitrogen. The desired product could be detected by LCMS. After filtration, the filtrate was concentrated under reduced pressure. The residue was purified by reverse phase flash chromatography with the following conditions: column, C18 silica gel; mobile phase, MeCN in water (0.05% FA), 0% to 100% gradient in 30 min; detector, UV 254 nm. The resulting mixture was concentrated under reduced pressure to afford intermediate 5 (330 mg, 29.21%) as a light yellow oil. LCMS (ESI) m/z: [M+H]⁺=367.

### Step 5: Preparation of 2-{3-[1-(tert-butoxycarbonyl)piperidin-4-yl]-1,2-oxazol-5-yl}-3-methylbutanoic acid (Intermediate 6)

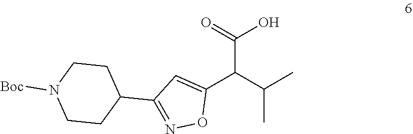 [SMILES: CC(C)C(C(=O)O)c1cc(C2CCN(C(=O)OC(C)(C)C)CC2)no1]

To a stirred solution of intermediate 5 (320 mg, 0.873 mmol, 1.00 equiv) in MeOH (5 mL) was added LiOH (62.74 mg, 2.619 mmol, 3 equiv) in H₂O (5 mL) dropwise at room temperature. The resulting mixture was stirred for 3 h at room temperature. The desired product could be detected by LCMS. The resulting mixture was concentrated under reduced pressure. To the above mixture was added aq. HCl (6M) adjusting pH to ˜5. The resulting mixture was extracted with EtOAc (3×50 mL). The combined organic layers were washed with brine (50 mL), and dried over anhydrous Na₂SO₄. After filtration, the filtrate was concentrated under reduced pressure to afford intermediate 6 (316 mg crude) as an off-white solid. LCMS (ESI) m/z: [M+H]⁺=353.

### Step 6: Preparation of tert-butyl 4-(5-{1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl}-1,2-oxazol-3-yl)piperidine-1-carboxylate (Intermediate 7)

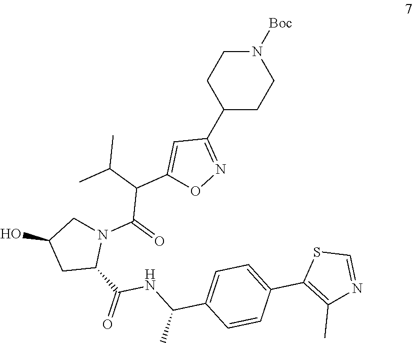 [SMILES: Cc1ncsc1-c1ccc([C@H](C)NC(=O)[C@@H]2C[C@@H](O)CN2C(=O)C(c2cc(C3CCN(C(=O)OC(C)(C)C)CC3)no2)C(C)C)cc1]

A mixture of intermediate 6 (310 mg, 0.880 mmol, 1.00 equiv) and HATU (668.90 mg, 1.760 mmol, 2 equiv) in DMF (5 mL) was stirred for 30 min at room temperature. To the above mixture was added (2S,4R)-4-hydroxy-N-[(1S)-1-[4-(4-methyl-1,3-thiazol-5-yl) phenyl]ethyl]pyrrolidine-2-carboxamide (291.53 mg, 0.880 mmol, 1 equiv) at room temperature. The resulting mixture was stirred for additional 2 h at room temperature. The desired product could be detected by LCMS. The residue was purified by reverse phase flash chromatography with the following conditions: column, C18 silica gel; mobile phase, MeCN in water (0.05% FA), 0% to 100% gradient in 30 min; detector, UV 254 nm. The resulting mixture was concentrated under reduced pressure to afford intermediate 7 (242 mg, 37.31%) as a light brown solid. LCMS (ESI) m/z: [M+H]⁺=666.

### Step 7: Preparation of tert-butyl 4-{5-[(2R)-1-[(2S,4R)-4-hydroxy-2-{[(1S)-1-[4-(4-methyl-1,3-thiazol-5-yl)phenyl]ethyl]carbamoyl}pyrrolidin-1-yl]-3-methyl-1-oxobutan-2-yl]-1,2-oxazol-3-yl}piperidine-1-carboxylate (Intermediate 8)

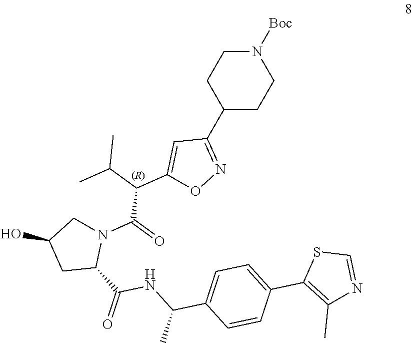

Intermediate 7 was purified by Prep-SFC with the following conditions (Column: CHIRAL ART Amylose-SA, 3*25 cm, 5 μm; Mobile Phase A: CO2, Mobile Phase B: MeOH—HPLC; Flow rate: 50 mL/min; Gradient: isocratic 45% B; Column Temperature (° C.): 35; Back Pressure(bar): 100; Wave Length: 205 nm; RT1(min): 3.65; RT2(min): 4.88; Sample Solvent: MeOH—HPLC; Injection Volume: 1 mL) to afford intermediate 8 (the second peak) (208.1 mg, 43.52%) as a light brown solid. LCMS (ESI) m/z: [M+H]⁺=666.

### Step 8: Preparation of tert-butyl (2S,4R)-4-hydroxy-N-[(1S)-1-[4-(4-methyl-1,3-thiazol-5-yl)phenyl]ethyl]-1-[(2R)-3-methyl-2-[3-(piperidin-4-yl)-1,2-oxazol-5-yl]butanoyl]pyrrolidine-2-carboxamide (I-80)

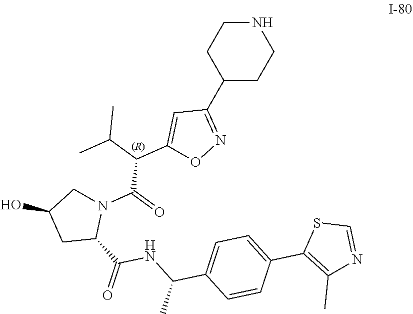

To a stirred solution of intermediate 8 (200 mg, 0.300 mmol, 1.00 equiv) in CM (2 mL) was added 1 M HCl in 1,4-dioxane (2 mL) dropwise at room temperature. The resulting mixture was stirred for 1 h at room temperature. The desired product could be detected by LCMS. The resulting mixture was concentrated under reduced pressure to afford I-80 (247.5 mg) as a light yellow solid. LCMS (ESI) m/z: [M+H]⁻=566.

The following intermediates in Table 8 were prepared in a similar manner as described in the preparation of intermediate I-80 starting with appropriate aldehyde.

TABLE 8

| | Intermediate | | LCMS |
|---|---|---|---|
| Structure | No. | Name | (ESI) m/z |
| 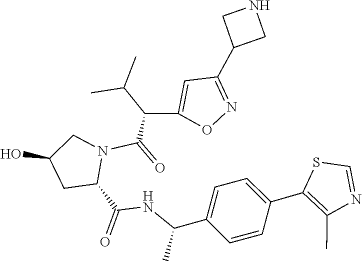 [SMILES: Cc1ncsc1-c1ccc([C@H](C)NC(=O)[C@@H]2C[C@@H](O)CN2C(=O)[C@@H](c2cc(C3CNC3)no2)C(C)C)cc1] | I-81 | (2S,4R)-1-((R)-2-(3-(azetidin- 3-yl)isoxazol-5-yl)-3- methylbutanoyl)-4-hydroxy-N- ((S)-1-(4-(4-methylthiazol-5- yl)phenyl)ethyl)pyrrolidine-2- carboxamide | 538 |
| 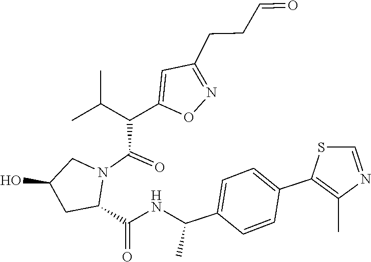 | I-82 | (2S,4R)-4-hydroxy-1-((R)-3- methyl-2-(3-(3- oxopropyl)isoxazol-5- yl)butanoyl)-N-((S)-1-(4-(4- methylthiazol-5- yl)phenyl)ethyl)pyrrolidine-2- carboxamide | 539 |
| 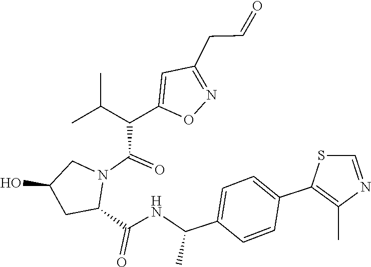 | I-83 | (2S,4R)-4-hydroxy-N-[(1S)-1- [4-(4-methyl-1,3-thiazol-5- yl)phenyl]ethyl]-1-{3-methyl-2- [3-(2-oxoethyl)-1,2-oxazol-5- yl]butanoyl}pyrrolidine-2- carboxamide | 525 |
| 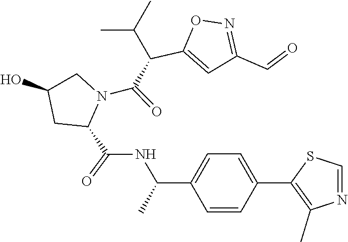 [SMILES: Cc1ncsc1-c1ccc([C@H](C)NC(=O)[C@@H]2C[C@@H](O)CN2C(=O)[C@@H](c2cc(C=O)no2)C(C)C)cc1] | I-84 | (2S,4R)-1-[(2R)-2-(3-formyl- 1,2-oxazol-5-yl)-3- methylbutanoyl]-4-hydroxy-N- [(1S)-1-[4-(4-methyl-1,3- thiazol-5- yl) phenyl]ethyl]pyrrolidine-2- carboxamide | 511 |

### Preparation of 3-(3-(2-hydroxyphenyl)-7H-pyrrolo[2,3-c]pyridazin-6-yl)bicyclo[1.1.1] pentane-1-carbaldehyde (I-87)

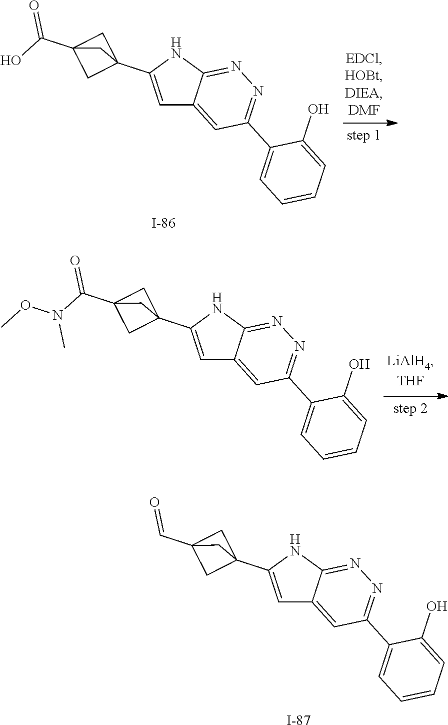

### Step 1: Preparation of 3-(3-(2-hydroxyphenyl)-7H-pyrrolo[2,3-c]pyridazin-6-yl)-N-methoxy-N-methylbicyclo[1.1.1]pentane-1-carboxamide

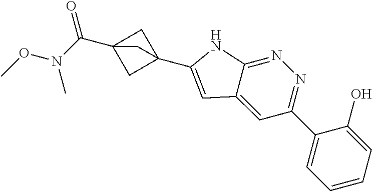 [SMILES: CON(C)C(=O)C12CC(c3cc4cc(-c5ccccc5O)nnc4[nH]3)(C1)C2]

To a stirred mixture of 3-[3-(2-hydroxyphenyl)-7H-pyrrolo[2,3-c]pyridazin-6-yl]bicyclo[1.1.1]pentane-1-carboxylic acid (30 mg, 0.093 mmol, 1.00 equiv) in DMF (1.0 mL) were added HOBT (18.92 mg, 0.140 mmol, 1.50 equiv) and EDCI (26.85 mg, 0.140 mmol, 1.5 equiv) at room temperature. After 10 min, to the above mixture was added DIEA (60.33 mg, 0.465 mmol, 5.0 equiv) and N,O-dimethylhydroxylamine hydrochloride (27.32 mg, 0.279 mmol, 3.0 equiv) at room temperature. The resulting mixture was stirred for additional 2 h at room temperature. The residue was purified by reverse phase flash chromatography with the following conditions: column, silica gel; mobile phase, MeCN in water (0.1% FA), 0% to 50% gradient in 40 min; detector, UV 254 nm. This resulted in the title compound (20 mg, 52.91%) as a yellow solid. LCMS (ESI) m/z: [M+H]⁺=306.

### Step 2: Preparation of 3-(3-(2-hydroxyphenyl)-7H-pyrrolo[2,3-c]pyridazin-6-yl)bicyclo[1.1.1] pentane-1-carbaldehyde (I-87)

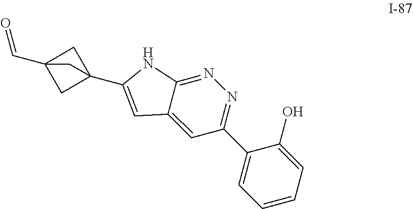 [SMILES: O=CC12CC(c3cc4cc(-c5ccccc5O)nnc4[nH]3)(C1)C2]

To a stirred solution of 3-(3-(2-hydroxyphenyl)-7H-pyrrolo[2,3-c]pyridazin-6-yl)-N-methoxy-N-methylbicyclo[1.1.1]pentane-1-carboxamide (30 mg, 0.082 mmol, 1.00 equiv) in THF (1 mL) was added LiAlH₄ (3.12 mg, 0.082 mmol, 1 equiv), and the mixture was stirred at 0 degrees C. for 2 h. The resulting mixture was concentrated under reduced pressure. The residue was purified by reverse phase flash chromatography with the following conditions: column, C18 silica gel; mobile phase, CH₃CN in water (0.05% TFA), 10% to 50% gradient in 30 min; detector, UV 254 nm to yield 1-87 (22 mg, 87.52%) as a yellow solid. LCMS (ESI) m/z: [M+H]⁺=306.

### Preparation of 3-([3-[3-(2-hydroxyphenyl)-7H-pyrrolo[2,3-c]pyridazin-6-yl]bicyclo[1.1.1]pentan-1-yl]amino)propanoic acid (I-88)

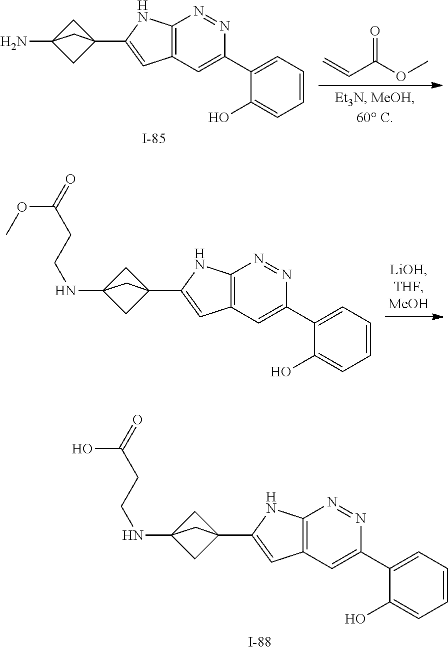

### Step 1: Preparation of methyl 3-((3-(3-(2-hydroxyphenyl)-7H-pyrrolo[2,3-c]pyridazin-6-yl)bicyclo[1.1.1]pentan-1-yl)amino)propanoate

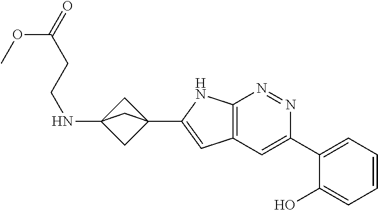 [SMILES: COC(=O)CCNC12CC(c3cc4cc(-c5ccccc5O)nnc4[nH]3)(C1)C2]

A solution of 2-(6-[3-aminobicyclo[1.1.1]pentan-1-yl]-7H-pyrrolo[2,3-c]pyridazin-3-yl)phenol (100.00 mg, 0.342 mmol, 1.00 equiv), methyl acrylate (23.56 mg, 0.274 mmol, 0.80 equiv) and trimethylamine (103.84 mg, 1.026 mmol, 3 equiv) in methanol (2.00 mL) was stirred overnight at 60 degrees C. Without any additional work-up, the residue was purified by reverse phase flash chromatography with the following conditions: column, C18 silica gel; mobile phase, CH₃CN in water (0.05% FA), 0% to 100% gradient in 25 min; detector, UV 254 nm. This resulted in methyl 3-((3-(3-(2-hydroxyphenyl)-7H-pyrrolo[2,3-c]pyridazin-6-yl)bicyclo[1.1.1]pentan-1-yl)amino)propanoate (39 mg, 30.1%) as a black oil. LCMS (ESI) m/z: [M+H]⁺=379.

### Step 2: Preparation of 3-([3-[3-(2-hydroxyphenyl)-7H-pyrrolo[2,3-c]pyridazin-6-yl]bicyclo[1.1.1]pentan-1-yl]amino)propanoic acid (I-88)

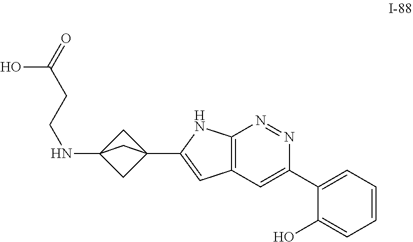 [SMILES: O=C(O)CCNC12CC(c3cc4cc(-c5ccccc5O)nnc4[nH]3)(C1)C2]

A solution of methyl 3-((3-(3-(2-hydroxyphenyl)-7H-pyrrolo[2,3-c]pyridazin-6-yl)bicyclo[1.1.1]pentan-1-yl)amino)propanoate (39.00 mg, 0.103 mmol, 1.00 equiv) and LiOH (24.68 mg, 1.031 mmol, 10.00 equiv) in THF (0.90 mL), H₂O (0.30 mL) was stirred for 2 h at room temperature. The reaction was monitored by LCMS. The mixture was acidified to pH 5 with conc. hydrochloric acid. The resulting mixture was extracted with EtOAc (3×50 mL). The combined organic layers were washed with brine (100 mL), and dried over anhydrous Na₂SO₄. After filtration, the filtrate was concentrated under reduced pressure. This resulted in I-88 (22 mg, 58.58%) as a yellow oil. LCMS (ESI) m/z: [M+H]⁺=365.

### Preparation of 3-(2-hydroxyphenyl)-7H-pyrrolo[2,3-c]pyridazine-6-carboxylic acid (I-89)

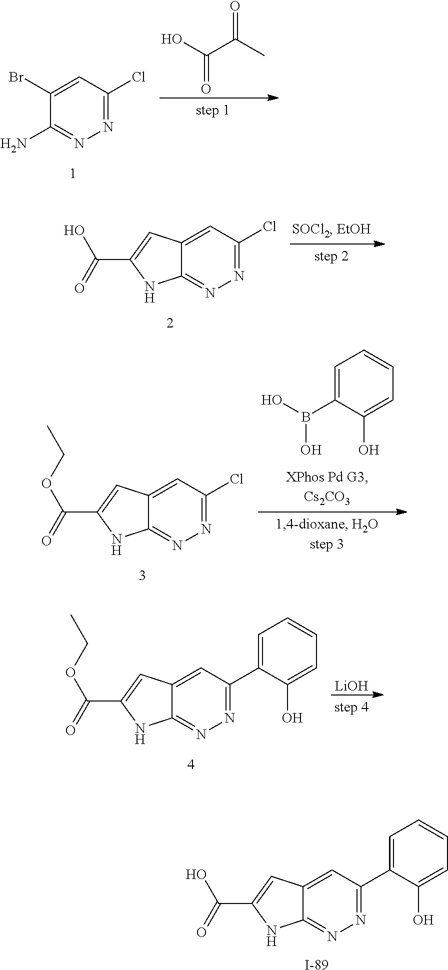

### Step 1: Preparation of 3-chloro-7H-pyrrolo[2,3-c]pyridazine-6-carboxylic acid (Intermediate 2)

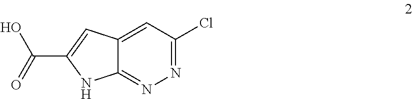 [SMILES: O=C(O)c1cc2cc(Cl)nnc2[nH]1]

To a solution of 4-bromo-6-chloropyridazin-3-amine (500.00 mg, 2.399 mmol, 1.00 equiv) and pyruvic acid (633.72 mg, 7.196 mmol, 3.00 equiv) in DMF (8.00 mL) was added Pd(OAc)₂ (53.85 mg, 0.240 mmol, 0.10 equiv), DABCO (805.99 mg, 7.196 mmol, 3.00 equiv) and MgSO₄ (250.00 mg, 2.077 mmol, 0.87 equiv). The reaction mixture was stirred for 6 h at 105 degrees C. The residue was purified by reverse phase flash chromatography with the following conditions: column, C18 silica gel; mobile phase, CH₃CN in water, 0% to 50% gradient in 20 min; detector, UV 254 nm. This resulted in intermediate 2 (70 mg, 14.77%) as a brown solid. LCMS (ESI) m/z: [M+H]⁺=198.

### Step 2: Preparation of ethyl 3-chloro-7H-pyrrolo[2,3-c]pyridazine-6-carboxylate (Intermediate 3)

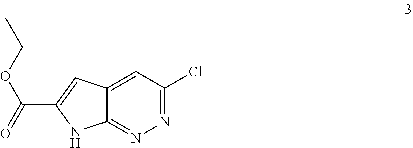

To a solution of intermediate 2 (2 g, 10.122 mmol, 1 equiv) in EtOH (15 mL) was added SOCl₂ (1.81 g, 15.183 mmol, 1.5 equiv). The resulting mixture was stirred for 4 h at 50° C. under an atmosphere of dry nitrogen. The resulting mixture was concentrated under vacuum. The residue was purified by silica gel column chromatography, eluted with PE/EA (1:1) to afford intermediate 3 (1 g, 43.78%) as a yellow oil. LCMS (ESI) m/z: [M+H]⁺=226.

### Step 3: Preparation of ethyl 3-(2-hydroxyphenyl)-7H-pyrrolo[2,3-c]pyridazine-6-carboxylate (Intermediate 4)

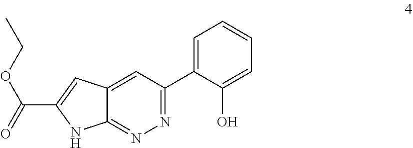 [SMILES: CCOC(=O)c1cc2cc(-c3ccccc3O)nnc2[nH]1]

To a solution of intermediate 3 (1 g, 4.432 mmol, 1.00 equiv) and 2-hydroxyphenylboronic acid (1.22 g, 8.864 mmol, 2 equiv) in dioxane (10 mL) and H₂O (2 mL) was added Cs₂CO₃ (4.33 g, 13.296 mmol, 3 equiv) and XPhos Pd G3 (0.38 g, 0.443 mmol, 0.1 equiv). The resulting mixture was stirred for 2 h at 80° C. under an atmosphere of dry nitrogen. This resulted in intermediate 4 (700 mg, 55.75%) as a brown oil. LCMS (ESI) m/z: [M+H]⁺=284.

### Step 4: Preparation of 3-(2-hydroxyphenyl)-7H-pyrrolo[2,3-c]pyridazine-6-carboxylic acid (I-89)

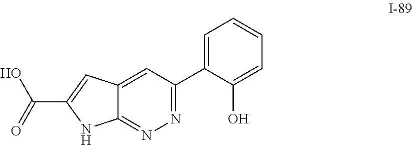 [SMILES: O=C(O)c1cc2cc(-c3ccccc3O)nnc2[nH]1]

To a solution of intermediate 4 (50 mg, 0.176 mmol, 1.00 equiv) in MeOH (2 mL) was added NaOH aq. solution (10 M, 0.2 mL). The reaction mixture was stirred for 2 h at 60 degrees C. The reaction mixture was concentrated under vacuum. The crude product was purified by Prep-HPLC with the following conditions: Column, XBridge Shield RP18 OBD Column, 19*150 mm, 5 μm; mobile phase, water (0.05% FA) and CH₃CN (15% CH₃CN up to 50% in 7 min); Detector, UV 254 nm. This resulted in I-89 (16.2 mg, 35.96%) as a green solid. ¹H NMR (400 MHz, DMSO-d6) δ 13.27 (d, J=177.8 Hz, 2H), 8.75 (s, 1H), 8.02 (dd, J=8.0, 1.6 Hz, 1H), 7.36-7.26 (m, 1H), 7.13-6.92 (m, 3H). LCMS (ESI) m/z: [M+H]⁺=256.05.

### Preparation of tert-butyl 4-(3-(2-hydroxyphenyl)-7H-pyrrolo[2,3-c]pyridazine-6-carbonyl)piperazine-1-carboxylate (I-90)

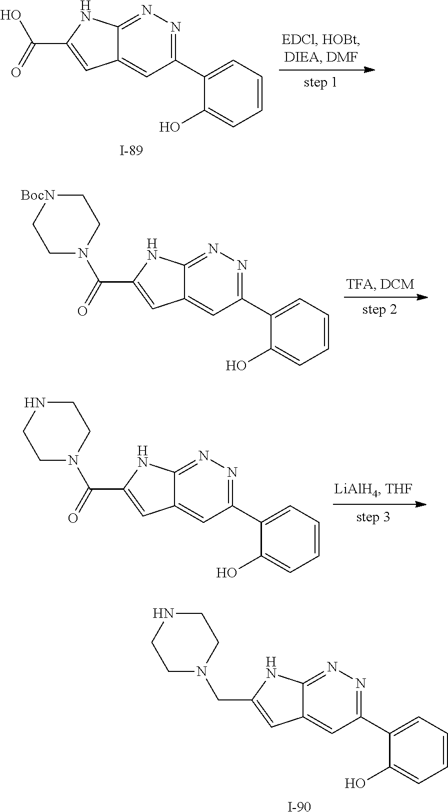

### Step 1: Preparation of tert-butyl 4-(3-(2-hydroxyphenyl)-7H-pyrrolo[2,3-c]pyridazine-6-carbonyl)piperazine-1-carboxylate

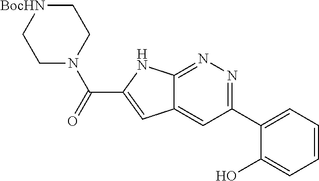

To a stirred solution of 3-(2-hydroxyphenyl)-7H-pyrrolo[2,3-c]pyridazine-6-carboxylic acid (60 mg, 0.235 mmol, 1.00 equiv) and tert-butyl piperazine-1-carboxylate (87.57 mg, 0.470 mmol, 2.0 equiv) in DMF (1 mL) was added EDCI (90.13 mg, 0.470 mmol, 2.0 equiv), HOBt (63.53 mg, 0.470 mmol, 2.0 equiv) and DIEA (151.91 mg, 1.175 mmol, 5.0 equiv) at room temperature. The resulting mixture was stirred for 12 h at room temperature. The residue was purified by reverse phase flash chromatography with the following conditions: column, C18 silica gel; mobile phase, CH₃CN in water (0.1% FA), 0% to 100% gradient in 25 min; Dector, UV 254 nm. This resulted in tert-butyl 4-(3-(2-hydroxyphenyl)-7H-pyrrolo[2,3-c]pyridazine-6-carbonyl)piperazine-1-carboxylate (70 mg, 70.32%) as an off-white solid. LCMS (ESI) m/z [M+H]⁺=424.

### Step 2: Preparation of 2-[6-(piperazine-1-carbonyl)-7H-pyrrolo[2,3-c]pyridazin-3-yl]phenol

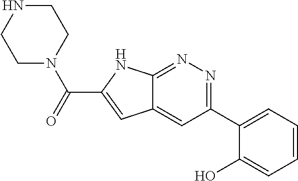 [SMILES: O=C(c1cc2cc(-c3ccccc3O)nnc2[nH]1)N1CCNCC1]

To a stirred solution of tert-butyl 4-(3-(2-hydroxyphenyl)-7H-pyrrolo[2,3-c]pyridazine-6-carbonyl)piperazine-1-carboxylate (70 mg, 0.165 mmol, 1.00 equiv) in DCM (5 mL) was added TFA (1 mL) dropwise at room temperature. The resulting mixture was stirred for 2 h at room temperature. The resulting mixture was concentrated under vacuum. This resulted in 2-[6-(piperazine-1-carbonyl)-7H-pyrrolo[2,3-c]pyridazin-3-yl]phenol (80 mg, crude) as a light yellow oil. LCMS (ESI) m/z [M+H]⁺=324.

### Step 3: Preparation of tert-butyl 4-(3-(2-hydroxyphenyl)-7H-pyrrolo[2,3-c]pyridazine-6-carbonyl)piperazine-1-carboxylate (I-90)

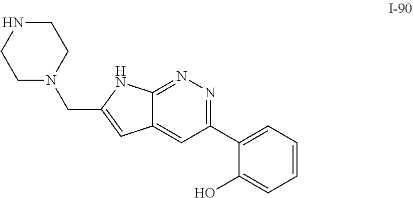 [SMILES: Oc1ccccc1-c1cc2cc(CN3CCNCC3)[nH]c2nn1]

To a stirred solution of 2-[6-(piperazine-1-carbonyl)-7H-pyrrolo[2,3-c]pyridazin-3-yl]phenol (70 mg, 0.216 mmol, 1.00 equiv) in THF (5 mL) was added LiAlH₄ (16.43 mg, 0.432 mmol, 2.0 equiv) in THF (1 mL) dropwise at 0 degrees C. under an atmosphere of dry nitrogen. The resulting mixture was stirred for 4 h at room temperature under an atmosphere of dry nitrogen. The reaction was quenched by the addition of water (5 mL) at room temperature. The resulting mixture was concentrated under reduced pressure. The residue was purified by reverse phase flash chromatography with the following conditions: column, C18 silica gel; mobile phase, CH₃CN in water (0.05% FA), 0% to 100% gradient in 30 min; detector, UV 254 nm. This resulted in I-90 (34 mg, 35.5%) as a light yellow solid.

### Preparation of rel-(R)-3-(2-hydroxyphenyl)-6,7,8,9-tetrahydro-5H-pyridazino[3,4-b]indole-6-carboxylic acid (I-91) and rel-(S)-3-(2-hydroxyphenyl)-6,7,8,9-tetrahydro-5H-pyridazino[3,4-b]indole-6-carboxylic acid (I-92)

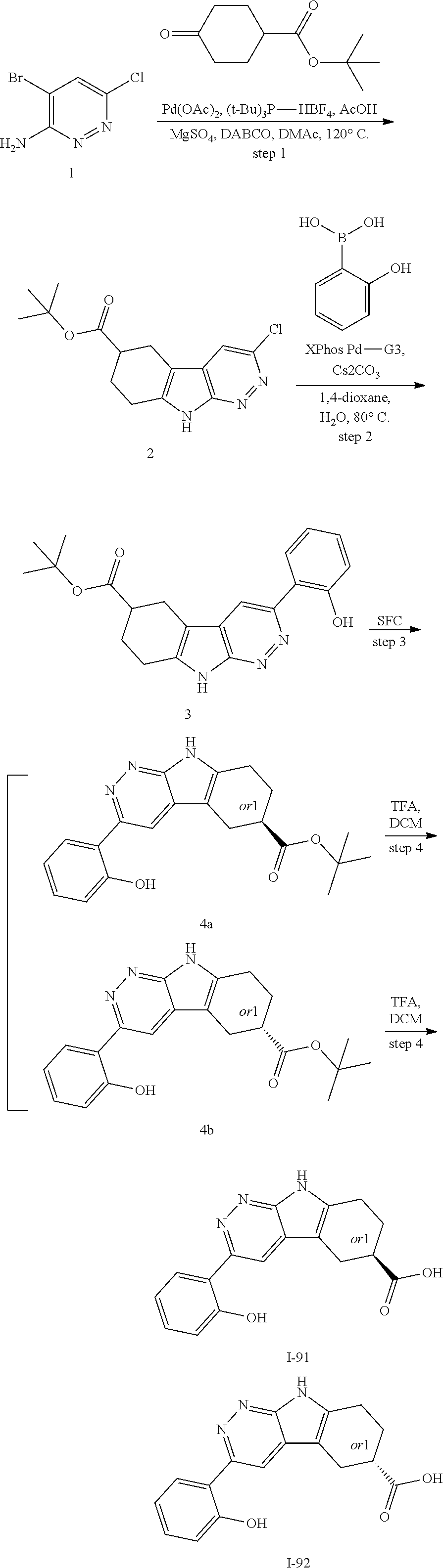

### Step 1: Preparation of tert-butyl 3-chloro-6,7,8,9-tetrahydro-5H-pyridazino[3,4-b]indole-6-carboxylate (intermediate 2)

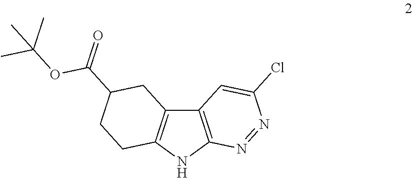 [SMILES: CC(C)(C)OC(=O)C1CCc2[nH]c3nnc(Cl)cc3c2C1]

To a stirred mixture of 4-bromo-6-chloropyridazin-3-amine (5256.68 mg, 25.219 mmol, 1.00 equiv) and tert-butyl 4-oxocyclohexane-1-carboxylate (5.00 g, 25.219 mmol, 1.00 equiv) in DMAc (80.00 mL) was added Pd(OAc)₂ (1132.39 mg, 5.044 mmol, 0.20 equiv), (t-Bu)3P-HBF4 (463.37 mg, 5.044 mmol, 0.2 equiv), AcOH (3028.92 mg, 50.438 mmol, 2.00 equiv), MgSO4 (5.00 mg, 0.042 mmol, 0.82 equiv) and DABCO (8483.47 mg, 75.657 mmol, 3.0 equiv) at room temperature under an atmosphere of dry nitrogen. The resulting mixture was stirred overnight at 120 degrees C. under an atmosphere of dry nitrogen. The mixture was allowed to cool down to room temperature. The resulting mixture was filtered, the filter cake was washed with EtOAc (2×10 mL). The filtrate was extracted with EtOAc (3×500 mL). The combined organic layers were washed with brine (100 mL), and dried over anhydrous Na2SO4. After filtration, the filtrate was concentrated under reduced pressure. The residue was purified by reverse phase flash chromatography with the following conditions: column, silica gel; mobile phase, MeCN in water, 0% to 100% gradient in 10 min; detector, UV 254 nm to afford intermediate 2 (350 mg, 4.28%) as a yellow solid. LCMS (ESI) m/z: [M+H]⁺=307.78.

### Step 2: Preparation of tert-butyl 3-(2-hydroxyphenyl)-6,7,8,9-tetrahydro-5H-pyridazino[3,4-b]indole-6-carboxylate (intermediate 3)

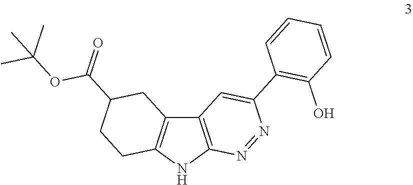 [SMILES: CC(C)(C)OC(=O)C1CCc2[nH]c3nnc(-c4ccccc4O)cc3c2C1]

To a solution of intermediate 2 (610 mg, 1.982 mmol, 1.00 equiv) and 2-hydroxyphenylboronic acid (410.05 mg, 2.973 mmol, 1.5 equiv) in 1,4-dioxane (10.00 mL) and H₂O (2.00 mL) were added Cs₂CO₃ (1291.51 mg, 3.964 mmol, 2.0 equiv) and XPhos Pd G3 (167.76 mg, 0.198 mmol, 0.1 equiv). The resulting mixture was stirred overnight at 80 degrees C. under an atmosphere of dry nitrogen. The resulting mixture was concentrated under reduced pressure. The residue was dissolved in DMF and purified by reverse phase flash chromatography with the following conditions: column, silica gel; mobile phase, MeCN in water, 0% to 100% gradient in 20 min; detector, UV 254 nm to afford intermediate 3 (450 mg, 59.65%) as a yellow solid. LCMS (ESI) m/z: [M+H]⁺=365.43.

### Step 3: Preparation of rel-tert-butyl (R)-3-(2-hydroxyphenyl)-6,7,8,9-tetrahydro-5H-pyridazino[3,4-b]indole-6-carboxylate (intermediate 4a) and (S)-3-(2-hydroxyphenyl)-6,7,8,9-tetrahydro-5H-pyridazino[3,4-b]indole-6-carboxylate (intermediate 4b)

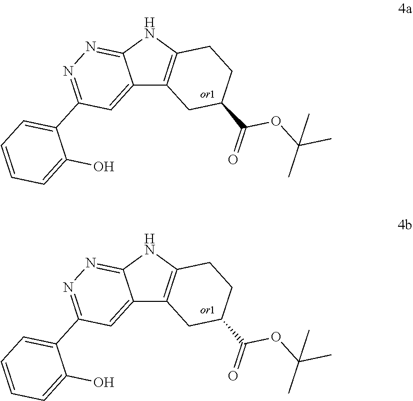 [SMILES: CC(C)(C)OC(=O)[C@@H]1CCc2[nH]c3nnc(-c4ccccc4O)cc3c2C1 ; CC(C)(C)OC(=O)[C@H]1CCc2[nH]c3nnc(-c4ccccc4O)cc3c2C1]

The intermediate 3 (680 mg) was purified by SFC with the following conditions: (Column: CHIRALPAK ID, 3×25 cm, 5 μm; Mobile Phase A: CO2, Mobile Phase B: EtOH—HPLC; Flow rate: 50 mL/min; Gradient: isocratic 55% B; Column Temperature (° C.): 35; Back Pressure(bar): 100; Wave Length: 217 nm; RT1(min): 5.1; RT2(min): 7.55; Sample Solvent: MeOH:DCM=1:1; Injection Volume: 8 mL; Number Of Runs: 9) to afford intermediate 4a (233 mg) as a brown solid and intermediate 4b (246 mg). LCMS (ESI) m/z: [M+H]⁺=365.43.

### Step 4: Preparation of rel-(R)-3-(2-hydroxyphenyl)-6,7,8,9-tetrahydro-5H-pyridazino[3,4-b]indole-6-carboxylic acid (I-91) and rel-(S)-3-(2-hydroxyphenyl)-6,7,8,9-tetrahydro-5H-pyridazino[3,4-b]indole-6-carboxylic acid (I-92)

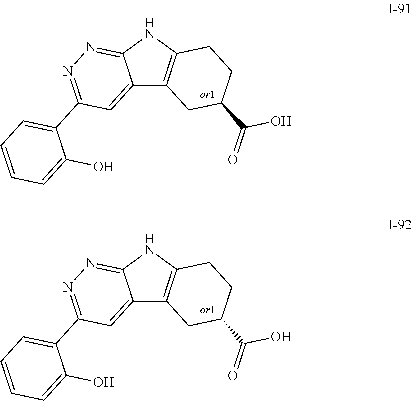 [SMILES: O=C(O)[C@@H]1CCc2[nH]c3nnc(-c4ccccc4O)cc3c2C1 ; O=C(O)[C@H]1CCc2[nH]c3nnc(-c4ccccc4O)cc3c2C1]

To a stirred solution of intermediate 4a (233.00 mg, 0.638 mmol, 1.00 equiv) in DCM (12.00 mL) was added TFA (4.00 mL, 53.852 mmol, 84.46 equiv) at room temperature. The resulting mixture was stirred for 4 h at room temperature. The resulting mixture was concentrated under reduced pressure. The residue was dissolved in ACN and water (50.00 ml) and lyophilized to provide 1-91 (170 mg, 79.04%) as a yellow solid. LCMS (ESI) m/z: [M+H]⁺=309.33. I-92 (190 mg, 89.42%) was afforded as a yellow solid using the same protocol starting from intermediate 4b. LCMS (ESI) m/z: [M+H]⁺=309.33.

### Preparation of (R)-2-(6-amino-6,7,8,9-tetrahydro-5H-pyridazino[3,4-b]indol-3-yl)phenol and (S)-2-(6-amino-6,7,8,9-tetrahydro-5H-pyridazino[3,4-b]indol-3-yl)phenol (I-93 and I-94)

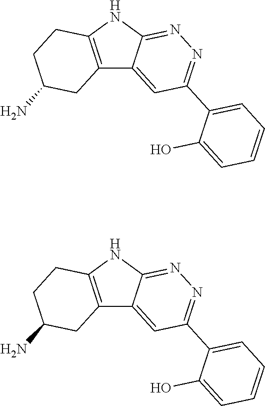 [SMILES: N[C@@H]1CCc2[nH]c3nnc(-c4ccccc4O)cc3c2C1 ; N[C@H]1CCc2[nH]c3nnc(-c4ccccc4O)cc3c2C1]

I-93 and I-94 were prepared as a yellow solid following the similar procedure as I-91 and I-92 starting from 4-bromo-6-chloropyridazin-3-amine and tert-butyl N-(4-oxocyclohexyl)carbamate. LCMS (ESI) m/z: [M+H]⁺=281.

### Preparation of (S)-2-(6-(methylamino)-6,7,8,9-tetrahydro-5H-pyridazino[3,4-b]indol-3-yl)phenol (I-95)

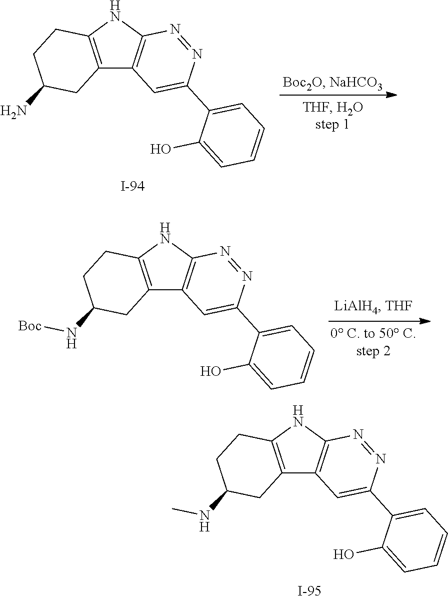

### Step 1: Preparation of tert-butyl (S)-(3-(2-hydroxyphenyl)-6,7,8,9-tetrahydro-5H-pyridazino[3,4-b]indol-6-yl)carbamate

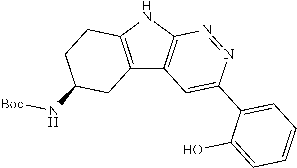 [SMILES: CC(C)(C)OC(=O)N[C@H]1CCc2[nH]c3nnc(-c4ccccc4O)cc3c2C1]

A mixture of 2-[(6S)-6-amino-5H,6H,7H,8H,9H-pyridazino[3,4-b]indol-3-yl]phenol (50.0 mg, 0.178 mmol, 1.00 equiv), (Boc)₂O (38.9 mg, 0.178 mmol, 1.00 equiv) and NaHCO₃ (29.9 mg, 0.356 mmol, 2.00 equiv) in THF (1 mL) and H₂O (1 mL) was stirred for 3 h at room temperature. The desired product could be detected by LCMS. The residue was purified by reverse phase flash chromatography with the following conditions: column, C18 silica gel; mobile phase, MeCN in Water (0.1% FA), 0% to 100% gradient in 25 min; detector, UV 254 and 220 nm. The resulting mixture was concentrated under reduced pressure to afford tert-butyl (S)-(3-(2-hydroxyphenyl)-6,7,8,9-tetrahydro-5H-pyridazino[3,4-b]indol-6-yl)carbamate (40 mg, 58.95%) as a yellow solid. LCMS (ESI) m/z: [M+H]⁺=381.

### Step 2: Preparation of (S)-2-(6-(methylamino)-6,7,8,9-tetrahydro-5H-pyridazino[3,4-b]indol-3-yl)phenol (I-95)

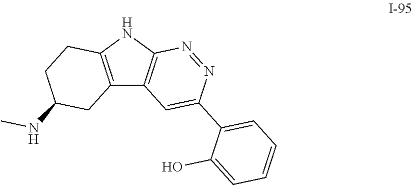 [SMILES: CN[C@H]1CCc2[nH]c3nnc(-c4ccccc4O)cc3c2C1]

A mixture of tert-butyl (S)-(3-(2-hydroxyphenyl)-6,7,8,9-tetrahydro-5H-pyridazino[3,4-b]indol-6-yl)carbamate (40.0 mg, 0.105 mmol, 1.00 equiv) and LiAlH₄ (7.9 mg, 0.210 mmol, 2.00 equiv) in THF (2 mL) was stirred for 5 min at 0° C. under an atmosphere of dry nitrogen. The resulting mixture was stirred for 1 h at 0° C. under an atmosphere of dry nitrogen and allowed to warm to RT and stirred for 2 h. The desired product could be detected by LCMS. The reaction was quenched by the addition of Na₂SO₄·10H₂O at 0° C. The precipitated solids were collected by filtration and washed with MeCN (3×30 mL). The resulting mixture was concentrated under reduced pressure. The residue was purified by reverse phase flash chromatography with the following conditions: column, C18 silica gel; mobile phase, MeCN in Water (0.1% FA), 0% to 100% gradient in 20 min; detector, UV 254 and 220 nm. The resulting mixture was concentrated under reduced pressure to afford I-95 (15 mg, 48.47%) as an off-white solid. LCMS (ESI) m/z: [M+H]⁺=295.

### Preparation of 2-{5′,7′-dihydrospiro[azetidine-3,6′-pyrrolo[2,3-c] pyridazin]-3′-yl}phenol (I-96)

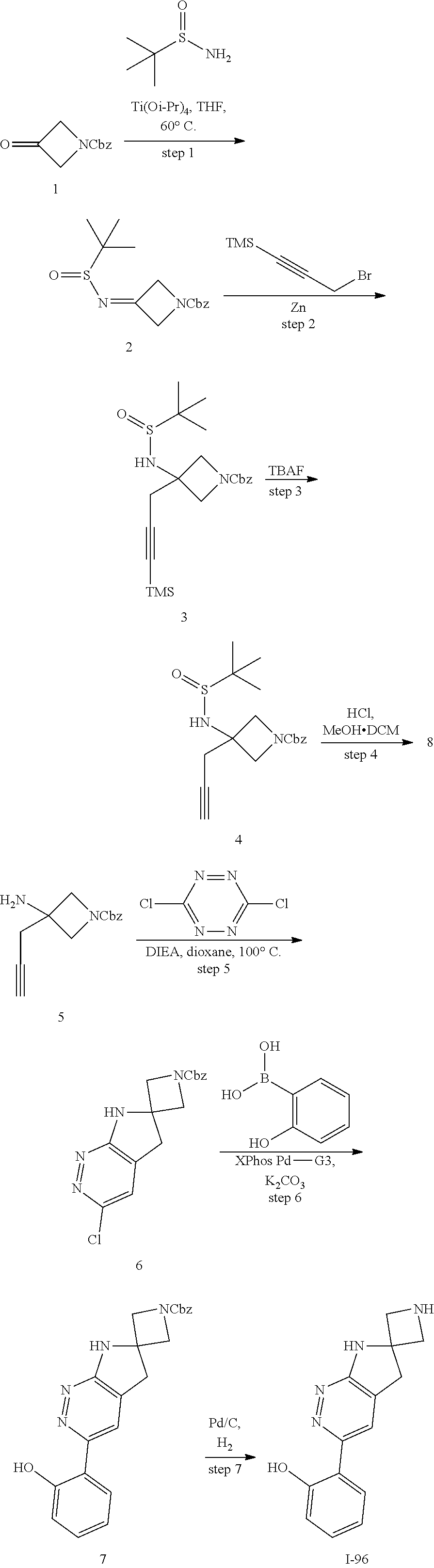

### Step 1: Preparation of benzyl 3-[(2-methylpropane-2-sulfinyl)imino]azetidine-1-carboxylate (intermediate 2)

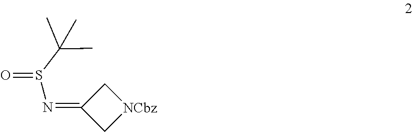 [SMILES: CC(C)(C)S(=O)N=C1CN(C(=O)OCc2ccccc2)C1]

To a stirred solution of benzyl 3-oxoazetidine-1-carboxylate (4 g, 19.492 mmol, 1.00 equiv) and tert-butanesulfinamide (2.36 g, 19.492 mmol, 1 equiv) in THF (40 mL) was added Ti(Oi-Pr)₄ (5.54 g, 19.492 mmol, 1 equiv). The resulting mixture was stirred for 1 h at 60 degrees C. The resulting mixture was diluted with EtOAc (300 mL), washed with water (100 mL×3), and the organic layer was dried over anhydrous Na₂SO₄. After filtration, the filtrate was concentrated under reduced pressure. The residue was purified by silica gel column chromatography, eluted with PE/EA (1:1) to afford intermediate 2 (3.58 g, 59.55%) as a white solid. LCMS (ESI) m/z: [M+H]⁺=309.

### Step 2: Preparation of benzyl 3-[(2-methylpropane-2-sulfinyl)amino]-3-[3-(trimethylsilyl)prop-2-yn-1-yl]azetidine-1-carboxylate (intermediate 3)

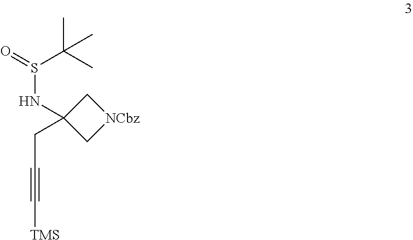 [SMILES: CC(C)(C)S(=O)NC1(CC#C[Si](C)(C)C)CN(C(=O)OCc2ccccc2)C1]

A mixture of intermediate 2 (1.5 g, 4.864 mmol, 1.00 equiv), (3-bromoprop-1-yn-1-yl)trimethylsilane (2.79 g, 14.592 mmol, 3 equiv) and Zn (0.95 g, 14.592 mmol, 3 equiv) in THF (20 mL) was stirred at 25 degrees C. for 16 hours. The mixture was diluted with EtOAc (80 mL) and washed with water (80 mL×3). The organic layer was dried over anhydrous sodium sulfate, filtered and concentrated to give a crude product. The crude product was purified by flash C18 chromatography, elution gradient 0 to 65% ACN in H₂O to give intermediate 3 (1.9 g, 92.91%) as a yellow solid. LCMS (ESI) m/z: [M+H]⁺=421.

### Step 3: Preparation of benzyl 3-[(2-methylpropane-2-sulfinyl)amino]-3-(prop-2-yn-1-yl)azetidine-1-carboxylate (intermediate 4)

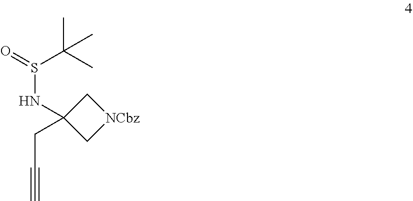 [SMILES: C#CCC1(NS(=O)C(C)(C)C)CN(C(=O)OCc2ccccc2)C1]

To a stirred solution of intermediate 3 (1.5 g, 3.566 mmol, 1.00 equiv) in DCM (20 mL) was added TBAF (17.83 mL, 17.830 mmol, 5 equiv). The resulting mixture was stirred for 1 h at room temperature. The resulting mixture was diluted with DCM (100 mL). The resulting mixture was washed with 5×50 mL of 5% HCl (aq). The organic layer was concentrated under vacuum. The crude product was used in the next step directly without further purification. LCMS (ESI) m/z: [M+H]⁺=349.

### Step 4: Preparation of benzyl 3-amino-3-(prop-2-yn-1-yl)azetidine-1-carboxylate (intermediate 5)

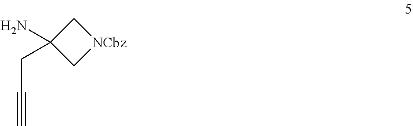 [SMILES: C#CCC1(N)CN(C(=O)OCc2ccccc2)C1]

To a stirred solution of intermediate 4 (600 mg, 1.722 mmol, 1.00 equiv) in DCM (10 mL) was added 4M HCl in MeOH (5 mL). The resulting mixture was stirred for 30 min at room temperature. The resulting mixture was concentrated under reduced pressure. The residue was purified by reverse phase flash chromatography with the following conditions: column, C18 silica gel; mobile phase, MeOH in water, 10% to 50% gradient in 30 min; detector, UV 200 nm to afford intermediate 5 (310 mg, 73.70%). LCMS (ESI) m/z: [M+H]⁺=245.

### Step 5: Preparation of benzyl 3′-chloro-5′,7′-dihydrospiro[azetidine-3,6′-pyrrolo[2,3-c]pyridazine]-1-carboxylate (intermediate 6)

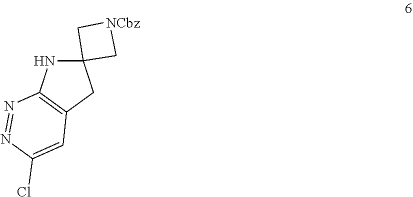 [SMILES: O=C(OCc1ccccc1)N1CC2(Cc3cc(Cl)nnc3N2)C1]

To a stirred solution of intermediate 5 (300 mg, 1.228 mmol, 1.00 equiv) and DIEA (634.86 mg, 4.912 mmol, 4 equiv) in dioxane (5 mL) was added dichloro-1,2,4,5-tetrazine (370.74 mg, 2.456 mmol, 2 equiv). The resulting mixture was stirred for 2 h at 100 degrees C. The resulting mixture was concentrated under vacuum. The residue was purified by silica gel column chromatography, eluted with CH₂Cl₂/MeOH (10:1) to afford intermediate 6 (220 mg, 54.16%) as a brown solid. LCMS (ESI) m/z: [M+H]⁺=331.

### Step 6: Preparation of benzyl 3′-(2-hydroxyphenyl)-5′,7′-dihydrospiro[azetidine-3,6′-pyrrolo[2,3-c]pyridazine]-1-carboxylate (intermediate 7)

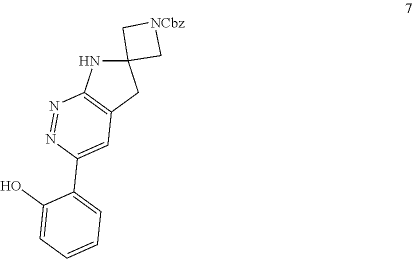 [SMILES: O=C(OCc1ccccc1)N1CC2(Cc3cc(-c4ccccc4O)nnc3N2)C1]

To a stirred solution of intermediate 6 (200 mg, 0.605 mmol, 1.00 equiv) and 2-hydroxyphenylboronic acid (250.20 mg, 1.815 mmol, 3 equiv) in dioxane (5 mL) and H₂O (1 mL) was added XPhos Pd G3 (102.36 mg, 0.121 mmol, 0.2 equiv) and K₂CO₃ (250.70 mg, 1.815 mmol, 3 equiv). The resulting mixture was stirred for 2 h at 90 degrees C. under an atmosphere of dry nitrogen. The resulting mixture was diluted with EtOAc (300 mL), washed with water (100 mL×3), and the organic layer was dried over anhydrous Na₂SO₄. After filtration, the filtrate was concentrated under reduced pressure. The residue was purified by silica gel column chromatography, eluted with CH₂Cl₂/MeOH (10:1) to afford intermediate 7 (121 mg, 51.52%) as a brown solid. LCMS (ESI) m/z: [M+H]⁺=389.

### Step 7: Preparation of 2-{5′,7′-dihydrospiro[azetidine-3,6′-pyrrolo[2,3-c]pyridazin]-3′-yl}phenol) (I-96)

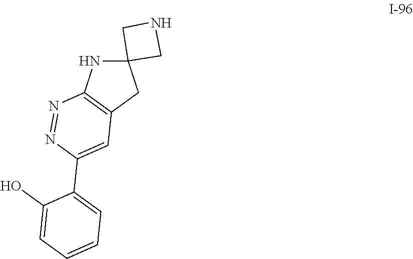 [SMILES: Oc1ccccc1-c1cc2c(nn1)NC1(CNC1)C2]

A stirred solution of intermediate 7 (20 mg, 0.051 mmol, 1.00 equiv) and Pd/C (10.96 mg, 0.102 mmol, 2 equiv) in MeOH (3 mL) was stirred for 2 h at room temperature under hydrogen atmosphere. The resulting mixture was filtered, and the filter cake was washed with MeOH (3×10 mL). The filtrate was concentrated under reduced pressure. The crude product (25 mg) was purified by Prep-HPLC with the following conditions (Column: XBridge Prep C18 OBD Column, 19*150 mm, 5 μm; Mobile Phase A: water (0.05% FA), Mobile Phase B: CH₃CN; Flow rate: 25 mL/min; Gradient: 4% B to 26% B in 8 min, 26% B; Wave Length: 254/220 nm; to afford I-96 (5 mg, 38%) as a brown solid. ¹H NMR (400 MHz, DMSO-d6) δ 14.19 (s, 1H), 8.30 (s, 1H), 8.06 (s, 1H), 7.84 (d, J=7.8 Hz, 1H), 7.25 (t, J=7.7 Hz, 1H), 6.90 (d, J=7.9 Hz, 2H), 4.02 (s, 1H), 3.76 (d, J=7.4 Hz, 2H), 3.46 (d, J=23.1 Hz, 4H). LCMS (ESI) m/z: [M+H]⁺=255.25.

### Preparation of (1S,3R)-3′-(2-hydroxyphenyl)-5′,7′-dihydrospiro[cyclobutane-1,6′-pyrrolo[2,3-c]pyridazine]-3-carboxylic acid (I-97) and (1R,3S)-3′-(2-hydroxyphenyl)-5′,7′-dihydrospiro[cyclobutane-1,6′-pyrrolo[2,3-c]pyridazine]-3-carboxylic acid (I-98)

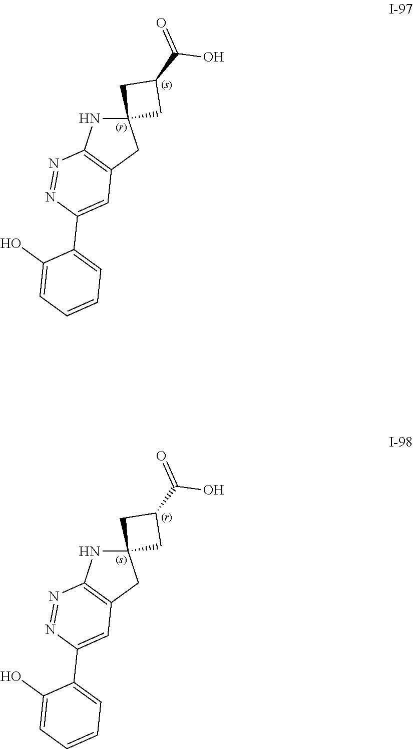 [SMILES: O=C(O)[C@H]1C[C@@]2(Cc3cc(-c4ccccc4O)nnc3N2)C1 ; O=C(O)[C@H]1C[C@]2(Cc3cc(-c4ccccc4O)nnc3N2)C1]

I-97 and I-98 were obtained as a yellow solid following the similar procedure as 1-96 starting from ethyl 3-oxocyclobutane-1-carboxylate and tert-butanesulfinamide. LCMS (ESI) m/z: [M+H]⁺=298.

### Preparation of 1-(3-(3-(2-hydroxyphenyl)-7H-pyrrolo[2,3-c]pyridazin-6-yl)azetidin-1-yl) prop-2-en-1-one (I-99)

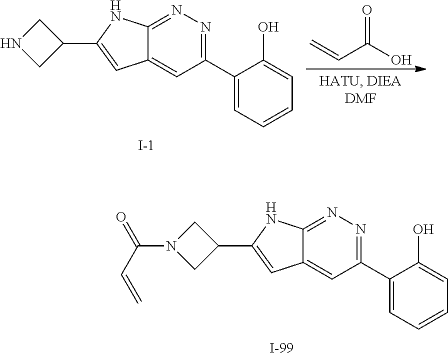

A mixture of acrylic acid (8.1 mg, 0.113 mmol, 1.00 equiv) in DMF (1 mL) was added HATU (64.2 mg, 0.170 mmol, 1.50 equiv) was stirred for 20 mins. 2-[6-(azetidin-3-yl)-7H-pyrrolo[2,3-c]pyridazin-3-yl]phenol (30.0 mg, 0.113 mmol, 1.00 equiv) and DIEA (43.6 mg, 0.339 mmol, 3.00 equiv) was added. The mixture was stirred for 2 hrs at room temperature. The reaction mixture without work-up was purified by Prep-HPLC with the following conditions: Column: XBridge Prep C₁₈ OBD Column, 19*150 mm, 5 μm; Mobile Phase A: water (10 MMOL/L NH₄HCO₃), Mobile Phase B: ACN; Flow rate: 25 mL/min; Gradient: 28% B to 37% B in 10 min, 37% B; Wave Length: 254/220 nm; afford I-99 (3.4 mg, 8.6%) as a yellow solid. LCMS (ESI) m/z: [M+H]⁺=321.35.

### Preparation of 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (I-6)

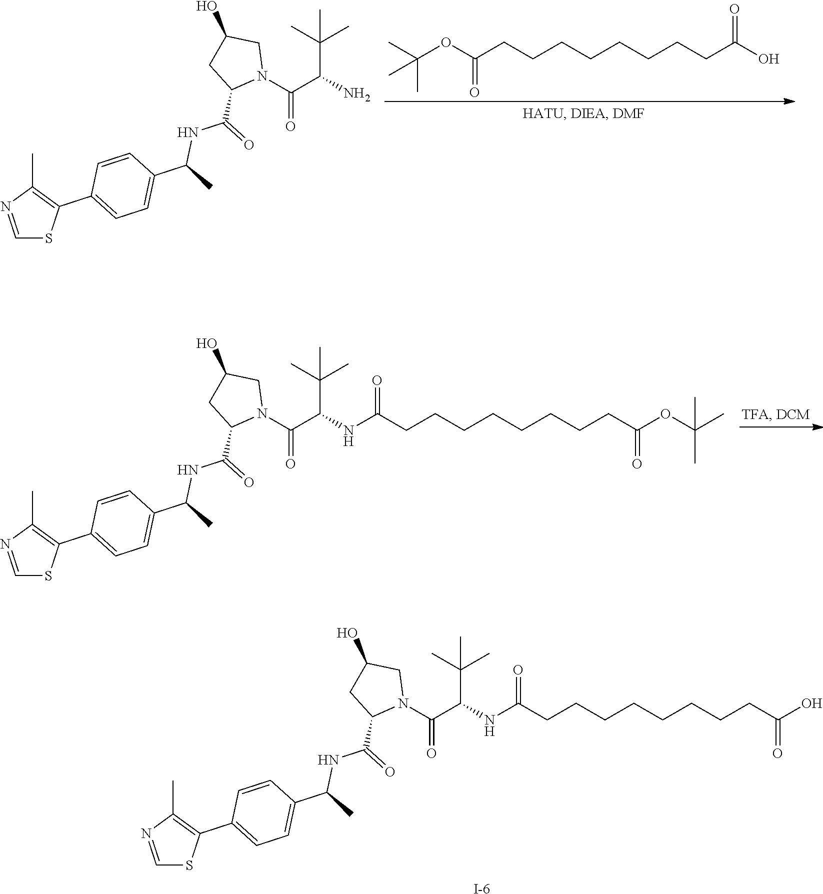

### Step 1: Preparation of tert-butyl 10-(((S)-1-((2S,4R)-4-hydroxy-2-(((S)—N-(4-(4-methylthiazol-5-yl)phenyl)ethyl)carbamoyl)pyrrolidin-1-yl)-3,3-dimethyl-1-oxobutan-2-yl)amino)-O-oxodecanoate

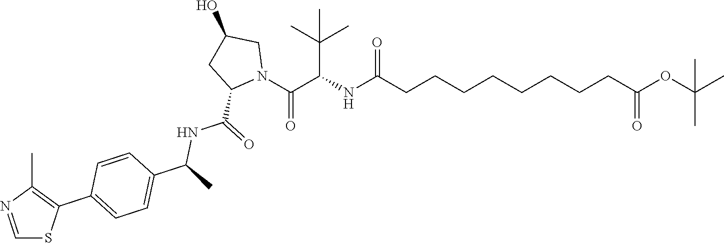 [SMILES: Cc1ncsc1-c1ccc([C@H](C)NC(=O)[C@@H]2C[C@@H](O)CN2C(=O)[C@@H](NC(=O)CCCCCCCCC(=O)OC(C)(C)C)C(C)(C)C)cc1]

To a stirred mixture of (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride (400 mg, 0.832 mmol) in DMF (5.00 mL) at room temperature was added 10-(tert-butoxy)-10-oxodecanoic acid (215 mg, 0.832 mmol), DIEA (322 mg, 2.50 mmol) and HATU (474 mg, 1.25 mmol). The resulting mixture was stirred for 2 h at room temperature. The residue was purified by reverse phase C18 flash chromatography (Water:ACN:FA) to afford tert-butyl 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoate as an off-white solid (440 mg, 65%). LCMS (ESI) m/z [M+H]⁺=685.

### Step 2: Preparation of 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (I-6)

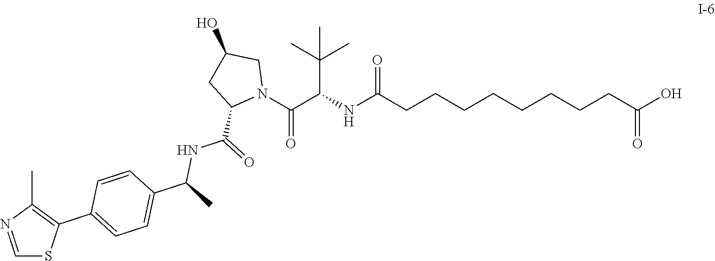 [SMILES: Cc1ncsc1-c1ccc([C@H](C)NC(=O)[C@@H]2C[C@@H](O)CN2C(=O)[C@@H](NC(=O)CCCCCCCCC(=O)O)C(C)(C)C)cc1]

To a stirred mixture of tert-butyl 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoate (430 mg, 0.628 mmol) in DCM (5.00 mL) was added TFA (1.50 mL) at room temperature. The resulting mixture was stirred for 1 h at room temperature. The residue was purified by reverse phase C18 flash chromatography (Water:ACN:FA) to afford 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (I-6, 280 mg, 70.8%). LCMS (ESI) m/z [M+H]⁺=629.

The following intermediates in Table 9 were prepared in a similar manner as described in the preparation of intermediate I-6 with (2S,4R)-1-[(2S)-2-amino-3,3-dimethylbutanoyl]-4-hydroxy-N-[(1S)-1-[4-(4-methyl-1,3-thiazol-5-yl)phenyl]ethyl]pyrrolidine-2-carboxamide hydrochloride and the appropriate carboxylic acid.

TABLE 9

| | | | LCMS |
|---|---|---|---|
| | Intermediate | | (ESI) |
| Structure | No. | Name | m/z |
| 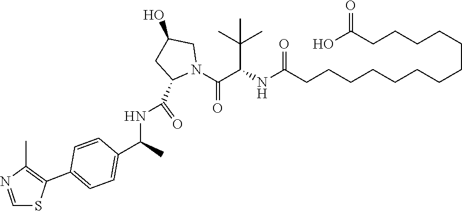 [SMILES: Cc1ncsc1-c1ccc([C@H](C)NC(=O)[C@@H]2C[C@@H](O)CN2C(=O)[C@@H](NC(=O)CCCCCCCCCCCCCC(=O)O)C(C)(C)C)cc1] | I-7 | 15-(((S)-1-((2S,4R)-4-hydroxy- 2-(((S)-1-(4-(4-methylthiazol-5- yl)phenyl)ethyl)carbamoyl) pyrrolidin-1-yl)-3,3-dimethyl-1- oxobutan-2-yl)amino)-15- oxopentadecanoic acid | 699 |
| 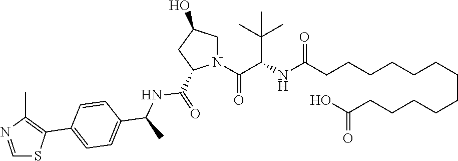 [SMILES: Cc1ncsc1-c1ccc([C@H](C)NC(=O)[C@@H]2C[C@@H](O)CN2C(=O)[C@@H](NC(=O)CCCCCCCCCCCCC(=O)O)C(C)(C)C)cc1] | I-8 | 14-(((S)-1-((2S,4R)-4-hydroxy- 2-(((S)-1-(4-(4-methylthiazol-5- yl)phenyl)ethyl)carbamoyl) pyrrolidin-1-yl)-3,3-dimethyl-1- oxobutan-2-yl)amino)-14- oxotetradecanoic acid | 685 |
| 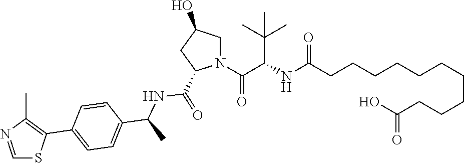 [SMILES: Cc1ncsc1-c1ccc([C@H](C)NC(=O)[C@@H]2C[C@@H](O)CN2C(=O)[C@@H](NC(=O)CCCCCCCCCCC(=O)O)C(C)(C)C)cc1] | I-9 | 12-(((S)-1-((2S,4R)-4-hydroxy- 2-(((S)-1-(4-(4-methylthiazol-5- yl)phenyl)ethyl)carbamoyl) pyrrolidin-1-yl)-3,3-dimethyl-1- oxobutan-2-yl)amino)-12- oxododecanoic acid | 657 |

### Example 2. Preparation of (2S,4R)-4-hydroxy-1-((S)-2-(10-(3-(3-(2-hydroxyphenyl)-7H-pyrrolo[2,3-c]pyridazin-6-yl)azetidin-1-yl)-10-oxodecanamido)-3,3-dimethylbutanoyl)-N—((S)-1-(4-(4-methylthiazol-5-yl)phenyl)ethyl)pyrrolidine-2-carboxamide (compound 1)

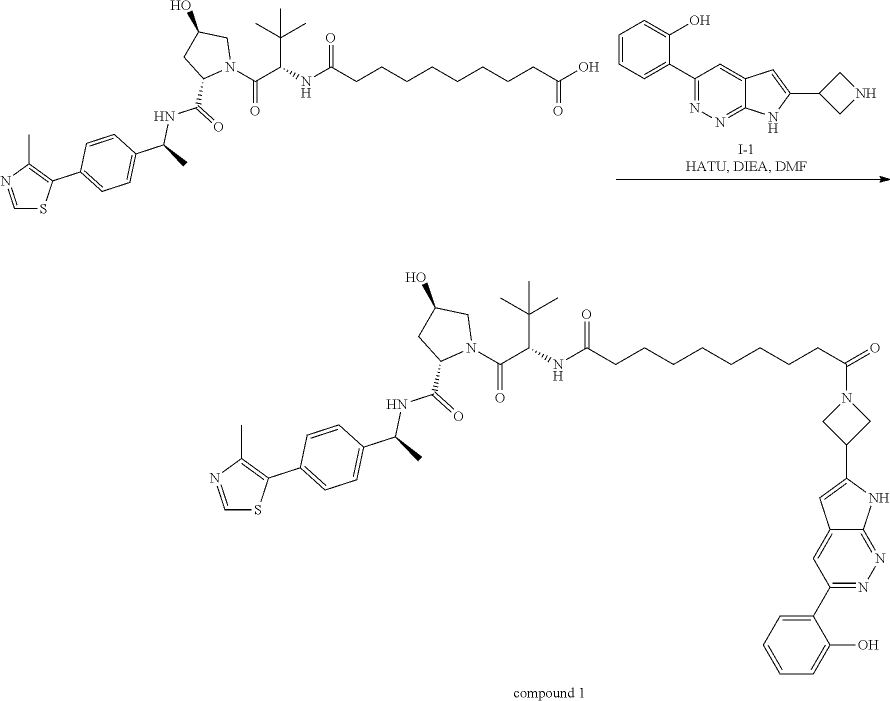

To a stirred mixture of 2-(6-(azetidin-3-yl)-7H-pyrrolo[2,3-c]pyridazin-3-yl)phenol (I-1, 8.47 mg, 0.032 mmol) and DIEA (12.3 mg, 0.095 mmol) in DMF (2 mL) at room temperature was added 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (20.0 mg, 0.032 mmol) and HATU (18.1 mg, 0.048 mmol). The resulting mixture was stirred at room temperature overnight. The crude product was purified by Prep-HPLC (Water:ACN:FA) to afford compound 1 (7 mg, 98.2%) as an off-white solid. ¹H NMR (300 MHz, DMSO-d₆) δ 13.01 (br s, 1H), 8.99 (s, 1H), 8.61 (s, 1H), 8.38 (d, J=7.8 Hz, 1H), 7.91 (s, 1H), 7.79 (d, J=9.3 Hz, 1H), 7.47-7.32 (m, 5H), 7.06-6.96 (m, 2H), 6.79 (s, 1H), 4.99-4.84 (m, 1H), 4.63-4.48 (m, 2H), 4.47-4.37 (m, 2H), 4.37-4.16 (m, 3H), 4.12-4.03 (m, 1H), 3.60 (s, 2H), 2.46 (s, 3H), 2.33-2.19 (m, 1H), 2.18-1.96 (m, 4H), 1.88-1.72 (m, 1H), 1.58-1.42 (m, 4H), 1.38 (d, J=7.0 Hz, 3H), 1.26 (s, 8H), 0.94 (s, 9H). LCMS (ESI) m/z [M+H]⁺=877.20.

The compounds in Table 10 were prepared using procedures similar to those used for the preparation of compound 1 using the appropriate and amine and carboxylic acid OR

TABLE 10

| | | LCMS | |
|---|---|---|---|
| | | (ESI) | |
| No. | Name | m/z | ¹H NMR |
| 2 | (2S,4R)-4-hydroxy-1-((S)-2-(10-(3- | 863.2 | ¹H NMR (400 MHz, DMSO-d₆) δ 12.93 (d, J = |
| | (3-(2-hydroxyphenyl)-7H- | | 18.6 Hz, 1H), 8.98 (s, 1H), 8.58 (d, J = 14.1 |
| | pyrrolo[2,3-c]pyridazin-6-yl)azetidin- | | Hz, 2H), 7.96 (s, 1H), 7.84 (d, J = 9.4 Hz, 1H), |
| | 1-yl)-10-oxodecanamido)-3,3- | | 7.49-7.26 (m, 5H), 7.03-6.93 (m, 2H), 6.73 |
| | dimethylbutanoyl)-N-(4-(4- | | (s, 1H), 5.12 (d, J = 7.8 Hz, 1H), 4.55 (t, J = |
| | methylthiazol-5- | | 8.7 Hz, 2H), 4.43 (q, J = 7.3 Hz, 2H), 4.37- |
| | yl)benzyl)pyrrolidine-2-carboxamide | | 4.11 (m, 5H), 4.06 (dd, J = 9.0, 6.0 Hz, 1H), |
| | | | 3.68-3.63 (m, 2H), 2.44 (s, 3H), 2.26 (dt, J = |
| | | | 14.6, 7.5 Hz, 1H), 2.10 (q, J = 7.5 Hz, 3H), |
| | | | 2.01 (d, J = 8.8 Hz, 1H), 1.90 (ddd, J = 12.8, |
| | | | 8.5, 4.5 Hz, 1H), 1.49 (s, 4H), 1.25 (s, 8H), |
| | | | 0.93 (s, 9H). |
| 3 | (2S,4R)-4-hydroxy-1-((S)-2-(8-(3-(3- | 835.4 | ¹H NMR (400 MHz, DMSO-d₆) δ 14.1 ( s,1H), |
| | (2-hydroxyphenyl)-7H-pyrrolo[2,3- | | 12.82 (s, 1H), 8.98 (s, 1H), 8.58 (d, J = 10.3 |
| | c]pyridazin-6-yl)azetidin-1-yl)-8- | | Hz, 2H), 8.00 (s, 1H), 7.86 (d, J = 9.3 Hz, 1H), |
| | oxooctanamido)-3,3- | | 7.40 (q, J = 8.3 Hz, 4H), 7.31 (t, J = 7.9 Hz, |
| | dimethylbutanoyl)-N-(4-(4- | | 1H), 6.97 (dd, J = 11.4, 7.8 Hz, 2H), 6.69 (s, |
| | methylthiazol-5- | | 1H), 5.13 (s, 1H), 4.55 (dt, J = 8.7, 3.9 Hz, |
| | yl)benzyl)pyrrolidine-2-carboxamide | | 2H), 4.48-4.38 (m, 2H), 4.38-4.13 (m, 5H), |
| | | | 4.06 (dd, J = 9.0, 6.0 Hz, 1H), 3.66 (d, J = 4.2 |
| | | | Hz, 2H), 2.44 (s, 3H), 2.26 (dt, J = 14.7, 7.6 |
| | | | Hz, 1H), 2.18-1.98 (m, 4H), 1.90 (ddd, J = |
| | | | 12.9, 8.5, 4.5 Hz, 1H), 1.53-1.44 (m, 4H), |
| | | | 1.25 (d, J = 12.2 Hz, 4H), 0.93 (s, 9H). |
| 4 | (2S,4R)-4-hydroxy-1-((S)-2-(12-(3- | 891.5 | ¹H NMR (400 MHz, DMSO-d₆) δ 14.06 (s, 1H), |
| | (3-(2-hydroxyphenyl)-7H- | | 12.74 (s, 1H), 8.98 (s, 1H), 8.58 (d, J = 8.9 Hz, |
| | pyrrolo[2,3-c]pyridazin-6-yl)azetidin- | | 2H), 8.04 (dd, J = 8.0, 1.7 Hz, 1H), 7.85 (d, J = |
| | 1-yl)-12-oxododecanamido)-3,3- | | 9.3 Hz, 1H), 7.40 (q, J = 8.3 Hz, 4H), 7.29 |
| | dimethylbutanoyl)-N-(4-(4- | | (ddd, J = 8.5, 7.1, 1.6 Hz, 1H), 7.00-6.91 (m, |
| | methylthiazol-5- | | 2H), 6.66 (s, 1H), 5.12 (d, J = 3.7 Hz, 1H), |
| | yl)benzyl)pyrrolidine-2-carboxamide | | 4.54 (dt, J = 8.4, 3.8 Hz, 2H), 4.48-4.38 (m, |
| | | | 2H), 4.37-4.09 (m, 5H), 4.06 (dd, J = 8.9, 6.1 |
| | | | Hz, 1H), 3.66 (d, J = 4.8 Hz, 1H), 3.51 (s, 1H), |
| | | | 2.44 (s, 3H), 2.25 (dt, J = 14.7, 7.5 Hz, 1H), |
| | | | 2.18-1.95 (m, 4H), 1.90 (ddd, J = 12.7, 8.5, |
| | | | 4.6 Hz, 1H), 1.58-1.37 (m, 4H), 1.24 (s, |
| | | | 12H), 0.93 (s, 9H). |
| 5 | (2S,4R)-4-hydroxy-1-((S)-2-(11-(3- | 877.4 | ¹H NMR (400 MHz, DMSO-d₆) δ 14.06 (s, 1H), |
| | (3-(2-hydroxyphenyl)-7H- | | 12.77-12.71 (m, 1H), 8.98 (s, 1H), 8.58 (d, J = |
| | pyrrolo[2,3-c]pyridazin-6-yl)azetidin- | | 7.9 Hz, 2H), 8.04 (dd, J = 8.1, 1.7 Hz, 1H), |
| | 1-yl)-11-oxoundecanamido)-3,3- | | 7.85 (d, J = 9.4 Hz, 1H), 7.40 (q, J = 8.2 Hz, |
| | dimethylbutanoyl)-N-(4-(4- | | 4H), 7.34-7.25 (m, 1H), 7.00-6.91 (m, 2H), |
| | methylthiazol-5- | | 6.65 (s, 1H), 5.13 (d, J = 3.5 Hz, 1H), 4.54 (dt, |
| | yl)benzyl)pyrrolidine-2-carboxamide | | J = 8.5, 3.8 Hz, 2H), 4.43 (q, J = 7.4 Hz, 2H), |
| | | | 4.37-4.09 (m, 4H), 4.06 (dd, J = 9.0, 6.1 Hz, |
| | | | 1H), 3.76-3.46 (m, 3H), 2.44 (s, 3H), 2.36- |
| | | | 2.20 (m, 1H), 2.20-1.98 (m, 4H), 1.97-1.90 (m, |
| | | | 1H), 1.56-1.41 (m, 4H), 1.24 (d, J = 5.1 Hz, |
| | | | 10H), 0.93 (s, 9H). |
| 6 | (2S,4R)-4-hydroxy-1-((S)-2-(5-(3-(3- | 793.3 | ¹H NMR (400 MHz, DMSO-d₆) δ 14.05 (s, 1H), |
| | (2-hydroxyphenyl)-7H-pyrrolo[2,3- | | 12.73 (s, 1H), 8.98 (s, 1H), 8.61-8.51 (m, |
| | c]pyridazin-6-yl)azetidin-1-yl)-5- | | 2H), 8.03 (d, J = 8.0 Hz, 1H), 7.91 (d, J = 9.3 |
| | oxopentanamido)-3,3- | | Hz, 1H), 7.46-7.34 (m, 4H), 7.32-7.26 (m, |
| | dimethylbutanoyl)-N-(4-(4- | | 1H), 7.01-6.91 (m, 2H), 6.68-6.64 (m, 1H), |
| | methylthiazol-5- | | 5.14 (d, J = 3.6 Hz, 1H), 4.59-4.48 (m, 2H), |
| | yl)benzyl)pyrrolidine-2-carboxamide | | 4.48-4.38 (m, 2H), 4.38-4.21 (m, 3H), 4.21- |
| | | | 4.02 (m, 3H), 3.67 (s, 2H), 2.44 (s, 3H), 2.31- |
| | | | 2.16 (m, 2H), 2.14-1.98 (m, 3H), 1.96- |
| | | | 1.85 (m, 1H), 1.80-1.67 (m, 2H), 0.94 (d, J = |
| | | | 3.7 Hz, 9H). |
| 7 | 2-(2,6-dioxopiperidin-3-yl)-4-((5-(3- | 623.1 | ¹H NMR (400 MHz, DMSO-d₆) δ 14.06 (s, 1H), |
| | (3-(2-hydroxyphenyl)-7H- | | 12.75 (s, 1H), 11.11 (s, 1H), 8.59 (s, 1H), 8.04 |
| | pyrrolo[2,3-c]pyridazin-6-yl)azetidin- | | (dd, J = 8.0, 1.6 Hz, 1H), 7.82 (dd, J = 8.5, 7.3 |
| | 1-yl)-5-oxopentyl)oxy)isoindoline- | | Hz, 1H), 7.53 (d, J = 8.5 Hz, 1H), 7.45 (d, J = |
| | 1,3-dione | | 7.2 Hz, 1H), 7.29 (ddd, J = 8.5, 7.1, 1.6 Hz, |
| | | | 1H), 7.01-6.91 (m, 2H), 6.65 (s, 1H), 5.08 |
| | | | (dd, J = 12.9, 5.4 Hz, 1H), 4.61-4.51 (m, 1H), |
| | | | 4.39-4.12 (m, 5H), 4.07 (dd, J = 8.9, 6.1 Hz, |
| | | | 1H), 2.93-2.80 (m, 1H), 2.61-2.52 (m, 2H), |
| | | | 2.23 (t, J = 7.4 Hz, 2H), 2.06-1.97 (m, 1H), |
| | | | 1.85-1.65 (m, 4H). |
| 8 | 2-(2,6-dioxopiperidin-3-yl)-4-(4-(3- | 609.1 | ¹H NMR (400 MHz, DMSO-d₆) δ 14.05 (s, 1H), |
| | (3-(2-hydroxyphenyl)-7H- | | 12.73 (s, 1H), 11.11 (s, 1H), 8.56 (s, 1H), 8.04 |
| | pyrrolo[2,3-c]pyridazin-6-yl)azetidin- | | (dd, J = 8.0, 1.6 Hz, 1H), 7.81 (dd, J = 8.5, 7.3 |
| | 1-yl)-4-oxobutoxy)isoindoline-1,3- | | Hz, 1H), 7.55 (d, J = 8.5 Hz, 1H), 7.43 (d, J = |
| | dione | | 7.2 Hz, 1H), 7.30 (ddd, J = 8.5, 7.1, 1.6 Hz, |
| | | | 1H), 6.96 (t, J = 7.9 Hz, 2H), 6.62 (s, 1H), 5.08 |
| | | | (dd, J = 12.9, 5.4 Hz, 1H), 4.57 (t, J = 8.5 Hz, |
| | | | 1H), 4.39-4.22 (m, 4H), 4.21-4.04 (m, 2H), |
| | | | 2.94-2.81 (m, 1H), 2.62-2.53 (m, 2H), 2.35 |
| | | | (t, J = 7.0 Hz, 2H), 2.06-1.97 (m, 3H). |
| 9 | 2-(2,6-dioxopiperidin-3-yl)-5-((9-(3- | 678.1 | ¹H NMR (400 MHz, DMSO-d₆) δ 14.06 (s, 1H), |
| | (3-(2-hydroxyphenyl)-7H- | | 12.74 (s, 1H), 11.06 (s, 1H), 8.59 (s, 1H), 8.04 |
| | pyrrolo[2,3-c]pyridazin-6-yl)azetidin- | | (dd, J = 8.0, 1.6 Hz, 1H), 7.55 (d, J = 8.4 Hz, |
| | 1-yl)-9-oxononyl)amino) isoindoline- | | 1H), 7.34-7.25 (m, 1H), 7.10 (t, J = 5.4 Hz, |
| | 1,3-dione | | 1H), 7.00-6.91 (m, 3H), 6.84 (dd, J = 8.5, 2.1 |
| | | | Hz, 1H), 6.66 (d, J = 1.8 Hz, 1H), 5.03 (dd, J = |
| | | | 12.9, 5.4 Hz, 1H), 4.54 (t, J = 8.5 Hz, 1H), 4.38- |
| | | | 4.22 (m, 2H), 4.21-4.10 (m, 1H), 4.10- |
| | | | 4.02 (m, 1H), 3.14 (q, J = 6.6 Hz, 2H), 2.94- |
| | | | 2.80 (m, 1H), 2.61-2.51 (m, 2H), 2.09 (t, J = |
| | | | 7.4 Hz, 2H), 2.04-1.91 (m, 1H), 1.61-1.43 |
| | | | (m, 4H), 1.40-1.21 (m, 8H). |
| 10 | (2S,4R)-4-hydroxy-1-((S)-2-(9-(3-(3- | 849.2 | ¹H NMR (400 MHz, DMSO-d₆) δ 14.05 (s, 1H), |
| | (2-hydroxyphenyl)-7H-pyrrolo[2,3- | | 12.73 (s, 1H), 8.98 (s, 1H), 8.63-8.50 (m, |
| | c]pyridazin-6-yl)azetidin-1-yl)-9- | | 2H), 8.04 (d, 1H), 7.84 (d, J = 9.4 Hz, 1H), |
| | oxononanamido)-3,3- | | 7.40 (q, J = 8.2 Hz, 4H), 7.32-7.26 (m, 1H), |
| | dimethylbutanoyl)-N-(4-(4- | | 6.96 (dd, J = 9.9, 7.9 Hz, 2H), 6.65 (s, 1H), |
| | methylthiazol-5- | | 5.12 (d, J = 3.6 Hz, 1H), 4.58-4.51 (m, 2H), |
| | yl)benzyl)pyrrolidine-2-carboxamide | | 4.47-4.39 (m, 2H), 4.36-4.21 (m, 4H), 4.20- |
| | | | 4.14 (m, 1H), 4.06 (dd, J = 9.0, 6.1 Hz, 1H), |
| | | | 3.66 (d, J = 4.6 Hz, 2H), 2.44 (s, 3H), 2.29- |
| | | | 2.21 (m, 1H), 2.14-2.00 (m, 4H), 1.93-1.86 |
| | | | (m, 1H), 1.55-1.43 (m, 4H), 1.26 (s, 6H), |
| | | | 0.93 (s, 9H). |
| 11 | (2S,4R)-4-hydroxy-1-((S)-2-(15-(3- | 946.9 | ¹H NMR (300 MHz, DMSO-d₆) δ 12.87 (s, 1H), |
| | (3-(2-hydroxyphenyl)-7H- | | 8.99 (s, 1H), 8.60 (s, 1H), 8.38 (d, J = 8.0 Hz, |
| | pyrrolo[2,3-c]pyridazin-6-yl)azetidin- | | 1H), 7.97 (s, 1H), 7.79 (d, J = 9.1 Hz, 1H), |
| | 1-yl)-15-oxopentadecanamido)-3,3- | | 7.48-7.27 (m, 5H), 6.98 (t, J = 8.5 Hz, 2H), |
| | dimethylbutanoyl)-N-((S)-1-(4-(4- | | 6.73 (s, 1H), 4.92 (t, J = 7.1 Hz, 1H), 4.62- |
| | methylthiazol-5- | | 4.42 (m, 2H), 4.39-4.24 (m, 4H), 4.19 (d, J = |
| | yl)phenyl)ethyl)pyrrolidine-2- | | 7.4 Hz, 1H), 4.11-4.00 (m, 1H), 3.60 (s, 2H), |
| | carboxamide | | 2.46 (s, 3H), 2.32-2.18 (m, 1H), 2.15-2.02 |
| | | | (m, 3H), 1.99 (d, J = 4.9 Hz, 1H), 1.88-1.68 |
| | | | (m, 1H), 1.48 (s, 4H), 1.38 (d, J = 7.0 Hz, 3H), |
| | | | 1.24 (s, 20H), 0.94 (s, 9H). |
| 12 | (2S,4R)-4-hydroxy-1-((S)-2-(14-(3- | 932.9 | ¹H NMR (300 MHz, DMSO-d₆) d 14.06 (s, 1H), |
| | (3-(2-hydroxyphenyl)-7H- | | 12.74 (s, 1H), 8.99 (s, 1H), 8.60 (s, 1H), 8.38 |
| | pyrrolo[2,3-c]pyridazin-6-yl)azetidin- | | (d, J = 7.8 Hz, 1H), 8.04 (d, J = 8.2 Hz, 1H), |
| | 1-yl)-14-oxotetradecanamido)-3,3- | | 7.79 (d, J = 9.2 Hz, 1H), 7.47-7.36 (m, 4H), |
| | dimethylbutanoyl)-N-((S)-1-(4-(4- | | 7.34-7.24 (m, 1H), 6.96 (t, J = 8.1 Hz, 2H), |
| | methylthiazol-5- | | 6.67 (s, 1H), 5.10 (d, J = 3.6 Hz, 1H), 4.97- |
| | yl)phenyl)ethyl)pyrrolidine-2- | | 4.86 (m, 1H), 4.60-4.46 (m, 2H), 4.48-4.16 |
| | carboxamide | | (m, 4H), 4.22-4.01 (m, 2H), 3.60 (s, 2H), |
| | | | 2.46 (s, 3H), 2.31-2.15 (m, 1H), 2.09 (t, J = |
| | | | 7.2 Hz, 3H), 1.99 (d, J = 7.0 Hz, 1H), 1.79 (s, |
| | | | 1H), 1.48 (s, 4H), 1.38 (d, J = 6.9 Hz, 3H), |
| | | | 1.24 (s, 16H), 0.94 (s, 9H). |
| 13 | (2S,4R)-4-hydroxy-1-((S)-2-(12-(3- | 905.4 | ¹H NMR (400 MHz, DMSO-d₆) δ 14.08 (s, 1H), |
| | (3-(2-hydroxyphenyl)-7H- | | 12.71 (s, 1H), 8.98 (s, 1H), 8.58 (s, 1H), 8.37 |
| | pyrrolo[2,3-c]pyridazin-6-yl)azetidin- | | (d, J = 7.8 Hz, 1H), 8.04 (dd, J = 8.2, 1.6 Hz, |
| | 1-yl)-12-oxododecanamido)-3,3- | | 1H), 7.78 (d, J = 9.3 Hz, 1H), 7.48-7.32 (m, |
| | dimethylbutanoyl)-N-((S)-1-(4-(4- | | 4H), 7.35-7.24 (m, 1H), 7.01-6.91 (m, 2H), |
| | methylthiazol-5- | | 6.65 (s, 1H),5.10 (s, 1H), 4.92 (q, J = 7.3 Hz, |
| | yl)phenyl)ethyl)pyrrolidine-2- | | 1H), 4.58-4.46 (m, 2H), 4.42 (t, J = 8.0 Hz, |
| | carboxamide | | 1H), 4.38-4.32 (m, 1H), 4.32-4.23 (m, 2H), |
| | | | 4.15 (t, J = 7.5 Hz, 1H), 4.06 (dd, J = 9.1, 6.0 |
| | | | Hz, 1H), 3.60 (s, 2H),2.37-2.30 (m, 3H), 2.31- |
| | | | 2.19 (m, 1H), 2.15-2.05 (m, 3H), 2.05- |
| | | | 1.96 (m, 1H), 1.84-1.73 (m, 1H), 1.60-1.42 |
| | | | (m, 4H), 1.37 (d, J = 7.0 Hz, 3H), 1.24 (s, |
| | | | 12H), 0.93 (s, 9H). |

### Example 3. Preparation of (2S,4R)-4-hydroxy-1-((R)-2-(3-(2-(3-(3-(2-hydroxyphenyl)-7H-pyrrolo[2,3-c]pyridazin-6-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide formate (compound 14)

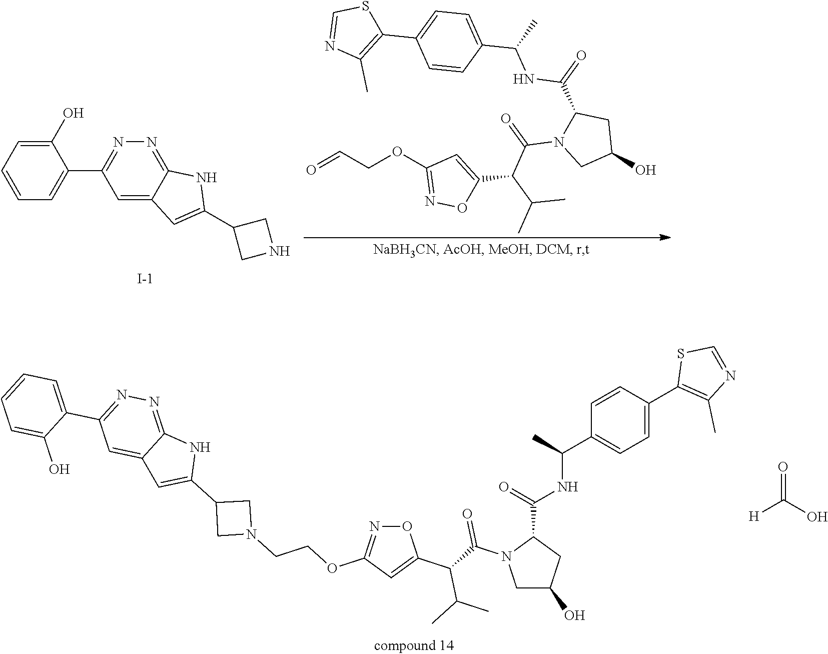 [SMILES: Oc1ccccc1-c1cc2cc(C3CNC3)[nH]c2nn1 ; Cc1ncsc1-c1ccc([C@H](C)NC(=O)[C@@H]2C[C@@H](O)CN2C(=O)[C@@H](c2cc(OCC=O)no2)C(C)C)cc1 ; [I-] ; Cc1ncsc1-c1ccc([C@H](C)NC(=O)[C@@H]2C[C@@H](O)CN2C(=O)[C@@H](c2cc(OCCN3CC(c4cc5cc(-c6ccccc6O)nnc5[nH]4)C3)no2)C(C)C)cc1 ; O=CO]

To a solution of 2-(6-(azetidin-3-yl)-7H-pyrrolo[2,3-c]pyridazin-3-yl)phenol (I-1, 6.60 mg, 0.025 mmol) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (13.4 mg, 0.025 mmol) in a mixture of MeOH (2.00 mL) and DCM (2.00 mL) was added AcOH (0.10 mL, 1.75 mmol) until pH=6. NaBH₃CN (6.23 mg, 0.100 mmol) was then added to the reaction mixture and the mixture was stirred at room temperature for 10 h. The mixture solution was purified by Prep-HPLC to afford compound 14 (10 mg, 47.0%) as a white solid. ¹H NMR (400 MHz, DMSO-d₆) δ 14.01 (s, 1H), 12.66 (s, 1H), 8.99 (s, 1H), 8.61 (s, 1H), 8.41 (d, J=7.9 Hz, 1H), 8.14 (d, J=1.2 Hz, 1H), 8.05 (d, J=7.8 Hz, 1H), 7.44 (d, J=7.9 Hz, 2H), 7.36 (d, J=8.1 Hz, 2H), 7.30 (t, J=7.5 Hz, 1H), 6.96 (t, J=8.3 Hz, 2H), 6.66 (s, 1H), 6.13 (s, 1H), 5.12 (d, J=3.5 Hz, 1H), 4.92 (q, J=7.1 Hz, 1H), 4.36 (t, J=7.9 Hz, 6H), 4.08 (s, 2H), 3.74-3.64 (m, 3H), 2.48 (s, 3H), 2.30-2.18 (m, 1H), 2.04 (dd, J=19.4, 8.4 Hz, 1H), 1.78 (dd, J=12.7, 8.2, Hz, 1H), 1.5 (s, 1H), 1.37 (d, J=7.0 Hz, 3H), 0.97 (t, J=6.0 Hz, 4H), 0.82 (dd, J=14.1, 6.7 Hz, 4H). LCMS (ESI) m/z: [M+H]⁺=791.3.

### Example 4. Preparation of (2S,4R)-4-hydroxy-1-((R)-2-(3-(2-(3-(3-(2-hydroxyphenyl)-7H-pyrrolo[2,3-c]pyridazin-5-yl)azetidin-1-yl)ethoxy)isoxazol-5-yl)-3-methylbutanoyl)-N—((S)-1-(4-(4-methylthiazol-5-yl)phenyl)ethyl)pyrrolidine-2-carboxamide formate (compound 15)

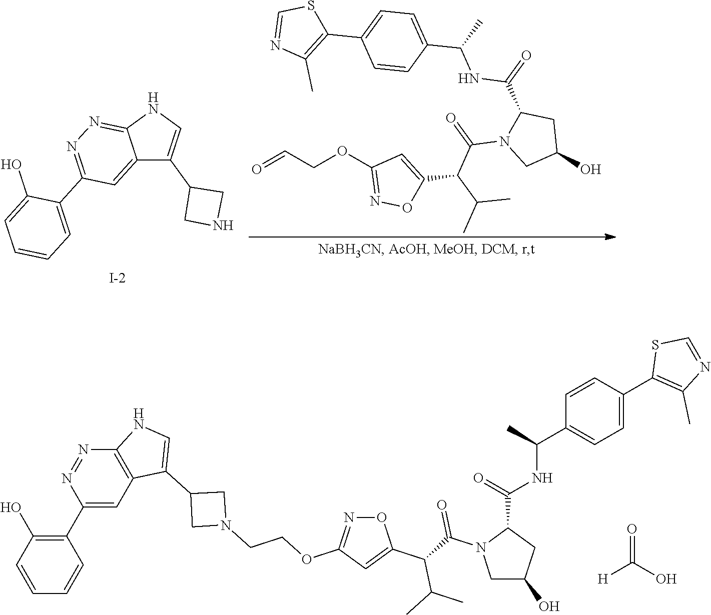

Compound 15 was prepared according to the synthetic procedure described for the preparation of compound 14 beginning from 2-(5-(azetidin-3-yl)-7H-pyrrolo[2,3-c]pyridazin-3-yl)phenol (I-2) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide. ¹H NMR (400 MHz, DMSO-d₆) δ 14.01 (s, 1H), 12.51 (s, 1H), 8.98 (s, 1H), 8.81 (s, 1H), 8.40 (d, J=7.7 Hz, 1H), 8.11-8.03 (m, 2H), 7.47-7.42 (m, 2H), 7.39-7.34 (m, 2H), 7.30 (td, J=7.6, 1.6 Hz, 1H), 7.02-6.93 (m, 2H), 6.10 (s, 1H), 5.10 (d, J=3.6 Hz, 1H), 4.90 (q, J=7.2 Hz, 1H), 4.36 (t, J=7.9 Hz, 1H), 4.31-4.22 (m, 3H), 3.99 (s, 3H), 3.71 (dd, 1H), 3.66 (d, J=9.7 Hz, 1H), 3.55 (s, 1H), 3.45 (d, J=10.9 Hz, 2H), 3.06 (s, 2H), 2.45 (d, J=2.6 Hz, 3H), 2.24 (d, J=9.7 Hz, 1H), 2.07-1.97 (m, 1H), 1.83-1.74 (m, 1H), 1.37 (d, J=7.0 Hz, 3H), 0.95 (d, J=6.4 Hz, 3H), 0.80 (dd, J=13.7, 6.7 Hz, 3H). LCMS (ESI) m/z: [M+H]⁺=791.35.

The compounds in Table 11 were prepared following protocols analogous to those above for compound 15 (Procedure B) using the appropriate and amine and aldehyde (or ketone) or according to the procedure analogous to those above for compound 1 (Procedure A) using the appropriate amine and carboxylic acid.

TABLE 11

| | | | LCMS | |
|---|---|---|---|---|
| | | | (ESI) | |
| No. | Proc. | Name | m/z | ¹H NMR |
| 16 | A | (2S,4R)-4-hydroxy-1-((S)-2- | 751.3 | 1H NMR (400 MHz, DMSO-d6) δ |
| | | (3-(3-(3-(2-hydroxyphenyl)- | | 14.13 (s, 1H), 12.57 (s, 1H), 8.96 (s, |
| | | 7H-pyrrolo[2,3-c]pyridazin-6- | | 1H), 8.59 (t, J = 6.1 Hz, 1H), 8.54 |
| | | yl)azetidin-1- | | (s, 1H), 8.31 (d, J = 9.3 Hz, 1H), |
| | | yl)propanamido)-3,3- | | 8.12-7.96 (m, 1H), 7.46-7.34 (m, |
| | | dimethylbutanoyl)-N-(4-(4- | | 4H), 7.36-7.24 (m, 1H), 6.95 (dd, |
| | | methylthiazol-5- | | J = 11.4, 7.9 Hz, 2H), 6.60 (s, 1H), |
| | | yl)benzyl)pyrrolidine-2- | | 5.15 (s, 1H), 4.55 (d, J = 9.3 Hz, |
| | | carboxamide | | 1H), 4.49-4.39 (m, 2H), 4.36 (s, |
| | | | | 1H), 4.23 (dd, J = 15.8, 5.4 Hz, 1H), |
| | | | | 3.86 (s, 1H), 3.68 (dd, J = 11.2, 7.3 |
| | | | | Hz, 3H), 3.34 (s, 3H), 2.50 (d, J = |
| | | | | 1.9 Hz, 2H), 2.42 (s, 3H), 2.28 (d, J = |
| | | | | 37.7 Hz, 2H), 2.04 (t, J = 10.3 Hz, |
| | | | | 1H), 1.99-1.91 (m, 1H), 0.95 (s, |
| | | | | 9H). |
| 17 | A | (2S,4R)-4-hydroxy-1-((S)-2- | 919.2 | 1H NMR (400 MHz, DMSO-d6) δ |
| | | (14-(3-(3-(2-hydroxyphenyl)- | | 14.06 (s, 1H), 12.73 (s, 1H), 8.98 (s, |
| | | 7H-pyrrolo[2,3-c]pyridazin-6- | | 1H), 8.58 (d, J = 9.0 Hz, 2H), 8.07- |
| | | yl)azetidin-1-yl)-14- | | 8.01 (m, 1H), 7.84 (d, J = 9.3 Hz, |
| | | oxotetradecanamido)-3,3- | | 1H), 7.40 (q, J = 8.2 Hz, 4H), 7.29 |
| | | dimethylbutanoyl)-N-(4-(4- | | (ddd, J = 8.5, 7.2, 1.6 Hz, 1H), 7.00- |
| | | methylthiazol-5- | | 6.91 (m, 2H), 6.66 (d, J = 1.8 Hz, |
| | | yl)benzyl)pyrrolidine-2- | | 1H), 5.13 (d, J = 3.5 Hz, 1H), 4.54 |
| | | carboxamide | | (dt, J = 8.6, 3.8 Hz, 2H), 4.43 (q, J = |
| | | | | 7.4, 6.3 Hz, 2H), 4.28 (ddd, J = |
| | | | | 28.8, 12.7, 5.9 Hz, 4H), 4.22-4.09 |
| | | | | (m, 2H), 4.06 (dd, J = 8.9, 6.1 Hz, |
| | | | | 1H), 3.66 (t, J = 3.0 Hz, 2H), 2.44 |
| | | | | (s, 3H), 2.25 (dt, J = 14.8, 7.5 Hz, |
| | | | | 1H), 2.10 (q, J = 8.2, 7.5 Hz, 3H), |
| | | | | 2.01 (d, J = 8.9 Hz, 1H), 1.95-1.84 |
| | | | | (m, 1H), 1.48 (s, 4H), 1.23 (s, 16H), |
| | | | | 0.93 (s, 8H), 0.83 (s, 1H). |
| 18 | A | (2S,4R)-4-hydroxy-1-((S)-2- | 807.3 | 1H NMR (400 MHz, DMSO-d6) δ |
| | | (6-(3-(3-(2-hydroxyphenyl)- | | 14.06 (s, 1H), 12.75 (s, 1H), 8.98 (s, |
| | | 7H-pyrrolo[2,3-c]pyridazin-6- | | 1H), 8.61-8.53 (m, 2H), 8.04 (d, J = |
| | | yl)azetidin-1-yl)-6- | | 7.9 Hz, 1H), 7.89 (d, J = 9.3 Hz, |
| | | oxohexanamido)-3,3- | | 1H), 7.40 (q, J = 8.2 Hz, 4H), 7.34- |
| | | dimethylbutanoyl)-N-(4-(4- | | 7.25 (m, 1H), 6.96 (dd, J = 10.5, 7.9 |
| | | methylthiazol-5- | | Hz, 2H), 6.66 (s, 1H), 5.14 (s, 1H), |
| | | yl)benzyl)pyrrolidine-2- | | 4.55 (dt, J = 8.9, 4.0 Hz, 2H), 4.48- |
| | | carboxamide | | 4.38 (m, 2H), 4.38-4.10 (m, 5H), |
| | | | | 4.06 (dd, J = 8.9, 6.2 Hz, 1H), 3.66 |
| | | | | (d, J = 4.6 Hz, 2H), 2.44 (s, 3H), |
| | | | | 2.28 (dd, J = 13.5, 7.2 Hz, 1H), 2.19- |
| | | | | 1.98 (m, 4H), 1.90 (ddd, J = 12.9, |
| | | | | 8.6, 4.5 Hz, 1H), 1.50 (h, J = 7.0 |
| | | | | Hz, 4H), 0.93 (s, 9H). |
| 19 | A | (2S,4R)-4-hydroxy-1-((S)-2- | 975.4 | 1H NMR (300 MHz, DMSO-d6) δ |
| | | (18-(3-(3-(2-hydroxyphenyl)- | | 14.06 (s, 1H), 12.74 (s, 1H), 8.99 (s, |
| | | 7H-pyrrolo[2,3-c]pyridazin-6- | | 1H), 8.63-8.52 (m, 2H), 8.04 (dd, |
| | | yl)azetidin-1-yl)-18- | | J = 8.0, 1.6 Hz, 1H), 7.85 (d, J = 9.3 |
| | | oxooctadecanamido)-3,3- | | Hz, 1H), 7.47-7.35 (m, 4H), 7.30 |
| | | dimethylbutanoyl)-N-(4-(4- | | (ddd, J = 8.4, 7.1, 1.6 Hz, 1H), 7.01- |
| | | methylthiazol-5- | | 6.91 (m, 2H), 6.66 (d, J = 1.4 Hz, |
| | | yl)benzyl)pyrrolidine-2- | | 1H), 5.13 (d, J = 3.5 Hz, 1H), 4.59- |
| | | carboxamide | | 4.49 (m, 2H), 4.49-4.38 (m, 2H), |
| | | | | 4.38-4.01 (m, 6H), 3.66 (d, J = 4.4 |
| | | | | Hz, 2H), 2.45 (s, 3H), 2.31-2.20 |
| | | | | (m, 1H), 2.09 (t, J = 7.5 Hz, 4H), |
| | | | | 1.97-1.83 (m, 1H), 1.48 (d, J = 7.2 |
| | | | | Hz, 4H), 1.23 (d, J = 4.5 Hz, 24H), |
| | | | | 0.94 (s, 9H). |
| 20 | A | (2S,4R)-4-hydroxy-1-((S)-2- | 933.2 | 1H NMR (400 MHz, DMSO-d6) δ |
| | | (15-(3-(3-(2-hydroxyphenyl)- | | 8.98 (s, 1H), 8.58 (d, J = 14.1 Hz, |
| | | 7H-pyrrolo[2,3-c]pyridazin-6- | | 2H), 8.10-7.88 (m, 1H), 7.83 (d, J = |
| | | yl)azetidin-1-yl)-15- | | 9.3 Hz, 1H), 7.40 (q, J = 8.1 Hz, |
| | | oxopentadecanamido)-3,3- | | 5H), 6.98 (s, 2H), 6.71 (s, 1H), 4.54 |
| | | dimethylbutanoyl)-N-(4-(4- | | (d, J = 8.9 Hz, 2H), 4.42 (t, J = 8.1 |
| | | methylthiazol-5- | | Hz, 2H), 4.37-4.11 (m, 5H), 4.07 |
| | | yl)benzyl)pyrrolidine-2- | | (d, J = 7.3 Hz, 1H), 3.65 (s, 2H), |
| | | carboxamide | | 3.51 (s, 1H), 2.44 (s, 3H), 2.33 (s, |
| | | | | 1H), 2.09 (d, J = 7.6 Hz, 3H), 2.06 |
| | | | | (d, J = 5.3 Hz, 1H), 1.89 (s, 1H), |
| | | | | 1.48 (s, 4H), 1.22 (s, 18H), 0.93 (s, |
| | | | | 9H). |
| 21 | A | (2S,4R)-4-hydroxy-1-((S)-2- | 947.4 | 1H NMR (300 MHz, DMSO-d6) δ |
| | | (16-(3-(3-(2-hydroxyphenyl)- | | 14.06 (s, 1H), 12.74 (s, 1H), 8.99 (s, |
| | | 7H-pyrrolo[2,3-c]pyridazin-6- | | 1H), 8.64-8.52 (m, 2H), 8.05 (dd, |
| | | yl)azetidin-1-yl)-16- | | J = 8.2, 1.6 Hz, 1H), 7.85 (d, J = 9.3 |
| | | oxohexadecanamido)-3,3- | | Hz, 1H), 7.46-7.35 (m, 4H), 7.34- |
| | | dimethylbutanoyl)-N-(4-(4- | | 7.24 (m, 1H), 7.02-6.91 (m, 2H), |
| | | methylthiazol-5- | | 6.66 (s, 1H), 5.13 (d, J = 3.5 Hz, |
| | | yl)benzyl)pyrrolidine-2- | | 1H), 4.55 (dt, J = 8.5, 3.7 Hz, 2H), |
| | | carboxamide | | 4.49-4.39 (m, 2H), 4.38-4.22 (m, |
| | | | | 4H), 4.21-4.12 (m, 1H), 4.10- |
| | | | | 4.01 (m, 1H), 3.66 (d, J = 4.5 Hz, |
| | | | | 2H), 2.45 (s, 3H), 2.31-2.19 (m, |
| | | | | 1H), 2.08 (t, J = 6.3, 5.3 Hz, 3H), |
| | | | | 1.99-1.80 (m, 1H), 1.48 (s, 4H), |
| | | | | 1.23 (s, 20H), 0.94 (s, 9H). |
| 22 | A | (2S,4R)-4-hydroxy-1-((S)-2- | 961.2 | 1H NMR (300 MHz, DMSO-d6) δ |
| | | (17-(3-(3-(2-hydroxyphenyl)- | | 14.07 (s, 1H), 12.75 (s, 1H), 8.99 (s, |
| | | 7H-pyrrolo[2,3-c]pyridazin-6- | | 1H), 8.62-8.53 (m, 2H), 8.04 (d, J = |
| | | yl)azetidin-1-yl)-17- | | 7.9 Hz, 1H), 7.85 (d, J = 9.3 Hz, |
| | | oxoheptadecanamido)-3,3- | | 1H), 7.47-7.34 (m, 4H), 7.30 (ddd, |
| | | dimethylbutanoyl)-N-(4-(4- | | J = 8.4, 7.1, 1.6 Hz, 1H), 7.02- |
| | | methylthiazol-5- | | 6.90 (m, 2H), 6.67 (d, J = 1.7 Hz, |
| | | yl)benzyl)pyrrolidine-2- | | 1H), 5.13 (s, 1H), 4.59-4.50 (m, |
| | | carboxamide | | 2H), 4.49-4.39 (m, 2H), 4.38- |
| | | | | 4.12 (m, 5H), 4.11-4.07 (m, 1H), |
| | | | | 3.66 (d, J = 4.3 Hz, 2H), 2.45 (s, |
| | | | | 3H), 2.31-2.20 (m, 1H), 2.08 (t, J = |
| | | | | 6.3, 5.2 Hz, 4H), 2.00-1.83 (m, |
| | | | | 1H), 1.49 (s, 4H), 1.23 (d, J = 4.8 |
| | | | | Hz, 22H), 0.94 (s, 9H). |
| 23 | A | 2-(2,6-dioxopiperidin-3-yl)-4- | 683.3 | 1H NMR (400 MHz, DMSO-d6) δ |
| | | (2-(2-(3-(3-(3-(2- | | 14.05 (s, 1H), 12.70 (s, 1H), 11.11 |
| | | hydroxyphenyl)-7H- | | (s, 1H), 8.56 (s, 1H), 8.02 (dd, J = |
| | | pyrrolo[2,3-c]pyridazin-6- | | 8.1, 1.7 Hz, 1H), 7.78 (t, J = 7.9 Hz, |
| | | yl)azetidin-1-yl)-3- | | 1H), 7.50 (d, J = 8.6 Hz, 1H), 7.42 |
| | | oxopropoxy)ethoxy)ethoxy)isoindoline- | | (d, J = 7.2 Hz, 1H), 7.33-7.25 (m, |
| | | 1,3-dione | | 1H), 7.00-6.91 (m, 2H), 6.64 (s, |
| | | | | 1H), 5.08 (dd, J = 12.8, 5.4 Hz, 1H), |
| | | | | 4.57 (t, J = 8.6 Hz, 1H), 4.39-4.23 |
| | | | | (m, 4H), 4.19-4.02 (m, 2H), 3.82- |
| | | | | 3.75 (m, 2H), 3.68-3.59 (m, 4H), |
| | | | | 3.53 (dd, J = 5.9, 3.6 Hz, 2H), 2.94- |
| | | | | 2.81 (m, 1H), 2.63-2.54 (m, 2H), |
| | | | | 2.35 (d, J = 6.5 Hz, 2H), 2.10-1.94 |
| | | | | (m, 1H). |
| 24 | A | 2-(2,6-dioxopiperidin-3-yl)-5- | 683.2 | 1H NMR (400 MHz, DMSO-d6) δ |
| | | (2-(2-(3-(3-(3-(2- | | 14.05 (s, 1H), 12.72 (s, 1H), 11.12 |
| | | hydroxyphenyl)-7H- | | (s, 1H), 8.57 (s, 1H), 8.02 (d, J = |
| | | pyrrolo[2,3-c]pyridazin-6- | | 7.9 Hz, 1H), 7.79 (d, J = 8.3 Hz, |
| | | yl)azetidin-1-yl)-3- | | 1H), 7.42 (d, J = 2.2 Hz, 1H), 7.37- |
| | | oxopropoxy)ethoxy)ethoxy)isoindoline- | | 7.25 (m, 2H), 7.00-6.90 (m, 2H), |
| | | 1,3-dione | | 6.64 (s, 1H), 5.11 (dd, J = 12.9, 5.3 |
| | | | | Hz, 1H), 4.58 (t, J = 8.5 Hz, 1H), |
| | | | | 4.36 (dd, J = 8.4, 6.0 Hz, 1H), 4.33- |
| | | | | 4.24 (m, 3H), 4.15 (q, J = 7.1 Hz, |
| | | | | 1H), 4.07 (dd, J = 8.9, 6.2 Hz, 1H), |
| | | | | 3.77 (t, J = 4.4 Hz, 2H), 3.68-3.56 |
| | | | | (m, 4H), 3.54 (dd, J = 6.0, 3.6 Hz, |
| | | | | 2H), 2.95-2.81 (m, 1H), 2.62- |
| | | | | 2.53 (m, 4H), 2.35 (t, J = 6.6 Hz, |
| | | | | 2H), 2.04 (dd, J = 10.6, 5.2 Hz, 1H). |
| 25 | A | 2-(2,6-dioxopiperidin-3-yl)-5- | 678.2 | 1H NMR (400 MHz, DMSO-d6) δ |
| | | (2-(4-(3-(3-(2- | | 14.05 (s, 1H), 12.77 (s, 1H), 11.12 |
| | | hydroxyphenyl)-7H- | | (s, 1H), 8.59 (s, 1H), 8.22 (s, 1H, |
| | | pyrrolo[2,3-c]pyridazin-6- | | FA), 8.04 (dd, J = 8.0, 1.7 Hz, 1H), |
| | | yl)azetidine-1- | | 7.83 (d, J = 8.3 Hz, 1H), 7.47 (d, J = |
| | | carbonyl)piperidin-1- | | 2.3 Hz, 1H), 7.37 (dd, J = 8.4, 2.3 |
| | | yl)ethoxy)isoindoline-1,3- | | Hz, 1H), 7.29 (ddd, J = 8.4, 7.1, 1.6 |
| | | dione | | Hz, 1H), 7.00-6.91 (m, 2H), 6.67 |
| | | | | (s, 1H), 5.12 (dd, J = 12.9, 5.4 Hz, |
| | | | | 1H), 4.61 (t, J = 8.6 Hz, 1H), 4.41 |
| | | | | (dd, J = 8.4, 6.1 Hz, 1H), 4.29 (q, J = |
| | | | | 8.8, 7.1 Hz, 3H), 4.23-4.11 (m, |
| | | | | 1H), 4.06 (dd, J = 9.1, 6.1 Hz, 1H), |
| | | | | 3.02-2.95 (m, 2H), 2.95-2.82 (m, |
| | | | | 1H), 2.74 (t, J = 5.7 Hz, 2H), 2.64- |
| | | | | 2.54 (m, 2H), 2.30-2.20 (m, 1H), |
| | | | | 2.08 (dd, J = 15.9, 8.8 Hz, 3H), 1.75- |
| | | | | 1.47 (m, 4H). |
| 26 | A | 2-(2,6-dioxopiperidin-3-yl)-5- | 663.2 | 1H NMR (400 MHz, DMSO-d6) δ |
| | | (4-(3-(3-(3-(2- | | 14.04 (s, 1H), 12.75 (s, 1H), 11.09 |
| | | hydroxyphenyl)-7H- | | (s, 1H), 8.58 (s, 1H), 8.14 (s, 1H, |
| | | pyrrolo[2,3-c]pyridazin-6- | | FA), 8.02 (dd, J = 8.1, 1.7 Hz, 1H), |
| | | yl)azetidin-1-yl)-3- | | 7.67 (d, J = 8.4 Hz, 1H), 7.38-7.19 |
| | | oxopropyl)piperazin-1- | | (m, 3H), 7.00-6.91 (m, 2H), 6.67 |
| | | yl)isoindoline-1,3-dione | | (s, 1H), 5.07 (dd, J = 13.0, 5.3 Hz, |
| | | | | 1H), 4.61 (t, J = 8.5 Hz, 1H), 4.42- |
| | | | | 4.26 (m, 2H), 4.23-4.02 (m, 2H), |
| | | | | 3.49-3.38 (m, 4H), 3.32-3.28 (m, |
| | | | | 6H), 2.95-2.82 (m, 1H), 2.63- |
| | | | | 2.52 (m, 3H), 2.39-2.34 (m, 1H), |
| | | | | 2.02 (d, J = 12.4 Hz, 1H). |
| 27 | A | 2-(2,6-dioxopiperidin-3-yl)-4- | 682.2 | 1H NMR (400 MHz, DMSO-d6) δ |
| | | ((2-(2-(3-(3-(3-(2- | | 14.06 (s, 1H), 12.71 (s, 1H), 11.10 |
| | | hydroxyphenyl)-7H- | | (s, 1H), 8.56 (s, 1H), 8.03 (dd, J = |
| | | pyrrolo[2,3-c]pyridazin-6- | | 8.0, 1.6 Hz, 1H), 7.55 (t, J = 7.9 Hz, |
| | | yl)azetidin-1-yl)-3- | | 1H), 7.29 (td, J = 8.3, 7.7, 1.6 Hz, |
| | | oxopropoxy)ethoxy)ethyl)amino)isoindoline- | | 1H), 7.11 (d, J = 8.6 Hz, 1H), 7.01 |
| | | 1,3-dione | | (d, J = 7.0 Hz, 1H), 6.95 (dd, J = |
| | | | | 10.7, 7.9 Hz, 2H), 6.63 (s, 1H), 6.59 |
| | | | | (t, J = 5.8 Hz, 1H), 5.05 (dd, J = |
| | | | | 13.0, 5.4 Hz, 1H), 4.56 (t, J = 8.6 |
| | | | | Hz, 1H), 4.39-4.22 (m, 2H), 4.19- |
| | | | | 4.01 (m, 2H), 3.67-3.49 (m, 8H), |
| | | | | 3.44 (q, J = 5.5 Hz, 2H), 2.94-2.80 |
| | | | | (m, 1H), 2.62-2.53 (m, 2H), 2.34 |
| | | | | (t, J = 6.5 Hz, 2H), 2.02 (d, J = 12.9 |
| | | | | Hz, 1H). |
| 28 | A | 2-(2,6-dioxopiperidin-3-yl)-4- | 706.3 | 1H NMR (400 MHz, DMSO-d6) δ |
| | | ((11-(3-(3-(2- | | 14.06 (s, 1H), 12.74 (s, 1H), 11.10 |
| | | hydroxyphenyl)-7H- | | (s, 1H), 8.59 (s, 1H), 8.04 (dd, J = |
| | | pyrrolo[2,3-c]pyridazin-6- | | 8.0, 1.6 Hz, 1H), 7.57 (dd, J = 8.6, |
| | | yl)azetidin-1-yl)-11- | | 7.1 Hz, 1H), 7.29 (ddd, J = 8.4, 7.0, |
| | | oxoundecyl)amino)isoindoline- | | 1.6 Hz, 1H), 7.08 (d, J = 8.6 Hz, |
| | | 1,3-dione | | 1H), 7.03-6.91 (m, 3H), 6.65 (s, |
| | | | | 1H), 6.52 (t, J = 6.0 Hz, 1H), 5.05 |
| | | | | (dd, J = 12.8, 5.4 Hz, 1H), 4.54 (t, J = |
| | | | | 8.5 Hz, 1H), 4.37-4.23 (m, 2H), |
| | | | | 4.15 (ddd, J = 14.7, 8.7, 6.1 Hz, |
| | | | | 1H), 4.06 (dd, J = 9.0, 6.1 Hz, 1H), |
| | | | | 3.28 (q, J = 7.6, 6.7 Hz, 2H), 2.95- |
| | | | | 2.81 (m, 1H), 2.63-2.50 (m, 2H), |
| | | | | 2.15-1.93 (m, 3H), 1.60-1.43 (m, |
| | | | | 4H), 1.32-1.21 (m, 12H). |
| 29 | A | 2-(2,6-dioxopiperidin-3-yl)-5- | 682.2 | 1H NMR (400 MHz, DMSO-d6) δ |
| | | ((2-(2-(3-(3-(3-(2- | | 14.06 (s, 1H), 12.71 (s, 1H), 11.10 |
| | | hydroxyphenyl)-7H- | | (s, 1H), 8.57 (s, 1H), 8.03 (dd, J = |
| | | pyrrolo[2,3-c]pyridazin-6- | | 8.0, 1.6 Hz, 1H), 7.59-7.51 (m, |
| | | yl)azetidin-1-yl)-3- | | 1H), 7.29 (ddd, J = 8.5, 7.1, 1.6 Hz, |
| | | oxopropoxy)ethoxy)ethyl)amino)isoindoline- | | 1H), 7.11 (d, J = 8.6 Hz, 1H), 7.01 |
| | | 1,3-dione | | (d, J = 7.1 Hz, 1H), 7.00-6.91 (m, |
| | | | | 2H), 6.63 (s, 1H), 6.59 (t, J = 5.8 |
| | | | | Hz, 1H), 5.05 (dd, J = 13.0, 5.4 Hz, |
| | | | | 1H), 4.56 (t, J = 8.6 Hz, 1H), 4.39- |
| | | | | 4.23 (m, 2H), 4.19-4.02 (m, 2H), |
| | | | | 3.67-3.58 (m, 4H), 3.57-3.49 (m, |
| | | | | 4H), 3.44 (q, J = 5.6 Hz, 2H), 2.94- |
| | | | | 2.80 (m, 1H), 2.63-2.51 (m, 2H), |
| | | | | 2.34 (t, J = 6.5 Hz, 2H), 2.07-1.97 |
| | | | | (m, 1H). |
| 30 | A | 2-(2,6-dioxopiperidin-3-yl)-4- | 665.2 | 1H NMR (400 MHz, DMSO-d6) δ |
| | | ((8-(3-(3-(2-hydroxyphenyl)- | | 14.06 (s, 1H), 12.75 (s, 1H), 11.11 |
| | | 7H-pyrrolo[2,3-c]pyridazin-6- | | (s, 1H), 8.59 (s, 1H), 8.03 (dd, J = |
| | | yl)azetidin-1-yl)-8- | | 8.0, 1.7 Hz, 1H), 7.80 (dd, J = 8.5, |
| | | oxooctyl)oxy)isoindoline-1,3- | | 7.2 Hz, 1H), 7.51 (d, J = 8.6 Hz, |
| | | dione | | 1H), 7.43 (d, J = 7.2 Hz, 1H), 7.30 |
| | | | | (ddd, J = 8.4, 7.0, 1.6 Hz, 1H), 7.01- |
| | | | | 6.91 (m, 2H), 6.66 (d, J = 1.8 Hz, |
| | | | | 1H), 5.08 (dd, J = 12.8, 5.4 Hz, 1H), |
| | | | | 4.54 (t, J = 8.5 Hz, 1H), 4.38-4.24 |
| | | | | (m, 2H), 4.23-4.11 (m, 3H), 4.14- |
| | | | | 4.02 (m, 1H), 2.95-2.81 (m, 1H), |
| | | | | 2.63-2.53 (m, 2H), 2.11 (t, J = 7.4 |
| | | | | Hz, 2H), 2.07-1.98 (m, 1H), 1.76 |
| | | | | (p, J = 6.6 Hz, 2H), 1.58-1.40 (m, |
| | | | | 4H), 1.37-1.27 (m, 4H). |
| 31 | A | 2-(2,6-dioxopiperidin-3-yl)-4- | 651.2 | 1H NMR (400 MHz, DMSO-d6) δ |
| | | ((7-(3-(3-(2-hydroxyphenyl)- | | 13.15 (s, 1H), 11.11 (s, 1H), 8.60 (s, |
| | | 7H-pyrrolo[2,3-c]pyridazin-6- | | 1H), 7.89-7.76 (m, 2H), 7.52 (d, J = |
| | | yl)azetidin-1-yl)-7- | | 8.5 Hz, 1H), 7.44 (d, J = 7.2 Hz, |
| | | oxoheptyl)oxy)isoindoline- | | 1H), 7.37 (t, J = 7.8 Hz, 1H), 7.06- |
| | | 1,3-dione | | 6.94 (m, 2H), 6.81 (s, 1H), 5.08 (dd, |
| | | | | J = 12.9, 5.4 Hz, 1H), 4.57 (t, J = |
| | | | | 8.6 Hz, 1H), 4.40-4.26 (m, 2H), |
| | | | | 4.21 (t, J = 6.4 Hz, 3H), 4.08 (dd, J = |
| | | | | 9.0, 5.9 Hz, 1H), 2.94-2.80 (m, |
| | | | | 1H), 2.57 (dd, J = 19.8, 6.4 Hz, 2H), |
| | | | | 2.12 (t, J = 7.4 Hz, 2H), 2.08-1.98 |
| | | | | (m, 1H), 1.77 (p, J = 6.6 Hz, 2H), |
| | | | | 1.50 (dq, J = 23.5, 7.3 Hz, 4H), 1.43- |
| | | | | 1.33 (m, 2H). |
| 32 | A | 2-(2,6-dioxopiperidin-3-yl)-5- | 706.1 | 1H NMR (400 MHz, DMSO-d6) δ |
| | | ((11-(3-(3-(2- | | 14.06 (s, 1H), 12.74 (s, 1H), 11.06 |
| | | hydroxyphenyl)-7H- | | (s, 1H), 8.59 (s, 1H), 8.04 (dd, J = |
| | | pyrrolo[2,3-c]pyridazin-6- | | 8.1, 1.7 Hz, 1H), 7.55 (d, J = 8.3 |
| | | yl)azetidin-1-yl)-11- | | Hz, 1H), 7.29 (ddd, J = 8.5, 7.1, 1.6 |
| | | oxoundecyl)amino)isoindoline- | | Hz, 1H), 7.10 (t, J = 5.4 Hz, 1H), |
| | | 1,3-dione | | 7.00-6.91 (m, 3H), 6.83 (dd, J = |
| | | | | 8.4, 2.1 Hz, 1H), 6.66 (d, J = 1.8 |
| | | | | Hz, 1H), 5.03 (dd, J = 12.9, 5.3 Hz, |
| | | | | 1H), 4.54 (t, J = 8.6 Hz, 1H), 4.39- |
| | | | | 4.23 (m, 2H), 4.21-4.10 (m, 1H), |
| | | | | 4.06 (dd, J = 8.9, 6.1 Hz, 1H), 3.14 |
| | | | | (q, J = 6.6 Hz, 2H), 2.87 (ddd, J = |
| | | | | 17.2, 14.0, 5.4 Hz, 1H), 2.61-2.51 |
| | | | | (m, 2H), 2.09 (t, J = 7.4 Hz, 2H), |
| | | | | 2.04-1.92 (m, 1H), 1.60-1.47 (m, |
| | | | | 4H), 1.41-1.15 (m, 12H). |
| 33 | A | 2-(2,6-dioxopiperidin-3-yl)-4- | 678.1 | 1H NMR (400 MHz, DMSO-d6) δ |
| | | ((9-(3-(3-(2-hydroxyphenyl)- | | 14.06 (s, 1H), 12.74 (s, 1H), 11.10 |
| | | 7H-pyrrolo[2,3-c]pyridazin-6- | | (s, 1H), 8.59 (s, 1H), 8.04 (dd, J = |
| | | yl)azetidin-1-yl)-9- | | 8.1, 1.7 Hz, 1H), 7.57 (dd, J = 8.6, |
| | | oxononyl)amino)isoindoline- | | 7.1 Hz, 1H), 7.33-7.25 (m, 1H), |
| | | 1,3-dione | | 7.08 (d, J = 8.6 Hz, 1H), 7.04-6.90 |
| | | | | (m, 3H), 6.66 (d, J = 1.8 Hz, 1H), |
| | | | | 6.52 (t, J = 5.6 Hz, 1H), 5.05 (dd, J = |
| | | | | 12.9, 5.3 Hz, 1H), 4.54 (t, J = 8.5 |
| | | | | Hz, 1H), 4.37-4.23 (m, 2H), 4.15 |
| | | | | (p, J = 7.8, 7.0 Hz, 1H), 4.06 (dd, J = |
| | | | | 9.0, 6.1 Hz, 1H), 3.27 (t, J = 6.8 |
| | | | | Hz, 2H), 2.95-2.81 (m, 1H), 2.63- |
| | | | | 2.53 (m, 2H), 2.09 (t, J = 7.4 Hz, |
| | | | | 2H), 2.06-1.98 (m, 1H), 1.67- |
| | | | | 1.43 (m, 4H), 1.37-1.22 (m, 8H). |
| 34 | A | 2-(2,6-dioxopiperidin-3-yl)-5- | 675.2 | 1H NMR (400 MHz, DMSO-d6) δ |
| | | (6-(3-(3-(3-(2- | | 14.06 (s, 1H), 12.75 (s, 1H), 11.07 |
| | | hydroxyphenyl)-7H- | | (s, 1H), 8.60 (s, 1H), 8.27 (s, 1H, |
| | | pyrrolo[2,3-c]pyridazin-6- | | FA), 8.04 (dd, J = 8.0, 1.6 Hz, 1H), |
| | | yl)azetidin-1-yl)-3- | | 7.60 (d, J = 8.3 Hz, 1H), 7.33-7.25 |
| | | oxopropyl)-2,6- | | (m, 1H), 6.95 (td, J = 8.3, 2.5 Hz, |
| | | diazaspiro[3.3]heptan-2- | | 2H), 6.78 (d, J = 2.1 Hz, 1H), 6.67 |
| | | yl)isoindoline-1,3-dione | | (s, 1H), 6.61 (dd, J = 8.3, 2.1 Hz, |
| | | | | 1H), 5.05 (dd, J = 12.9, 5.3 Hz, 1H), |
| | | | | 4.57 (t, J = 8.6 Hz, 1H), 4.36 (dd, J = |
| | | | | 8.4, 5.9 Hz, 1H), 4.29 (t, J = 8.9 |
| | | | | Hz, 1H), 4.21-4.10 (m, 1H), 4.15- |
| | | | | 4.01 (m, 5H), 3.30 (s, 4H), 2.94- |
| | | | | 2.81 (m, 1H), 2.63-2.53 (m, 4H), |
| | | | | 2.15-2.06 (m, 2H), 2.04-1.96 (m, |
| | | | | 1H). |
| 35 | A | (2S,4R)-4-hydroxy-1-((S)-2- | 751.4 | 1H NMR (400 MHz, DMSO-d6) δ |
| | | (3-(3-(3-(2-hydroxyphenyl)- | | 14.03 (s, 1H), 12.46 (s, 1H), 8.95 (s, |
| | | 7H-pyrrolo[2,3-c]pyridazin-5- | | 1H), 8.74 (s, 1H), 8.57 (t, J = 6.1 |
| | | yl)azetidin-1- | | Hz, 1H), 8.31 (d, J = 9.3 Hz, 1H), |
| | | yl)propanamido)-3,3- | | 8.21 (s, 1H, FA), 8.09 (dd, J = 8.3, |
| | | dimethylbutanoyl)-N-(4-(4- | | 1.7 Hz, 1H), 8.01 (s, 1H), 7.45- |
| | | methylthiazol-5- | | 7.34 (m, 4H), 7.29 (td, J = 7.6, 1.6 |
| | | yl)benzyl)pyrrolidine-2- | | Hz, 1H), 7.00-6.93 (m, 2H), 5.15 |
| | | carboxamide | | (s, 1H), 4.55 (d, J = 9.4 Hz, 1H), |
| | | | | 4.48-4.39 (m, 2H), 4.36 (t, J = 3.5 |
| | | | | Hz, 1H), 4.24 (dd, J = 15.8, 5.5 Hz, |
| | | | | 1H), 3.93-3.86 (m, 1H), 3.73 (t, J = |
| | | | | 6.7 Hz, 2H), 3.67 (d, J = 3.2 Hz, |
| | | | | 2H), 3.25 (s, 2H), 2.77-2.65 (m, |
| | | | | 2H), 2.42 (s, 3H), 2.32 (s, 1H), 2.27- |
| | | | | 2.20 (m, 1H), 2.08-2.00 (m, 1H), |
| | | | | 1.95-1.87 (m, 1H), 0.94 (s, 9H). |
| 36 | A | (2S,4R)-4-hydroxy-1-((S)-2- | 849.5 | 1H NMR (400 MHz, DMSO-d6) δ |
| | | (9-(3-(3-(2-hydroxyphenyl)- | | 13.88 (s, 1H), 12.50 (s, 1H), 8.98 (s, |
| | | 7H-pyrrolo[2,3-c]pyridazin-5- | | 1H), 8.58 (d, J = 18.4 Hz, 2H), 8.12 |
| | | yl)azetidin-1-yl)-9- | | (s, 1H), 8.05 (dd, J = 8.3, 1.6 Hz, |
| | | oxononanamido)-3,3- | | 1H), 7.84 (d, J = 9.4 Hz, 1H), 7.40 |
| | | dimethylbutanoyl)-N-(4-(4- | | (q, J = 8.3 Hz, 4H), 7.30 (ddd, J = |
| | | methylthiazol-5- | | 8.6, 7.2, 1.6 Hz, 1H), 6.97 (t, J = 7.6 |
| | | yl)benzyl)pyrrolidine-2- | | Hz, 2H), 5.12 (d, J = 3.5 Hz, 1H), |
| | | carboxamide | | 4.64-4.51 (m, 2H), 4.48-4.39 (m, |
| | | | | 2H), 4.37-4.30 (m, 2H), 4.28- |
| | | | | 4.18 (m, 2H), 4.17-4.09 (m, 1H), |
| | | | | 4.06-3.99 (m, 1H), 3.72-3.60 (m, |
| | | | | 2H), 2.44 (s, 3H), 2.27-2.21 (m, |
| | | | | 1H), 2.11 (q, J = 8.0, 7.6 Hz, 3H), |
| | | | | 2.01 (d, J = 8.6 Hz, 1H), 1.90 (ddd, |
| | | | | J = 12.9, 8.6, 4.6 Hz, 1H), 1.55- |
| | | | | 1.42 (m, 4H), 1.31-1.18 (m, 6H), |
| | | | | 0.93 (s, 9H). |
| 37 | A | (2S,4R)-4-hydroxy-1-((S)-2- | 876.9 | 1H NMR (400 MHz, DMSO-d6) δ |
| | | (11-(3-(3-(2-hydroxyphenyl)- | | 13.89 (s, 1H), 12.40 (s, 1H), 8.98 (s, |
| | | 7H-pyrrolo[2,3-c]pyridazin-5- | | 1H), 8.63-8.59 (m, 1H), 8.58- |
| | | yl)azetidin-1-yl)-11- | | 8.54 (m, 1H), 8.12 (s, 1H), 8.05 (dd, |
| | | oxoundecanamido)-3,3- | | J = 8.0, 1.7 Hz, 1H), 7.84 (d, J = 9.3 |
| | | dimethylbutanoyl)-N-(4-(4- | | Hz, 1H), 7.40 (q, J = 8.4 Hz, 4H), |
| | | methylthiazol-5- | | 7.34-7.26 (m, 1H), 7.02-6.93 (m, |
| | | yl)benzyl)pyrrolidine-2- | | 2H), 5.14 (s, 1H), 4.61-4.52 (m, |
| | | carboxamide | | 2H), 4.46-4.39 (m, 2H), 4.33 (t, J = |
| | | | | 8.7 Hz, 2H), 4.28-4.18 (m, 2H), |
| | | | | 4.17-4.08 (m, 1H), 4.06-3.99 (m, |
| | | | | 1H), 3.66 (d, J = 5.0 Hz, 2H), 2.44 |
| | | | | (s, 3H), 2.26 (dd, J = 14.3, 7.3 Hz, |
| | | | | 1H), 2.13-2.05 (m, 3H), 2.04- |
| | | | | 1.98 (m, 1H), 1.90 (ddd, J = 12.8, |
| | | | | 8.4, 4.6 Hz, 1H), 1.49 (d, J = 7.4 |
| | | | | Hz, 4H), 1.23 (s, 10H), 0.93 (s, 9H). |
| 38 | A | (2S,4R)-4-hydroxy-1-((S)-2- | 974.9 | 1H NMR (400 MHz, DMSO-d6) δ |
| | | (18-(3-(3-(2-hydroxyphenyl)- | | 13.90 (s, 1H), 12.45 (s, 1H), 8.98 (s, |
| | | 7H-pyrrolo[2,3-c]pyridazin-5- | | 1H), 8.62-8.59 (m, 1H),8.58- |
| | | yl)azetidin-1-yl)-18- | | 8.54 (m, 1H), 8.11 (s, 1H), 8.04 |
| | | oxooctadecanamido)-3,3- | | (dd, J = 8.2, 1.7 Hz, 1H), 7.84 (d, J = |
| | | dimethylbutanoyl)-N-(4-(4- | | 9.2 Hz, 1H), 7.40 (q, J = 8.2 Hz, |
| | | methylthiazol-5- | | 4H), 7.34-7.26 (m, 1H), 7.01- |
| | | yl)benzyl)pyrrolidine-2- | | 6.92 (m, 2H), 5.14 (s, 1H), 4.62- |
| | | carboxamide | | 4.52 (m, 2H), 4.43 (q, J = 7.3 Hz, |
| | | | | 2H), 4.33 (t, J = 8.7 Hz, 2H), 4.29- |
| | | | | 4.16 (m, 2H), 4.12 (t, J = 7.2 Hz, |
| | | | | 1H), 4.07-3.98 (m, 1H), 3.65 (d, J = |
| | | | | 6.1 Hz, 2H), 2.44 (s, 3H), 2.25 |
| | | | | (dd, J = 14.7, 7.2 Hz, 1H), 2.13- |
| | | | | 2.05 (m, 3H), 2.04-1.99 (m, 1H), |
| | | | | 1.95-1.84 (m, 1H), 1.49 (d, J = 7.3 |
| | | | | Hz, 4H), 1.21 (s, 24H), 0.93 (s, 9H). |
| 39 | A | (2S,4R)-4-hydroxy-1-((S)-2- | 932.9 | 1H NMR (400 MHz, DMSO-d6) δ |
| | | (15-(3-(3-(2-hydroxyphenyl)- | | 13.89 (s, 1H), 12.45 (s, 1H),8.98 (s, |
| | | 7H-pyrrolo[2,3-c]pyridazin-5- | | 1H), 8.62-8.59 (m, 1H), 8.58- |
| | | yl)azetidin-1-yl)-15- | | 8.53 (m, 1H), 8.11 (s, 1H), 8.04 (dd, |
| | | oxopentadecanamido)-3,3- | | J = 8.0, 1.7 Hz, 1H), 7.84 (d, J = 9.3 |
| | | dimethylbutanoyl)-N-(4-(4- | | Hz, 1H), 7.40 (q, J = 8.3 Hz, 4H), |
| | | methylthiazol-5- | | 7.33-7.28 (m, 1H), 7.01-6.94 (m, |
| | | yl)benzyl)pyrrolidine-2- | | 2H), 5.13 (s, 1H), 4.64-4.52 (m, |
| | | carboxamide | | 2H), 4.43 (q, J = 7.3 Hz, 2H), 4.33 |
| | | | | (t, J = 8.6 Hz, 2H), 4.28-4.17 (m, |
| | | | | 2H), 4.12 (p, J = 7.9, 7.3 Hz, 1H), |
| | | | | 4.02 (dd, J = 9.1, 6.1 Hz, 1H), 3.65 |
| | | | | (d, J = 6.1 Hz, 2H), 2.44 (s, 3H), |
| | | | | 2.25 (dt, J = 14.7, 7.5 Hz, 1H), 2.13- |
| | | | | 2.06 (m, 3H), 2.05-2.00 (m, 1H), |
| | | | | 1.94-1.86 (m, 1H), 1.49 (d, J = 6.8 |
| | | | | Hz, 4H), 1.21 (s, 18H), 0.93 (s, 9H). |
| 40 | A | (2S,4R)-4-hydroxy-1-((S)-2- | 793.4 | 1H NMR (400 MHz, DMSO-d6) δ |
| | | (5-(3-(3-(2-hydroxyphenyl)- | | 14.04-12.80 (m, 1H), 12.65- |
| | | 7H-pyrrolo[2,3-c]pyridazin-5- | | 12.52 (m, 1H), 8.98 (s, 1H), 8.62 (s, |
| | | yl)azetidin-1-yl)-5- | | 1H), 8.58-8.52 (m, 1H), 8.18- |
| | | oxopentanamido)-3,3- | | 8.12 (m, 1H), 8.08-8.02 (m, 1H), |
| | | dimethylbutanoyl)-N-(4-(4- | | 7.90 (d, J = 9.3 Hz, 1H), 7.44-7.26 |
| | | methylthiazol-5- | | (m, 5H), 7.02-6.94 (m, 2H), 5.18- |
| | | yl)benzyl)pyrrolidine-2- | | 5.06 (m, 1H), 4.63-4.51 (m, 2H), |
| | | carboxamide | | 4.46-4.38 (m, 2H), 4.38-4.30 (m, |
| | | | | 2H), 4.28-4.17 (m, 2H), 4.17- |
| | | | | 4.08 (m, 1H), 4.06-3.98 (m, 1H), |
| | | | | 3.71-3.61 (m, 2H), 2.44 (s, 3H), |
| | | | | 2.31-2.16 (m, 2H), 2.11 (t, J = 7.5 |
| | | | | Hz, 2H), 2.06-1.97 (m, 1H), 1.94- |
| | | | | 1.85 (m, 1H), 1.80-1.68 (m, 2H), |
| | | | | 0.93 (d, J = 5.0 Hz, 9H). |
| 41 | A | (2S,4R)-4-hydroxy-1-((S)-2- | 947.6 | 1H NMR (400 MHz, DMSO-d6) δ |
| | | (16-(3-(3-(2-hydroxyphenyl)- | | 14.00-13.75 (m, 1H), 12.63- |
| | | 7H-pyrrolo[2,3-c]pyridazin-5- | | 12.52 (m, 1H), 8.98 (s, 1H), 8.60 (s, |
| | | yl)azetidin-1-yl)-16- | | 1H), 8.56 (t, J = 6.1 Hz, 1H), 8.14 |
| | | oxohexadecanamido)-3,3- | | (s, 1H), 8.03 (d, J = 7.9 Hz, 1H), |
| | | dimethylbutanoyl)-N-(4-(4- | | 7.83 (d, J = 9.3 Hz, 1H), 7.45-7.35 |
| | | methylthiazol-5- | | (m, 4H), 7.34-7.26 (m, 1H), 7.01- |
| | | yl)benzyl)pyrrolidine-2- | | 6.93 (m, 2H), 5.19-5.06 (m, 1H), |
| | | carboxamide | | 4.60 (t, J = 8.5 Hz, 1H), 4.54 (d, J = |
| | | | | 9.4 Hz, 1H), 4.47-4.38 (m, 2H), |
| | | | | 4.38-4.29 (m, 2H), 4.29-4.17 (m, |
| | | | | 2H), 4.17-4.07 (m, 1H), 4.07- |
| | | | | 3.98 (m, 1H), 3.71-3.60 (m, 2H), |
| | | | | 2.44 (s, 3H), 2.30-2.20 (m, 1H), |
| | | | | 2.15-1.98 (m, 4H), 1.95-1.85 (m, |
| | | | | 1H), 1.56-1.37 (m, 4H), 1.32- |
| | | | | 1.13 (m, 20H), 0.93 (s, 9H). |
| 42 | A | (2S,4R)-4-hydroxy-1-((S)-2- | 961.6 | 1H NMR (400 MHz, DMSO-d6) δ |
| | | (17-(3-(3-(2-hydroxyphenyl)- | | 13.88 (s, 1H), 12.56-12.52 (m, |
| | | 7H-pyrrolo[2,3-c]pyridazin-5- | | 1H), 8.98 (s, 1H), 8.60 (s, 1H), 8.56 |
| | | yl)azetidin-1-yl)-17- | | (t, J = 6.1 Hz, 1H), 8.12 (d, J = 2.5 |
| | | oxoheptadecanamido)-3,3- | | Hz, 1H), 8.04 (dd, J = 7.5, 1.4 Hz, |
| | | dimethylbutanoyl)-N-(4-(4- | | 1H), 7.83 (d, J = 9.3 Hz, 1H), 7.44- |
| | | methylthiazol-5- | | 7.35 (m, 4H), 7.33-7.27 (m, 1H), |
| | | yl)benzyl)pyrrolidine-2- | | 7.00-6.93 (m, 2H), 5.12 (d, J = 3.6 |
| | | carboxamide | | Hz, 1H), 4.60 (t, J = 8.4 Hz, 1H), |
| | | | | 4.54 (d, J = 9.4 Hz, 1H), 4.47-4.39 |
| | | | | (m, 2H), 4.37-4.30 (m, 2H), 4.29- |
| | | | | 4.16 (m, 2H), 4.16-4.08 (m, 1H), |
| | | | | 4.06-3.99 (m, 1H), 3.70-3.60 (m, |
| | | | | 2H), 2.44 (s, 3H), 2.30-2.19 (m, |
| | | | | 1H), 2.14-1.97 (m, 4H), 1.94- |
| | | | | 1.85 (m, 1H), 1.55-1.37 (m, 4H), |
| | | | | 1.32-1.13 (m, 22H), 0.93 (s, 9H). |
| 43 | A | (2S,4R)-4-hydroxy-1-((S)-2- | 863.3 | 1H NMR (400 MHz, DMSO-d6) δ |
| | | (10-(3-(3-(2-hydroxyphenyl)- | | 13.88 (s, 1H), 12.51 (s, 1H), 8.98 (s, |
| | | 7H-pyrrolo[2,3-c]pyridazin-5- | | 1H), 8.60 (s, 1H), 8.56 (t, J = 6.1 |
| | | yl)azetidin-1-yl)-10- | | Hz, 1H), 8.12 (s, 1H), 8.05 (d, J = |
| | | oxodecanamido)-3,3- | | 7.8 Hz, 1H), 7.83 (d, J = 9.3 Hz, |
| | | dimethylbutanoyl)-N-(4-(4- | | 1H), 7.40 (q, J = 8.1 Hz, 4H), 7.35- |
| | | methylthiazol-5- | | 7.26 (m, 1H), 7.01-6.93 (m, 2H), |
| | | yl)benzyl)pyrrolidine-2- | | 5.12 (d, J = 3.5 Hz, 1H), 4.60 (t, J = |
| | | carboxamide | | 8.5 Hz, 1H), 4.54 (d, J = 9.4 Hz, |
| | | | | 1H), 4.48-4.38 (m, 2H), 4.38- |
| | | | | 4.29 (m, 2H), 4.29-4.17 (m, 2H), |
| | | | | 4.17-4.07 (m, 1H), 4.06-3.98 (m, |
| | | | | 1H), 3.68-3.59 (m, 2H), 2.44 (s, |
| | | | | 3H), 2.29-2.19 (m, 1H), 2.16- |
| | | | | 2.04 (m, 3H), 2.01 (d, J = 8.6 Hz, |
| | | | | 1H), 1.95-1.84 (m, 1H), 1.52- |
| | | | | 1.46 (m, 4H), 1.24 (s, 8H), 0.93 (s, |
| | | | | 9H). |
| 44 | A | (2S,4R)-4-hydroxy-1-((S)-2- | 919.3 | 1H NMR (400 MHz, DMSO-d6) δ |
| | | (14-(3-(3-(2-hydroxyphenyl)- | | 13.89 (s, 1H), 12.54 (s, 1H), 8.98 (s, |
| | | 7H-pyrrolo[2,3-c]pyridazin-5- | | 1H), 8.60 (s, 1H), 8.58-8.52 (m, |
| | | yl)azetidin-1-yl)-14- | | 1H), 8.12 (s, 1H), 8.04 (d, J = 7.8 |
| | | oxotetradecanamido)-3,3- | | Hz, 1H), 7.83 (d, J = 9.5 Hz, 1H), |
| | | dimethylbutanoyl)-N-(4-(4- | | 7.45-7.35 (m, 4H), 7.30 (t, J = 7.7 |
| | | methylthiazol-5- | | Hz, 1H), 7.01-6.95 (m, 2H), 5.12 |
| | | yl)benzyl)pyrrolidine-2- | | (s, 1H), 4.65-4.57 (m, 1H), 4.54 |
| | | carboxamide | | (d, J = 9.1 Hz, 1H), 4.42 (t, J = 7.9 |
| | | | | Hz, 2H), 4.38-4.29 (m, 2H), 4.26- |
| | | | | 4.22 (m, 2H), 4.15-4.10 (m, 1H), |
| | | | | 4.07-3.99 (m, 1H), 3.65 (s, 2H), |
| | | | | 2.44 (s, 3H), 2.30-2.18 (m, 1H), |
| | | | | 2.14-2.07 (m, 3H), 2.07-1.96 (m, |
| | | | | 1H), 1.96-1.79 (m, 1H), 1.52- |
| | | | | 1.47 (m, 4H), 1.33-1.17 (m, 16H), |
| | | | | 0.93 (s, 9H). |
| 45 | A | (2S,4R)-4-hydroxy-1-((S)-2- | 835.3 | 1H NMR (400 MHz, DMSO-d6) δ |
| | | (8-(3-(3-(2-hydroxyphenyl)- | | 12.68 (s, 1H), 8.98 (s, 1H), 8.62 (s, |
| | | 7H-pyrrolo[2,3-c]pyridazin-5- | | 1H), 8.56 (t, J = 5.9 Hz, 1H), 8.21 |
| | | yl)azetidin-1-yl)-8- | | (s, 1H), 8.00 (d, J = 8.1 Hz, 1H), |
| | | oxooctanamido)-3,3- | | 7.84 (d, J = 9.3 Hz, 1H), 7.40 (q, J = |
| | | dimethylbutanoyl)-N-(4-(4- | | 8.3 Hz, 4H), 7.32 (t, J = 7.7 Hz, 1H), |
| | | methylthiazol-5- | | 7.03-6.94 (m, 2H), 4.65-4.50 (m, |
| | | yl)benzyl)pyrrolidine-2- | | 2H), 4.48-4.38 (m, 2H), 4.37- |
| | | carboxamide | | 4.29 (m, 2H), 4.29-4.08 (m, 3H), |
| | | | | 4.06-3.98 (m, 1H), 3.71-3.62 (m, |
| | | | | 2H), 2.44 (s, 3H), 2.29-2.19 (m, |
| | | | | 1H), 2.17-2.04 (m, 3H), 2.04- |
| | | | | 1.98 (m, 1H), 1.95-1.84 (m, 1H), |
| | | | | 1.51-1.45 (m, 4H), 1.28-1.24 (m, |
| | | | | 4H), 0.92 (s, 9H). |
| 46 | A | (2S,4R)-4-hydroxy-1-((S)-2- | 891.4 | 1H NMR (400 MHz, DMSO-d6) δ |
| | | (12-(3-(3-(2-hydroxyphenyl)- | | 13.89 (s, 1H), 12.54 (s, 1H), 8.98 (s, |
| | | 7H-pyrrolo[2,3-c]pyridazin-5- | | 1H), 8.60 (s, 1H), 8.56 (t, J = 6.1 |
| | | yl)azetidin-1-yl)-12- | | Hz, 1H), 8.12 (s, 1H), 8.05 (d, J = |
| | | oxododecanamido)-3,3- | | 8.1 Hz, 1H), 7.83 (d, J = 9.4 Hz, |
| | | dimethylbutanoyl)-N-(4-(4- | | 1H), 7.40 (q, J = 8.2 Hz, 4H), 7.30 |
| | | methylthiazol-5- | | (t, J = 7.6 Hz, 1H), 7.01-6.92 (m, |
| | | yl)benzyl)pyrrolidine-2- | | 2H), 5.12 (d, J = 3.5 Hz, 1H), 4.60 |
| | | carboxamide | | (t, J = 8.4 Hz, 1H), 4.54 (d, J = 9.4 |
| | | | | Hz, 1H), 4.48-4.38 (m, 2H), 4.37- |
| | | | | 4.29 (m, 2H), 4.29-4.16 (m, 2H), |
| | | | | 4.15-4.06 (m, 1H), 4.06-3.98 (m, |
| | | | | 1H), 3.68-3.62 (m, 2H), 2.44 (s, |
| | | | | 3H), 2.30-2.18 (m, 1H), 2.14- |
| | | | | 2.07 (m, 3H), 2.06-1.99 (m, 1H), |
| | | | | 1.97-1.83 (m, 1H), 1.54-1.46 (m, |
| | | | | 4H), 1.30-1.20 (m, 12H), 0.93 (s, |
| | | | | 9H). |
| 47 | A | (2S,4R)-4-hydroxy-1-((S)-2- | 806.9 | 1H NMR (400 MHz, DMSO-d6) δ |
| | | (6-(3-(3-(2-hydroxyphenyl)- | | 13.09 (s, 1H), 8.98 (s, 1H), 8.66 (s, |
| | | 7H-pyrrolo[2,3-c]pyridazin-5- | | 1H), 8.59-8.54 (m, 1H), 8.39 (s, |
| | | yl)azetidin-1-yl)-6- | | 1H), 7.91-7.84 (m, 2H), 7.45- |
| | | oxohexanamido)-3,3- | | 7.33 (m, 5H), 7.08-6.97 (m, 2H), |
| | | dimethylbutanoyl)-N-(4-(4- | | 4.61 (t, 1H), 4.54 (d, J = 9.3 Hz, |
| | | methylthiazol-5- | | 1H), 4.48-4.37 (m, 2H), 4.37- |
| | | yl)benzyl)pyrrolidine-2- | | 4.30 (m, 2H), 4.30-4.21 (m, 2H), |
| | | carboxamide | | 4.21-4.12 (m, 1H), 4.07-3.99 (m, |
| | | | | 1H), 3.71-3.59 (m, 2H), 2.44 (s, |
| | | | | 3H), 2.32-2.24 (m, 1H), 2.19- |
| | | | | 2.09 (m, 3H), 2.07-1.98 (m, 1H), |
| | | | | 1.95-1.84 (m, 1H), 1.53-1.47 (m, |
| | | | | 4H), 0.92 (s, 9H). |
| 48 | B | (2S,4R)-4-hydroxy-1-((R)-2- | 874.1 | 1H NMR (400 MHz, Methanol-d4) δ |
| | | (3-(2-(4-(3-(3-(2- | | 8.78 (d, J = 4.7 Hz, 1H), 8.35 (d, J = |
| | | hydroxyphenyl)-7H- | | 3.3 Hz, 1H), 7.88-7.81 (m, 1H), |
| | | pyrrolo[2,3-c]pyridazin-6- | | 7.39-7.26 (m, 4H), 7.19 (td, J = |
| | | yl)azetidin-1-yl)piperidin-1- | | 7.6, 1.5 Hz, 1H), 6.88 (dd, J = 8.0, |
| | | yl)ethoxy)isoxazol-5-yl)-3- | | 6.7 Hz, 2H), 6.48 (d, J = 4.0 Hz, |
| | | methylbutanoyl)-N-((S)-1-(4- | | 1H), 5.92 (s, 1H), 4.94 (d, J = 7.1 |
| | | (4-methylthiazol-5- | | Hz, 1H), 4.42 (t, J = 8.2 Hz, 1H), |
| | | yl)phenyl)ethyl)pyrrolidine-2- | | 4.32 (d, J = 16.7 Hz, 1H), 4.25 (t, J = |
| | | carboxamide | | 5.5 Hz, 2H), 3.86 (p, J = 7.8 Hz, |
| | | | | 1H), 3.74 (t, J = 7.5 Hz, 3H), 3.69- |
| | | | | 3.43 (m, 2H), 3.34 (t, J = 7.6 Hz, |
| | | | | 2H), 2.92 (d, J = 11.2 Hz, 2H), 2.73 |
| | | | | (q, J = 6.0, 5.4 Hz, 2H), 2.38 (d, J = |
| | | | | 3.0 Hz, 3H), 2.34-2.03 (m, 5H), |
| | | | | 1.92-1.78 (m, 1H), 1.74 (d, J = |
| | | | | 12.7 Hz, 2H), 1.43 (d, J = 7.0 Hz, |
| | | | | 3H), 1.31 (q, J = 11.3 Hz, 2H), 0.96 |
| | | | | (d, J = 6.5 Hz, 3H), 0.81 (d, J = |
| | | | | 10.2, 6.7 Hz, 3H). |
| 49 | B | (2S,4R)-4-hydroxy-1-((2R)- | 861 | 1H NMR (400 MHz, DMSO-d6) δ |
| | | 2-(3-(2-(3-(3-(3-(2- | | 14.16 (d, J = 2.5 Hz, 1H), 12.56 (s, |
| | | hydroxyphenyl)-7H- | | 1H), 8.99 (s, 1H), 8.55 (d, J = 3.5 |
| | | pyrrolo[2,3-c]pyridazin-6- | | Hz, 1H), 8.42 (d, J = 7.7 Hz, 1H), |
| | | yl)azetidin-1-yl)pyrrolidin-1- | | 8.07-8.00 (m, 1H), 7.49-7.40 (m, |
| | | yl)ethoxy)isoxazol-5-yl)-3- | | 2H), 7.40-7.33 (m, 2H), 7.33- |
| | | methylbutanoyl)-N-((S)-1-(4- | | 7.25 (m, 1H), 6.95 (t, J = 8.0 Hz, |
| | | (4-methylthiazol-5- | | 2H), 6.55 (d, J = 2.5 Hz, 1H), 6.10 |
| | | yl)phenyl)ethyl)pyrrolidine-2- | | (s, 1H), 5.11 (d, J = 3.1 Hz, 1H), |
| | | carboxamide | | 4.90 (q, J = 7.3 Hz, 1H), 4.37 (t, J = |
| | | | | 7.9 Hz, 1H), 4.28 (s, 1H), 4.21 (t, J = |
| | | | | 5.7 Hz, 2H), 3.81 (q, J = 7.4 Hz, |
| | | | | 1H), 3.74-3.56 (m, 4H), 3.44 (d, J = |
| | | | | 10.7 Hz, 1H), 3.26 (d, J = 7.3 Hz, |
| | | | | 3H), 3.02 (s, 1H), 2.76 (s, 2H), 2.45 |
| | | | | (s, 3H), 2.31-2.14 (m, 3H), 2.03 (t, |
| | | | | J = 10.1 Hz, 1H), 1.83-1.73 (m, |
| | | | | 2H), 1.51-1.42 (m, 2H), 1.37 (d, J = |
| | | | | 7.0 Hz, 3H), 0.96 (t, J = 6.4 Hz, |
| | | | | 3H), 0.81 (dd, J = 14.5, 6.7 Hz, 3H). |
| 50 | A | (2S,4R)-4-hydroxy-1-((S)-2- | 751.35 | 1H NMR (300 MHz, DMSO-d6) δ |
| | | ((3-(3-(3-(2-hydroxyphenyl)- | | 14.07 (s, 1H), 12.73 (s, 1H), 8.98 (s, |
| | | 7H-pyrrolo[2,3-c]pyridazin-6- | | 1H), 8.58 (dd, J = 5.5, 2.5 Hz, 2H), |
| | | yl)azetidin-1-yl)-3- | | 8.14-7.94 (m, 1H), 7.40 (d, J = 2.4 |
| | | oxopropyl)amino)-3,3- | | Hz, 4H), 7.34-7.24 (m, 1H), 6.96 |
| | | dimethylbutanoyl)-N-(4-(4- | | (t, J = 8.0 Hz, 2H), 6.65 (d, J = 7.7 |
| | | methylthiazol-5- | | Hz, 1H), 5.12 (s, 1H), 4.73-4.41 |
| | | yl)benzyl)pyrrolidine-2- | | (m, 2H), 4.32 (dd, J = 27.7, 5.9 Hz, |
| | | carboxamide | | 5H), 4.11 (dd, J = 18.3, 7.8 Hz, 2H), |
| | | | | 3.77-3.44 (m, 3H), 3.05 (d, J = 3.0 |
| | | | | Hz, 1H), 2.85-2.64 (m, 2H), 2.44 |
| | | | | (s, 3H), 2.34-2.13 (m, 2H), 2.00 |
| | | | | (m, 2H), 1.06-0.74 (m, 9H). |
| 51 | B | (2S,4R)-4-hydroxy-1-((R)-2- | 819.35 | 1H NMR (400 MHz, DMSO-d6) δ |
| | | (3-(2-(4-(3-(2- | | 14.21 (s, 1H), 12.46 (s, 1H), 8.98 (s, |
| | | hydroxyphenyl)-7H- | | 1H), 8.53 (d, J = 3.3 Hz, 1H), 8.42 |
| | | pyrrolo[2,3-c]pyridazin-6- | | (d, J = 7.7 Hz, 1H), 8.06-7.99 (m, |
| | | yl)piperidin-1- | | 1H), 7.49-7.41 (m, 2H), 7.37 (d, J = |
| | | yl)ethoxy)isoxazol-5-yl)-3- | | 8.2 Hz, 2H), 7.28 (td, J = 7.7, 7.0, |
| | | methylbutanoyl)-N-((S)-1-(4- | | 1.5 Hz, 1H), 6.95 (t, J = 8.8 Hz, 2H), |
| | | (4-methylthiazol-5- | | 6.43 (d, J = 2.6 Hz, 1H), 6.12 (s, |
| | | yl)phenyl)ethyl)pyrrolidine-2- | | 1H), 5.11 (d, J = 3.6 Hz, 1H), 4.91 |
| | | carboxamide | | (t, J = 7.2 Hz, 1H), 4.37 (t, J = 7.9 |
| | | | | Hz, 1H), 4.28 (t, J = 5.3 Hz, 3H), |
| | | | | 3.75-3.62 (m, 2H), 3.45-3.40 (m, |
| | | | | 1H), 3.05 (d, J = 11.1 Hz, 2H), 2.88- |
| | | | | 2.83 (m, 1H), 2.74 (t, J = 5.7 Hz, |
| | | | | 2H), 2.45 (s, 3H), 2.19 (t, J = 11.4 |
| | | | | Hz, 3H), 2.03 (d, J = 12.3 Hz, 3H), |
| | | | | 1.83 (d, J = 10.6 Hz, 3H), 1.38 (d, |
| | | | | J = 7.0 Hz, 3H), 0.96 (d, J = 6.4 Hz, |
| | | | | 3H), 0.82 (dd, J = 14.1, 6.7 Hz, 3H). |
| 52 | B | (2S,4R)-4-hydroxy-1-((2R)- | 805.1 | 1H NMR (400 MHz, DMSO-d6) δ |
| | | 2-(3-(2-(3-(3-(2- | | 14.21 (s, 1H), 12.50 (s, 1H), 8.99 (s, |
| | | hydroxyphenyl)-7H- | | 1H), 8.53 (d, J = 3.5 Hz, 1H), 8.42 |
| | | pyrrolo[2,3-c]pyridazin-6- | | (d, J = 7.7 Hz, 1H), 8.06-7.98 (m, |
| | | yl)pyrrolidin-1- | | 1H), 7.48-7.40 (m, 2H), 7.36 (d, J = |
| | | yl)ethoxy)isoxazol-5-yl)-3- | | 8.3 Hz, 2H), 7.29 (ddd, J = 8.5, |
| | | methylbutanoyl)-N-((S)-1-(4- | | 7.2, 1.6 Hz, 1H), 7.00-6.89 (m, |
| | | (4-methylthiazol-5- | | 2H), 6.49 (s, 1H), 6.12 (s, 1H), 5.11 |
| | | yl)phenyl)ethyl)pyrrolidine-2- | | (d, J = 3.6 Hz, 1H), 4.91 (p, J = 7.5 |
| | | carboxamide | | Hz, 1H), 4.37 (t, J = 7.9 Hz, 1H), |
| | | | | 4.28 (d, J = 5.8 Hz, 3H), 3.74-3.53 |
| | | | | (m, 3H), 3.53-3.42 (m, 1H), 3.14 |
| | | | | (s, 1H), 2.90-2.67 (m, 5H), 2.45 |
| | | | | (d, J = 3.2 Hz, 3H), 2.30-2.15 (m, |
| | | | | 2H), 2.05-1.98 (m, 2H), 1.83- |
| | | | | 1.72 (m, 1H), 1.37 (d, J = 7.0 Hz, |
| | | | | 3H), 0.96 (d, J = 6.4 Hz, 3H), 0.79 |
| | | | | (d, J = 6.7 Hz, 3H). |
| 53 | B | (2S,4R)-4-hydroxy-1-((R)-2- | 817.5 | 1H NMR (400 MHz, DMSO-d6) δ |
| | | (3-(2-((3-(3-(2- | | 14.16 (s, 1H), 12.61 (d, J = 1.9 Hz, |
| | | hydroxyphenyl)-7H- | | 1H), 8.98 (d, J = 2.4 Hz, 1H), 8.54 |
| | | pyrrolo[2,3-c]pyridazin-6- | | (d, J = 3.7 Hz, 1H), 8.43 (d, J = 7.6 |
| | | yl)bicyclo[1.1.1]pentan-1- | | Hz, 1H), 8.02 (dt, J = 8.2, 2.2 Hz, |
| | | yl)amino)ethoxy)isoxazol-5- | | 1H), 7.48-7.41 (m, 2H), 7.37 (d, J = |
| | | yl)-3-methylbutanoyl)-N-((S)- | | 8.2 Hz, 2H), 7.33-7.24 (m, 1H), |
| | | 1-(4-(4-methylthiazol-5- | | 7.00-6.91 (m, 2H), 6.38 (d, J = 1.8 |
| | | yl)phenyl)ethyl)pyrrolidine-2- | | Hz, 1H), 6.10 (s, 1H), 5.12 (d, J = |
| | | carboxamide | | 3.7 Hz, 1H), 4.91 (p, J = 7.4 Hz, |
| | | | | 1H), 4.38 (t, J = 7.9 Hz, 1H), 4.29 |
| | | | | (s, 1H), 4.19 (t, J = 5.7 Hz, 2H), |
| | | | | 3.75-3.63 (m, 2H), 3.53-3.42 (m, |
| | | | | 1H), 2.93 (t, J = 5.8 Hz, 2H), 2.45 |
| | | | | (d, J = 3.3 Hz, 3H), 2.29-2.21 (m, |
| | | | | 1H), 2.19 (s, 6H), 2.03 (t, J = 9.6 |
| | | | | Hz, 1H), 1.84-1.73 (m, 1H), 1.38 |
| | | | | (d, J = 7.0 Hz, 3H), 0.96 (d, J = 6.4 |
| | | | | Hz, 3H), 0.81 (d, J = 6.6 Hz, 3H). |
| 54 | B | (2S,4R)-4-hydroxy-1-((R)-2- | 831.6 | 1H NMR (400 MHz, DMSO-d6) δ |
| | | (3-(2-(6-(3-(2- | | 14.21 (s, 1H), 12.51 (s, 1H), 8.99 (s, |
| | | hydroxyphenyl)-7H- | | 1H), 8.52 (d, J = 2.9 Hz, 1H), 8.42 |
| | | pyrrolo[2,3-c]pyridazin-6-yl)- | | (d, J = 7.6 Hz, 1H), 8.03 (dd, J = |
| | | 2-azaspiro[3.3]heptan-2- | | 8.1, 1.7 Hz, 1H), 7.50-7.41 (m, |
| | | yl)ethoxy)isoxazol-5-yl)-3- | | 2H), 7.41-7.33 (m, 2H), 7.28 (td, J = |
| | | methylbutanoyl)-N-((S)-1-(4- | | 7.7, 7.1, 1.5 Hz, 1H), 6.99-6.90 |
| | | (4-methylthiazol-5- | | (m, 2H), 6.45 (d, J = 1.9 Hz, 1H), |
| | | yl)phenyl)ethyl)pyrrolidine-2- | | 6.10 (s, 1H), 5.12 (d, J = 3.6 Hz, |
| | | carboxamide | | 1H), 4.91 (p, J = 7.4 Hz, 1H), 4.37 |
| | | | | (t, J = 7.9 Hz, 1H), 4.29 (s, 1H), |
| | | | | 4.15 (s, 2H), 3.74-3.41 (m, 8H), |
| | | | | 2.87 (s, 3H), 2.48-2.43 (m, 5H), |
| | | | | 2.29-2.14 (m, 2H), 2.03 (dd, J = |
| | | | | 12.3, 8.4 Hz, 1H), 1.84-1.73 (m, |
| | | | | 1H), 1.38 (d, J = 7.0 Hz, 3H), 0.96 |
| | | | | (d, J = 6.4 Hz, 3H), 0.80 (d, J = 6.6 |
| | | | | Hz, 3H). |
| 55 | A | (2S,4R)-4-hydroxy-1-((S)-2- | 864.25 | 1H NMR (400 MHz, DMSO-d6) δ |
| | | (6-((3-(3-(3-(2- | | 14.21-13.86 (m, 1H), 8.98 (s, 1H), |
| | | hydroxyphenyl)-7H- | | 8.58 (s, 1H), 8.54 (t, J = 6.0 Hz, |
| | | pyrrolo[2,3-c]pyridazin-6- | | 1H), 8.30 (s, 1H), 8.07-8.00 (m, |
| | | yl)azetidin-1-yl)-3- | | 1H), 7.83 (d, J = 9.3 Hz, 1H), 7.40 |
| | | oxopropyl)amino)hexanamido)- | | (q, J = 8.3 Hz, 4H), 7.33-7.25 (m, |
| | | 3,3-dimethylbutanoyl)-N- | | 1H), 7.00-6.91 (m, 2H), 6.66 (s, |
| | | (4-(4-methylthiazol-5- | | 1H), 4.61-4.51 (m, 2H), 4.48- |
| | | yl)benzyl)pyrrolidine-2- | | 4.38 (m, 2H), 4.35 (s, 2H), 4.29 (t, |
| | | carboxamide | | J = 8.8 Hz, 2H), 4.26-4.12 (m, 2H), |
| | | | | 4.12-4.03 (m, 1H), 3.65 (s, 1H), |
| | | | | 2.83-2.75 (m, 2H), 2.60-2.56 (m, |
| | | | | 2H), 2.44 (s, 3H), 2.33-2.27 (m, |
| | | | | 3H), 2.16-2.07 (m, 1H), 2.05- |
| | | | | 1.98 (m, 1H), 1.96-1.88 (m, 1H), |
| | | | | 1.54-1.38 (m, 4H), 1.30-1.22 (m, |
| | | | | 2H), 0.93 (s, 9H). |
| 56 | A | (2S,4R)-4-hydroxy-1-((S)-2- | 892.1 | 1H NMR (400 MHz, DMSO-d6) δ |
| | | (8-((3-(3-(3-(2- | | 8.98 (s, 1H), 8.63 (s, 1H), 8.57 (t, |
| | | hydroxyphenyl)-7H- | | J = 6.1 Hz, 1H), 8.35 (s, 1H), 8.12 (s, |
| | | pyrrolo[2,3-c]pyridazin-5- | | 1H), 8.08-8.04 (m, 1H), 7.84 (d, |
| | | yl)azetidin-1-yl)-3- | | J = 9.4 Hz, 1H), 7.40 (q, J = 8.2 Hz, |
| | | oxopropyl)amino)octanamido)- | | 4H), 7.30 (td, J = 7.7, 7.0, 1.6 Hz, |
| | | 3,3-dimethylbutanoyl)-N- | | 1H), 6.97 (t, J = 7.6 Hz, 2H), 4.61 |
| | | (4-(4-methylthiazol-5- | | (d, J = 8.5 Hz, 1H), 4.54 (d, J = 9.4 |
| | | yl)benzyl)pyrrolidine-2- | | Hz, 1H), 4.43 (q, J = 7.3 Hz, 2H), |
| | | carboxamide | | 4.37-4.32 (m, 2H), 4.28 (t, J = 7.2 |
| | | | | Hz, 1H), 4.21 (dd, J = 15.9, 5.4 Hz, |
| | | | | 1H), 4.14 (t, J = 7.7 Hz, 1H), 4.05- |
| | | | | 4.01 (m, 1H), 3.67-3.63 (m, 2H), |
| | | | | 2.81 (d, J = 7.4 Hz, 2H), 2.60-2.55 |
| | | | | (m, 3H), 2.44 (s, 3H), 2.36-2.34 |
| | | | | (m, 1H), 2.27-2.21 (m, 1H), 2.14- |
| | | | | 2.05 (m, 2H), 2.01 (d, J = 2.2 Hz, |
| | | | | 1H), 1.94-1.86 (m, 1H), 1.53- |
| | | | | 1.34 (m, 5H), 1.23 (s, 6H), 0.93 (s, |
| | | | | 9H). |
| 57 | A | (2S,4R)-4-hydroxy-1-((S)-2- | 864.50 | 1H NMR (400 MHz, DMSO-d6) δ |
| | | (6-((3-(3-(3-(2- | | 8.98 (s, 1H), 8.62 (s, 1H), 8.56 (t, |
| | | hydroxyphenyl)-7H- | | J = 6.1 Hz, 1H), 8.12 (s, 1H), 8.08- |
| | | pyrrolo[2,3-c]pyridazin-5- | | 8.04 (m, 1H), 7.84 (d, J = 9.3 Hz, |
| | | yl)azetidin-1-yl)-3- | | 1H), 7.40 (q, J = 8.2 Hz, 4H), 7.30 |
| | | oxopropyl)amino)hexanamido)- | | (td, J = 7.6, 1.5 Hz, 1H), 7.02-6.94 |
| | | 3,3-dimethylbutanoyl)-N- | | (m, 2H), 4.57 (dd, J = 27.0, 8.8 Hz, |
| | | (4-(4-methylthiazol-5- | | 2H), 4.47-4.39 (m, 2H), 4.37- |
| | | yl)benzyl)pyrrolidine-2- | | 4.31 (m, 2H), 4.28-4.12 (m, 3H), |
| | | carboxamide | | 4.03 (dd, J = 9.2, 6.1 Hz, 1H), 3.69- |
| | | | | 3.61 (m, 2H), 2.74 (t, J = 6.8 Hz, |
| | | | | 2H), 2.56-2.53 (m, 2H), 2.44 (s, |
| | | | | 3H), 2.30-2.21 (m, 3H), 2.13- |
| | | | | 2.01 (m, 2H), 1.93-1.86 (m, 1H), |
| | | | | 1.50-1.34 (m, 4H), 1.23 (t, J = 7.6 |
| | | | | Hz, 2H), 0.93 (s, 9H). |
| 58 | B | (2S,4R)-4-hydroxy-1-((R)-2- | 903.85 | 1H NMR (300 MHz, DMSO-d6) δ |
| | | (3-(2-(4-(2-(3-(3-(2- | | 14.19 (s, 1H), 8.99 (s, 1H), 8.56 (s, |
| | | hydroxyphenyl)-7H- | | 1H), 8.42 (d, J = 7.6 Hz, 1H), 8.04 |
| | | pyrrolo[2,3-c]pyridazin-6- | | (d, J = 7.8 Hz, 1H), 7.54-7.20 (m, |
| | | yl)azetidin-1- | | 6H), 6.96 (t, J = 7.7 Hz, 2H), 6.55 |
| | | yl)ethyl)piperazin-1- | | (s, 1H), 6.10 (s, 1H), 5.10 (s, 1H), |
| | | yl)ethoxy)isoxazol-5-yl)-3- | | 4.91 (t, J = 7.2 Hz, 1H), 4.43-4.32 |
| | | methylbutanoyl)-N-((S)-1-(4- | | (m, 1H), 4.32-4.18 (m, 3H), 3.86 |
| | | (4-methylthiazol-5- | | (t, J = 7.4 Hz, 1H), 3.79-3.44 (m, |
| | | yl)phenyl)ethyl)pyrrolidine-2- | | 8H), 3.40 (d, J = 17.5 Hz, 2H), 2.73 |
| | | carboxamide | | (p, J = 1.9 Hz, 4H), 2.70-2.53 (m, |
| | | | | 10H), 2.02 (d, J = 9.2 Hz, 1H), 1.86- |
| | | | | 1.75 (m, 1H), 1.42 (m, J = 22.0, |
| | | | | 7.0 Hz, 4H), 0.96 (d, J = 6.4 Hz, |
| | | | | 3H), 0.80 (d, J = 6.7 Hz, 3H). |
| 59 | B | (2S,4R)-4-hydroxy-1-((R)-2- | 848.25 | 1H NMR (400 MHz, DMSO-d6) δ |
| | | (3-(2-((2-(3-(3-(2- | | 14.17 (s, 1H), 12.52 (s, 1H), 8.98 (s, |
| | | hydroxyphenyl)-7H- | | 1H), 8.55 (d, J = 5.8 Hz, 1H), 8.41 |
| | | pyrrolo[2,3-c]pyridazin-6- | | (d, J = 7.7 Hz, 1H), 8.07-8.00 (m, |
| | | yl)azetidin-1- | | 1H), 7.47-7.40 (m, 2H), 7.36 (d, |
| | | yl)ethyl)(methyl)amino)ethoxy)isoxazol- | | J = 8.2 Hz, 2H), 7.29 (ddd, J = 8.5, |
| | | 5-yl)-3- | | 7.1, 1.6 Hz, 1H), 7.00-6.91 (m, |
| | | methylbutanoyl)-N-((S)-1-(4- | | 2H), 6.55 (d, J = 4.6 Hz, 1H), 6.08 |
| | | (4-methylthiazol-5- | | (s, 1H), 5.10 (s, 1H), 4.94-4.86 |
| | | yl)phenyl)ethyl)pyrrolidine-2- | | (m, 1H), 4.37 (t, J = 7.9 Hz, 1H), |
| | | carboxamide | | 4.28 (s, 1H), 4.22 (t, J = 5.7 Hz, |
| | | | | 2H), 3.90-3.81 (m, 1H), 3.67 (td, |
| | | | | J = 14.5, 13.9, 6.9 Hz, 4H), 3.44 (d, |
| | | | | J = 11.0 Hz, 1H), 3.30 (s, 2H), 2.73 (t, |
| | | | | J = 5.8 Hz, 2H), 2.58 (s, 2H), 2.45 |
| | | | | (d, J = 3.5 Hz, 3H), 2.40 (t, J = 6.9 |
| | | | | Hz, 2H), 2.25 (s, 4H), 2.02 (t, J = |
| | | | | 10.1 Hz, 1H), 1.83-1.72 (m, 1H), |
| | | | | 1.36 (d, J = 7.0 Hz, 3H), 0.94 (d, J = |
| | | | | 6.4 Hz, 3H), 0.79 (dd, J = 14.8, 6.7 |
| | | | | Hz, 3H). |
| 60 | A | (2S,4R)-4-hydroxy-1-((S)-2- | 765.45 | 1H NMR (400 MHz, DMSO-d6) δ |
| | | (4-(3-(3-(2-hydroxyphenyl)- | | 14.14 (s, 1H), 12.49 (s, 1H), 8.97 |
| | | 7H-pyrrolo[2,3-c]pyridazin-6- | | (s, 1H), 8.54 (s, 2H), 8.02 (dd, J = |
| | | yl)azetidin-1-yl)butanamido)- | | 8.0, 1.6 Hz, 1H), 7.89 (d, J = 9.4 |
| | | 3,3-dimethylbutanoyl)-N-(4- | | Hz, 1H), 7.40 (q, J = 8.3 Hz, 4H), |
| | | (4-methylthiazol-5- | | 7.29 (ddd, J = 8.4, 7.1, 1.6 Hz, 1H), |
| | | yl)benzyl)pyrrolidine-2- | | 7.00-6.91 (m, 2H), 6.55 (s, 1H), |
| | | carboxamide | | 5.13 (s, 1H), 4.55 (d, J = 9.4 Hz, |
| | | | | 1H), 4.48-4.39 (m, 2H), 4.36 (S, |
| | | | | 1H), 4.22 (dd, J = 15.8, 5.4 Hz, 1H), |
| | | | | 3.85 (t, J = 7.3 Hz, 1H), 3.65 (q, J = |
| | | | | 6.6, 6.0 Hz, 4H), 3.31-3.20 (m, 2H), |
| | | | | 2.44 (m, 5H), 2.27 (m,1H), 2.18 m, |
| | | | | 1H), 2.04 (t, J = 10.2 Hz, 1H), 1.91 |
| | | | | (m, 1H), 1.54 (dt, J = 14.4, 7.0 Hz, |
| | | | | 2H), 0.95 (s, 9H). |
| 61 | A | (2S,4R)-4-hydroxy-1-((S)-2- | 937.40 | 1H NMR (400 MHz, DMSO-d6) δ |
| | | (2-(3-(3-(2-hydroxyphenyl)- | | 8.96 (s, 1H), 8.64 (t, J = 6.1 Hz, |
| | | 7H-pyrrolo[2,3-c]pyridazin-6- | | 1H), 8.55 (s, 1H), 7.99 (d, J = 7.5 |
| | | yl)azetidin-1-yl)acetamido)- | | Hz, 1H), 7.48-7.35 (m, 4H), 7.33- |
| | | 3,3-dimethylbutanoyl)-N-(4- | | 7.25 (m, 1H), 7.03-6.90 (m, 2H), |
| | | (4-methylthiazol-5- | | 6.66 (s, 1H), 4.54 (s, 1H), 4.50- |
| | | yl)benzyl)pyrrolidine-2- | | 4.32 (m, 3H), 4.27 (d, J = 5.5 Hz, |
| | | carboxamide | | 1H), 4.25-4.03 (m, 3H), 3.68 (dd, |
| | | | | J = 10.7, 3.9 Hz, 3H), 3.60 (d, J = |
| | | | | 11.2 Hz, 3H), 2.43 (s, 3H), 2.07 (t, |
| | | | | J = 10.4 Hz, 1H), 1.90 (ddd, J = 13.0, |
| | | | | 8.8, 4.6 Hz, 1H), 0.96 (s, 9H). |
| 62 | B | (2S,4R)-4-hydroxy-1-((S)-2- | 791.5 | 1H NMR (300 MHz, DMSO-d6) δ |
| | | (3-(2-(3-(3-(2- | | 14.17 (s, 1H), 12.55 (s, 1H), 9.03- |
| | | hydroxyphenyl)-7H- | | 8.88 (m, 1H), 8.56 (d, J = 4.1 Hz, |
| | | pyrrolo[2,3-c]pyridazin-6- | | 1H), 8.33 (d, J = 7.8 Hz, 1H), 8.03 |
| | | yl)azetidin-1- | | (d, J = 7.9 Hz, 1H), 7.52-7.25 (m, |
| | | yl)ethoxy)isoxazol-5-yl)-3- | | 5H), 7.01-6.89 (m, 2H), 6.56 (d, |
| | | methylbutanoyl)-N-((S)-1-(4- | | J = 5.5 Hz, 1H), 6.12 (d, J = 16.3 Hz, |
| | | (4-methylthiazol-5- | | 1H), 5.11 (d, J = 3.6 Hz, 1H), 4.88 |
| | | yl)phenyl)ethyl)pyrrolidine-2- | | (t, J = 7.3 Hz, 1H), 4.43 (t, J = 7.7 |
| | | carboxamide | | Hz, 1H), 4.27 (s, 1H), 4.14 (t, J = |
| | | | | 5.5 Hz, 2H), 3.88 (p, J = 7.1 Hz, |
| | | | | 1H), 3.73 (q, J = 8.6, 7.4 Hz, 3H), |
| | | | | 3.61-3.46 (m, 2H), 3.40 (d, J = 7.6 |
| | | | | Hz, 2H), 2.83 (t, J = 5.4 Hz, 2H), |
| | | | | 2.47 (s, 1H), 2.45 (s, 2H), 2.05 (s, |
| | | | | 1H), 1.77 (m, J = 12.4, 6.0 Hz, 1H), |
| | | | | 1.46 (d, J = 7.0 Hz, 1H), 1.35 (d, J = |
| | | | | 7.0 Hz, 3H), 0.97 (d, J = 6.6 Hz, |
| | | | | 2H), 0.84 (d, J = 6.8 Hz, 3H), 0.76 |
| | | | | (d, J = 6.7 Hz, 1H). |
| 63 | B | (2S,4R)-1-((S)-2-(1- | 825.50 | 1H NMR (400 MHz, DMSO-d6) δ |
| | | fluorocyclopropane-1- | | 14.14 (s, 1H), 12.62 (s, 1H), 8.99 (s, |
| | | carboxamido)-3,3- | | 1H), 8.61-8.47 (m, 2H), 8.03 (dd, |
| | | dimethylbutanoyl)-4- | | J = 8.0, 1.6 Hz, 1H), 7.42 (d, J = 7.8 |
| | | hydroxy-N-(2-(2-(3-(3-(2- | | Hz, 1H), 7.29 (ddd, J = 8.5, 7.2, 1.6 |
| | | hydroxyphenyl)-7H- | | Hz, 2H), 7.02 (d, J = 1.7 Hz, 1H), |
| | | pyrrolo[2,3-c]pyridazin-6- | | 6.99-6.92 (m, 3H), 6.60 (s, 1H), |
| | | yl)azetidin-1-yl)ethoxy)-4-(4- | | 5.16 (d, J = 3.6 Hz, 1H), 4.58 (d, J = |
| | | methylthiazol-5- | | 9.2 Hz, 1H), 4.52 (t, J = 8.2 Hz, 1H), |
| | | yl)benzyl)pyrrolidine-2- | | 4.38-4.29 (m, 2H), 4.23 (dd, J = |
| | | carboxamide | | 16.7, 5.6 Hz, 1H), 4.12 (s, 2H), 3.90 |
| | | | | (d, J = 42.0 Hz, 3H), 3.67-3.46 (m, |
| | | | | 4H), 2.98 (s, 2H), 2.47 (s, 3H), 2.15- |
| | | | | 2.03 (m, 1H), 1.91 (ddd, J = 13.1, |
| | | | | 8.9, 4.4 Hz, 1H), 1.42-1.29 (m, |
| | | | | 2H), 1.23-1.14 (m, 2H), 0.93 (s, |
| | | | | 9H). |
| 64 | B | (2S,4R)-4-hydroxy-1-((R)-2- | 876.5 | 1H NMR (400 MHz, DMSO-d6) δ |
| | | (3-(2-((3-(3-(3-(2- | | 14.06 (s, 1H), 12.72 (s, 1H), 8.98 (s, |
| | | hydroxyphenyl)-7H- | | 1H), 8.59 (d, J = 5.0 Hz, 1H), 8.40 |
| | | pyrrolo[2,3-c]pyridazin-6- | | (d, J = 7.7 Hz, 1H), 8.08-8.01 (m, |
| | | yl)azetidin-1-yl)-3- | | 1H), 7.48-7.40 (m, 2H), 7.36 (d, |
| | | oxopropyl)(methyl)ami- | | J = 8.1 Hz, 2H), 7.33-7.25 (m, 1H), |
| | | no)ethoxy)isoxazol-5-yl)-3- | | 6.96 (t, J = 8.7 Hz, 2H), 6.66 (d, J = |
| | | methylbutanoyl)-N-((S)-1-(4- | | 5.1 Hz, 1H), 6.08 (s, 1H), 5.10 (d, |
| | | (4-methylthiazol-5- | | J = 3.6 Hz, 1H), 4.90 (p, J = 7.2 Hz, |
| | | yl)phenyl)ethyl)pyrrolidine-2- | | 1H), 4.58 (t, J = 8.6 Hz, 1H), 4.37 |
| | | carboxamide | | (td, J = 8.1, 7.2, 3.2 Hz, 2H), 4.22 |
| | | | | (tt, J = 26.3, 8.0 Hz, 6H), 4.06 (dd, |
| | | | | J = 9.1, 6.1 Hz, 1H), 3.73-3.60 (m, |
| | | | | 2H), 3.45 (s, 1H), 2.72 (t, J = 5.7 |
| | | | | Hz, 2H), 2.69-2.63 (m, 1H), 2.45 |
| | | | | (s, 3H), 2.25 (d, J = 8.0 Hz, 6H), |
| | | | | 2.02 (t, J = 10.0 Hz, 1H), 1.78 (ddd, |
| | | | | J = 12.7, 7.8, 4.7 Hz, 1H), 1.37 (d, |
| | | | | J = 7.0 Hz, 3H), 0.95 (t, J = 6.1 Hz, |
| | | | | 3H), 0.80 (dd, J = 15.5, 6.6 Hz, 3H). |
| 65 | B | (2S,4R)-4-hydroxy-1-((R)-2- | 862.5 | 1H NMR (400 MHz, DMSO-d6) δ |
| | | (3-(2-((3-(3-(3-(2- | | 14.04 (s, 1H), 12.71 (s, 1H), 8.98 (s, |
| | | hydroxyphenyl)-7H- | | 1H), 8.59 (d, J = 2.8 Hz, 1H), 8.41 |
| | | pyrrolo[2,3-c]pyridazin-6- | | (d, J = 7.4 Hz, 1H), 8.08-8.01 (m, |
| | | yl)azetidin-1-yl)-3- | | 1H), 7.44 (d, J = 8.5 Hz, 2H), 7.36 |
| | | oxopropyl)amino)ethoxy)isoxazol- | | (d, J = 7.9 Hz, 2H), 7.32-7.26 (m, |
| | | 5-yl)-3-methylbutanoyl)- | | 1H), 6.96 (t, J = 8.6 Hz, 2H), 6.67 |
| | | N-((S)-1-(4-(4-methylthiazol- | | (d, J = 2.5 Hz, 1H), 6.10 (s, 1H), |
| | | 5-yl)phenyl)ethyl)pyrrolidine- | | 5.11 (d, J = 4.0 Hz, 1H), 4.92 (q, J = |
| | | 2-carboxamide | | 7.3 Hz, 1H), 4.58 (t, J = 8.6 Hz, 1H), |
| | | | | 4.38 (d, J = 7.4 Hz, 2H), 4.36-4.25 |
| | | | | (m, 4H), 4.17 (q, J = 7.6 Hz, 1H), |
| | | | | 4.12-4.05 (m, 1H), 3.74-3.62 (m, |
| | | | | 2H), 3.46 (s, 1H), 3.05 (s, 2H), 2.93 |
| | | | | (s, 2H), 2.45 (s, 3H), 2.37 (s, 2H), |
| | | | | 2.25 (d, J = 8.4 Hz, 1H), 2.03 (t, J = |
| | | | | 10.1 Hz, 1H), 1.78 (ddd, J = 12.8, |
| | | | | 7.8, 4.4 Hz, 1H), 1.37 (d, J = 6.9 |
| | | | | Hz, 3H), 0.96 (d, J = 6.4 Hz, 3H), |
| | | | | 0.79 (d, J = 6.6 Hz, 3H). |
| 66 | A | (2S,4R)-4-hydroxy-1-((S)-2- | 892.30 | 1H NMR (400 MHz, DMSO-d6) δ |
| | | (8-((3-(3-(3-(2- | | 14.03 (s, 1H), 8.98 (s, 1H), 8.58 (s, |
| | | hydroxyphenyl)-7H- | | 1H), 8.54 (t, 1H), 8.31-8.26 (m, |
| | | pyrrolo[2,3-c]pyridazin-6- | | 1H), 8.07-8.00 (m, 1H), 7.81 (d, |
| | | yl)azetidin-1-yl)-3- | | J = 9.4 Hz, 1H), 7.45-7.34 (m, 4H), |
| | | oxopropyl)amino)octanamido)- | | 7.33-7.25 (m, 1H), 7.00-6.91 (m, |
| | | 3,3-dimethylbutanoyl)-N- | | 2H), 6.66 (s, 1H), 4.61-4.51 (m, |
| | | (4-(4-methylthiazol-5- | | 2H), 4.48-4.38 (m, 2H), 4.37- |
| | | yl)benzyl)pyrrolidine-2- | | 4.33 (m, 2H), 4.34-4.25 (m, 2H), |
| | | carboxamide | | 4.26-4.14 (m, 2H), 4.12-4.03 (m, |
| | | | | 1H), 3.67-3.63 (m, 1H), 2.80 (s, |
| | | | | 2H), 2.60-2.55 (m, 2H), 2.44 (s, |
| | | | | 3H), 2.36-2.20 (m, 3H), 2.15- |
| | | | | 2.00 (m, 2H), 1.96-1.86 (m, 1H), |
| | | | | 1.44-1.40 (m, 4H), 1.25 (s, 6H), |
| | | | | 0.93 (s, 9H). |
| 305 | A | (2S,4R)-4-hydroxy-1-((R)-2- | 805.45 | 1H NMR (300 MHz, DMSO-d6) δ |
| | | (3-(2-(3-(3-(2- | | 12.98 (s, 1H), 8.99 (s, 1H), 8.63 (s, |
| | | hydroxyphenyl)-7H- | | 1H), 8.43 (d, J = 7.7 Hz, 1H), 7.95 |
| | | pyrrolo[2,3-c]pyridazin-6- | | (s, 1H), 7.45 (d, J = 8.3 Hz, 2H), |
| | | yl)azetidin-1-yl)-2- | | 7.37 (d, J = 8.4 Hz, 3H), 7.00 (t, J = |
| | | oxoethoxy)isoxazol-5-yl)-3- | | 8.9 Hz, 2H), 6.79 (s, 1H), 6.19 (s, |
| | | methylbutanoyl)-N-((S)-1-(4- | | 1H), 4.92 (t, J = 7.3 Hz, 1H), 4.79 |
| | | (4-methylthiazol-5- | | (s, 2H), 4.69 (t, J = 8.4 Hz, 1H), |
| | | yl)phenyl)ethyl)pyrrolidine-2- | | 4.52-4.36 (m, 3H), 4.29 (s, 2H), |
| | | carboxamide | | 4.19 (d, J = 8.0 Hz, 1H), 3.77-3.64 |
| | | | | (m, 2H), 3.53-3.44 (m, 2H), 2.46 |
| | | | | (s, 3H), 2.10-2.05 (m, 1H), 1.86- |
| | | | | 1.73 (m, 1H), 1.38 (d, J = 7.2 Hz, |
| | | | | 3H), 0.97 (d, J = 6.4 Hz, 3H), 0.80 |
| | | | | (d, J = 7.2 Hz, 3H). |
| 264 | A | (2S,4R)-4-hydroxy-1-((R)-2- | 858.50 | 1H NMR (400 MHz, DMSO-d6) δ |
| | | (3-(4-(3-(3-(2- | | 14.09 (s, 1H), 12.76 (s, 1H), 8.99 |
| | | hydroxyphenyl)-7H- | | (d, J = 7.5 Hz, 1H), 8.59 (s, 1H), |
| | | pyrrolo[2,3-c]pyridazin-6- | | 8.25 (d, J = 7.8 Hz, 1H), 8.04 (dd, |
| | | yl)azetidine-1- | | J = 8.0, 1.6 Hz, 1H), 7.51-7.24 (m, |
| | | carbonyl)piperidin-1- | | 5H), 7.00-6.91 (m, 2H), 6.67 (s, |
| | | yl)isoxazol-5-yl)-3- | | 1H), 6.17 (d, J = 4.1 Hz, 1H), 5.16- |
| | | methylbutanoyl)-N-((S)-1-(4- | | 4.94 (m, 1H), 4.94-4.83 (m, 1H), |
| | | (4-methylthiazol-5- | | 4.69-4.59 (m, 1H), 4.49-4.37 (m, |
| | | yl)phenyl)ethyl)pyrrolidine-2- | | 2H), 4.34-4.23 (m, 2H), 4.22- |
| | | carboxamide | | 4.13 (m, 1H), 4.12-4.02 (m, 1H), |
| | | | | 3.73-3.58 (m, 3H), 3.58-3.40 (m, |
| | | | | 2H), 3.31-3.25 (m, 1H), 2.92- |
| | | | | 2.72 (m, 2H), 2.45 (d, J = 7.0 Hz, |
| | | | | 3H), 2.30-2.20 (m, 1H), 2.08- |
| | | | | 1.88 (m, 1H), 1.85-1.64 (m, 3H), |
| | | | | 1.64-1.49 (m, 2H), 1.35 (d, J = 7.0 |
| | | | | Hz, 3H), 0.96 (d, J = 6.6 Hz, 3H), |
| | | | | 0.80 (d, J = 6.6 Hz, 3H). |
| 274 | A | (2S,4R)-4-hydroxy-1-((S)-2- | 858.50 | 1H NMR (400 MHz, DMSO-d6) δ |
| | | (3-(4-(3-(3-(2- | | 14.06 (s, 1H), 12.75 (s, 1H), 8.99 |
| | | hydroxyphenyl)-7H- | | (d, J = 2.5 Hz, 1H), 8.59 (s, 1H), |
| | | pyrrolo[2,3-c]pyridazin-6- | | 8.40 (d, J = 7.7 Hz, 1H), 8.04 (dd, |
| | | yl)azetidine-1- | | J = 8.1, 1.6 Hz, 1H), 7.50-7.26 (m, |
| | | carbonyl)piperidin-1- | | 5H), 7.00-6.92 (m, 2H), 6.68 (s, |
| | | yl)isoxazol-5-yl)-3- | | 1H), 6.15 (s, 1H), 5.10 (d, J = 3.7 |
| | | methylbutanoyl)-N-((S)-1-(4- | | Hz, 1H), 4.96-4.87 (m, 1H), 4.65 |
| | | (4-methylthiazol-5- | | (t, J = 8.5 Hz, 1H), 4.49-4.25 (m, |
| | | yl)phenyl)ethyl)pyrrolidine-2- | | 4H), 4.24-4.14 (m, 1H), 4.12- |
| | | carboxamide | | 4.04 (m, 1H), 3.76-3.62 (m, 3H), |
| | | | | 3.58 (d, J = 9.9 Hz, 1H), 3.47-3.40 |
| | | | | (m, 1H), 3.30-3.27 (m, 1H), 2.89- |
| | | | | 2.77 (m, 2H), 2.46 (s, 3H), 2.30- |
| | | | | 2.11 (m, 1H), 2.07-1.97 (m, 1H), |
| | | | | 1.84-1.66 (m, 3H), 1.65-1.50 (m, |
| | | | | 2H), 1.38 (d, J = 7.0 Hz, 3H), 0.96 |
| | | | | (t, J = 6.6 Hz, 3H), 0.80 (dd, J = 6.6 |
| | | | | Hz, 3H). |
| 151 | B | (2S,4R)-4-hydroxy-1-((R)-2- | 831.25 | 1H NMR (400 MHz, Methanol-d4) δ |
| | | (3-(2-(6-(3-(2- | | 8.86 (d, J = 8.1 Hz, 1H), 8.45 (d, J = |
| | | hydroxyphenyl)-7H- | | 4.2 Hz, 1H), 7.95 (dd, J = 25.8, 8.1 |
| | | pyrrolo[2,3-c]pyridazin-5-yl)- | | Hz, 1H), 7.76 (d, J = 11.2 Hz, 1H), |
| | | 1-azaspiro[3.3]heptan-1- | | 7.51-7.38 (m, 3H), 7.37-7.12 (m, |
| | | yl)ethoxy)isoxazol-5-yl)-3- | | 2H), 7.05-6.89 (m, 2H), 6.05 (d, |
| | | methylbutanoyl)-N-((S)-1-(4- | | J = 21.9 Hz, 1H), 5.05-4.99 (m, 1H), |
| | | (4-methylthiazol-5- | | 4.55 (d, J = 35.9 Hz, 3H), 4.40 (d, |
| | | yl)phenyl)ethyl)pyrrolidine-2- | | J = 29.1 Hz, 3H), 3.84 (dd, J = 10.9, |
| | | carboxamide | | 4.1 Hz, 1H), 3.70 (d, J = 9.9 Hz, |
| | | | | 1H), 3.62 (d, J = 10.0 Hz, 1H), 3.48 |
| | | | | (s, 1H), 3.17 (s, 1H), 3.13 (s, 1H), |
| | | | | 3.09 (d, J = 13.8 Hz, 3H), 2.47 (s, |
| | | | | 1H), 2.44-2.40 (m, 1H), 2.26 (s, |
| | | | | 1H), 2.22-2.13 (m, 1H), 1.95 (ddd, |
| | | | | J = 13.3, 9.0, 4.7 Hz, 1H), 1.58 (d, |
| | | | | J = 7.0 Hz, 1H), 1.51 (d, J = 7.0 Hz, |
| | | | | 2H), 1.28 (s, 1H), 1.21 (s, 3H), 1.06 |
| | | | | (s, 1H), 1.05 (s, 1H), 0.91 (dd, J = |
| | | | | 10.4, 6.6 Hz, 3H), 0.87-0.78 (m, |
| | | | | 1H). |
| 155 | A | (2S,4R)-4-hydroxy-1-((S)-2- | 857.00 | 1H NMR (300 MHz, DMSO-d6) δ |
| | | (3-(1-((S)-3-(2- | | 14.44 (s, 1H), 12.25 (s, 1H), 9.02- |
| | | hydroxyphenyl)-6,7,8,9- | | 8.88 (m, 1H), 8.56 (s, 1H), 8.36- |
| | | tetrahydro-5H- | | 8.22 (m, 1H), 8.08 (d, J = 7.9 Hz, |
| | | pyridazino[3,4-b]indole-6- | | 1H), 7.53-7.21 (m, 5H), 6.95 (d, |
| | | carbonyl)piperidin-4- | | J = 8.1 Hz, 2H), 6.39 (d, J = 5.6 Hz, |
| | | yl)isoxazol-5-yl)-3- | | 1H), 5.18-4.80 (m, 2H), 4.58- |
| | | methylbutanoyl)-N-((S)-1-(4- | | 4.36 (m, 2H), 4.28 (s, 1H), 4.10 (s, |
| | | (4-methylthiazol-5- | | 1H), 3.87 (d, J = 8.6 Hz, 1H), 3.64- |
| | | yl)phenyl)ethyl)pyrrolidine-2- | | 3.42 (m, 2H), 3.19 (d, J = 12.3 Hz, |
| | | carboxamide | | 1H), 3.10-2.64 (m, 7H), 2.47- |
| | | | | 2.40 (m, 2H), 2.38-2.34 (m, 1H), |
| | | | | 2.18-1.69 (m, 7H), 1.69-1.28 (m, |
| | | | | 5H), 1.09-0.68 (m, 6H). |
| 147 | A | (2S,4R)-4-hydroxy-1-[(2S)-2- | 857.50 | 1H NMR (300 MHz, DMSO-d6) δ |
| | | (3-{1-[(6S)-3-(2- | | 14.45 (s, 1H), 12.25 (s, 1H), 9.03- |
| | | hydroxyphenyl)- | | 8.94 (m, 1H), 8.62-8.41 (m, 1H), |
| | | 5H,6H,7H,8H,9H- | | 8.29 (m, 1H), 8.08 (d, J = 8.0 Hz, |
| | | pyridazino[3,4-b]indole-6- | | 1H), 7.53-7.23 (m, 5H), 6.96 (d, |
| | | carbonyl]piperidin-4-yl}-1,2- | | J = 8.3 Hz, 2H), 6.44-6.36 (m, 1H), |
| | | oxazol-5-yl)-3- | | 5.18-4.82 (m, 2H), 4.59-4.02 (m, |
| | | methylbutanoyl]-N-[(1S)-1- | | 3H), 3.88 (d, J = 8.6 Hz, 1H), 3.62- |
| | | [4-(4-methyl-1,3-thiazol-5- | | 3.51 (m, 3H), 3.10-2.85 (m, 8H), |
| | | yl)phenyl]ethyl]pyrrolidine-2- | | 2.49-2.40 (m, 4H), 2.35-1.74 (m, |
| | | carboxamide | | 6H), 1.58-1.31 (m, 5H), 0.98 (d, |
| | | | | J = 6.6 Hz, 3H), 0.89-0.72 (m, 3H). |
| 211 | B | (2S,4R)-4-hydroxy-1-((R)-2- | 845.50 | 1H NMR (300 MHz, DMSO-d6) δ |
| | | (3-(3-(6-(3-(2- | | 8.97 (s, 1H), 8.56 (s, 1H), 8.09 (d, |
| | | hydroxyphenyl)-7H- | | J = 7.9 Hz, 1H), 7.86 (s, 1H), 7.43 (d, |
| | | pyrrolo[2,3-c]pyridazin-5-yl)- | | J = 8.2 Hz, 2H), 7.36 (d, J = 8.3 Hz, |
| | | 2-azaspiro[3.3]heptan-2- | | 2H), 7.30 (t, J = 7.6 Hz, 1H), 7.03- |
| | | yl)propoxy)isoxazol-5-yl)-3- | | 6.93 (m, 2H), 6.07 (s, 1H), 4.90 (d, |
| | | methylbutanoyl)-N-((S)-1-(4- | | J = 6.8 Hz, 1H), 4.36 (t, J = 8.2 Hz, |
| | | (4-methylthiazol-5- | | 1H), 4.28 (s, 1H), 4.15 (t, J = 6.4 |
| | | yl)phenyl)ethyl)pyrrolidine-2- | | Hz, 2H), 3.74-3.59 (m, 3H), 3.38- |
| | | carboxamide | | 3.25 (m, 4H), 3.07 (s, 2H), 2.60 (d, |
| | | | | J = 11.1 Hz, 2H), 2.44 (d, J = 3.6 |
| | | | | Hz, 3H), 2.27 (t, J = 6.2 Hz, 2H), |
| | | | | 2.04 (s, 2H), 1.81-1.64 (m, 3H), |
| | | | | 1.41 (m, J = 22.9, 6.9 Hz, 3H), 0.95 |
| | | | | (d, J = 6.4 Hz, 3H), 0.86-0.75 (m, |
| | | | | 3H). |
| 231 | A | (2S,4R)-4-hydroxy-1-((S)-2- | 858.55 | 1H NMR (400 MHz, DMSO-d6) δ |
| | | (3-(4-(3-(3-(2- | | 13.94-13.86 (m, 1H), 12.63- |
| | | hydroxyphenyl)-7H- | | 12.41 (m, 1H), 8.99 (s, 1H), 8.60 (s, |
| | | pyrrolo[2,3-c]pyridazin-5- | | 1H), 8.40 (d, J = 7.8 Hz, 1H), 8.14 |
| | | yl)azetidine-1- | | (s, 1H), 8.05 (d, J = 7.8 Hz, 1H), |
| | | carbonyl)piperidin-1- | | 7.49-7.26 (m, 5H), 7.02-6.94 (m, |
| | | yl)isoxazol-5-yl)-3- | | 2H), 6.15 (s, 1H), 5.10 (d, J = 3.7 |
| | | methylbutanoyl)-N-((S)-1-(4- | | Hz, 1H), 4.97-4.87 (m, 1H), 4.76- |
| | | (4-methylthiazol-5- | | 4.64 (m, 1H), 4.41-4.25 (m, 4H), |
| | | yl)phenyl)ethyl)pyrrolidine-2- | | 4.20-4.10 (m, 1H), 4.08-4.00 (m, |
| | | carboxamide | | 1H), 3.76-3.62 (m, 3H), 3.57 (d, |
| | | | | J = 9.9 Hz, 1H), 3.47-3.40 (m, 1H), |
| | | | | 2.89-2.75 (m, 3H), 2.46 (s, 3H), |
| | | | | 2.28-2.10 (m, 1H), 2.08-1.96 (m, |
| | | | | 1H), 1.84-1.67 (m, 3H), 1.67- |
| | | | | 1.52 (m, 2H), 1.38 (d, J = 7.0 Hz, |
| | | | | 3H), 0.95 (d, J = 6.7 Hz, 3H), 0.82 |
| | | | | (d, J = 6.7 Hz, 3H). |
| 262 | B | (2S,4R)-4-hydroxy-1-((R)-2- | 805.40 | 1H NMR (300 MHz, Methanol-d4) δ |
| | | (3-(2-((S)-3-(3-(2- | | 8.92-8.84 (m, 1H), 8.72-8.66 (m, |
| | | hydroxyphenyl)-7H- | | 1H), 8.50 (s, 1H), 8.07-7.99 (m, |
| | | pyrrolo[2,3-c]pyridazin-5- | | 1H), 7.82-7.76 (m, 1H), 7.51- |
| | | yl)pyrrolidin-1- | | 7.32 (m, 4H), 7.35-7.27 (m, 1H), |
| | | yl)ethoxy)isoxazol-5-yl)-3- | | 7.06-6.97 (m, 2H), 6.05-5.97 (m, |
| | | methylbutanoyl)-N-((S)-1-(4- | | 1H), 5.26-4.95 (m, 1H), 4.61 (s, |
| | | (4-methylthiazol-5- | | 3H), 4.56-4.37 (m, 4H), 3.90- |
| | | yl)phenyl)ethyl)pyrrolidine-2- | | 3.81 (m, 4H), 3.73-3.63 (m, 6H), |
| | | carboxamide | | 3.63-3.57 (m, 2H), 3.53-3.38 (m, |
| | | | | 3H), 3.27-3.22 (m, 1H), 2.53- |
| | | | | 2.44 (m, 5H), 2.43-2.25 (m, 2H), |
| | | | | 2.21-2.15 (m, 3H), 2.12-1.87 (m, |
| | | | | 2H), 1.62-1.50 (m, 4H), 1.34- |
| | | | | 1.28 (m, 1H), 1.18-1.12 (m, 1H), |
| | | | | 1.10-1.02 (m, 3H), 0.95-0.84 (m, |
| | | | | 4H). |
| 282 | B | (2S,4R)-4-hydroxy-1-((R)-2- | 819.45 | 1H NMR (400 MHz, DMSO-d6) δ |
| | | (3-(2-(4-(3-(2- | | 14.10 (s, 1H), 12.38 (s, 1H), 8.99 (s, |
| | | hydroxyphenyl)-7H- | | 1H), 8.74 (s, 1H), 8.43 (d, J = 7.6 |
| | | pyrrolo[2,3-c]pyridazin-5- | | Hz, 1H), 8.29-8.14 (m, 1H; FA |
| | | yl)piperidin-1- | | salt, 1H), 7.84 (s, 1H), 7.49-7.25 |
| | | yl)ethoxy)isoxazol-5-yl)-3- | | (m, 5H), 6.97 (d, J = 7.7 Hz, 2H), |
| | | methylbutanoyl)-N-((S)-1-(4- | | 6.12 (s, 1H), 5.22-4.87 (m, 2H), |
| | | (4-methylthiazol-5- | | 4.43-4.26 (m, 4H), 3.75-3.62 (m, |
| | | yl)phenyl)ethyl)pyrrolidine-2- | | 2H), 3.53-3.42 (m, 1H), 3.04 (d, |
| | | carboxamide | | J = 10.9 Hz, 2H), 2.94-2.90 (m, 1H), |
| | | | | 2.71-2.64 (m, 1H), 2.46 (s, 3H), |
| | | | | 2.35-2.31 (m, 1H), 2.29-2.25 (m, |
| | | | | 1H), 2.21-2.18 (m, 1H), 2.06- |
| | | | | 1.95 (m, 4H), 1.91-1.67 (m, 3H), |
| | | | | 1.49-1.35 (m, 3H), 0.96 (d, J = 6.4 |
| | | | | Hz, 3H), 0.87-0.77 (m, 3H). |
| 283 | B | (2S,4R)-4-hydroxy-1-((R)-2- | 819.50 | 1H NMR (400 MHz, DMSO-d6) δ |
| | | (3-(2-((S)-3-(3-(2- | | 14.10 (d, J = 10.3 Hz, 1H), 12.39 (s, |
| | | hydroxyphenyl)-7H- | | 1H), 8.98 (s, 1H), 8.75 (d, J = 9.5 |
| | | pyrrolo[2,3-c]pyridazin-5- | | Hz, 1H), 8.40 (d, J = 7.6 Hz, 1H), |
| | | yl)piperidin-1- | | 8.18 (dd, J = 8.3, 1.7 Hz, 1H), 7.90 |
| | | yl)ethoxy)isoxazol-5-yl)-3- | | (s, 1H), 7.46-7.41 (m, 2H), 7.39- |
| | | methylbutanoyl)-N-((S)-1-(4- | | 7.33 (m, 2H), 7.31-7.27 (m, 1H), |
| | | (4-methylthiazol-5- | | 6.99-6.92 (m, 2H), 6.11 (s, 1H), |
| | | yl)phenyl)ethyl)pyrrolidine-2- | | 5.09 (d, J = 3.7 Hz, 1H), 4.91 (t, J = |
| | | carboxamide | | 7.3 Hz, 1H), 4.36 (t, J = 7.9 Hz, 1H), |
| | | | | 4.30 (t, J = 5.9 Hz, 2H), 3.69 (dd, |
| | | | | J = 10.5, 4.4 Hz, 1H), 3.63 (d, J = 9.7 |
| | | | | Hz, 1H), 3.45 (s, 1H), 3.22 (s, 1H), |
| | | | | 3.08 (d, J = 10.6 Hz, 1H), 2.95- |
| | | | | 2.85 (m, 1H), 2.81-2.70 (m, 2H), |
| | | | | 2.45 (s, 3H), 2.31-2.15 (m, |
| | | | | 4H), 1.98 (d, J = 13.0 Hz, 2H), 1.82- |
| | | | | 1.65 (m, 3H), 1.60-1.51 (m, |
| | | | | 1H), 1.38 (t, J = 6.9 Hz, 3H), 0.95 (d, |
| | | | | J = 6.4 Hz, 3H), 0.78 (d, J = 6.6 Hz, |
| | | | | 3H) |
| 291 | B | (2S,4R)-4-hydroxy-1-((R)-2- | 819.35 | 1H NM1H NMR (400 MHz, DMSO- |
| | | (3-(2-((R)-3-(3-(2- | | d6) δ 14.14-14.07 (m, 1H), 12.62- |
| | | hydroxyphenyl)-7H- | | 11.97 (m, 1H), 8.99 (s, 1H), 8.75 |
| | | pyrrolo[2,3-c]pyridazin-5- | | (s, 1H), 8.42 (d, J = 7.7 Hz, 1H), |
| | | yl)piperidin-1- | | 8.18 (d, J = 8.4, 1.7 Hz, 1H), 7.90 |
| | | yl)ethoxy)isoxazol-5-yl)-3- | | (d, J = 2.2 Hz, 1H), 7.48-7.40 (m, |
| | | methylbutanoyl)-N-((S)-1-(4- | | 2H), 7.39-7.25 (m, 3H), 7.00- |
| | | (4-methylthiazol-5- | | 6.91 (m, 2H), 6.11 (s, 1H), 5.10 (d, |
| | | yl)phenyl)ethyl)pyrrolidine-2- | | J = 3.6 Hz, 1H), 4.90 (t, J = 7.2 Hz, |
| | | carboxamide | | 1H), 4.39-4.26 (m, 4H), 3.74- |
| | | | | 3.60 (m, 2H), 3.44 (d, J = 10.8 Hz, |
| | | | | 1H), 3.27-3.14 (m, 1H), 3.12- |
| | | | | 3.05 (m, 1H), 2.95-2.88 (m, 1H), |
| | | | | 2.80-2.71 (m, 1H), 2.45 (s, 3H), |
| | | | | 2.31-2.15 (m, 3H), 2.02-1.97 (m, |
| | | | | 2H), 1.93-1.48 (m, 5H), 1.39 (d, |
| | | | | J = 7.0 Hz, 3H), 0.95 (d, J = 6.4 Hz, |
| | | | | 3H), 0.80 (d, J = 6.7 Hz, 3H). |
| 292 | B | (2S,4R)-4-hydroxy-1-((R)-2- | 805.30 | 1H NMR (400 MHz, DMSO-d6) δ |
| | | (3-(2-((R)-3-(3-(2- | | 14.07 (d, J = 7.5 Hz, 1H), 12.31 (s, |
| | | hydroxyphenyl)-7H- | | 1H), 8.98 (s, 1H), 8.75 (s, 1H), 8.41 |
| | | pyrrolo[2,3-c]pyridazin-5- | | (d, J = 7.7 Hz, 1H), 8.09 (dd, J = |
| | | yl)pyrrolidin-1- | | 7.9, 1.6 Hz, 1H), 7.89 (s, 1H), 7.47- |
| | | yl)ethoxy)isoxazol-5-yl)-3- | | 7.41 (m, 2H), 7.39-7.34 (m, 2H), |
| | | methylbutanoyl)-N-((S)-1-(4- | | 7.29 (dddd, J = 9.0, 7.2, 5.5, 1.6 Hz, |
| | | (4-methylthiazol-5- | | 1H), 7.00-6.93 (m, 2H), 6.06 (s, |
| | | yl)phenyl)ethyl)pyrrolidine-2- | | 1H), 5.10 (d, J = 3.7 Hz, 1H), 4.91 |
| | | carboxamide | | (p, J = 7.0, 6.4 Hz, 1H), 4.40-4.31 |
| | | | | (m, 4H), 3.73-3.60 (m, 3H), 3.45 |
| | | | | (dd, J = 10.0, 6.6 Hz, 1H), 3.14 (dd, |
| | | | | J = 8.9, 7.6 Hz, 1H), 2.97-2.86 (m, |
| | | | | 2H), 2.80 (t, J = 7.1 Hz, 2H), 2.64 |
| | | | | (dd, J = 8.9, 7.1 Hz, 1H), 2.45 (d, |
| | | | | J = 2.0 Hz, 3H), 2.30-2.16 (m, 1H), |
| | | | | 2.03 (t, J = 6.5 Hz, 1H), 1.96-1.89 |
| | | | | (m, 1H), 1.78 (ddd, J = 12.7, 8.0, |
| | | | | 4.8 Hz, 1H), 1.41 (d, J =7.0 Hz, 3H), |
| | | | | 0.96 (d, J = 6.2 Hz, 3H), 0.80 (d, J = |
| | | | | 6.7 Hz, 3H). |
| 299 | A | N1-((S)-1-((2S,4R)-4- | 833.20 | 1H NMR (400 MHz, DMSO-d6) δ |
| | | hydroxy-2-((4-(4- | | 14.12 (s, 1H), 12.62 (s, 1H), 8.98 (s, |
| | | methylthiazol-5- | | 1H), 8.60-8.47 (m, 3H), 8.02 (d, |
| | | yl)benzyl)carbamoyl)pyrrolidin- | | J = 7.9 Hz, 1H), 7.87 (d, J = 9.3 Hz, |
| | | 1-yl)-3,3-dimethyl-1- | | 1H), 7.40 (q, J = 8.2 Hz, 4H), 7.29 |
| | | oxobutan-2-yl)-N6-(3-(3-(2- | | (t, J = 7.7 Hz, 1H), 6.95 (t, J = 8.9 |
| | | hydroxyphenyl)-7H- | | Hz, 2H), 6.41 (s, 1H), 5.14 (d, J = |
| | | pyrrolo[2,3-c]pyridazin-6- | | 3.6 Hz, 1H), 4.55 (d, J = 9.3 Hz, |
| | | yl)bicyclo[1.1.1]pentan-1- | | 1H), 4.44 (ddd, J = 11.6, 6.8, 3.3 |
| | | yl)adipamide | | Hz, 2H), 4.36 (s, 1H), 4.22 (dd, J = |
| | | | | 16.0, 5.5 Hz, 1H), 3.73-3.61 (m, |
| | | | | 2H), 2.43 (d, J = 9.4 Hz, 9H), 2.30- |
| | | | | 2.10 (m, 2H), 2.04 (d, J = 17.9 Hz, |
| | | | | 3H), 1.91 (ddd, J = 13.0, 8.6, 4.7 |
| | | | | Hz, 1H), 1.48 (d, J = 7.4 Hz, 4H), |
| | | | | 0.95 (s, 9H). |
| 301 | A | N1-((S)-1-((2S,4R)-4- | 819.65 | 1H NMR (400 MHz, DMSO-d6) δ |
| | | hydroxy-2-((4-(4- | | 14.13 (s, 1H), 11.75 (s, 1H), 8.98 (s, |
| | | methylthiazol-5- | | 1H), 8.58 (t, J = 6.1 Hz, 1H), 8.56- |
| | | yl)benzyl)carbamoyl)pyrrolidin- | | 8.50 (m, 2H), 8.03 (dd, J = 8.1, 1.6 |
| | | 1-yl)-3,3-dimethyl-1- | | Hz, 1H), 7.91 (d, J = 9.2 Hz, 1H), |
| | | oxobutan-2-yl)-N5-(3-(3-(2- | | 7.41 (q, J = 8.4 Hz, 4H), 7.29 (ddd, |
| | | hydroxyphenyl)-7H- | | J = 8.4, 7.1, 1.6 Hz, 1H), 7.00- |
| | | pyrrolo[2,3-c]pyridazin-6- | | 6.91 (m, 2H), 6.41 (s, 1H), 5.16 (d, |
| | | yl)bicyclo[1.1.1]pentan-1- | | J = 3.5 Hz, 1H), 4.55 (d, J = 9.3 Hz, |
| | | yl)glutaramide | | 1H), 4.51-4.39 (m, 2H), 4.36 (s, |
| | | | | 1H), 4.22 (dd, J = 15.9, 5.4 Hz, 1H), |
| | | | | 3.67 (d, J = 4.3 Hz, 2H), 2.45 (s, |
| | | | | 3H), 2.42 (s, 6H), 2.31-2.12 (m, |
| | | | | 2H), 2.15-2.00 (m, 3H), 1.96- |
| | | | | 1.85 (m, 1H), 1.71 (p, J = 7.9, 7.4 |
| | | | | Hz, 2H), 0.95 (s, 9H). |
| 304 | B | (2S,4R)-4-hydroxy-1-((R)-2- | 831.70 | 1H NMR (300 MHz, DMSO-d6) δ |
| | | (3-(2-(6-(3-(2- | | 14.05 (s, 1H), 12.34 (s, 1H), 8.99 (s, |
| | | hydroxyphenyl)-7H- | | 1H), 8.60 (s, 1H), 8.42 (d, J = 7.7 |
| | | pyrrolo[2,3-c]pyridazin-5-yl)- | | Hz, 1H), 8.12 (d, J = 7.9 Hz, 1H), |
| | | 2-azaspiro[3.3]heptan-2- | | 7.90 (s, 1H), 7.51-7.26 (m, 5H), |
| | | yl)ethoxy)isoxazol-5-yl)-3- | | 7.11-6.86 (m, 2H), 6.00 (d, J = |
| | | methylbutanoyl)-N-((S)-1-(4- | | 48.9 Hz, 1H), 5.11 (d, J = 3.6 Hz, |
| | | (4-methylthiazol-5- | | 1H), 5.05-4.84 (m, 1H), 4.38 (t, |
| | | yl)phenyl)ethyl)pyrrolidine-2- | | J = 7.9 Hz, 1H), 4.29 (s, 1H), 4.10 (t, |
| | | carboxamide | | J = 5.4 Hz, 2H), 3.83-3.52 (m, |
| | | | | 3H), 3.45 (d, J = 10.7 Hz, 1H), 3.37 |
| | | | | (s, 2H), 3.16 (s, 2H), 2.82-2.67 |
| | | | | (m, 2H), 2.67-2.59 (m, 1H), 2.46 |
| | | | | (s, 3H), 2.37-2.15 (m, 4H), 2.13- |
| | | | | 1.96 (m, 1H), 1.86-1.70 (m, 1H), |
| | | | | 1.42 (d, J = 23.4, 7.0 Hz, 3H), 0.97 |
| | | | | (d, J = 6.3 Hz, 3H), 0.82 (d, J = 8.5 |
| | | | | Hz, 3H). |
| 221 | A | (2S,4R)-4-hydroxy-1-((R)-2- | 876.55 | 1H NMR (400 MHz, DMSO-d6) δ |
| | | (3-(2-((3-(3-(3-(2- | | 14.11 (s, 1H), 12.68 (s, 1H), 8.98 (s, |
| | | hydroxyphenyl)-7H- | | 1H), 8.61-8.56 (m, 1H), 8.42 (d, |
| | | pyrrolo[2,3-c]pyridazin-5- | | J = 7.6 Hz, 1H), 8.08-8.01 (m, 1H), |
| | | yl)azetidin-1-yl)-3- | | 7.48-7.40 (m, 2H), 7.39-7.33 (m, |
| | | oxopropyl)(methyl)ami- | | 2H), 7.33-7.25 (m, 1H), 7.00- |
| | | no)ethoxy)isoxazol-5-yl)-3- | | 6.91 (m, 2H), 6.68-6.63 (m, 1H), |
| | | methylbutanoyl)-N-((S)-1-(4- | | 6.08 (s, 1H), 5.11 (s, 1H), 4.90 (p, |
| | | (4-methylthiazol-5- | | J = 7.2 Hz, 1H), 4.58 (t, J = 8.5 Hz, |
| | | yl)phenyl)ethyl)pyrrolidine-2- | | 1H), 4.42-4.32 (m, 2H), 4.32- |
| | | carboxamide | | 4.10 (m, 5H), 4.10-4.02 (m, 1H), |
| | | | | 3.73-3.66 (m, 1H), 3.63 (d, J = 9.8 |
| | | | | Hz, 1H), 3.48-3.41 (m, 1H), 3.38 |
| | | | | (s, 1H), 2.72 (t, J = 5.8 Hz, 2H), |
| | | | | 2.71-2.62 (m, 1H), 2.45 (s, 3H), |
| | | | | 2.30-2.22 (m, 6H), 2.07-1.98 (m, |
| | | | | 1H), 1.83-1.72 (m, 1H), 1.44 (d, |
| | | | | J = 6.9 Hz, 1H), 1.37 (d, J = 7.0 Hz, |
| | | | | 2H), 0.99-0.91 (m, 3H), 0.79 (dd, |
| | | | | J = 15.4, 6.6 Hz, 3H). |
| 233 | B | (2S,4R)-4-hydroxy-1-((R)-2- | 856.60 | 1H NMR (400 MHz, DMSO-d6) δ |
| | | (3-(4-((3-(3-(2- | | 14.08 (s, 1H), 12.51 (s, 1H), 8.92 |
| | | hydroxyphenyl)-7H- | | (d, J = 2.7 Hz, 1H), 8.48 (s, 1H), |
| | | pyrrolo[2,3-c]pyridazin-6- | | 8.34 (d, J = 7.7 Hz, 1H), 7.95 (dd, |
| | | yl)bicyclo[1.1.1]pentan-1- | | J = 8.1, 1.6 Hz, 1H), 7.43-7.35 (m, |
| | | yl)methyl)piperazin-1- | | 2H), 7.30 (d, J = 8.4 Hz, 2H), 7.26- |
| | | yl)isoxazol-5-yl)-3- | | 7.19 (m, 1H), 6.93-6.84 (m, 2H), |
| | | methylbutanoyl)-N-((S)-1-(4- | | 6.31 (s, 1H), 6.08 (s, 1H), 4.99 (dd, |
| | | (4-methylthiazol-5- | | J = 42.5, 3.3 Hz, 1H), 4.91-4.78 |
| | | yl)phenyl)ethyl)pyrrolidine-2- | | (m, 1H), 4.30 (t, J = 7.8 Hz, 1H), |
| | | carboxamide | | 4.22 (s, 1H), 3.69-3.61 (m, 1H), |
| | | | | 3.51 (d, J = 9.9 Hz, 1H), 3.42-3.33 |
| | | | | (m, 1H), 3.17-3.08 (m, 4H), 2.48- |
| | | | | 2.45 (m, 6H), 2.39 (s, 3H), 2.10 (s, |
| | | | | 7H), 1.95 (t, J = 10.6 Hz, 1H), 1.78- |
| | | | | 1.66 (m, 1H), 1.43-1.26 (m, 3H), |
| | | | | 0.95-0.84 (m, 3H), 0.80-0.66 (m, |
| | | | | 3H). |
| 213 | B | (2S,4R)-4-hydroxy-1-((R)-2- | 831.01 | 1H NMR (300 MHz, DMSO-d6) δ |
| | | (3-(2-(((3-(3-(2- | | 14.20 (s, 1H), 12.63 (s, 1H), 8.99 (s, |
| | | hydroxyphenyl)-7H- | | 1H), 8.54 (s, 1H), 8.42 (d, J = 7.7 |
| | | pyrrolo[2,3-c]pyridazin-6- | | Hz, 1H), 8.02 (d, J = 7.8 Hz, 1H), |
| | | yl)bicyclo[1.1.1]pentan-1- | | 7.50-7.41 (m, 2H), 7.37 (d, J = 8.1 |
| | | yl)methyl)amino)ethoxy)isoxazol- | | Hz, 2H), 7.29 (t, J = 7.7 Hz, 1H), |
| | | 5-yl)-3-methylbutanoyl)- | | 6.97 (d, J = 8.1 Hz, 2H), 6.37 (s, |
| | | N-((S)-1-(4-(4-methylthiazol- | | 1H), 6.11 (s, 1H), 5.22-4.83 (m, |
| | | 5-yl)phenyl)ethyl)pyrrolidine- | | 2H), 4.53-4.08 (m, 4H), 3.81- |
| | | 2-carboxamide | | 3.51 (m, 2H), 3.54-3.37 (m, 2H), |
| | | | | 2.73 (s, 3H), 2.46 (s, 3H), 2.29- |
| | | | | 2.17 (m, 1H), 2.10 (s, 6H), 2.06- |
| | | | | 1.95 (m, 1H), 1.88-1.67 (m, 2H), |
| | | | | 1.42 (d, J = 23.4, 6.9 Hz, 3H), 0.96 |
| | | | | (d, J = 6.4 Hz, 3H), 0.81 (d, J = 6.8 |
| | | | | Hz, 3H). |
| 296 | A | (2S,4R)-4-hydroxy-1-((S)-2- | 777.30 | 1H NMR (400 MHz, DMSO-d6) δ |
| | | (3-((3-(3-(2-hydroxyphenyl)- | | 14.16 (s, 1H), 12.60 (s, 1H), 8.93 (s, |
| | | 7H-pyrrolo[2,3-c]pyridazin-6- | | 1H), 8.64-8.48 (m, 2H), 8.30 (d, J = |
| | | yl)bicyclo[1.1.1]pentan-1- | | 9.4 Hz, 1H), 8.14 (d, J = 1.8 Hz, |
| | | yl)amino)propanamido)-3,3- | | 1H), 8.06-7.99 (m, 1H), 7.48- |
| | | dimethylbutanoyl)-N-(4-(4- | | 7.34 (m, 4H), 7.33-7.25 (m, 1H), |
| | | methylthiazol-5- | | 6.95 (t, J = 8.6 Hz, 2H), 6.35 (d, J = |
| | | yl)benzyl)pyrrolidine-2- | | 1.7 Hz, 1H), 5.16 (s, 1H), 4.55 (d, |
| | | carboxamide | | J = 9.3 Hz, 1H), 4.49-4.31 (m, 3H), |
| | | | | 4.21 (dd, J = 15.9, 5.3 Hz, 1H), 3.67 |
| | | | | (d, J = 4.6 Hz, 3H), 2.81-2.64 (m, |
| | | | | 3H), 2.42 (s, 4H), 2.18 (s, 5H), 2.03 |
| | | | | (d, J = 9.2 Hz, 1H), 1.96-1.85 (m, |
| | | | | 1H), 0.96 (s, 9H). |
| 214 | B | (2S,4R)-4-hydroxy-1-((R)-2- | 848.60 | 1H NMR (300 MHz, DMSO-d6) δ |
| | | (3-(3-(4-((3-(2- | | 8.96 (s, 1H), 8.55 (s, 1H), 8.00 (d, |
| | | hydroxyphenyl)-7H- | | J = 7.3 Hz, 1H), 7.43 (d, J = 8.2 Hz, |
| | | pyrrolo[2,3-c]pyridazin-6- | | 2H), 7.40-7.23 (m, 3H), 6.97 (d, |
| | | yl)methyl)piperazin-1- | | J = 7.5 Hz, 2H), 6.55 (s, 1H), 6.06 (s, |
| | | yl)propoxy)isoxazol-5-yl)-3- | | 1H), 4.89 (d, J = 7.1 Hz, 1H), 4.37 |
| | | methylbutanoyl)-N-((S)-1-(4- | | (d, J = 8.2 Hz, 1H), 4.27 (s, 1H), |
| | | (4-methylthiazol-5- | | 4.17 (d, J = 6.1 Hz, 2H), 3.75 (s, |
| | | yl)phenyl)ethyl)pyrrolidine-2- | | 2H), 3.42-3.22 (m, 6H), 2.45 (s, |
| | | carboxamide | | 3H), 2.40 (d, J = 6.6 Hz, 2H), 2.10- |
| | | | | 2.20 (m, 1H), 2.0 (s, 1H), 1.86 (m, |
| | | | | 3H), 1.40 (m, J = 20.0, 6.8 Hz, 3H), |
| | | | | 1.23 (s, 3H), 1.15 (s, 1H), 0.94 (d, |
| | | | | J = 6.4 Hz, 3H), 0.82 (s, 1H), 0.78 (d, |
| | | | | J = 6.7 Hz, 3H). |
| 208 | B | (2S,4R)-4-hydroxy-1-((R)-2- | 834.85 | 1H NMR (400 MHz, Methanol-d4) δ |
| | | (3-(2-(4-((3-(2- | | 8.86 (d, J = 5.7 Hz, 1H), 8.44 (d, J = |
| | | hydroxyphenyl)-7H- | | 7.2 Hz, 1H), 7.97-7.90 (m, 1H), |
| | | pyrrolo[2,3-c]pyridazin-6- | | 7.47-7.33 (m, 4H), 7.32-7.23 (m, |
| | | yl)methyl)piperazin-1- | | 1H), 6.96 (dd, J = 8.2, 6.9 Hz, 2H), |
| | | yl)ethoxy)isoxazol-5-yl)-3- | | 6.58 (d, J = 4.1 Hz, 1H), 6.00 (s, |
| | | methylbutanoyl)-N-((S)-1-(4- | | 1H), 5.02 (d, J = 7.0 Hz, 1H), 4.58 |
| | | (4-methylthiazol-5- | | (s, 1H), 4.50 (t, J = 8.2 Hz, 1H), |
| | | yl)phenyl)ethyl)pyrrolidine-2- | | 4.41 (d, J = 15.4 Hz, 1H), 4.34 (t, |
| | | carboxamide | | J = 5.4 Hz, 2H), 3.87-3.79 (m, 3H), |
| | | | | 3.77-3.63 (m, 1H), 3.58 (dd, J = |
| | | | | 19.1, 11.6 Hz, 1H), 2.82 (t, J = 5.3 |
| | | | | Hz, 2H), 2.66-2.56 (m, 8H), 2.47 |
| | | | | (s, 3H), 2.42-2.29 (m, 1H), 2.22- |
| | | | | 2.12 (m, 1H), 2.01-1.88 (m, 1H), |
| | | | | 1.51 (d, J = 7.0 Hz, 3H), 1.04 (d, J = |
| | | | | 6.6 Hz, 3H), 0.89 (dd, J = 9.8, 6.7 |
| | | | | Hz, 3H). |
| 118 | B | (2S,4R)-4-hydroxy-1-((R)-2- | 789.20 | 1H NMR (400 MHz, Methanol-d4) δ |
| | | (3-(1-((3-(2-hydroxyphenyl)- | | 8.87 (d, J = 3.6 Hz, 1H), 8.47 (s, |
| | | 7H-pyrrolo[2,3-c]pyridazin-6- | | 1H), 7.94 (d, J = 8.2 Hz, 1H), 7.48- |
| | | yl)methyl)piperidin-4- | | 7.34 (m, 4H), 7.28 (t, J = 7.8 Hz, |
| | | yl)isoxazol-5-yl)-3- | | 1H), 6.98 (d, J = 7.8 Hz, 2H), 6.60 |
| | | methylbutanoyl)-N-((S)-1-(4- | | (s, 1H), 6.31 (s, 1H), 5.02 (s, 1H), |
| | | (4-methylthiazol-5- | | 4.50 (t, J = 8.2 Hz, 1H), 4.41 (d, J = |
| | | yl)phenyl)ethyl)pyrrolidine-2- | | 15.4 Hz, 1H), 3.85 (s, 3H), 3.78 (d, |
| | | carboxamide | | J = 9.9 Hz, 1H), 3.62 (d, J = 12.3 |
| | | | | Hz, 1H), 3.05 (d, J = 11.4 Hz, 2H), |
| | | | | 2.76 (t, J = 11.5 Hz, 1H), 2.47 (d, |
| | | | | J = 2.7 Hz, 3H), 2.76 (t, J = 11.5 Hz, |
| | | | | 1H), 2.31 (t, J = 11.1 Hz, 2H), 2.22- |
| | | | | 2.12 (m, 1H), 2.01-1.90 (m, 3H), |
| | | | | 1.84 (q, J = 13.7, 12.0 Hz, 2H), 1.55 |
| | | | | (dd, J = 27.0, 7.1 Hz, 3H), 1.06 (d, |
| | | | | J = 6.6 Hz, 3H), 0.86 (t, J = 7.9 Hz, |
| | | | | 3H). |
| 199 | B | (2S,4R)-4-hydroxy-1-((R)-2- | 790.30 | 1H NMR (300 MHz, DMSO-d6) δ |
| | | (3-(4-((3-(2-hydroxyphenyl)- | | 14.01 (br s, 1H), 12.76 (br s, 1H), |
| | | 7H-pyrrolo[2,3-c]pyridazin-6- | | 8.99 (s, 1H), 8.63 (s, 1H), 8.40 (d, |
| | | yl)methyl)piperazin-1- | | J = 7.7 Hz, 1H), 8.00 (d, J = 7.9 Hz, |
| | | yl)isoxazol-5-yl)-3- | | 1H), 7.49-7.40 (m, 2H), 7.40- |
| | | methylbutanoyl)-N-((S)-1-(4- | | 7.33 (m, 2H), 7.33-7.28 (m, 1H), |
| | | (4-methylthiazol-5- | | 7.05-6.90 (m, 2H), 6.68 (s, 1H), |
| | | yl)phenyl)ethyl)pyrrolidine-2- | | 6.18 (s, 1H), 5.11 (br s, 1H), 4.92 (t, |
| | | carboxamide | | J = 7.2 Hz, 1H), 4.36 (t, J = 7.9 Hz, |
| | | | | 1H), 4.29 (s, 1H), 4.11-3.81 (m, |
| | | | | 2H), 3.78-3.65 (m, 1H), 3.60 (d, |
| | | | | J = 9.8 Hz, 1H), 3.52-3.41 (m, 1H), |
| | | | | 3.37-3.08 (m, 4H), 2.90-2.59 (m, |
| | | | | 4H), 2.46 (s, 3H), 2.32-2.12 (m, |
| | | | | 1H), 2.11-1.93 (m, 1H), 1.86- |
| | | | | 1.72 (m, 1H), 1.38 (d, J = 7.0 Hz, |
| | | | | 3H), 0.96 (d, J = 6.3 Hz, 3H), 0.80 |
| | | | | (d, J = 6.7 Hz, 3H). |
| 203 | A | (2S,4R)-4-hydroxy-1-((R)-2- | 803.45 | 1H NMR (400 MHz, DMSO-d6) δ |
| | | (3-(1-(3-(2-hydroxyphenyl)- | | 13.61 (s, 1H), 13.02 (s, 1H), 8.98 (s, |
| | | 7H-pyrrolo[2,3-c]pyridazine- | | 1H), 8.69 (s, 1H), 8.40 (d, J = 7.7 |
| | | 6-carbonyl)piperidin-4- | | Hz, 1H), 8.05-7.99 (m, 1H), 7.49- |
| | | yl)isoxazol-5-yl)-3- | | 7.40 (m, 2H), 7.40-7.34 (m, 2H), |
| | | methylbutanoyl)-N-((S)-1-(4- | | 7.34-7.26 (m, 1H), 7.02-6.93 (m, |
| | | (4-methylthiazol-5- | | 2H), 6.87 (s, 1H), 6.40 (s, 1H), 5.09 |
| | | yl)phenyl)ethyl)pyrrolidine-2- | | (d, J = 3.6 Hz, 1H), 4.92 (p, J = 7.1 |
| | | carboxamide | | Hz, 1H), 4.52 (s, 1H), 4.37 (t, J = |
| | | | | 7.9 Hz, 1H), 4.29 (s, 1H), 4.02 (s, |
| | | | | 1H), 3.81-3.68 (m, 2H), 3.50- |
| | | | | 3.43 (m, 1H), 3.39-3.30 (m, 1H), |
| | | | | 3.17-2.94 (m, 2H), 2.46 (s, 3H), |
| | | | | 2.30-2.19 (m, 1H), 2.05-1.87 (m, |
| | | | | 3H), 1.83-1.70 (m, 3H), 1.42 (dd, |
| | | | | J = 29.8, 7.0 Hz, 3H), 1.02-0.95 |
| | | | | (m, 3H), 0.85-0.76 (m, 3H). |
| 212 | B | (2S,4R)-4-hydroxy-1-((R)-2- | 765.45 | 1H NMR (300 MHz, DMSO-d6) δ |
| | | (3-(2-(((3-(2-hydroxyphenyl)- | | 14.21 (s, 1H), 12.40 (br s, 1H), 8.99 |
| | | 7H-pyrrolo[2,3-c]pyridazin-6- | | (s, 1H), 8.58 (s, 1H), 8.42 (d, J = |
| | | yl)methyl)amino)ethoxy)isoxazol- | | 7.6 Hz, 1H), 8.07-8.01 (m, 1H), |
| | | 5-yl)-3-methylbutanoyl)- | | 7.48-7.41 (m, 2H), 7.41-7.34 (m, |
| | | N-((S)-1-(4-(4-methylthiazol- | | 2H), 7.33-7.25 (m, 1H), 7.02- |
| | | 5-yl)phenyl)ethyl)pyrrolidine- | | 6.91 (m, 2H), 6.56 (s, 1H), 6.08 (s, |
| | | 2-carboxamide | | 1H), 5.16-4.99 (m, 1H), 4.92 (t, |
| | | | | J = 7.2 Hz, 1H), 4.37 (t, J = 7.9 Hz, |
| | | | | 1H), 4.24 (t, J = 5.5 Hz, 3H), 4.03 |
| | | | | (s, 2H), 3.78-3.54 (m, 2H), 3.44 |
| | | | | (d, J = 11.1 Hz, 1H), 3.02-2.86 (m, |
| | | | | 2H), 2.46 (s, 3H), 2.12-1.97 (m, |
| | | | | 3H), 1.87-1.72 (m, 1H), 1.49- |
| | | | | 1.34 (m, 3H), 0.95 (d, J = 6.5 Hz, |
| | | | | 3H), 0.87-0.74 (m, 3H). |
| 253 | A | (2S,4R)-4-hydroxy-1-((R)-2- | 804.60 | 1H NMR (300 MHz, DMSO-d6) δ |
| | | (3-(4-(3-(2-hydroxyphenyl)- | | 9.00 (s, 1H), 8.79 (d, J = 5.7 Hz, |
| | | 7H-pyrrolo[2,3-c]pyridazine- | | 1H), 8.41 (d, J = 7.7 Hz, 1H), 7.88 |
| | | 6-carbonyl)piperazin-1- | | (s, 1H), 7.53-7.26 (m, 5H), 7.16- |
| | | yl)isoxazol-5-yl)-3- | | 6.92 (m, 4H), 6.18 (d, J = 35.0 Hz, |
| | | methylbutanoyl)-N-((S)-1-(4- | | 1H), 5.13-4.74 (m, 1H), 4.54- |
| | | (4-methylthiazol-5- | | 4.19 (m, 3H), 3.91-3.69 (m,6H), |
| | | yl)phenyl)ethyl)pyrrolidine-2- | | 3.36-3.29 (m, 5H), 2.46 (s, 4H), |
| | | carboxamide | | 2.10-1.99 (m, 1H), 1.85-1.75 (m, |
| | | | | 1H), 1.43 (dd, J = 25.5, 7.0 Hz, 3H), |
| | | | | 0.97 (d, J = 6.4 Hz, 3H), 0.83 (dd, |
| | | | | J = 10.7, 6.6 Hz, 3H). |
| 138 | B | (2S,4R)-4-hydroxy-1-((R)-2- | 761.60 | 1H NMR (400 MHz, Methanol-d4) δ |
| | | (3-(1-((3-(2-hydroxyphenyl)- | | 8.86 (d, J = 10.6 Hz, 1H), 8.46 (d, |
| | | 7H-pyrrolo[2,3-c]pyridazin-6- | | J = 5.8 Hz, 1H), 7.94 (d, J = 8.0 Hz, |
| | | yl)methyl)azetidin-3- | | 1H), 7.43 (d, J = 4.0 Hz, 4H), 7.28 |
| | | yl)isoxazol-5-yl)-3- | | (t, J = 7.3 Hz, 1H), 6.97 (t, J = 7.4 |
| | | methylbutanoyl)-N-((S)-1-(4- | | Hz, 2H), 6.59 (d, J = 3.7 Hz, 1H), |
| | | (4-methylthiazol-5- | | 6.48 (s, 1H), 5.04 (d, J = 7.0 Hz, |
| | | yl)phenyl)ethyl)pyrrolidine-2- | | 1H), 4.51 (t, J = 8.2 Hz, 1H), 4.42 |
| | | carboxamide | | (d, J = 17.3 Hz, 1H), 3.96 (d, J = 3.1 |
| | | | | Hz, 2H), 3.89-3.72 (m, 5H), 3.62 |
| | | | | (d, J = 10.3 Hz, 1H), 3.48 (s, 2H), |
| | | | | 2.46 (d, J = 12.6 Hz, 4H), 2.23- |
| | | | | 2.13 (m, 1H), 1.96 (ddd, J = 13.3, |
| | | | | 8.9, 4.5 Hz, 1H), 1.55 (dd, J = 19.2, |
| | | | | 7.0 Hz, 3H), 1.07 (d, J = 6.6 Hz, |
| | | | | 3H), 0.87 (d, J = 6.8 Hz, 3H). |
| 101 | A | (2S,4R)-4-hydroxy-1-((R)-2- | 870.20 | 1H NMR (400 MHz, DMSO-d6) δ |
| | | (3-((1R,4R)-5-((R)-3-(2- | | 14.49-14.34 (m, 1H), 12.24 (s, |
| | | hydroxyphenyl)-6,7,8,9- | | 1H), 8.99 (s, 1H), 8.59-8.46 (m, |
| | | tetrahydro-5H- | | 1H), 8.44-8.37 (m, 1H), 8.12- |
| | | pyridazino[3,4-b]indole-6- | | 8.02 (m, 1H), 7.49-7.40 (m, 2H), |
| | | carbonyl)-2,5- | | 7.41-7.33 (m, 2H), 7.30-7.24 (m, |
| | | diazabicyclo[2.2.1]heptan-2- | | 1H), 6.99-6.91 (m, 2H), 6.13- |
| | | yl)isoxazol-5-yl)-3- | | 6.08 (m, 1H), 5.11 (dd, J = 8.9, 3.6 |
| | | methylbutanoyl)-N-((S)-1-(4- | | Hz, 1H), 4.96-4.77 (m, 2H), 4.52- |
| | | (4-methylthiazol-5- | | 4.35 (m, 2H), 4.29 (s, 1H), 3.76- |
| | | yl)phenyl)ethyl)pyrrolidine-2- | | 3.69 (m, 1H), 3.67-3.57 (m, 1H), |
| | | carboxamide | | 3.56-3.39 (m, 3H), 3.37-3.34 (m, |
| | | | | 1H), 3.29-3.19 (m, 1H), 3.17- |
| | | | | 2.98 (m, 1H), 2.97-2.63 (m, 4H), |
| | | | | 2.46 (s, 3H), 2.34-2.20 (m, 1H), |
| | | | | 2.15-1.84 (m, 4H), 1.84-1.74 (m, |
| | | | | 2H), 1.42 (dd, J = 31.8, 6.9 Hz, 3H), |
| | | | | 1.01-0.94 (m, 3H), 0.83 (dd, J = |
| | | | | 13.7, 6.6 Hz, 3H). |
| 102 | A | (2S,4R)-4-hydroxy-1-((R)-2- | 870.40 | 1H NMR (300 MHz, DMSO-d6) δ |
| | | (3-((1S,4S)-5-((R)-3-(2- | | 14.51 (s, 1H), 12.6-11.8 (s, 1H), |
| | | hydroxyphenyl)-6,7,8,9- | | 9.06 (d, J = 1.4 Hz, 1H), 8.90-8.3 |
| | | tetrahydro-5H- | | (m, J = 21.3 Hz, 2H), 8.13 (t, J = 9.0 |
| | | pyridazino[3,4-b]indole-6- | | Hz, 1H), 7.45 (m, J = 23.6, 18.0, |
| | | carbonyl)-2,5- | | 10.3 Hz, 5H), 7.03 (m, J = 8.0, 4.4 |
| | | diazabicyclo[2.2.1]heptan-2- | | Hz, 2H), 6.18 (d, J = 7.8 Hz, 1H), |
| | | yl)isoxazol-5-yl)-3- | | 5.17 (d, J = 10.0 Hz, 1H), 5.05- |
| | | methylbutanoyl)-N-((S)-1-(4- | | 4.86 (m, 2H), 4.65-4.22 (m, 3H), |
| | | (4-methylthiazol-5- | | 3.80-3.49 (m, 5H), 3.36-3.21 (m, |
| | | yl)phenyl)ethyl)pyrrolidine-2- | | 1H), 3.2 (m, 1H), 3.00 (d, 3H), 2.83 |
| | | carboxamide | | (d, J = 12.6 Hz, 2H), 2.53 (d, J = 3.6 |
| | | | | Hz, 3H), 2.23-1.80 (m, 6H), 1.54- |
| | | | | 1.38 (m, 3H), 1.03 (m, J = 11.8, 6.4 |
| | | | | Hz, 3H), 0.88 (m, J = 9.9, 6.7 Hz, |
| | | | | 3H). |
| 157 | A | (2S,4R)-4-hydroxy-1-[(2R*)- | 829.65 | 1H NMR (300 MHz, Methanol-d4) δ |
| | | 2-(3-{1-[(6R**)-3-(2- | | 8.89 (s, 1H), 8.42-8.32 (m, 1H), |
| | | hydroxyphenyl)- | | 7.95 (d, J = 7.9 Hz, 1H), 7.52-7.36 |
| | | 5H,6H,7H,8H,9H- | | (m, 4H), 7.36-7.24 (m, 1H), 7.00 |
| | | pyridazino[3,4-b]indole-6- | | (d, J = 7.8 Hz, 2H), 6.50 (s, 1H), |
| | | carbonyl]azetidin-3-yl}-1,2- | | 5.11-5.01 (m, 1H), 4.81-4.70 (m, |
| | | oxazol-5-yl)-3- | | 1H), 4.58-4.40 (m, 4H), 4.23- |
| | | methylbutanoyl]-N-[(1S)-1- | | 3.97 (m, 2H), 3.92-3.82 (m, 2H), |
| | | [4-(4-methyl-1,3-thiazol-5- | | 3.71-3.60 (m, 1H), 3.11-2.96 (m, |
| | | yl)phenyl]ethyl]pyrrolidine-2- | | 3H), 2.86-2.82 (m, 2H), 2.54- |
| | | carboxamide | | 2.36 (m, 4H), 2.30-2.15 (m, 2H), |
| | | | | 2.05-1.94 (m, 2H), 1.58 (dt, J = |
| | | | | 26.4, 6.9 Hz, 3H), 1.11 (d, J = 6.5 |
| | | | | Hz, 3H), 0.91 (d, J = 6.7 Hz, 3H). |
| 158 | A | (2S,4R)-4-hydroxy-1-[(2R)-2- | 829.30 | 1H NMR (300 MHz, DMSO-d6) δ |
| | | (3-{1-[(6R)-3-(2- | | 14.42 (s, 1H), 12.24 (br s, 1H), 9.02 |
| | | hydroxyphenyl)- | | (s, 1H), 8.61-8.47 (m, 1H), 8.40 |
| | | 5H,6H,7H,8H,9H- | | (d, J = 7.6 Hz, 1H), 8.14-7.91 (m, |
| | | pyridazino[3,4-b]indole-6- | | 1H), 7.51-7.16 (m, 5H), 7.03- |
| | | carbonyl]azetidin-3-yl}-1,2- | | 6.84 (m, 2H), 6.47 (d, J = 7.3 Hz, |
| | | oxazol-5-yl)-3- | | 1H), 5.13-4.99 (m, 1H), 4.98- |
| | | methylbutanoyl]-N-[(1S)-1- | | 4.83 (m, 1H), 4.75-4.58 (m, 1H), |
| | | [4-(4-methyl-1,3-thiazol-5- | | 4.44-4.21 (m, 4H), 4.05-3.89 (m, |
| | | yl)phenyl]ethyl]pyrrolidine-2- | | 2H), 3.87-3.53 (m, 2H), 3.55- |
| | | carboxamide | | 3.43 (m, 1H), 2.99-2.86 (m, 3H), |
| | | | | 2.85-2.69 (m, 2H), 2.46 (s, 3H), |
| | | | | 2.36-2.18 (m, 1H), 2.15-1.96 (m, |
| | | | | 2H), 1.96-1.70 (m, 2H), 1.52- |
| | | | | 1.29 (m, 3H), 0.99 (d, J = 6.7 Hz, |
| | | | | 3H), 0.88-0.73 (m, 3H). |
| 166 | A | (2S,4R)-4-hydroxy-1-((R)-2- | 858.10 | 1H NMR (400 MHz, DMSO-d6) δ |
| | | (3-(1-((R)-3-(2- | | 14.45 (s, 1H), 12.24 (s, 1H), 8.98 (s, |
| | | hydroxyphenyl)-6,7,8,9- | | 1H), 8.56 (d, J = 11.6 Hz, 1H), 8.40 |
| | | tetrahydro-5H- | | (d, J = 7.7 Hz, 1H), 8.08 (d, J = 8.0 |
| | | pyridazino[3,4-b]indole-6- | | Hz, 1H), 7.44 (d, J = 8.3 Hz, 2H), |
| | | carbonyl)piperidin-4- | | 7.37 (d, J = 8.1 Hz, 2H), 7.28 (t, J = |
| | | yl)isoxazol-5-yl)-3- | | 7.6 Hz, 1H), 6.95 (d, J = 7.9 Hz, |
| | | methylbutanoyl)-N-((S)-1-(4- | | 2H), 6.38 (s, 1H), 5.10 (s, 1H), 4.92 |
| | | (4-methylthiazol-5- | | (t, J = 7.1 Hz, 1H), 4.48 (s, 1H), |
| | | yl)phenyl)ethyl)pyrrolidine-2- | | 4.37 (t, J = 7.9 Hz, 1H), 4.30 (s, |
| | | carboxamide | | 2H), 4.11 (s, 1H), 3.80-3.68 (m, |
| | | | | 2H), 3.47 (d, J = 10.6 Hz, 1H), 3.21 |
| | | | | (d, J = 27.9 Hz, 2H), 3.06-2.90 (m, |
| | | | | 4H), 2.78 (s, 2H), 2.46 (s, 3H), 2.31- |
| | | | | 2.21 (m, 2H), 2.08-1.86 (m, 6H), |
| | | | | 1.79 (ddd, J = 12.8, 8.2, 4.9 Hz, |
| | | | | 1H), 1.64 (s, 1H), 1.38 (d, J = 7.0 |
| | | | | Hz, 3H), 0.98 (d, J = 6.5 Hz, 3H), |
| | | | | 0.79 (d, J = 6.6 Hz, 3H). |
| 167 | A | (S)-N-(2-((5-((R)-1-((2S,4R)- | 833.50 | 1H NMR (300 MHz, Methanol-d4) δ |
| | | 4-hydroxy-2-(((S)-1-(4-(4- | | 8.88 (d, J = 6.6 Hz, 1H), 8.36 (d, J = |
| | | methylthiazol-5- | | 5.8 Hz, 1H), 7.99 (dd, J = 8.3, 1.7 |
| | | yl)phenyl)ethyl)carbamoyl)pyrrolidin- | | Hz, 1H), 7.56-7.33 (m, 4H), 7.29 |
| | | 1-yl)-3-methyl-1- | | (td, J = 7.6, 1.5 Hz, 1H), 7.05-6.92 |
| | | oxobutan-2-yl)isoxazol-3- | | (m, 2H), 6.03 (d, J = 12.6 Hz, 1H), |
| | | yl)oxy)ethyl)-3-(2- | | 5.11-4.97 (m, 1H), 4.53 (t, J = 8.2 |
| | | hydroxyphenyl)-6,7,8,9- | | Hz, 1H), 4.49-4.29 (m, 3H), 3.86 |
| | | tetrahydro-5H- | | (dd, J = 10.8, 4.1 Hz, 1H), 3.75- |
| | | pyridazino[3,4-b]indole-6- | | 3.59 (m, 4H), 3.18-2.86 (m, 4H), |
| | | carboxamide | | 2.86-2.70 (m, 1H), 2.48 (d, J = 8.6 |
| | | | | Hz, 3H), 2.44-2.30 (m, 1H), 2.29- |
| | | | | 1.91 (m, 4H), 1.56 (dd, J = 23.1, 7.0 |
| | | | | Hz, 3H), 1.07 (d, J = 6.6 Hz, 3H), |
| | | | | 0.99-0.88 (m, 3H). |
| 172 | A | (R)-N-(2-((5-((R)-1-((2S,4R)- | 833.20 | 1H NMR (400 MHz, DMSO-d6) δ |
| | | 4-hydroxy-2-(((S)-1-(4-(4- | | 14.43 (d, J = 3.2 Hz, 1H), 12.25 (s, |
| | | methylthiazol-5- | | 1H), 8.99 (s, 1H), 8.55 (d, J = 2.1 |
| | | yl)phenyl)ethyl)carbamoyl)pyrrolidin- | | Hz, 1H), 8.42 (d, J = 7.7 Hz, 1H), |
| | | 1-yl)-3-methyl-1- | | 8.29 (t, J = 5.3 Hz, 1H), 8.08 (td, J = |
| | | oxobutan-2-yl)isoxazol-3- | | 7.8, 1.5 Hz, 1H), 7.48-7.41 (m, |
| | | yl)oxy)ethyl)-3-(2- | | 2H), 7.39-7.33 (m, 2H), 7.31- |
| | | hydroxyphenyl)-6,7,8,9- | | 7.23 (m, 1H), 6.94 (t, J = 8.3 Hz, |
| | | tetrahydro-5H- | | 2H), 6.12 (s, 1H), 5.12 (d, J = 3.6 |
| | | pyridazino[3,4-b]indole-6- | | Hz, 1H), 4.97-4.84 (m, 1H), 4.37 |
| | | carboxamide | | (t, J = 7.9 Hz, 1H), 4.32-4.18 (m, |
| | | | | 3H), 3.74-3.62 (m, 2H), 3.54- |
| | | | | 3.43 (m, 3H), 3.01-2.83 (m, 3H), |
| | | | | 2.82-2.72 (m, 1H), 2.71-2.64 (m, |
| | | | | 1H), 2.45 (d, J = 3.5 Hz, 3H), 2.31- |
| | | | | 2.16 (m, 1H), 2.14-1.98 (m, 2H), |
| | | | | 1.96-1.84 (m, 1H), 1.82-1.74 (m, |
| | | | | 1H), 1.42 (dd, J = 35.8, 6.9 Hz, 3H), |
| | | | | 0.96 (d, J = 6.3 Hz, 3H), 0.82 (dd, |
| | | | | J = 13.7, 6.6 Hz, 3H). |
| 188 | A | (2S,4R)-4-hydroxy-1-((R)-2- | 858.50 | 1H NMR (400 MHz, DMSO-d6) δ |
| | | (3-(4-((S)-3-(2- | | 14.43 (s, 1H), 12.26 (s, 1H), 8.99 (s, |
| | | hydroxyphenyl)-6,7,8,9- | | 1H), 8.56 (d, J = 2.9 Hz, 1H), 8.41 |
| | | tetrahydro-5H- | | (d, J = 7.6 Hz, 1H), 8.08 (dd, J = |
| | | pyridazino[3,4-b]indole-6- | | 8.0, 1.7 Hz, 1H), 7.44 (d, J = 8.3 |
| | | carbonyl)piperazin-1- | | Hz, 2H), 7.37 (d, J = 8.1 Hz, 2H), |
| | | yl)isoxazol-5-yl)-3- | | 7.31-7.25 (m, 1H), 6.94 (t, J = 7.8 |
| | | methylbutanoyl)-N-((S)-1-(4- | | Hz, 2H), 6.23 (s, 1H), 5.12 (d, J = |
| | | (4-methylthiazol-5- | | 3.6 Hz, 1H), 4.92 (t, J = 7.1 Hz, 1H), |
| | | yl)phenyl)ethyl)pyrrolidine-2- | | 4.37 (t, J = 7.9 Hz, 1H), 4.29 (s, |
| | | carboxamide | | 1H), 3.79-3.64 (m, 4H), 3.60 (t, |
| | | | | J = 9.4 Hz, 2H), 3.62-3.58 (m, 1H), |
| | | | | 3.28-3.17 (m, 4H), 2.95 (d, J = |
| | | | | 15.0 Hz, 3H), 2.82-2.71 (m, 2H), |
| | | | | 2.46 (s, 3H), 2.24 (d, J = 21.2 Hz, |
| | | | | 1H), 2.07 (d, J = 6.8 Hz, 2H), 1.95- |
| | | | | 1.89 (m, 1H), 1.79 (td, J = 8.1, 4.1 |
| | | | | Hz, 1H), 1.47-1.35 (m, 3H), 0.97 |
| | | | | (t, J = 6.6 Hz, 3H), 0.81 (t, J = 7.3 |
| | | | | Hz, 3H). |
| 192 | A | (2S,4R)-4-hydroxy-1-((R)-2- | 858.30 | 1H NMR (400 MHz, DMSO-d6) δ |
| | | (3-(4-((R)-3-(2- | | 14.43 (s, 1H), 12.25 (s, 1H), 8.99 (s, |
| | | hydroxyphenyl)-6,7,8,9- | | 1H), 8.56 (s, 1H), 8.39 (d, J = 7.7 |
| | | tetrahydro-5H- | | Hz, 1H), 8.08 (d, J = 8.2 Hz, 1H), |
| | | pyridazino[3,4-b]indole-6- | | 7.49-7.41 (m, 2H), 7.37 (d, J = 8.3 |
| | | carbonyl)piperazin-1- | | Hz, 2H), 7.32-7.24 (m, 1H), 7.00- |
| | | yl)isoxazol-5-yl)-3- | | 6.91 (m, 2H), 6.23 (s, 1H), 5.11 (d, |
| | | methylbutanoyl)-N-((S)-1-(4- | | J = 3.7 Hz, 1H), 4.97-4.87 (m, |
| | | (4-methylthiazol-5- | | 1H), 4.37 (t, J = 7.8 Hz, 1H), 4.33- |
| | | yl)phenyl)ethyl)pyrrolidine-2- | | 4.25 (m, 1H), 3.80-3.64 (m, 4H), |
| | | carboxamide | | 3.64-3.53 (m, 2H), 3.48-3.40 (m, |
| | | | | 1H), 3.29-3.10 (m, 4H), 3.05- |
| | | | | 2.86 (m, 3H), 2.83-2.71 (m, 1H), |
| | | | | 2.46 (s, 3H), 2.31-2.14 (m, 2H), |
| | | | | 2.12-1.98 (m, 2H), 1.98-1.85 (m, |
| | | | | 1H), 1.84-1.74 (m, 1H), 1.38 (d, |
| | | | | J = 7.0 Hz, 3H), 0.97 (t, J = 6.7 Hz, |
| | | | | 3H), 0.82 (d, J = 6.7 Hz, 3H). |
| 195 | A | (R)-N-(6-(((S)-1-((2S,4R)-4- | 835.60 | 1H NMR (300 MHz, Methanol-d4) δ |
| | | hydroxy-2-((4-(4- | | 8.86 (s, 1H), 8.32 (s, 1H), 7.97- |
| | | methylthiazol-5- | | 7.88 (m, 1H), 7.47-7.38 (m, 4H), |
| | | yl)benzyl)carbamoyl)pyrrolidin- | | 7.31-7.26 (m, 1H), 6.97 (td, J = |
| | | 1-yl)-3,3-dimethyl-1- | | 7.8, 7.2, 1.3 Hz, 2H), 4.66-4.50 |
| | | oxobutan-2-yl)amino)-6- | | (m, 4H), 4.35 (d, J = 15.5 Hz, 1H), |
| | | oxohexyl)-3-(2- | | 3.94-3.90 (m, 1H), 3.82-3.77 (m, |
| | | hydroxyphenyl)-6,7,8,9- | | 1H), 3.26 (td, J = 6.9, 2.6 Hz, 2H), |
| | | tetrahydro-5H- | | 3.04-2.84 (m, 4H), 2.76-2.67 (m, |
| | | pyridazino[3,4-b]indole-6- | | 1H), 2.46 (s, 3H), 2.38-2.20 (m, |
| | | carboxamide | | 4H), 2.14-2.02 (m, 2H), 1.70- |
| | | | | 1.37 (m, 7H), 1.06 (s, 9H), 0.87- |
| | | | | 0.89 (m, 2H) . |
| 196 | A | (R)-N-(4-(((S)-1-((2S,4R)-4- | 807.50 | 1H NMR (300 MHz, Methanol-d4) δ |
| | | hydroxy-2-((4-(4- | | 8.86 (s, 1H), 8.31 (s, 1H), 7.94 (d, |
| | | methylthiazol-5- | | J = 8.0, 1.6 Hz, 1H), 7.47-7.38 (m, |
| | | yl)benzyl)carbamoyl)pyrrolidin- | | 4H), 7.31-7.25 (m, 1H), 7.00- |
| | | 1-yl)-3,3-dimethyl-1- | | 6.93 (m, 2H), 4.66-4.51 (m, 4H), |
| | | oxobutan-2-yl)amino)-4- | | 4.38-4.32 (m, 1H), 3.93 (d, J = |
| | | oxobutyl)-3-(2- | | 11.0 Hz, 1H), 3.85-3.81 (m, 1H), |
| | | hydroxyphenyl)-6,7,8,9- | | 3.28 (d, J = 6.9 Hz, 1H), 3.05-2.84 |
| | | tetrahydro-5H- | | (m, 4H), 2.76-2.67 (m, 1H), 2.46 |
| | | pyridazino[3,4-b]indole-6- | | (s, 3H), 2.37 (td, J = 7.3, 1.5 Hz, |
| | | carboxamide | | 2H), 2.27-2.21 (m, 2H), 2.15-2.03 |
| | | | | (m, 2H), 1.91-1.82 (m, 2H), 1.08 |
| | | | | (s, 9H). |
| 143 | B | (2S,4R)-4-hydroxy-1-((R)-2- | 819.60 | 1H NMR (300 MHz, Methanol-d4) δ |
| | | (3-(3-(((S)-3-(2- | | 8.95 (d, J = 5.6 Hz, 1H), 8.44 (d, J = |
| | | hydroxyphenyl)-6,7,8,9- | | 4.6 Hz, 1H), 8.11-8.02 (m, 1H), |
| | | tetrahydro-5H- | | 7.52 (d, J = 3.1 Hz, 3H), 7.52-7.37 |
| | | pyridazino[3,4-b]indol-6- | | (m, 1H), 7.37 (t, J = 7.7 Hz, 1H), |
| | | yl)amino)propoxy)isoxazol-5- | | 7.11-7.01 (m, 2H), 6.07 (d, J = |
| | | yl)-3-methylbutanoyl)-N-((S)- | | 13.0 Hz, 1H), 5.12 (d, J = 7.0 Hz, |
| | | 1-(4-(4-methylthiazol-5- | | 1H), 4.60 (t, J = 8.2 Hz, 1H), 4.53 |
| | | yl)phenyl)ethyl)pyrrolidine-2- | | (s, 1H), 4.41 (t, J = 6.0 Hz, 2H), |
| | | carboxamide | | 3.98-3.82 (m, 1H), 3.73 (m, J = |
| | | | | 16.9, 10.4 Hz, 2H), 3.66-3.52 (m, |
| | | | | 0H), 3.28 (d, J = 13.4 Hz, 2H), 3.11- |
| | | | | 3.00 (m, 4H), 2.65 (s, 1H), 2.55 |
| | | | | (d, J = 8.5 Hz, 3H), 2.48-2.23 (m, |
| | | | | 3H), 2.21-1.85 (m, 4H), 1.63 (m, |
| | | | | J = 17.0, 7.0 Hz, 3H), 1.40 (d, J = |
| | | | | 11.8 Hz, 2H), 1.14 (d, J = 6.6 Hz, |
| | | | | 3H), 0.98 (t, J = 7.0 Hz, 3H). |
| 144 | A | 1-(5-((R)-1-((2S,4R)-4- | 872.60 | 1H NMR (300 MHz, DMSO-d6) δ |
| | | hydroxy-2-(((S)-1-(4-(4- | | 14.44 (s, 1H), 12.26 (s, 1H), 8.99 (s, |
| | | methylthiazol-5- | | 1H), 8.55 (s, 1H), 8.40 (d, J = 7.7 |
| | | yl)phenyl)ethyl)carbamoyl)pyrrolidin- | | Hz, 1H), 8.10 (d, J = 7.8 Hz, 1H), |
| | | 1-yl)-3-methyl-1- | | 8.02 (d, J = 7.6 Hz, 1H), 7.45 (d, J = |
| | | oxobutan-2-yl)isoxazol-3-yl)- | | 8.1 Hz, 2H), 7.37 (d, J = 8.2 Hz, |
| | | N-((S)-3-(2-hydroxyphenyl)- | | 2H), 7.28 (t, J = 7.5 Hz, 1H), 6.96 |
| | | 6,7,8,9-tetrahydro-5H- | | (d, J = 7.9 Hz, 2H), 6.15 (s, 1H), |
| | | pyridazino[3,4-b]indol-6- | | 5.11 (d, J = 3.7 Hz, 1H), 5.03-4.87 |
| | | yl)piperidine-4-carboxamide | | (m, 1H), 4.37 (t, J = 7.8 Hz, 1H), |
| | | | | 4.29 (s, 1H), 4.13 (s, 2H), 3.70 (t, |
| | | | | J = 11.7 Hz, 3H), 3.58 (d, J = 9.9 Hz, |
| | | | | 1H), 3.45 (s, 1H), 3.12-3.00 (m, |
| | | | | 1H), 2.94 (s, 2H), 2.79 (d, J = 12.5 |
| | | | | Hz, 2H), 2.59 (d, J = 8.2 Hz, 1H), |
| | | | | 2.46 (s, 3H), 2.37-2.13 (m, 2H), |
| | | | | 2.05-1.85 (m, 4H), 1.74-1.57 (m, |
| | | | | 4H), 1.39 (d, J = 7.0 Hz, 3H), 0.96 |
| | | | | (d, J = 6.4 Hz, 3H), 0.80 (d, J = 6.6 |
| | | | | Hz, 3H). |
| 145 | B | (2S,4R)-4-hydroxy-1-((R)-2- | 819.60 | 1H NMR (300 MHz, Methanol-d4) δ |
| | | (3-(3-(((R)-3-(2- | | 8.95 (d, J = 6.9 Hz, 1H), 8.43 (d, J = |
| | | hydroxyphenyl)-6,7,8,9- | | 8.3 Hz, 1H), 8.06 (m, J = 8.3, 1.6 |
| | | tetrahydro-5H- | | Hz, 1H), 7.52 (d, J = 3.2 Hz, 4H), |
| | | pyridazino[3,4-b]indol-6- | | 7.43-7.31 (m, 1H), 7.11-7.01 (m, |
| | | yl)amino)propoxy)isoxazol-5- | | 2H), 6.07 (d, J = 14.8 Hz, 1H), 5.12 |
| | | yl)-3-methylbutanoyl)-N-((S)- | | (d, J = 7.0 Hz, 1H), 4.68-4.55 (m, |
| | | 1-(4-(4-methylthiazol-5- | | 1H), 4.41 (t, J = 6.1 Hz, 3H), 3.92 |
| | | yl)phenyl)ethyl)pyrrolidine-2- | | (m, J = 10.9, 4.2 Hz, 1H), 3.76 (d, |
| | | carboxamide | | J = 9.9 Hz, 2H), 3.69 (d, J = 10.9 Hz, |
| | | | | 0H), 3.28 (d, J = 12.2 Hz, 2H), 3.07 |
| | | | | (s, 5H), 2.74-2.62 (m, 1H), 2.55 |
| | | | | (d, J = 10.3 Hz, 3H), 2.39 (s, 2H), |
| | | | | 2.31-1.88 (m, 6H), 1.63 (m, J = |
| | | | | 18.2, 7.0 Hz, 3H), 1.40 (d, J = 12.4 |
| | | | | Hz, 2H), 1.14 (d, J = 6.5 Hz, 3H), |
| | | | | 0.97 (d, J = 6.6 Hz, 3H). |
| 146 | A | 1-(5-((R)-1-((2S,4R)-4- | 872.55 | 1H NMR (300 MHz, DMSO-d6) δ |
| | | hydroxy-2-(((S)-1-(4-(4- | | 14.45 (s, 1H), 12.26 (s, 1H), 8.99 (s, |
| | | methylthiazol-5- | | 1H), 8.55 (s, 1H), 8.40 (d, J = 7.7 |
| | | yl)phenyl)ethyl)carbamoyl)pyrrolidin- | | Hz, 1H), 8.15-8.06 (m, 1H), 8.01 |
| | | 1-yl)-3-methyl-1- | | (d, J = 7.6 Hz, 1H), 7.52-7.33 (m, |
| | | oxobutan-2-yl)isoxazol-3-yl)- | | 4H), 7.28 (t, J = 7.9 Hz, 1H), 6.96 |
| | | N-((R)-3-(2-hydroxyphenyl)- | | (d, J = 7.8 Hz, 2H), 6.15 (s, 1H), |
| | | 6,7,8,9-tetrahydro-5H- | | 5.11 (d, J = 3.7 Hz, 1H), 5.03-4.87 |
| | | pyridazino[3,4-b]indol-6- | | (m, 1H), 4.37 (t, J = 7.8 Hz, 1H), |
| | | yl)piperidine-4-carboxamide | | 4.29 (s, 1H), 4.14 (s, 1H), 3.70 (t, |
| | | | | J = 11.8 Hz, 3H), 3.58 (d, J = 9.9 Hz, |
| | | | | 1H), 3.43 (d, J = 10.4 Hz, 1H), 3.07 |
| | | | | (dd, J = 15.4, 5.2 Hz, 1H), 2.94 (s, |
| | | | | 2H), 2.79 (d, J = 12.3 Hz, 2H), 2.59 |
| | | | | (d, J = 8.0 Hz, 1H), 2.46 (s, 3H), |
| | | | | 2.39-2.14 (m, 2H), 2.07-1.85 (m, |
| | | | | 4H), 1.81-1.57 (m, 4H), 1.42 (dd, |
| | | | | J = 23.3, 7.0 Hz, 3H), 0.96 (d, J = |
| | | | | 6.5 Hz, 3H), 0.80 (d, J = 6.6 Hz, |
| | | | | 3H). |
| 150 | A | (2S,4R)-4-hydroxy-1-((R)-2- | 819.60 | 1H NMR (400 MHz, Methanol-d4) δ |
| | | (3-(2-(((R)-3-(2- | | 8.86 (d, J = 10.7 Hz, 1H), 8.29 (d, |
| | | hydroxyphenyl)-6,7,8,9- | | J = 27.0 Hz, 1H), 7.91 (dd, J = 31.7, |
| | | tetrahydro-5H- | | 7.9 Hz, 1H), 7.50-7.32 (m, 4H), |
| | | pyridazino[3,4-b]indol-6- | | 7.31-7.18 (m, 1H), 6.96 (dd, J = |
| | | yl)amino)-2- | | 8.0, 6.8 Hz, 2H), 6.06 (d, J = 17.2 |
| | | oxoethoxy)isoxazol-5-yl)-3- | | Hz, 1H), 5.03 (q, J = 6.9 Hz, 1H), |
| | | methylbutanoyl)-N-((S)-1-(4- | | 4.74 (d, J = 8.7 Hz, 2H), 4.53 (t, J = |
| | | (4-methylthiazol-5- | | 8.2 Hz, 1H), 4.40 (d, J = 36.6 Hz, |
| | | yl)phenyl)ethyl)pyrrolidine-2- | | 1H), 3.84 (dd, J = 10.8, 4.1 Hz, 1H), |
| | | carboxamide | | 3.69 (d, J = 9.8 Hz, 1H), 3.63 (d, J = |
| | | | | 11.2 Hz, 1H), 3.13 (dd, J = 15.3, 5.4 |
| | | | | Hz, 1H), 2.97 (d, J = 6.1 Hz, 2H), |
| | | | | 2.84-2.64 (m, 1H), 2.47 (s, 2H), |
| | | | | 2.44-2.31 (m, 2H), 2.26-2.01 (m, |
| | | | | 3H), 1.96 (ddd, J = 13.2, 8.8, 4.5 |
| | | | | Hz, 1H), 1.51 (d, J = 7.0 Hz, 2H), |
| | | | | 1.45 (s, 1H), 1.05 (d, J = 6.5 Hz, |
| | | | | 3H), 0.90 (dd, J = 9.2, 6.7 Hz, 3H). |
| 159 | B | (2S,4R)-4-hydroxy-1-((R)-2- | 858.50 | 1H NMR (400 MHz, Methanol-d4) δ |
| | | (3-(4-((((S)-3-(2- | | 8.86 (d, J = 5.4 Hz, 1H), 8.52 (s, |
| | | hydroxyphenyl)-6,7,8,9- | | 1H), 8.32 (s, 1H), 7.93 (d, J = 7.8 |
| | | tetrahydro-5H- | | Hz, 1H), 7.50-7.35 (m, 4H), 7.28 |
| | | pyridazino[3,4-b]indol-6- | | (t, J = 7.7 Hz, 1H), 6.98 (d, J = 7.9 |
| | | yl)amino)methyl)piperidin-1- | | Hz, 2H), 6.10 (d, J = 21.6 Hz, 1H), |
| | | yl)isoxazol-5-yl)-3- | | 5.04 (q, J = 7.2 Hz, 1H), 4.56-4.42 |
| | | methylbutanoyl)-N-((S)-1-(4- | | (m, 2H), 3.85 (dd, J = 10.8, 4.2 Hz, |
| | | (4-methylthiazol-5- | | 1H), 3.76 (d, J = 12.2 Hz, 2H), 3.63 |
| | | yl)phenyl)ethyl)pyrrolidine-2- | | (t, J = 10.5 Hz, 2H), 3.57-3.44 (m, |
| | | carboxamide | | 2H), 3.03 (s, 3H), 2.91 (t, J = 12.3 |
| | | | | Hz, 2H), 2.73 (s, 1H), 2.47 (d, J = |
| | | | | 7.2 Hz, 3H), 2.38 (dd, J = 16.5, 6.7 |
| | | | | Hz, 2H), 2.18 (t, J = 10.8 Hz, 1H), |
| | | | | 2.04 (s, 1H), 1.95 (ddd, J = 22.7, |
| | | | | 15.6, 10.5 Hz, 4H), 1.59 (d, J = 7.0 |
| | | | | Hz, 1H), 1.52 (d, J = 7.0 Hz, 3H), |
| | | | | 1.42 (s, 2H), 1.06 (d, J = 6.5 Hz, |
| | | | | 3H), 0.91 (d, J = 6.7 Hz, 3H). |
| 168 | A | (2S,4R)-4-hydroxy-1-((R)-2- | 819.30 | 1H NMR (400 MHz, Methanol-d4) δ |
| | | (3-(2-(((S)-3-(2- | | 8.83 (d, J = 30.6 Hz, 1H), 8.35 (s, |
| | | hydroxyphenyl)-6,7,8,9- | | 1H), 8.14-7.87 (m, 1H), 7.50- |
| | | tetrahydro-5H- | | 7.34 (m, 3H), 7.21 (dd, J = 40.3, 8.0 |
| | | pyridazino[3,4-b]indol-6- | | Hz, 2H), 7.03-6.81 (m, 2H), 6.08 |
| | | yl)amino)-2- | | (d, J = 13.6 Hz, 1H), 4.99 (dd, J = |
| | | oxoethoxy)isoxazol-5-yl)-3- | | 26.8, 7.2 Hz, 1H), 4.87 (d, J = 1.3 |
| | | methylbutanoyl)-N-((S)-1-(4- | | Hz, 2H), 4.51 (t, J = 8.3 Hz, 1H), |
| | | (4-methylthiazol-5- | | 4.40 (d, J = 32.5 Hz, 2H), 3.91- |
| | | yl)phenyl)ethyl)pyrrolidine-2- | | 3.43 (m, 4H), 3.01 (s, 2H), 2.81- |
| | | carboxamide | | 2.56 (m, 1H), 2.48 (d, J = 1.4 Hz, |
| | | | | 2H), 2.34 (d, J = 28.1 Hz, 2H), 2.18 |
| | | | | (s, 1H), 2.03 (dd, J = 53.1, 9.2 Hz, |
| | | | | 2H), 1.58 (d, J = 7.0 Hz, 1H), 1.51 |
| | | | | (d, J = 7.0 Hz, 2H), 1.36 (d, J = 6.4 |
| | | | | Hz, 2H), 1.05 (d, J = 6.7 Hz, 2H), |
| | | | | 0.93 (s, 2H), 0.89 (s, 1H). |
| 173 | B | (2S,4R)-4-hydroxy-1-((R)-2- | 858.50 | 1H NMR (400 MHz, Methanol-d4) δ |
| | | (3-(4-((((R)-3-(2- | | 8.86 (d, J = 11.0 Hz, 1H), 8.33 (d, |
| | | hydroxyphenyl)-6,7,8,9- | | J = 4.0 Hz, 1H), 7.96 (d, J = 7.9 Hz, |
| | | tetrahydro-5H- | | 1H), 7.49-7.23 (m, 5H), 7.03- |
| | | pyridazino[3,4-b]indol-6- | | 6.89 (m, 2H), 6.08 (d, J = 23.2 Hz, |
| | | yl)amino)methyl)piperidin-1- | | 1H), 5.03 (d, J = 7.0 Hz, 1H), 4.55- |
| | | yl)isoxazol-5-yl)-3- | | 4.42 (m, 2H), 3.84 (dd, J = 10.8, 4.2 |
| | | methylbutanoyl)-N-((S)-1-(4- | | Hz, 1H), 3.72 (d, J = 12.1 Hz, 2H), |
| | | (4-methylthiazol-5- | | 3.66-3.57 (m, 2H), 3.23-3.05 (m, |
| | | yl)phenyl)ethyl)pyrrolidine-2- | | 2H), 3.03-2.81 (m, 4H), 2.73 (s, |
| | | carboxamide | | 2H), 2.61-2.50 (m, 1H), 2.46 (d, |
| | | | | J = 10.7 Hz, 3H), 2.42-2.25 (m, 2H), |
| | | | | 2.22-2.13 (m, 1H), 2.07-1.70 (m, |
| | | | | 3H), 1.59 (d, J = 7.0 Hz, 1H), 1.52 |
| | | | | (d, J = 7.0 Hz, 3H), 1.33 (d, J = 11.4 |
| | | | | Hz, 2H), 1.28 (s, 1H), 1.05 (d, J = |
| | | | | 6.5 Hz, 3H), 0.89 (d, J = 6.7 Hz, |
| | | | | 4H). |
| 174 | A | N1-((S)-1-((2S,4R)-4- | 793.40 | 1H NMR (400 MHz, Methanol-d4) δ |
| | | hydroxy-2-((4-(4- | | 8.85 (s, 1H), 8.25 (s, 1H), 7.95- |
| | | methylthiazol-5- | | 7.88 (m, 1H), 7.46-7.38 (m, 4H), |
| | | yl)benzyl)carbamoyl)pyrrolidin- | | 7.29-7.24 (m, 1H), 6.97-6.93 (m, |
| | | 1-yl)-3,3-dimethyl-1- | | 2H), 4.62-4.50 (m, 4H), 4.36- |
| | | oxobutan-2-yl)-N4-((S)-3-(2- | | 4.32 (m, 1H), 4.26-4.24 (m, 1H), |
| | | hydroxyphenyl)-6,7,8,9- | | 3.88 (d, J = 10.9 Hz, 1H), 3.79 (dd, |
| | | tetrahydro-5H- | | J = 11.0, 3.9 Hz, 1H), 3.09 (dd, J = |
| | | pyridazino[3,4-b]indol-6- | | 15.7, 5.3 Hz, 1H), 2.99-2.97 (m, |
| | | yl)succinamide | | 2H), 2.68-2.61 (m, 2H), 2.60- |
| | | | | 2.50 (m, 3H), 2.45 (s, 3H), 2.24- |
| | | | | 1.98 (m, 4H), 1.04 (s, 9H). |
| 175 | A | N1-((S)-1-((2S,4R)-4- | 821.50 | 1H NMR (400 MHz, Methanol-d4) δ |
| | | hydroxy-2-((4-(4- | | 8.85 (s, 1H), 8.29 (s, 1H), 7.94- |
| | | methylthiazol-5- | | 7.86 (m, 1H), 7.42 (d, J = 8.2 Hz, |
| | | yl)benzyl)carbamoyl)pyrrolidin- | | 2H), 7.38-7.34 (m, 2H), 7.28- |
| | | 1-yl)-3,3-dimethyl-1- | | 7.24 (m, 1H), 6.97-6.93 (m, 2H), |
| | | oxobutan-2-yl)-N6-((S)-3-(2- | | 4.62-4.55 (m, 2H), 4.52-4.48 (m, |
| | | hydroxyphenyl)-6,7,8,9- | | 2H), 4.35-4.32 (m, 1H), 4.29- |
| | | tetrahydro-5H- | | 4.23 (m, 1H), 3.90 (d, J = 11.0 Hz, |
| | | pyridazino[3,4-b]indol-6- | | 1H), 3.80 (dd, J = 10.9, 4.0 Hz, 1H), |
| | | yl)adipamide | | 3.13 (dd, J = 15.3, 5.4 Hz, 1H), 2.98 |
| | | | | (t, J = 6.4 Hz, 2H), 2.65 (dd, J = |
| | | | | 15.4, 8.1 Hz, 1H), 2.44 (s, 3H), 2.32- |
| | | | | 1.96 (m, 9H), 1.66-1.64 (m, 4H), |
| | | | | 1.03 (s, 9H). |
| 181 | B | (2S,4R)-4-hydroxy-1-((R)-2- | 805.40 | 1H NMR (300 MHz, DMSO-d6) δ |
| | | (3-(2-(((R)-3-(2- | | 14.23 (br s, 1H), 12.40 (s, 1H), 9.00 |
| | | hydroxyphenyl)-6,7,8,9- | | (d, J = 2.3 Hz, 1H), 8.58 (s, 1H), |
| | | tetrahydro-5H- | | 8.39 (d, J = 7.6 Hz, 1H), 8.16-8.00 |
| | | pyridazino[3,4-b]indol-6- | | (m, 1H), 7.53-7.41 (m, 2H), 7.41- |
| | | yl)amino)ethoxy)isoxazol-5- | | 7.34 (m, 2H), 7.34-7.25 (m, 1H), |
| | | yl)-3-methylbutanoyl)-N-((S)- | | 7.04-6.91 (m, 2H), 6.11 (d, J = |
| | | 1-(4-(4-methylthiazol-5- | | 42.0 Hz, 1H), 5.08 (dd, J = 28.1, 3.2 |
| | | yl)phenyl)ethyl)pyrrolidine-2- | | Hz, 1H), 4.99-4.61 (m, 1H), 4.49 |
| | | carboxamide | | (s, 2H), 4.41-4.21 (m, 2H), 3.82- |
| | | | | 3.54 (m, 3H), 3.52-3.36 (m, 3H), |
| | | | | 3.28-3.22 (m, 1H), 3.00 (s, 3H), |
| | | | | 2.87-2.77 (m, 1H), 2.46 (s, 3H), |
| | | | | 2.42-2.31 (m, 1H), 2.27-2.18 (m, |
| | | | | 1H), 2.18-1.90 (m, 2H), 1.88- |
| | | | | 1.74 (m, 1H), 1.54-1.30 (m, 3H), |
| | | | | 1.03-0.95 (m, 3H), 0.91-0.77 (m, |
| | | | | 3H). |
| 184 | A | N1-((S)-1-((2S,4R)-4- | 807.00 | 1H NMR (300 MHz, Methanol-d4) δ |
| | | hydroxy-2-((4-(4- | | 8.83 (d, J = 17.2 Hz, 1H), 8.30 (s, |
| | | methylthiazol-5- | | 1H), 7.95-7.84 (m, 1H), 7.47- |
| | | yl)benzyl)carbamoyl)pyrrolidin- | | 7.26 (m, 5H), 7.00-6.94 (m, 2H), |
| | | 1-yl)-3,3-dimethyl-1- | | 4.63-4.52 (m, 4H), 4.42-4.23 (m, |
| | | oxobutan-2-yl)-N5-((S)-3-(2- | | 2H), 3.96-3.66 (m, 2H), 3.15 (dd, |
| | | hydroxyphenyl)-6,7,8,9- | | J = 15.4, 5.3 Hz, 1H), 3.01 (t, J = |
| | | tetrahydro-5H- | | 7.5 Hz, 2H), 2.69 (dd, J = 15.5, 8.2 |
| | | pyridazino[3,4-b]indol-6- | | Hz, 1H), 2.46 (s, 3H), 2.39-2.20 |
| | | yl)glutaramide | | (m, 6H), 2.15-1.93 (m, 4H), 1.04 |
| | | | | (d, J = 15.5 Hz, 9H). |
| 200 | B | (2S,4R)-4-hydroxy-1-((R)-2- | 805.90 | 1H NMR (300 MHz, Methanol-d4) δ |
| | | (3-(2-(((S)-3-(2- | | 8.88 (d, J = 7.2 Hz, 1H), 8.37 (d, J = |
| | | hydroxyphenyl)-6,7,8,9- | | 9 Hz, 1H), 8.04-7.88 (m, 1H), 7.50- |
| | | tetrahydro-5H- | | 7.50 (m, 3H), 7.40-7.25 (m, 2H), |
| | | pyridazino[3,4-b]indol-6- | | 7.03-6.92 (m, 2H), 6.04 (d, J = |
| | | yl)amino)ethoxy)isoxazol-5- | | 16.2 Hz, 1H), 5.11-4.98 (m, 1H), |
| | | yl)-3-methylbutanoyl)-N-((S)- | | 4.81-4.74 (m, 1H), 4.63-4.49 (m, |
| | | 1-(4-(4-methylthiazol-5- | | 1H), 4.49-4.35 (m, 3H), 3.86 (dd, |
| | | yl)phenyl)ethyl)pyrrolidine-2- | | J = 10.8, 4.2 Hz, 1H), 3.79-3.44 |
| | | carboxamide | | (m, 2H), 3.27-3.13 (m, 4H), 3.14- |
| | | | | 2.87 (m, 2H), 2.82-2.66 (m, 1H), |
| | | | | 2.50 (s, 2H), 2.47-2.25 (m, 2H), |
| | | | | 2.25-2.11 (m, 1H), 2.08-1.81 (m, |
| | | | | 2H), 1.57 (dd, J = 17.4, 7.0 Hz, 3H), |
| | | | | 1.07 (d, J = 6.5 Hz, 3H), 0.92 (t, J = |
| | | | | 7.0 Hz, 3H). |
| 160 | B | (2S,4R)-4-hydroxy-1-((R)-2- | 844.50 | 1H NMR (400 MHz, DMSO-d6) δ |
| | | (3-(4-(((R)-3-(2- | | 8.99 (d, J = 4.5 Hz, 1H), 8.54 (d, J = |
| | | hydroxyphenyl)-6,7,8,9- | | 2.8 Hz, 1H), 8.42 (d, J = 7.7 Hz, |
| | | tetrahydro-5H- | | 1H), 8.04 (d, J = 8.0 Hz, 1H), 7.50- |
| | | pyridazino[3,4-b]indol-6- | | 7.42 (m, 2H), 7.41-7.35 (m, 2H), |
| | | yl)amino)piperidin-1- | | 7.32 (t, J = 8.0 Hz, 1H), 7.03-6.96 |
| | | yl)isoxazol-5-yl)-3- | | (m, 2H), 6.23 (s, 1H), 4.91 (q, J = |
| | | methylbutanoyl)-N-((S)-1-(4- | | 7.1 Hz, 1H), 4.39-4.26 (m, 2H), |
| | | (4-methylthiazol-5- | | 3.88-3.70 (m, 4H), 3.65-3.56 (m, |
| | | yl)phenyl)ethyl)pyrrolidine-2- | | 2H), 3.38-3.31 (m, 1H), 3.04 (s, |
| | | carboxamide | | 2H), 2.93 (t, J = 12.6 Hz, 2H), 2.77 |
| | | | | (dd, J = 15.0, 9.4 Hz, 1H), 2.46 (d, |
| | | | | J = 3.0 Hz, 3H), 2.34 (s, 1H), 2.29- |
| | | | | 2.17 (m, 2H), 2.16-2.08 (m, 2H), |
| | | | | 2.07-1.97 (m, 2H), 1.85-1.75 (m, |
| | | | | 1H), 1.70-1.57 (m, 2H), 1.43 (dd, |
| | | | | J = 38.4, 7.0 Hz, 3H), 0.98 (t, J = |
| | | | | 6.4 Hz, 3H), 0.82 (d, J = 6.7 Hz, |
| | | | | 3H). |
| 169 | B | (2S,4R)-4-hydroxy-1-((R)-2- | 844.05 | 1H NMR (400 MHz, DMSO-d6) δ |
| | | (3-(4-(((S)-3-(2- | | 14.45 (s, 1H), 12.21 (s, 1H), 8.99 |
| | | hydroxyphenyl)-6,7,8,9- | | (d, J = 1.8 Hz, 1H), 8.54-8.36 (m, |
| | | tetrahydro-5H- | | 2H), 8.14-8.06 (m, 1H), 7.48- |
| | | pyridazino[3,4-b]indol-6- | | 7.42 (m, 2H), 7.37 (d, J = 8.1 Hz, |
| | | yl)amino)piperidin-1- | | 2H), 7.31-7.25 (m, 1H), 6.94 (t, |
| | | yl)isoxazol-5-yl)-3- | | J = 8.1 Hz, 2H), 6.14 (s, 1H), 5.11 (d, |
| | | methylbutanoyl)-N-((S)-1-(4- | | J = 3.7 Hz, 1H), 4.92 (q, J = 7.1 Hz, |
| | | (4-methylthiazol-5- | | 1H), 4.38 (d, J = 7.9 Hz, 1H), 4.27 |
| | | yl)phenyl)ethyl)pyrrolidine-2- | | (d, J = 7.2 Hz, 1H), 3.72 (dd, J = |
| | | carboxamide | | 10.5, 4.5 Hz, 1H), 3.60 (dd, J = |
| | | | | 22.4, 11.3 Hz, 3H), 3.43 (d, J = 10.8 |
| | | | | Hz, 1H), 3.23-3.16 (m, 1H), 3.10 |
| | | | | (d, J = 15.4 Hz, 1H), 2.97-2.78 (m, |
| | | | | 5H), 2.46 (s, 3H), 2.42-2.38 (m, |
| | | | | 1H), 2.27-2.16 (m, 2H), 2.11- |
| | | | | 1.99 (m, 2H), 1.95-1.85 (m, 2H), |
| | | | | 1.82-1.70 (m, 2H), 1.38 (d, J = 7.0 |
| | | | | Hz, 3H), 1.34-1.25 (m, 2H), 0.95 |
| | | | | (d, J = 6.5 Hz, 3H), 0.82 (dd, J = |
| | | | | 15.2, 6.6 Hz, 3H). |
| 107 | B | (2S,4R)-4-hydroxy-1-((R)-2- | 872.40 | 1H NMR (300 MHz, DMSO-d6) δ |
| | | (3-(4-((((S)-3-(2- | | 14.47 (s, 1H), 12.22 (s, 1H), 8.99 (s, |
| | | hydroxyphenyl)-6,7,8,9- | | 1H), 8.56 (s, 1H), 8.41 (d, J = 7.7 |
| | | tetrahydro-5H- | | Hz, 1H), 8.11 (d, J = 7.9 Hz, 1H), |
| | | pyridazino[3,4-b]indol-6- | | 7.48-7.33 (m, 4H), 7.33-7.24 (m, |
| | | yl)(methyl)amino)methyl)piperidin- | | 1H), 7.00-6.91 (m, 2H), 6.13 (s, |
| | | 1-yl)isoxazol-5-yl)-3- | | 1H), 5.11 (d, J = 3.6 Hz, 1H), 4.92 |
| | | methylbutanoyl)-N-((S)-1-(4- | | (t, J = 7.1 Hz, 1H), 4.45-4.18 (m, |
| | | (4-methylthiazol-5- | | 2H), 3.79-3.48 (m, 5H), 3.48- |
| | | yl)phenyl)ethyl)pyrrolidine-2- | | 3.37 (m, 2H), 3.04-2.85 (m, 4H), |
| | | carboxamide | | 2.85-2.69 (m, 2H), 2.69-2.59 (m, |
| | | | | 1H), 2.46 (s, 3H), 2.44-2.36 (m, |
| | | | | 2H), 2.33 (s, 3H), 2.30-2.15 (m, |
| | | | | 1H), 2.14-1.93 (m, 2H), 1.91- |
| | | | | 1.56 (m, 5H), 1.38 (d, J = 7.0 Hz, |
| | | | | 3H), 0.95 (d, J = 6.4 Hz, 3H), 0.80 |
| | | | | (d, J = 6.7 Hz, 3H). |
| 111 | B | (2S,4R)-4-hydroxy-1-((R)-2- | 858.50 | 1H NMR (300 MHz, DMSO-d6) δ |
| | | (3-(4-(((S)-3-(2- | | 14.46 (s, 1H), 12.22 (s, 1H), 8.99 (s, |
| | | hydroxyphenyl)-6,7,8,9- | | 1H), 8.55 (s, 1H), 8.41 (d, J = 7.7 |
| | | tetrahydro-5H- | | Hz, 1H), 8.09 (d, J = 7.9 Hz, 1H), |
| | | pyridazino[3,4-b]indol-6- | | 7.50-7.41 (m, 2H), 7.40-7.35 (m, |
| | | yl)(methyl)amino)piperidin-1- | | 2H), 7.33-7.23 (m, 1H), 7.00- |
| | | yl)isoxazol-5-yl)-3- | | 6.91 (m, 2H), 6.15 (s, 1H), 5.11 (d, |
| | | methylbutanoyl)-N-((S)-1-(4- | | J = 3.6 Hz, 1H), 4.92 (t, J = 7.2 Hz, |
| | | (4-methylthiazol-5- | | 1H), 4.37 (t, J = 7.8 Hz, 1H), 4.29 |
| | | yl)phenyl)ethyl)pyrrolidine-2- | | (s, 1H), 3.78-3.63 (m, 3H), 3.63- |
| | | carboxamide | | 3.53 (m, 1H), 3.48-3.39 (m, 1H), |
| | | | | 3.16 (s, 1H), 3.05-2.90 (m, 3H), |
| | | | | 2.90-2.65 (m, 3H), 2.46 (s, 3H), |
| | | | | 2.38-2.22 (m, 3H), 2.21-2.12 (m, |
| | | | | 1H), 2.11-1.96 (m, 2H), 1.96- |
| | | | | 1.71 (m, 4H), 1.69-1.49 (m, 3H), |
| | | | | 1.38 (d, J = 6.9 Hz, 3H), 0.95 (d, J = |
| | | | | 6.3 Hz, 3H), 0.80 (d, J = 6.7 Hz, |
| | | | | 3H). |
| 156 | B | (2S,4R)-4-hydroxy-1-((R)-2- | 819.20 | 1H NMR (400 MHz, DMSO-d6) δ |
| | | (3-(2-(((R)-3-(2- | | 14.45 (s, 1H), 12.21 (s, 1H), 8.98 |
| | | hydroxyphenyl)-6,7,8,9- | | (d, J = 1.3 Hz, 1H), 8.53 (d, J = 2.6 |
| | | tetrahydro-5H- | | Hz, 1H), 8.41 (d, J = 7.7 Hz, 1H), |
| | | pyridazino[3,4-b]indol-6- | | 8.08 (td, J = 7.1, 6.1, 1.5 Hz, 1H), |
| | | yl)(methyl)amino)ethoxy)isoxazol- | | 7.44 (d, J = 8.3 Hz, 2H), 7.39-7.32 |
| | | 5-yl)-3-methylbutanoyl)- | | (m, 2H), 7.30-7.24 (m, 1H), 6.94 |
| | | N-((S)-1-(4-(4-methylthiazol- | | (t, J = 7.5 Hz, 2H), 6.11 (s, 1H), |
| | | 5-yl)phenyl)ethyl)pyrrolidine- | | 5.10 (d, J = 3.7 Hz, 1H), 4.95-4.87 |
| | | 2-carboxamide | | (m, 1H), 4.41-4.20 (m, 4H), 3.70 |
| | | | | (dd, J = 10.5, 4.4 Hz, 1H), 3.65 (d, |
| | | | | J = 9.8 Hz, 1H), 3.49-3.41 (m, 1H), |
| | | | | 3.06-2.82 (m, 6H), 2.69-2.56 (m, |
| | | | | 1H), 2.45 (s, 3H), 2.39 (s, 3H), 2.29- |
| | | | | 2.17 (m, 1H), 2.13-1.97 (m, 2H), |
| | | | | 1.86-1.73 (m, 2H), 1.41 (dd, J = |
| | | | | 26.1, 7.0 Hz, 3H), 0.96 (d, J = 6.3 |
| | | | | Hz, 3H), 0.81 (dd, J = 14.1, 6.6 Hz, |
| | | | | 3H). |
| 165 | B | (2S,4R)-4-hydroxy-1-((R)-2- | 819.50 | 1H NMR (400 MHz, DMSO-d6) δ |
| | | (3-(2-(((S)-3-(2- | | 14.45 (s, 1H), 12.22 (s, 1H), 8.98 (s, |
| | | hydroxyphenyl)-6,7,8,9- | | 1H), 8.54 (d, J = 2.8 Hz, 1H), 8.41 |
| | | tetrahydro-5H- | | (d, J = 7.7 Hz, 1H), 8.09 (d, J = 7.7 |
| | | pyridazino[3,4-b]indol-6- | | Hz, 1H), 7.44 (d, J = 7.9 Hz, 2H), |
| | | yl)(methyl)amino)ethoxy)isoxazol- | | 7.36 (d, J = 8.0 Hz, 2H), 7.28 (t, J = |
| | | 5-yl)-3-methylbutanoyl)- | | 7.7 Hz, 1H), 6.95 (d, J = 7.8 Hz, |
| | | N-((S)-1-(4-(4-methylthiazol- | | 2H), 6.11 (s, 1H), 5.10 (d, J = 3.6 |
| | | 5-yl)phenyl)ethyl)pyrrolidine- | | Hz, 1H), 4.91 (t, J = 7.2 Hz, 1H), |
| | | 2-carboxamide | | 4.37 (t, J = 7.9 Hz, 1H), 4.25 (t, J = |
| | | | | 6.0 Hz, 3H), 3.74-3.61 (m, 2H), |
| | | | | 3.45 (d, J = 10.3 Hz, 1H), 3.06- |
| | | | | 2.82 (m, 6H), 2.64-2.58 (m, 1H), |
| | | | | 2.47-2.44 (m, 3H), 2.39 (s, 3H), |
| | | | | 2.29-2.18 (m, 1H), 2.13-1.97 (m, |
| | | | | 2H), 1.87-1.74 (m, 2H), 1.41 (dd, |
| | | | | J = 26.4, 7.0 Hz, 3H), 0.95 (d, J = |
| | | | | 6.4 Hz, 3H), 0.81 (dd, J = 14.2, 6.6 |
| | | | | Hz, 3H). |
| 106 | A | (2S,4R)-4-hydroxy-1-((R)-2- | 833.15 | 1H NMR (300 MHz, Methanol-d4) δ |
| | | (3-(2-(((S)-3-(2- | | 8.88 (s, 1H), 8.32 (s, 1H), 8.00- |
| | | hydroxyphenyl)-6,7,8,9- | | 7.92 (m, 1H), 7.50-7.23 (m, 5H), |
| | | tetrahydro-5H- | | 7.02-6.93 (m, 2H), 6.15-6.05 (m, |
| | | pyridazino[3,4-b]indol-6- | | 1H), 5.15 (s, 1H), 5.10-4.98 (m, |
| | | yl)(methyl)amino)-2- | | 2H), 4.84-4.72 (m, 1H), 4.58- |
| | | oxoethoxy)isoxazol-5-yl)-3- | | 4.49 (m, 1H), 4.49-4.45 (m, 1H), |
| | | methylbutanoyl)-N-((S)-1-(4- | | 3.91-3.77 (m, 1H), 3.75-3.55 (m, |
| | | (4-methylthiazol-5- | | 2H), 3.21-2.99 (m, 6H), 2.96- |
| | | yl)phenyl)ethyl)pyrrolidine-2- | | 2.86 (m, 1H), 2.50 (s, 3H), 2.47- |
| | | carboxamide | | 2.24 (m, 2H), 2.23-2.11 (m, 1H), |
| | | | | 2.11-1.84 (m, 2H), 1.52 (d, 3H), |
| | | | | 1.08 (d, J = 6.6 Hz, 3H), 0.92 (d, J = |
| | | | | 6.8 Hz, 3H). |
| 69 | A | (2S,4R)-4-hydroxy-1-((R)-2- | 845.50 | 1H NMR (400 MHz, Methanol-d4) δ |
| | | (3-(1-((1R,3s)-3′-(2- | | 8.86 (d, J = 7.2 Hz, 1H), 7.90 (s, |
| | | hydroxyphenyl)-5′,7′- | | 1H), 7.75 (d, J = 7.6 Hz, 1H), 7.47- |
| | | dihydrospiro[cyclobutane- | | 7.33 (m, 4H), 7.28-7.20 (m, 1H), |
| | | 1,6′-pyrrolo[2,3- | | 6.91 (dd, J = 8.2, 6.6 Hz, 2H), 6.32 |
| | | c]pyridazine]-3- | | (s, 1H), 4.59-4.43 (m, 4H), 3.90- |
| | | carbonyl)piperidin-4- | | 3.75 (m, 3H), 3.65-3.57 (m, 1H), |
| | | yl)isoxazol-5-yl)-3- | | 3.48-3.38 (m, 2H), 3.34 (s, 3H), |
| | | methylbutanoyl)-N-((S)-1-(4- | | 3.13-3.03 (m, 1H), 2.94-2.83 (m, |
| | | (4-methylthiazol-5- | | 1H), 2.67-2.58 (m, 4H), 2.47 (s, |
| | | yl)phenyl)ethyl)pyrrolidine-2- | | 3H), 2.41-2.31 (m, 1H), 2.22- |
| | | carboxamide | | 2.12 (m, 1H), 2.03-1.89 (m, 3H), |
| | | | | 1.64-1.58 (m, 3H), 1.51 (dd, J = |
| | | | | 7.0, 2.9 Hz, 3H), 1.06 (d, J = 6.6 |
| | | | | Hz, 3H), 0.86 (dd, J = 10.1, 6.6 Hz, |
| | | | | 3H). |
| 70 | A | (1R,3s)-N-(2-((5-((R)-1- | 821.45 | 1H NMR (400 MHz, Methanol-d4) δ |
| | | ((2S,4R)-4-hydroxy-2-(((S)- | | 8.86 (d, J = 10.6 Hz, 1H), 7.92 (d, |
| | | 1-(4-(4-methylthiazol-5- | | J = 1.6 Hz, 1H), 7.77 (dd, J = 8.3, 1.6 |
| | | yl)phenyl)ethyl)carbamoyl)pyrrolidin- | | Hz, 1H), 7.47-7.32 (m, 4H), 7.24 |
| | | 1-yl)-3-methyl-1- | | (td, J = 7.7, 1.5 Hz, 1H), 6.91 (ddt, |
| | | oxobutan-2-yl)isoxazol-3- | | J = 7.9, 4.0, 2.4 Hz, 2H), 6.01 (s, 1H), |
| | | yl)oxy)ethyl)-3′-(2- | | 5.01 (d, J = 7.0 Hz, 1H), 4.49-4.30 |
| | | hydroxyphenyl)-5′,7′- | | (m, 2H), 4.29 (t, J = 5.3 Hz, 2H), |
| | | dihydrospiro[cyclobutane- | | 3.81 (dd, J = 10.9, 4.2 Hz, 1H), 3.75- |
| | | 1,6′-pyrrolo[2,3- | | 3.55 (m, 4H), 3.36-3.34 (m, 1H), |
| | | c]pyridazine]-3-carboxamide | | 3.32-3.29 (m, 3H), 3.13-3.06 (m, |
| | | | | 1H), 2.66-2.56 (m, 2H), 2.56- |
| | | | | 2.46 (m, 5H), 2.36 (dq, J = 16.4, 6.5 |
| | | | | Hz, 1H), 2.16 (dd, J = 13.2, 8.0 Hz, |
| | | | | 1H), 2.01-1.87 (m, 1H), 1.58- |
| | | | | 1.48 (m, 3H), 1.03 (d, J = 6.5 Hz, |
| | | | | 3H), 0.87 (dd, J = 10.8, 6.7 Hz, 3H). |
| 71 | A | (1S,3r)-N-(2-((5-((R)-1- | 821.20 | 1H NMR (400 MHz, Methanol-d4) δ |
| | | ((2S,4R)-4-hydroxy-2-(((S)- | | 8.87 (s, 1H), 7.91 (d, J = 1.7 Hz, |
| | | 1-(4-(4-methylthiazol-5- | | 1H), 7.78-7.67 (m, 1H), 7.47- |
| | | yl)phenyl)ethyl)carbamoyl)pyrrolidin- | | 7.32 (m, 4H), 7.24 (td, J = 7.6, 1.6 |
| | | 1-yl)-3-methyl-1- | | Hz, 1H), 6.95-6.84 (m, 2H), 6.01 |
| | | oxobutan-2-yl)isoxazol-3- | | (s, 1H), 5.03 (q, J = 7.1 Hz, 1H), |
| | | yl)oxy)ethyl)-3′-(2- | | 4.54-4.36 (m, 2H), 4.27 (t, J = 5.3 |
| | | hydroxyphenyl)-5′,7′- | | Hz, 2H), 3.86-3.46 (m, 5H), 3.40- |
| | | dihydrospiro[cyclobutane- | | 3.32 (m, 2H), 2.92-2.79 (m, 1H), |
| | | 1,6′-pyrrolo[2,3- | | 2.56 (m, J = 12.5, 9.3 Hz, 2H), 2.49- |
| | | c]pyridazine]-3-carboxamide | | 2.34 (m, 6H), 2.16 (m, J = 13.4, |
| | | | | 7.9 Hz, 1H), 1.94 (m, J = 13.2, 8.8, |
| | | | | 4.5 Hz, 1H), 1.55 (m, J = 27.0, 7.0 |
| | | | | Hz, 3H), 1.05 (d, J = 6.5 Hz, 3H), |
| | | | | 0.89 (t, J = 7.0 Hz, 3H). |
| 72 | A | (2S,4R)-4-hydroxy-1-((R)-2- | 858.20 | 1H NMR (300 MHz, Methanol-d4) δ |
| | | (3-((1S,4S)-5-((1S,3s)-3′-(2- | | 8.89 (s, 1H), 7.91 (s, 1H), 7.81- |
| | | hydroxyphenyl)-5′,7′- | | 7.73 (m, 1H), 7.50-7.40 (m, 3H), |
| | | dihydrospiro[cyclobutane- | | 7.39-7.16 (m, 2H), 6.97-6.84 (m, |
| | | 1,6′-pyrrolo[2,3- | | 2H), 6.12-5.95 (m, 1H), 5.10- |
| | | c]pyridazine]-3-carbonyl)- | | 4.99 (m, 1H), 4.86-4.84 (m, |
| | | 2,5- | | 1H),4.66-4.50 (m, 1H), 4.46 (d, |
| | | diazabicyclo[2.2.1]heptan-2- | | J = 13.1 Hz, 2H), 3.92-3.71 (m, 1H), |
| | | yl)isoxazol-5-yl)-3- | | 3.71-3.39 (m, 6H), 3.30-3.22 (m, |
| | | methylbutanoyl)-N-((S)-1-(4- | | 1H), 3.21-3.07 (m, 1H), 2.77- |
| | | (4-methylthiazol-5- | | 2.53 (m, 3H), 2.50 (d, J = 1.5 Hz, |
| | | yl)phenyl)ethyl)pyrrolidine-2- | | 3H), 2.49-2.15 (m, 3H), 2.15- |
| | | carboxamide | | 1.82 (m, 4H), 1.58-1.50 (m, 3H), |
| | | | | 1.11-0.99 (m, 3H), 0.95-0.83 (m, |
| | | | | 3H). |
| 75 | A | (2S,4R)-4-hydroxy-1-((R)-2- | 845.45 | 1H NMR (400 MHz, DMSO-d6) δ |
| | | (3-(1-((1S,3r)-3′-(2- | | 14.23 (s, 1H), 8.98 (s, 1H), 8.40 (d, |
| | | hydroxyphenyl)-5′,7′- | | J = 7.8 Hz, 1H), 8.13-7.95 (m, |
| | | dihydrospiro[cyclobutane- | | 2H), 7.90-7.83 (m, 1H), 7.49- |
| | | 1,6′-pyrrolo[2,3- | | 7.40 (m, 2H), 7.36 (d, J = 8.2 Hz, |
| | | c]pyridazine]-3- | | 2H), 7.24 (td, J = 7.6, 1.6 Hz, 1H), |
| | | carbonyl)piperidin-4- | | 6.93-6.85 (m, 2H), 6.34 (s, 1H), |
| | | yl)isoxazol-5-yl)-3- | | 5.08 (d, J = 3.7 Hz, 1H), 4.91 (t, J = |
| | | methylbutanoyl)-N-((S)-1-(4- | | 7.2 Hz, 1H), 4.40-4.32 (m, 2H), |
| | | (4-methylthiazol-5- | | 4.28 (s, 1H), 3.88 (d, J = 13.1 Hz, |
| | | yl)phenyl)ethyl)pyrrolidine-2- | | 1H), 3.80-3.52 (m, 2H), 3.41- |
| | | carboxamide | | 3.52 (m, 1H), 3.42-3.38 (m, 2H), |
| | | | | 3.18-3.03 (m, 2H), 3.03-2.90 (m, |
| | | | | 1H), 2.74 (t, J = 12.2 Hz, 1H), 2.48- |
| | | | | 2.45 (m, 4H), 2.38-2.12 (m, 4H), |
| | | | | 2.10-1.97 (m, 1H), 1.90 (s, 2H), |
| | | | | 1.84-1.72 (m, 1H), 1.60-1.32 (m, |
| | | | | 5H), 0.97 (d, J = 6.4 Hz, 3H), 0.78 |
| | | | | (dd, J = 11.9, 6.7 Hz, 3H). |
| 76 | A | (2S,4R)-4-hydroxy-1-((R)-2- | 858.15 | 1H NMR (400 MHz, Methanol-d4) δ |
| | | (3-((1R,4R)-5-((1S,3r)-3′-(2- | | 8.87 (d, J = 1.2 Hz, 1H), 7.92 (dd, |
| | | hydroxyphenyl)-5′,7′- | | J = 16.8, 1.7 Hz, 1H), 7.75 (td, J = |
| | | dihydrospiro[cyclobutane- | | 8.1, 1.6 Hz, 1H), 7.47-7.32 (m, |
| | | 1,6′-pyrrolo[2,3- | | 4H), 7.31-7.17 (m, 1H), 6.99- |
| | | c]pyridazine]-3-carbonyl)-2,5- | | 6.84 (m, 2H), 6.10-5.88 (m, 1H), |
| | | diazabicyclo[2.2.1]heptan-2- | | 5.03 (dd, J = 7.0, 3.4 Hz, 1H), 4.73 |
| | | yl)isoxazol-5-yl)-3- | | (s, 1H), 4.59 (s, 1H), 4.54-4.33 |
| | | methylbutanoyl)-N-((S)-1-(4- | | (m, 3H), 3.82 (ddd, J = 14.9, 10.9, |
| | | (4-methylthiazol-5- | | 4.2 Hz, 1H), 3.67-3.54 (m, 3H), |
| | | yl)phenyl)ethyl)pyrrolidine-2- | | 3.45 (t, J = 2.4 Hz, 2H), 3.37 (t, J = |
| | | carboxamide | | 1.6 Hz, 1H), 3.20-3.10 (m, 1H), |
| | | | | 3.00-2.86 (m, 1H), 2.71-2.55 (m, |
| | | | | 2H), 2.47 (d, J = 2.0 Hz, 3H), 2.40- |
| | | | | 2.24 (m, 3H), 2.12-1.82 (m, 4H), |
| | | | | 1.62-1.45 (m, 3H), 1.11-0.98 (m, |
| | | | | 3H), 0.95-0.82 (m, 3H). |
| 77 | A | (2S,4R)-4-hydroxy-1-((R)-2- | 846.15 | 1H NMR (400 MHz, DMSO-d6) δ |
| | | (3-(4-((1S,3r)-3′-(2- | | 14.23 (s, 1H), 8.98 (s, 1H), 8.40 (d, |
| | | hydroxyphenyl)-5′,7′- | | J = 7.7 Hz, 1H), 8.05 (d, J = 14.0 |
| | | dihydrospiro[cyclobutane- | | Hz, 2H), 7.90-7.84 (m, 1H), 7.49- |
| | | 1,6′-pyrrolo[2,3- | | 7.40 (m, 2H), 7.40-7.33 (m, 2H), |
| | | c]pyridazine]-3- | | 7.29-7.20 (m, 1H), 6.94-6.85 (m, |
| | | carbonyl)piperazin-1- | | 2H), 6.20 (s, 1H), 5.10 (d, J = 3.7 |
| | | yl)isoxazol-5-yl)-3- | | Hz, 1H), 4.91 (t, J = 7.2 Hz, 1H), |
| | | methylbutanoyl)-N-((S)-1-(4- | | 4.36 (t, J = 7.9 Hz, 1H), 4.28 (s, |
| | | (4-methylthiazol-5- | | 1H), 3.76-3.67 (m, 1H), 3.63- |
| | | yl)phenyl)ethyl)pyrrolidine-2- | | 3.50 (m, 5H), 3.45-3.39 (m, 2H), |
| | | carboxamide | | 3.30-3.29 (m, 1H), 3.19-3.13 (m, |
| | | | | 5H), 2.46 (s, 4H), 2.38-2.31 (m, |
| | | | | 3H), 2.28-2.17 (m, 1H), 2.06- |
| | | | | 1.97 (m, 1H), 1.84-1.73 (m, 1H), |
| | | | | 1.48-1.35 (m, 3H), 1.00-0.92 (m, |
| | | | | 3H), 0.86-0.76 (m, 3H). |
| 78 | A | (2S,4R)-4-hydroxy-1-((R)-2- | 858.15 | 1H NMR (300 MHz, DMSO-d6) δ |
| | | (3-((1S,4S)-5-((1R,3r)-3′-(2- | | 14.23 (s, 1H), 8.99 (s, 1H), 8.59- |
| | | hydroxyphenyl)-5′,7′- | | 8.20 (m, 1H), 8.19-7.69 (m, 3H), |
| | | dihydrospiro[cyclobutane- | | 7.52-7.32 (m, 4H), 7.32-7.19 (m, |
| | | 1,6′-pyrrolo[2,3- | | 1H), 7.00-6.55 (m, 2H), 6.16- |
| | | c]pyridazine]-3-carbonyl)-2,5- | | 5.90 (m, 1H), 5.17-5.04 (m, 1H), |
| | | diazabicyclo[2.2.1]heptan-2- | | 4.92 (t, J = 7.2 Hz, 1H), 4.69 (d, J = |
| | | yl)isoxazol-5-yl)-3- | | 17.7 Hz, 1H), 4.48-4.06 (m, 3H), |
| | | methylbutanoyl)-N-((S)-1-(4- | | 3.79-3.65 (m, 1H), 3.64-3.40 (m, |
| | | (4-methylthiazol-5- | | 1H), 3.40-3.36 (m, 4H), 3.23- |
| | | yl)phenyl)ethyl)pyrrolidine-2- | | 3.02 (m, 3H), 2.95-2.64 (m, 1H), |
| | | carboxamide | | 2.50-2.40 (m, 5H), 2.35-2.10 (m, |
| | | | | 3H), 2.09-1.71 (m, 4H), 1.38 (d, |
| | | | | J = 7.2 Hz, 3H), 1.01-0.90 (m, 3H), |
| | | | | 0.85-0.75 (m, 3H). |
| 79 | A | (2S,4R)-4-hydroxy-1-((R)-2- | 858.50 | 1H NMR (400 MHz, Methanol-d4) δ |
| | | (3-((1R,4R)-5-((1R,3s)-3′-(2- | | 8.87 (d, J = 4.0 Hz, 1H), 7.88 (d, J = |
| | | hydroxyphenyl)-5′,7′- | | 16.2 Hz, 1H), 7.74 (t, J = 9.1 Hz, |
| | | dihydrospiro[cyclobutane- | | 1H), 7.47-7.29 (m, 4H), 7.23 (td, J = |
| | | 1,6′-pyrrolo[2,3- | | 7.8, 1.4 Hz, 1H), 6.90 (t, J = 7.3 |
| | | c]pyridazine]-3-carbonyl)-2,5- | | Hz, 2H), 5.99 (dd, J = 29.8, 7.6 Hz, |
| | | diazabicyclo[2.2.1]heptan-2- | | 1H), 4.59 (s, 1H), 4.49 (q, J = 8.6 |
| | | yl)isoxazol-5-yl)-3- | | Hz, 1H), 4.42 (s, 2H), 3.83 (td, J = |
| | | methylbutanoyl)-N-((S)-1-(4- | | 11.1, 4.1 Hz, 1H), 3.68-3.49 (m, |
| | | (4-methylthiazol-5- | | 4H), 3.46 (d, J = 5.9 Hz, 2H), 3.22 |
| | | yl)phenyl)ethyl)pyrrolidine-2- | | (d, J = 9.5 Hz, 1H), 2.70-2.53 (m, |
| | | carboxamide | | 3H), 2.50-2.42 (m, 3H), 2.34 (ddd, |
| | | | | J = 25.1, 10.2, 6.2 Hz, 2H), 2.22- |
| | | | | 2.12 (m, 1H), 2.07-1.88 (m, 3H), |
| | | | | 1.61-1.45 (m, 3H), 1.10-0.98 (m, |
| | | | | 3H), 0.92-0.83 (m, 3H). |
| 80 | A | (2S,4R)-4-hydroxy-1-((R)-2- | 846.20 | 1H NMR (400 MHz, DMSO-d6) δ |
| | | (3-(4-((1R,3s)-3′-(2- | | 14.22 (s, 1H), 8.98 (s, 1H), 8.40 (d, |
| | | hydroxyphenyl)-5′,7′- | | J = 7.7 Hz, 1H), 8.19 (s, 1H), 8.01 |
| | | dihydrospiro[cyclobutane- | | (s, 1H), 7.81 (d, J = 7.8 Hz, 1H), |
| | | 1,6′-pyrrolo[2,3- | | 7.49-7.40 (m, 2H), 7.40-7.33 (m, |
| | | c]pyridazine]-3- | | 2H), 7.28-7.20 (m, 1H), 6.93- |
| | | carbonyl)piperazin-1- | | 6.85 (m, 2H), 6.20 (s, 1H), 5.10 (d, |
| | | yl)isoxazol-5-yl)-3- | | J = 3.6 Hz, 1H), 4.95-4.87 (m, |
| | | methylbutanoyl)-N-((S)-1-(4- | | 1H), 4.40-4.31 (m, 1H), 4.28 (s, |
| | | (4-methylthiazol-5- | | 1H), 3.74-3.68 (m, 1H), 3.63- |
| | | yl)phenyl)ethyl)pyrrolidine-2- | | 3.56 (m, 3H), 3.45-3.41 (m, 2H), |
| | | carboxamide | | 3.30 (s, 2H), 3.24-3.20 (m, 3H), |
| | | | | 3.20-3.16 (m, 3H), 2.45 (s, 4H), |
| | | | | 2.26-2.21 (m, 3H), 2.03-1.99 (m, |
| | | | | 2H), 1.84-1.73 (m, 1H), 1.38 (d, |
| | | | | J = 7.0 Hz, 3H), 0.98-0.92 (m, 3H), |
| | | | | 0.88-0.76 (m, 3H). |
| 84 | B | (2S,4R)-4-hydroxy-1-((R)-2- | 793.34 | 1H NMR (300 MHz, Methanol-d4) δ |
| | | (3-(3-(3′-(2-hydroxyphenyl)- | | 8.82 (s, 1H), 8.20 (s, FA, 1H), 8.01 |
| | | 5′,7′-dihydrospiro[azetidine- | | (s, 1H), 7.81-7.74 (m, 1H), 7.51- |
| | | 3,6′-pyrrolo[2,3-c]pyridazin]- | | 7.38 (m, 4H), 7.33-7.23 (m, 1H), |
| | | 1-yl)propoxy)isoxazol-5-yl)- | | 6.99-6.88 (m, 2H), 5.96-5.79 (m, |
| | | 3-methylbutanoyl)-N-((S)-1- | | 1H), 5.11-4.96 (m, 1H), 4.57 (t, |
| | | (4-(4-methylthiazol-5- | | J = 8.3 Hz, 1H), 4.21-4.00 (m, 4H), |
| | | yl)phenyl)ethyl)pyrrolidine-2- | | 3.99-3.78 (m, 3H), 3.76-3.60 (m, |
| | | carboxamide | | 4H), 3.57-3.48 (m, 1H), 3.47- |
| | | | | 3.36 (m, 1H), 3.11-2.99 (m, 2H), |
| | | | | 2.55-2.40 (m, 4H), 2.32-2.17 (m, |
| | | | | 1H), 2.07-1.90 (m, 3H), 1.57 (d, J = |
| | | | | 7.0 Hz, 3H), 1.11 (d, J = 6.5 Hz, |
| | | | | 3H), 1.02 (d, J = 6.7 Hz, 3H). |
| 87 | B | (2S,4R)-4-hydroxy-1-((R)-2- | 792.20 | 1H NMR (300 MHz, DMSO-d6) δ |
| | | (3-((2-(3′-(2-hydroxyphenyl)- | | 14.18 (s, 1H), 8.99 (d, J = 1.9 Hz, |
| | | 5′,7′-dihydrospiro[azetidine- | | 1H), 8.42 (d, J = 7.6 Hz, 1H), 8.17 |
| | | 3,6′-pyrrolo[2,3-c]pyridazin]-1- | | (s, 1H), 8.04 (d, J = 10.2 Hz, 1H), |
| | | yl)ethyl)(methyl)amino)isoxazol- | | 7.83 (td, J = 8.7, 1.6 Hz, 1H), 7.50- |
| | | 5-yl)-3-methylbutanoyl)- | | 7.40 (m, 2H), 7.40-7.31 (m, 2H), |
| | | N-((S)-1-(4-(4-methylthiazol- | | 7.25 (td, J = 7.6, 1.6 Hz, 1H), 7.00- |
| | | 5-yl)phenyl)ethyl)pyrrolidine- | | 6.79 (m, 2H), 5.98 (d, J = 35.2 Hz, |
| | | 2-carboxamide | | 1H), 5.25-4.98 (m, 1H), 4.97- |
| | | | | 4.63 (m, 1H), 4.38 (t, J = 7.8 Hz, |
| | | | | 1H), 4.30 (s, 1H), 3.73 (dd, J = |
| | | | | 10.5, 4.4 Hz, 1H), 3.56 (d, J = 9.8 |
| | | | | Hz, 1H), 3.53-3.40 (m, 5H), 3.16 |
| | | | | (t, J = 5.8 Hz, 4H), 2.86 (s, 3H), |
| | | | | 2.59 (t, J = 6.5 Hz, 2H), 2.45 (d, J = |
| | | | | 3.5 Hz, 3H), 2.33-2.12 (m, 1H), |
| | | | | 2.10-1.97 (m, 1H), 1.80 (ddd, J = |
| | | | | 12.5, 7.9, 4.7 Hz, 1H), 1.42 (dd, J = |
| | | | | 25.8, 7.0 Hz, 3H), 0.96 (d, J = 6.3 |
| | | | | Hz, 3H), 0.83 (dd, J = 10.3, 6.6 Hz, |
| | | | | 3H). |
| 95 | A | (2S,4R)-4-hydroxy-1-((R)-2- | 793.40 | 1H NMR (300 MHz, DMSO-d6) δ |
| | | (3-(2-(3′-(2-hydroxyphenyl)- | | 14.10 (s, 1H), 8.99 (s, 1H), 8.48- |
| | | 5′,7′-dihydrospiro[azetidine- | | 8.40 (m, 2H), 8.11 (s, 1H), 7.86 (d, |
| | | 3,6′-pyrrolo[2,3-c]pyridazin]- | | J = 8.0 Hz, 1H), 7.48-7.32 (m, |
| | | 1-yl)-2-oxoethoxy)isoxazol- | | 4H), 7.26 (t, J = 7.7 Hz, 1H), 6.91 |
| | | 5-yl)-3-methylbutanoyl)-N- | | (d, J = 7.8 Hz, 2H), 6.17 (s, 1H), |
| | | ((S)-1-(4-(4-methylthiazol-5- | | 5.16-4.84 (m, 2H), 4.75 (s, 2H), |
| | | yl)phenyl)ethyl)pyrrolidine-2- | | 4.45-4.23 (m, 4H), 4.21-4.06 (m, |
| | | carboxamide | | 2H), 3.74-3.66 (m, 2H), 3.58- |
| | | | | 3.52 (m, 2H), 3.51-3.42 (m, 1H), |
| | | | | 2.46 (s, 3H), 2.33-2.11 (m, 1H), |
| | | | | 2.09-2.00 (m, 1H), 1.82-1.72 (m, |
| | | | | 1H), 1.42 (d, J = 26.0, 6.9 Hz, 3H), |
| | | | | 0.97 (d, J = 6.5 Hz, 3H), 0.81 (d, J = |
| | | | | 6.8 Hz, 3H). |
| 96 | B | (2S,4R)-4-hydroxy-1-((R)-2- | 832.25 | 1H NMR (300 MHz, DMSO-d6) δ |
| | | (3-(4-((3′-(2-hydroxyphenyl)- | | 14.36 (s, 1H), 9.07 (s, 1H), 8.48 (d, |
| | | 5′,7′-dihydrospiro[azetidine- | | J = 7.6 Hz, 1H), 8.26 (s, 1H), 8.12 |
| | | 3,6′-pyrrolo[2,3-c]pyridazin]- | | (s, 1H), 7.92 (d, J = 7.9 Hz, 1H), |
| | | 1-yl)methyl)piperidin-1- | | 7.58-7.40 (m, 4H), 7.37-7.26 (m, |
| | | yl)isoxazol-5-yl)-3- | | 1H), 6.98 (d, J = 7.8 Hz, 2H), 6.19 |
| | | methylbutanoyl)-N-((S)-1-(4- | | (s, 1H), 5.25-4.92 (m, 2H), 4.50- |
| | | (4-methylthiazol-5- | | 4.28 (m, 2H), 3.90-3.61 (m, 4H), |
| | | yl)phenyl)ethyl)pyrrolidine-2- | | 3.51 (d, J = 10.0 Hz, 4H), 3.18 (d, |
| | | carboxamide | | J = 6.8 Hz, 2H), 2.89-2.73 (m, 2H), |
| | | | | 2.53 (s, 3H), 2.41-2.20 (m, 3H), |
| | | | | 2.20-2.02 (m, 1H), 1.80 (d, J = |
| | | | | 13.7 Hz, 3H), 1.50-1.38 (m, 4H), |
| | | | | 1.30-1.12 (m, 3H), 1.03 (d, J = 6.5 |
| | | | | Hz, 3H), 0.86 (d, J = 6.7 Hz, 3H). |
| 97 | A | (2S,4R)-4-hydroxy-1-((R)-2- | 847.10 | 1H NMR (300 MHz, DMSO-d6) δ |
| | | (3-(4-(3′-(2-hydroxyphenyl)- | | 14.10 (s, 1H), 9.00 (d, J = 2.5 Hz, |
| | | 5′,7′-dihydrospiro[azetidine- | | 1H), 8.46-8.35 (m, 2H), 8.11 (s, |
| | | 3,6′-pyrrolo[2,3- | | 1H), 7.91-7.82 (m, 1H), 7.55- |
| | | c]pyridazine]-1- | | 7.32 (m, 4H), 7.32-7.21 (m, 1H), |
| | | carbonyl)piperidin-1- | | 6.99-6.80 (m, 2H), 6.16 (s, 1H), |
| | | yl)isoxazol-5-yl)-3- | | 5.12 (d, J = 3.7 Hz, 1H), 4.98-4.87 |
| | | methylbutanoyl)-N-((S)-1-(4- | | (m, 1H), 4.47-4.23 (m, 4H), 4.10- |
| | | (4-methylthiazol-5- | | 3.99 (s, 2H), 3.78-3.52 (m, 6H), |
| | | yl)phenyl)ethyl)pyrrolidine-2- | | 3.51-3.39 (m, 1H), 2.97-2.69 (m, |
| | | carboxamide | | 2H), 2.47 (d, J = 1.3 Hz, 3H), 2.32- |
| | | | | 2.12 (m, 2H), 2.10-1.93 (m, 1H), |
| | | | | 1.89-1.52 (s, 5H), 1.42 (dd, J = |
| | | | | 22.2, 7.0 Hz, 3H), 0.96 (d, J = 6.4 |
| | | | | Hz, 3H), 0.82 (dd, J = 11.6, 6.6 Hz, |
| | | | | 3H). |
| 98 | B | (2S,4R)-4-hydroxy-1-((R)-2- | 818.40 | 1H NMR (400 MHz, Methanol-d4) δ |
| | | (3-(4-(3′-(2-hydroxyphenyl)- | | 8.88 (s, 1H), 7.97 (s, 1H), 7.80- |
| | | 5′,7′-dihydrospiro[azetidine- | | 7.73 (m, 1H), 7.48-7.35 (m, 4H), |
| | | 3,6′-pyrrolo[2,3-c]pyridazin]- | | 7.25 (t, J = 8.3 Hz, 1H), 6.96-6.88 |
| | | 1-yl)piperidin-1-yl)isoxazol-5- | | (m, 2H), 6.11 (s, 1H), 5.05-4.99 |
| | | yl)-3-methylbutanoyl)-N-((S)- | | (m, 1H), 4.55-4.39 (m, 2H), 3.84 |
| | | 1-(4-(4-methylthiazol-5- | | (dd, J = 10.8, 4.1 Hz, 1H), 3.70- |
| | | yl)phenyl)ethyl)pyrrolidine-2- | | 3.67 (m, 2H), 3.65-3.55 (m, 6H), |
| | | carboxamide | | 3.35 (d, J = 4.6 Hz, 3H), 2.90 (t, J = |
| | | | | 12.0 Hz, 2H), 2.48 (s, 2H), 2.23- |
| | | | | 2.13 (m, 1H), 2.01-1.87 (m, 1H), |
| | | | | 1.83 (d, J = 12.6 Hz, 2H), 1.59- |
| | | | | 1.52 (m, 3H), 1.39-1.35 (m, 3H), |
| | | | | 1.05 (d, J = 6.6 Hz, 3H), 0.90 (dd, |
| | | | | J = 12.1, 6.6 Hz, 4H). |
| 186 | B | (2S,4R)-4-hydroxy-1-((R)-2- | 779.25 | 1H NMR (300 MHz, DMSO-d6) δ |
| | | (3-(2-(3′-(2-hydroxyphenyl)- | | 14.19 (s, 1H), 8.99 (s, 1H), 8.42 (d, |
| | | 5′,7′-dihydrospiro[azetidine- | | J = 7.7 Hz, 1H), 8.20 (s, 1H), 8.05 |
| | | 3,6′-pyrrolo[2,3-c]pyridazin]- | | (s, 1H), 7.85 (d, J = 7.8 Hz, 1H), |
| | | 1-yl)ethoxy)isoxazol-5-yl)-3- | | 7.51-7.41 (m, 2H), 7.37 (d, J = 8.1 |
| | | methylbutanoyl)-N-((S)-1-(4- | | Hz, 2H), 7.31-7.19 (m, 1H), 6.89 |
| | | (4-methylthiazol-5- | | (dd, J = 8.3, 7.0 Hz, 2H), 6.10 (s, |
| | | yl)phenyl)ethyl)pyrrolidine-2- | | 1H), 5.11 (d, J = 3.6 Hz, 1H), 5.05- |
| | | carboxamide | | 4.86 (m, 1H), 4.37 (t, J = 7.9 Hz, |
| | | | | 1H), 4.29 (s, 1H), 4.15 (t, J = 5.2 |
| | | | | Hz, 2H), 3.69 (dd, J = 16.8, 10.3 |
| | | | | Hz, 2H), 3.49-3.47 (m, 5H), 3.24 |
| | | | | (d, J = 7.3 Hz, 2H), 2.79 (t, J = 5.3 |
| | | | | Hz, 2H), 2.46 (s, 3H), 2.28-2.16 |
| | | | | (m, 1H), 2.04 (t, J = 10.3 Hz, 1H), |
| | | | | 1.86-1.71 (m, 1H), 1.42 (d, J = |
| | | | | 24.0, 7.0 Hz, 3H), 0.97 (d, J = 6.6 |
| | | | | Hz, 3H), 0.88-0.77 (m, 3H). |
| 250 | A | (2S,4R)-4-hydroxy-1-((R)-2- | 862.20 | 1H NMR (400 MHz, DMSO-d6) δ |
| | | (3-(2-(2-(3-(3-(2- | | 13.95 (s, 1H), 12.50 (s, 1H), 8.98 (s, |
| | | hydroxyphenyl)-7H- | | 1H), 8.93-8.79 (m, 1H), 8.65 (s, |
| | | pyrrolo[2,3-c]pyridazin-5- | | 1H), 8.41 (d, J = 7.6 Hz, 1H), 8.19 |
| | | yl)azetidin-1-yl)-2- | | (s, 1H), 8.09 (dd, J = 8.4, 1.6 Hz, |
| | | oxoacetamido)ethoxy)isoxazol- | | 1H), 7.52-7.43 (m, 2H), 7.42- |
| | | 5-yl)-3-methylbutanoyl)-N- | | 7.33 (m, 2H), 7.32-7.26 (m, 1H), |
| | | ((S)-1-(4-(4-methylthiazol-5- | | 7.01-6.93 (m, 2H), 6.09 (s, 1H), |
| | | yl)phenyl)ethyl)pyrrolidine-2- | | 5.10 (d, J = 3.7 Hz, 1H), 5.01 (t, J = |
| | | carboxamide | | 9.5 Hz, 1H), 4.91 (t, J = 7.2 Hz, 1H), |
| | | | | 4.70-4.58 (m, 1H), 4.51 (t, J = 9.3 |
| | | | | Hz, 1H), 4.37 (t, J = 7.9 Hz, 1H), |
| | | | | 4.28-4.20 (m, 4H), 4.19-4.13 (m, |
| | | | | 1H), 3.74-3.67 (m, 2H),3.51- |
| | | | | 3.48 (m, 2H), 3.47-3.40 (m, |
| | | | | 1H),2.45 (s, 3H), 2.23 (s, 1H), 2.10- |
| | | | | 1.98 (m, 1H), 1.83-1.72 (m, 1H), |
| | | | | 1.37 (d, J = 7.0 Hz, 3H), 0.95 (dd, |
| | | | | J = 6.6, 2.4 Hz, 3H), 0.81 (dd, J = |
| | | | | 14.6, 6.7 Hz, 3H). |
| 266 | B | (2S,4R)-1-((R)-2-(4-chloro-3- | 825.1 | 1H NMR (400 MHz, DMSO-d6) δ |
| | | (2-(3-(3-(2-hydroxyphenyl)- | | 14.15 (s, 1H), 12.60 (s, 1H), 8.99 (s, |
| | | 7H-pyrrolo[2,3-c]pyridazin-6- | | 1H), 8.56 (s, 1H), 8.42 (d, J = 7.7 |
| | | yl)azetidin-1- | | Hz, 1H), 8.03 (d, J = 8.1 Hz, 1H), |
| | | yl)ethoxy)isoxazol-5-yl)-3- | | 7.44 (d, J = 8.0 Hz, 2H), 7.39-7.29 |
| | | methylbutanoyl)-4-hydroxy- | | (m, 2H), 7.33-7.25 (m, 1H), 6.96 |
| | | N-((S)-1-(4-(4-methylthiazol- | | (t, J = 8.4 Hz, 2H), 6.56 (s, 1H), |
| | | 5-yl)phenyl)ethyl)pyrrolidine- | | 5.14 (d, J = 3.9 Hz, 1H), 4.90 (q, J = |
| | | 2-carboxamide | | 7.5 Hz, 1H), 4.36 (t, J = 7.8 Hz, 1H), |
| | | | | 4.31-4.24 (m, 3H), 3.91 (q, J = 7.4 |
| | | | | Hz, 1H), 3.80-3.70 (m, 4H), 3.43 |
| | | | | (t, J = 6.8 Hz, 2H), 3.38 (s, 1H), |
| | | | | 2.91 (t, J = 5.2 Hz, 2H), 2.46 (s, |
| | | | | 3H), 2.43 (s, 1H), 2.02 (t, J = 9.7 |
| | | | | Hz, 1H), 1.79 (ddd, J = 12.7, 7.6, |
| | | | | 4.7 Hz, 1H), 1.38 (d, J = 7.1 Hz, |
| | | | | 3H), 0.99 (d, J = 6.5 Hz, 3H), 0.78 |
| | | | | (d, J = 6.7 Hz, 3H). |
| 267 | A | (2S,4R)-4-hydroxy-1-((R)-2- | −885.35 | 1H NMR (300 MHz, DMSO-d6) δ |
| | | (3-(4-(2-(3-(3-(2- | | 13.10 (br s, 1H), 9.00 (s, 1H), 8.69 |
| | | hydroxyphenyl)-7H- | | (s, 1H), 8.47 (s, 1H), 8.40 (d, J = |
| | | pyrrolo[2,3-c]pyridazin-5- | | 7.7 Hz, 1H), 7.89 (s, 1H), 7.51- |
| | | yl)azetidin-1-yl)-2- | | 7.33 (m, 5H), 7.11-6.97 (m, 2H), |
| | | oxoacetyl)piperazin-1- | | 6.19 (d, J = 1.7 Hz, 1H), 4.92 (t, J = |
| | | yl)isoxazol-5-yl)-3- | | 7.2 Hz, 1H), 4.76-4.62 (m, 1H), |
| | | methylbutanoyl)-N-((S)-1-(4- | | 4.60-4.48 (m, 1H), 4.43-4.18 (m, |
| | | (4-methylthiazol-5- | | 5H), 3.77-3.67 (m, 1H), 3.66- |
| | | yl)phenyl)ethyl)pyrrolidine-2- | | 3.54 (m, 6H), 3.25 (s, 4H), 2.46 (s, |
| | | carboxamide | | 3H), 2.28-2.12 (m, 1H), 2.11- |
| | | | | 1.94 (m, 1H), 1.88-1.73 (m, 1H), |
| | | | | 1.51-1.34 (m, 3H), 1.03-0.90 (m, |
| | | | | 3H), 0.88-0.74 (m, 3H). |
| 269 | A | (2S,4R)-4-hydroxy-1-((R)-2- | 873.5 | 1H NMR (300 MHz, DMSO-d6) δ |
| | | (3-(4-(2-(3-(3-(2- | | 14.16 (br s, 1H), 12.59 (br s, 1H), |
| | | hydroxyphenyl)-7H- | | 8.99 (s, 1H), 8.57 (s, 1H), 8.41 (d, |
| | | pyrrolo[2,3-c]pyridazin-6- | | J = 7.7 Hz, 1H), 8.08-8.00 (m, 1H), |
| | | yl)azetidin-1- | | 7.51-7.41 (m, 2H), 7.41-7.34 (m, |
| | | yl)acetyl)piperazin-1- | | 2H), 7.33-7.23 (m, 1H), 7.01- |
| | | yl)isoxazol-5-yl)-3- | | 6.89 (m, 2H), 6.58 (s, 1H), 6.21 (s, |
| | | methylbutanoyl)-N-((S)-1-(4- | | 1H), 5.12 (s, 1H), 4.92 (t, J = 7.2 |
| | | (4-methylthiazol-5- | | Hz, 1H), 4.37 (t, J = 7.8 Hz, 1H), |
| | | yl)phenyl)ethyl)pyrrolidine-2- | | 4.29 (s, 1H), 3.99-3.86 (m, 1H), |
| | | carboxamide | | 3.83-3.67 (m, 3H), 3.66-3.50 (m, |
| | | | | 6H), 3.50-3.39 (m, 3H), 3.28- |
| | | | | 3.11 (m, 5H), 2.46 (s, 3H), 2.26- |
| | | | | 2.14 (m, 1H), 2.10-1.97 (m, 1H), |
| | | | | 1.87-1.72 (m, 1H), 1.51-1.33 (m, |
| | | | | 3H), 1.05-0.91 (m, 3H), 0.89- |
| | | | | 0.75 (m, 3H). |
| 239 | A | N-(2-((5-((R)-1-((2S,4R)-4- | 779.5 | 1H NMR (300 MHz, DMSO-d6) δ |
| | | hydroxy-2-(((S)-1-(4-(4- | | 13.34 (s, 1H), 9.28 (t, J = 5.5 Hz, |
| | | methylthiazol-5- | | 1H), 8.99 (s, 1H), 8.85 (s, 1H), 8.41 |
| | | yl)phenyl)ethyl)carbamoyl)pyrrolidin- | | (d, J = 7.6 Hz, 1H), 7.98 (d, J = 7.7 |
| | | 1-yl)-3-methyl-1- | | Hz, 1H), 7.47-7.41 (m, 2H), 7.39- |
| | | oxobutan-2-yl)isoxazol-3- | | 7.32 (m, 4H), 7.05-6.97 (m, 2H), |
| | | yl)oxy)ethyl)-3-(2- | | 6.05 (d, J = 47.8 Hz, 1H), 4.89 (q, |
| | | hydroxyphenyl)-7H- | | J = 7.0 Hz, 1H), 4.46-4.31 (m, 3H), |
| | | pyrrolo[2,3-c]pyridazine-6- | | 4.28 (s, 1H), 3.84-3.55 (m, 7H), |
| | | carboxamide | | 2.45 (s, 3H), 2.25-2.20 (m, 1H), |
| | | | | 2.11-1.97 (m, 1H), 1.84-1.73 (m, |
| | | | | 1H), 1.41 (d, J = 7.0 Hz, 3H), 0.96 |
| | | | | (d, J = 6.7 Hz, 3H), 0.80 (d, J = 6.6 |
| | | | | Hz, 3H). |
| 240 | A | (2S,4R)-4-hydroxy-1-((R)-2- | 862.5 | 1H NMR (400 MHz, Methanol-d4) δ |
| | | (3-(2-(2-(3-(3-(2- | | 8.89-8.81 (m, 1H), 8.47 (s, 1H), |
| | | hydroxyphenyl)-7H- | | 7.97-7.89 (m, 1H), 7.47-7.34 (m, |
| | | pyrrolo[2,3-c]pyridazin-6- | | 4H), 7.32-7.24 (m, 1H), 7.01- |
| | | yl)azetidin-1-yl)-2- | | 6.92 (m, 2H), 6.67 (s, 1H), 6.01 (s, |
| | | oxoacetamido)ethoxy)isoxazol- | | 1H), 5.14-5.05 (m, 1H), 5.05- |
| | | 5-yl)-3-methylbutanoyl)-N- | | 4.99 (m, 1H), 4.83-4.80 (m, 1H), |
| | | ((S)-1-(4-(4-methylthiazol-5- | | 4.63-4.47 (m, 2H), 4.46-4.22 (m, |
| | | yl)phenyl)ethyl)pyrrolidine-2- | | 5H), 3.87-3.78 (m, 1H), 3.74- |
| | | carboxamide | | 3.57 (m, 4H), 2.49-2.30 (m, 4H), |
| | | | | 2.23-2.13 (m, 1H), 2.01-1.89 (m, |
| | | | | 1H), 1.60-1.47 (m, 3H), 1.29 (s, |
| | | | | 1H), 1.07-1.01 (m, 3H), 0.94- |
| | | | | 0.85 (m, 3H). |
| 249 | A | (2S,4R)-4-hydroxy-1-((R)-2- | 848.50 | 1H NMR (300 MHz, DMSO-d6) δ |
| | | (3-(2-(2-(3-(3-(2- | | 14.17 (s, 1H), 12.57 (s, 1H), 8.99 (s, |
| | | hydroxyphenyl)-7H- | | 1H), 8.57 (d, J = 2.9 Hz, 1H), 8.41 |
| | | pyrrolo[2,3-c]pyridazin-6- | | (d, J = 7.6 Hz, 1H), 8.04 (d, J = 8.9 |
| | | yl)azetidin-1- | | Hz, 2H), 7.44 (d, J = 8.3 Hz, 2H), |
| | | yl)acetamido)ethoxy)isoxazol- | | 7.36 (d, J = 8.2 Hz, 2H), 7.33-7.26 |
| | | 5-yl)-3-methylbutanoyl)-N- | | (m, 1H), 6.97 (d, J = 7.7 Hz, 2H), |
| | | ((S)-1-(4-(4-methylthiazol-5- | | 6.58 (d, J = 2.8 Hz, 1H), 6.10 (s, |
| | | yl)phenyl)ethyl)pyrrolidine-2- | | 1H), 5.12 (s, 1H), 4.96-4.85 (m, |
| | | carboxamide | | 1H), 4.37 (t, J = 7.9 Hz, 1H), 4.28 |
| | | | | (s, 1H), 4.21 (t, J = 5.5 Hz, 2H), |
| | | | | 3.91 (q, J = 7.2 Hz, 1H), 3.76 (t, J = |
| | | | | 7.4 Hz, 2H), 3.65 (d, J = 9.9 Hz, |
| | | | | 1H), 3.52-3.41 (m, 6H), 3.18 (s, |
| | | | | 2H), 2.46 (s, 3H), 2.27-2.12 (m, |
| | | | | 1H), 2.01 (d, J = 9.2 Hz, 1H), 1.79 |
| | | | | (d, J = 6.4 Hz, 1H), 1.41 (d, J = |
| | | | | 21.5, 7.0 Hz, 3H), 0.95 (d, J = 6.4 |
| | | | | Hz, 3H), 0.77 (d, J = 6.7 Hz, 3H). |
| 229 | B | (2S,4R)-4-hydroxy-1-((R)-2- | 819.2 | 1H NMR (400 MHz, Methanol-d4) δ |
| | | (3-(3-(3-(3-(2- | | 8.86 (s, 1H), 8.45 (s, 1H), 7.96 (d, |
| | | hydroxyphenyl)-5-methyl- | | J = 7.9 Hz, 1H), 7.46-7.35 (m, 3H), |
| | | 7H-pyrrolo[2,3-c]pyridazin-6- | | 7.35-7.23 (m, 2H), 7.03-6.94 (m, |
| | | yl)azetidin-1- | | 2H), 5.91 (s, 1H), 5.05-4.99 (m, |
| | | yl)propoxy)isoxazol-5-yl)-3- | | 1H), 4.57-4.34 (m, 4H), 4.28- |
| | | methylbutanoyl)-N-((S)-1-(4- | | 4.15 (m, 2H), 4.11-4.00 (m, 2H), |
| | | (4-methylthiazol-5- | | 3.83-3.64 (m, 3H), 3.42 (d, J = 9.9 |
| | | yl)phenyl)ethyl)pyrrolidine-2- | | Hz, 1H), 3.25-3.16 (m, 2H), 2.46 |
| | | carboxamide | | (s, 3H), 2.41-2.37 (m, 1H), 2.33 |
| | | | | (d, J = 15.6 Hz, 3H), 2.23 (d, J = 5.9 |
| | | | | Hz, 1H), 2.08-1.91 (m, 3H), 1.50 |
| | | | | (d, 3H), 1.08 (d, J = 6.6 Hz, 3H), |
| | | | | 0.99 (d, J = 6.1 Hz, 3H). |
| 298 | A | N1-((S)-1-((2S,4R)-4- | 805.40 | 1H NMR (300 MHz, DMSO-d6) δ |
| | | hydroxy-2-((4-(4- | | 14.15 (s, 1H), 12.47-11.88 (m, |
| | | methylthiazol-5- | | 1H), 8.98 (s, 1H), 8.67-8.49 (m, |
| | | yl)benzyl)carbamoyl)pyrrolidin- | | 3H), 8.09-7.98 (m, 1H), 7.91 (d, |
| | | 1-yl)-3,3-dimethyl-1- | | J = 9.3 Hz, 1H), 7.50-7.35 (m, 4H), |
| | | oxobutan-2-yl)-N4-(3-(3-(2- | | 7.54-7.29 (m, 1H), 7.01-6.91 (m, |
| | | hydroxyphenyl)-7H- | | 2H), 6.41 (s, 1H), 5.14 (d, J = 3.4 |
| | | pyrrolo[2,3-c]pyridazin-6- | | Hz, 1H), 4.54 (d, J = 9.3 Hz, 1H), |
| | | yl)bicyclo[1.1.1]pentan-1- | | 4.51-4.40 (m, 2H), 4.36 (s, 1H), |
| | | yl)succinamide | | 4.34-4.15 (m, 1H), 3.75-3.57 (m, |
| | | | | 2H), 2.47-2.35 (m, 10H), 2.33- |
| | | | | 2.24 (s, 3H), 2.11-2.01 (m, 1H), |
| | | | | 1.99-1.82 (m, 1H), 0.95 (s, 9H). |
| 306 | A | (2S,4R)-4-hydroxy-1-((S)-2- | 751.20 | 1H), 8.64 (t, J = 6.0 Hz, 1H), 8.55 |
| | | (2-(3-(3-(2-hydroxyphenyl)- | | (s, 1H), 8.02 (d, J = 8.0 Hz, 1H), |
| | | 7H-pyrrolo[2,3-c]pyridazin-6- | | 7.95 (d, J = 9.7 Hz, 1H), 7.41 (s, |
| | | yl)azetidin-1-yl)-2- | | 4H), 7.27 (t, J = 7.6 Hz, 1H), 6.98- |
| | | oxoacetamido)-3,3- | | 6.90 (m, 2H), 6.64 (d, J = 6.1 Hz, |
| | | dimethylbutanoyl)-N-(4-(4- | | 1H), 5.18 (d, J = 3.4 Hz, 1H), 4.94 |
| | | methylthiazol-5- | | (t, J = 9.3 Hz, 1H), 4.70 (dt, J = |
| | | yl)benzyl)pyrrolidine-2- | | 12.4, 6.3 Hz, 1H), 4.46 (ddd, J = |
| | | carboxamide | | 13.6, 9.4, 4.5 Hz, 3H), 4.40-4.31 |
| | | | | (m, 2H), 4.31-4.18 (m, 3H), 3.69- |
| | | | | 3.56 (m, 2H), 2.44 (s, 3H), 2.06 (d, |
| | | | | J = 9.0 Hz, 1H), 1.90 (s, 1H), 0.95 |
| | | | | (d, J = 9.4 Hz, 9H). |
| 236 | A | (2S,4R)-4-hydroxy-1-((R)-2- | 858.50 | 1H NMR (400 MHz, DMSO-d6) δ |
| | | (3-(4-(3-(3-(2- | | 13.92-13.86 (m, 1H), 12.65- |
| | | hydroxyphenyl)-7H- | | 12.36 (m, 1H), 8.98 (d, J = 10.6 Hz, |
| | | pyrrolo[2,3-c]pyridazin-5- | | 1H), 8.60 (s, 1H), 8.24 (d, J = 7.8 |
| | | yl)azetidine-1- | | Hz, 1H), 8.14 (s, 1H), 8.05 (d, J = |
| | | carbonyl)piperidin-1- | | 7.8 Hz, 1H), 7.51-7.26 (m, 5H), |
| | | yl)isoxazol-5-yl)-3- | | 7.01-6.93 (m, 2H), 6.18-6.14 (m, |
| | | methylbutanoyl)-N-((S)-1-(4- | | 1H), 5.13-4.82 (m, 2H), 4.75- |
| | | (4-methylthiazol-5- | | 4.65 (m, 1H), 4.46-4.39 (m, 1H), |
| | | yl)phenyl)ethyl)pyrrolidine-2- | | 4.39-4.30 (m, 2H), 4.30-4.22 (m, |
| | | carboxamide | | 1H), 4.20-4.10 (m, 1H), 4.08- |
| | | | | 4.00 (m, 1H), 3.72-3.57 (m, 3H), |
| | | | | 3.56-3.39 (m, 3H), 2.90-2.70 (m, |
| | | | | 2H), 2.44 (s, 3H), 2.30-1.74 (m, |
| | | | | 3H), 1.74-1.64 (m, 2H), 1.64- |
| | | | | 1.51 (m, 2H), 1.35 (d, J = 7.0 Hz, |
| | | | | 3H), 0.96 (d, J = 6.6 Hz, 3H), 0.82 |
| | | | | (d, J = 6.6 Hz, 3H). |
| 252 | A | (2S,4R)-4-hydroxy-1-((S)-2- | 804.60 | 1H NMR (300 MHz, DMSO-d6) δ |
| | | (3-(4-(3-(2-hydroxyphenyl)- | | 13.46 (s, 1H), 9.05-8.93 (m, 1H), |
| | | 7H-pyrrolo[2,3-c]pyridazine- | | 8.77 (s, 1H), 8.29 (d, J = 7.8 Hz, |
| | | 6-carbonyl)piperazin-1- | | 1H), 8.00-7.84 (m, 1H), 7.61- |
| | | yl)isoxazol-5-yl)-3- | | 7.21 (m, 6H), 7.13-6.90 (m, 3H), |
| | | methylbutanoyl)-N-((S)-1-(4- | | 6.31-6.20 (m, 1H), 5.09-4.83 (m, |
| | | (4-methylthiazol-5- | | 1H), 4.61-4.38 (m, 1H), 4.28 (s, |
| | | yl)phenyl)ethyl)pyrrolidine-2- | | 2H), 3.99-3.60 (m, 6H), 3.37- |
| | | carboxamide | | 3.24 (m, 5H), 2.47 (s, 1H), 2.44 (s, |
| | | | | 3H), 2.11-1.99 (m, 1H), 1.87- |
| | | | | 1.73 (m, 1H), 1.42 (dd, J = 32.6, 7.0 |
| | | | | Hz, 3H), 0.97 (d, J = 6.6 Hz, 2H), |
| | | | | 0.89-0.74 (m, 4H). |
| 261 | B | (2S,4R)-4-hydroxy-1-((R)-2- | 805.40 | 1H NMR (400 MHz, DMSO-d6) δ |
| | | (3-(2-((R)-2-(3-(2- | | 14.21 (s, 1H), 12.52 (s, 1H), 8.99 (s, |
| | | hydroxyphenyl)-7H- | | 1H), 8.55 (s, 1H), 8.41 (d, J = 7.6 |
| | | pyrrolo[2,3-c]pyridazin-6- | | Hz, 1H), 8.05-7.96 (m, 1H), 7.44 |
| | | yl)pyrrolidin-1- | | (d, J = 8.2 Hz, 2H), 7.39-7.33 (m, |
| | | yl)ethoxy)isoxazol-5-yl)-3- | | 2H), 7.33-7.25 (m, 1H), 7.00- |
| | | methylbutanoyl)-N-((S)-1-(4- | | 6.88 (m, 2H), 6.55 (d, J = 9.4 Hz, |
| | | (4-methylthiazol-5- | | 1H), 6.04 (s, 1H), 5.08 (d, J = 3.5 |
| | | yl)phenyl)ethyl)pyrrolidine-2- | | Hz, 1H), 4.92 (q, J = 7.1 Hz, 1H), |
| | | carboxamide | | 4.35 (t, J = 7.9 Hz, 1H), 4.28-4.19 |
| | | | | (m, 3H), 3.88-3.81 (m, 1H), 3.71- |
| | | | | 3.62 (m, 1H), 3.55 (d, J = 23.5, 11.4 |
| | | | | Hz, 1H), 3.40 (d, J = 10.6 Hz, 1H), |
| | | | | 3.05-2.97 (m, 1H), 2.75-2.67 (m, |
| | | | | 1H), 2.56-2.54 (m, 1H), 2.45 (d, |
| | | | | J = 4.3 Hz, 4H), 2.30-2.25 (m, 1H), |
| | | | | 2.23-2.13 (m, 1H), 2.07-1.97 (m, |
| | | | | 1H), 1.94-1.83 (m, 3H), 1.82- |
| | | | | 1.71 (m, 1H), 1.39 (d, J = 10.4, 7.1 |
| | | | | Hz, 3H), 0.90 (d, J = 6.6, 4.0 Hz, |
| | | | | 3H), 0.68 (d, J = 6.6, 2.6 Hz, 3H). |
| 297 | B | (2S,4R)-4-hydroxy-1-((S)-2- | 779.60 | 1H NMR (300 MHz, DMSO-d6) δ |
| | | (5-(3-(3-(2-hydroxyphenyl)- | | 14.12 (s, 1H), 14.50 (s, 1H), 8.99 (s, |
| | | 7H-pyrrolo[2,3-c]pyridazin-6- | | 1H), 8.56 (d, J = 8.5 Hz, 2H), 8.08- |
| | | yl)azetidin-1- | | 7.99 (m, 1H), 7.88 (d, J = 9.3 Hz, |
| | | yl)pentanamido)-3,3- | | 1H), 7.47-7.35 (m, 4H), 7.30 (td, |
| | | dimethylbutanoyl)-N-(4-(4- | | J = 7.7, 7.1, 1.6 Hz, 1H), 6.96 (t, J = |
| | | methylthiazol-5- | | 8.3 Hz, 2H), 6.59 (s, 1H), 5.20 (s, |
| | | yl)benzyl)pyrrolidine-2- | | 1H), 4.56 (d, J = 9.3 Hz, 1H), 4.51- |
| | | carboxamide | | 4.33 (m, 3H), 4.22 (dd, J = 15.8, 5.4 |
| | | | | Hz, 1H), 3.98-3.63 (m, 5H), 2.45 |
| | | | | (s, 3H), 2.21-1.83 (m, 6H), 1.53 |
| | | | | (s, 2H), 1.33 (s, 2H), 1.24 (s, 1H), |
| | | | | 0.95 (s, 9H). |
| 205 | A | (2S,4R)-4-hydroxy-1-((R)-2- | 858.55 | 1H NMR (400 MHz, DMSO-d6) δ |
| | | (3-(4-(3-(2-hydroxyphenyl)- | | 14.32-14.15 (m, 1H), 12.46 (s, |
| | | 6,7,8,9-tetrahydro-5H- | | 1H), 9.01-8.85 (m, 1H), 8.73- |
| | | pyrido[3′,4′:4,5]pyrrolo[2,3- | | 8.56 (m, 1H), 8.23 (d, J = 7.8 Hz, |
| | | c]pyridazine-6- | | 1H), 8.07 (dd, J = 31.8, 7.9 Hz, 1H), |
| | | carbonyl)piperidin-1- | | 7.49-7.26 (m, 5H), 6.99-6.91 (m, |
| | | yl)isoxazol-5-yl)-3- | | 2H), 6.19-6.13 (m, 1H), 5.13- |
| | | methylbutanoyl)-N-((S)-1-(4- | | 4.83 (m, 3H), 4.74 (s, 1H), 4.55- |
| | | (4-methylthiazol-5- | | 4.37 (m, 1H), 4.27 (s, 1H), 3.94 (s, |
| | | yl)phenyl)ethyl)pyrrolidine-2- | | 2H), 3.73-3.60 (m, 3H), 3.56- |
| | | carboxamide | | 3.38 (m, 2H), 3.09-2.78 (m, 5H), |
| | | | | 2.49-2.41 (m, 3H), 2.33 (p, J = |
| | | | | 3.7, 1.8 Hz, 1H), 2.27-1.87 (m, |
| | | | | 1H), 1.75-1.59 (m, 5H), 1.49- |
| | | | | 1.32 (m, 3H), 0.96 (d, J = 6.3 Hz, |
| | | | | 2H), 0.87-0.69 (m, 4H). |
| 210 | B | (2S,4R)-4-hydroxy-1-((R)-2- | 817.55 | 1H NMR (300 MHz, DMSO-d6) δ |
| | | (3-(2-((1R,5S,6S)-6-(3-(2- | | 14.13 (s, 1H), 12.32 (s, 1H), 8.99 (s, |
| | | hydroxyphenyl)-7H- | | 1H), 8.72 (s, 1H), 8.42 (d, J = 7.7 |
| | | pyrrolo[2,3-c]pyridazin-5-yl)- | | Hz, 1H), 8.30-8.10 (m, 1H), 7.76 |
| | | 3-azabicyclo[3.1.0]hexan-3- | | (s, 1H), 7.47-7.41 (m, 2H), 7.37 |
| | | yl)ethoxy)isoxazol-5-yl)-3- | | (d, J = 8.2 Hz, 2H), 7.34-7.21 (m, |
| | | methylbutanoyl)-N-((S)-1-(4- | | 1H), 7.01-6.92 (m, 2H), 6.04 (d, J = |
| | | (4-methylthiazol-5- | | 51.9 Hz, 1H), 5.11 (d, J = 3.6 Hz, |
| | | yl)phenyl)ethyl)pyrrolidine-2- | | 1H), 5.03-4.81 (m, 1H), 4.39 (t, |
| | | carboxamide | | 1H), 4.33-4.20 (m, 3H), 3.75- |
| | | | | 3.59 (m, 2H), 3.46 (d, J = 10.3 Hz, |
| | | | | 1H), 3.31-3.23 (m,3H), 2.91- |
| | | | | 2.83 (m, 2H), 2.46 (s, 3H), 2.41- |
| | | | | 2.36 (m, 1H), 2.29-2.16 (m, 1H), |
| | | | | 2.13-1.95 (m, 1H), 1.91-1.83 (m, |
| | | | | 2H), 1.83-1.66 (m, 1H), 1.42 (d, |
| | | | | J = 24.6, 6.9 Hz, 3H), 1.24 (s, 1H), |
| | | | | 0.97 (d, J = 6.4, 3.7 Hz, 3H), 0.83 |
| | | | | (d, J = 10.5, 6.5 Hz, 3H). |
| 227 | B | (2S,4R)-4-hydroxy-1-((R)-2- | 805.35 | 1H NMR (300 MHz, DMSO-d6) δ |
| | | (3-(2-((S)-2-(3-(2- | | 13.90 (s, 1H), 12.42 (s, 1H), 8.98 (s, |
| | | hydroxyphenyl)-7H- | | 1H), 8.67 (s, 1H), 8.39 (d, J = 7.8 |
| | | pyrrolo[2,3-c]pyridazin-5- | | Hz, 1H), 8.02 (d, J = 7.7 Hz, 1H), |
| | | yl)pyrrolidin-1- | | 7.94 (s, 1H), 7.48-7.25 (m, 5H), |
| | | yl)ethoxy)isoxazol-5-yl)-3- | | 7.03-6.92 (m, 2H), 5.85 (s, 1H), |
| | | methylbutanoyl)-N-((S)-1-(4- | | 5.08 (d, J = 3.6 Hz, 1H), 4.91 (t, J = |
| | | (4-methylthiazol-5- | | 7.2 Hz, 1H), 4.34 (t, J = 7.9 Hz, 1H), |
| | | yl)phenyl)ethyl)pyrrolidine-2- | | 4.29-4.10 (m, 3H), 3.82-3.51 (m, |
| | | carboxamide | | 3H), 3.43-3.35 (m, 2H), 2.99-2.86 |
| | | | | (m, 1H), 2.55-2.53 (m, 1H), 2.46 |
| | | | | (s, 3H), 2.41-2.30 (m,1H), 2.23- |
| | | | | 2.10 (m, 2H), 2.04-1.68 (m, 5H), |
| | | | | 1.37 (d, J = 7.0 Hz, 3H), 0.92 (d, J = |
| | | | | 6.5 Hz, 3H), 0.75 (d, J = 6.5 Hz, |
| | | | | 3H). |
| 228 | B | (2S,4R)-4-hydroxy-1-((R)-2- | 805.35 | 1H NMR (300 MHz, DMSO-d6) δ |
| | | (3-(2-((R)-2-(3-(2- | | 13.90 (s, 1H), 12.42 (s, 1H), 8.98 (s, |
| | | hydroxyphenyl)-7H- | | 1H), 8.67 (s, 1H), 8.39 (d, J = 7.8 |
| | | pyrrolo[2,3-c]pyridazin-5- | | Hz, 1H), 8.02 (d, J = 7.7 Hz, 1H), |
| | | yl)pyrrolidin-1- | | 7.94 (s, 1H), 7.48-7.25 (m, 5H), |
| | | yl)ethoxy)isoxazol-5-yl)-3- | | 7.03-6.92 (m, 2H), 5.85 (s, 1H), |
| | | methylbutanoyl)-N-((S)-1-(4- | | 5.08 (d, J = 3.6 Hz, 1H), 4.91 (t, J = |
| | | (4-methylthiazol-5- | | 7.2 Hz, 1H), 4.34 (t, J = 7.9 Hz, 1H), |
| | | yl)phenyl)ethyl)pyrrolidine-2- | | 4.29-4.10 (m, 3H), 3.82-3.51 (m, |
| | | carboxamide | | 3H), 3.43-3.35 (m, 2H), 2.99-2.86 |
| | | | | (m, 1H), 2.55-2.53 (m, 1H), 2.46 |
| | | | | (s, 3H), 2.41-2.30 (m, 1H), 2.23- |
| | | | | 2.10 (m, 2H), 2.04-1.68 (m, 5H), |
| | | | | 1.37 (d, J = 7.0 Hz, 3H), 0.92 (d, J = |
| | | | | 6.5 Hz, 3H), 0.75 (d, J = 6.5 Hz, |
| | | | | 3H). |
| 191 | A | (S)-N-(2-(((S)-1-((2S,4R)-4- | 779.45 | 1H NMR (400 MHz, DMSO-d6) δ |
| | | hydroxy-2-((4-(4- | | 14.41 (s, 1H), 12.26 (s, 1H), 8.96 (s, |
| | | methylthiazol-5- | | 1H), 8.60 (t, J = 6.1 Hz, 1H), 8.51 |
| | | yl)benzyl)carbamoyl)pyrrolidin- | | (s, 1H), 8.28 (t, J = 5.9 Hz, 1H), |
| | | 1-yl)-3,3-dimethyl-1- | | 8.06 (dd, J = 8.0, 1.6 Hz, 1H), 7.88 |
| | | oxobutan-2-yl)amino)-2- | | (d, J = 9.3 Hz, 1H), 7.46-7.35 (m, |
| | | oxoethyl)-3-(2- | | 4H), 7.27 (td, J = 8.3, 7.7, 1.6 Hz, |
| | | hydroxyphenyl)-6,7,8,9- | | 1H), 6.98-6.90 (m, 2H), 5.17 (d, |
| | | tetrahydro-5H- | | J = 3.5 Hz, 1H), 4.56 (d, J = 9.4 Hz, |
| | | pyridazino[3,4-b]indole-6- | | 1H), 4.49-4.40 (m, 2H), 4.39- |
| | | carboxamide | | 4.34 (m, 1H), 4.22 (dd, J = 15.8, 5.4 |
| | | | | Hz, 1H), 3.84 (t, 2H), 3.70 (dd, J = |
| | | | | 10.6, 4.0 Hz, 1H), 3.63 (d, J = 10.6 |
| | | | | Hz, 1H), 3.01-2.86 (m, 3H), 2.81 |
| | | | | (d, J = 10.4 Hz, 1H), 2.78-2.71 (m, |
| | | | | 1H), 2.43 (s, 3H), 2.19-2.11 (m, |
| | | | | 1H), 2.10-2.02 (m, 1H), 1.99- |
| | | | | 1.87 (m, 2H), 0.95 (s, 9H). |
| 194 | A | (2S,4R)-4-hydroxy-1-((R)-2- | 805.45 | 1H NMR (400 MHz, DMSO-d6) δ |
| | | (3-(2-(3-(2-hydroxyphenyl)- | | 14.30-14.25 (m, 1H), 12.23 (s, |
| | | 5,7,8,9-tetrahydro-6H- | | 1H), 9.00-8.95 (m, 1H), 8.72 (s, |
| | | pyrido[3′,4′:4,5]pyrrolo[2,3- | | 1H), 8.41 (d, J = 7.7 Hz, 1H), 8.07 |
| | | c]pyridazin-6-yl)-2- | | (dd, J = 19.0, 8.0 Hz, 1H), 7.47- |
| | | oxoethoxy)isoxazol-5-yl)-3- | | 7.40 (m, 2H), 7.40-7.33 (m, 2H), |
| | | methylbutanoyl)-N-((S)-1-(4- | | 7.32-7.27 (m, 1H), 7.00-6.91 (m, |
| | | (4-methylthiazol-5- | | 2H), 6.21-6.16 (m, 1H), 5.13 (d, |
| | | yl)phenyl)ethyl)pyrrolidine-2- | | J = 18.5 Hz, 3H), 4.90 (q, J = 7.4 Hz, |
| | | carboxamide | | 1H), 4.79-4.73 (m, 2H), 4.38 (t, |
| | | | | J = 7.8 Hz, 1H), 4.28 (s, 1H), 3.96- |
| | | | | 3.78 (m, 2H), 3.75-3.63 (m, 2H), |
| | | | | 3.49-3.39 (m, 1H), 3.10-2.92 (m, |
| | | | | 2H), 2.48-2.42 (m, 3H), 2.31- |
| | | | | 2.14 (m, 1H), 2.05-2.00 (m, 1H), |
| | | | | 1.84-1.73 (m, 1H), 1.45-1.34 (m, |
| | | | | 3H), 0.99-0.93 (m, 3H), 0.85- |
| | | | | 0.76 (m, 3H). |
| 197 | A | (R)-N-(2-(((S)-1-((2S,4R)-4- | 779.40 | 1H NMR (400 MHz, DMSO-d6) δ |
| | | hydroxy-2-((4-(4- | | 14.40 (s, 1H), 12.25 (s, 1H), 8.96 (s, |
| | | methylthiazol-5- | | 1H), 8.59 (t, J = 6.0 Hz, 1H), 8.51 |
| | | yl)benzyl)carbamoyl)pyrrolidin- | | (s, 1H), 8.28 (t, J = 5.8 Hz, 1H), |
| | | 1-yl)-3,3-dimethyl-1- | | 8.07 (dd, J = 8.0, 1.6 Hz, 1H), 7.83 |
| | | oxobutan-2-yl)amino)-2- | | (d, J = 9.3 Hz, 1H), 7.48-7.34 (m, |
| | | oxoethyl)-3-(2- | | 4H), 7.28 (ddd, J = 8.6, 7.1, 1.6 Hz, |
| | | hydroxyphenyl)-6,7,8,9- | | 1H), 6.99-6.90 (m, 2H), 5.16 (d, |
| | | tetrahydro-5H- | | J = 3.4 Hz, 1H), 4.56 (d, J = 9.4 Hz, |
| | | pyridazino[3,4-b]indole-6- | | 1H), 4.49-4.39 (m, 2H), 4.39- |
| | | carboxamide | | 4.35 (m, 1H), 4.23 (dd, J = 15.9, 5.5 |
| | | | | Hz, 1H), 3.92-3.76 (m, 2H), 3.72- |
| | | | | 3.59 (m, 2H), 3.00-2.87 (m, 3H), |
| | | | | 2.83-2.71 (m, 2H), 2.43 (s, 3H), |
| | | | | 2.20-2.13 (m, 1H), 2.09-2.01 (m, |
| | | | | 1H), 1.98-1.87 (m, 2H), 0.95 (s, |
| | | | | 9H). |
| 201 | A | (2S,4R)-4-hydroxy-1-((S)-2- | 803.45 | 1H NMR (400 MHz, DMSO-d6) δ |
| | | (3-(1-(3-(2-hydroxyphenyl)- | | 13.61 (s, 1H), 13.02 (s, 1H), 9.01- |
| | | 7H-pyrrolo[2,3-c]pyridazine- | | 8.89 (m, 1H), 8.68 (s, 1H), 8.27 (d, |
| | | 6-carbonyl)piperidin-4- | | J = 7.8 Hz, 1H), 8.05-7.98 (m, |
| | | yl)isoxazol-5-yl)-3- | | 1H), 7.51-7.41 (m, 1H), 7.41- |
| | | methylbutanoyl)-N-((S)-1-(4- | | 7.26 (m, 4H), 7.02-6.92 (m, 2H), |
| | | (4-methylthiazol-5- | | 6.89-6.83 (m, 1H), 6.43-6.36 (m, |
| | | yl)phenyl)ethyl)pyrrolidine-2- | | 1H), 5.13-4.79 (m, 2H), 4.61- |
| | | carboxamide | | 4.35 (m, 2H), 4.27 (s, 1H), 4.03- |
| | | | | 3.98 (m, 1H), 3.87 (d, J = 8.7 Hz, |
| | | | | 1H), 3.61-3.54 (m, 1H), 3.54- |
| | | | | 3.38 (m, 2H), 3.17-2.97 (m, 2H), |
| | | | | 2.49-2.41 (m, 3H), 2.32-2.22 (m, |
| | | | | 1H), 2.10-1.86 (m, 3H), 1.84- |
| | | | | 1.74 (m, 1H), 1.68 (s, 2H), 1.41 (dd, |
| | | | | J = 45.5, 7.0 Hz, 3H), 0.98 (d, J = |
| | | | | 6.6 Hz, 3H), 0.87-0.71 (m, 3H). |
| 178 | A | (2S,4R)-4-hydroxy-1-((R)-2- | 775.25 | 1H NMR (300 MHz, DMSO-d6) δ |
| | | (3-(1-(3-(2-hydroxyphenyl)- | | 13.43 (s, 1H), 8.99 (s, 1H), 8.71 (s, |
| | | 7H-pyrrolo[2,3-c]pyridazine- | | 1H), 8.38 (m, 1H), 8.03 (d, J = 8.0 |
| | | 6-carbonyl)azetidin-3- | | Hz, 1H), 7.60-7.16 (m, 5H), 7.12- |
| | | yl)isoxazol-5-yl)-3- | | 6.85 (m, 3H), 6.59 (s, 1H), 5.25- |
| | | methylbutanoyl)-N-((S)-1-(4- | | 4.79 (m, 3H), 4.76-4.49 (m, 2H), |
| | | (4-methylthiazol-5- | | 4.45-4.01 (m, 4H), 3.93-3.54 (m, |
| | | yl)phenyl)ethyl)pyrrolidine-2- | | 2H), 3.49-3.40 (m, 1H), 2.46-2.40 |
| | | carboxamide | | (m, 4H), 2.10-1.95 (m, 1H), 1.88- |
| | | | | 1.69 (m, 1H), 1.38 (d, J = 7.0 Hz, |
| | | | | 3H), 1.00 (d, J = 6.4 Hz, 3H), 0.82 |
| | | | | (d, J = 7.0 Hz, 3H). |
| 180 | A | N1-((S)-1-((2S,4R)-4- | 807.00 | 1H NMR (300 MHz, Methanol-d4) δ |
| | | hydroxy-2-((4-(4- | | 8.86 (s, 1H), 8.35 (s, 1H), 8.01- |
| | | methylthiazol-5- | | 7.95 (m, 1H), 7.47-7.37 (m, 4H), |
| | | yl)benzyl)carbamoyl)pyrrolidin- | | 7.31-7.26 (m, 1H), 7.00-6.94 (m, |
| | | 1-yl)-3,3-dimethyl-1- | | 2H), 4.64-4.50 (m, 4H), 4.37- |
| | | oxobutan-2-yl)-N5-((R)-3-(2- | | 4.25 (m, 2H), 3.96-3.66 (m, 2H), |
| | | hydroxyphenyl)-6,7,8,9- | | 3.16 (dd, J = 15.4, 5.3 Hz, 1H), 3.01 |
| | | tetrahydro-5H- | | (t, J = 6.0 Hz, 2H), 2.69 (dd, J = |
| | | pyridazino[3,4-b]indol-6- | | 15.5, 8.2 Hz, 1H), 2.46 (s, 3H), 2.38- |
| | | yl)glutaramide | | 2.19 (m, 6H), 2.14-1.92 (m, 4H), |
| | | | | 1.06 (d, J = 7.9 Hz, 9H). |
| 189 | A | (S)-N-(6-(((S)-1-((2S,4R)-4- | 835.60 | 1H NMR (300 MHz, Methanol-d4) δ |
| | | hydroxy-2-((4-(4- | | 8.86 (s, 1H), 8.30 (s, 1H), 7.96- |
| | | methylthiazol-5- | | 7.93 (m, 1H), 7.47-7.38 (m, 4H), |
| | | yl)benzyl)carbamoyl)pyrrolidin- | | 7.31-7.26 (m, 1H), 6.97 (td, J = |
| | | 1-yl)-3,3-dimethyl-1- | | 7.8, 7.2, 1.3 Hz, 2H), 4.67-4.52 |
| | | oxobutan-2-yl)amino)-6- | | (m, 4H), 4.35 (d, J = 15.5 Hz, 1H), |
| | | oxohexyl)-3-(2- | | 3.95-3.91 (m, 1H), 3.83-3.78 (m, |
| | | hydroxyphenyl)-6,7,8,9- | | 1H), 3.26 (td, J = 6.9, 2.6 Hz, 2H), |
| | | tetrahydro-5H- | | 3.03-2.83 (m, 4H), 2.75-2.67 (m, |
| | | pyridazino[3,4-b]indole-6- | | 1H), 2.47 (s, 3H), 2.36-2.20 (m, |
| | | carboxamide | | 4H), 2.14-2.02 (m, 2H), 1.73- |
| | | | | 1.54 (m, 4H), 1.46-1.38 (m, 2H), |
| | | | | 1.06 (s, 9H). |
| 190 | A | (S)-N-(4-(((S)-1-((2S,4R)-4- | 807.50 | 1H NMR (300 MHz, Methanol-d4) δ |
| | | hydroxy-2-((4-(4- | | 8.86 (s, 1H), 8.29 (s, 1H), 7.93 (d, |
| | | methylthiazol-5- | | J = 8.0, 1.6 Hz, 1H), 7.48-7.38 (m, |
| | | yl)benzyl)carbamoyl)pyrrolidin- | | 4H), 7.28 (t, J = 7.7 Hz, 1H), 6.99- |
| | | 1-yl)-3,3-dimethyl-1- | | 6.93 (m, 2H), 4.66-4.52 (m, 4H), |
| | | oxobutan-2-yl)amino)-4- | | 4.37-4.32 (m, 1H), 3.97-3.80 (m, |
| | | oxobutyl)-3-(2- | | 2H), 3.30-3.25 (m, 1H), 3.05- |
| | | hydroxyphenyl)-6,7,8,9- | | 2.83 (m, 4H), 2.76-2.68 (m, 1H), |
| | | tetrahydro-5H- | | 2.44 (s, 3H), 2.37 (td, J = 7.3, 1.5 |
| | | pyridazino[3,4-b]indole-6- | | Hz, 2H), 2.27-2.20 (m, 2H), 2.15- |
| | | carboxamide | | 1.99 (m, 2H), 1.91-1.82 (m, 2H), |
| | | | | 1.08 (s, 9H). |
| 154 | A | (2S,4R)-4-hydroxy-1-((S)-2- | 829.30 | 1H NMR (300 MHz, DMSO-d6) δ |
| | | (3-(1-((R)-3-(2- | | 14.41 (s, 1H), 9.04-8.87 (m, 1H), |
| | | hydroxyphenyl)-6,7,8,9- | | 8.52 (d, J = 7.6 Hz, 1H), 8.40-8.24 |
| | | tetrahydro-5H- | | (m, 1H), 8.12-8.00 (m, 1H), 7.54- |
| | | pyridazino[3,4-b]indole-6- | | 7.37 (m, 2H), 7.36-7.17 (m, 3H), |
| | | carbonyl)azetidin-3- | | 7.00-6.87 (m, 2H), 6.60-6.44 (m, |
| | | yl)isoxazol-5-yl)-3- | | 1H), 5.16-4.72 (m, 2H), 4.70- |
| | | methylbutanoyl)-N-((S)-1-(4- | | 4.38 (m, 2H), 4.38-4.19 (m, 3H), |
| | | (4-methylthiazol-5- | | 4.09-3.85 (m, 3H), 3.63-3.42 (m, |
| | | yl)phenyl)ethyl)pyrrolidine-2- | | 2H), 2.92 (s, 3H), 2.81-2.69 (m, |
| | | carboxamide | | 2H), δ 2.47-2.44 (m, 1H), 2.44- |
| | | | | 2.37 (m, 1H), 2.36-2.29 (m, 1H), |
| | | | | 2.29-2.24 (m, 1H), 2.18-1.99 (m, |
| | | | | 2H), 1.99-1.70 (m, 2H), 1.50- |
| | | | | 1.26 (m, 3H), 1.03-0.72 (m, 6H). |
| 170 | A | N1-((S)-1-((2S,4R)-4- | 821.40 | 1H NMR (400 MHz, Methanol-d4) δ |
| | | hydroxy-2-((4-(4- | | 8.84 (s, 1H), 8.27 (s, 1H), 7.92- |
| | | methylthiazol-5- | | 7.82 (m, 1H), 7.44-7.36 (m, 4H), |
| | | yl)benzyl)carbamoyl)pyrrolidin- | | 7.28-7.25 (m, 1H), 6.97-6.93 (m, |
| | | 1-yl)-3,3-dimethyl-1- | | 2H), 4.64-4.48 (m, 4H), 4.35- |
| | | oxobutan-2-yl)-N6-((R)-3-(2- | | 4.24 (m, 2H), 3.90 (d, J = 11.0 Hz, |
| | | hydroxyphenyl)-6,7,8,9- | | 1H), 3.79 (dd, J = 10.9, 4.0 Hz, 1H), |
| | | tetrahydro-5H- | | 3.11 (dd, J = 15.3, 5.4 Hz, 1H), 2.97 |
| | | pyridazino[3,4-b]indol-6- | | (t, J = 6.5 Hz, 2H), 2.65 (dd, J = |
| | | yl)adipamide | | 15.3, 8.2 Hz, 1H), 2.44 (s, 3H), 2.35- |
| | | | | 2.19 (m, 6H), 2.17-1.95 (m, 3H), |
| | | | | 1.66-1.64 (m, 4H), 1.03 (s, 9H). |
| 171 | A | N1-((S)-1-((2S,4R)-4- | 793.60 | 1H NMR (400 MHz, Methanol-d4) δ |
| | | hydroxy-2-((4-(4- | | 8.85 (s, 1H), 8.31 (s, 1H), 7.94 (dd, |
| | | methylthiazol-5- | | J = 7.9, 1.6 Hz, 1H), 7.45-7.37 (m, |
| | | yl)benzyl)carbamoyl)pyrrolidin- | | 4H), 7.32-7.25 (m, 1H), 6.97- |
| | | 1-yl)-3,3-dimethyl-1- | | 6.93 (m, 2H), 4.61 (s, 1H), 4.59- |
| | | oxobutan-2-yl)-N4-((R)-3-(2- | | 4.50 (m, 3H), 4.41-4.32 (m, 1H), |
| | | hydroxyphenyl)-6,7,8,9- | | 4.27-4.24 (m, 1H), 3.90 (d, J = |
| | | tetrahydro-5H- | | 11.1 Hz, 1H), 3.79 (dd, J = 11.0, 3.9 |
| | | pyridazino[3,4-b]indol-6- | | Hz, 1H), 3.13 (dd, J = 15.4, 5.4 Hz, |
| | | yl)succinamide | | 1H), 3.00-2.99 (m, 2H), 2.69- |
| | | | | 2.56 (m, 3H), 2.53-2.49 (m, 2H), |
| | | | | 2.45 (s, 3H), 2.24-1.96 (m, 4H), |
| | | | | 1.04 (s, 9H). |
| 176 | A | (2S,4R)-4-hydroxy-1-((S)-2- | 775.25 | 1H NMR (300 MHz, DMSO-d6) δ |
| | | (3-(1-(3-(2-hydroxyphenyl)- | | 13.47 (s, 1H), 8.98 (d, J = 10.8 Hz, |
| | | 7H-pyrrolo[2,3-c]pyridazine- | | 1H), 8.68 (s, 1H), 8.45-8.20 (m, |
| | | 6-carbonyl)azetidin-3- | | 1H), 8.07-7.93 (m, 1H), 7.53- |
| | | yl)isoxazol-5-yl)-3- | | 7.17 (m, 5H), 6.68-6.54 (m, 3H), |
| | | methylbutanoyl)-N-((S)-1-(4- | | 6.68-6.54 (m, 1H), 5.26-4.72 (m, |
| | | (4-methylthiazol-5- | | 3H), 4.61 (d, J = 39.4 Hz, 2H), 4.49- |
| | | yl)phenyl)ethyl)pyrrolidine-2- | | 4.00 (m, 4H), 3.99-3.80 (m, 1H), |
| | | carboxamide | | 3.64-3.55 (m, 1H), 3.55-3.48 (m, |
| | | | | 1H), 2.47-2.30 (m, 4H), 2.15- |
| | | | | 1.95 (m, 1H), 1.90-1.70 (m, 1H), |
| | | | | 1.57-1.19 (m, 3H), 1.08-0.67 (m, |
| | | | | 6H). |
| 99 | A | (2S,4R)-4-hydroxy-1-((S)-2- | 870.20 | 1H NMR (400 MHz, DMSO-d6) δ |
| | | (3-((1R,4R)-5-((R)-3-(2- | | 14.44 (s, 1H), 12.32 (s, 1H), 9.02- |
| | | hydroxyphenyl)-6,7,8,9- | | 8.81 (m, 1H), 8.57-8.39 (m, 1H), |
| | | tetrahydro-5H- | | 8.32-8.25 (m, 1H), 8.01 (s, 1H), |
| | | pyridazino[3,4-b]indole-6- | | 7.51-7.18 (m, 5H), 7.00-6.91 (m, |
| | | carbonyl)-2,5- | | 2H), 6.15-5.99 (m, 1H), 5.15- |
| | | diazabicyclo[2.2.1]heptan-2- | | 4.93 (m, 1H), 4.91-4.84 (m, 1H), |
| | | yl)isoxazol-5-yl)-3- | | 4.83-4.73 (m, 1H), 4.56-4.23 (m, |
| | | methylbutanoyl)-N-((S)-1-(4- | | 3H), 3.73-3.65 (m, 1H), 3.64- |
| | | (4-methylthiazol-5- | | 3.57 (m, 1H), 3.57-3.41 (m, 2H), |
| | | yl)phenyl)ethyl)pyrrolidine-2- | | 3.40-3.37 (m, 2H), 3.29-3.21 (m, |
| | | carboxamide | | 1H), 3.17-2.98 (m, 2H), 2.97- |
| | | | | 2.87 (m, 1H), 2.84-2.64 (m, 2H), |
| | | | | 2.47-2.31 (m, 3H), 2.31-2.20 (m, |
| | | | | 1H), 2.15-2.02 (m, 1H), 2.00- |
| | | | | 1.74 (m, 4H), 1.72-1.68 (m, 1H), |
| | | | | 1.47 (d, J = 6.9 Hz, 1H), 1.34 (dd, |
| | | | | J = 17.7, 7.0 Hz, 2H), 1.01-0.94 (m, |
| | | | | 2H), 0.87-0.79 (m, 3H), 0.78 (t, |
| | | | | J = 7.1 Hz, 1H). |
| 100 | A | (2S,4R)-4-hydroxy-1-((S)-2- | 870.45 | 1H NMR (300 MHz, DMSO-d6) δ |
| | | (3-((1S,4S)-5-((R)-3-(2- | | 14.51 (s, 1H), 9.06 (m, J = 7.7, 1.6 |
| | | hydroxyphenyl)-6,7,8,9- | | Hz, 1H), 8.57 (d, J = 25.5 Hz, 1H), |
| | | tetrahydro-5H- | | 8.42-8.30 (m, 1H), 8.13 (m, J = |
| | | pyridazino[3,4-b]indole-6- | | 13.8, 7.7 Hz, 1H), 7.60-7.46 (m, |
| | | carbonyl)-2,5- | | 2H), 7.42 (q, J = 4.1 Hz, 1H), 7.34 |
| | | diazabicyclo[2.2.1]heptan-2- | | (d, J = 8.0 Hz, 1H), 7.28 (d, J = 8.3 |
| | | yl)isoxazol-5-yl)-3- | | Hz, 1H), 7.00 (m, J = 13.8, 7.4 Hz, |
| | | methylbutanoyl)-N-((S)-1-(4- | | 2H), 6.18 (d, J = 4.5 Hz, 1H), 5.19 |
| | | (4-methylthiazol-5- | | (s, 1H), 4.90 (d, J = 17.3 Hz, 2H), |
| | | yl)phenyl)ethyl)pyrrolidine-2- | | 4.57-4.40 (m, 2H), 4.34 (s, 1H), |
| | | carboxamide | | 3.75 (dt, J = 16.0, 8.1 Hz, 1H), 3.65- |
| | | | | 3.47 (m, 4H), 3.37-3.16 (m, 2H), |
| | | | | 3.08 (d, J = 15.3 Hz, 1H), 2.98 (s, |
| | | | | 1H), 2.89-2.72 (m, 2H), 2.57- |
| | | | | 2.46 (m, 3H), 2.41-2.21 (m, 1H), |
| | | | | 2.12 (d, J = 16.2 Hz, 2H), 2.03- |
| | | | | 1.75 (m, 4H), 1.53 (t, J = 6.2 Hz, |
| | | | | 1H), 1.43 (d, J = 7.0 Hz, 1H), 1.34 |
| | | | | (d, J = 7.0 Hz, 1H), 1.02 (m, J = |
| | | | | 15.4, 6.5 Hz, 2H), 0.91-0.84 (m, |
| | | | | J = 14.7, 6.4 Hz, 4H). |
| 116 | B | (2S,4R)-1-((R)-2-(3-(2-(3-(7- | 841.20 | 1H NMR (400 MHz, DMSO-d6) δ |
| | | (difluoromethyl)-3-(2- | | 12.91 (s, 1H), 8.99 (s, 1H), 8.90 (s, |
| | | hydroxyphenyl)-7H- | | 1H), 8.52-8.16 (m, 3H), 8.06 (dd, |
| | | pyrrolo[2,3-c]pyridazin-5- | | J = 7.9, 1.7 Hz, 1H), 7.48-7.40 (m, |
| | | yl)azetidin-1- | | 2H), 7.40-7.30 (m, 3H), 7.06- |
| | | yl)ethoxy)isoxazol-5-yl)-3- | | 6.96 (m, 2H), 6.09 (s, 1H), 5.10 (d, |
| | | methylbutanoyl)-4-hydroxy- | | J = 3.6 Hz, 1H), 4.96-4.85 (m, |
| | | N-((S)-1-(4-(4-methylthiazol- | | 1H), 4.36 (t, J = 7.9 Hz, 1H), 4.31- |
| | | 5-yl)phenyl)ethyl)pyrrolidine- | | 4.22 (m, 3H), 4.08-3.84 (m, 3H), |
| | | 2-carboxamide | | 3.74-3.62 (m, 2H), 3.60-3.48 (m, |
| | | | | 1H), 3.48-3.39 (m, 2H), 3.06- |
| | | | | 3.02 (m, 2H), 2.45 (s, 3H), 2.32- |
| | | | | 2.14 (m, 1H), 2.05-2.00 (m, 1H), |
| | | | | 1.83-1.73 (m, 1H), 1.48-1.34 (m, |
| | | | | 3H), 1.00-0.92 (m, 3H), 0.85- |
| | | | | 0.75 (m, 3H). |
| 153 | A | (2S,4R)-4-hydroxy-1-((S)-2- | 829.65 | 1H NMR (300 MHz, Methanol-d4) δ |
| | | (3-(1-((S)-3-(2- | | 8.84 (d, J = 3.6 Hz, 1H), 8.36 (d, J = |
| | | hydroxyphenyl)-6,7,8,9- | | 5.0 Hz, 1H), 7.97 (t, J = 7.4 Hz, 1H), |
| | | tetrahydro-5H- | | 7.52-7.20 (m, 5H), 7.04-6.90 (m, |
| | | pyridazino[3,4-b]indole-6- | | 2H), 6.49 (d, J = 3.6 Hz, 1H), 5.04- |
| | | carbonyl)azetidin-3- | | 4.92 (m, 1H), 4.74 (t, J = 8.7 Hz, |
| | | yl)isoxazol-5-yl)-3- | | 1H), 4.61 (t, J = 8.1 Hz, 1H), 4.53- |
| | | methylbutanoyl)-N-((S)-1-(4- | | 4.36 (m, 3H), 4.26-4.11 (m, 1H), |
| | | (4-methylthiazol-5- | | 4.09-4.98 (m, 1H), 3.95 (dd, J = |
| | | yl)phenyl)ethyl)pyrrolidine-2- | | 9.2, 2.1 Hz, 1H), 3.78-3.58 (m, |
| | | carboxamide | | 2H), 3.06-2.94 (m, 3H), 2.92- |
| | | | | 2.81 (m, 2H), 2.54-2.38 (m, 4H), |
| | | | | 2.29-2.15 (m, 2H), 2.14-1.89 (m, |
| | | | | 2H), 1.65-1.40 (m, 3H), 1.05 (dd, |
| | | | | J = 31.2, 6.6 Hz, 3H), 0.91 (dd, J = |
| | | | | 20.2, 6.7 Hz, 3H). |
| 89 | A | (6R)-6-(2-((5-(1-((2S,4R)-4- | 863.45 | 1H NMR (400 MHz, DMSO-d6) δ |
| | | hydroxy-2-(((S)-1-(4-(4- | | 8.98 (d, J = 5.5 Hz, 1H), 8.53 (s, |
| | | methylthiazol-5- | | 1H), 8.44 (d, J = 7.7 Hz, 1H), 8.37- |
| | | yl)phenyl)ethyl)carbamoyl)pyrrolidin- | | 8.22 (m, 1H), 8.10 (d, J = 7.8 Hz, |
| | | 1-yl)-3-methyl-1- | | 1H), 7.48-7.31 (m, 4H), 7.30- |
| | | oxobutan-2-yl)isoxazol-3- | | 7.23 (m, 1H), 6.99-6.89 (m, 2H), |
| | | yl)oxy)acetamido)-3-(2- | | 6.20 (s, 1H), 4.96-4.84 (m, 1H), |
| | | hydroxyphenyl)-6,7,8,9- | | 4.64-4.49 (m, 3H), 4.45-4.33 (m, |
| | | tetrahydro-5H- | | 1H), 4.32-4.22 (m, 1H), 3.77- |
| | | pyridazino[3,4-b]indole-6- | | 3.61 (m, 2H), 3.61-3.50 (m, 1H), |
| | | carboxylic acid | | 3.26-3.15 (m, 1H), 3.07-2.87 (m, |
| | | | | 3H), 2.87-2.73 (m, 1H), 2.45 (d, |
| | | | | J = 4.6 Hz, 3H), 2.31-2.13 (m, 1H), |
| | | | | 2.11-1.86 (m, 2H), 1.86-1.70 (m, |
| | | | | 1H), 1.36 (d, J = 7.0 Hz, 3H), 0.95 |
| | | | | (d, J = 6.6 Hz, 3H), 0.80 (d, J = 6.6 |
| | | | | Hz, 3H). |
| 88 | A | (6S)-6-(2-((5-(1-((2S,4R)-4- | 863.45 | 1H NMR (400 MHz, DMSO-d6) δ |
| | | hydroxy-2-(((S)-1-(4-(4- | | 12.41-12.06 (m, 1H), 8.98 (d, J = |
| | | methylthiazol-5- | | 4.3 Hz, 1H), 8.57 (s, 1H), 8.47- |
| | | yl)phenyl)ethyl)carbamoyl)pyrrolidin- | | 8.29 (m, 2H), 8.11 (d, J = 7.8 Hz, |
| | | 1-yl)-3-methyl-1- | | 1H), 7.48-7.23 (m, 5H), 6.99- |
| | | oxobutan-2-yl)isoxazol-3- | | 6.89 (m, 2H), 6.12 (d, J = 13.3 Hz, |
| | | yl)oxy)acetamido)-3-(2- | | 1H), 5.19-4.79 (m, 2H), 4.69- |
| | | hydroxyphenyl)-6,7,8,9- | | 4.49 (m, 2H), 4.45-4.32 (m, 1H), |
| | | tetrahydro-5H- | | 4.31-4.20 (m, 1H), 3.77-3.60 (m, |
| | | pyridazino[3,4-b]indole-6- | | 2H), 3.58-3.40 (m, 1H), 3.27- |
| | | carboxylic acid | | 3.14 (m, 1H), 3.11-2.99 (m, 1H), |
| | | | | 2.97-2.76 (m, 2H), 2.69-2.56 (m, |
| | | | | 1H), 2.45 (d, J = 3.9 Hz, 3H), 2.28- |
| | | | | 2.15 (m, 1H), 2.14-1.97 (m, 2H), |
| | | | | 1.86-1.69 (m, 1H), 1.37 (d, J = 7.0 |
| | | | | Hz, 3H), 0.95 (d, J = 6.6 Hz, 3H), |
| | | | | 0.78 (d, J = 6.6 Hz, 3H). |
| 91 | A | (6R)-6-((2-((5-(1-((2S,4R)-4- | 849.45 | 1H NMR (400 MHz, DMSO-d6) δ |
| | | hydroxy-2-(((S)-1-(4-(4- | | 12.33-12.09 (m, 1H), 8.98 (d, J = |
| | | methylthiazol-5- | | 6.3 Hz, 1H), 8.53 (s, 1H), 8.45- |
| | | yl)phenyl)ethyl)carbamoyl)pyrrolidin- | | 8.28 (m, 1H), 8.10 (d, J = 8.0 Hz, |
| | | 1-yl)-3-methyl-1- | | 1H), 7.49-7.23 (m, 6H), 6.99- |
| | | oxobutan-2-yl)isoxazol-3- | | 6.89 (m, 2H), 6.05 (d, J = 7.3 Hz, |
| | | yl)oxy)ethyl)amino)-3-(2- | | 1H), 4.96-4.79 (m, 1H), 4.45- |
| | | hydroxyphenyl)-6,7,8,9- | | 4.09 (m, 5H), 3.76-3.42 (m, 4H), |
| | | tetrahydro-5H- | | 3.22-3.02 (m, 1H), 2.98-2.69 (m, |
| | | pyridazino[3,4-b]indole-6- | | 5H), 2.44 (d, J = 7.4 Hz, 3H), 2.27- |
| | | carboxylic acid | | 2.08 (m, 2H), 2.07-1.95 (m, 1H), |
| | | | | 1.81-1.70 (m, 1H), 1.35 (d, J = 7.0 |
| | | | | Hz, 3H), 0.94 (d, J = 6.6 Hz, 3H), |
| | | | | 0.76 (d, J = 6.6 Hz, 3H). |
| 93 | A | (2S,4R)-4-hydroxy-1-((S)-2- | 911.30 | 1H NMR (300 MHz, DMSO-d6) δ |
| | | ((1r,4S)-4-(2-(4-(3-(2- | | 14.18 (br s, 1H), 12.54 (s, 1H), 9.00 |
| | | hydroxyphenyl)-7H- | | (s, 1H), 8.62-8.52 (m, 2H), 8.29 |
| | | pyrrolo[2,3-c]pyridazin-6- | | (s, 2H), 8.07-7.98 (m, 1H), 7.80 |
| | | yl)piperidin-1-yl)pyrimidin-5- | | (d, J = 9.2 Hz, 1H), 7.48-7.35 (m, |
| | | yl)cyclohexane-1- | | 4H), 7.35-7.23 (m, 1H), 7.00- |
| | | carboxamido)-3,3- | | 6.91 (m, 2H), 6.46 (s, 1H), 5.15 (d, |
| | | dimethylbutanoyl)-N-(4-(4- | | J = 3.5 Hz, 1H), 4.79 (d, J = 13.1 |
| | | methylthiazol-5- | | Hz, 2H), 4.54 (d, J = 9.2 Hz, 1H), |
| | | yl)benzyl)pyrrolidine-2- | | 4.50-4.34 (m, 3H), 4.28-4.17 (m, |
| | | carboxamide | | 1H), 3.74-3.61 (m, 2H), 3.27- |
| | | | | 3.14 (m, 1H), 3.03 (t, J = 12.5 Hz, |
| | | | | 2H), 2.46 (s, 3H), 2.43-2.31 (m, |
| | | | | 2H), 2.19-1.98 (m, 3H), 1.87- |
| | | | | 1.64 (m, 7H), 1.57-1.37 (m, 4H), |
| | | | | 0.95 (s, 9H). |
| 94 | A | (2S,4R)-4-hydroxy-1-((S)-2- | 911.30 | 1H NMR (300 MHz, DMSO-d6) δ |
| | | ((1s,4R)-4-(2-(4-(3-(2- | | 12.54 (br s, 1H), 8.99 (s, 1H), 8.61- |
| | | hydroxyphenyl)-7H- | | 8.52 (m, 2H), 8.30-8.23 (m, 2H), |
| | | pyrrolo[2,3-c]pyridazin-6- | | 7.96 (s, 1H), 7.70 (d, J = 9.3 Hz, |
| | | yl)piperidin-1-yl)pyrimidin-5- | | 1H), 7.47-7.36 (m, 4H), 7.32 (t, |
| | | yl)cyclohexane-1- | | J = 7.8 Hz, 1H), 7.03-6.93 (m, 2H), |
| | | carboxamido)-3,3- | | 6.56-6.49 (s, 1H), 5.14 (s, 1H), |
| | | dimethylbutanoyl)-N-(4-(4- | | 4.79 (d, J = 13.1 Hz, 2H), 4.58 (d, |
| | | methylthiazol-5- | | J = 9.4 Hz, 1H), 4.50-4.32 (m, 3H), |
| | | yl)benzyl)pyrrolidine-2- | | 4.28-4.17 (m, 1H), 3.68 (s, 2H), |
| | | carboxamide | | 3.28-3.15 (m, 1H), 3.04 (t, J = |
| | | | | 12.4 Hz, 2H), 2.69-2.61 (m, 2H), |
| | | | | 2.45 (s, 3H), 2.17-2.00 (m, 3H), |
| | | | | 1.98-1.65 (m, 9H), 1.64-1.39 (m, |
| | | | | 2H), 0.96 (s, 9H). |
| 110 | B | (2S,4R)-1-((R)-2-(3-(2- | 851.40 | 1H NMR (300 MHz, Methanol-d4) δ |
| | | ((1R,5S,6R)-6-(3-(3-chloro- | | 8.89 (s, 1H), 8.37 (s, 1H), 7.95- |
| | | 2-hydroxyphenyl)-7H- | | 7.80 (m, 1H), 7.48-7.42 (m, 3H), |
| | | pyrrolo[2,3-c]pyridazin-6-yl)- | | 7.39-7.24 (m, 2H), 6.93 (t, J = 7.7 |
| | | 3-azabicyclo[3.1.0]hexan-3- | | Hz, 1H), 6.30 (s, 1H), 6.06 (s, 1H), |
| | | yl)ethoxy)isoxazol-5-yl)-3- | | 5.26-4.93 (m, 2H), 4.56 (t, J = 8.2 |
| | | methylbutanoyl)-4-hydroxy- | | Hz, 1H), 4.49-4.43 (m, 1H), 4.37- |
| | | N-((S)-1-(4-(4-methylthiazol- | | 4.29 (m, 2H), 3.92-3.81 (m, 1H), |
| | | 5-yl)phenyl)ethyl)pyrrolidine- | | 3.79-3.38 (m, 3H), 2.95 (t, J = 5.5 |
| | | 2-carboxamide | | Hz, 2H), 2.66 (d, J = 9.0 Hz, 2H), |
| | | | | 2.57-2.51 (m, 1H), 2.50-2.47 (m, |
| | | | | 2H), 2.46-2.36 (m, 2H), 2.28- |
| | | | | 2.13 (m, 1H), 2.11-2.05 (m, 2H), |
| | | | | 2.04-1.91 (m, 1H), 1.54 (d, J = 7.0 |
| | | | | Hz, 3H), 1.08 (d, J = 6.6 Hz, 3H), |
| | | | | 0.93 (d, J = 6.5 Hz, 3H). |
| 132 | B | (2S,4R)-4-hydroxy-1-((R)-2- | 845.20 | 1H NMR (300 MHz, DMSO-d6) δ |
| | | (3-(2-(((1S,4s,6S)-6-(3-(2- | | 14.36 (s, 1H), 9.06 (s, 1H), 8.59 (d, |
| | | hydroxyphenyl)-7H- | | J = 2.8 Hz, 1H), 8.48 (d, J = 7.6 Hz, |
| | | pyrrolo[2,3-c]pyridazin-6- | | 1H), 8.10 (d, J = 7.6 Hz, 1H), 7.55- |
| | | yl)spiro[3.3]heptan-1- | | 7.48 (m, 2H), 7.47-7.40 (m, 2H), |
| | | yl)amino)ethoxy)isoxazol-5- | | 7.39-7.28 (m, 1H), 7.02 (t, J = 7.5 |
| | | yl)-3-methylbutanoyl)-N-((S)- | | Hz, 2H), 6.49 (s, 1H), 6.15 (d, J = |
| | | 1-(4-(4-methylthiazol-5- | | 1.3 Hz, 1H), 5.17 (d, J = 3.6 Hz, |
| | | yl)phenyl)ethyl)pyrrolidine-2- | | 1H), 4.98 (t, J = 7.2 Hz, 1H), 4.44 (t, |
| | | carboxamide | | J = 7.9 Hz, 1H), 4.51-4.41 (m, |
| | | | | 3H), 3.85-3.64 (m, 3H), 3.72- |
| | | | | 3.67 (m, 1H), 3.10-2.96 (m, 3H), |
| | | | | 2.82-2.67 (m, 1H), 2.68 (t, J = |
| | | | | 10.0 Hz, 3H), 2.58-2.42 (m, 1H), |
| | | | | 2.39-2.22 (m, 4H), 2.18-2.05 (m, |
| | | | | 2H), 2.02-1.93 (m, 1H), 1.92- |
| | | | | 1.72 (m, 2H), 1.68-1.55 (m, 1H), |
| | | | | 1.53-1.40 (m, 3H), 1.01 (d, J = 6.6 |
| | | | | Hz, 3H), 0.83 (d, J = 6.9 Hz, 3H). |
| 133 | B | (2S,4R)-4-hydroxy-1-((R)-2- | 845.20 | 1H NMR (300 MHz, DMSO-d6) δ |
| | | (3-(2-(((1S,4r,6R)-6-(3-(2- | | 14.33 (s, 1H), 12.54 (s, 1H), 9.06 (s, |
| | | hydroxyphenyl)-7H- | | 1H), 8.59 (s, 1H), 8.50 (d, J = 7.6 |
| | | pyrrolo[2,3-c]pyridazin-6- | | Hz, 1H), 8.10 (d, J = 7.7 Hz, 1H), |
| | | yl)spiro[3.3]heptan-1- | | 7.60-7.49 (m, 2H), 7.58-7.40 (m, |
| | | yl)amino)ethoxy)isoxazol-5- | | 2H), 7.39-7.28 (m, 1H), 7.03 (d, |
| | | yl)-3-methylbutanoyl)-N-((S)- | | J = 7.6 Hz, 2H), 6.53 (s, 1H), 6.19 (s, |
| | | 1-(4-(4-methylthiazol-5- | | 1H), 5.19 (d, J = 3.6 Hz, 1H), 4.98 |
| | | yl)phenyl)ethyl)pyrrolidine-2- | | (t, J = 7.1 Hz, 1H), 4.45 (t, J = 7.8 |
| | | carboxamide | | Hz, 1H), 4.41-4.23 (m, 3H), 3.88- |
| | | | | 3.62 (m, 3H), 3.56-3.50 (m, 1H), |
| | | | | 3.18-3.12 (m, 1H), 3.10-3.05 (m, |
| | | | | 2H), 3.02-2.94 (m, 1H), 2.55- |
| | | | | 2.52 (m, 4H), 2.38-2.25 (m, 3H), |
| | | | | 2.22-2.05 (m, 3H), 1.91-1.78 (m, |
| | | | | 1H), 1.78-1.60 (m, 2H), 1.55- |
| | | | | 1.50 (m, 1H), 1.49 (dd, J = 21.8, 7.7 |
| | | | | Hz, 3H), 1.03 (d, J = 6.5 Hz, 3H), |
| | | | | 0.87 (d, J = 6.8 Hz, 3H). |
| 141 | B | (2S,4R)-1-((R)-2-(3-(2- | 853.50 | 1H NMR (300 MHz, Methanol-d4) δ |
| | | ((1R,5S,6R)-6-(3-(3,5- | | 8.83 (s, 1H), 8.28 (s, 1H), 7.63- |
| | | difluoro-2-hydroxyphenyl)- | | 7.53 (m, 1H), 7.52-7.39 (m, 3H), |
| | | 7H-pyrrolo[2,3-c]pyridazin-6- | | 7.34-7.24 (m, 1H), 7.10-6.96 (m, |
| | | yl)-3-azabicyclo[3.1.0]hexan- | | 1H), 6.27 (s, 1H), 6.01 (s, 1H), 5.12- |
| | | 3-yl)ethoxy)isoxazol-5-yl)-3- | | 4.96 (m, 1H), 4.56 (t, J = 8.2 Hz, |
| | | methylbutanoyl)-4-hydroxy- | | 1H), 4.50-4.43 (m, 1H), 4.34 (t, J = |
| | | N-((S)-1-(4-(4-methylthiazol- | | 5.5 Hz, 2H), 3.90-3.83 (m, 1H), |
| | | 5-yl)phenyl)ethyl)pyrrolidine- | | 3.83-3.39 (m, 3H), 2.95 (t, J = 5.4 |
| | | 2-carboxamide | | Hz, 2H), 2.67 (d, J = 9.0 Hz, 2H), |
| | | | | 2.58-2.52 (m, 1H), 2.50 (s, 3H), |
| | | | | 2.46-2.35 (m, 2H), 2.29-2.15 (m, |
| | | | | 1H), 2.12-2.06 (m, 2H), 2.05- |
| | | | | 1.91 (m, 1H), 1.54 (d, J = 7.0 Hz, |
| | | | | 3H), 1.08 (d, J = 6.5 Hz, 3H), 0.93 |
| | | | | (d, J = 6.5 Hz, 3H). |
| 142 | B | (2S,4R)-1-((R)-2-(3-(2- | 835.55 | 1H NMR (300 MHz, Methanol-d4) δ |
| | | ((1R,5S,6R)-6-(3-(3-fluoro-2- | | 8.89 (s, 1H), 8.35 (s, 1H), 7.75 (d, |
| | | hydroxyphenyl)-7H- | | J = 8.2 Hz, 1H), 7.51-7.38 (m, 3H), |
| | | pyrrolo[2,3-c]pyridazin-6-yl)- | | 7.35-7.23 (m, 1H), 7.22-7.07 (m, |
| | | 3-azabicyclo[3.1.0]hexan-3- | | 1H), 6.98-6.85 (m, 1H), 6.26 (s, |
| | | yl)ethoxy)isoxazol-5-yl)-3- | | 1H), 6.01 (s, 1H), 5.11-4.99 (m, |
| | | methylbutanoyl)-4-hydroxy- | | 1H), 4.66-4.51 (m, 1H), 4.50- |
| | | N-((S)-1-(4-(4-methylthiazol- | | 4.43 (m, 1H), 4.34 (t, J = 5.4 Hz, |
| | | 5-yl)phenyl)ethyl)pyrrolidine- | | 2H), 3.93-3.77 (m, 1H), 3.77- |
| | | 2-carboxamide | | 3.46 (m, 3H), 2.95 (t, J = 5.5 Hz, |
| | | | | 2H), 2.67 (d, J = 8.9 Hz, 2H), 2.57- |
| | | | | 2.52 (m, 1H), 2.50 (s, 3H), 2.47- |
| | | | | 2.35 (m, 2H), 2.29-2.14 (m, 1H), |
| | | | | 2.13-2.05 (m, 2H), 2.06-1.92 (m, |
| | | | | 1H), 1.54 (d, J = 7.0 Hz, 3H), 1.08 |
| | | | | (d, J = 6.5 Hz, 3H), 0.93 (d, J = 6.5 |
| | | | | Hz, 3H). |
| 161 | B | (2S,4R)-4-hydroxy-1-((R)-2- | 831.20 | 1H NMR (400 MHz, DMSO-d6) δ |
| | | (3-(2-(6-(3-(2- | | 14.07 (s, 1H), 12.59 (d, J = 40.1 Hz, |
| | | hydroxyphenyl)-7H- | | 1H), 8.99 (s, 1H), 8.56 (d, J = 11.1 |
| | | pyrrolo[2,3-c]pyridazin-6-yl)- | | Hz, 1H), 8.40 (d, J = 7.7 Hz, 1H), |
| | | 1-azaspiro[3.3]heptan-1- | | 8.03 (d, J = 8.0 Hz, 1H), 7.44 (d, J = |
| | | yl)ethoxy)isoxazol-5-yl)-3- | | 8.0 Hz, 2H), 7.36 (dd, J = 8.4, 2.6 |
| | | methylbutanoyl)-N-((S)-1-(4- | | Hz, 2H), 7.33-7.25 (m, 1H), 6.96 |
| | | (4-methylthiazol-5- | | (t, J = 7.9 Hz, 2H), 6.52 (s, 1H), |
| | | yl)phenyl)ethyl)pyrrolidine-2- | | 6.21-5.98 (m, 1H), 5.12 (s, 1H), |
| | | carboxamide | | 4.95-4.87 (m, 1H), 4.46-4.24 (m, |
| | | | | 4H), 4.20-3.81 (m, 2H), 3.73- |
| | | | | 3.69 (m, 3H), 3.49 (d, J = 15.0 Hz, |
| | | | | 2H), 2.81-2.70 (m, 1H), 2.46 (s, |
| | | | | 5H), 2.33-2.18 (m, 2H), 2.03 (s, |
| | | | | 1H), 1.79 (d, J = 3.8 Hz, 1H), 1.42 |
| | | | | (d, J = 6.5 Hz, 3H), 0.96 (d, J = 6.4 |
| | | | | Hz, 3H), 0.94-0.76 (m, 3H). |
| 163 | B | (2S,4R)-4-hydroxy-1-((R)-2- | 816.25 | 1H NMR (400 MHz, DMSO-d6) δ |
| | | (3-(3-((3-(3-(2- | | 14.14 (s, 1H), 12.47 (s, 1H), 8.98 (s, |
| | | hydroxyphenyl)-7H- | | 1H), 8.55 (s, 1H), 8.38 (d, J = 7.6 |
| | | pyrrolo[2,3-c]pyridazin-6- | | Hz, 1H), 8.06-8.00 (m, 1H), 7.49- |
| | | yl)azetidin-1- | | 7.40 (m, 2H), 7.40-7.33 (m, 2H), |
| | | yl)methyl)azetidin-1- | | 7.33-7.24 (m, 1H), 7.00-6.91 (m, |
| | | yl)isoxazol-5-yl)-3- | | 2H), 6.56 (s, 1H), 5.84 (s, 1H), 5.12- |
| | | methylbutanoyl)-N-((S)-1-(4- | | 5.07 (m, 1H), 4.91 (q, J = 7.2 Hz, |
| | | (4-methylthiazol-5- | | 1H), 4.36 (t, J = 7.9 Hz, 1H), 4.28 |
| | | yl)phenyl)ethyl)pyrrolidine-2- | | (s, 1H), 3.95-3.82 (m, 3H), 3.75- |
| | | carboxamide | | 3.63 (m, 3H), 3.61-3.53 (m, 3H), |
| | | | | 3.45-3.38 (m, 1H), 3.37-3.31 (m, |
| | | | | 2H), 2.73 (s, 3H), 2.46 (s, 3H), 2.26- |
| | | | | 2.14 (m, 1H), 2.06-1.97 (m, 1H), |
| | | | | 1.84-1.73 (m, 1H), 1.42 (dd, J = |
| | | | | 29.0, 6.9 Hz, 3H), 0.99-0.92 (m, |
| | | | | 3H), 0.80 (dd, J = 14.1, 6.7 Hz, 3H). |
| 223 | B | (2S,4R)-4-hydroxy-1-((R)-2- | 845.40 | 1H NMR (300 MHz, DMSO-d6) δ |
| | | (3-(2-((6-(3-(2- | | 14.25 (s, 1H), 12.46 (s, 1H), 8.99 (s, |
| | | hydroxyphenyl)-7H- | | 1H), 8.51 (d, J = 2.7 Hz, 1H), 8.42 |
| | | pyrrolo[2,3-c]pyridazin-6- | | (d, J = 7.6 Hz, 1H), 8.03 (d, J = 7.7 |
| | | yl)spiro[3.3]heptan-2- | | Hz, 1H), 7.49-7.41 (m, 2H), 7.40- |
| | | yl)amino)ethoxy)isoxazol-5- | | 7.34 (m, 2H), 7.33-7.25 (m, 1H), |
| | | yl)-3-methylbutanoyl)-N-((S)- | | 7.00-6.90 (m, 2H), 6.43 (s, 1H), |
| | | 1-(4-(4-methylthiazol-5- | | 6.08 (s, 1H), 5.11 (s, 1H), 4.92 (t, |
| | | yl)phenyl)ethyl)pyrrolidine-2- | | J = 7.2 Hz, 1H), 4.38 (t, J = 7.8 Hz, |
| | | carboxamide | | 1H), 4.29 (s, 1H), 4.15 (t, J = 5.7 |
| | | | | Hz, 2H), 3.79-3.53 (m, 4H), 3.50- |
| | | | | 3.39 (m, 2H), 3.21-3.02 (m, 1H), |
| | | | | 2.79 (t, J = 5.6 Hz, 2H), 2.46 (s, |
| | | | | 3H), 2.40-2.30 (m, 2H), 2.25- |
| | | | | 2.13 (m, 2H), 2.11-1.90 (m, 2H), |
| | | | | 1.88-1.63 (m, 3H), 1.42 (dd, J = |
| | | | | 23.2, 6.9 Hz, 3H), 0.97 (dd, J = 6.7, |
| | | | | 4.2 Hz, 3H), 0.89-0.76 (m, 3H). |
| 224 | B | (2S,4R)-1-((R)-2-(3-(2- | 835.00 | 1H NMR (300 MHz, DMSO-d6) δ |
| | | ((1R,5S,6R)-6-(3-(5-fluoro-2- | | 14.16 (s, 1H), 12.53 (s, 1H), 9.05 |
| | | hydroxyphenyl)-7H- | | (d, J = 4.9 Hz, 1H), 8.59-8.42 (m, |
| | | pyrrolo[2,3-c]pyridazin-6-yl)- | | 2H), 7.97 (dd, J = 10.6, 3.0 Hz, 1H), |
| | | 3-azabicyclo[3.1.0]hexan-3- | | 7.51 (d, J = 8.3 Hz, 2H), 7.44 (d, J = |
| | | yl)ethoxy)isoxazol-5-yl)-3- | | 8.2 Hz, 2H), 7.25-7.12 (m, 1H), |
| | | methylbutanoyl)-4-hydroxy- | | 7.03 (dd, J = 9.0, 5.1 Hz, 1H), 6.39 |
| | | N-((S)-1-(4-(4-methylthiazol- | | (s, 1H), 6.11 (s, 1H), 5.13 (dd, J = |
| | | 5-yl)phenyl)ethyl)pyrrolidine- | | 27.1, 3.3 Hz, 1H), 5.07-4.95 (m, |
| | | 2-carboxamide | | 1H), 4.52-4.21 (m, 4H), 3.85- |
| | | | | 3.70 (m, 2H), 3.60-3.48 (m, 1H), |
| | | | | 3.47-3.42 (m, 1H), 3.30 (d, J = 9.1 |
| | | | | Hz, 2H), 3.01-2.89 (s, 2H), 2.75- |
| | | | | 2.69 (m, 3H), 2.55-2.50 (m, 3H), |
| | | | | 2.33-2.23 (m, 1H), 2.22-2.02 (m, |
| | | | | 3H), 1.92-1.81 (m, 1H), 1.50 (dd, |
| | | | | J = 24.4, 7.0 Hz, 3H), 1.10-0.99 |
| | | | | (m, 3H), 0.97-0.82 (m, 3H). |
| 225 | B | (2S,4R)-1-((R)-2-(3-(2- | 869.30 | 1H NMR (300 MHz, DMSO-d6) δ |
| | | ((1R,5S,6R)-6-(3-(3-chloro- | | 15.71 (s, 1H), 12.61 (s, 1H), 8.98 |
| | | 5-fluoro-2-hydroxyphenyl)- | | (d, J = 5.6 Hz, 1H), 8.64-8.55 (m, |
| | | 7H-pyrrolo[2,3-c]pyridazin-6- | | 1H), 8.42 (d, J = 7.7 Hz, 1H), 8.01 |
| | | yl)-3-azabicyclo[3.1.0]hexan- | | (dd, J = 10.4, 3.0 Hz, 1H), 7.50- |
| | | 3-yl)ethoxy)isoxazol-5-yl)-3- | | 7.42 (m, 3H), 7.37 (d, J = 8.2 Hz, |
| | | methylbutanoyl)-4-hydroxy- | | 2H), 6.37 (d, J = 1.6 Hz, 1H), 6.13 |
| | | N-((S)-1-(4-(4-methylthiazol- | | (s, 1H), 5.12 (d, J = 3.6 Hz, 1H), |
| | | 5-yl)phenyl)ethyl)pyrrolidine- | | 5.06-4.64 (m, 1H), 4.38 (t, J = 7.9 |
| | | 2-carboxamide | | Hz, 1H), 4.34-4.20 (m, 3H), 3.76- |
| | | | | 3.54 (m, 2H), 3.47 (d, J = 10.1 Hz, |
| | | | | 1H), 3.24 (d, J = 9.1 Hz, 2H), 2.94- |
| | | | | 2.85 (m, 2H), 2.65-2.58 (m, 2H), |
| | | | | 2.46 (s, 3H), 2.44 (s, 1H), 2.28- |
| | | | | 2.18 (m, 1H), 2.13 (s, 2H), 2.09- |
| | | | | 1.97 (m, 1H), 1.84-1.71 (m, 1H), |
| | | | | 1.43 (dd, J = 24.7, 6.9 Hz, 3H), |
| | | | | 1.04-0.91 (m, 3H), 0.91-0.78 (m, 3H). |
| 247 | B | (2S,4R)-4-hydroxy-1-((R)-2- | 844.60 | 1H NMR (400 MHz, DMSO-d6) δ |
| | | (3-(4-((3-(3-(2- | | 14.30-14.13 (m, 1H), 8.99 (d, J = |
| | | hydroxyphenyl)-7H- | | 2.0 Hz, 1H), 8.55 (s, 1H), 8.40 (d, |
| | | pyrrolo[2,3-c]pyridazin-6- | | J = 7.7 Hz, 1H), 8.03 (dd, J = 8.1, 1.7 |
| | | yl)azetidin-1- | | Hz, 1H), 7.48-7.41 (m, 2H), 7.41- |
| | | yl)methyl)piperidin-1- | | 7.33 (m, 2H), 7.33-7.24 (m, 1H), |
| | | yl)isoxazol-5-yl)-3- | | 7.00-6.91 (m, 2H), 6.56 (s, 1H), |
| | | methylbutanoyl)-N-((S)-1-(4- | | 6.12 (s, 1H), 5.14-5.07 (m, 1H), |
| | | (4-methylthiazol-5- | | 4.96-4.85 (m, 1H), 4.36 (t, J = 7.8 |
| | | yl)phenyl)ethyl)pyrrolidine-2- | | Hz, 1H), 4.32-4.24 (m, 1H), 3.91- |
| | | carboxamide | | 3.79 (m, 1H), 3.76-3.49 (m, 6H), |
| | | | | 3.47-3.39 (m, 1H), 3.30-3.26 (m, |
| | | | | 2H), 2.80-2.69 (m, 2H), 2.46 (s, |
| | | | | 3H), 2.38 (d, J = 6.8 Hz, 2H), 2.28- |
| | | | | 2.13 (m, 1H), 2.06-1.96 (m, 1H), |
| | | | | 1.84-1.68 (m, 3H), 1.54-1.42 (m, |
| | | | | 1H), 1.38 (d, J = 7.0 Hz, 3H), 1.26- |
| | | | | 1.10 (m, 2H), 0.96 (t, J = 6.7 Hz, |
| | | | | 3H), 0.81 (d, J = 6.7 Hz, 3H). |
| 248 | B | (2S,4R)-4-hydroxy-1-((R)-2- | 830.50 | 1H NMR (400 MHz, DMSO-d6) δ |
| | | (3-(4-(3-(3-(2- | | 14.20-14.14 (m, 1H), 12.67- |
| | | hydroxyphenyl)-7H- | | 12.44 (m, 1H), 8.99 (d, J = 2.1 Hz, |
| | | pyrrolo[2,3-c]pyridazin-6- | | 1H), 8.56 (s, 1H), 8.40 (d, J = 7.7 |
| | | yl)azetidin-1-yl)piperidin-1- | | Hz, 1H), 8.03 (dd, J = 8.0, 1.6 Hz, |
| | | yl)isoxazol-5-yl)-3- | | 1H), 7.48-7.25 (m, 5H), 6.99- |
| | | methylbutanoyl)-N-((S)-1-(4- | | 6.92 (m, 2H), 6.57 (s, 1H), 6.14 (s, |
| | | (4-methylthiazol-5- | | 1H), 5.13-5.07 (m, 1H), 5.02- |
| | | yl)phenyl)ethyl)pyrrolidine-2- | | 4.86 (m, 1H), 4.37 (t, J = 7.8 Hz, |
| | | carboxamide | | 1H), 4.32-4.25 (m, 1H), 3.87- |
| | | | | 3.79 (m, 1H), 3.75-3.62 (m, 3H), |
| | | | | 3.61-3.48 (m, 3H), 3.47-3.39 (m, |
| | | | | 1H), 3.30-3.28 (m, 1H), 2.92- |
| | | | | 2.81 (m, 2H), 2.46 (s, 3H), 2.40- |
| | | | | 2.34 (m, 1H), 2.28-2.12 (m, 2H), |
| | | | | 2.07-1.97 (m, 1H), 1.84-1.66 (m, |
| | | | | 3H), 1.38 (d, J = 7.0 Hz, 3H), 1.31- |
| | | | | 1.18 (m, 2H), 0.95 (d, J = 6.6 Hz, |
| | | | | 3H), 0.81 (d, J = 6.6 Hz, 3H). |
| 260 | B | (2S,4R)-4-hydroxy-1-((R)-2- | 805.40 | 1H NMR (400 MHz, Methanol-d4) δ |
| | | (3-(2-((S)-2-(3-(2- | | 8.87 (s, 1H), 8.48 (s, 1H), 7.75 (d, |
| | | hydroxyphenyl)-7H- | | J = 7.7 Hz, 1H), 7.48-7.29 (m, 5H), |
| | | pyrrolo[2,3-c]pyridazin-6- | | 7.10-6.97 (m, 2H), 6.84 (s, 1H), |
| | | yl)pyrrolidin-1- | | 5.86 (s, 1H), 5.03 (q, J = 6.9 Hz, |
| | | yl)ethoxy)isoxazol-5-yl)-3- | | 1H), 4.48 (t, J = 8.3 Hz, 1H), 4.44- |
| | | methylbutanoyl)-N-((S)-1-(4- | | 4.39 (m, 1H), 4.34-4.22 (m, 2H), |
| | | (4-methylthiazol-5- | | 4.18-4.13 (m, 1H), 3.79 (dd, J = |
| | | yl)phenyl)ethyl)pyrrolidine-2- | | 10.9, 4.1 Hz, 1H), 3.66-3.49 (m, |
| | | carboxamide | | 3H), 3.14-3.00 (m, 2H), 2.78- |
| | | | | 2.73 (m, 1H), 2.46 (d, 4H), 2.30- |
| | | | | 2.12 (m, 2H), 2.10-1.89 (m, 4H), |
| | | | | 1.52 (t, J = 7.1 Hz, 3H), 0.98 (d, J = |
| | | | | 6.6, 1.6 Hz, 3H), 0.75 (d, J = 12.2, |
| | | | | 6.7 Hz, 3H). |
| 275 | B | (2S,4R)-4-hydroxy-1-((R)-2- | 805.30 | 1H NMR (300 MHz, DMSO-d6) δ |
| | | (3-(2-(3-(3-(2- | | 14.15 (s, 1H), 12.55 (br s, 1H), 8.98 |
| | | hydroxyphenyl)-7H- | | (s, 1H), 8.55 (d, J = 4.1 Hz, 1H), |
| | | pyrrolo[2,3-c]pyridazin-6-yl)- | | 8.41 (d, J = 7.7 Hz, 1H), 8.08-7.98 |
| | | 3-methylazetidin-1- | | (m, 1H), 7.49-7.40 (m, 2H), 7.36 |
| | | yl)ethoxy)isoxazol-5-yl)-3- | | (d, J = 8.2 Hz, 2H), 7.33-7.24 (m, |
| | | methylbutanoyl)-N-((S)-1-(4- | | 1H), 6.97 (t, J = 7.8 Hz, 2H), 6.85- |
| | | (4-methylthiazol-5- | | 6.51 (m, 1H), 6.09 (s, 1H), 5.10 (d, |
| | | yl)phenyl)ethyl)pyrrolidine-2- | | J = 3.6 Hz, 1H), 4.91 (t, J = 6.9 Hz, |
| | | carboxamide | | 1H), 4.37 (t, J = 7.9 Hz, 1H), 4.33- |
| | | | | 4.25 (m, 1H), 4.19-4.08 (m, 2H), |
| | | | | 3.76-3.50 (m, 4H), 3.49-3.41 (m, |
| | | | | 1H), 3.41-3.38 (m, 1H), 2.86 (t, |
| | | | | J = 2.7 Hz, 2H), 2.48-2.45 (m, 3H), |
| | | | | 2.20-1.97 (m, 2H), 1.87-1.74 |
| | | | | (m, 1H), 1.70 (s, 3H), 1.41 (dd, J = |
| | | | | 21.1, 7.0 Hz, 3H), 1.01-0.92 (m, |
| | | | | 3H), 0.88-0.69 (m, 3H). |
| 276 | B | (2S,4R)-4-hydroxy-1-((R)-2- | 833.50 | 1H NMR (400 MHz, DMSO-d6) δ |
| | | (3-(2-(4-(3-(2- | | 13.94 (s, 1H), 12.57 (d, 1H), 9.51 |
| | | hydroxyphenyl)-7H- | | (s, 1H), 8.99 (s, 1H), 8.57 (s, 1H), |
| | | pyrrolo[2,3-c]pyridazin-6-yl)- | | 8.39 (d, J = 7.8 Hz, 1H), 8.03 (s, |
| | | 4-methylpiperidin-1- | | 1H), 7.49-7.41 (m, 2H), 7.40- |
| | | yl)ethoxy)isoxazol-5-yl)-3- | | 7.34 (m, 2H), 7.30 (t, J = 7.8 Hz, |
| | | methylbutanoyl)-N-((S)-1-(4- | | 1H), 6.97 (t, J = 7.8 Hz, 2H), 6.50 |
| | | (4-methylthiazol-5- | | (d, J = 16.0 Hz, 1H), 5.11 (s, 1H), |
| | | yl)phenyl)ethyl)pyrrolidine-2- | | 4.91 (t, J = 7.2 Hz, 1H), 4.58 (s, |
| | | carboxamide | | 1H), 4.48 (s, 1H), 4.32 (d, J = 21.8 |
| | | | | Hz, 2H), 3.75-3.64 (m, 3H), 3.62- |
| | | | | 3.53 (m, 2H), 3.50-3.39 (m, 3H), |
| | | | | 3.02-2.94 (m, 1H), 2.46 (s, 3H), |
| | | | | 2.28-2.19 (m, 3H), 2.08-1.94 (m, |
| | | | | 3H), 1.83-1.76 (m, 1H), 1.53- |
| | | | | 1.46 (m, 2H), 1.38 (d, J = 7.3 Hz, |
| | | | | 4H), 0.96 (d, J = 6.8 Hz, 3H), 0.79 |
| | | | | (d, J = 6.8 Hz, 3H). |
| 277 | B | (2S,4R)-4-hydroxy-1-((R)-2- | 835.25 | 1H NMR (300 MHz, DMSO-d6) δ |
| | | (3-(2-(4-hydroxy-4-(3-(2- | | 14.14 (s, 1H), 12.56 (s, 1H), 8.99 (s, |
| | | hydroxyphenyl)-7H- | | 1H), 8.61 (s, 1H), 8.42 (d, J = 7.6 |
| | | pyrrolo[2,3-c]pyridazin-6- | | Hz, 1H), 8.08-7.92 (m, 1H), 7.52- |
| | | yl)piperidin-1- | | 7.41 (m, 2H), 7.41-7.34 (m, 2H), |
| | | yl)ethoxy)isoxazol-5-yl)-3- | | 7.33-7.24 (m, 1H), 7.01-6.87 (m, |
| | | methylbutanoyl)-N-((S)-1-(4- | | 2H), 6.55 (d, J = 8.4 Hz, 1H), 6.16 |
| | | (4-methylthiazol-5- | | (s, 1H), 5.12 (d, J = 3.6 Hz, 1H), |
| | | yl)phenyl)ethyl)pyrrolidine-2- | | 5.00-4.65 (m, 1H), 4.64-4.03 (m, |
| | | carboxamide | | 4H), 3.78-3.66 (m, 2H), 3.64- |
| | | | | 3.43 (m, 2H), 2.95-2.75 (m, 1H), |
| | | | | 2.54-2.52 (m, 3H), 2.47-2.45 (m, |
| | | | | 4H), 2.28-2.10 (m, 3H), 2.09- |
| | | | | 1.72 (m, 4H), 1.54-1.33 (m, 3H), |
| | | | | 0.97 (d, J = 6.4 Hz, 3H), 0.83 (dd, |
| | | | | J = 10.4, 6.6 Hz, 3H). |
| 278 | B | (2S,4R)-4-hydroxy-1-((R)-2- | 817.60 | 1H NMR (400 MHz, DMSO-d6) δ |
| | | (3-(2-((S)-3-(3-(2- | | 14.29 (s, 1H), 12.42 (s, 1H), 8.98 (s, |
| | | hydroxyphenyl)-7H- | | 1H), 8.52 (s, 1H), 8.40 (d, J = 7.6 |
| | | pyrrolo[2,3-c]pyridazin-6- | | Hz, 1H), 8.06-7.99 (m, 1H), 7.48- |
| | | yl)piperidin-1- | | 7.40 (m, 2H), 7.39-7.32 (m, 2H), |
| | | yl)ethoxy)isoxazol-5-yl)-3- | | 7.32-7.23 (m, 1H), 6.99-6.90 (m, |
| | | methylbutanoyl)-N-((S)-1-(4- | | 2H), 6.46 (s, 1H), 6.13 (s, 1H), 5.11 |
| | | (4-methylthiazol-5- | | (s, 1H), 4.91 (p, J = 7.1 Hz, 1H), |
| | | yl)phenyl)ethyl)pyrrolidine-2- | | 4.37 (t, J = 7.9 Hz, 1H), 4.30 (t, J = |
| | | carboxamide | | 5.4 Hz, 3H), 3.74-3.62 (m, 2H), |
| | | | | 3.53-3.42 (m, 1H), 3.21 (d, J = |
| | | | | 11.1 Hz, 1H), 3.09 (s, 1H), 2.89 (d, |
| | | | | J = 9.7 Hz, 1H), 2.77 (t, J = 5.7 Hz, |
| | | | | 2H), 2.45 (d, J = 3.2 Hz, 3H), 2.30- |
| | | | | 2.15 (m, 3H), 2.10-1.98 (m, 2H), |
| | | | | 1.84-1.71 (m, 2H), 1.61 (d, J = 8.5 |
| | | | | Hz, 2H), 1.41 (d, J = 7.0 Hz, 3H), |
| | | | | 0.96 (d, J = 6.2 Hz, 3H), 0.81 (d, J = |
| | | | | 6.6 Hz, 3H). |
| 279 | B | (2S,4R)-4-hydroxy-1-((R)-2- | 819.60 | 1H NMR (400 MHz, DMSO-d6) δ |
| | | (3-(2-((R)-3-(3-(2- | | 14.25 (s, 1H), 12.41 (s, 1H), 8.98 (s, |
| | | hydroxyphenyl)-7H- | | 1H), 8.53 (d, J = 2.6 Hz, 1H), 8.41 |
| | | pyrrolo[2,3-c]pyridazin-6- | | (d, J = 7.7 Hz, 1H), 8.02 (dt, J = 8.3, |
| | | yl)piperidin-1- | | 2.4 Hz, 1H), 7.48-7.40 (m, 2H), |
| | | yl)ethoxy)isoxazol-5-yl)-3- | | 7.40-7.33 (m, 2H), 7.28 (td, J = |
| | | methylbutanoyl)-N-((S)-1-(4- | | 8.4, 7.8, 1.7 Hz, 1H), 6.99-6.89 |
| | | (4-methylthiazol-5- | | (m, 2H), 6.47 (d, J = 2.4 Hz, 1H), |
| | | yl)phenyl)ethyl)pyrrolidine-2- | | 6.14 (s, 1H), 5.12 (s, 1H), 4.91 (p, |
| | | carboxamide | | J = 7.1 Hz, 1H), 4.37 (t, J = 7.9 Hz, |
| | | | | 1H), 4.30 (t, J = 5.6 Hz, 3H), 3.74- |
| | | | | 3.61 (m, 2H), 3.53-3.42 (m, 1H), |
| | | | | 3.19 (d, J = 11.1 Hz, 1H), 3.10 (s, |
| | | | | 1H), 2.89 (d, J = 11.0 Hz, 1H), 2.77 |
| | | | | (t, J = 5.6 Hz, 2H), 2.45 (d, J = 2.7 |
| | | | | Hz, 3H), 2.31-2.14 (m, 3H), 2.08- |
| | | | | 1.98 (m, 2H), 1.80-1.75 (m, 2H), |
| | | | | 1.62 (d, J = 7.8 Hz, 2H), 1.41 (d, J = |
| | | | | 7.0 Hz, 3H), 0.95 (d, J = 6.4 Hz, |
| | | | | 3H), 0.80 (d, J = 6.7 Hz, 3H). |
| 280 | B | (2S,4R)-4-hydroxy-1-((R)-2- | 803.60 | 1H NMR (400 MHz, DMSO-d6) δ |
| | | (3-(2-((S)-3-(3-(2- | | 14.21 (s, 1H), 12.43 (s, 1H), 8.98 (s, |
| | | hydroxyphenyl)-7H- | | 1H), 8.52 (d, J = 3.5 Hz, 1H), 8.41 |
| | | pyrrolo[2,3-c]pyridazin-6- | | (d, J = 7.6 Hz, 1H), 8.02 (dd, J = |
| | | yl)pyrrolidin-1- | | 7.9, 1.7 Hz, 1H), 7.48-7.40 (m, |
| | | yl)ethoxy)isoxazol-5-yl)-3- | | 2H), 7.36 (d, J = 8.3 Hz, 2H), 7.29 |
| | | methylbutanoyl)-N-((S)-1-(4- | | (td, J = 8.3, 7.6, 1.6 Hz, 1H), 6.99- |
| | | (4-methylthiazol-5- | | 6.89 (m, 2H), 6.49 (s, 1H), 6.12 (s, |
| | | yl)phenyl)ethyl)pyrrolidine-2- | | 1H), 5.10 (d, J = 3.6 Hz, 1H), 4.91 |
| | | carboxamide | | (p, J = 6.9 Hz, 1H), 4.37 (t, J = 7.9 |
| | | | | Hz, 1H), 4.29 (t, J = 5.6 Hz, 3H), |
| | | | | 3.74-3.55 (m, 3H), 3.45 (d, J = |
| | | | | 10.5 Hz, 1H), 3.15 (t, J = 8.4 Hz, |
| | | | | 1H), 2.94-2.79 (m, 3H), 2.73 (dt, |
| | | | | J = 9.2, 5.9 Hz, 2H), 2.45 (d, J = 3.5 |
| | | | | Hz, 3H), 2.33-2.13 (m, 2H), 2.01 |
| | | | | (d, J = 7.6 Hz, 2H), 1.78 (ddd, J = |
| | | | | 12.7, 7.9, 4.7 Hz, 1H), 1.41 (d, J = |
| | | | | 7.0 Hz, 3H), 0.96 (d, J = 6.2 Hz, |
| | | | | 3H), 0.81 (d, J = 6.7 Hz, 3H). |
| 281 | B | (1R,4S)-4-hydroxy-2-((R)-2- | 803.60 | 1H NMR (400 MHz, DMSO-d6) δ |
| | | (3-(2-((R)-3-(3-(2- | | 14.21 (s, 1H), 12.45 (s, 1H), 8.98 (s, |
| | | hydroxyphenyl)-7H- | | 1H), 8.52 (d, J = 3.4 Hz, 1H), 8.41 |
| | | pyrrolo[2,3-c]pyridazin-6- | | (d, J = 7.7 Hz, 1H), 8.02 (dd, J = |
| | | yl)pyrrolidin-1- | | 8.0, 1.7 Hz, 1H), 7.48-7.40 (m, |
| | | yl)ethoxy)isoxazol-5-yl)-3- | | 2H), 7.36 (d, J = 8.1 Hz, 2H), 7.33- |
| | | methylbutanoyl)-N-((S)-1-(4- | | 7.24 (m, 1H), 6.99-6.89 (m, 2H), |
| | | (4-methylthiazol-5- | | 6.49 (s, 1H), 6.12 (s, 1H), 5.10 (d, |
| | | yl)phenyl)ethyl)cyclopentane- | | J = 3.6 Hz, 1H), 4.91 (p, J = 7.6 Hz, |
| | | 1-carboxamide | | 1H), 4.37 (t, J = 7.8 Hz, 1H), 4.29 (t, |
| | | | | J = 5.5 Hz, 3H), 3.74-3.54 (m, |
| | | | | 3H), 3.45 (d, J = 10.6 Hz, 1H), 3.14 |
| | | | | (t, J = 8.4 Hz, 1H), 2.87 (ddd, J = |
| | | | | 23.1, 13.3, 6.9 Hz, 3H), 2.73 (s, |
| | | | | 2H), 2.45 (d, J = 3.0 Hz, 3H), 2.32- |
| | | | | 2.18 (m, 2H), 2.02 (s, 2H), 1.78 |
| | | | | (ddd, J = 12.8, 8.1, 4.8 Hz, 1H), |
| | | | | 1.41 (d, J = 7.0 Hz, 3H), 0.96 (d, J = |
| | | | | 6.1 Hz, 3H), 0.81 (d, J = 6.7 Hz, |
| | | | | 3H). |
| 285 | B | (2S,4R)-1-((R)-2-(3-(2-(4- | 837.25 | 1H NMR (400 MHz, DMSO-d6) δ |
| | | fluoro-4-(3-(2- | | 13.83 (s, 1H), 12.82 (s, 1H), 8.99 (s, |
| | | hydroxyphenyl)-7H- | | 1H), 8.65 (d, J = 3.8 Hz, 1H), 8.43 |
| | | pyrrolo[2,3-c]pyridazin-6- | | (d, J = 7.7 Hz, 1H), 8.03 (dd, J = |
| | | yl)piperidin-1- | | 8.1, 1.7 Hz, 1H), 7.49-7.41 (m, |
| | | yl)ethoxy)isoxazol-5-yl)-3- | | 2H), 7.41-7.34 (m, 2H), 7.30 (td, |
| | | methylbutanoyl)-4-hydroxy- | | J = 7.7, 7.2, 1.6 Hz, 1H), 7.01-6.92 |
| | | N-((S)-1-(4-(4-methylthiazol- | | (m, 2H), 6.69 (d, J = 3.4 Hz, 1H), |
| | | 5-yl)phenyl)ethyl)pyrrolidine- | | 6.13 (s, 1H), 5.12 (d, J = 3.6 Hz, |
| | | 2-carboxamide | | 1H), 4.90 (q, J = 7.3 Hz, 1H), 4.41- |
| | | | | 4.26 (m, 4H), 3.74-3.62 (m, 2H), |
| | | | | 3.53-3.42 (m, 1H), 2.91 (d, J = |
| | | | | 11.2 Hz, 2H), 2.79 (t, J = 5.5 Hz, |
| | | | | 2H), 2.45 (d, J = 1.5 Hz, 4H), 2.35- |
| | | | | 2.18 (m, 5H), 2.03 (dd, J = 11.7, 8.1 |
| | | | | Hz, 1H), 1.83-1.72 (m, 1H), 1.38 |
| | | | | (d, J = 7.0 Hz, 3H), 0.96 (d, J = 6.4 |
| | | | | Hz, 3H), 0.80 (d, J = 6.7 Hz, 3H). |
| 286 | B | (2S,4R)-4-hydroxy-1-((2R)- | 819.60 | 1H NMR (400 MHz, DMSO-d6) δ |
| | | 2-(3-(2-(3-(3-(2- | | 14.20 (s, 1H), 12.47 (s, 1H), 8.98 (s, |
| | | hydroxyphenyl)-7H- | | 1H), 8.53 (d, J = 3.6 Hz, 1H), 8.40 |
| | | pyrrolo[2,3-c]pyridazin-6- | | (d, J = 7.6 Hz, 1H), 8.02 (dt, J = 9.4, |
| | | yl)piperidin-1- | | 2.9 Hz, 1H), 7.48-7.40 (m, 2H), |
| | | yl)ethoxy)isoxazol-5-yl)-3- | | 7.40-7.33 (m, 2H), 7.28 (ddd, J = |
| | | methylbutanoyl)-N-((S)-1-(4- | | 8.5, 7.2, 1.6 Hz, 1H), 6.95 (td, J = |
| | | (4-methylthiazol-5- | | 7.8, 7.2, 1.4 Hz, 2H), 6.47 (d, J = |
| | | yl)phenyl)ethyl)pyrrolidine-2- | | 2.1 Hz, 1H), 6.14 (d, J = 1.3 Hz, |
| | | carboxamide | | 1H), 5.14-4.86 (m, 2H), 4.37 (t, |
| | | | | J = 7.9 Hz, 1H), 4.30 (t, J = 5.5 Hz, |
| | | | | 3H), 3.70 (dd, J = 10.5, 4.4 Hz, 1H), |
| | | | | 3.65 (d, J = 9.8 Hz, 1H), 3.53-3.42 |
| | | | | (m, 1H), 3.21 (s, 1H), 3.10 (s, 1H), |
| | | | | 2.89 (d, J = 10.9 Hz, 1H), 2.77 (t, |
| | | | | J = 5.7 Hz, 2H), 2.45 (d, J = 2.9 Hz, |
| | | | | 3H), 2.31-2.12 (m, 3H), 2.08- |
| | | | | 1.98 (m, 2H), 1.78 (ddd, J = 19.4, |
| | | | | 9.6, 5.6 Hz, 2H), 1.61 (s, 2H), 1.47- |
| | | | | 1.33 (m, 3H), 0.96 (d, J = 6.4 Hz, |
| | | | | 3H), 0.81 (d, J = 6.7 Hz, 3H). |
| 287 | B | (2S,4R)-1-((R)-2-(3-(2-(3-(3- | 843.40 | 1H NMR (400 MHz, DMSO-d6) δ |
| | | (3-chloro-5-fluoro-2- | | 8.98 (d, J = 2.2 Hz, 1H), 8.71 (d, J = |
| | | hydroxyphenyl)-7H- | | 6.0 Hz, 1H), 8.42 (d, J = 7.7 Hz, |
| | | pyrrolo[2,3-c]pyridazin-6- | | 1H), 8.01 (td, J = 9.6, 9.0, 3.1 Hz, |
| | | yl)azetidin-1- | | 1H), 7.51-7.40 (m, 3H), 7.40- |
| | | yl)ethoxy)isoxazol-5-yl)-3- | | 7.33 (m, 2H), 6.62 (s, 1H), 6.09 (s, |
| | | methylbutanoyl)-4-hydroxy- | | 1H), 5.13 (s, 1H), 4.92 (q, J = 6.8 |
| | | N-((S)-1-(4-(4-methylthiazol- | | Hz, 1H), 4.37 (t, J = 7.9 Hz, 1H), |
| | | 5-yl)phenyl)ethyl)pyrrolidine- | | 4.29 (s, 1H), 4.16 (t, J = 5.3 Hz, |
| | | 2-carboxamide | | 2H), 3.91 (q, J = 7.4 Hz, 1H), 3.78- |
| | | | | 3.62 (m, 4H), 3.60-3.38 (m, 4H), |
| | | | | 2.86 (t, J = 5.4 Hz, 2H), 2.45 (d, J = |
| | | | | 4.5 Hz, 3H), 2.26-2.21 (m, 1H), |
| | | | | 2.03 (ddd, J = 11.5, 7.9, 2.9 Hz, |
| | | | | 1H), 1.78 (ddd, J = 12.8, 8.1, 4.8 |
| | | | | Hz, 1H), 1.41 (d, J = 7.0 Hz, 3H), |
| | | | | 0.97 (d, J = 6.4 Hz, 3H), 0.80 (d, J = |
| | | | | 6.7 Hz, 3H). |
| 288 | B | (2S,4R)-1-((R)-2-(3-(2-(3-(3- | 809.20 | 1H NMR (400 MHz, DMSO-d6) δ |
| | | (5-fluoro-2-hydroxyphenyl)- | | 13.74 (s, 1H), 12.77 (s, 1H), 8.99 (s, |
| | | 7H-pyrrolo[2,3-c]pyridazin-6- | | 1H), 8.67 (s, 1H), 8.40 (d, J = 7.6 |
| | | yl)azetidin-1- | | Hz, 1H), 8.13 (s, 1H), 7.93 (dd, J = |
| | | yl)ethoxy)isoxazol-5-yl)-3- | | 10.6, 3.1 Hz, 1H), 7.49-7.40 (m, |
| | | methylbutanoyl)-4-hydroxy- | | 2H), 7.40-7.33 (m, 2H), 7.15 (td, |
| | | N-((S)-1-(4-(4-methylthiazol- | | J = 8.5, 8.1, 3.0 Hz, 1H), 6.98 (dd, J = |
| | | 5-yl)phenyl)ethyl)pyrrolidine- | | 9.0, 5.1 Hz, 1H), 6.52 (s, 1H), 6.14 |
| | | 2-carboxamide | | (s, 1H), 5.12 (d, J = 3.4 Hz, 1H), |
| | | | | 4.92 (q, J = 7.1 Hz, 1H), 4.73-4.07 |
| | | | | (m, 9H), 3.69 (t, J = 8.4 Hz, 2H), |
| | | | | 3.61-3.38 (m, 2H), 2.45 (s, 3H), |
| | | | | 2.31-2.14 (m, 1H), 2.10-1.98 (m, |
| | | | | 1H), 1.78 (ddd, J = 12.4, 7.9, 4.6 |
| | | | | Hz, 1H), 1.42 (d, J = 7.0 Hz, 3H), |
| | | | | 0.97 (d, J = 6.4 Hz, 3H), 0.83 (d, J = |
| | | | | 6.6 Hz, 3H). |
| 289 | B | (2S,4R)-4-hydroxy-1-((R)-2- | 833.30 | 1H NMR (300 MHz, DMSO-d6) δ |
| | | (3-(2-(3-(2-(3-(2- | | 14.12 (br s, 1H), 12.45 (s, 1H), 8.99 |
| | | hydroxyphenyl)-7H- | | (s, 1H), 8.53 (d, J = 3.7 Hz, 1H), |
| | | pyrrolo[2,3-c]pyridazin-6- | | 8.41 (d, J = 7.6 Hz, 1H), 8.05-7.98 |
| | | yl)propan-2-yl)azetidin-1- | | (m, 1H), 7.49-7.41 (m, 2H), 7.37 |
| | | yl)ethoxy)isoxazol-5-yl)-3- | | (d, J = 8.4 Hz, 2H), 7.55-7.24 (m, |
| | | methylbutanoyl)-N-((S)-1-(4- | | 1H), 6.96 (t, J = 7.5 Hz, 2H), 6.39 |
| | | (4-methylthiazol-5- | | (s, 1H), 6.06 (s, 1H), 5.10 (s, 1H), |
| | | yl)phenyl)ethyl)pyrrolidine-2- | | 4.97-4.84 (m, 1H), 4.37 (t, J = 7.9 |
| | | carboxamide | | Hz, 1H), 4.28 (s, 1H), 4.07 (t, J = |
| | | | | 5.4 Hz, 2H), 3.77-3.59 (m, 2H), |
| | | | | 3.61-3.49 (m, 2H), 3.04 (t, J = 7.0 |
| | | | | Hz, 1H), 2.95 (t, J = 7.0 Hz, 1H), |
| | | | | 2.71-2.65 (m, 2H), 2.46 (s, 3H), |
| | | | | 2.31-2.17 (m, 1H), 2.10-1.95 (m, |
| | | | | 1H), 1.85-1.71 (m, 1H), 1.49-1.31 |
| | | | | (m, 9H), 1.01-0.91 (m, 3H), 0.85- |
| | | | | 0.74 (m, 3H). |
| 290 | B | (2S,4R)-4-hydroxy-1-((R)-2- | 859.30 | 1H NMR (300 MHz, DMSO-d6) δ |
| | | (3-(2-(2-(3-(2- | | 14.29 (br s, 1H), 12.52 (s, 1H), 8.99 |
| | | hydroxyphenyl)-7H- | | (s, 1H), 8.52 (s, 1H), 8.42 (d, J = |
| | | pyrrolo[2,3-c]pyridazin-6-yl)- | | 7.7 Hz, 1H), 8.17 (s, 1H, FA), 8.09- |
| | | 7-azaspiro[3.5]nonan-7- | | 7.93 (m, 1H), 7.59-7.12 (m, 5H), |
| | | yl)ethoxy)isoxazol-5-yl)-3- | | 6.95 (t, J = 7.8 Hz, 2H), 6.61-6.45 |
| | | methylbutanoyl)-N-((S)-1-(4- | | (m, 1H), 6.18-5.91 (m, 1H), 5.11 |
| | | (4-methylthiazol-5- | | (s, 1H), 4.93 (q, J = 7.2 Hz, 1H), |
| | | yl)phenyl)ethyl)pyrrolidine-2- | | 4.38 (t, J = 7.8 Hz, 1H), 4.54-4.18 |
| | | carboxamide | | (m, 3H), 3.86-3.59 (m, 4H), 3.58- |
| | | | | 3.38 (m, 3H), 2.72-2.57 (m, 2H), |
| | | | | 2.46 (s, 3H), 2.42-2.30 (m, 2H), |
| | | | | 2.24 (s, 1H), 2.15-2.98 (m, 3H), |
| | | | | 1.87-1.68 (m, 3H), 1.66-1.55 (m, |
| | | | | 2H), 1.42 (d, J = 7.0 Hz, 3H), 0.97 |
| | | | | (d, J = 6.3 Hz, 3H), 0.81 (d, J = 6.7 |
| | | | | Hz, 3H). |
| 294 | B | (2S,4R)-4-hydroxy-1-((R)-2- | 803.60 | 1H NMR (300 MHz, DMSO-d6) δ |
| | | (3-(2-(3-((3-(2- | | 14.14 (d, J = 11.5 Hz, 1H), 12.51 (s, |
| | | hydroxyphenyl)-7H- | | 1H), 8.98 (s, 1H), 8.53 (d, J = 3.6 |
| | | pyrrolo[2,3-c]pyridazin-6- | | Hz, 1H), 8.40 (d, J = 7.7 Hz, 1H), |
| | | yl)methyl)azetidin-1- | | 8.14 (s, 1H), 8.06-7.97 (m, 1H), |
| | | yl)ethoxy)isoxazol-5-yl)-3- | | 7.48-7.34 (m, 4H), 7.29 (m, J = |
| | | methylbutanoyl)-N-((S)-1-(4- | | 7.7, 7.1, 1.5 Hz, 1H), 6.95 (m, J = |
| | | (4-methylthiazol-5- | | 8.2, 6.8 Hz, 2H), 6.52 (s, 1H), 6.41 |
| | | yl)phenyl)ethyl)pyrrolidine-2- | | (s, 1H), 6.10 (s, 1H), 5.10 (d, J = |
| | | carboxamide | | 3.6 Hz, 1H), 4.91 (t, J = 7.2 Hz, 1H), |
| | | | | 4.36 (t, J = 7.9 Hz, 1H), 4.28 (s, |
| | | | | 1H), 4.19 (s, 2H), 3.80-3.40 (m, |
| | | | | 5H), 3.16 (d, J = 7.7 Hz, 2H), 3.02 |
| | | | | (s, 3H), 2.45 (s, 3H), 2.31-1.86 |
| | | | | (m, 3H), 1.85-1.71 (m, 1H), 1.41 |
| | | | | (d, 7.0 Hz, 3H), 0.96 (d, J = 6.7Hz, |
| | | | | 3H), 0.81 (d, J = 6.7 Hz, 3H). |
| 308 | B | (2S,4R)-4-hydroxy-1-((R)-2- | 817.45 | 1H NMR (300 MHz, DMSO-d6) δ |
| | | (3-(2-((1R,5S,6R)-6-(3-(2- | | 14.33 (s, 1H), 12.41 (d, J = 1.9 Hz, |
| | | hydroxyphenyl)-7H- | | 1H), 8.98 (d, J = 4.8 Hz, 1H), 8.43 |
| | | pyrrolo[2,3-c]pyridazin-6-yl)- | | (d, J = 8.7 Hz, 2H), 8.07-7.96 (m, |
| | | 3-azabicyclo[3.1.0]hexan-3- | | 1H), 7.45 (d, J = 8.3 Hz, 2H), 7.37 |
| | | yl)ethoxy)isoxazol-5-yl)-3- | | (d, J = 8.3 Hz, 2H), 7.33-7.22 (m, |
| | | methylbutanoyl)-N-((S)-1-(4- | | 1H), 6.99-6.89 (m, 2H), 6.34- |
| | | (4-methylthiazol-5- | | 6.28 (m, 1H), 6.13 (s, 1H), 5.12 (dd, |
| | | yl)phenyl)ethyl)pyrrolidine-2- | | J = 3.6, 6.2 Hz, 1H), 4.92 (t, J = 7.2 |
| | | carboxamide | | Hz, 1H), 4.38 (t, J = 7.9 Hz, 1H), |
| | | | | 4.54-4.20 (m, 3H), 3.78-3.54 (m, |
| | | | | 2H), 3.53-3.42 (m, 1H), 3.23 (d, |
| | | | | J = 9.1 Hz, 2H), 2.92-2.82 (m, 2H), |
| | | | | 2.61-2.53 (m, 2H), 2.48-2.42 (m, |
| | | | | 4H), 2.35-2.17 (m, 1H), 2.15- |
| | | | | 2.05 (m, 2H), 2.04-1.96 (m, 1H), |
| | | | | 1.95-1.67 (m, 1H), 1.43 (dd, J = |
| | | | | 24.8, 7.0 Hz, 3H), 1.08-0.95 (m, |
| | | | | 3H), 0.91-0.78 (m, 3H). |
| 216 | B | (2S,4R)-4-hydroxy-1-((R)-2- | 831.50 | 1H NMR (300 MHz, DMSO-d6) δ |
| | | (3-(3-((1R,5S,6R)-6-(3-(2- | | 14.34 (s, 1H), 12.42 (s, 1H), 8.96 |
| | | hydroxyphenyl)-7H- | | (d, J = 13.5 Hz, 1H), 8.48-8.38 (m, |
| | | pyrrolo[2,3-c]pyridazin-6-yl)- | | 2H), 8.01 (dd, J = 7.6, 5.8 Hz, 1H), |
| | | 3-azabicyclo[3.1.0]hexan-3- | | 7.49-7.33 (m, 4H), 7.32-7.22 (m, |
| | | yl)propoxy)isoxazol-5-yl)-3- | | 1H), 7.00-6.89 (m, 2H), 6.30 (d, |
| | | methylbutanoyl)-N-((S)-1-(4- | | J = 11.0 Hz, 1H), 5.78 (s, 1H), 5.13 |
| | | (4-methylthiazol-5- | | (s, 1H), 4.92 (t, J = 7.2 Hz, 1H), |
| | | yl)phenyl)ethyl)pyrrolidine-2- | | 4.41 (t, J = 7.9 Hz, 1H), 4.30 (s, |
| | | carboxamide | | 1H), 3.84 (t, J = 6.6 Hz, 2H), 3.69 |
| | | | | (dd, J = 10.5, 4.4 Hz, 1H), 3.57 (d, |
| | | | | J = 9.7 Hz, 1H), 3.48 (t, J = 9.6 Hz, |
| | | | | 1H), 3.18 (t, J = 8.8 Hz, 2H), 2.46 |
| | | | | (s, 3H), 2.39 (d, J = 7.4 Hz, 3H), |
| | | | | 2.23 (d, J = 7.2 Hz, 1H), 2.09 (s, |
| | | | | 3H), 1.87-1.69 (m, 3H), 1.39 (d, J = |
| | | | | 7.0 Hz, 3H), 0.97 (d, J = 6.3 Hz, |
| | | | | 3H), 0.89 (d, J = 6.6 Hz, 3H). |
| 117 | B | (2S,4R)-N-((S)-1-(2′-fluoro- | 788.30 | 1H NMR (300 MHz, Methanol-d4) δ |
| | | [1,1′-biphenyl]-4-yl)ethyl)-4- | | 8.42 (s, 1H), 7.93 (d, J = 8.2, 1.6 |
| | | hydroxy-1-((2S)-2-(3-(2-(3- | | Hz, 1H), 7.50-7.24 (m, 7H), 7.23- |
| | | (3-(2-hydroxyphenyl)-7H- | | 7.08 (m, 2H), 7.04-6.93 (m, 2H), |
| | | pyrrolo[2,3-c]pyridazin-6- | | 6.57 (s, 1H), 6.08 (s, 1H), 5.04- |
| | | yl)azetidin-1-yl)ethoxy)-4,5- | | 4.95 (m, 1H), 4.60 (t, J = 8.0 Hz, |
| | | dihydroisoxazol-5-yl)-3- | | 1H), 4.51-4.40 (m, 1H), 4.29 (t, |
| | | methylbutanoyl)pyrrolidine- | | J = 5.2 Hz, 2H), 4.05-3.94 (m, 1H), |
| | | 2-carboxamide | | 3.95-3.85 (m, 2H), 3.84-3.74 (m, |
| | | | | 1H), 3.74-3.62 (m, 2H), 3.60- |
| | | | | 3.50 (m, 2H), 3.01-2.94 (m, 2H), |
| | | | | 2.49-2.32 (m, 1H), 2.31-2.13 (m, |
| | | | | 1H), 2.08-1.93 (m, 1H), 1.49 (d, |
| | | | | J = 7.0 Hz, 3H), 1.08 (d, J = 6.6 Hz, |
| | | | | 3H), 0.94 (d, J = 6.7 Hz, 3H). |
| 162 | B | (2S,4R)-4-hydroxy-1-((S)-2- | 816.25 | 1H NMR (400 MHz, DMSO-d6) δ |
| | | (3-(3-((3-(3-(2- | | 14.14 (s, 1H), 12.53 (s, 1H), 9.01- |
| | | hydroxyphenyl)-7H- | | 8.95 (m, 1H), 8.57-8.53 (m, 1H), |
| | | pyrrolo[2,3-c]pyridazin-6- | | 8.22 (d, J = 7.9 Hz, 1H), 8.06-7.99 |
| | | yl)azetidin-1- | | (m, 1H), 7.50-7.24 (m, 5H), 7.00- |
| | | yl)methyl)azetidin-1- | | 6.90 (m, 2H), 6.58-6.52 (m, 1H), |
| | | yl)isoxazol-5-yl)-3- | | 5.89-5.84 (m, 1H), 5.14-4.83 (m, |
| | | methylbutanoyl)-N-((S)-1-(4- | | 2H), 4.42 (t, J = 7.7 Hz, 1H), 4.27 |
| | | (4-methylthiazol-5- | | (s, 1H), 3.96-3.80 (m, 3H), 3.73- |
| | | yl)phenyl)ethyl)pyrrolidine-2- | | 3.59 (m, 3H), 3.56-3.40 (m, 4H), |
| | | carboxamide | | 3.35-3.31 (m, 2H), 2.76-2.61 (m, |
| | | | | 3H), 2.48-2.41 (m, 3H), 2.30- |
| | | | | 2.17 (m, 1H), 2.09-1.98 (m, 1H), |
| | | | | 1.85-1.74 (m, 1H), 1.40 (dd, J = |
| | | | | 40.2, 7.0 Hz, 3H), 0.98-0.70 (m, |
| | | | | 6H). |
| 202 | B | (2S,4R)-4-hydroxy-1-((S)-2- | 789.45 | 1H NMR (300 MHz, Methanol-d4) δ |
| | | (3-(3-(3-(3-(2- | | 8.85 (d, J = 28.2 Hz, 1H), 8.41 (d, J = |
| | | hydroxyphenyl)-7H- | | 29.6 Hz, 1H), 7.99-7.83 (m, 1H), |
| | | pyrrolo[2,3-c]pyridazin-6- | | 7.53-7.24 (m, 5H), 7.04-6.92 (m, |
| | | yl)azetidin-1- | | 2H), 6.56 (d, J = 22.7 Hz, 1H), 6.31 |
| | | yl)propyl)isoxazol-5-yl)-3- | | (d, J = 16.3 Hz, 1H), 5.04-4.91 (m, |
| | | methylbutanoyl)-N-((S)-1-(4- | | 1H), 4.66-4.53 (m, 1H), 4.46 (s, |
| | | (4-methylthiazol-5- | | 1H), 4.05-3.92 (m, 1H), 3.92- |
| | | yl)phenyl)ethyl)pyrrolidine-2- | | 3.79 (m, 3H), 3.78-3.69 (m, 1H), |
| | | carboxamide | | 3.68-3.59 (m, 1H), 3.52-3.45 (m, |
| | | | | 1H), 3.45-3.37 (m, 1H), 2.73 (t, J = |
| | | | | 7.3 Hz, 2H), 2.65 (t, J = 7.6 Hz, |
| | | | | 2H), 2.53-2.34 (m, 4H), 2.31- |
| | | | | 2.17 (m, 1H), 2.04 (d, J = 13.1, 8.2, |
| | | | | 4.8 Hz, 1H), 1.89-1.73 (m, 2H), |
| | | | | 1.54 (dd, J = 38.8, 7.0 Hz, 3H), 1.03 |
| | | | | (dd, J = 33.1, 6.6 Hz, 3H), 0.88 (dd, |
| | | | | J = 20.9, 6.7 Hz, 3H). |
| 207 | B | (2S,4R)-4-hydroxy-1-((S)-2- | 777.25 | 1H NMR (300 MHz, DMSO-d6) δ |
| | | (3-(2-(3-(3-(2- | | 14.17 (s, 1H), 12.54 (s, 1H), 8.98 |
| | | hydroxyphenyl)-7H- | | (d, J = 5.3 Hz, 1H), 8.58-8.43 (m, |
| | | pyrrolo[2,3-c]pyridazin-6- | | 2H), 8.03 (dd, J = 8.2, 1.7 Hz, 1H), |
| | | yl)azetidin-1- | | 7.50-7.24 (m, 5H), 6.96 (t, J = 7.9 |
| | | yl)ethoxy)isoxazol-5-yl)-3- | | Hz, 2H), 6.55 (d, J = 8.5 Hz, 1H), |
| | | methylbutanoyl)-N-(4-(4- | | 6.07 (s, 1H), 5.18-4.95 (m, 1H), |
| | | methylthiazol-5- | | 4.44 (t, J = 7.7 Hz, 1H), 4.38-4.22 |
| | | yl)benzyl)pyrrolidine-2- | | (m, 3H), 4.19-4.08 (m, 2H), 3.85 |
| | | carboxamide | | (q, J = 7.3 Hz, 1H), 3.80-3.64 (m, |
| | | | | 3H), 3.63-3.55 (m, 2H), 3.54- |
| | | | | 3.36 (m, 2H), 2.90-2.71 (m, 2H), |
| | | | | 2.44 (s, 3H), 2.34-2.13 (m, 1H), |
| | | | | 2.11-2.01 (m, 1H), 1.97-1.83 (m, |
| | | | | 1H), 1.02-0.55 (m, 6H). |
| 215 | B | (2S,4R)-4-hydroxy-1-((S)-2- | 831.50 | 1H NMR (300 MHz, DMSO-d6) δ |
| | | (3-(3-((1R,5S,6S)-6-(3-(2- | | 14.08 (s, 1H), 12.62 (s, 1H), 8.86 (s, |
| | | hydroxyphenyl)-7H- | | 1H), 8.58-8.38 (m, 2H), 8.00 (d, |
| | | pyrrolo[2,3-c]pyridazin-6-yl)- | | J = 7.7 Hz, 1H), 7.49-7.24 (m, 6H), |
| | | 3-azabicyclo[3.1.0]hexan-3- | | 6.97 (d, J = 8.0 Hz, 2H), 6.31 (s, |
| | | yl)propoxy)isoxazol-5-yl)-3- | | 1H), 5.85 (s, 1H), 5.15 (s, 1H), 4.91 |
| | | methylbutanoyl)-N-((S)-1-(4- | | (t, J = 7.3 Hz, 1H), 4.47 (s, 1H), |
| | | (4-methylthiazol-5- | | 4.30 (s, 1H), 3.87 (s, 3H), 3.65 (t, |
| | | yl)phenyl)ethyl)pyrrolidine-2- | | J = 12.3 Hz, 2H), 3.51-3.33 (m, 6H), |
| | | carboxamide | | 2.35 (s, 5H), 2.16-1.91 (m, 4H), |
| | | | | 1.86-1.65 (m, 2H), 1.38 (d, J = 7.0 |
| | | | | Hz, 3H), 0.99 (d, J = 6.6 Hz, 3H), |
| | | | | 0.92 (d, J = 6.7 Hz, 3H). |
| 218 | B | (2S,4R)-4-hydroxy-1-((S)-2- | 792.45 | 1H NMR (300 MHz, DMSO-d6) δ |
| | | (3-(2-(3-(3-(2- | | 14.23 (s, 1H), 12.62 (s, 1H), 9.07 |
| | | hydroxyphenyl)-7H- | | (d, J = 8.2 Hz, 1H), 8.72-8.40 (m, |
| | | pyrrolo[2,3-c]pyridazin-6- | | 3H), 8.10 (d, J = 7.8 Hz, 1H), 7.98- |
| | | yl)azetidin-1- | | 7.67 (m, 2H), 7.54-7.20 (m, 2H), |
| | | yl)ethoxy)isoxazol-5-yl)-3- | | 7.04 (d, J = 8.2 Hz, 2H), 6.62 (d, J = |
| | | methylbutanoyl)-N-((S)-1-(6- | | 7.9 Hz, 1H), 6.18 (d, J = 15.4 Hz, |
| | | (4-methylthiazol-5-yl)pyridin- | | 1H), 5.26-4.90 (m, 2H), 4.71- |
| | | 3-yl)ethyl)pyrrolidine-2- | | 4.42 (m, 1H), 4.33 (s, 1H), 4.28- |
| | | carboxamide | | 4.14 (m, 2H), 4.03-3.88 (m, 1H), |
| | | | | 3.88-3.71 (m, 3H), 3.68-3.60 (m, |
| | | | | 1H), 3.59-3.50 (m, 1H), 3.50- |
| | | | | 3.46 (m, 1H), 2.98-2.86 (m, 2H), |
| | | | | 2.85-2.77 (m, 2H), 2.72 (d, J = 5.6 |
| | | | | Hz, 3H), 2.18-2.04 (m, 1H), 1.91- |
| | | | | 1.77 (m, 1H), 1.56 (d, J = 7.1 Hz, |
| | | | | 1H), 1.50-1.40 (m, 3H), 1.04 (d, |
| | | | | J = 6.6 Hz, 3H), 0.90 (d, J = 6.4 Hz, |
| | | | | 3H). |
| 237 | B | (2S,4R)-4-hydroxy-1-((R)-2- | 805.50 | 1H NMR (300 MHz, DMSO-d6) δ |
| | | (3-(3-(3-(3-(2- | | 12.77 (d, J = 35.0 Hz, 1H), 10.25 (d, |
| | | hydroxyphenyl)-7H- | | J = 43.7 Hz, 1H), 8.90 (d, J = 21.5 |
| | | pyrrolo[2,3-c]pyridazin-6- | | Hz, 1H), 8.62 (s, 1H), 8.50 (d, J = |
| | | yl)azetidin-1- | | 8.0 Hz, 1H), 8.01 (d, J = 7.8 Hz, |
| | | yl)propoxy)isoxazol-5-yl)-3- | | 1H), 7.52-7.25 (m, 6H), 7.05- |
| | | methylbutanoyl)-N-((S)-1-(4- | | 6.91 (m, 2H), 6.89-6.67 (m, 1H), |
| | | (4-methylthiazol-5- | | 5.86 (s, 1H), 5.09 (d, J = 39.7 Hz, |
| | | yl)phenyl)ethyl)pyrrolidine-2- | | 1H), 4.92-4.82 (m, 1H), 4.46 (s, |
| | | carboxamide | | 3H), 4.32 (s, 4H), 3.90 (t, J = 6.5 |
| | | | | Hz, 2H), 3.67 (d, J = 9.0 Hz, 2H), |
| | | | | 3.32 (s, 2H), 2.48-2.33 (m, 3H), |
| | | | | 2.08 (s, 1H), 1.98-1.68 (m, 3H), |
| | | | | 1.50-1.26 (m, 3H), 0.98 (d, J = 6.5 |
| | | | | Hz, 3H), 0.91 (d, J = 6.6 Hz, 3H), |
| | | | | 0.83 (s, 1H). |
| 257 | B | (2S,4R)-4-hydroxy-1-((S)-2- | 804.50 | 1H NMR (300 MHz, Methanol-d4) δ |
| | | (3-((2-(3-(3-(2- | | 8.79 (s, 1H), 8.34 (s, 1H), 7.99- |
| | | hydroxyphenyl)-7H- | | 7.83 (m, 1H), 7.49-7.23 (m, 5H), |
| | | pyrrolo[2,3-c]pyridazin-6- | | 7.04-6.90 (m, 2H), 6.51 (s, 1H), |
| | | yl)azetidin-1- | | 6.13 (s, 1H), 5.15-4.92 (m, 2H), |
| | | yl)ethyl)(methyl)amino)isoxa | | 4.61 (t, J = 8.0 Hz, 1H), 4.46 (s, |
| | | zol-5-yl)-3-methylbutanoyl)- | | 1H), 3.97 (p, J = 7.2 Hz, 1H), 3.84 |
| | | N-((S)-1-(4-(4-methylthiazol- | | (t, J = 7.5 Hz, 2H), 3.77-3.57 (m, |
| | | 5-yl)phenyl)ethyl)pyrrolidine- | | 3H), 3.47 (s, 2H), 2.96 (d, J = 6.3 |
| | | 2-carboxamide | | Hz, 3H), 2.81 (d, J = 4.4 Hz, 2H), |
| | | | | 2.44 (d, J = 32.7 Hz, 3H), 2.24 (dd, |
| | | | | J = 12.0, 8.3 Hz, 1H), 2.07 (dt, J = |
| | | | | 13.0, 4.5 Hz, 1H), 1.45 (d, J = 7.0 |
| | | | | Hz, 3H), 1.31 (s, 1H), 1.07 (d, J = |
| | | | | 6.6 Hz, 3H), 0.93 (d, J = 6.8 Hz, |
| | | | | 3H). |
| 204 | B | (2S,4R)-4-hydroxy-1-((R)-2- | 789.50 | 1H NMR (300 MHz, Methanol-d4) δ |
| | | (3-(3-(3-(3-(2- | | 8.84 (d, J = 28.1 Hz, 1H), 8.38 (d, |
| | | hydroxyphenyl)-7H- | | J = 47.3 Hz, 1H), 8.03-7.80 (m, 1H), |
| | | pyrrolo[2,3-c]pyridazin-6- | | 7.44 (d, J = 1.7 Hz, 3H), 7.37-7.19 |
| | | yl)azetidin-1- | | (m, 2H), 7.06-6.88 (m, 2H), 6.54 |
| | | yl)propyl)isoxazol-5-yl)-3- | | (d, J = 38.3 Hz, 1H), 6.28 (d, J = |
| | | methylbutanoyl)-N-((S)-1-(4- | | 18.6 Hz, 1H), 5.13-5.00 (m, 1H), |
| | | (4-methylthiazol-5- | | 4.53 (t, J = 8.2 Hz, 1H), 4.46 (s, |
| | | yl)phenyl)ethyl)pyrrolidine-2- | | 1H), 4.10-3.94 (m, 1H), 3.94- |
| | | carboxamide | | 3.75 (m, 4H), 3.70-3.58 (m, 1H), |
| | | | | 3.57-3.39 (m, 2H), 2.83-2.58 (m, |
| | | | | 4H), 2.49 (s, 2H), 2.48-2.37 (m, |
| | | | | 1H), 2.36 (s, 1H), 2.29-2.11 (m, |
| | | | | 1H), 2.04-1.91 (m, 1H), 1.91- |
| | | | | 1.70 (m, 2H), 1.57 (dd, J = 19.3, 7.0 |
| | | | | Hz, 3H), 1.09 (dd, J = 6.7, 1.6 Hz, |
| | | | | 3H), 0.90 (dd, J = 9.2, 6.7 Hz, 3H). |
| 300 | B | (2S,4R)-4-hydroxy-1-[(2R)-2- | 834.0 | ¹H NMR (400 MHz, DMSO-d6) δ |
| | | (3-[2-[(2-[3-[3-(2- | | 14.12 (s, 1H), 12.56 (s, 1H), 8.99 (s, |
| | | hydroxyphenyl)-7H- | | 1H), 8.57 (d, J = 3.1 Hz, 1H), 8.42 |
| | | pyrrolo[2,3-c]pyridazin-6- | | (d, J = 7.6 Hz, 1H), 8.21 (s, 2H), |
| | | yllazetidin-1- | | 8.04 (d, J = 7.9 Hz, 1H), 7.49-7.40 |
| | | ylethyl)amino]ethoxy]-1,2- | | (m, 2H), 7.39-7.25 (m, 3H), 6.96 |
| | | oxazol-5-yl)-3- | | (d, J = 8.2 Hz, 2H), 6.57 (s, 1H), |
| | | methylbutanoyl]-N-[(1S)-1- | | 6.10 (s, 1H), 5.03 (s, 1H), 4.94- |
| | | [4-(4-methyl-1,3-thiazol-5- | | 4.86 (m, 1H), 4.37 (t, J = 7.9 Hz, |
| | | yl)phenyl]ethyl]pyrrolidine-2- | | 1H), 4.31-4.18 (m, 3H), 3.92- |
| | | carboxamide | | 3.84 (m, 1H), 3.73-3.61 (m, 4H), |
| | | | | 3.53-3.40 (m, 4H), 2.96 (d, J = 5.5 |
| | | | | Hz, 2H), 2.62 (s, 4H), 2.45 (s, 3H), |
| | | | | 2.25-1.21 (m, 1H), 2.05-1.98 (m, |
| | | | | 1H), 1.83-1.70 (m, 1H), 1.37 (d, |
| | | | | J = 7.0 Hz, 3H), 0.95 (d, J = 6.5 Hz, |
| | | | | 3H), 0.78 (d, J = 6.7 Hz, 3H) |
| 295 | B | (2S,4R)-4-hydroxy-1-[(2R)-2- | | ¹H NMR (400 MHz, DMSO-d6) δ |
| | | [3-(2-[[(4-[3-(2- | | 14.20 (s, 1H), 8.98 (s, 1H), 8.51 (d, |
| | | hydroxyphenyl)-7H- | | J = 3.0 Hz, 1H), 8.41 (d, J = 7.6 Hz, |
| | | pyrrolo[2,3-c]pyridazin-6- | | 1H), 7.99 (dd, J = 8.0, 1.7 Hz, 1H), |
| | | yl]methyl]phenyl)methyl]ami- | | 7.49-7.40 (m, 2H), 7.34 (d, J = |
| | | no]ethoxy)-1,2-oxazol-5-yl]- | | 25.0 Hz, 2H), 7.32-7.23 (m, 5H), |
| | | 3-methylbutanoyl]-N-[(1S)-1- | | 6.99-6.89 (m, 2H), 6.34 (s, 1H), |
| | | [4-(4-methyl-1,3-thiazol-5- | | 6.07 (s, 1H), 5.10 (s, 1H), 4.91 (q, |
| | | yl)phenyl]ethyl]pyrrolidine-2- | | J = 7.0 Hz, 1H), 4.37 (t, J = 7.9 Hz, |
| | | carboxamide | | 1H), 4.28 (s, 1H), 4.19 (d, J = 11.3 |
| | | | | Hz, 4H), 3.74-3.60 (m, 4H), 3.45- |
| | | | | 3.40 (m, 2H), 2.82 (t, J = 5.6 Hz, |
| | | | | 2H), 2.45 (d, J = 1.5 Hz, 3H), 2.28- |
| | | | | 2.15 (m, 1H), 2.10-1.97 (m, 1H), |
| | | | | 1.77 (ddd, J = 12.7, 8.0, 4.7 Hz, |
| | | | | 1H), 1.37 (d, J = 7.0 Hz, 3H), 1.24 |
| | | | | (s, 1H), 0.95 (t, J = 6.5 Hz, 3H), |
| | | | | 0.80 (dd, J = 14.7, 6.7 Hz, 3H). |
| 284 | B | (2S,4R)-4-hydroxy-1-[(2R)-2- | 869.58 | ¹H NMR (400 MHz, DMSO-d6) δ |
| | | [3-(2-[[(4-[3-(2- | | 14.14 (s, 1H), 8.98 (s, 1H), 8.98 (s, |
| | | hydroxyphenyl)-7H- | | 1H), 8.52 (d, J = 5.2 Hz, 1H), 8.40 |
| | | pyrrolo[2,3-c]pyridazin-6- | | (d, J = 5.7 Hz, 1H), 7.99 (dd, J = |
| | | yl]methyl]phenyl)methyl](methyl)ami- | | 8.1, 1.5 Hz, 1H), 7.48-7.41 (m, |
| | | no]ethoxy)-1,2-oxazol-5-yl]-3- | | 2H), 7.36 (d, J = 8.1 Hz, 2H), 7.29 |
| | | methylbutanoyl]-N-[(1S)-1- | | (q, J = 8.1 Hz, 4H), 6.98-6.89 (m, |
| | | [4-(4-methyl-1,3-thiazol-5- | | 2H), 6.34 (d, J = 4.8 Hz, 2H), 4.90 |
| | | yl)phenyl]ethyl]pyrrolidine-2- | | (t, J = 7.1 Hz, 2H), 4.37 (t, J = 7.9 |
| | | carboxamide | | Hz, 1H), 4.25 (t, J = 5.7 Hz, 3H), |
| | | | | 4.20 (s, 2H), 4.03 (s, 1H), 3.69 (dd, |
| | | | | J = 10.6, 4.3 Hz, 1H), 3.63 (d, J = |
| | | | | 9.7 Hz, 1H), 3.52 (d, J = 10.2 Hz, |
| | | | | 2H), 3.47-3.43 (m, 1H), 2.72 (t, |
| | | | | J = 5.7 Hz, 2H), 2.45 (s, 3H), 2.21- |
| | | | | 2.17 (m, 4H), 2.01 (d, J = 9.8 Hz, |
| | | | | 1H), 1.76 (d, J = 8.3 Hz, 1H), 1.40 |
| | | | | (dd, J = 27.0, 7.0 Hz, 3H), 0.95 (t, |
| | | | | J = 6.3 Hz, 3H), 0.80 (dd, J = 14.5, |
| | | | | 6.7 Hz, 3H) |
| 244 | B | (2S,4R)-4-hydroxy-1-((R)-2- | 819.4 | ¹H NMR (300 MHz, DMSO-d6) δ |
| | | (3-(2-(3-(2-(3-(2- | | 14.24 (s, 1H), 12.54 (s, 1H), 8.99 (s, |
| | | hydroxyphenyl)-7H- | | 1H), 8.53 (d, J = 3.4 Hz, 1H), 8.42 |
| | | pyrrolo[2,3-c]pyridazin-6- | | (d, J = 7.9 Hz, 1H), 8.03 (d, J = 8.0 |
| | | yl)ethyl)azetidin-1- | | Hz, 1H), 7.44 (d, J = 8.2 Hz, 2H), |
| | | yl)ethoxy)isoxazol-5-yl)-3- | | 7.37 (d, J = 8.1 Hz, 2H), 7.30 (t, |
| | | methylbutanoyl)-N-((S)-1-(4- | | J =8.28 Hz, 1H), 6.97 (d, J = 7.9 Hz, |
| | | (4-methylthiazol-5- | | 2H), 6.43 (s, 1H), 6.04 (s, 1H), 5.10 |
| | | yl)phenyl)ethyl)pyrrolidine-2- | | (d, J = 3.7 Hz, 1H), 4.91 (t, J = 7.0 |
| | | carboxamide | | Hz, 1H), 4.37 (t, J = 7.9 Hz, 1H), |
| | | | | 4.28 (s, 1H), 4.07 (t, J = 5.3 Hz, |
| | | | | 2H), 3.67 (m, J = 18.1, 8.1 Hz, 2H), |
| | | | | 3.54-3.37 (m, 2H), 2.97-2.62 (m, |
| | | | | 5H), 2.46 (s, 3H), 2.37 (m, 2H), |
| | | | | 2.20 (m, 1H), 2.00 (t, J = 7.7 Hz, |
| | | | | 3H), 1.78 (s, 1H), 1.42 (m, J = 21.6, |
| | | | | 7.0 Hz, 3H), 1.24 (s, 1H), 0.95 (d, |
| | | | | J = 6.3 Hz, 3H), 0.81 (m, J = 11.6, 6.7 |
| | | | | Hz, 4H) |
| 243 | B | (2S,4R)-4-hydroxy-1-[(2R)-2- | 833.3 | ¹H NMR (400 MHz, Methanol-d4) δ |
| | | {3-[2-(3-{3-[3-(2- | | 8.86 (d, J = 7.4 Hz, 1H), 8.38 (d, J = |
| | | hydroxyphenyl)-7H- | | 10.9 Hz, 1H), 7.96-7.88 (m, 1H), |
| | | pyrrolo[2,3-c]pyridazin-6- | | 7.47-7.34 (m, 4H), 7.31-7.23 (m, |
| | | yl]propyl}azetidin-1- | | 1H), 7.00-6.95 (m, 2H), 6.41 (s, |
| | | yl)ethoxy]-1,2-oxazol-5-yl}-3- | | 1H), 5.99 (s, 1H), 5.02 (q, J = 7.0 |
| | | methylbutanoyl]-N-[(1S)-1- | | Hz, 1H), 4.61 (s, 1H), 4.50 (t, J = |
| | | [4-(4-methyl-1,3-thiazol-5- | | 8.2 Hz, 1H), 4.46-4.36 (m, 1H), |
| | | yl)phenyl]ethyl]pyrrolidine-2- | | 4.26-4.16 (m, 2H), 3.82 (dd, J = |
| | | carboxamide | | 10.9, 4.1 Hz, 1H), 3.72-3.51 (m, |
| | | | | 4H), 2.96 (t, J = 7.5 Hz, 2H), 2.93- |
| | | | | 2.81 (m, 4H), 2.56 (p, J = 7.4 Hz, |
| | | | | 1H), 2.46 (s, 3H), 2.43-2.30 (m, |
| | | | | 1H), 2.22-2.13 (m, 1H), 2.01- |
| | | | | 1.88 (m, 1H), 1.82-1.71 (m, 2H), |
| | | | | 1.66 (q, J = 7.4 Hz, 2H), 1.51 (d, J = |
| | | | | 7.0 Hz, 3H), 1.04 (d, J = 6.5 Hz, |
| | | | | 3H), 0.87 (d, J = 6.7 Hz, 3H) |
| 238 | B | (2S,4R)-1-[(2R)-2-[3-(2-{3- | 825.38 | ¹H NMR (300 MHz, DMSO-d⁴) δ |
| | | [5-chloro-3-(2- | | 13.64 (s, 1H), 9.06 (s, 1H), 8.61- |
| | | hydroxyphenyl)-7H- | | 8.43 (m, 2H), 8.21 (dd, J = 8.2, 1.7 |
| | | pyrrolo[2,3-c]pyridazin-6- | | Hz, 1H), 7.55-7.48 (m, 2H), 7.47- |
| | | yl]azetidin-1-yl}ethoxy)-1,2- | | 7.34 (m, 3H), 7.12-6.96 (m, 2H), |
| | | oxazol-5-yl]-3- | | 6.09 (d, J = 48.9 Hz, 1H), 5.13 (d, |
| | | methylbutanoyl]-4-hydroxy- | | J = 28.8 Hz, 1H), 4.98 (t, J = 7.2 Hz, |
| | | N-[(1S)-1-[4-(4-methyl-1,3- | | 1H), 4.45 (t, J = 7.9 Hz, 1H), 4.36 |
| | | thiazol-5- | | (s, 1H), 4.25 (t, J = 5.3 Hz, 2H), |
| | | yl)phenyl]ethyl]pyrrolidine-2- | | 4.19-4.09 (m, 1H), 3.86 (t, J = 7.2 |
| | | carboxamide | | Hz, 2H), 3.81-3.69 (m, 2H), 3.65- |
| | | | | 3.54 (m, 2H), 3.53-3.47 (m, 1H), |
| | | | | 2.98 (t, J = 5.4 Hz, 2H), 2.53 (d, J = |
| | | | | 1.7 Hz, 3H), 2.38-2.29 (m, 1H), |
| | | | | 2.17-2.05 (m, 1H), 1.92-1.78 (m, |
| | | | | 1H), 1.56-1.41 (m, 3H), 1.10- |
| | | | | 0.99 (m, 3H), 0.96-0.82 (m, 3H) |
| 219 | B | (2S,4R)-4-hydroxy-1-((R)-2- | 792.25 | ¹H NMR (300 MHz, DMSO-d6) δ |
| | | (3-(2-(3-(3-(2- | | 14.22 (s, 1H), 12.66 (s, 1H), 9.07 (s, |
| | | hydroxyphenyl)-7H-pyrrolo | | 1H), 8.67-8.52 (m, 3H), 8.24- |
| | | [2,3-c]pyridazin-6-yl)azetidin- | | 8.05 (m, 1H), 7.90-7.82 (m, 1H), |
| | | 1-yl)ethoxy)isoxazol-5-yl)-3- | | 7.77 (d, J = 8.3 Hz, 1H), 7.51-7.29 |
| | | methylbutanoyl)-N-((S)-1-(6- | | (m, 2H), 7.04 (d, J = 7.8 Hz, 2H), |
| | | (4-methylthiazol-5-yl)pyridin- | | 6.64 (s, 1H), 6.17 (s, 1H), 5.18 (d, |
| | | 3-yl)ethyl)pyrrolidine-2- | | J = 3.6 Hz, 1H), 5.11-4.95 (m, 1H), |
| | | carboxamide | | 4.49-4.39 (m, 1H), 4.39-4.31 (m, |
| | | | | 1H), 4.29-4.18 (m, 2H), 4.05- |
| | | | | 3.92 (m, 1H), 3.91-3.65 (m, 3H), |
| | | | | 3.59-3.46 (m, 3H), 2.95 (s, 2H), |
| | | | | 2.72 (s, 3H), 2.32-2.02 (m, 2H), |
| | | | | 1.97-1.67 (m, 1H), 1.52 (dd, J = |
| | | | | 22.9, 7.0 Hz, 3H), 1.03 (d, J = 6.4 |
| | | | | Hz, 3H), 0.95-0.82 (m, 3H) |
| 218 | B | (2S,4R)-4-hydroxy-1-((S)-2- | 792.45 | ¹H NMR (300 MHz, DMSO-d6) δ |
| | | (3-(2-(3-(3-(2- | | 14.23 (s, 1H), 12.62 (s, 1H), 9.07 |
| | | hydroxyphenyl)-7H- | | (d, J = 8.2 Hz, 1H), 8.72-8.40 (m, |
| | | pyrrolo[2,3-c]pyridazin-6- | | 3H), 8.10 (d, J = 7.8 Hz, 1H), 7.98- |
| | | yl)azetidin-1- | | 7.67 (m, 2H), 7.54-7.20 (m, 2H), |
| | | yl)ethoxy)isoxazol-5-yl)-3- | | 7.04 (d, J = 8.2 Hz, 2H), 6.62 (d, J = |
| | | methylbutanoyl)-N-((S)-1-(6- | | 7.9 Hz, 1H), 6.18 (d, J = 15.4 Hz, |
| | | (4-methylthiazol-5-yl)pyridin- | | 1H), 5.26-4.90 (m, 2H), 4.71- |
| | | 3-yl)ethyl)pyrrolidine-2- | | 4.42 (m, 1H), 4.33 (s, 1H), 4.28- |
| | | carboxamide | | 4.14 (m, 2H), 4.03-3.88 (m, 1H), |
| | | | | 3.88-3.71 (m, 3H), 3.68-3.60 (m, |
| | | | | 1H), 3.59-3.50 (m, 1H), 3.50- |
| | | | | 3.46 (m, 1H), 2.98-2.86 (m, 2H), |
| | | | | 2.85-2.77 (m, 2H), 2.72 (d, J = 5.6 |
| | | | | Hz, 3H), 2.18-2.04 (m, 1H), 1.91- |
| | | | | 1.77 (m, 1H), 1.56 (d, J = 7.1 Hz, |
| | | | | 1H), 1.50-1.40 (m, 3H), 1.04 (d, |
| | | | | J = 6.6 Hz, 3H), 0.90 (d, J = 6.4 Hz, |
| | | | | 3H) |
| 217 | B | (2S,4R)-4-hydroxy-1-((R)-2- | 858.39 | ¹H NMR (300 MHz, DMSO-d6) δ |
| | | (3-(2-((6-(3-(2- | | 13.17 ( br s, 1H), 9.81 (br s, 1H), |
| | | hydroxyphenyl)-7H- | | 9.00 (s, 1H), 8.55 (s, 1H), 8.40 (d, |
| | | pyrrolo[2,3-c]pyridazin-6- | | J = 7.7 Hz, 1H), 7.77 (d, J = 7.6 Hz, |
| | | yl)spiro[3.3]heptan-2- | | 1H), 7.48-7.32 (m, 5H), 7.08- |
| | | yl)amino)ethoxy)isoxazol-5- | | 6.97 (m, 2H), 6.71 (s, 1H), 6.20 (d, |
| | | yl)-3-methylbutanoyl)-N-((S)- | | J = 3.4 Hz, 1H), 4.92 (t, J = 7.2 Hz, |
| | | 1-(4-(4-methylthiazol-5- | | 1H), 4.75-4.42 (m, 3H), 4.35 (dd, |
| | | yl)phenyl)ethyl)pyrrolidine-2- | | J = 16.4, 8.2 Hz, 4H), 3.84-3.61 |
| | | carboxamide | | (m, 4H), 3.62-3.26 (m, 3H), 3.17 |
| | | | | (s, 1H), 2.75-2.69 (m, 3H), 2.68- |
| | | | | 2.57 (m, 2H), 2.46 (s, 3H), 2.40- |
| | | | | 2.27 (m, 1H), 2.26-2.15 (m, 1H), |
| | | | | 2.14-1.96 (m, 2H), 1.87-1.72 (m, |
| | | | | 1H), 1.52-1.33 (m, 3H), 0.98 (d, |
| | | | | J = 7.0 Hz, 3H), 0.89-0.76 (m, 3H) |
| 208 | B | (2S,4R)-4-hydroxy-1-[(2R)- | 777.40 | ¹H NMR (300 MHz, DMSO-d6) δ |
| | | 2-[3-(2-{3-[3-(2- | | 14.17 (s, 1H), 12.55 (s, 1H), 8.98 |
| | | hydroxyphenyl)-7H-pyrrolo | | (d, J = 4.5 Hz, 1H), 8.58-8.50 (m, |
| | | [2,3-c]pyridazin-6-yl]azetidin- | | 2H), 8.03 (d, J = 8.1 Hz, 1H), 7.41 |
| | | 1-yl}ethoxy)-1,2-oxazol-5- | | (q, J = 8.1 Hz, 4H), 7.35-7.25 (m, |
| | | yl]-3-methylbutanoyl]-N-{[4- | | 1H), 6.96 (t, J = 7.9 Hz, 2H), 6.56 |
| | | (4-methyl-1,3-thiazol-5- | | (d, J = 5.1 Hz, 1H), 5.92 (d, J = |
| | | yl)phenyl]methyl}pyrrolidine- | | 111.0 Hz, 1H), 5.18-5.01 (m, 1H), |
| | | 2-carboxamide | | 4.46-4.29 (m, 4H), 4.16 (t, J = 5.3 |
| | | | | Hz, 2H), 3.89 (q, J = 7.4 Hz, 1H), |
| | | | | 3.82-3.65 (m, 4H), 3.54-3.35 (m, |
| | | | | 3H), 2.86 (t, J = 5.5 Hz, 2H), 2.46 |
| | | | | (s, 3H), 2.42 (s, 1H), 2.10-1.99 |
| | | | | (m, 1H), 1.98-1.84 (m, 1H), 0.95 |
| | | | | (d, J = 6.5 Hz, 3H), 0.81 (d, J = 6.6 |
| | | | | Hz, 3H). |
| 198 | B | (2S,4R)-4-hydroxy-1-((R)-2- | 833.3 | ¹H NMR (400 MHz, DMSO-d6) δ |
| | | (3-(2-(3-(2-(3-(2- | | 14.43 (s, 1H), 12.23 (s, 1H), 8.99 (s, |
| | | hydroxyphenyl)-5-methyl- | | 1H), 8.54 (s, 1H), 8.42 (d, J = 7.6 |
| | | 7H-pyrrolo[2,3-c]pyridazin-6- | | Hz, 1H), 8.14-8.05 (m, 1H), 7.50- |
| | | yl)ethyl)azetidin-1- | | 7.23 (m, 5H), 7.00-6.90 (m, 2H), |
| | | yl)ethoxy)isoxazol-5-yl)-3- | | 6.02 (s, 1H), 5.10 (d, J = 3.6 Hz, |
| | | methylbutanoyl)-N-((S)-1-(4- | | 1H), 4.97-4.85 (m, 1H), 4.43- |
| | | (4-methylthiazol-5- | | 4.22 (m, 2H), 4.05 (t, J = 5.4 Hz, |
| | | yl)phenyl)ethyl)pyrrolidine-2- | | 2H), 3.75-3.60 (m, 2H), 3.48- |
| | | carboxamide | | 3.40 (m, 1H), 2.84-2.63 (m, 6H), |
| | | | | 2.46 (s, 3H), 2.41-2.12 (m, 6H), |
| | | | | 2.10-1.98 (m, 1H), 1.97-1.88 (m, |
| | | | | 2H), 1.83-1.72 (m, 1H), 1.37 (d, J = |
| | | | | 7.0 Hz, 3H), 0.95 (d, J = 6.6 Hz, |
| | | | | 3H), 0.78 (d, J = 6.7 Hz, 3H) |
| 185 | B | (2S,4R)-4-hydroxy-1-[(2R)-2- | 821.5 | ¹H NMR (400 MHz, Methanol-d4) δ |
| | | (3-{2-[3-{{[3-(2- | | 8.86 (d, J = 7.5 Hz, 1H), 8.51 (d, J = |
| | | hydroxyphenyl)-7H- | | 18.9 Hz, 2H), 7.96 (t, J = 8.0 Hz, |
| | | pyrrolo[2,3-c]pyridazin-5- | | 1H), 7.52-7.37 (m, 4H), 7.30 (dd, |
| | | yl]oxy}methyl)azetidin-1- | | J = 20.5, 7.8 Hz, 1H), 7.10-6.84 |
| | | yl]ethoxy}-1,2-oxazol-5-yl)-3- | | (m, 2H), 6.01 (d, J = 17.7 Hz, 1H), |
| | | methylbutanoyl]-N-[(1S)-1- | | 5.02 (d, J = 7.0 Hz, 1H), 4.49 (t, J = |
| | | [4-(4-methyl-1,3-thiazol-5- | | 8.2 Hz, 1H), 4.42 (s, 1H), 4.34 (s, |
| | | yl)phenyl]ethyl]pyrrolidine-2- | | 1H), 4.25 (d, J = 5.7 Hz, 2H), 3.94- |
| | | carboxamide | | 3.75 (m, 4H), 3.61-3.58 (m, 4H), |
| | | | | 3.74-3.53 (m, 3H), 3.19 (s, 3H), |
| | | | | 2.35 (t, J = 14.7 Hz, 1H), 2.25- |
| | | | | 2.10 (m, 1H), 2.07-1.83 (m, 1H), |
| | | | | 1.53 (dd, J = 20.1, 7.0 Hz, 3H), 1.03 |
| | | | | (d, J = 6.6 Hz, 3H), 0.86 (d, J = 6.9 |
| | | | | Hz, 3H) |
| 164 | B | (2S,4R)-1-((R)-2-(3-(2-(3-(5- | 831.70 | ¹H NMR (400 MHz, DMSO-d6) δ |
| | | cyclopropyl-3-(2- | | 14.08 (d, J = 5.1 Hz, 1H), 12.50 (s, |
| | | hydroxyphenyl)-7H- | | 1H), 8.98 (d, J = 2.1 Hz, 1H), 8.41 |
| | | pyrrolo[2,3-c]pyridazin-6- | | (d, J = 7.6 Hz, 1H), 8.36 (d, J = 3.5 |
| | | yl)azetidin-1- | | Hz, 1H), 8.12-8.05 (m, 1H), 7.44 |
| | | yl)ethoxy)isoxazol-5-yl)-3- | | (d, J = 8.2 Hz, 2H), 7.36 (d, J = 8.2 |
| | | methylbutanoyl)-4-hydroxy- | | Hz, 2H), 7.29 (t, J = 7.7 Hz, 1H), |
| | | N-((S)-1-(4-(4-methylthiazol- | | 6.99-6.91 (m, 2H), 6.09 (s, 1H), |
| | | 5-yl)phenyl)ethyl)pyrrolidine- | | 5.11 (d, J = 3.6 Hz, 1H), 4.92 (q, J = |
| | | 2-carboxamide | | 6.9 Hz, 1H), 4.37 (t, J = 7.9 Hz, 1H), |
| | | | | 4.29 (s, 1H), 4.18 (d, J = 6.1 Hz, |
| | | | | 3H), 3.78 (t, J = 7.3 Hz, 2H), 3.72- |
| | | | | 3.62 (m, 2H), 3.47 (s, 3H), 2.90 (t, |
| | | | | J = 5.5 Hz, 2H), 2.45 (d, J = 2.8 Hz, |
| | | | | 3H), 2.13 (t, J = 10.2 Hz, 2H), 1.78 |
| | | | | (dq, J = 12.5, 7.3, 6.0 Hz, 2H), 1.45 |
| | | | | (d, J = 7.0 Hz, 1H), 1.38 (d, J = 7.0 |
| | | | | Hz, 2H), 0.97 (t, J = 6.1 Hz, 5H), |
| | | | | 0.82 (dd, J = 13.8, 6.7 Hz, 3H), 0.68 |
| | | | | (td, J = 5.8, 4.1 Hz, 2H) |
| 152 | B | (2S,4R)-4-hydroxy-1-[(2R)-2- | 775.45 | ¹H NMR (400 MHz, Methanol-d⁴) δ |
| | | [3-(2-{3-[3-(2- | | 8.87 (s, 1H), 8.44 (s, 1H), 7.96- |
| | | hydroxyphenyl)-7H- | | 7.91 (m, 1H), 7.46-7.38 (m, 3H), |
| | | pyrrolo[2,3-c]pyridazin-6- | | 7.31-7.23 (m, 2H), 7.00-6.90 (m, |
| | | yllazetidin-1-yl}ethyl)-1,2- | | 2H), 6.57 (d, J = 0.8 Hz, 1H), 6.34 |
| | | oxazol-5-yl]-3- | | (s, 1H), 5.07-4.99 (m, 1H), 4.50 (t, |
| | | methylbutanoyl]-N-[(1S)-1- | | J = 8.2 Hz, 1H), 4.46-4.40 (m, |
| | | [4-(4-methyl-1,3-thiazol-5- | | 1H), 4.00-3.91 (m, 1H), 3.88- |
| | | yl)phenyl]ethyl]pyrrolidine-2- | | 3.75 (m, 4H), 3.66-3.59 (m, 1H), |
| | | carboxamide | | 3.50-3.36 (m, 2H), 2.90 (t, J = 7.2 |
| | | | | Hz, 2H), 2.77 (t, J = 7.2 Hz, 2H), |
| | | | | 2.45-2.32 (m, 4H), 2.20-2.06 (m, |
| | | | | 1H), 1.99-1.90 (m, 1H), 1.54 (d, |
| | | | | J = 7.1 Hz, 3H), 1.07 (d, J = 6.7 Hz, |
| | | | | 3H), 0.88 (d, J = 6.7 Hz, 3H) |
| 139 | B | (2S,4R)-4-hydroxy-1-[(2R)-2- | 807.5 | ¹H NMR (300 MHz, DMSO-d6) δ |
| | | {3-[2-(3-{[3-(2- | | 13.79 (s, 1H), 12.20 (s, 1H), 9.06 (s, |
| | | hydroxyphenyl)-7H- | | 1H), 8.64 (s, 1H), 8.48 (d, J = 7.6 |
| | | pyrrolo[2,3-c]pyridazin-5- | | Hz, 1H), 8.20 (d, J = 7.5 Hz, 1H), |
| | | yl]oxy}azetidin-1-yl)ethoxy]- | | 7.67 (s, 1H), 7.51 (d, J = 8.3 Hz, |
| | | 1,2-oxazol-5-yl}-3- | | 2H), 7.44 (d, J = 8.3 Hz, 2H), 7.36 |
| | | methylbutanoyl]-N-[(1S)-1- | | (t, J = 7.6 Hz, 1H), 7.08-6.96 (m, |
| | | [4-(4-methyl-1,3-thiazol-5- | | 2H), 6.16 (s, 1H), 5.17 (d, J = 3.7 |
| | | yl)phenyl]ethyl]pyrrolidine-2- | | Hz, 1H), 5.04-4.88 (m, 2H), 4.44 |
| | | carboxamide | | (t, J = 7.8 Hz, 1H), 4.36 (s, 1H), |
| | | | | 4.22 (t, J = 5.3 Hz, 2H), 3.90 (t, J = |
| | | | | 7.1 Hz, 2H), 3.75 (m, J = 15.6, 8.0 |
| | | | | Hz, 2H), 3.53 (s, 1H), 3.27 (t, J = |
| | | | | 6.7 Hz, 2H), 2.92 (t, J = 5.2 Hz, 2H), |
| | | | | 2.53 (d, J = 3.0 Hz, 3H), 2.40-2.33 |
| | | | | (m, 1H), 2.31 (s, 1H), 2.28 (s, 1H), |
| | | | | 1.49 (m, J = 21.1, 7.1 Hz, 3H), 1.03 |
| | | | | (d, J = 6.5 Hz, 3H), 0.87 (d, J = 6.7 |
| | | | | Hz, 3H). |
| 137 | B | (2S,4R)-4-hydroxy-1-[(2R)-2- | 761.2 | ¹H NMR (400 MHz, Methanol-d4) δ |
| | | [3-({3-[3-(2-hydroxyphenyl)- | | 8.87 (s, 1H), 8.62 (d, J = 7.5 Hz, |
| | | 7H-pyrrolo[2,3-c]pyridazin-6- | | 1H), 8.53 (s, 1H), 7.83 (d, J = 7.5 |
| | | yl]azetidin-1-yl}methyl)-1,2- | | Hz, 1H), 7.52-7.27 (m, 5H), 7.06- |
| | | oxazol-5-yl]-3- | | 6.96 (m, 2H), 6.82 (s, 1H), 6.47 (s, |
| | | methylbutanoyl]-N-[(1S)-1- | | 1H), 5.04 (t, J = 6.9 Hz, 1H), 4.53- |
| | | [4-(4-methyl-1,3-thiazol-5- | | 4.15 (m, 9H), 3.93-3.81 (m, 2H), |
| | | yl)phenyl]ethyl]pyrrolidine-2- | | 3.64 (d, J = 10.4 Hz, 1H), 2.50- |
| | | carboxamide | | 2.38 (m, 4H), 2.24-2.14 (m, 1H), |
| | | | | 2.01-1.90 (m, 1H), 1.52 (d, J = 7.0 |
| | | | | Hz, 3H), 1.08 (d, J = 6.5 Hz, 3H), |
| | | | | 0.89-0.84 (m, 3H) |
| 90 | B | (6R*)-6-({2-[(5-{1-[(2S,4R)-4- | 849.20 | ¹H NMR (400 MHz, DMSO-d⁶) δ |
| | | hydroxy-2-{[(1S)-1-[4-(4- | | 14.62-14.44 (m, 1H), 8.98 (s, 1H), |
| | | methyl-1,3-thiazol-5- | | 8.50 (m, 1H), 8.46-8.37 (m, 2H), |
| | | yl)phenyl]ethyl]carbamoyl}pyrrolidin- | | 8.15-8.05 (m, 1H), 7.47-7.40 (m, |
| | | 1-yl]-3-methyl-1- | | 2H), 7.40-7.33 (m, 2H), 7.33- |
| | | oxobutan-2-yl}-1,2-oxazol-3- | | 7.22 (m, 2H), 6.99-6.89 (m, 2H), |
| | | yl)oxy]ethyl}amino)-3-(2- | | 6.09-6.03 (m, 1H), 4.97-4.81 (m, |
| | | hydroxyphenyl)- | | 1H), 4.45-4.32 (m, 2H), 4.30- |
| | | 5H,7H,8H,9H-pyridazino[3,4- | | 4.21 (m, 1H), 4.21-4.10 (m, 2H), |
| | | b]indole-6-carboxylic acid | | 3.81-3.43 (m, 4H), 3.20-3.00 (m, |
| | | | | 1H), 2.95-2.70 (m, 4H), 2.45 (s, |
| | | | | 3H), 2.28-1.90 (m, 3H), 1.85- |
| | | | | 1.68 (m, 2H), 1.35 (d, J = 7.0 Hz, |
| | | | | 3H), 0.94 (d, J = 6.6 Hz, 3H), 0.78 |
| | | | | (m, J = 6.6 Hz, 3H). |
| 119 | B | (2S,4R)-N-((S)-1-(2′-fluoro- | 788.3 | ¹H NMR (300 MHz, Methanol-d4) δ |
| | | [1,1′-biphenyl]-4-yl)ethyl)-4- | | 8.46 (s, 1H), 8.04-7.85 (m, 1H), |
| | | hydroxy-1-((R)-2-(3-(2-(3-(3- | | 7.56-7.43 (m, 2H), 7.41-7.11 (m, |
| | | (2-hydroxyphenyl)-7H- | | 7H), 7.06-6.91 (m, 2H), 6.61 (s, |
| | | pyrrolo[2,3-c]pyridazin-6- | | 1H), 6.05 (s, 1H), 5.11-4.99 (m, |
| | | yl)azetidin-1- | | 1H), 4.53 (t, J = 8.2 Hz, 1H), 4.49- |
| | | yl)ethoxy)isoxazol-5-yl)-3- | | 4.40 (m, 1H), 4.31 (t, J = 5.2 Hz, |
| | | methylbutanoyl)pyrrolidine- | | 2H), 4.09-3.80 (m, 4H), 3.75- |
| | | 2-carboxamide | | 3.49 (m, 4H), 3.02 (t, J = 5.2 Hz, |
| | | | | 2H), 2.48-2.29 (m, 1H), 2.27- |
| | | | | 2.13 (m, 1H), 2.09-1.90 (m, 1H), |
| | | | | 1.54 (d, J = 7.0 Hz, 3H), 1.08 (d, J = |
| | | | | 6.5 Hz, 3H), 0.91 (d, J = 6.8 Hz, |
| | | | | 3H). |
| 120 | B | (2S,4R)-1-[(2R)-2-[3-(2-{3- | 869.24 | ¹H NMR (400 MHz, Methanol-d4) δ |
| | | [5-bromo-3-(2- | | 8.82 (d, J = 38.9 Hz, 1H), 8.19 (d, |
| | | hydroxyphenyl)-7H- | | J = 53.5 Hz, 1H), 8.05-7.78 (m, 1H), |
| | | pyrrolo[2,3-c]pyridazin-6- | | 7.53-7.37 (m, 3H), 7.36-7.15 (m, |
| | | yllazetidin-1-yl}ethoxy)-1,2- | | 2H), 7.09-6.88 (m, 2H), 6.00 (d, |
| | | oxazol-5-yl]-3- | | J = 31.2 Hz, 1H), 5.03 (q, J = 6.9 Hz, |
| | | methylbutanoyl]-4-hydroxy- | | 1H), 4.51 (t, J = 8.2 Hz, 1H), 4.43 |
| | | N-[(1S)-1-[4-(4-methyl-1,3- | | (s, 1H), 4.30 (t, J = 5.1 Hz, 1H), |
| | | thiazol-5- | | 4.26-4.12 (m, 1H), 3.95 (q, J = 7.7 |
| | | yl)phenyl]ethyl]pyrrolidine-2- | | Hz, 2H), 3.83 (dd, J = 10.9, 4.1 Hz, |
| | | carboxamide | | 1H), 3.74 (d, J = 12.3 Hz, 1H), 3.73- |
| | | | | 3.58 (m, 3H), 3.57-3.45 (m, 1H), |
| | | | | 3.16-2.94 (m, 2H), 2.47 (s, 2H), |
| | | | | 2.45-2.30 (m, 2H), 2.23-2.10 (m, |
| | | | | 1H), 2.04-1.80 (m, 1H), 1.54 (dd, |
| | | | | J = 22.4, 7.0 Hz, 3H), 1.05 (dd, J = |
| | | | | 6.6, 1.9 Hz, 3H), 0.90 (dd, J = 11.7, |
| | | | | 6.7 Hz, 3H). |
| 121 | B | (2S,4R)-1-[(2R)-2-[3-(2-{3- | 809.32 | ¹H NMR (400 MHz, Methanol-d4) δ |
| | | [5-fluoro-3-(2- | | 8.90 (d, J = 12.8 Hz, 1H), 8.64 (d, |
| | | hydroxyphenyl)-7H- | | J = 24.0 Hz, 1H), 7.72 (dd, J = 15.8, |
| | | pyrrolo[2,3-c]pyridazin-6- | | 8.2 Hz, 1H), 7.49-7.33 (m, 5H), |
| | | yl]azetidin-1-yl}ethoxy)-1,2- | | 7.08 (t, J = 7.8 Hz, 2H), 6.08 (d, J = |
| | | oxazol-5-yl]-3- | | 20.9 Hz, 1H), 5.06-4.99 (m, 1H), |
| | | methylbutanoyl]-4-hydroxy- | | 4.74 (d, J = 8.6 Hz, 4H), 4.55 (t, J = |
| | | N-[(1S)-1-[4-(4-methyl-1,3- | | 4.6 Hz, 2H), 4.50 (t, J = 8.2 Hz, 1H), |
| | | thiazol-5- | | 4.47-4.39 (m, 1H), 3.95-3.79 (m, |
| | | yl)phenyl]ethyl]pyrrolidine-2- | | 2H), 3.78-3.58 (m, 3H), 3.49 (dd, |
| | | carboxamide | | J = 11.1, 5.3 Hz, 1H), 2.46 (d, J = |
| | | | | 12.7 Hz, 3H), 2.42-2.27 (m, 1H), |
| | | | | 2.19 (dd, J = 14.3, 7.0 Hz, 1H), 2.09- |
| | | | | 1.83 (m, 1H), 1.62 (dd, J = 23.1, |
| | | | | 7.0 Hz, 1H), 1.51 (d, J = 7.0 Hz, |
| | | | | 2H), 1.29 (s, 1H), 1.06 (d, J = 6.5 |
| | | | | Hz, 3H), 0.90 (t, J = 7.5 Hz, 3H) |
| 123 | B | (2S,4R)-4-hydroxy-1-[(2R)-2- | 776.31 | ¹H NMR (300 MHz, DMSO-d6) δ |
| | | (3-{2-[11-(2-hydroxyphenyl)- | | 14.30 (s, 1H), 12.68 (s, 1H), 9.09- |
| | | 4,7,9,10- | | 9.02 (m, 1H), 8.60 (s, 1H), 8.50 (d, |
| | | tetraazatricyclo[6.4.0.0\hat{ }{2,6}] | | J = 7.7 Hz, 1H), 8.16-8.05 (m, |
| | | dodeca-1(12),2(6),8,10- | | 1H), 7.54-7.49 (m, 2H), 7.47- |
| | | tetraen-4-yl]ethoxy}-1,2- | | 7.33 (m, 3H), 7.08-6.96 (m, 2H), |
| | | oxazol-5-yl)-3- | | 6.21 (s, 1H), 5.21-5.15 (m, 1H), |
| | | methylbutanoyl]-N-[(1S)-1- | | 4.99 (t, J = 7.2 Hz, 1H), 4.50-4.40 |
| | | [4-(4-methyl-1,3-thiazol-5- | | (m, 3H), 4.36 (s, 1H), 4.25 (s, 2H), |
| | | yl)phenyl]ethyl]pyrrolidine-2- | | 4.15 (s, 2H), 3.84-3.70 (m, 2H), |
| | | carboxamide | | 3.54 (d, J = 10.6 Hz, 1H), 3.32 (t, |
| | | | | J = 5.5 Hz, 2H), 2.53 (s, 3H), 2.33 (d, |
| | | | | J = 9.1 Hz, 1H), 2.41-2.21 (m, |
| | | | | 1H), 2.17-2.04 (m, 1H), 1.93- |
| | | | | 1.78 (m, 1H), 1.49 (dd, J = 23.4, 7.0 |
| | | | | Hz, 3H), 1.04 (d, J = 6.5 Hz, 3H), |
| | | | | 0.90 (dd, J = 10.5, 6.7 Hz, 3H). |
| 131 | B | (2S,4R)-1-[(2R)-2-(3-{2- | 851.15 | ¹H NMR (300 MHz, DMSO-d6) δ |
| | | [(1R,5S,6S)-6-[5-chloro-3-(2- | | 13.72 (s, 1H), 12.59 (s, 1H), 8.98 (s, |
| | | hydroxyphenyl)-7H- | | 1H), 8.46-8.37 (m, 2H), 8.14 (d, |
| | | pyrrolo[2,3-c]pyridazin-6-yl]- | | J = 7.9 Hz, 1H), 7.48-7.41 (m, 2H), |
| | | 3-azabicyclo[3.1.0]hexan-3- | | 7.41-7.33 (m, 2H), 7.32-7.26 (m, |
| | | yl]ethoxy}-1,2-oxazol-5-yl)-3- | | 1H), 7.01-6.92 (m, 2H), 6.14 (s, |
| | | methylbutanoyl]-4-hydroxy- | | 1H), 5.07 (d, J = 3.7 Hz, 1H), 4.99- |
| | | N-[(1S)-1-[4-(4-methyl-1,3- | | 4.84 (m, 1H), 4.38 (t, J = 7.9 Hz, |
| | | thiazol-5- | | 1H), 4.32-4.23 (m, 3H), 3.76- |
| | | yl)phenyl]ethyl]pyrrolidine-2- | | 3.52 (m, 2H), 3.53-3.39 (m, 1H), |
| | | carboxamide | | 3.25 (d, J = 9.3 Hz, 2H), 2.96-2.80 |
| | | | | (m, 2H), 2.64-2.54 (m, 3H), 2.46 |
| | | | | (s, 3H), 2.34-2.26 (m, 2H), 2.31- |
| | | | | 2.13 (m, 1H), 2.10-1.99 (m, 1H), |
| | | | | 1.98-1.71 (m, 1H), 1.43 (dd, J = |
| | | | | 7.0 Hz, 3H), 0.97 (d, J = 6.3 Hz, |
| | | | | 3H), 0.81 (d, J = 6.7 Hz, 3H). |
| 206 | B | (2S,4R)-4-hydroxy-1-[(2R)- | 808.2 | ¹H NMR (300 MHz, DMSO-d6) δ |
| | | 2-[3-(2-{3-[3-(2- | | 14.10 (s, 1H), 12.37 (s, 1H), 8.99 (s, |
| | | hydroxyphenyl)-6-oxo-7H- | | 1H), 8.86-8.33 (m, 2H), 7.90 (dd, |
| | | imidazo[4,5-c]pyridazin-5-yl] | | J = 8.1, 1.6 Hz, 1H), 7.49-7.41 (m, |
| | | azetidin-1-yl}ethoxy)-1,2- | | 2H), 7.40-7.25 (m, 3H), 7.01- |
| | | oxazol-5-yl]-3- | | 6.91 (m, 2H), 6.01 (d, J = 42.2 Hz, |
| | | methylbutanoyl]-N-[(1S)-1- | | 1H), 5.09 (s, 1H), 5.04-4.84 (m, |
| | | [4-(4-methyl-1,3-thiazol-5- | | 2H), 4.37 (t, J = 7.8 Hz, 1H), 4.31- |
| | | yl)phenyl]ethyl]pyrrolidine-2- | | 4.18 (m, 3H), 3.83-3.52 (m, 6H), |
| | | carboxamide | | 3.45 (dd, J = 10.1, 5.8 Hz, 1H), 2.95 |
| | | | | (t, J = 5.2 Hz, 2H), 2.46 (s, 3H), |
| | | | | 2.25-2.13 (m, 1H), 2.09-1.94 (m, |
| | | | | 1H), 1.85-1.73 (m, 1H), 1.41 (dd, |
| | | | | J = 17.5, 7.0 Hz, 3H), 0.96 (dd, J = |
| | | | | 6.6, 3.4 Hz, 3H), 0.79 (dd, J = 9.4, |
| | | | | 6.6 Hz, 3H) |
| 177 | B | (2S,4R)-4-hydroxy-1-((R)-2- | 779.6 | ¹H NMR (400 MHz, Methanol-d4) δ |
| | | (3-(2-(3′-(2-hydroxyphenyl)- | | 8.88 (s, 1H), 8.71-8.47 (m, 2H), |
| | | 6′,7′-dihydrospiro[azetidine- | | 7.84 (d, J = 7.9 Hz, 1H), 7.59-7.26 |
| | | 3,5′-pyrrolo[2,3-c]pyridazin]- | | (m, 5H), 6.98 (t, J = 8.4 Hz, 2H), |
| | | 1-yl)ethoxy)isoxazol-5-yl)-3- | | 6.09 (s, 1H), 5.05 (m, 1H), 4.68- |
| | | methylbutanoyl)-N-((S)-1-(4- | | 4.53 (m, 2H),4.51-4.50 (m, |
| | | (4-methylthiazol-5- | | 2H),4.49-4.35 (m, 3H), 4.11 (s, |
| | | yl)phenyl)ethyl)pyrrolidine-2- | | 2H), 3.92-3.55 (m, 6H), 2.47 (m, |
| | | carboxamide | | 3H), 2.38 (m, 1H), 2.20 (m,1H), |
| | | | | 1.90 (m,1H), 1.55 (dd, J = 30.1, 7.0 |
| | | | | Hz, 3H), 1.15 (m, 3H), 0.89 (t, J = |
| | | | | 7.5 Hz, 3H) |
| 148 | B | (2S,4R)-1-[(2R)-2-[3-(2-{3- | 841.4 | ¹H NMR (400 MHz, Methanol-d4) δ |
| | | [7-(difluoromethyl)-3-(2- | | 8.92-8.79 (m, 1H), 8.57 (d, J = |
| | | hydroxyphenyl)pyrrolo[2,3- | | 29.3 Hz, 1H), 8.32 (d, J = 57.8 Hz, |
| | | c]pyridazin-6-yl]azetidin-1- | | 1H), 7.76 (dd, J = 10.3, 8.6 Hz, 1H), |
| | | yl}ethoxy)-1,2-oxazol-5-yl]-3- | | 7.42 (p, J = 7.7, 6.9 Hz, 4H), 7.33 (t, |
| | | methylbutanoyl]-4-hydroxy- | | J = 9.5 Hz, 1H), 7.09-6.93 (m, |
| | | N-[(1S)-1-[4-(4-methyl-1,3- | | 2H), 6.75 (s, 1H), 6.07-5.93 (m, |
| | | thiazol-5- | | 1H), 5.03 (q, J = 7.1 Hz, 1H), 4.51 |
| | | yl)phenyl]ethyl]pyrrolidine-2- | | (t, J = 8.1 Hz, 1H), 4.41 (d, J = 16.7 |
| | | carboxamide | | Hz, 1H), 4.28 (t, J = 5.0 Hz, 2H), |
| | | | | 4.11-4.01 (m, 1H), 3.91 (t, J = 7.5 |
| | | | | Hz, 2H), 3.83 (dd, J = 10.8, 4.2 Hz, |
| | | | | 1H), 3.64 (dd, J = 27.0, 10.2 Hz, |
| | | | | 4H), 3.00 (d, J = 5.4 Hz, 2H), 2.55- |
| | | | | 2.41 (m, 3H), 2.36 (d, J = 9.4 Hz, |
| | | | | 1H), 2.17 (t, J = 10.8 Hz, 1H), 2.04- |
| | | | | 1.87 (m, 1H), 1.55 (dd, J = 25.2, |
| | | | | 6.9 Hz, 3H), 1.05 (d, J = 6.5 Hz, |
| | | | | 3H), 0.88 (d, J = 6.8 Hz, 3H) |
| 140 | B | (2S,4R)-4-hydroxy-1-((R)-2- | 805.60 | ¹H NMR (400 MHz, Methanol-d4) δ |
| | | (3-(2-(3-(3-(2- | | 8.85 (d, J = 15.1 Hz, 1H), 8.46 (s, |
| | | hydroxyphenyl)-7-methyl- | | 1H), 7.94 (d, J = 7.9 Hz, 1H), 7.48- |
| | | 7H-pyrrolo[2,3-c]pyridazin-6- | | 7.40 (m, 3H), 7.42-7.28 (m, 2H), |
| | | yl)azetidin-1- | | 6.97 (t, J = 7.5 Hz, 2H), 6.68-6.61 |
| | | yl)ethoxy)isoxazol-5-yl)-3- | | (m, 1H), 6.03 (s, 1H), 5.03 (d, J = |
| | | methylbutanoyl)-N-((S)-1-(4- | | 7.0 Hz, 1H), 4.50 (t, J = 8.2 Hz, 1H), |
| | | (4-methylthiazol-5- | | 4.43 (s, 1H), 4.28 (t, J = 5.1 Hz, |
| | | yl)phenyl)ethyl)pyrrolidine-2- | | 2H), 4.16-4.07 (m, 1H), 4.00 (t, |
| | | carboxamide | | J = 7.6 Hz, 2H), 3.90-3.79 (m, 4H), |
| | | | | 3.69 (t, J = 9.7 Hz, 1H), 3.61 (d, J = |
| | | | | 11.0 Hz, 1H), 3.52 (dt, J = 7.4, 4.4 |
| | | | | Hz, 2H), 2.97 (t, J = 5.1 Hz, 2H), |
| | | | | 2.48-2.29 (m, 3H), 2.37 (s, 1H), |
| | | | | 2.22-2.12 (m, 1H), 1.95 (ddd, J = |
| | | | | 13.4, 8.9, 4.7 Hz, 1H), 1.54 (dd, J = |
| | | | | 21.2, 7.0 Hz, 3H), 1.05 (d, J = 6.5 |
| | | | | Hz, 3H), 0.90 (t, J = 7.9 Hz, 3H) |
| 134 | B | (2S,4R)-4-hydroxy-1-[(2R)-2- | 807.00 | ¹H NMR (300 MHz, DMSO-d6) δ |
| | | [3-(2-{3-[3-(2- | | 14.30 (s, 1H), 9.06 (d, J = 1.4 Hz, |
| | | hydroxyphenyl)-5-methyl- | | 1H), 8.49 (d, J = 7.6 Hz, 1H), 8.16 |
| | | 6H,7H-pyrrolo[2,3- | | (s, 1H), 8.06-7.97 (m, 1H), 7.60- |
| | | c]pyridazin-5-ylazetidin-1- | | 7.39 (m, 5H), 7.33 (t, J = 7.7 Hz, |
| | | yl}ethoxy)-1,2-oxazol-5-yl]-3- | | 1H), 7.03-6.92 (m, 2H), 6.12 (s, |
| | | methylbutanoyl]-N-[(1S)-1- | | 1H), 5.17 (d, J = 3.6 Hz, 1H), 4.98 |
| | | [4-(4-methyl-1,3-thiazol-5- | | (t, J = 7.2 Hz, 1H), 4.43 (t, J = 7.9 |
| | | yl)phenyl]ethyl]pyrrolidine-2- | | Hz, 1H), 4.35 (brs, 1H), 4.12 (t, J = |
| | | carboxamide | | 5.4 Hz, 2H), 3.88-3.66 (m, 3H), |
| | | | | 3.51 (d, J = 10.4 Hz, 1H), 3.37- |
| | | | | 3.34 (m, 1H),3.28 (d, J = 7.7 Hz, |
| | | | | 1H), 3.08-2.87 (m, 4H), 2.75 (t, |
| | | | | J = 5.4 Hz, 2H), 2.53 (s, 3H), 2.34- |
| | | | | 2.23 (m, 1H), 2.11 (dd, J = 19.0, 7.6 |
| | | | | Hz, 1H), 1.92-1.77 (m, 1H), 1.55- |
| | | | | 1.38 (m, 6H), 1.02 (d, J = 6.5 Hz, |
| | | | | 3H), 0.85 (d, J = 6.7 Hz, 3H) |
| 122 | B | (2S,4R)-4-hydroxy-1-((2R)- | 807.3 | ¹H NMR (400 MHz, Methanol-d4) δ |
| | | 2-(3-(2-(3-(3-(2- | | 8.87 (s, 1H), 7.95 (s, 1H), 7.83- |
| | | hydroxyphenyl)-6-methyl- | | 7.68 (m, 1H), 7.53-7.31 (m, 4H), |
| | | 6,7-dihydro-5H-pyrrolo[2,3- | | 7.33-7.20 (m, 1H), 6.92 (t, J = 7.9 |
| | | c]pyridazin-6-yl)azetidin-1- | | Hz, 2H), 6.15-5.86 (m, 1H), 5.02 |
| | | yl)ethoxy)isoxazol-5-yl)-3- | | (q, J = 6.9 Hz, 1H), 4.57-4.24 (m, |
| | | methylbutanoyl)-N-((S)-1-(4- | | 4H), 4.02 (dt, J = 29.7, 9.3 Hz, 2H), |
| | | (4-methylthiazol-5- | | 3.91-3.56 (m, 5H), 3.54-3.42 (m, |
| | | yl)phenyl)ethyl)pyrrolidine-2- | | 1H), 3.38 (s, 1H), 3.17 (q, J = 8.8 |
| | | carboxamide | | Hz, 1H), 3.04 (d, J = 8.7 Hz, 2H), |
| | | | | 2.47 (s, 3H), 2.34 (td, J = 14.4, |
| | | | | 12.8, 5.6 Hz, 1H), 2.18 (dd, J = |
| | | | | 13.3, 7.9 Hz, 1H), 1.94 (ddd, J = |
| | | | | 13.3, 8.9, 4.5 Hz, 1H), 1.54 (dd, J = |
| | | | | 29.8, 7.0 Hz, 3H), 1.31 (d, J = 5.5 |
| | | | | Hz, 3H), 1.04 (dt, J = 6.4, 3.0 Hz, |
| | | | | 3H), 0.87 (dt, J = 11.7, 6.7 Hz, 3H) |
| 115 | B | (2S,4R)-4-hydroxy-1-((R)-2- | 765.25 | ¹H NMR (400 MHz, Methanol-d4) δ |
| | | (3-(2-(3-(3-(2- | | 8.65 (d, J = 8.6 Hz, 1H), 8.42 (s, |
| | | hydroxyphenyl)-7H- | | 1H), 7.92 (d, J = 7.8 Hz, 1H), 7.28 |
| | | pyrrolo[2,3-c]pyridazin-6- | | (t, J = 7.7 Hz, 1H), 6.96 (t, J = 7.9 |
| | | yl)azetidin-1- | | Hz, 2H), 6.59 (s, 1H), 6.01 (d, J = |
| | | yl)ethoxy)isoxazol-5-yl)-3- | | 22.1 Hz, 1H), 4.51-4.38 (m, 2H), |
| | | methylbutanoyl)-N-((3-(4- | | 4.31 (t, J = 5.0 Hz, 2H), 4.04 (dq, |
| | | methylthiazol-5- | | J = 14.9, 7.7 Hz, 3H), 3.87 (dd, J = |
| | | yl)bicyclo[1.1.1]pentan-1- | | 10.8, 4.2 Hz, 1H), 3.78-3.55 (m, |
| | | yl)methyl)pyrrolidine-2- | | 4H), 3.45-3.32 (m, 2H), 3.11 (t, |
| | | carboxamide | | J = 5.1 Hz, 2H), 2.37 (d, J = 11.3 Hz, |
| | | | | 4H), 2.21-2.00 (d, J = 19.1 Hz, |
| | | | | 8H), 1.05 (d, J = 6.7 Hz, 3H), 0.88 |
| | | | | (d, J = 6.7 Hz, 3H) |
| 114 | B | (2S,4R)-4-hydroxy-1-((S)-2- | 767.3 | ¹H NMR (400 MHz, Methanol-d4) δ |
| | | (3-(2-(3-(3-(2- | | 8.65 (d, J = 20.3 Hz, 1H), 8.44 (s, |
| | | hydroxyphenyl)-7H- | | 1H), 7.92 (d, J = 7.8 Hz, 1H), 7.28 |
| | | pyrrolo[2,3-c]pyridazin-6- | | (t, J = 7.5 Hz, 1H), 6.97 (t, J = 7.6 |
| | | yl)azetidin-1- | | Hz, 2H), 6.61 (s, 1H), 6.04 (s, 1H), |
| | | yl)ethoxy)isoxazol-5-yl)-3- | | 4.57-4.44 (m, 2H), 4.31 (t, J = 5.0 |
| | | methylbutanoyl)-N-((3-(4- | | Hz, 2H), 4.11 (s, 3H), 3.85-3.74 |
| | | methylthiazol-5- | | (m, 3H), 3.70 (d, J = 3.4 Hz, 2H), |
| | | yl)bicyclo[1.1.1]pentan-1- | | 3.26 (d, J = 1.7 Hz, 2H), 3.17 (d, J = |
| | | yl)methyl)pyrrolidine-2- | | 5.0 Hz, 2H), 2.38 (d, J = 14.6 Hz, |
| | | carboxamide | | 4H), 2.21 (ddd, J = 11.4, 7.9, 3.1 |
| | | | | Hz, 1H), 2.14 (s, 1H), 2.03 (s, 6H), |
| | | | | 1.01 (dd, J = 25.7, 6.6 Hz, 3H), 0.86 |
| | | | | (dd, J = 30.1, 6.7 Hz, 3H). |
| 73 | B | (2S,4R)-4-hydroxy-1-[(2R)-2- | 900.07 | ¹H NMR (400 MHz, DMSO-d6) δ |
| | | [3-{{[12-(2-hydroxyphenyl)- | | 14.16 (s, 1H), 1.23 (s, 1H), 8.98 (d, |
| | | 4,8,10,11- | | J = 1.7 Hz, 1H), 8.59 (s, 1H), 8.40 (d, |
| | | tetraazatricyclo[7.4.0.0\hat{ }{2,7} | | J = 7.7 Hz, 1H), 8.10 (s, 1H), 7.48- |
| | | trideca-1(13),2(7),9,11- | | 7.45 (m, 2H), 7.34-7.25 (m, |
| | | tetraene-4- | | 2H),7.23-7.21 (m,1H), 7.01-6.93 |
| | | carboximidoyl]carbamoyl}methoxy)- | | (m, 2H), 6.11 (s, 1H), 5.11 (s, 1H), |
| | | 1,2-oxazol-5-yl]-3- | | 5.02-4.81(m, 3H), 4.59-4.50 (m, |
| | | methylbutanoyl]-N-[(1S)-1- | | 2H), 4.37 (t, J = 7.8 Hz, 1H), 4.27 |
| | | [4-(4-methyl-1,3-thiazol-5- | | (s, 1H), 3.95 (s, 2H), 3.73-3.66 |
| | | yl)phenyl]ethyl]pyrrolidine-2- | | (m, 2H), 3.54-3.48 (m, 2H), 2.97 (s, |
| | | carboxamide | | 2H), 2.50-2.45 (m, 3H),2.40-2.38 |
| | | | | (m, 1H), 2.03 (dd, J = 22.1, 10.8 Hz, |
| | | | | 1H), 1.83-1.72 (m, 1H), 1.35 (d, |
| | | | | J = 7.0 Hz, 3H), 1.30-1.21(s, 1H), 0.94 |
| | | | | (t, J = 6.3 Hz, 3H), 0.78 (dd, J = |
| | | | | 16.0, 6.6 Hz, 3H) |
| 263 | A | (2S,4R)-4-hydroxy-1-((R)-2- | 873.3 | ¹H NMR (400 MHz, DMSO-d6) δ |
| | | (3-(4-(2-(3-(3-(2- | | 9.01-8.93 (m, 1H), 8.57 (s, 1H), |
| | | hydroxyphenyl)-7H- | | 8.33 (s, 1H), 8.28-8.21 (m, 1H), |
| | | pyrrolo[2,3-c]pyridazin-6- | | 8.06-7.99 (m, 1H), 7.52-7.36 (m, |
| | | yl)azetidin-1-yl)-2- | | 3H), 7.31 (dq, J = 6.8, 2.1 Hz, 3H), |
| | | oxoethyl)piperazin-1- | | 6.98 (d, J = 7.8 Hz, 2H), 6.67 (s, |
| | | yl)isoxazol-5-yl)-3- | | 1H), 6.16 (d, J = 2.4 Hz, 1H), 4.87 |
| | | methylbutanoyl)-N-((S)-1-(4- | | (s, 1H), 4.68 (t, J = 9.0 Hz, 1H), |
| | | (4-methylthiazol-5- | | 4.47 (dt, J = 39.8, 7.4 Hz, 2H), 4.38- |
| | | yl)phenyl)ethyl)pyrrolidine-2- | | 4.14 (m, 3H), 4.13-4.07 (m, 1H), |
| | | carboxamide | | 3.72-3.70 (m, 1H), 3.69-3.68 (m, |
| | | | | 1H), 3.46-3.42 (m, 1H), 3.31- |
| | | | | 3.26 (m, 1H), 3.23-3.13 (m, 4H), |
| | | | | 3.12-3.02 (m, 3H), 2.89 (s, 1H), |
| | | | | 2.46 (d, J = 2.7 Hz, 1H), 2.44 (d, J = |
| | | | | 2.5 Hz, 3H), 2.26 (d, J = 7.9 Hz, |
| | | | | 1H), 2.08-2.02 (m, 1H) 1.80 (dt, J = |
| | | | | 12.6, 6.3 Hz, 1H), 1.35 (d, J = 7.1 |
| | | | | Hz, 3H), 0.96 (d, J = 6.6 Hz, 3H), |
| | | | | 0.82 (d, J = 6.7 Hz, 3H) |
| 234 | A | (2S,4R)-4-hydroxy-1-[(2R)-2- | 887.40 | ¹H NMR (300 MHz, Methanol-d4) δ |
| | | {3-[4-(2-{3-[3-(2- | | 8.89 (s, 1H), 8.49 (d, J = 3.1 Hz, |
| | | hydroxyphenyl)-7H- | | 1H), 8.01-7.92 (m, 1H), 7.51- |
| | | pyrrolo[2,3-c]pyridazin-6- | | 7.36 (m, 4H), 7.36-7.25 (m, 1H), |
| | | yl]azetidin-1-yl}-2- | | 7.05-6.94 (m, 2H), 6.71 (s, 1H), |
| | | oxoacetyl)piperazin-1-yl]- | | 6.19 (s, 1H), 5.05 (d, J = 7.0 Hz, |
| | | 1,2-oxazol-5-yl}-3- | | 1H), 4.77 (d, J = 8.2 Hz, 1H), 4.62 |
| | | methylbutanoyl]-N-[(1S)-1- | | (d, J = 11.3 Hz, 1H), 4.53 (t, J = 8.0 |
| | | [4-(4-methyl-1,3-thiazol-5- | | Hz, 2H), 4.46 (s, 1H), 4.43-4.33 |
| | | yl)phenyl]ethyl]pyrrolidine-2- | | (m, 2H), 3.86 (dd, J = 10.8, 4.1 Hz, |
| | | carboxamide | | 1H), 3.75 (s, 4H), 3.68 (d, J = 9.8 |
| | | | | Hz, 2H), 3.62-3.47 (m, 1H), 3.37 |
| | | | | (d, J = 5.4 Hz, 4H), 2.50 (s, 3H), |
| | | | | 2.45-2.34 (m, 1H), 2.20 (dd, J = |
| | | | | 13.1, 8.1 Hz, 1H), 1.98 (ddd, J = |
| | | | | 13.1, 8.7, 4.5 Hz, 1H), 1.64-1.49 |
| | | | | (d, 3H), 1.07 (d, J = 6.5 Hz, 3H), |
| | | | | 0.91 (d, J = 6.8 Hz, 3H) |
| 202 | A | (2S,4R)-4-hydroxy-1-[(2S)-2- | 789.45 | ¹H NMR (300 MHz, Methanol-d4) δ |
| | | [3-(3-{3-[3-(2- | | 8.85 (d, J = 28.2 Hz, 1H), 8.41 (d, |
| | | hydroxyphenyl)-7H- | | J = 29.6 Hz, 1H), 7.99-7.83 (m, 1H), |
| | | pyrrolo[2,3-c]pyridazin-6- | | 7.53-7.24 (m, 5H), 7.04-6.92 (m, |
| | | yl]azetidin-1-yl}propyl)-1,2- | | 2H), 6.56 (d, J = 22.7 Hz, 1H), 6.31 |
| | | oxazol-5-yl]-3- | | (d, J = 16.3 Hz, 1H), 5.04-4.91 (m, |
| | | methylbutanoyl]-N-[(1S)-1- | | 1H), 4.66-4.53 (m, 1H), 4.46 (s, |
| | | [4-(4-methyl-1,3-thiazol-5- | | 1H), 4.05-3.92 (m, 1H), 3.92- |
| | | yl)phenyl]ethyl]pyrrolidine-2- | | 3.79 (m, 3H), 3.78-3.69 (m, 1H), |
| | | carboxamide | | 3.68-3.59 (m, 1H), 3.52-3.45 (m, |
| | | | | 1H), 3.45-3.37 (m, 1H), 2.73 (t, |
| | | | | J = 7.3 Hz, 2H), 2.65 (t, J = 7.6 Hz, |
| | | | | 2H), 2.53-2.34 (m, 4H), 2.31- |
| | | | | 2.17 (m, 1H), 2.04 (d, J = 13.1, 8.2, |
| | | | | 4.8 Hz, 1H), 1.89-1.73 (m, 2H), |
| | | | | 1.54 (dd, J = 38.8, 7.0 Hz, 3H), 1.03 |
| | | | | (dd, J = 33.1, 6.6 Hz, 3H), 0.88 (dd, |
| | | | | J = 20.9, 6.7 Hz, 3H). |
| 179 | A | (2S,4R)-4-hydroxy-1-[(2R)-2- | 872.5 | ¹H NMR (300 MHz, DMSO-d6) δ |
| | | {3-[1-(2-{3-[3-(2- | | 14.20 (s, 1H), 12.67 (s, 1H), 9.06 (s, |
| | | hydroxyphenyl)-7H- | | 1H), 8.65 (d, J = 2.3 Hz, 1H), 8.47 |
| | | pyrrolo[2,3-c]pyridazin-6- | | (d, J = 7.7 Hz, 1H), 8.11 (d, J = 7.9 |
| | | yl]azetidin-1- | | Hz, 1H), 7.59-7.28 (m, 5H), 7.04 |
| | | yl}acetyl)piperidin-4-yl]-1,2- | | (d, J = 7.9 Hz, 2H), 6.68 (s, 1H), |
| | | oxazol-5-yl}-3- | | 6.42 (s, 1H), 5.20-4.93 (m, 2H), |
| | | methylbutanoyl]-N-[(1S)-1- | | 4.42 (m, J = 18.0, 10.0 Hz, 3H), |
| | | [4-(4-methyl-1,3-thiazol-5- | | 4.01 (d, J = 36.2 Hz, 4H), 3.88- |
| | | yl)phenyl]ethyl]pyrrolidine-2- | | 3.68 (m, 2H), 3.68-3.49 (m, 5H), |
| | | carboxamide | | 3.22 (t, J = 12.3 Hz, 1H), 3.06 (s, |
| | | | | 1H), 2.90-2.70 (m, 1H), 2.53 (s, |
| | | | | 3H), 2.42-2.18 (m, 1H), 2.04 (d, |
| | | | | J = 30.3 Hz, 3H), 1.92-1.74 (m, 1H), |
| | | | | 1.70 (d, J = 12.2 Hz, 1H), 1.49 (m, |
| | | | | J = 22.3, 7.1 Hz, 4H), 1.04 (d, J = 6.8 |
| | | | | Hz, 3H), 0.86 (m, J = 8.7, 6.7 Hz, |
| | | | | 3H) |
| 104 | A | (2S,4R)-1-[(2R)-2-(3-{1- | 872.45 | ¹H NMR (400 MHz, DMSO-d⁶) δ |
| | | [(6R*)-6-amino-3-(2- | | 14.53-14.44 (m, 1H), 12.28- |
| | | hydroxyphenyl)- | | 12.19 (m, 1H), 8.99 (s, 1H), 8.55 (s, |
| | | 5H,7H,8H,9H-pyridazino[3,4- | | 1H), 8.41 (d, J = 7.7 Hz, 1H), 8.11 |
| | | blindole-6- | | (d, J = 8.0 Hz, 1H), 7.48-7.41 (m, |
| | | carbonyl]piperidin-4-yl}-1,2- | | 2H), 7.37 (d, J = 8.2 Hz, 2H), 7.31- |
| | | oxazol-5-yl)-3- | | 7.24 (m, 1H), 6.98-6.90 (m, 2H), |
| | | methylbutanoyl]-4-hydroxy- | | 6.36 (s, 1H), 5.10 (d, J = 3.6 Hz, |
| | | N-[(1S)-1-[4-(4-methyl-1,3- | | 1H), 4.99-4.65 (m, 3H), 4.40- |
| | | thiazol-5- | | 4.23 (m, 2H), 3.79-3.67 (m, 2H), |
| | | yl)phenyl]ethyl]pyrrolidine-2- | | 3.53-3.42 (m, 1H), 3.27-3.14 (m, |
| | | carboxamide | | 3H), 3.09-2.79 (m, 6H), 2.45 (s, |
| | | | | 3H), 2.30-2.13 (m, 2H), 2.10- |
| | | | | 1.98 (m, 2H), 1.98-1.85 (m, 2H), |
| | | | | 1.84-1.72 (m, 1H), 1.71-1.53 (m, |
| | | | | 2H), 1.38 (d, J = 7.0 Hz, 3H), 0.97 |
| | | | | (d, J = 6.6 Hz, 3H), 0.79 (d, J = 6.6 |
| | | | | Hz, 3H) |
| 103 | A | (2S,4R)-1-[(2R)-2-(3-{1- | 872.45 | ¹H NMR (400 MHz, DMSO-d⁶) δ |
| | | [(6R*)-6-amino-3-(2- | | 14.53-14.44 (m, 1H), 12.28- |
| | | hydroxyphenyl)- | | 12.19 (m, 1H), 8.99 (s, 1H), 8.55 (s, |
| | | 5H,7H,8H,9H-pyridazino[3,4- | | 1H), 8.41 (d, J = 7.7 Hz, 1H), 8.11 |
| | | blindole-6- | | (d, J = 8.0 Hz, 1H), 7.48-7.41 (m, |
| | | carbonyl]piperidin-4-yl}-1,2- | | 2H), 7.37 (d, J = 8.2 Hz, 2H), 7.31- |
| | | oxazol-5-yl)-3- | | 7.24 (m, 1H), 6.98-6.90 (m, 2H), |
| | | methylbutanoyl]-4-hydroxy- | | 6.36 (s, 1H), 5.10 (d, J = 3.6 Hz, |
| | | N-[(1S)-1-[4-(4-methyl-1,3- | | 1H), 4.99-4.65 (m, 3H), 4.40- |
| | | thiazol-5- | | 4.23 (m, 2H), 3.79-3.67 (m, 2H), |
| | | yl)phenyl]ethyl]pyrrolidine-2- | | 3.53-3.42 (m, 1H), 3.27-3.14 (m, |
| | | carboxamide | | 3H), 3.09-2.79 (m, 6H), 2.45 (s, |
| | | | | 3H), 2.30-2.13 (m, 2H), 2.10- |
| | | | | 1.98 (m, 2H), 1.98-1.85 (m, 2H), |
| | | | | 1.84-1.72 (m, 1H), 1.71-1.53 (m, |
| | | | | 2H), 1.38 (d, J = 7.0 Hz, 3H), 0.97 |
| | | | | (d, J = 6.6 Hz, 3H), 0.79 (d, J = 6.6 |
| | | | | Hz, 3H) |
| 92 | A | (2S,4R)-4-hydroxy-1-((R)-2- | 936.25 | ¹H NMR (300 MHz, Methanol-d4) δ |
| | | (3-(4-(2-(4-(3-(2- | | 8.90 (s, 1H), 8.43 (s, 1H), 8.30 (s, |
| | | hydroxyphenyl)-7H- | | 2H), 7.95 (d, J = 7.5 Hz, 1H), 7.51- |
| | | pyrrolo[2,3-c]pyridazin-5- | | 7.38 (m, 4H), 7.33-7.25 (m, 1H), |
| | | yl)piperidin-1-yl)pyrimidin-5- | | 7.04-6.94 (m, 2H), 6.48 (s, 1H), |
| | | yl)piperidin-1-yl)isoxazol-5- | | 6.16 (s, 1H), 5.10-5.01 (m, 1H), |
| | | yl)-3-methylbutanoyl)-N-((S)- | | 4.65-4.44 (m, 3H), 3.93-3.75 (m, |
| | | 1-(4-(4-methylthiazol-5- | | 3H), 3.71-3.53 (m, 2H), 3.22- |
| | | yl)phenyl)ethyl)pyrrolidine-2- | | 3.06 (m, 2H), 3.06-2.94 (m, 2H), |
| | | carboxamide | | 2.76-2.62 (m, 1H), 2.50 (s, 3H), |
| | | | | 2.45-2.35 (m, 1H), 2.26-2.14 (m, |
| | | | | 3H), 2.05-1.73 (m, 9H), 1.55 (d, |
| | | | | J = 7.0 Hz, 3H), 1.08 (d, J = 6.5 Hz, |
| | | | | 3H), 0.93 (d, J = 6.6 Hz, 3H) |
| 85 | A | (2S,4R)-4-hydroxy-1-[(2R)-2- | 825.2 | ¹H NMR (400 MHz, DMSO-d6) δ |
| | | (3-{2-[12-(2-hydroxyphenyl)- | | 12.53 (s, 1H), 8.98 (d, J = 11.4 Hz, |
| | | 4,8,10,11- | | 1H), 8.90 (d, J = 2.9 Hz, 2H), 8.81 |
| | | tetraazatricyclo[7.4.0.0δ{2,7} | | (d, J = 2.3 Hz, 1H), 8.49-8.38 (m, |
| | | ]trideca-1(13),2(7),9,11- | | 1H), 8.16 (dt, J = 8.4, 2.0 Hz, 1H), |
| | | tetraen-4-yl]pyrimidin-5-yl}- | | 7.50-7.40 (m, 2H), 7.41-7.35 (m, |
| | | 1,2-oxazol-5-yl)-3- | | 2H), 7.30 (td, J = 7.6, 1.6 Hz, 1H), |
| | | methylbutanoyl]-N-[(1S)-1- | | 7.02-6.82 (m, 3H), 5.12 (d, J = 9.3 |
| | | [4-(4-methyl-1,3-thiazol-5- | | Hz, 3H), 4.93 (p, J = 7.2 Hz, 1H), |
| | | yl)phenyl]ethyl]pyrrolidine-2- | | 4.38 (t, J = 7.9 Hz, 1H), 4.34 (s, |
| | | carboxamide | | 2H), 3.85 (d, J = 9.7 Hz, 1H), 3.76 |
| | | | | (dd, J = 10.6, 4.3 Hz, 1H), 3.51 (t, |
| | | | | J = 5.5 Hz, 1H), 3.05 (t, J = 5.9 Hz, |
| | | | | 2H), 2.45 (d, J = 9.4 Hz, 3H), 2.38- |
| | | | | 2.27 (m, 1H), 2.08-1.98 (m, 1H), |
| | | | | 1.79 (ddd, J = 12.8, 8.2, 4.7 Hz, |
| | | | | 1H), 1.49 (d, J = 7.0 Hz, 1H), 1.43- |
| | | | | 1.29 (m, 3H), 1.01 (d, J = 6.3 Hz, |
| | | | | 3H), 0.86 (t, J = 3.2 Hz, 1H), 0.83 |
| | | | | (d, J = 6.8 Hz, 3H) |
| 74 | A | 1-{5-[(2R)-1-[(2S,4R)-4- | 900.07 | ¹H NMR (400 MHz, Methanol-d₄) δ |
| | | hydroxy-2-{[(1S)-1-[4-(4- | | 8.87 (s, 1H), 8.37 (d, J = 7.7 Hz, |
| | | methyl-1,3-thiazol-5- | | 1H), 7.99-7.91 (m, 1H), 7.48- |
| | | yl)phenyl]ethyl]carbamoyl}pyrrolidin- | | 7.40 (m, 4H), 7.32-7.23 (m, 1H), |
| | | 1-yl]-3-methyl-1- | | 7.02-6.92 (m, 2H), 6.10 (s, 1H), |
| | | oxobutan-2-yl]-1,2-oxazol-3- | | 5.03 (q, J = 6.9 Hz, 1H), 4.80-4.70 |
| | | yl}-N-[12-(2-hydroxyphenyl)- | | (m, 1H), 4.51 (t, J = 8.2 Hz, 1H), |
| | | 4,8,10,11- | | 4.43 (s, 1H), 4.10 (s, 2H), 3.83 (dd, |
| | | tetraazatricyclo[7.4.0.0\hat{ }{2,7}]trideca- | | J = 10.9, 4.2 Hz, 1H), 3.78-3.49 (m, |
| | | 1(13),2(7),9,11-tetraene-4- | | 5H), 3.06-2.95 (m, 4H), 2.50-2.28 |
| | | carboximidoyl]piperidine-4- | | (m, 5H), 2.22-2.13 (m, 1H), 2.09- |
| | | carboxamide | | 1.80 (m, 5H), 1.79-1.40 (m, 3H), |
| | | | | 1.05 (dd, J = 6.5, 2.9 Hz, 3H), 0.90 |
| | | | | (dd, J = 15.6, 6.7 Hz, 3H) |
| 309 | A | (2S,4R)-4-hydroxy-N-[(1S)- | 935.40 | ¹H NMR (400 MHz, Methanol-d⁴) δ |
| | | 1-[4-(4-methyl-1,3-thiazol-5- | | 8.77 (s, 1H), 8.53 (s, 1H), 8.19 (d, |
| | | yl)phenyl]ethyl]-1-[(2S)-3- | | J = 8.5 Hz, 2H), 8.03-7.95 (m, 1H), |
| | | methyl-2-{3-[(1s,4s)-4-(2-{4- | | 7.60 (d, J = 0.8 Hz, 1H), 7.35 (s, |
| | | [3-(2-hydroxyphenyl)-7H- | | 4H), 7.31-7.26 (m, 1H), 7.01- |
| | | pyrrolo[2,3-c]pyridazin-5- | | 6.94 (m, 2H), 6.39 (s, 1H), 5.08- |
| | | yl]piperidin-1-yl}pyrimidin-5- | | 5.00 (m, 1H), 4.82-4.74 (m, 2H), |
| | | yl)cyclohexyl]-1,2-oxazol-5- | | 4.61-4.54 (m, 1H), 4.46-4.40 (m, |
| | | yl}butanoyl]pyrrolidine-2- | | 1H), 3.90 (d, J = 9.3 Hz, 1H), 3.76- |
| | | carboxamide | | 3.62 (m, 2H), 3.26-3.16 (m, 1H), |
| | | | | 3.16-3.10 (m, 1H), 3.10-2.99 (m, |
| | | | | 2H), 2.64-2.52 (m, 1H), 2.49- |
| | | | | 2.39 (m, 1H), 2.38 (s, 3H), 2.25- |
| | | | | 2.12 (m, 3H), 2.11-1.84 (m, 5H), |
| | | | | 1.78-1.57 (m, 6H), 1.46 (d, J = 7.0 |
| | | | | Hz, 3H), 1.08 (d, J = 6.7 Hz, 3H), |
| | | | | 0.91 (d, J = 6.7 Hz, 3H) |
| 68 | A | (2S,4R)-4-hydroxy-N-[(1S)- | 935.40 | ¹H NMR (400 MHz, Methanol-d⁴) δ |
| | | 1-[4-(4-methyl-1,3-thiazol-5- | | 8.86 (s, 1H), 8.59 (s, 1H), 8.19 (s, |
| | | yl)phenyl]ethyl]-1-[(2R)-3- | | 2H), 8.03-7.96 (m, 1H), 7.66 (d, |
| | | methyl-2-{3-[(1s,4s)-4-(2-{4- | | J = 0.8 Hz, 1H), 7.46-7.25 (m, 5H), |
| | | [3-(2-hydroxyphenyl)-7H- | | 7.01-6.95 (m, 2H), 6.33 (s, 1H), |
| | | pyrrolo[2,3-c]pyridazin-5- | | 5.07-4.99 (m, 1H), 4.83-4.73 (m, |
| | | yl]piperidin-1-yl}pyrimidin-5- | | 2H), 4.56-4.36 (m, 2H), 3.89- |
| | | yl)cyclohexyl]-1,2-oxazol-5- | | 3.69 (m, 2H), 3.68-3.52 (m, 2H), |
| | | yl}butanoyl]pyrrolidine-2- | | 3.18-3.10 (m, 2H), 3.10-2.97 (m, |
| | | carboxamide | | 1H), 2.65-2.55 (m, 1H), 2.47 (s, |
| | | | | 3H), 2.43-2.34 (m, 1H), 2.24- |
| | | | | 2.10 (m, 4H), 2.09-1.85 (m, 4H), |
| | | | | 1.83-1.64 (m, 6H), 1.51 (d, J = 7.0 |
| | | | | Hz, 3H), 1.07 (d, J = 6.7 Hz, 3H), |
| | | | | 0.90 (d, J = 6.7 Hz, 3H) |
| 310 | A | (2S,4R)-4-hydroxy-N-[(1S)- | 935.60 | ¹H NMR (400 MHz, Methanol-d⁴) δ |
| | | 1-[4-(4-methyl-1,3-thiazol-5- | | 8.88 (s, 1H), 8.60 (s, 1H), 8.29- |
| | | yl)phenyl]ethyl]-1-[(2S)-3- | | 8.23 (m, 2H), 8.04-7.99 (m, 1H), |
| | | methyl-2-{3-[(1r,4r)-4-(2-{4- | | 7.67 (d, J = 0.8 Hz, 1H), 7.49-7.36 |
| | | [3-(2-hydroxyphenyl)-7H- | | (m, 4H), 7.32-7.25 (m, 1H), 7.02- |
| | | pyrrolo[2,3-c]pyridazin-5- | | 6.95 (m, 2H), 6.34 (s, 1H), 5.05- |
| | | yl]piperidin-1-yl}pyrimidin-5- | | 4.96 (m, 1H), 4.59 (t, J = 8.0 Hz, |
| | | yl)cyclohexyl]-1,2-oxazol-5- | | 1H), 4.47-4.36 (m, 1H), 3.89 (d, |
| | | yl}butanoyl]pyrrolidine-2- | | J = 9.0 Hz, 1H), 3.76-3.59 (m, 2H), |
| | | carboxamide | | 3.19-3.08 (m, 2H), 2.86-2.70 (m, |
| | | | | 1H), 2.51-2.35 (m, 5H), 2.27- |
| | | | | 2.03 (m, 6H), 2.01-1.89 (m, 3H), |
| | | | | 1.84-1.69 (m, 2H), 1.69-1.53 (m, |
| | | | | 5H), 1.50 (d, J = 7.1 Hz, 3H), 1.07 |
| | | | | (d, J = 6.7 Hz, 3H), 0.90 (d, J = 6.7 |
| | | | | Hz, 3H) |
| 67 | A | (2S,4R)-4-hydroxy-N-[(1S)- | 935.60 | ¹H NMR (400 MHz, Methanol-d⁴) δ |
| | | 1-[4-(4-methyl-1,3-thiazol-5- | | 8.87 (s, 1H), 8.60 (s, 1H), 8.29- |
| | | yl)phenyl]ethyl]-1-[(2R)-3- | | 8.26 (m, 2H), 8.01 (d, J = 7.8 Hz, |
| | | methyl-2-{3-[(1r,4r)-4-(2-{4- | | 1H), 7.67 (s, 1H), 7.48-7.36 (m, |
| | | [3-(2-hydroxyphenyl)-7H- | | 4H), 7.32-7.26 (m, 1H), 7.02- |
| | | pyrrolo[2,3-c]pyridazin-5- | | 6.94 (m, 2H), 6.32 (s, 1H), 5.08- |
| | | yl]piperidin-1-yl}pyrimidin-5- | | 4.99 (m, 1H), 4.60-4.41 (m, 2H), |
| | | yl)cyclohexyl]-1,2-oxazol-5- | | 3.89-3.72 (m, 2H), 3.67-3.59 (m, |
| | | yl}butanoyl]pyrrolidine-2- | | 1H), 3.18-3.08 (m, 2H), 2.86- |
| | | carboxamide | | 2.75 (m, 1H), 2.58-2.30 (m, 5H), |
| | | | | 2.24-2.06 (m, 5H), 2.06-1.90 (m, |
| | | | | 4H), 1.83-1.69 (m, 3H), 1.69- |
| | | | | 1.56 (m, 5H), 1.53 (d, J = 7.0 Hz, |
| | | | | 3H), 1.07 (d, J = 6.7 Hz, 3H), 0.88 |
| | | | | (d, J = 6.7 Hz, 3H) |

In Table 11, the Proc. Column lists “A” for compounds prepare from an appropriate amine and carboxylic acid and “B” for compounds prepared from an appropriate amine and aldehyde (or ketone).

### Preparation of and (2S,4R)-4-hydroxy-1-[(2S)-2-[3-([3-[3-(2-hydroxyphenyl)-7H-pyrrolo[2,3-c]pyridazin-6-yl]bicyclo[1.1.1]pentan-1-yl](methyl)amino)propanamido]-3,3-dimethylbutanoyl]-N-[[4-(4-methyl-1,3-thiazol-5-yl)phenyl]methyl]pyrrolidine-2-carboxamide (Compound 293)

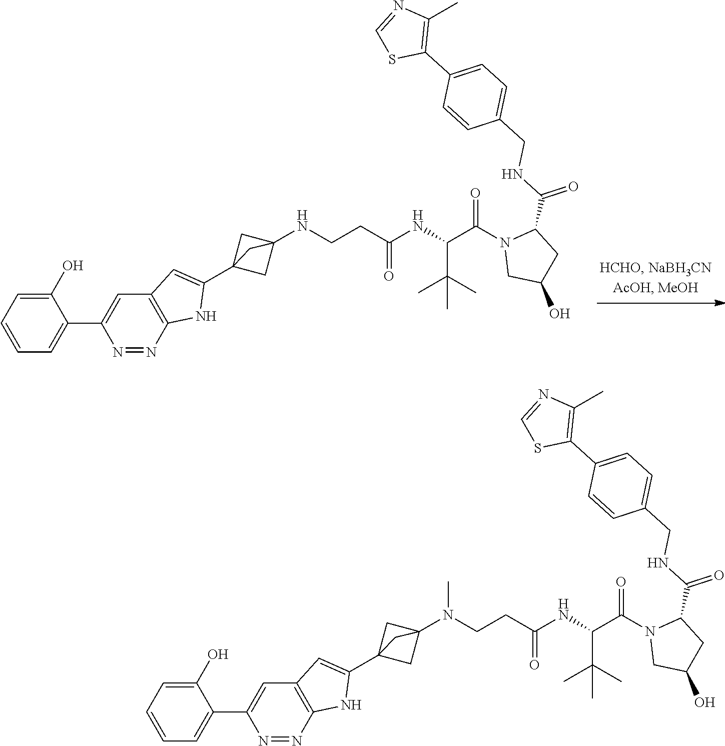 [SMILES: Cc1ncsc1-c1ccc(CNC(=O)[C@@H]2C[C@@H](O)CN2C(=O)[C@@H](NC(=O)CCNC23CC(c4cc5cc(-c6ccccc6O)nnc5[nH]4)(C2)C3)C(C)(C)C)cc1 ; Cc1ncsc1-c1ccc(CNC(=O)[C@@H]2C[C@@H](O)CN2C(=O)[C@@H](NC(=O)CCN(C)C23CC(c4cc5cc(-c6ccccc6O)nnc5[nH]4)(C2)C3)C(C)(C)C)cc1]

A solution of (2S,4R)-4-hydroxy-1-((S)-2-(3-((3-(3-(2-hydroxyphenyl)-7H-pyrrolo[2,3-c]pyridazin-6-yl)bicyclo[1.1.1]pentan-1-yl)amino)propanamido)-3,3-dimethylbutanoyl)-N-(4-(4-methylthiazol-5-yl)benzyl)pyrrolidine-2-carboxamide (40.00 mg, 0.051 mmol, 1.00 equiv), acetic acid (15.46 mg, 0.257 mmol, 5 equiv), formaldehyde (7.73 mg, 0.257 mmol, 5.00 equiv) and NaBH₃CN (16.18 mg, 0.257 mmol, 5 equiv) in DCM (1.00 mL) and MeOH (1.00 mL) was stirred for 2 h at room temperature. The reaction was monitored by LCMS. The residue was purified by Column: XBridge Shield RP18 OBD Column, 19*150 mm, 5 um; Mobile Phase A: water (10 mmol/L NH₄HCO₃), Mobile Phase B: CH₃CN; Flow rate: 25 mL/min; Gradient: 42% B to 48% B in 8 min, UV: 254/220 nm; This resulted in compound 293 (2.8 mg, 6.88%) as a white solid. ¹H NMR (400 MHz, DMSO-d6) δ 14.14 (s, 1H), 8.93 (s, 1H), 8.58 (t, J=6.0 Hz, 1H), 8.51 (s, 1H), 8.27 (d, J=9.4 Hz, 1H), 8.02 (dd, J=8.1, 1.7 Hz, 1H), 7.43 (d, J=8.3 Hz, 2H), 7.37 (d, J=8.3 Hz, 2H), 7.29 (ddd, J=8.4, 7.2, 1.5 Hz, 1H), 7.00-6.91 (m, 2H), 6.36 (s, 1H), 5.14 (s, 1H), 4.56 (d, J=9.4 Hz, 1H), 4.49-4.39 (m, 2H), 4.36 (s, 1H), 4.21 (dd, J=16.0, 5.5 Hz, 1H), 3.66 (t, J=9.4 Hz, 2H), 2.70-2.55 (m, 1H), 2.54 (s, 2H), 2.41 (s, 3H), 2.23 (s, 3H), 2.19 (s, 6H), 2.04 (s, 1H), 1.96-1.85 (m, 1H), 0.96 (s, 9H). LCMS (ESI) m/z: [M+H]⁺=791.15.

The compounds in Table 12 were prepared following protocols analogous to those above for compound 293 using the appropriate amine and formaldehyde.

TABLE 12

| | | LCMS (ESI) | |
|---|---|---|---|
| No. | Name | m/z | ¹H NMR |
| 302 | (2S,4R)-4-hydroxy-1-((R)-2- | 831.55 | 1H NMR (400 MHz, DMSO-d6) δ |
| | (3-(2-((3-(3-(2- | | 14.17 (s, 1H), 12.50 (s, 1H), 8.98 (d, J = |
| | hydroxyphenyl)-7H- | | 4.0 Hz, 1H), 8.54 (d, J = 4.4 Hz, 1H), |
| | pyrrolo[2,3-c]pyridazin-6- | | 8.43 (d, J = 7.7 Hz, 1H), 8.02 (dd, J = |
| | yl)bicyclo[1.1.1]pentan-1- | | 8.1, 1.7 Hz, 1H), 7.47-7.41 (m, 2H), |
| | yl)(methyl)amino)ethoxy)isoxazol- | | 7.40-7.33 (m, 2H), 7.29 (ddd, J = |
| | 5-yl)-3-methylbutanoyl)- | | 8.4, 7.2, 1.5 Hz, 1H), 7.00-6.90 (m, |
| | N-((S)-1-(4-(4-methylthiazol- | | 2H), 6.40 (d, J = 2.4 Hz, 1H), 6.12 (s, |
| | 5-yl)phenyl)ethyl)pyrrolidine- | | 1H), 5.13 (s, 1H), 4.91 (p, J = 7.3 Hz, |
| | 2-carboxamide | | 1H), 4.37 (t, J = 7.9 Hz, 1H), 4.31- |
| | | | 4.21 (m, 3H), 3.75-3.63 (m, 2H), |
| | | | 3.53-3.42 (m, 1H), 2.82 (t, J = 5.8 |
| | | | Hz, 2H), 2.45 (d, J = 4.9 Hz, 3H), 2.29 |
| | | | (s, 3H), 2.31-2.23 (m, 1H), 2.19 (s, |
| | | | 6H), 2.09-1.97 (m, 1H), 1.83-1.73 |
| | | | (m, 1H), 1.38 (d, J = 7.0 Hz, 3H), 0.96 |
| | | | (d, J = 6.4 Hz, 3H), 0.81 (d, J = 6.6 |
| | | | Hz, 3H). |
| 246 | (2S,4R)-4-hydroxy-1-((R)-2- | 845.50 | 1H NMR (300 MHz, Methanol-d4) δ |
| | (3-(2-(((3-(3-(2- | | 8.83 (d, J = 29.7 Hz, 1H), 8.35 (d, J = |
| | hydroxyphenyl)-7H- | | 41.6 Hz, 1H), 7.97-7.80 (m, 1H), |
| | pyrrolo[2,3-c]pyridazin-6- | | 7.49-7.39 (m, 3H), 7.32-7.22 (m, |
| | yl)bicyclo[1.1.1]pentan-1- | | 2H), 7.02-6.91 (m, 2H), 6.37 (d, J = |
| | yl)methyl)(methyl)amino)ethoxy)isoxazol- | | 22.0 Hz, 1H), 6.03 (d, J = 16.1 Hz, |
| | 5-yl)-3-methylbutanoyl)-N-((S)-1-(4- | | 1H), 5.05-5.02 (m, 1H), 4.52 (t, J = |
| | (4-methylthiazol-5- | | 8.2 Hz, 1H), 4.48-4.42 (m, 1H), 4.36 |
| | yl)phenyl)ethyl)pyrrolidine-2- | | (t, J = 5.4 Hz, 2H), 3.88-3.54 (m, |
| | carboxamide | | 6H), 2.95-2.86 (m, 2H), 2.70 (s, 2H), |
| | | | 2.46 (d, J = 16.8 Hz, 6H), 2.38 (s, 1H), |
| | | | 2.24 (s, 6H), 2.21 (s, 1H), 2.08-1.89 |
| | | | (m, 1H), 1.60-1.50 (m, 3H), 1.06 (dd, |
| | | | J = 6.6, 2.5 Hz, 3H), 0.90 (t, J = 6.9 |
| | | | Hz, 3H). |
| 242 | N-(2-((5-((R)-1-((2S,4R)-4- | 793.25 | 1H NMR (300 MHz, DMSO-d6) δ |
| | hydroxy-2-(((S)-1-(4-(4- | | 13.77 (s, 1H), 8.98 (s, 1H), 8.66 (s, |
| | methylthiazol-5- | | 1H), 8.41 (d, J = 8.0 Hz, 1H), 8.02 (d, |
| | yl)phenyl)ethyl)carbamoyl)pyrrolidin- | | J = 7.8 Hz, 1H), 7.44 (d, J = 8.0 Hz, |
| | 1-yl)-3-methyl-1-oxobutan-2- | | 2H), 7.36 (d, J = 8.0 Hz, 2H), 7.32- |
| | yl)isoxazol-3-yl)oxy)ethyl)-3-(2- | | 7.24 (m, 1H), 7.04-6.84 (m, 3H), |
| | hydroxyphenyl)-N-methyl- | | 6.15 (s, 1H), 5.17-4.99 (m, 1H), 4.97- |
| | 7H-pyrrolo[2,3-c]pyridazine- | | 4.87 (m, 1H), 4.51-4.21 (m, 3H), |
| | 6-carboxamide | | 4.08-3.86 (m, 2H), 3.68 (s, 2H), 3.43 |
| | | | (s, 1H), 3.11 (s, 2H), 2.46 (s, 3H), 2.24- |
| | | | 1.93 (m, 4H), 1.85-1.69 (m, 2H), |
| | | | 1.38 (d, J = 6.9 Hz, 3H), 0.95 (s, 3H), |
| | | | 0.87-0.67 (m, 3H). |
| 222 | (2S,4R)-4-hydroxy-1-((R)-2- | 779.50 | 1H NMR (300 MHz, DMSO-d6) δ |
| | (3-(2-(((3-(2-hydroxyphenyl)- | | 14.14 (s, 1H), 12.57 (s, 1H), 8.99 (s, |
| | 7H-pyrrolo[2,3-c]pyridazin-6- | | 1H), 8.58 (d, J = 2.2 Hz, 1H), 8.42 (d, |
| | yl)methyl)(methyl)amino)ethoxy)isoxazol- | | J = 7.7 Hz, 1H), 8.07-7.98 (m, 1H), |
| | 5-yl)-3-methylbutanoyl)- | | 7.49-7.40 (m, 2H), 7.40-7.33 (m, |
| | N-((S)-1-(4-(4-methylthiazol-5- | | 2H), 7.33-7.22 (m, 1H), 7.02-6.91 |
| | yl)phenyl)ethyl)pyrrolidine-2- | | (m, 2H), 6.56 (s, 1H), 6.10 (s, 1H), |
| | carboxamide | | 5.11 (d, J = 3.6 Hz, 1H), 4.92 (t, J = |
| | | | 7.2 Hz, 1H), 4.43-4.22 (m, 4H), 3.88 |
| | | | (s, 2H), 3.75-3.40 (m, 2H), 2.85 (t, J = |
| | | | 5.7 Hz, 2H), 2.46 (s, 3H), 2.35 (s, |
| | | | 3H), 2.33-1.91 (m, 3H), 1.88-1.67 |
| | | | (m, 1H), 1.49-1.33 (m, 3H), 0.95 (dd, |
| | | | J = 6.6, 2.8 Hz, 3H), 0.82-0.71 (m, |
| | | | 3H). |

### Preparation of (2S,4R)-4-hydroxy-1-[(2R)-2-{3-[4-(3-{3-[3-(2-hydroxyphenyl)-7H-pyrrolo[2,3-c]pyridazin-6-yl]azetidin-1-yl}-3-oxopropyl)piperazin-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 268)

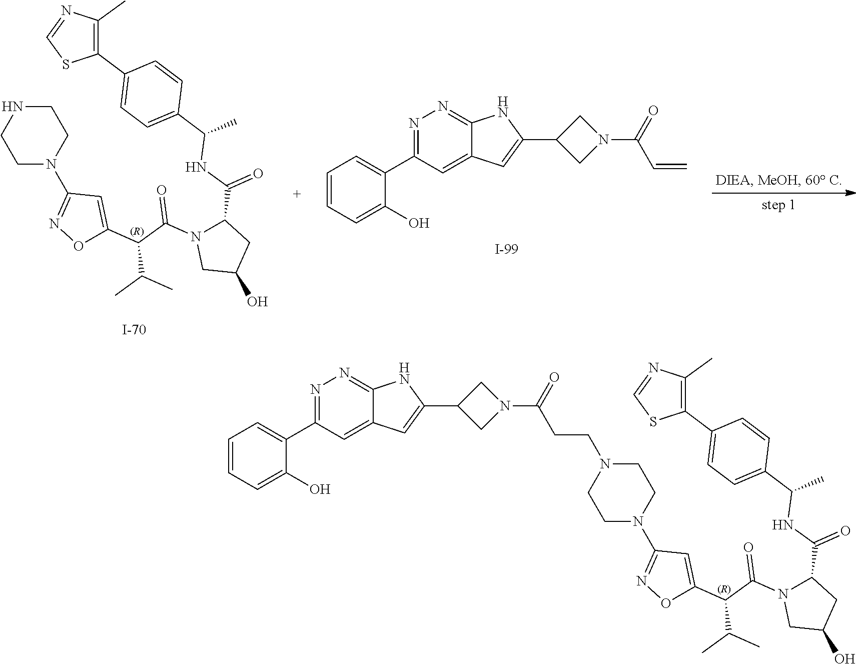

To a stirred solution of (2S,4R)-4-hydroxy-N-[(1R)-1-[4-(4-methyl-1,3-thiazol-5-yl)phenyl]ethyl]-1-[(2S)-3-methyl-2-[3-(piperazin-1-yl)-1,2-oxazol-5-yl]butanoyl]pyrrolidine-2-carboxamide (15 mg, 0.026 mmol, 1.00 equiv) and 1-{3-[3-(2-hydroxyphenyl)-7H-pyrrolo[2,3-c]pyridazin-6-yl]azetidin-1-yl}prop-2-en-1-one (8.48 mg, 0.026 mmol, 1.0 equiv) in MeOH (2 mL) was added DIEA (17.10 mg, 0.130 mmol, 5.0 equiv) dropwise at room temperature. The resulting mixture was stirred for 12 h at 60 degrees C. The resulting mixture was concentrated under reduced pressure. The residue was purified by reverse phase flash chromatography with the following conditions: column, C18 silica gel; mobile phase, MeOH in Water (0.1% TEA), 10% to 100% gradient in 25 min; detector, UV 254 nm. This resulted in compound 268 as an off-white solid. ¹H NMR (400 MHz, DMSO-d6) δ14.21 (s, 1H), 8.99 (d, J=2.1 Hz, 1H), 8.56 (s, 1H), 8.39 (d, J=7.7 Hz, 1H), 8.03 (dd, J=8.1, 1.7 Hz, 1H), 7.49-7.40 (m, 2H), 7.37 (d, J=8.1 Hz, 2H), 7.33-7.24 (m, 1H), 6.95 (t, J=8.9 Hz, 2H), 6.64 (s, 1H), 6.15 (s, 1H), 5.10 (s, 1H), 4.91 (t, J=7.2 Hz, 1H), 4.59 (t, J=8.6 Hz, 1H), 4.42-4.33 (m, 2H), 4.28 (d, J=8.5 Hz, 2H), 4.21-4.11 (m, 1H), 4.11-4.03 (m, 1H), 3.71 (dd, J=10.5, 4.4 Hz, H), 3.57 (d, J=9.9 Hz, 1H), 3.42 (d, J=10.1 Hz, 1H), 3.16 (d, J=5.4 Hz, 4H), 2.60 (d, J=7.5 Hz, 2H), 2.48-2.42 (m, 6H), 2.33-2.13 (m, 3H), 2.02 (t, J=10.2 Hz, 1H), 1.84-1.73 (m, 11H), 1.38 (d, J=7.0 Hz, 3H), 0.95 (d, J=6.6 Hz, 3H), 0.78 (d, J=6.7 Hz, 3H). LCMS (ESI) m/z: [M+H]⁺=887.30.

The compounds in Table 13 were prepared following protocols analogous to those above for compound 268 using the appropriate acrylamide and amine.

TABLE 13

| | | LCMS (ESI) | |
|---|---|---|---|
| No. | Name | m/z | ¹H NMR |
| 251 | (2S,4R)-4-hydroxy-1-((R)-2- | 887.5 | 1H NMR (300 MHz, DMSO-d6) δ |
| | (3-(4-(3-(3-(3-(2- | | 13.88 (s, 1H), 12.56 (s, 1H), 8.99 (s, |
| | hydroxyphenyl)-7H- | | 1H), 8.63 (s, 1H), 8.41 (d, J = 7.7 Hz, |
| | pyrrolo[2,3-c]pyridazin-5- | | 1H), 8.14 (s, 1H), 8.07 (dd, J = 8.4, |
| | yl)azetidin-1-yl)-3- | | 1.7 Hz, 1H), 7.51-7.41 (m, 2H), 7.37 |
| | oxopropyl)piperazin-1- | | (d, J = 8.3 Hz, 2H), 7.31 (td, J = 7.6, |
| | yl)isoxazol-5-yl)-3- | | 1.6 Hz, 1H), 7.03-6.92 (m, 2H), 6.14 |
| | methylbutanoyl)-N-((S)-1-(4- | | (s, 1H), 5.11 (s, 1H), 4.92 (t, J = 7.2 |
| | (4-methylthiazol-5- | | Hz, 1H), 4.65 (t, J = 8.3 Hz, 1H), 4.42- |
| | yl)phenyl)ethyl)pyrrolidine-2- | | 4.26 (m, 4H), 4.23-4.08 (m, 1H), |
| | carboxamide | | 4.04 (dd, J = 9.0, 6.1 Hz, 1H), 3.72 |
| | | | (dd, J = 10.6, 4.5 Hz, 1H), 3.58 (d, J = |
| | | | 9.9 Hz, 1H), 3.49-3.38 (m, 2H), 3.15 |
| | | | (t, J = 4.7 Hz, 4H), 2.61 (t, J = 7.3 Hz, |
| | | | 2H), 2.46 (s, 6H), 2.33 (t, J = 7.4 Hz, |
| | | | 2H), 2.22 (dd, J = 10.0, 6.7 Hz, 1H), |
| | | | 2.01 (d, J = 8.7 Hz, 1H), 1.79 (ddd, J = |
| | | | 12.8, 8.1, 4.8 Hz, 1H), 1.42 (d, J = |
| | | | 22.7, 7.0 Hz, 3H), 0.96 (d, J = 6.3 Hz, |
| | | | 3H), 0.81 (d, J = 11.3, 6.6 Hz, 3H). |
| 230 | (2S,4R)-4-hydroxy-1-((R)-2- | 862.5 | 1H NMR (400 MHz, DMSO-d6) δ |
| | (3-(2-((3-(3-(3-(2- | | 14.07 (s, 1H), 8.98 (s, 1H), 8.59 (d, J = |
| | hydroxyphenyl)-7H- | | 3.2 Hz, 1H), 8.41 (d, J = 7.7 Hz, 1H), |
| | pyrrolo[2,3-c]pyridazin-5- | | 8.31 (s, 1H, FA), 8.04 (dd, J = 8.0, 1.6 |
| | yl)azetidin-1-yl)-3- | | Hz, 1H), 7.47-7.41 (m, 2H), 7.40- |
| | oxopropyl)amino)ethoxy)isoxazol- | | 7.33 (m, 2H), 7.29 (ddd, J = 8.4, 7.2, |
| | 5-yl)-3-methylbutanoyl)- | | 1.6 Hz, 1H), 7.01-6.90 (m, 2H), 6.66 |
| | N-((S)-1-(4-(4-methylthiazol- | | (d, J = 2.9 Hz, 1H), 6.08 (s, 1H), 5.10 |
| | 5-yl)phenyl)ethyl)pyrrolidine- | | (s, 1H), 4.96-4.85 (m, 1H), 4.57 (t, J = |
| | 2-carboxamide | | 8.6 Hz, 1H), 4.37 (td, J = 8.0, 3.8 |
| | | | Hz, 2H), 4.29 (t, J = 8.7 Hz, 2H), 4.18 |
| | | | (t, J = 5.4 Hz, 3H), 4.10-4.04 (m, |
| | | | 1H), 3.70 (dd, J = 10.6, 4.4 Hz, 1H), |
| | | | 3.64 (d, J = 9.8 Hz, 1H), 3.47-3.41 |
| | | | (m, 2H), 2.87 (t, J = 5.5 Hz, 2H), 2.78 |
| | | | (t, J = 6.8 Hz, 2H), 2.45 (s, 3H), 2.30- |
| | | | 2.18 (m, 3H), 2.07-1.96 (m, 1H), |
| | | | 1.85-1.72 (m, 1H), 1.41 (d, J = 30.2, |
| | | | 7.0 Hz, 3H), 0.96 (d, J = 6.3 Hz, 3H), |
| | | | 0.81 (d, J = 15.1, 6.7 Hz, 3H). |

### Preparation of (R)-2-(5-methyl-6,7,8,9-tetrahydro-5H-pyrido[3′,4′:4,5]pyrrolo[2,3-c]pyridazin-3-yl)phenol (I-100) and (S)-2-(5-methyl-6,7,8,9-tetrahydro-5H-pyrido[3′,4′:4,5]pyrrolo[2,3-c]pyridazin-3-yl)phenol (I-101)

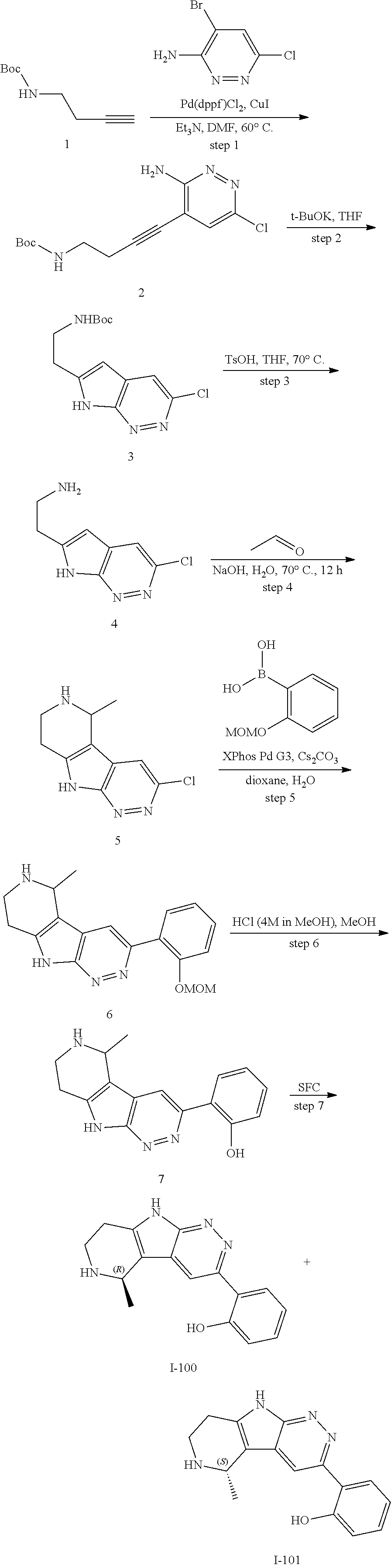

### Step 1: Preparation of tris(tert-butyl N-[4-(3-amino-6-chloropyridazin-4-yl)but-3-yn-1-yl]carbamate) (intermediate 2)

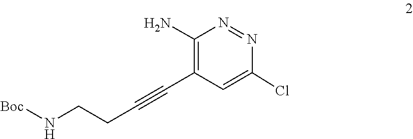 [SMILES: CC(C)(C)OC(=O)NCCC#Cc1cc(Cl)nnc1N]

To a stirred solution of tris(tert-butyl N-(but-3-yn-1-yl)carbamate) (10 g, 19.698 mmol, 1 equiv) and 4-bromo-6-chloropyridazin-3-amine (15.81 g, 75.837 mmol, 3.85 equiv) in DMF was added Pd(dppf)Cl₂ (4.76 g, 6.500 mmol, 0.33 equiv) at room temperature under an atmosphere of dry nitrogen. To the above mixture was added CuI (2.25 g, 11.814 mmol, 0.60 equiv) and Et₃N (40.00 mL, 287.788 mmol, 14.61 equiv) at room temperature under an atmosphere of dry nitrogen. The resulting mixture was stirred for additional 3 h at 60° C. Then the mixture was diluted with water (500 mL) and extracted with EtOAc (600 mL). The organic layer was washed with brine (2×250 mL), then dried over anhydrous sodium sulfate, filtered and concentrated to give crude product that was purified by chromatography on silica gel eluted with PE/EtOAc from 10/1 to 1/1 to give intermediate 2 (8.7 g, 49%) as a brown solid. LCMS (ESI) m/z: [M+H]⁺=296.9.

### Step 2: Preparation of tert-butyl N-(2-{3-chloro-7H-pyrrolo[2,3-c]pyridazin-6-yl}ethyl)carbamate. (intermediate 3)

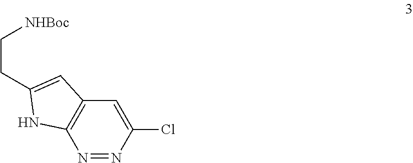 [SMILES: CC(C)(C)OC(=O)NCCc1cc2cc(Cl)nnc2[nH]1]

To a stirred solution of intermediate 2 (8.7 g, 2.247 mmol, 1 equiv) in THE was added t-BuOK (4.5 g, 40.102 mmol, 17.85 equiv). The resulting mixture was stirred for 2 h at 0-25° C. under an atmosphere of dry nitrogen. The reaction was quenched with sat. NH₄Cl (aq.) (50 mL) at 0° C., then diluted with water (300 mL×2) and extracted with EtOAc (700 mL). The organic layer was washed with brine (150 mL×2), and dried over anhydrous Na₂SO₄, and concentrated under reduced pressure to give crude product. The residue was purified by chromatography on silica gel eluted with PE/EtOAc=1/1 to give intermediate 3 (6.6 g, 75.86%) as a brown solid. LCMS (ESI) m/z: [M+H]⁺=296.9.

### Step 3: Preparation of 2-{3-chloro-7H-pyrrolo[2,3-c]pyridazin-6-yl}ethanamine (intermediate 4)

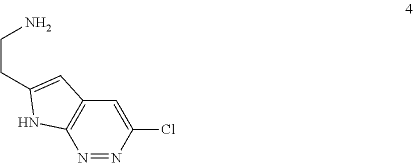 [SMILES: NCCc1cc2cc(Cl)nnc2[nH]1]

To a stirred solution of intermediate 3 (6.6 g, 22.240 mmol, 1 equiv) in THF (80 mL) was added TsOH (7659.52 mg, 44.480 mmol, 2.00 equiv) at room temperature. The resulting mixture was stirred at 70° C. for 2 h. The resulting mixture was filtered and concentrated under reduced pressure to give the intermediate 4 (5.5 g, crude) as a brown solid. LCMS (ESI) m/z: [M+H]⁺=197.1.

### Step 4: Preparation of 3-chloro-5-methyl-6,7,8,9-tetrahydro-5H-pyrido[3′,4′:4,5]pyrrolo[2,3-c]pyridazine (intermediate 5)

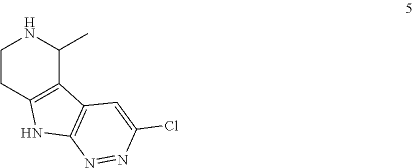

To a stirred solution of intermediate 4 (5.5 g, 27.970 mmol, 1 equiv) and acetaldehyde (2.46 g, 55.940 mmol, 2.00 equiv) in water were added H₂O (75 mL) and NaOH (1 M, 50 mL). The resulting mixture was stirred for 12 h at 70° C. The residue product was purified by reverse phase flash chromatography with the following conditions (0.04% NH₄OH) to afford intermediate 5 (2.5 g, 40.14%) as a brown solid. LCMS (ESI) m/z: [M+H]⁺=223.1.

### Step 5: Preparation of 3-(2-(methoxymethoxy)phenyl)-5-methyl-6,7,8,9-tetrahydro-5H-pyrido[3′,4′:4,5]pyrrolo[2,3-c]pyridazine (intermediate 6)

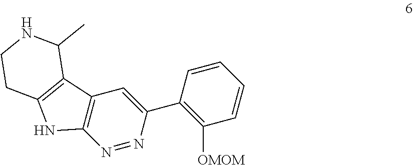

A stirred solution of intermediate 5 (2.5 g, 11.227 mmol, 1 equiv), 2-(methoxymethoxy)phenylboronic acid (4.65 g, 25.597 mmol, 2.28 equiv), XPhos Pd G3 (1.90 g, 2.245 mmol, 0.20 equiv) and Cs₂CO₃ (10.97 g, 33.681 mmol, 3 equiv) in 1,4-dioxane (35 mL) and water (7 mL) was degassed and purged with N₂ three times. The resulting mixture was stirred for 2 h at 80° C. under an atmosphere of dry nitrogen. Then the mixture was diluted with water (120 mL) and extracted with EtOAc (150 mL). The organic layer was washed with brine (2×60 mL), and dried over anhydrous sodium sulfate, filtered and concentrated to give crude product that was purified by reverse phase flash chromatography with the following conditions (0.1% FA) to afford intermediate 6 (500 mg, 13.73%) as a yellow solid. LCMS (ESI) m/z: [M+H]⁺=324.

### Step 6: Preparation of 2-(5-methyl-6,7,8,9-tetrahydro-5H-pyrido[3′,4′:4,5]pyrrolo[2,3-c]pyridazin-3-yl)phenol (intermediate 7)

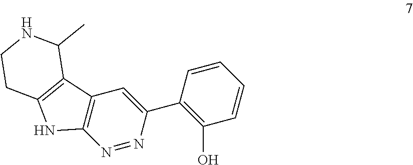 [SMILES: CC1NCCc2[nH]c3nnc(-c4ccccc4O)cc3c21]

To a stirred solution of intermediate 6 (500 mg, 1.541 mmol, 1 equiv) in MeOH (10 mL) was added HCl (4.00 mL, 4 M in MeOH). The resulting mixture was stirred for 24 h at room temperature. The mixture was concentrated to give intermediate 7 (320 mg, 74.06%) as an orange solid. LCMS (ESI) m/z: [M+H]⁺=280.

### Step 7: Preparation of (R)-2-(5-methyl-6,7,8,9-tetrahydro-5H-pyrido[3′,4′:4,5]pyrrolo[2,3-c]pyridazin-3-yl)phenol (I-100) and (S)-2-(5-methyl-6,7,8,9-tetrahydro-5H-pyrido[3′,4′:4,5]pyrrolo[2,3-c]pyridazin-3-yl)phenol (I-101)

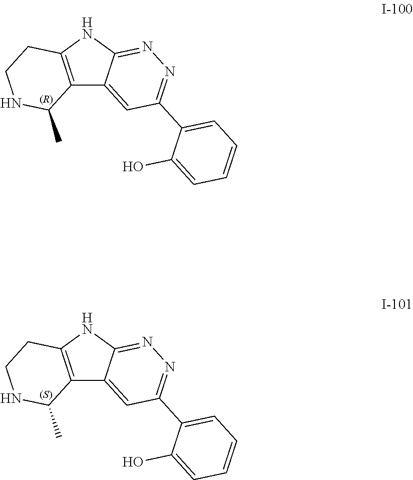

Intermediate 7 (250 mg) was purified by SFC with the following conditions: N-CHIRALPAK AD-3 (Lot No. AD3SCK-SB00113.0*100 mm, 3.0 m); Mobile Phase B: MeOH (0.1% DEA); Detector, UV 254/220 nm. This resulted in I-100 (85 mg, 19.45%) as a brown solid. ¹H NMR (400 MHz, DMSO-d6) δ 14.37 (s, 1H), 12.3 (brs, 1H), 8.47 (s, 1H), 8.19-8.10 (m, 1H), 7.28 (td, J=7.5, 1.6 Hz, 1H), 7.02-6.86 (m, 2H), 4.13 (d, J=6.6 Hz, 1H), 3.25-3.17 (m, 1H), 2.93 (ddd, J=12.5, 7.8, 4.8 Hz, 1H), 2.85-2.65 (m, 2H), 1.49 (d, J=6.6 Hz, 3H). LCMS (ESI) m/z: [M+H]⁺=280.

I-101 (58 mg, 13.27%) was obtained as a brown solid. ¹H NMR (400 MHz, DMSO-d6) δ 14.37 (s, 1H), 12.3 (brs, 1H), 8.47 (s, 1H), 8.18-8.12 (m, 1H), 7.28 (td, J=7.6, 1.6 Hz, 1H), 7.00-6.90 (m, 2H), 4.12 (d, J=6.7 Hz, 1H), 3.21 (dt, J=12.2, 4.8 Hz, 1H), 2.92 (ddd, J=12.5, 7.8, 4.7 Hz, 1H), 2.86-2.66 (m, 2H), 1.48 (d, J=6.6 Hz, 3H). LCMS (ESI) m/z: [M+H]⁺=280.

### Preparation of (2S,4R)-4-hydroxy-1-[(2R)-2-[3-(4-{[(3R)-12-(2-hydroxyphenyl)-3-methyl-4,8,10,11-tetraazatricyclo[7.4.0.0\hat{ }{2,7}]trideca-1(13),2(7),9,11-tetraen-4-yl]methyl}piperidin-1-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 81)

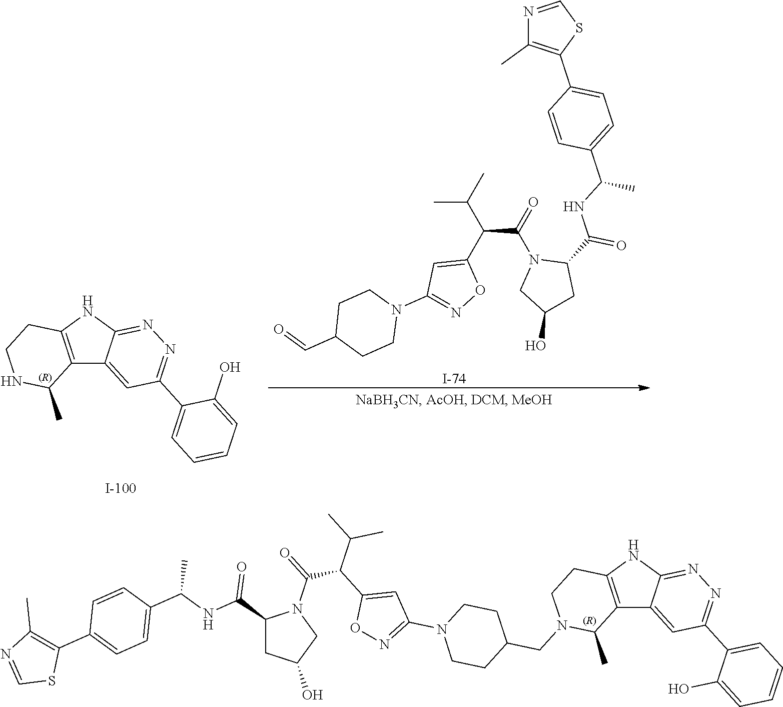

A solution of I-100 (11.80 mg, 0.042 mmol, 1 equiv) and I-74 (25 mg, 0.042 mmol, 1 equiv) in DCM (0.5 mL)/MeOH (0.5 mL)/AcOH (cat.) was stirred for 30 min at room temperature. NaBH₃CN (13.23 mg, 0.210 mmol, 5 equiv) was then added and the reaction was stirred for 2 h. The crude product was purified by Prep-HPLC (Column: XBridge Shield RP18 OBD Column, 19*150 mm, 5 μm; Mobile Phase A: Water (10 mmol/L NH₄HCO₃), Mobile Phase B: ACN; Flow rate: 25 mL/min; Gradient: 45% B to 63% B in 7 min, 63% B; Wavelength: 254/220 nm) to afford compound 81 (1.2 mg, 3.33%) as a white solid. ¹H NMR (400 MHz, Methanol-d₄) δ 8.87 (s, 1H), 8.36 (s, 1H), 8.01-7.94 (m, 1H), 7.48-7.34 (m, 4H), 7.28 (t, J=7.7 Hz, 1H), 7.01-6.93 (m, 2H), 6.10 (s, 1H), 4.58 (s, 2H), 4.51 (t, J=8.2 Hz, 1H), 4.44 (s, 1H), 4.12-4.00 (m, 1H), 3.84 (dd, J=10.8, 4.2 Hz, 1H), 3.70 (s, 3H), 3.66-3.57 (m, 3H), 2.99 (s, 1H), 2.92 (s, 1H), 2.89 (s, 3H), 2.81 (d, J=17.2 Hz, 1H), 2.61 (dd, J=12.6, 6.7 Hz, 1H), 2.55-2.45 (m, 1H), 2.48 (s, 3H), 2.35 (s, 1H), 2.17 (s, 1H), 2.01-1.85 (m, 3H), 1.58 (d, J=7.0 Hz, 1H), 1.55-1.41 (m, 8H), 1.29 (s, 6H), 1.05 (d, J=6.6 Hz, 4H), 0.90 (dd, J=11.9, 6.7 Hz, 4H). LCMS (ESI) m/z [M+H]⁺=858.4.

### Preparation of (2S,4R)-4-hydroxy-1-[(2R)-2-[3-(4-{[(3S)-12-(2-hydroxyphenyl)-3-methyl-4,8,10,11-tetraazatricyclo[7.4.0.0\hat{ }{2,7}]trideca-1(13),2(7),9,11-tetraen-4-yl]methyl}piperidin-1-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 83)

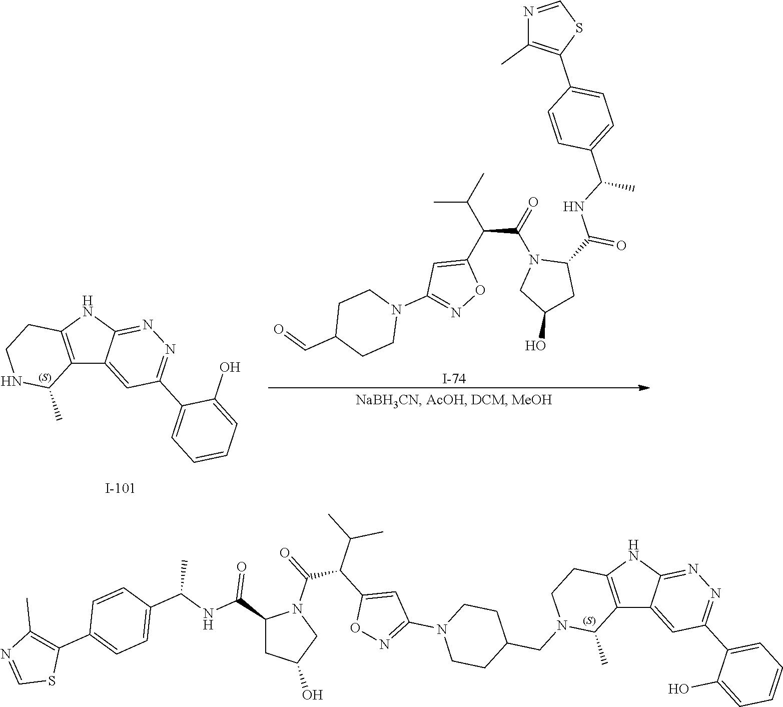

Compound 83 (1.5 mg, white solid) was prepared according to the synthetic procedure described for the preparation of compound 81 beginning from I-101 and I-74. ¹H NMR (400 MHz, Methanol-d₄) δ 8.87 (s, 1H), 8.36 (s, 1H), 7.97 (dd, J=8.3, 1.5 Hz, 1H), 7.59-7.35 (m, 4H), 7.28 (t, J=7.1 Hz, 1H), 7.06-6.92 (m, 2H), 6.07 (d, J=22.7 Hz, H), 4.51 (t, J=8.2 Hz, 1H), 4.44 (s, 1H), 4.09 (d, J=6.7 Hz, 1H), 3.84 (dd, J=10.8, 4.1 Hz, 1H), 3.70 (s, 2H), 3.66-3.48 (m, 3H), 3.12-3.05 (m, 1H), 3.01 (d, J=17.2 Hz, 1H), 2.86 (q, J=18.8, 16.4 Hz, 4H), 2.62 (dd, J=19.4, 6.7 Hz, 1H), 2.47 (d, J=2.8 Hz, 4H), 2.24-2.06 (m, 2H), 2.05-1.75 (m, 4H), 1.69-1.33 (m, 6H), 1.05 (d, J=6.6 Hz, 3H), 0.90 (dd, J=12.0, 6.7 Hz, 3H). LCMS (ESI) m/z [M+H]J=858.4.

The compounds in Table 14 were prepared using procedures similar to those used above for the preparation of compound 1 using the appropriate amine and carboxylic acid.

TABLE 14

| | | LCMS (ESI) | |
|---|---|---|---|
| No. | Name | m/z | ¹H NMR |
| 82 | (2S,4R)-4-hydroxy-1-((R)-2-(3-(4- | 872.50 | 1H NMR (400 MHz, Methanol-d4) δ 8.87 (s, |
| | ((R)-3-(2-hydroxyphenyl)-5-methyl- | | 1H), 8.48 (d, J = 26.1 Hz, 1H), 8.01 (d, J = 7.8 |
| | 6,7,8,9-tetrahydro-5H- | | Hz, 1H), 7.54-7.35 (m, 4H), 7.29 (t, J = 7.7 |
| | pyrido[3′,4′:4,5]pyrrolo[2,3- | | Hz, 1H), 6.98 (t, J = 7.1 Hz, 2H), 6.12 (d, J = |
| | c]pyridazine-6-carbonyl)piperidin-1- | | 10.6 Hz, 1H), 4.50 (dd, J = 8.1, 4.3 Hz, 2H), |
| | yl)isoxazol-5-yl)-3-methylbutanoyl)- | | 4.41 (d, J = 18.8 Hz, 2H), 3.86-3.58 (m, 6H), |
| | N-((S)-1-(4-(4-methylthiazol-5- | | 3.07-2.91 (m, 4H), 2.47 (d, J = 3.8 Hz, 3H), |
| | yl)phenyl)ethyl)pyrrolidine-2- | | 2.37 (s, 1H), 2.19 (d, J = 12.9 Hz, 1H), 2.00- |
| | carboxamide | | 1.81 (m, 4H), 1.73-1.49 (m, 7H), 1.05 (t, J = |
| | | | 5.6 Hz, 3H), 0.90 (q, J = 6.3, 5.6 Hz, 3H). |
| 86 | (2S,4R)-4-hydroxy-1-((R)-2-(3-(4- | 872.50 | 1H NMR (400 MHz, Methanol-d4) δ 8.88 (s, |
| | ((S)-3-(2-hydroxyphenyl)-5-methyl- | | 1H), 8.46 (s, 1H), 8.01 (d, J = 7.6 Hz, 1H), |
| | 6,7,8,9-tetrahydro-5H- | | 7.49-7.40 (m, 4H), 7.29 (t, J = 7.8 Hz, 1H), |
| | pyrido[3′,4′:4,5]pyrrolo[2,3- | | 6.98 (t, J = 7.2 Hz, 2H), 6.13 (s, 1H), 5.15- |
| | c]pyridazine-6-carbonyl)piperidin-1- | | 5.01 (m, 2H),4.62-4.34 (m, 2H), 3.77 (d, J = |
| | yl)isoxazol-5-yl)-3-methylbutanoyl)- | | 13.0 Hz, 2H), 3.67-3.60 (m, 3H), 3.03 (d, J = |
| | N-((S)-1-(4-(4-methylthiazol-5- | | 10.6 Hz, 4H), 2.48 (d, J = 1.3 Hz, 3H), 2.18 (s, |
| | yl)phenyl)ethyl)pyrrolidine-2- | | 1H), 1.97 (d, J = 8.8 Hz, 2H), 1.84 (d, J = 18.7 |
| | carboxamide | | Hz, 3H), 1.62-1.49 (m, 6H), 1.05 (t, J = 5.6 |
| | | | Hz, 3H), 0.89 (t, J = 5.9 Hz, 3H). |

### Preparation of (2S,4R)-4-hydroxy-1-((R)-2-(3-(1-(3-(2-hydroxyphenyl)-6,7,8,9-tetrahydro-5H-pyrido[3′,4′:4,5]pyrrolo[2,3-c]pyridazine-6-carbonyl)piperidin-4-yl)isoxazol-5-yl)-3-methylbutanoyl)-N—((S)-1-(4-(4-methylthiazol-5-yl)phenyl)ethyl)pyrrolidine-2-carboxamide (Compound 105)

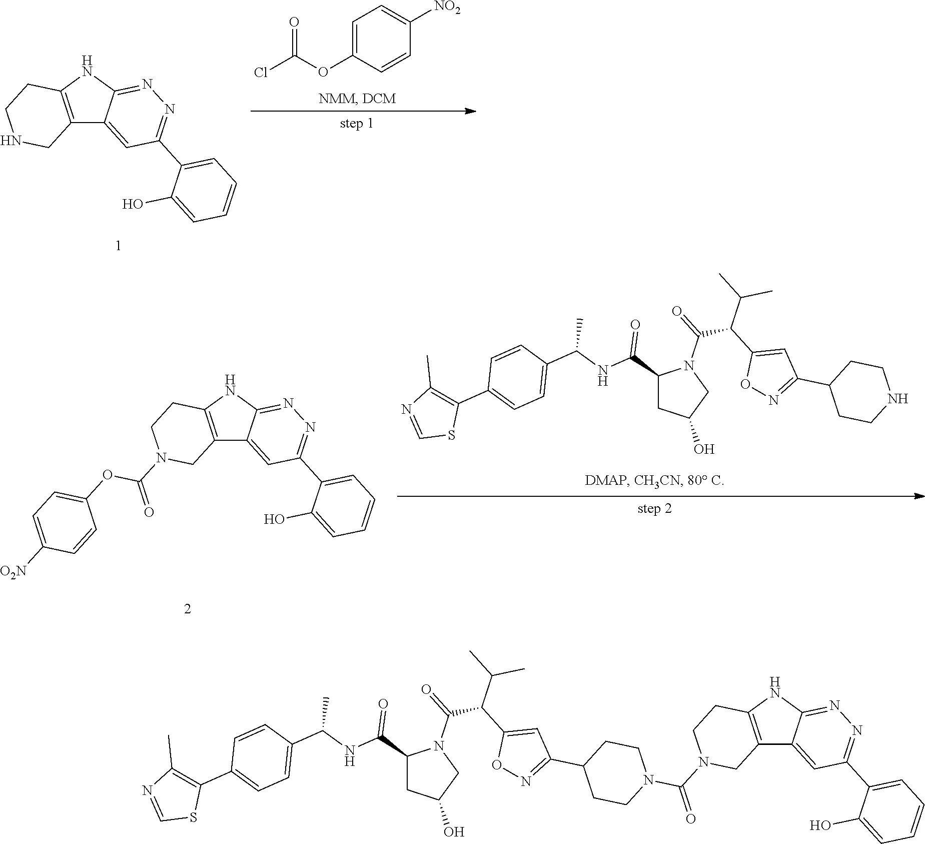 [SMILES: Oc1ccccc1-c1cc2c3c([nH]c2nn1)CCNC3 ; O=C(Cl)Oc1ccc([N+](=O)[O-])cc1 ; O=C(Oc1ccc([N+](=O)[O-])cc1)N1CCc2[nH]c3nnc(-c4ccccc4O)cc3c2C1 ; Cc1ncsc1-c1ccc([C@H](C)NC(=O)[C@@H]2C[C@@H](O)CN2C(=O)[C@@H](c2cc(C3CCNCC3)no2)C(C)C)cc1 ; Cc1ncsc1-c1ccc([C@H](C)NC(=O)[C@@H]2C[C@@H](O)CN2C(=O)[C@@H](c2cc(C3CCN(C(=O)N4CCc5[nH]c6nnc(-c7ccccc7O)cc6c5C4)CC3)no2)C(C)C)cc1]

### Step 1: Preparation of 4-nitrophenyl 3-(2-hydroxyphenyl)-5,7,8,9-tetrahydro-6H-pyrido[3′,4′:4,5]pyrrolo[2,3-c]pyridazine-6-carboxylate (Intermediate 2)

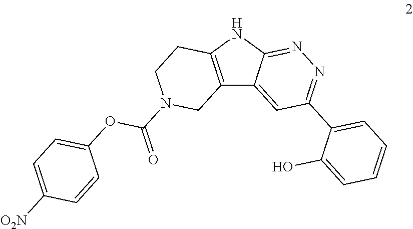 [SMILES: O=C(Oc1ccc([N+](=O)[O-])cc1)N1CCc2[nH]c3nnc(-c4ccccc4O)cc3c2C1]

To a stirred solution of 2-(6,7,8,9-tetrahydro-5H-pyrido[3′,4′:4,5]pyrrolo[2,3-c]pyridazin-3-yl)phenol (100 mg, 0.376 mmol, 1 equiv) and 4-nitrophenyl carbonochloridate (75.7 mg, 0.376 mmol, 1 equiv) in dichloromethane was added NMM (75.9 mg, 0.752 mmol, 2 equiv) dropwise at room temperature under an atmosphere of dry nitrogen. The resulting mixture was stirred for 12 h at room temperature under an atmosphere of dry nitrogen. The resulting mixture was concentrated under reduced pressure. The residue was purified by silica gel column chromatography, eluted with CH₂Cl₂/MeOH (10:1) to afford intermediate 2 (52 mg, 32%) as a yellow solid. LCMS (ESI) m/z: [M+H]⁺=432.

### Step 2: Preparation of (2S,4R)-4-hydroxy-1-((R)-2-(3-(1-(3-(2-hydroxyphenyl)-6,7,8,9-tetrahydro-5H-pyrido[3′,4′:4,5]pyrrolo[2,3-c]pyridazine-6-carbonyl)piperidin-4-yl)isoxazol-5-yl)-3-methylbutanoyl)-N—((S)-1-(4-(4-methylthiazol-5-yl)phenyl)ethyl)pyrrolidine-2-carboxamide (Compound 105)

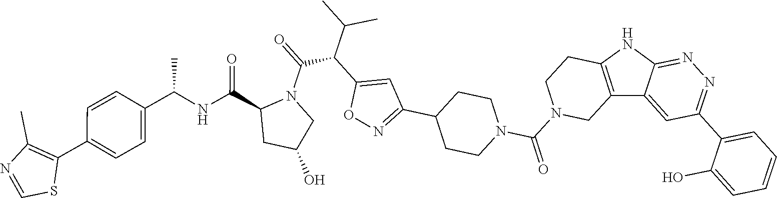 [SMILES: Cc1ncsc1-c1ccc([C@H](C)NC(=O)[C@@H]2C[C@@H](O)CN2C(=O)[C@@H](c2cc(C3CCN(C(=O)N4CCc5[nH]c6nnc(-c7ccccc7O)cc6c5C4)CC3)no2)C(C)C)cc1]

A mixture of intermediate 2 (20 mg, 0.046 mmol, 1 equiv), (2S,4R)-4-hydroxy-N-[(1S)-1-[4-(4-methyl-1,3-thiazol-5-yl)phenyl]ethyl]-1-[(2R)-3-methyl-2-[3-(piperidin-4-yl)-1,2-oxazol-5-yl]butanoyl]pyrrolidine-2-carboxamide (26.23 mg, 0.046 mmol, 1 equiv) and DMAP (cat.) in CH₃CN (0.5 mL) was stirred for 48 h at 80° C. under an atmosphere of dry nitrogen. The mixture was allowed to cool down to room temperature and then concentrated under vacuum. The crude product was purified by Prep-HPLC (Column: XBridge Shield RP18 OBD Column, 19*150 mm, 5 μm; Mobile Phase A: Water (10 mmol/L NH₄HCO₃), Mobile Phase B: ACN (0.1% FA); Flow rate: 25 mL/min; Gradient: 35% B to 56% B in 6 min; Wavelength: 254/220 nm) to afford compound 105 (4.1 mg, 10.31%) as a brown solid. ¹H NMR (400 MHz, DMSO-d6) δ 14.33 (s, 1H), 12.42 (s, 1H), 8.99 (s, 1H), 8.69 (s, 1H), 8.41 (d, J=7.6 Hz, 1H), 8.09 (d, J=8.3 Hz, 1H), 7.44 (d, J=8.2 Hz, 2H), 7.37 (d, J=8.3 Hz, 2H), 7.29 (t, J=8.3 Hz, 1H), 6.96 (d, J=8.0 Hz, 2H), 6.37 (s, 1H), 5.10 (d, J=3.5 Hz, 1H), 4.91 (p, J=7.5 Hz, 1H), 4.50 (s, 2H), 4.36 (t, J=8.0 Hz, 1H), 4.29 (s, 1H), 3.87-3.64 (m, 4H), 3.60 (t, J=4.7 Hz, 2H), 3.53-3.43 (m, 1H), 3.04-2.97 (m, 2H), 2.96-2.87 (m, 2H), 2.54 (s, 1H), 2.46 (s, 3H), 2.24 (ddd, J=16.3, 8.1, 4.4 Hz, 1H), 2.02 (t, J=10.6 Hz, 1H), 1.92 (d, J=12.6 Hz, 2H), 1.78 (ddd, J=12.6, 8.0, 4.5 Hz, 1H), 1.67 (q, J=11.7 Hz, 2H), 1.41 (dd, J=30.2, 7.0 Hz, 3H), 0.97 (d, J=6.5 Hz, 3H), 0.79 (dd, J=11.8, 6.7 Hz, 3H). LCMS (ESI) m/z: [M+H]⁺=858.2.

The compounds in Table 15 were prepared following protocols analogous to those above for compound 15 using the appropriate amine and aldehyde (or ketone) or according to the procedures analogous to those above for compound 1 using the appropriate amine and carboxylic acid.

TABLE 15

| | | | LCMS (ESI) | |
|---|---|---|---|---|
| No. | Proc. | Name | m/z | ¹H NMR |
| 183 | B | (2S,4R)-4-hydroxy-1-((R)-2-(3- | 830.30 | 1H NMR (400 MHz, Methanol-d4) δ |
| | | (4-(3-(2-hydroxyphenyl)- | | 8.89-8.80 (m, 1H), 8.39 (s, 1H), |
| | | 5,7,8,9-tetrahydro-6H- | | 7.96 (d, J = 7.9 Hz, 1H), 7.48-7.38 |
| | | pyrido[3′,4′:4,5]pyrrolo[2,3- | | (m, 3H), 7.38-7.20 (m, 2H), 7.00- |
| | | c]pyridazin-6-yl)piperidin-1- | | 6.91 (m, 2H), 6.14 (s, 1H), 5.09- |
| | | yl)isoxazol-5-yl)-3- | | 4.99 (m, 1H), 4.52 (t, J = 8.2 Hz, |
| | | methylbutanoyl)-N-((S)-1-(4- | | 1H), 4.45 (s, 1H), 3.95-3.74 (m, |
| | | (4-methylthiazol-5- | | 5H), 3.67-3.58 (m, 2H), 3.12- |
| | | yl)phenyl)ethyl)pyrrolidine-2- | | 3.06 (m, 2H), 3.04-2.84 (m, 5H), |
| | | carboxamide | | 2.48 (s, 2H), 2.42-2.34 (m, 2H), |
| | | | | 2.24-2.14 (m, 1H), 2.10-2.03 (m, |
| | | | | 2H), 2.02-1.87 (m, 1H), 1.82- |
| | | | | 1.73 (m, 2H), 1.56 (dd, J = 28.4, 7.0 |
| | | | | Hz, 3H), 1.06 (d, J = 6.5 Hz, 3H), |
| | | | | 0.95-0.85 (m, 3H). |
| 187 | A | (2S,4R)-4-hydroxy-1-((R)-2-(3- | 858.50 | 1H NMR (400 MHz, Methanol-d4) δ |
| | | (4-(3-(2-hydroxyphenyl)- | | 9.37 (d, J = 16.8 Hz, 1H), 8.60 (d, J = |
| | | 6,7,8,9-tetrahydro-5H- | | 17.8 Hz, 1H), 7.63 (d, J = 7.9 Hz, |
| | | pyrido[3′,4′:4,5]pyrrolo[2,3- | | 1H), 7.49 (t, J = 7.4 Hz, 5H), 7.16- |
| | | c]pyridazine-6- | | 7.06 (m, 2H), 6.12 (d, J = 9.5 Hz, |
| | | carbonyl)piperidin-1- | | 1H), 5.04 (d, J = 7.2 Hz, 2H), 4.91 |
| | | yl)isoxazol-5-yl)-3- | | (s, 1H), 4.53-4.44 (m, 2H), 4.14- |
| | | methylbutanoyl)-N-((S)-1-(4- | | 4.06 (m, 2H), 3.84 (d, J = 10.7 Hz, |
| | | (4-methylthiazol-5- | | 1H), 3.78 (t, J = 14.4 Hz, 2H), 3.65- |
| | | yl)phenyl)ethyl)pyrrolidine-2- | | 3.59 (m, 2H), 3.31-3.30 (m, 1H), |
| | | carboxamide | | 3.14-3.00 (m, 4H), 2.53 (s, 3H), |
| | | | | 2.39-2.33 (m, 1H), 2.23-2.13 (m, |
| | | | | 1H), 1.98-1.92 (m, 1H), 1.84-1.79 |
| | | | | (m, 4H), 1.59-1.52 (m, 3H), 1.08- |
| | | | | 1.02 (m, 3H), 0.89 (m, 3H). |
| 193 | B | (2S,4R)-4-hydroxy-1-((R)-2-(3- | 844.50 | 1H NMR (300 MHz, DMSO-d6) δ |
| | | (4-((3-(2-hydroxyphenyl)- | | 14.39 (s, 1H), 12.35 (s, 1H), 9.00 |
| | | 5,7,8,9-tetrahydro-6H- | | (d, J = 2.7 Hz, 1H), 8.56 (s, 1H), |
| | | pyrido [3′,4′:4,5]pyrrolo[2,3- | | 8.40 (d, J = 7.8 Hz, 1H), 8.06 (d, J = |
| | | c]pyridazin-6- | | 8.0 Hz, 1H), 7.51-7.41 (m, 2H), |
| | | yl)methyl)piperidin-1- | | 7.37 (d, J = 8.3 Hz, 2H), 7.29 (t, J = |
| | | yl)isoxazol-5-yl)-3- | | 7.7 Hz, 1H), 6.97 (d, J = 7.9 Hz, |
| | | methylbutanoyl)-N-((S)-1-(4- | | 2H), 6.14 (s, 1H), 5.11 (d, J = 3.6 |
| | | (4-methylthiazol-5- | | Hz, 1H), 4.92 (t, J = 7.1 Hz, 1H), |
| | | yl)phenyl)ethyl)pyrrolidine-2- | | 4.37 (t, J = 7.8 Hz, 1H), 4.29 (s, |
| | | carboxamide | | 1H), 3.68 (s, 5H), 3.57 (d, J = 9.8 |
| | | | | Hz, 1H), 3.43 (d, J = 10.6 Hz, 1H), |
| | | | | 2.96-2.69 (m, 5H), 2.46 (s, 3H), |
| | | | | 2.30-2.11 (m, 1H), 2.03 (t, J = |
| | | | | 10.2 Hz, 1H), 1.86-1.74 (m, 4H), |
| | | | | 1.38 (d, J = 7.0 Hz, 3H), 1.27-1.21 |
| | | | | (m, 2H), 0.96 (d, J = 6.4 Hz, 3H), |
| | | | | 0.90-0.70 (m, 5H). |
| 135 | B | (2S,4R)-4-hydroxy-1-((S)-2-(3- | 761.60 | 1H NMR (400 MHz, Methanol-d4) δ |
| | | ((3-(2-hydroxyphenyl)-5,7,8,9- | | 8.82 (d, J = 1.8 Hz, 1H), 8.36 (d, J = |
| | | tetrahydro-6H- | | 6.7 Hz, 1H), 7.87 (d, J = 8.1 Hz, |
| | | pyrido [3′,4′:4,5]pyrrolo[2,3- | | 1H), 7.46-7.13 (m, 5H), 6.92 (tt, J = |
| | | c]pyridazin-6- | | 15.1, 7.6 Hz, 2H), 6.50 (s, 1H), |
| | | yl)methyl)isoxazol-5-yl)-3- | | 4.78 (t, J = 7.0 Hz, 1H), 4.56 (t, J = |
| | | methylbutanoyl)-N-((S)-1-(4- | | 8.0 Hz, 1H), 4.43 (s, 1H), 3.92 (d, J = |
| | | (4-methylthiazol-5- | | 11.6 Hz, 3H), 3.86-3.79 (m, 2H), |
| | | yl)phenyl)ethyl)pyrrolidine-2- | | 3.78-3.61 (m, 2H), 3.02 (d, J = 5.9 |
| | | carboxamide | | Hz, 4H), 2.47-2.42 (d, J = 1.7 Hz, |
| | | | | 4H), 2.18 (t, J = 10.9 Hz, 1H), 2.00- |
| | | | | 1.92 (m, 1H), 1.39-1.26 (m, 3H), |
| | | | | 1.02 (dd, J = 41.7, 6.6 Hz, 3H), 0.94- |
| | | | | 0.81 (m, 3H). |
| 136 | B | (2S,4R)-4-hydroxy-1-((R)-2-(3- | 761.60 | 1H NMR (400 MHz, DMSO-d6) δ |
| | | ((3-(2-hydroxyphenyl)-5,7,8,9- | | 14.37 (d, J = 7.6 Hz, 1H), 12.37 (s, |
| | | tetrahydro-6H- | | 1H), 8.97 (d, J = 12.4 Hz, 1H), 8.56 |
| | | pyrido[3′,4′:4,5]pyrrolo[2,3- | | (s, 1H), 8.39 (d, J = 7.6 Hz, 1H), |
| | | c]pyridazin-6- | | 8.08 (dd, J = 8.2, 1.6 Hz, 1H), 7.46- |
| | | yl)methyl)isoxazol-5-yl)-3- | | 7.18 (m, 5H), 6.98-6.87 (m, 2H), |
| | | methylbutanoyl)-N-((S)-1-(4- | | 6.40 (s, 1H), 5.09 (d, J = 3.6 Hz, |
| | | (4-methylthiazol-5- | | 1H), 4.90 (p, J = 7.0 Hz, 1H), 4.35 |
| | | yl)phenyl)ethyl)pyrrolidine-2- | | (t, J = 7.9 Hz, 1H), 4.28 (s, 1H), |
| | | carboxamide | | 3.94-3.59 (m, 6H), 3.56-3.44 (m, |
| | | | | 1H), 2.97-2.92 (m, 4H), 2.45 (s, |
| | | | | 3H), 2.30-2.21 (m, 1H), 2.06- |
| | | | | 1.96 (m, 1H), 1.83-1.72 (m, 1H), |
| | | | | 1.36 (d, J = 7.0 Hz, 3H), 0.98 (d, J = |
| | | | | 6.5 Hz, 3H), 0.79 (d, J = 6.7 Hz, |
| | | | | 3H). |
| 149 | B | (2S,4R)-4-hydroxy-1-((S)-2-(3- | 804.60 | 1H NMR (400 MHz, Methanol-d4) δ |
| | | ((2-(3-(2-hydroxyphenyl)- | | 8.81 (s, 1H), 8.40-8.32 (m, 1H), |
| | | 5,7,8,9-tetrahydro-6H- | | 8.00-7.89 (m, 1H), 7.49-7.39 (m, |
| | | pyrido[3′,4′:4,5]pyrrolo[2,3- | | 1H), 7.32-7.28 (m, 2H), 7.28- |
| | | c]pyridazin-6- | | 7.22 (m, 2H), 7.00-6.90 (m, 2H), |
| | | yl)ethyl)(methyl)amino)isoxazol- | | 6.14 (s, 1H), 4.96-4.90 (m, 1H), |
| | | 5-yl)-3-methylbutanoyl)-N- | | 4.61-4.50 (m, 1H), 4.42-4.38 (m, |
| | | ((S)-1-(4-(4-methylthiazol-5- | | 1H), 3.89-3.76 (m, 2H), 3.70- |
| | | yl)phenyl)ethyl)pyrrolidine-2- | | 3.64 (m, 2H), 3.64-3.60 (m, 1H), |
| | | carboxamide | | 3.59-3.49 (m, 2H), 3.05-2.94 (m, |
| | | | | 7H), 2.93-2.77 (m, 2H), 2.50- |
| | | | | 2.38 (m, 3H), 2.38-2.28 (m, 1H), |
| | | | | 2.25-2.18 (m, 1H), 2.00-1.88 (m, |
| | | | | 1H), 1.48 (dd, J = 58.2, 7.0 Hz, 3H), |
| | | | | 1.01 (d, J = 6.6 Hz, 3H), 0.92-0.73 |
| | | | | (m, 3H). |
| 182 | B | (2S,4R)-4-hydroxy-1-((R)-2-(3- | 804.25 | 1H NMR (400 MHz, Methanol-d4) δ |
| | | ((2-(3-(2-hydroxyphenyl)- | | 8.89-8.82 (m, 1H), 8.40-8.32 (m, |
| | | 5,7,8,9-tetrahydro-6H- | | 1H), 8.00-7.86 (m, 1H), 7.47- |
| | | pyrido[3′,4′:4,5]pyrrolo[2,3- | | 7.17 (m, 5H), 7.06-6.85 (m, 2H), |
| | | c]pyridazin-6- | | 6.09 (s, 1H), 5.06-4.96 (m, 1H), |
| | | yl)ethyl)(methyl)amino)isoxazol- | | 4.49 (t, J = 8.2 Hz, 1H), 4.45-4.36 |
| | | 5-yl)-3-methylbutanoyl)-N- | | (m, 1H), 3.93-3.77 (m, 3H), 3.62- |
| | | ((S)-1-(4-(4-methylthiazol-5- | | 3.47 (m, 4H), 3.06-2.95 (m, 7H), |
| | | yl)phenyl)ethyl)pyrrolidine-2- | | 2.91-2.86 (m, 2H), 2.49-2.41 (m, |
| | | carboxamide | | 3H), 2.39-2.25 (m, 1H), 2.21- |
| | | | | 2.11 (m, 1H), 2.06-1.87 (m, 1H), |
| | | | | 1.52 (dd, J = 17.7, 7.0 Hz, 3H), 1.03- |
| | | | | 0.96 (m, 3H), 0.86-0.77 (m, 3H). |
| 303 | B | (2S,4R)-4-hydroxy-1-((R)-2-(3- | 791.45 | 1H NMR (400 MHz, DMSO-d6) δ |
| | | (2-(3-(2-hydroxyphenyl)- | | 14.39 (s, 1H), 12.18 (s, 1H), 8.98 (s, |
| | | 5,7,8,9-tetrahydro-6H- | | 1H), 8.54 (d, J = 1.6 Hz, 1H), 8.42 |
| | | pyrido[3′,4′:4,5]pyrrolo[2,3- | | (d, J = 7.7 Hz, 1H), 8.09-8.01 (m, |
| | | c]pyridazin-6- | | 1H), 7.47-7.40 (m, 2H), 7.39- |
| | | yl)ethoxy)isoxazol-5-yl)-3- | | 7.32 (m, 2H), 7.32-7.24 (m, 1H), |
| | | methylbutanoyl)-N-((S)-1-(4- | | 6.99-6.88 (m, 2H), 6.13 (s, 1H), |
| | | (4-methylthiazol-5- | | 5.10 (s, 1H), 4.90 (p, J = 7.0 Hz, |
| | | yl)phenyl)ethyl)pyrrolidine-2- | | 1H), 4.42-4.32 (m, 3H), 4.28 (s, |
| | | carboxamide | | 1H), 3.78 (s, 2H), 3.74-3.62 (m, |
| | | | | 2H), 3.60-3.41 (m, 1H), 3.03- |
| | | | | 2.91 (m, 6H), 2.45 (d, J = 3.6 Hz, |
| | | | | 3H), 2.29-2.19 (m, 1H), 2.07- |
| | | | | 1.97 (m, 1H), 1.83-1.72 (m, 1H), |
| | | | | 1.37 (d, J = 7.0 Hz, 3H), 0.95 (d, J = |
| | | | | 6.4 Hz, 3H), 0.79 (d, J = 6.7 Hz, |
| | | | | 3H). |

In Table 15, the Proc. Column lists “A” for compounds prepared from an appropriate amine and carboxylic acid and “B” for compounds prepared from an appropriate amine and aldehyde (or ketone).

### Preparation of 2-(6-{2,6-diazaspiro[3.3]heptan-2-yl}-7H-pyrrolo[2,3-c]pyridazin-3-yl)phenol (I-102)

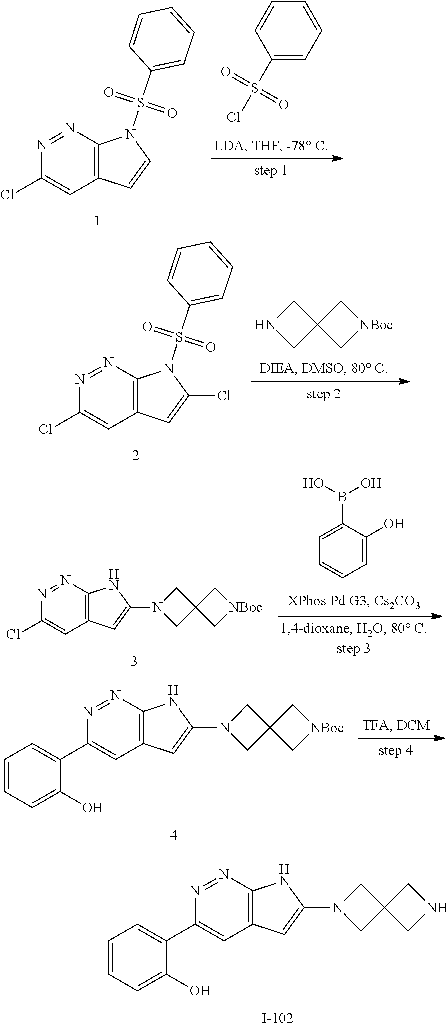

### Step 1: Preparation of 7-(benzenesulfonyl)-3,6-dichloropyrrolo[2,3-c]pyridazine (Intermediate 2)

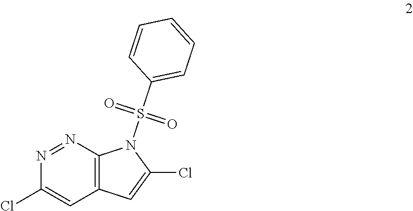 [SMILES: O=S(=O)(c1ccccc1)n1c(Cl)cc2cc(Cl)nnc21]

To a solution of 7-(benzenesulfonyl)-3-chloropyrrolo[2,3-c]pyridazine (1.5 g, 5.107 mmol, 1 equiv) in THF (30 mL) was added LDA (0.55 g, 5.107 mmol, 1.0 equiv) dropwise over 30 min at −78° C. under an atmosphere of dry nitrogen. The resulting mixture was stirred for 1 h at −78° C. Then to the above mixture was added benzenesulfonyl chloride (0.99 g, 5.618 mmol, 1.1 equiv) at −78° C. The resulting mixture was stirred for additional 1 h at −78° C. Then the reaction was quenched with sat. NH₄Cl (aq.) at −78° C. The aqueous layer was extracted with EtOAc (3×100 mL). The combined organic layers were dried over anhydrous Na₂SO₄. After filtration, the filtrate was concentrated under reduced pressure. The residue was purified by reverse phase flash chromatography with the following conditions: column, silica gel; mobile phase, MeCN in water (0.05% FA), 0% to 100% gradient in 40 min; detector, UV 254 nm. This resulted in intermediate 2 (400 mg, 23.87%) as a yellow solid. LCMS (ESI) m/z [M+H]⁺=328.

### Step 2: Preparation of tert-butyl 6-{3-chloro-7H-pyrrolo[2,3-c]pyridazin-6-yl}-2,6-diazaspiro[3.3]heptane-2-carboxylate (Intermediate 3)

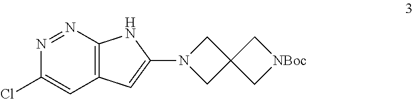 [SMILES: CC(C)(C)OC(=O)N1CC2(C1)CN(c1cc3cc(Cl)nnc3[nH]1)C2]

A solution of intermediate 2 (200 mg, 0.609 mmol, 1 equiv), DIEA (1.02 g, 7.917 mmol, 13 equiv) and tert-butyl 2,6-diazaspiro[3.3]heptane-2-carboxylate (604.16 mg, 3.045 mmol, 5 equiv) in DMSO (5 mL) was stirred for 4 h at 80° C. under an atmosphere of dry nitrogen. The residue was purified by reverse phase flash chromatography with the following conditions: column, silica gel; mobile phase, MeCN in water, 0% to 100% gradient in 50 min; detector, UV 254 nm. This resulted in intermediate 3 (70 mg, 32.83%) as a yellow solid. LCMS (ESI) m/z [M+H]⁺=350.

### Step 3: Preparation of tert-butyl 6-[3-(2-hydroxyphenyl)-7H-pyrrolo[2,3-c]pyridazin-6-yl]-2,6-diazaspiro[3.3]heptane-2-carboxylate (Intermediate 4)

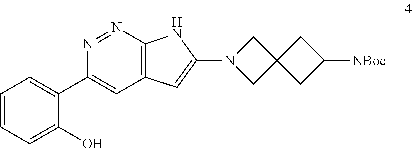

To a solution of intermediate 3 (70 mg, 0.200 mmol, 1 equiv) and 2-hydroxyphenylboronic acid (82.80 mg, 0.600 mmol, 3.0 equiv) in dioxane (2 mL) and H₂O (0.4 mL) were added Cs₂CO₃ (195.59 mg, 0.600 mmol, 3.0 equiv) and XPhos Pd G3 (33.88 mg, 0.040 mmol, 0.2 equiv). After stirring for 4 h at 80° C. under a nitrogen atmosphere, the resulting mixture was concentrated under reduced pressure. The residue was purified by Prep-TLC/silica gel column chromatography, eluted with PE/EA (1:1) to afford intermediate 4 (35 mg, 42.93%) as a yellow solid. LCMS (ESI) m/z [M+H]⁺=408.

### Step 4: Preparation of 2-(6-{2,6-diazaspiro[3.3]heptan-2-yl}-7H-pyrrolo[2,3-c]pyridazin-3-yl)phenol (I-102)

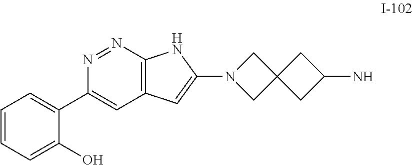 [SMILES: NC1CC2(C1)CN(c1cc3cc(-c4ccccc4O)nnc3[nH]1)C2]

A solution of intermediate 4 (30 mg, 0.074 mmol, 1 equiv) and TFA (0.5 mL) in DCM (2 mL) was stirred for 1 h at room temperature. The resulting mixture was concentrated under reduced pressure (the crude product was used in the next step directly without further purification). This resulted in I-102 (30 mg, crude) as a yellow oil. LCMS (ESI) m/z [M+H]⁺=308.

### Preparation of (2S,4R)-4-hydroxy-1-[(2R)-2-[3-(2-{6-[3-(2-hydroxyphenyl)-7H-pyrrolo[2,3-c]pyridazin-6-yl]-2,6-diazaspiro[3.3]heptan-2-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 226)

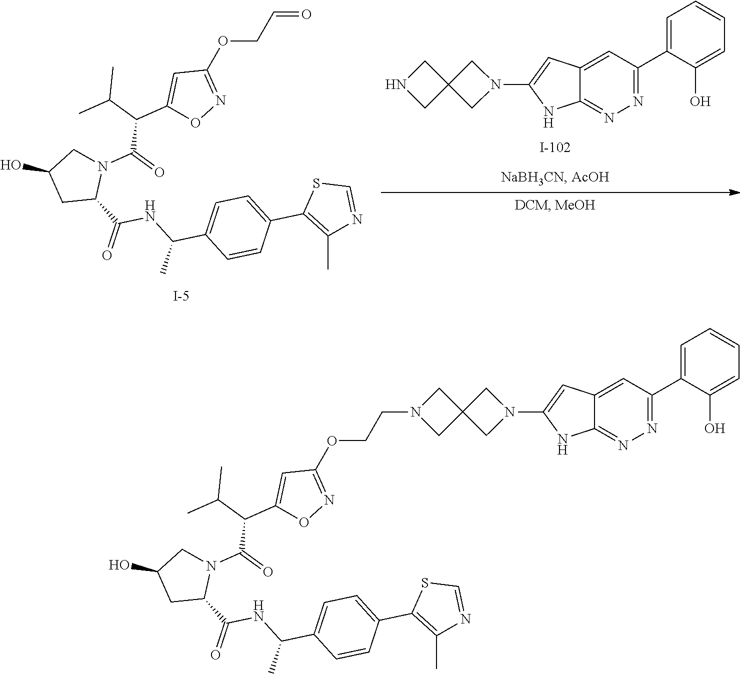

To a stirred solution of (2S,4R)-4-hydroxy-1-((R)-3-methyl-2-(3-(2-oxoethoxy)isoxazol-5-yl)butano yl)-N—((S)-1-(4-(4-methylthiazol-5-yl)phenyl)ethyl)pyrrolidine-2-carboxamide (17.59 mg, 0.033 mmol, 1.00 equiv) and I-102 (10 mg, 0.033 mmol, 1.00 equiv) in DCM (1 mL) and MeOH (1 mL) were added AcOH (0.20 mg, 0.003 mmol, 0.1 equiv) and NaBH₃CN (10.22 mg, 0.165 mmol, 5.0 equiv) at room temperature. The resulting mixture was stirred for 4 h at room temperature. The resulting mixture was concentrated under reduced pressure. The crude product was purified using the following conditions: Column, Kinetex EVO C18 Column, 21.2*150 mm, 5 m; Mobile phase, water (10 mmol/L NH₄HCO₃) and MeOH (57% MeOH up to 81% in 8 min); Detector, UV 254 nm. This resulted in compound 226 (2.9 mg, 10.71%) as an off-white solid. ¹H NMR (400 MHz, DMSO-d6) δ 15.12 (s, 1H), 12.04 (s, 1H), 8.98 (s, 1H), 8.41 (d, J=7.6 Hz, 1H), 7.99-7.90 (m, 2H), 7.49-7.33 (m, 4H), 7.28-7.19 (m, 1H), 6.89 (t, J=8.2 Hz, 2H), 6.08 (s, 1H), 5.33 (d, J=3.7 Hz, 1H), 5.11 (d, J=3.6 Hz, 1H), 4.91 (p, J=7.4 Hz, 1H), 4.37 (t, J=7.9 Hz, 1H), 4.28 (s, 1H), 4.21 (s, 4H), 4.11 (s, 2H), 3.74-3.62 (m, 2H), 3.61-3.41 (m, 4H), 2.75 (s, 1H), 2.45 (s, 3H), 2.28-2.24 (m, 1H), 2.02 (d, J=9.0 Hz, 1H), 1.78 (ddd, J=12.8, 8.1, 4.8 Hz, 1H), 1.38 (d, J=7.0 Hz, 3H), 1.00-0.93 (m, 3H), 0.87-0.77 (m, 3H). LCMS (ESI) m/z: [M+H]⁺=832.6.

The compounds in Table 16 were prepared using procedures similar to those used above for the preparation of compound 226 using the appropriate amine and aldehyde.

TABLE 16

| | | LCMS (ESI) | |
|---|---|---|---|
| No. | Name | m/z | ¹H NMR |
| 235 | (2S,4R)-4-hydroxy-1-((R)-2-(3-(2- | 820.50 | 1H NMR (300 MHz, Methanol-d4) δ 8.87 (d, J = |
| | (4-(3-(2-hydroxyphenyl)-7H- | | 15.1 Hz, 1H), 7.97-7.82 (m, 2H), 7.51- |
| | pyrrolo[2,3-c]pyridazin-6- | | 7.33 (m, 4H), 7.32-7.20 (m, 1H), 6.93 (t, J = |
| | yl)piperazin-1-yl)ethoxy)isoxazol-5- | | 8.7 Hz, 2H), 6.05 (s, 1H), 5.05 (d, J = 7.0 Hz, |
| | yl)-3-methylbutanoyl)-N-((S)-1-(4- | | 1H), 4.53 (t, J = 8.2 Hz, 1H), 4.48-4.37 (m, |
| | (4-methylthiazol-5- | | 3H), 3.86 (dd, J = 10.8, 4.2 Hz, 1H), 3.73- |
| | yl)phenyl)ethyl)pyrrolidine-2- | | 3.64 (m, 2H), 3.57 (t, J = 5.1 Hz, 4H), 2.90 (t, J = |
| | carboxamide | | 5.2 Hz, 2H), 2.77 (t, J = 5.1 Hz, 4H), 2.50 (s, |
| | | | 3H), 2.43-2.36 (m, 1H), 2.26-2.13 (m, 1H), |
| | | | 1.97 (ddd, J = 16.9, 8.5, 4.1 Hz, 1H), 1.54 (d, J = |
| | | | 7.0 Hz, 3H), 1.08 (d, J = 6.6 Hz, 3H), 0.93 |
| | | | (d, J = 6.9 Hz, 3H). |
| 245 | (2S,4R)-4-hydroxy-1-((2R)-2-(3-(2- | 846.50 | 1H NMR (400 MHz, Methanol-d4) δ 8.77 (s, |
| | (3-(3-(2-hydroxyphenyl)-7H- | | 1H), 7.77-7.68 (m, 2H), 7.37-7.21 (m, 4H), |
| | pyrrolo[2,3-c]pyridazin-6-yl)-3,8- | | 7.14 (t, J = 7.7 Hz, 1H), 6.90-6.74 (m, 2H), |
| | diazabicyclo[3.2.1 ]octan-8- | | 5.95 (s, 1H), 4.93 (d, J = 7.0 Hz, 1H), 4.40 (t, J = |
| | yl)ethoxy)isoxazol-5-yl)-3- | | 8.2 Hz, 1H), 4.36-4.25 (m, 3H), 3.74 (dd, J = |
| | methylbutanoyl)-N-((S)-1-(4-(4- | | 10.8, 4.2 Hz, 1H), 3.67-3.36 (m, 7H), 3.25 |
| | methylthiazol-5- | | (d, J = 11.0 Hz, 2H), 2.84-2.75 (m, 2H), 2.38 |
| | yl)phenyl)ethyl)pyrrolidine-2- | | (s, 3H), 2.30-2.25 (m, 1H), 2.12-1.96 (m, |
| | carboxamide | | 3H), 1.96-1.90 (m, 1H), 1.70 (d, J = 8.1 Hz, |
| | | | 2H), 1.42 (d, J = 7.0 Hz, 3H), 0.95 (d, J = 6.5 |
| | | | Hz, 3H), 0.79 (d, J = 6.9 Hz, 3H). |

### Preparation of 2-[5-(azetidin-3-ylmethoxy)-7H-pyrrolo[2,3-c]pyridazin-3-yl]phenol (I-103)

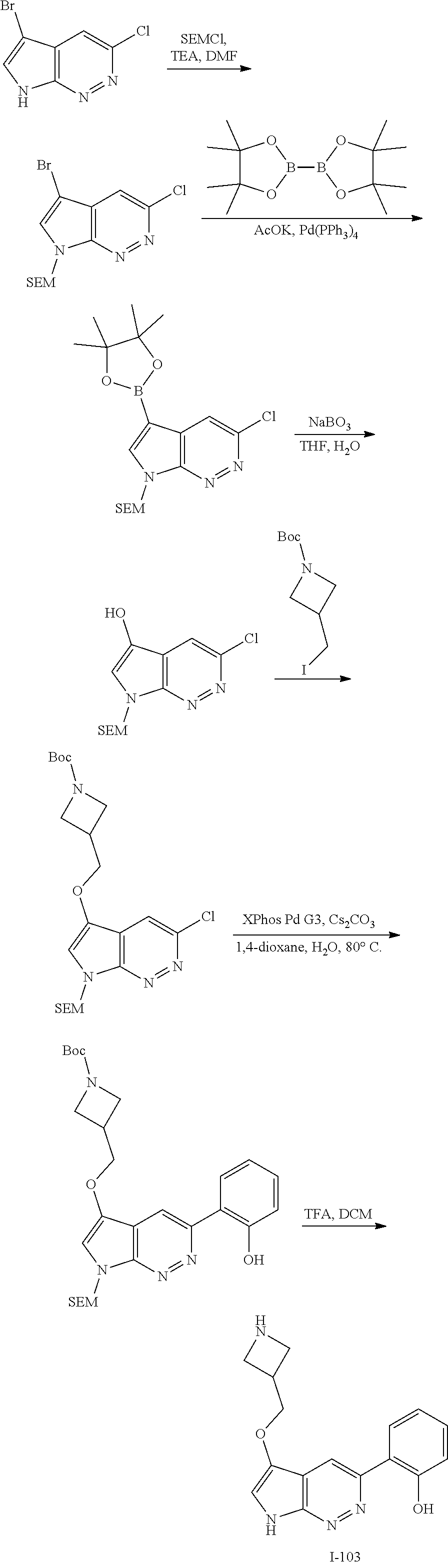

### Step 1: Preparation of 5-bromo-3-chloro-7-{[2-(trimethylsilyl)ethoxy]methyl}pyrrolo[2,3-c]pyridazine

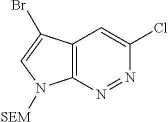 [SMILES: C[Si](C)(C)CCOCn1cc(Br)c2cc(Cl)nnc21]

To a stirred solution of 5-bromo-3-chloro-7H-pyrrolo[2,3-c]pyridazine (1000 mg, 4.302 mmol, 1.00 equiv) and SEMCI (1075.76 mg, 6.453 mmol, 1.5 equiv) in DMF (10 mL) were added TEA (870.56 mg, 8.604 mmol, 2 equiv) in portions at room temperature under an atmosphere of dry nitrogen. The resulting mixture was stirred for 1 h at room temperature under an atmosphere of dry nitrogen. The resulting mixture was extracted with EtOAc (100 mL×3). The combined organic layers were washed with brine (50 mL), and dried over anhydrous Na₂SO₄. After filtration, the filtrate was concentrated under reduced pressure. The crude product was purified by Prep-HPLC to afford 5-bromo-3-chloro-7-{[2-(trimethylsilyl)ethoxy]methyl} pyrrolo[2,3-c]pyridazine (764 mg, 48.96%) as a yellow solid. LCMS (ESI) m/z: [M+H]⁺=362.00.

### Step 2: Preparation of 3-chloro-5-(4,4,5,5-tetramethyl-1,3,2-dioxaborolan-2-yl)-7-{[2-(trimethylsilyl)ethoxy]methyl}pyrrolo[2,3-c]pyridazine

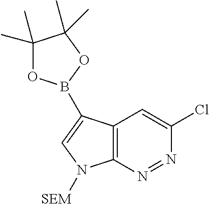 [SMILES: CC1(C)OB(c2cn(COCC[Si](C)(C)C)c3nnc(Cl)cc23)OC1(C)C]

To a stirred solution of 5-bromo-3-chloro-7-{[2-(trimethylsilyl)ethoxy]methyl} pyrrolo[2,3-c]pyridazine (630 mg, 1.737 mmol, 1.00 equiv) and bis(pinacolato)diboron (441.05 mg, 1.737 mmol, 1 equiv) in 1,4-dioxane (5 mL) were added AcOK (511.37 mg, 5.211 mmol, 3 equiv) and Pd(PPh₃)₄ (401.40 mg, 0.347 mmol, 0.2 equiv) in portions at room temperature under an atmosphere of dry nitrogen. The resulting mixture was stirred for 2 h at 100 degrees C. under an atmosphere of dry nitrogen. The resulting mixture was concentrated under reduced pressure. The crude product was used in the next step directly without further purification. LCMS (ESI) m/z: [M+H]⁺=410.18.

### Step 3: Preparation of 3-chloro-7-{[2-(trimethylsilyl)ethoxy]methyl}pyrrolo[2,3-c]pyridazin-5-ol

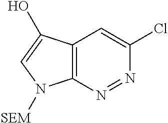 [SMILES: C[Si](C)(C)CCOCn1cc(O)c2cc(Cl)nnc21]

A stirred solution of 3-chloro-5-(4,4,5,5-tetramethyl-1,3,2-dioxaborolan-2-yl)-7-{[2-(trimethylsilyl)ethoxy]methyl}pyrrolo[2,3-c]pyridazine (630 mg, 1.537 mmol, 1.00 equiv) and oxo(sodioperoxy)borane (628.79 mg, 7.685 mmol, 5 equiv) in THF (4 mL) and H₂O (2 mL) was stirred for 30 min at room temperature. The resulting mixture was extracted with EtOAc (70 mL×3). The combined organic layers were washed with brine (50 mL), and dried over anhydrous Na₂SO₄. After filtration, the filtrate was concentrated under reduced pressure. The crude product was purified by Prep-HPLC to afford 3-chloro-7-{[2-(trimethylsilyl)ethoxy]methyl}pyrrolo[2,3-c]pyridazin-5-ol (190 mg, 39.95%) as a brown solid. LCMS (ESI) m/z: [M+H]⁺=300.09.

### Step 4: Preparation of tert-butyl 3-{[(3-chloro-7-{[2-(trimethylsilyl)ethoxy]methyl}pyrrolo[2,3-c]pyridazin-5-yl)oxy]methyl}azetidine-1-carboxylate

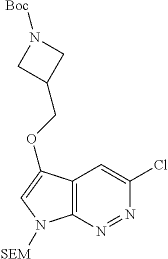 [SMILES: CC(C)(C)OC(=O)N1CC(COc2cn(COCC[Si](C)(C)C)c3nnc(Cl)cc23)C1]

To a stirred solution of 3-chloro-7-{[2-(trimethylsilyl)ethoxy]methyl}pyrrolo[2,3-c]pyridazin-5-ol (180 mg, 0.600 mmol, 1.00 equiv) and tert-butyl 3-(iodomethyl)azetidine-1-carboxylate (267.57 mg, 0.900 mmol, 1.5 equiv) in DMF (3 mL) was added Cs₂CO₃ (586.81 mg, 1.800 mmol, 3 equiv) in portions at room temperature under an atmosphere of dry nitrogen. The resulting mixture was stirred for 2 h at 100 degrees C. under an atmosphere of dry nitrogen. The resulting mixture was concentrated under reduced pressure. The crude product was purified by Prep-HPLC to afford tert-butyl 3-{[(3-chloro-7-{[2-(trimethylsilyl)ethoxy] methyl}pyrrolo[2,3-c]pyridazin-5-yl)oxy]methyl}azetidine-1-carboxylate (93 mg, 33.03%) as a yellow oil. LCMS (ESI) m/z: [M+H]⁺=489.20.

### Step 5: Preparation of tert-butyl 3-({[3-(2-hydroxyphenyl)-7-{[2-(trimethylsilyl)ethoxy]methyl}pyrrolo[2,3-c]pyridazin-5-yl]oxy}methyl)azetidine-1-carboxylate

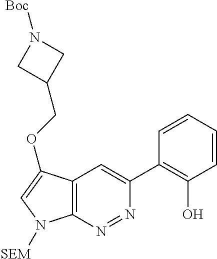 [SMILES: CC(C)(C)OC(=O)N1CC(COc2cn(COCC[Si](C)(C)C)c3nnc(-c4ccccc4O)cc23)C1]

To a stirred solution of tert-butyl 3-{[(3-chloro-7-{[2-(trimethylsilyl)ethoxy] methyl}pyrrolo[2,3-c]pyridazin-5-yl)oxy]methyl}azetidine-1-carboxylate (82 mg, 0.175 mmol, 1.00 equiv) and 2-hydroxyphenylboronic acid (72.34 mg, 0.525 mmol, 3 equiv) in 1,4-dioxane (2 mL) and H₂O (0.4 mL) were added XPhos Pd G3 (29.60 mg, 0.035 mmol, 0.2 equiv) and Cs₂CO₃ (170.88 mg, 0.525 mmol, 3 equiv) at room temperature under an atmosphere of dry nitrogen. The resulting mixture was stirred for 2 h at 85 degrees C. under an atmosphere of dry nitrogen. The resulting mixture was extracted with EtOAc (30 mL×3). The combined organic layers were washed with brine (20 mL), and dried over anhydrous Na₂SO₄. After filtration, the filtrate was concentrated under reduced pressure. The residue was purified by silica gel column chromatography, eluted with PE/EA (1:1) to afford tert-butyl 3-({[3-(2-hydroxyphenyl)-7-{[2-(trimethylsilyl)ethoxy]methyl}pyrrolo[2,3-c]pyridazin-5-yl]oxy}methyl)azetidine-1-carboxylate (57.2 mg, 62.12%) as a yellow oil. LCMS (ESI) m/z: [M+H]⁺=527.26.

### Step 6: Preparation of 2-[5-(azetidin-3-ylmethoxy)-7H-pyrrolo[2,3-c]pyridazin-3-yl]phenol (I-103)

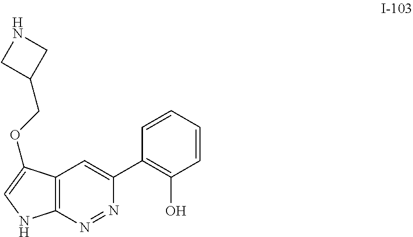 [SMILES: Oc1ccccc1-c1cc2c(OCC3CNC3)c[nH]c2nn1]

To a stirred solution of tert-butyl 3-({[3-(2-hydroxyphenyl)-7-{[2-(trimethylsilyl)ethoxy]methyl}pyrrolo[2,3-c]pyridazin-5-yl]oxy}methyl)azetidine-1-carboxylate (60 mg, 0.114 mmol, 1.00 equiv) in DCM (2.5 mL) was added TFA (0.83 mL) in portions at room temperature. The resulting mixture was stirred for 1 h at room temperature. The resulting mixture was concentrated under reduced pressure. The crude product (60 mg) was purified by Prep-HPLC with the following conditions (Column: SunFire Prep C18 OBD Column, 19*150 mm, 5 μm 10 nm; Mobile Phase A: Water (0.05% FA), Mobile Phase B: ACN; Flow rate: 25 mL/min; Gradient: 5% B to 19% B in 4 min; Wave Length: 254/220 nm;) to afford I-103 (6.6 mg, 16.92%) as a yellow solid. ¹H NMR (400 MHz, DMSO-d6) δ 13.72 (s, 1H), 12.19 (d, J=2.7 Hz, 1H), 8.66 (s, 2H), 8.06 (dd, J=8.0, 1.6 Hz, 1H), 7.76 (d, J=2.6 Hz, 1H), 7.40-7.26 (m, 1H), 7.08-6.90 (m, 2H), 4.25 (d, J=5.2 Hz, 2H), 4.14 (p, J=9.1, 8.3 Hz, 2H), 3.97 (dq, J=12.1, 6.5 Hz, 2H), 3.28-3.23 (m, 1H). LCMS (ESI) m/z: [M+H]⁺=297.13.

### Preparation of 2-[5-(azetidin-3-yloxy)-7H-pyrrolo[2,3-c]pyridazin-3-yl]phenol (I-104)

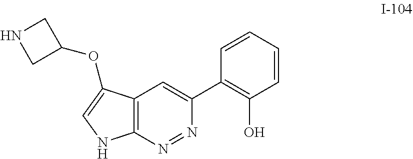 [SMILES: Oc1ccccc1-c1cc2c(OC3CNC3)c[nH]c2nn1]

Compound I-104 was obtained as a yellow solid following a similar protocol as above 1-103. ¹H NMR (300 MHz, DMSO-d6) δ 13.64 (br s, 1H), 8.59 (s, 1H), 8.28 (s, 1H, FA), 8.12 (dd, J=8.0, 1.6 Hz, 1H), 7.63 (s, 1H), 7.30 (ddd, J=8.4, 7.1, 1.6 Hz, 1H), 7.05-6.89 (m, 2H), 5.06 (s, 1H), 4.07 (s, 2H), 3.83 (s, 2H). LCMS (ESI) m/z: [M+H]⁺=283.10.

### Preparation of 2-[6-(azetidin-3-yl)-5-cyclopropyl-7H-pyrrolo[2,3-c]pyridazin-3-yl]phenol (I-105)

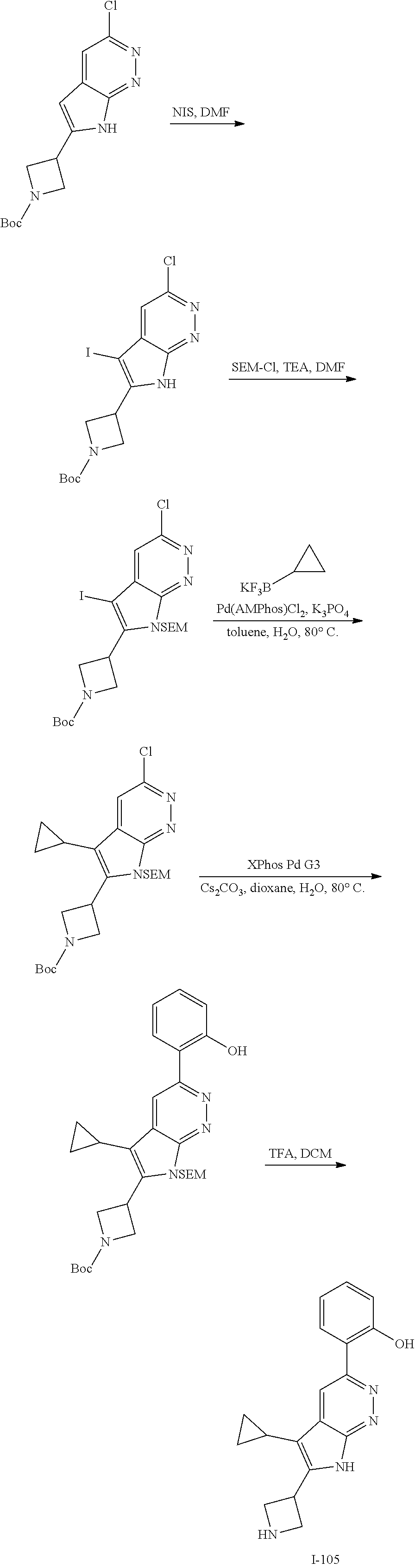

### Step 1: Preparation of tert-butyl 3-{3-chloro-5-iodo-7H-pyrrolo[2,3-c]pyridazin-6-yl}azetidine-1-carboxylate

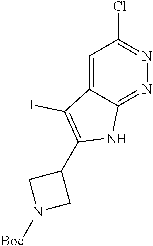 [SMILES: CC(C)(C)OC(=O)N1CC(c2[nH]c3nnc(Cl)cc3c2I)C1]

A solution of tert-butyl 3-{3-chloro-7H-pyrrolo[2,3-c]pyridazin-6-yl}azetidine-1-carboxylate (800 mg, 2.591 mmol, 1.00 equiv) and NIS (582.92 mg, 2.591 mmol, 1 equiv) in DMF (8.00 mL) was stirred for 1 h at 0° C. under an atmosphere of dry nitrogen. The residue was purified by reverse phase flash with the following conditions (Mobile Phase A: Water (0.1% FA), Mobile Phase B: CH₃CN; Flow rate: 60 mL/min; Gradient: 0% B to 100% B in 40 min; 254/220 nm) to afford tert-butyl 3-{3-chloro-5-iodo-7H-pyrrolo[2,3-c]pyridazin-6-yl}azetidine-1-carboxylate (840 mg, 70.86%) as a brown solid. LCMS (ESI) m/z: [M+H]⁺=435.

### Step 2: Preparation of tert-butyl 3-(3-chloro-5-iodo-7-{[2-(trimethylsilyl)ethoxy]methyl}pyrrolo [2,3-c]pyridazin-6-yl)azetidine-1-carboxylate

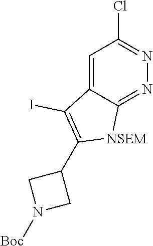 [SMILES: CC(C)(C)OC(=O)N1CC(c2c(I)c3cc(Cl)nnc3n2COCC[Si](C)(C)C)C1]

To a stirred solution of tert-butyl 3-{3-chloro-5-iodo-7H-pyrrolo[2,3-c]pyridazin-6-yl}azetidine-1-carboxylate (840 mg, 1.933 mmol, 1.00 equiv) and TEA (782.22 mg, 7.732 mmol, 4 equiv) in DMF (8.00 mL) was added SEM-CI (644.39 mg, 3.866 mmol, 2 equiv) dropwise at 0° C. under an atmosphere of dry nitrogen. The resulting mixture was stirred for 3 h at 0° C. The reaction was quenched with water/ice at 0° C. The resulting mixture was extracted with EtOAc (3×10 mL). The combined organic layers were washed with brine (2×10 mL), and dried over anhydrous Na₂SO₄. After filtration, the filtrate was concentrated under reduced pressure. The crude product was purified by Prep-TLC (PE/EA 2:1) to afford tert-butyl 3-(3-chloro-5-iodo-7-{[2-(trimethylsilyl)ethoxy]methyl}pyrrolo[2,3-c]pyridazin-6-yl)azetidine-1-carboxylate (1 g, 87.02%) as an orange solid. LCMS (ESI) m/z: [M+H]⁺=565.

### Step 3: Preparation of tert-butyl 3-(3-chloro-5-cyclopropyl-7-{[2-(trimethylsilyl)ethoxy]methyl}pyrrolo[2,3-c]pyridazin-6-yl)azetidine-1-carboxylate

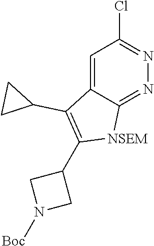 [SMILES: CC(C)(C)OC(=O)N1CC(c2c(C3CC3)c3cc(Cl)nnc3n2COCC[Si](C)(C)C)C1]

To a stirred solution of tert-butyl 3-(3-chloro-5-iodo-7-{[2-(trimethylsilyl)ethoxy]methyl}pyrrolo[2,3-c]pyridazin-6-yl)azetidine-1-carboxylate (1 g, 1.770 mmol, 1.00 equiv) and cyclopropyltrifluoro-lambda4-borane (1.93 g, 17.700 mmol, 10 equiv) in dioxane (10.00 mL) and H₂O (2.00 mL) were added Pd(dppf)Cl₂·CH₂Cl₂ (0.14 g, 0.177 mmol, 0.1 equiv) and K₂CO₃ (0.73 g, 5.310 mmol, 3 equiv). The resulting mixture was stirred overnight at 80 degrees C. under an atmosphere of dry nitrogen. The resulting mixture was added H₂O (20 mL), then the mixture was extracted with EtOAc (3×20 mL). The combined organic layers were washed with brine (30 mL), and dried over anhydrous Na₂SO₄. After filtration, the filtrate was concentrated under reduced pressure. The crude product was purified by Prep-TLC (PE/EA 1:1) to afford tert-butyl 3-(3-chloro-5-cyclopropyl-7-{[2-(trimethylsilyl)ethoxy]methyl}pyrrolo[2,3-c]pyridazin-6-yl)azetidine-1-carboxylate (295 mg, 30.55%) as an orange oil. LCMS (ESI) m/z: [M+H]⁺=479.

### Step 4: Preparation of tert-butyl 3-[5-cyclopropyl-3-(2-hydroxyphenyl)-7-{[2-(trimethylsilyl) ethoxy]methyl}pyrrolo[2,3-c]pyridazin-6-yl]azetidine-1-carboxylate

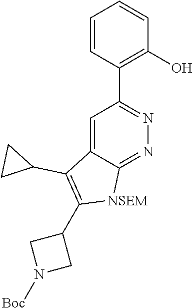 [SMILES: CC(C)(C)OC(=O)N1CC(c2c(C3CC3)c3cc(-c4ccccc4O)nnc3n2COCC[Si](C)(C)C)C1]

To a stirred solution of tert-butyl 3-(3-chloro-5-cyclopropyl-7-{[2-(trimethylsilyl)ethoxy]methyl}pyrrolo[2,3-c]pyridazin-6-yl)azetidine-1-carboxylate (167 mg, 0.349 mmol, 1.00 equiv) and 2-hydroxyphenylboronic acid (144.24 mg, 1.047 mmol, 3 equiv) in dioxane (4.00 mL) and H₂O (0.80 mL) were added XPhos Pd G3 (29.51 mg, 0.035 mmol, 0.1 equiv) and Cs₂CO₃ (340.72 mg, 1.047 mmol, 3 equiv) in portions at room temperature under an atmosphere of dry nitrogen. The resulting mixture was stirred for 1 h at 100 degrees C. under an atmosphere of dry nitrogen. The residue was filtered. The filtrate was concentrated under reduced pressure and purified by reverse phase flash with the following conditions (Mobile Phase A: Water (0.1% FA), Mobile Phase B: ACN; Flow rate: 35 mL/min; Gradient: 0% B to 100% B in 40 min; 254/220 nm) to afford tert-butyl 3-[5-cyclopropyl-3-(2-hydroxyphenyl)-7-{[2-(trimethylsilyl)ethoxy]methyl}pyrrolo[2,3-c]pyridazin-6-yl]azetidine-1-carboxylate (160 mg, 81.24%) as a yellow solid. LCMS (ESI) m/z: [M+H]⁺=537.

### Step 5: Preparation of 2-[6-(azetidin-3-yl)-5-cyclopropyl-7H-pyrrolo[2,3-c]pyridazin-3-yl]phenol (I-105)

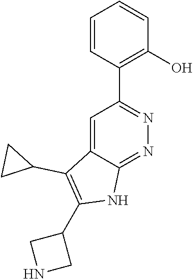 [SMILES: Oc1ccccc1-c1cc2c(C3CC3)c(C3CNC3)[nH]c2nn1]

A solution of tert-butyl 3-[5-cyclopropyl-3-(2-hydroxyphenyl)-7-{[2-(trimethylsilyl)ethoxy]methyl}pyrrolo[2,3-c]pyridazin-6-yl]azetidine-1-carboxylate (144 mg, 0.268 mmol, 1.00 equiv) in TFA (2.00 mL) was stirred for 2 h at 90° C. The resulting mixture was concentrated under reduced pressure. The crude product was purified by reverse phase flash with the following conditions (Mobile Phase A: Water (0.1% FA), Mobile Phase B: ACN; Flow rate: 35 mL/min; Gradient: 0% B to 50% B in 30 min; 254/220 nm) to afford 2-[6-(azetidin-3-yl)-5-cyclopropyl-7H-pyrrolo[2,3-c]pyridazin-3-yl]phenol (50 mg, 57.79%) as a yellow solid. LCMS (ESI) m/z: [M+H]⁺=307.

### Preparation of 2-(5,6,7,8-tetrahydropyrrolo[3′,4′:4,5]pyrrolo[2,3-c]pyridazin-3-yl)phenol (I-106)

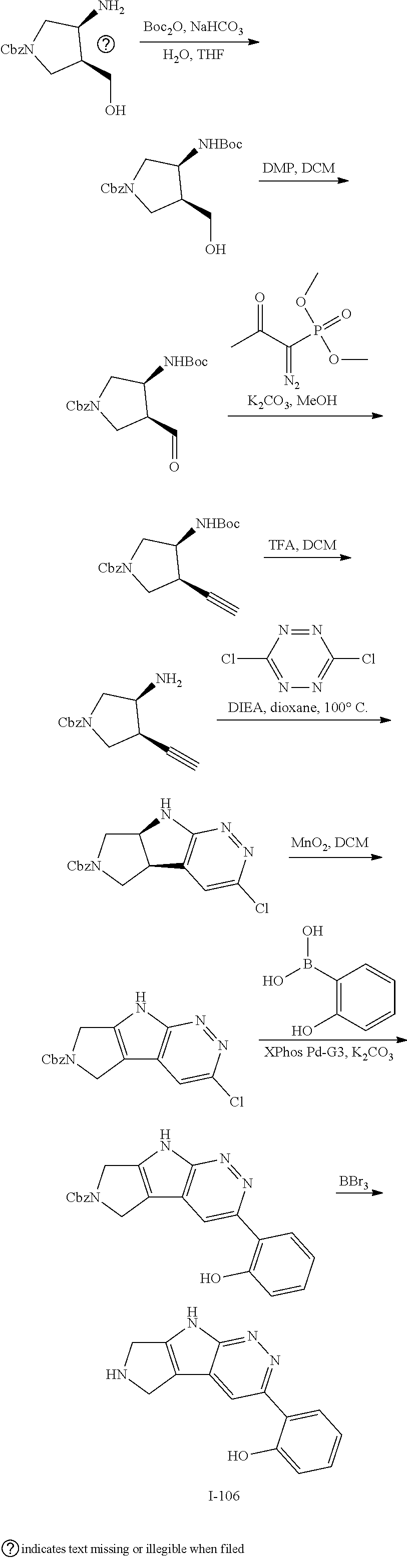

### Step 1: Preparation of benzyl (3S,4S)-3-((tert-butoxycarbonyl)amino)-4-(hydroxymethyl)pyrrolidine-1-carboxylate

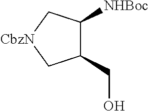 [SMILES: CC(C)(C)OC(=O)N[C@@H]1CN(C(=O)OCc2ccccc2)C[C@@H]1CO]

To a mixture of benzyl (3S, 4S)-3-amino-4-(hydroxymethyl)pyrrolidine-1-carboxylate (2.00 g, 7.990 mmol, 1.00 equiv) and Boc₂O (2.62 g, 11.985 mmol, 1.50 equiv) in THF (30 mL) and H₂O (10 mL) was added NaHCO₃ (2.01 g, 23.970 mmol, 3.00 equiv). The resulting mixture was stirred for 3 h at room temperature. The resulting mixture was diluted with water (50 mL), and the resulting mixture was extracted with EtOAc (3×100 mL). The combined organic layers were washed with brine (50 mL), and dried over anhydrous sodium sulfate. After filtration, the filtrate was concentrated under reduced pressure to afford benzyl (3S,4S)-3-((tert-butoxycarbonyl)amino)-4-(hydroxymethyl)pyrrolidine-1-carboxylate (3.50 g, crude) as a white solid which was used in the next step directly without further purification. LCMS (ESI) m/z: [M+H]⁺=351.

### Step 2: Preparation of benzyl (3S,4S)-3-((tert-butoxycarbonyl)amino)-4-formylpyrrolidine-1-carboxylate

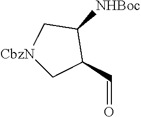 [SMILES: CC(C)(C)OC(=O)N[C@@H]1CN(C(=O)OCc2ccccc2)C[C@@H]1C=O]

To a mixture of benzyl (3S,4S)-3-((tert-butoxycarbonyl)amino)-4-(hydroxymethyl)pyrrolidine-1-carboxylate (3.50 g, 9.988 mmol, 1.00 equiv) in DCM (60 mL) was added DMP (6.35 g, 14.982 mmol, 1.50 equiv). The resulting mixture was stirred for 2 hrs at room temperature. The resulting mixture was quenched with saturated sodium thiosulfate solution (50 mL) and saturated sodium bicarbonate solution (50 mL), the resulting mixture was extracted with EtOAc (3×200 mL). The combined organic layers were washed with brine (50 mL), and dried over anhydrous sodium sulfate. After filtration, the filtrate was concentrated under reduced pressure. The residue was purified by silica gel column chromatography, eluted with EtOAc in PE from 0% to 100% to afford benzyl (3S,4S)-3-((tert-butoxycarbonyl)amino)-4-formylpyrrolidine-1-carboxylate (1.10 g, 28.4%) as a white solid. LCMS (ESI) m/z: [M+H]⁺=349.

### Step 3: Preparation of benzyl (3S,4S)-3-((tert-butoxycarbonyl)amino)-4-ethynylpyrrolidine-1-carboxylate

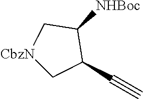 [SMILES: C#C[C@H]1CN(C(=O)OCc2ccccc2)C[C@H]1NC(=O)OC(C)(C)C]

To a mixture of benzyl (3S,4S)-3-((tert-butoxycarbonyl)amino)-4-formylpyrrolidine-1-carboxylate (1.10 g, 3.157 mmol, 1.00 equiv) and K₂CO₃ (1.31 g, 9.471 mmol, 3.00 equiv) in MeOH (30 mL) was added Seyferth-Gilbert reagent (0.91 g, 4.736 mmol, 1.50 equiv) at 0 degrees C. The resulting mixture was stirred for 2 hrs at room temperature. The resulting mixture was diluted with water (50 mL) and extracted with EtOAc (100 mL×3). The combined organic layers were washed with brine (50 mL), and dried over anhydrous sodium sulfate. After filtration, the filtrate was concentrated under reduced pressure to afford benzyl (3S,4S)-3-((tert-butoxycarbonyl)amino)-4-ethynylpyrrolidine-1-carboxylate (1.20 g, crude) as yellow oil. LCMS (ESI) m/z: [M+H]⁺=345.

### Step 4: Preparation of benzyl (3S,4S)-3-amino-4-ethynylpyrrolidine-1-carboxylate

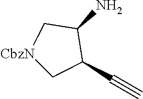 [SMILES: C#C[C@H]1CN(C(=O)OCc2ccccc2)C[C@H]1N]

To a mixture of (3S,4S)-3-((tert-butoxycarbonyl)amino)-4-ethynylpyrrolidine-1-carboxylate (1.20 g, 3.484 mmol, 1.00 equiv) in DCM (15 mL) was added TFA (5 mL). The resulting mixture was stirred for 1 hour at room temperature. The resulting mixture was concentrated under reduced pressure. The residue was purified by reverse phase flash chromatography with the following conditions (column, C₁₈ silica gel; mobile phase, CH₃CN in water, 0% to 50% gradient in 30 min; detector, UV 220 nm) to afford benzyl (3S,4S)-3-amino-4-ethynylpyrrolidine-1-carboxylate (510.0 mg, 53.9%) as yellow solid. LCMS (ESI) m/z: [M+H]⁺=245.

### Step 5: Preparation of benzyl (4bS,7aS)-3-chloro-4b,7,7a,8-tetrahydropyrrolo[3′,4′:4,5]pyrrolo[2,3-c]pyridazine-6(5H)-carboxylate

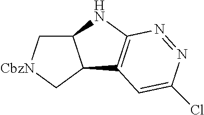 [SMILES: O=C(OCc1ccccc1)N1C[C@@H]2c3cc(Cl)nnc3N[C@@H]2C1]

To a mixture of benzyl (3S,4S)-3-amino-4-ethynylpyrrolidine-1-carboxylate (510.0 mg, 2.088 mmol, 1.00 equiv) and dichloro-1,2,4,5-tetrazine (945.3 mg, 6.264 mmol, 3.00 equiv) in toluene (20 mL) was added DIEA (1349.11 mg, 10.440 mmol, 5.00 equiv). The resulting mixture was stirred overnight at 100 degrees C. The resulting mixture was concentrated under reduced pressure. The residue was purified by reverse phase flash chromatography with the following conditions (column, C₁, silica gel; mobile phase, ACN in water, 0% to 50% gradient in 30 min; detector, UV 254 nm) to afford benzyl (4bS,7aS)-3-chloro-4b,7,7a,8-tetrahydropyrrolo[3′,4′:4,5]pyrrolo[2,3-c]pyridazine-6(5H)-carboxylate (320.0 mg, 41.7%) as a yellow solid. LCMS (ESI) m/z: [M+H]⁺=0.331.

### Step 6: Preparation of benzyl 3-chloro-7,8-dihydropyrrolo[3′,4′:4,5]pyrrolo[2,3-c]pyridazine-6(5H)-carboxylate

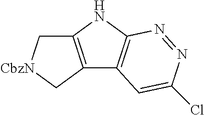 [SMILES: O=C(OCc1ccccc1)N1Cc2[nH]c3nnc(Cl)cc3c2C1]

To a mixture of benzyl (4bS,7aS)-3-chloro-4b,7,7a,8-tetrahydropyrrolo[3′,4′:4,5]pyrrolo[2,3-c]pyridazine-6(5H)-carboxylate (320.0 mg, 0.967 mmol, 1.00 equiv) in DCM (10 mL) was added DMP (820.6 mg, 1.934 mmol, 2.00 equiv). The resulting mixture was stirred for 4 hrs at room temperature. The resulting mixture was filtered, and the filter cake was washed with DCM (3×5 mL). The filtrate was concentrated under reduced pressure. The residue was purified by reverse phase flash chromatography with the following conditions (column, C18 silica gel; mobile phase, CAN in water, 0% to 50% gradient in 30 min; detector, UV 254 nm) to afford benzyl 3-chloro-7,8-dihydropyrrolo[3′,4′:4,5]pyrrolo[2,3-c]pyridazine-6(5H)-carboxylate (125.0 mg, 39.3%) as yellow solid. LCMS (ESI) m/z: [M+H]⁺=329.

### Step 7: Preparation of benzyl 3-(2-hydroxyphenyl)-7,8-dihydropyrrolo[3,4′:4,5]pyrrolo[2,3-c]pyridazine-6(5H)-carboxylate

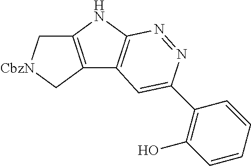 [SMILES: O=C(OCc1ccccc1)N1Cc2[nH]c3nnc(-c4ccccc4O)cc3c2C1]

To a mixture of benzyl 3-chloro-7,8-dihydropyrrolo[3′,4′:4,5]pyrrolo[2,3-c]pyridazine-6(5H)-carboxylate (125.0 mg, 0.380 mmol, 1.00 equiv), 2-hydroxyphenylboronic acid (78.6 mg, 0.570 mmol, 1.5 equiv) and Cs₂CO₃ (371.6 mg, 1.140 mmol, 3.00 equiv) in dioxane (5 mL) and H₂O (1 mL) were added XPhos Pd G3 (64.3 mg, 0.076 mmol, 0.20 equiv), and the resulting mixture was stirred for an hour at 100 degrees C. under an atmosphere of dry nitrogen. The resulting mixture was concentrated under reduced pressure. The residue was purified by reverse phase flash chromatography with the following conditions (column, C18 silica gel; mobile phase, ACN in water, 0% to 50% gradient in 30 min; detector, UV 254 nm) to afford benzyl 3-(2-hydroxyphenyl)-7,8-dihydropyrrolo[3′,4′:4,5]pyrrolo[2,3-c]pyridazine-6(5H)-carboxylate (100.0 mg, 61.2%) as a yellow solid. LCMS (ESI) m/z: [M+H]⁺=387.

### Step 8: Preparation of 2-(5,6,7,8-tetrahydropyrrolo[3′,4′:4,5]pyrrolo[2,3-c]pyridazin-3-yl)phenol (I-106)

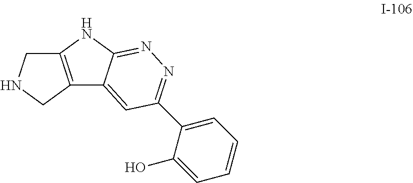 [SMILES: Oc1ccccc1-c1cc2c3c([nH]c2nn1)CNC3]

To a mixture of benzyl 3-(2-hydroxyphenyl)-7,8-dihydropyrrolo[3′,4′:4,5]pyrrolo[2,3-c]pyridazine-6(5H)-carboxylate (100.0 mg, 0.259 mmol, 1.00 equiv) in DCM (5 mL) was added BBr₃ (648.3 mg, 2.590 mmol, 10.00 equiv) at 0 degrees C. The resulting mixture was stirred for 1 h at 0 degrees C. The resulting mixture was quenched with MeOH (1 mL), and then concentrated under reduced pressure. The residue was purified by Prep-HPLC with follow conditions (Column: Gemini-NX C₁₈ AXAI Packed, 21.2*150 mm 5 μm; Mobile Phase A: Water (0.05% FA), Mobile Phase B: MeOH; Flow rate: 25 mL/min; Gradient: 22% B to 68% B in 7 min, 68% B; Wave Length: 220/254 nm;) to afford I-106 (35.0 mg, 53.6%) as a yellow solid. ¹H-NMR (400 MHz, DMSO-d6) δ 14.2 (s, 1H), 8.51 (s, 1H), 8.18 (s, 1H), 8.04 (dd, J=8.0, 1.6 Hz, 1H), 7.29 (ddd, J=8.4, 7.1, 1.6 Hz, 1H), 7.00-6.90 (m, 2H), 4.21 (s, 2H), 4.14 (s, 2H). LCMS (ESI) m/z: [M+H]⁺=253.10.

### Preparation of I-{3-[5-bromo-3-(2-hydroxyphenyl)-7H-pyrrolo[2,3-c]pyridazin-6-yl]azetidin-1-yl}ethanone (I-107)

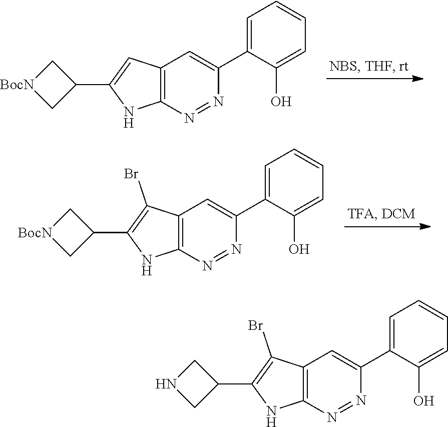 [SMILES: CC(C)(C)OC(=O)N1CC(c2cc3cc(-c4ccccc4O)nnc3[nH]2)C1 ; CC(C)(C)OC(=O)N1CC(c2[nH]c3nnc(-c4ccccc4O)cc3c2Br)C1 ; Oc1ccccc1-c1cc2c(Br)c(C3CNC3)[nH]c2nn1]

### Step 1: Preparation of tert-butyl 3-[5-bromo-3-(2-hydroxyphenyl)-7H-pyrrolo[2,3-c]pyridazin-6-yl]azetidine-1-carboxylate

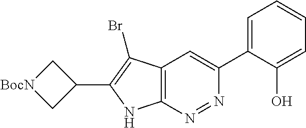 [SMILES: CC(C)(C)OC(=O)N1CC(c2[nH]c3nnc(-c4ccccc4O)cc3c2Br)C1]

To a stirred solution of tert-butyl 3-[3-(2-hydroxyphenyl)-7H-pyrrolo[2,3-c]pyridazin-6-yl]azetidine-1-carboxylate (100 mg, 0.273 mmol, 1.00 equiv) in THF (4.00 mL) was added NBS (38.86 mg, 0.218 mmol, 0.8 equiv) in portions at room temperature. The resulting mixture was stirred for 30 min at room temperature under an atmosphere of dry nitrogen. The resulting mixture was diluted with water (30 mL) and extracted with EtOAc (3×20 mL). The combined organic layers were dried over anhydrous Na₂SO₄. After filtration, the filtrate was concentrated under reduced pressure. The residue was purified by Prep-TLC (PE/EA 1:1) to afford tert-butyl 3-[5-bromo-3-(2-hydroxyphenyl)-7H-pyrrolo[2,3-c]pyridazin-6-yl]azetidine-1-carboxylate (75 mg, 55.54%) as a yellow solid. LCMS (ESI) m/z: [M+H]⁺=445.

### Step 2: Preparation of 2-[6-(azetidin-3-yl)-5-bromo-7H-pyrrolo[2,3-c]pyridazin-3-yl]phenol (I-107)

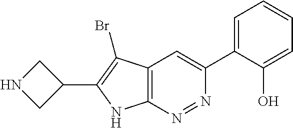 [SMILES: Oc1ccccc1-c1cc2c(Br)c(C3CNC3)[nH]c2nn1]

A solution of tert-butyl 3-[5-bromo-3-(2-hydroxyphenyl)-7H-pyrrolo[2,3-c]pyridazin-6-yl]azetidine-1-carboxylate (70 mg, 0.157 mmol, 1.00 equiv) and TFA (1.00 mL) in DCM (3.00 mL) was stirred for 1 h at room temperature under an atmosphere of dry nitrogen. The resulting mixture was concentrated under reduced pressure to afford crude 2-[6-(azetidin-3-yl)-5-bromo-7H-pyrrolo[2,3-c]pyridazin-3-yl]phenol (70 mg) which was used in the next step directly without further purification. LCMS (ESI) m/z: [M+H]⁺=345.

The following intermediates in Table 17 were prepared in a similar manner as described in the preparation of intermediate I-107.

TABLE 17

| | Intermediate | | LCMS |
|---|---|---|---|
| Structure | No. | Name | (ESI) m/z |
| 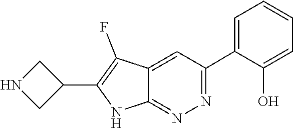 [SMILES: Oc1ccccc1-c1cc2c(F)c(C3CNC3)[nH]c2nn1] | I-108 | 2-(6-(azetidin-3-yl)-5-fluoro- 7H-pyrrolo[2,3-c]pyridazin-3- yl)phenol | 285.1 |
| 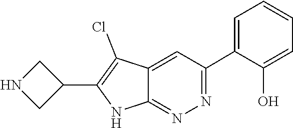 [SMILES: Oc1ccccc1-c1cc2c(Cl)c(C3CNC3)[nH]c2nn1] | I-109 | 2-(6-(azetidin-3-yl)-5-chloro- 7H-pyrrolo[2,3-c]pyridazin-3- yl)phenol | 301 |

### Preparation of 2-[6-(azetidin-3-yl)-7-(difluoromethyl)pyrrolo[2,3-c]pyridazin-3-yl]phenol (I-110)

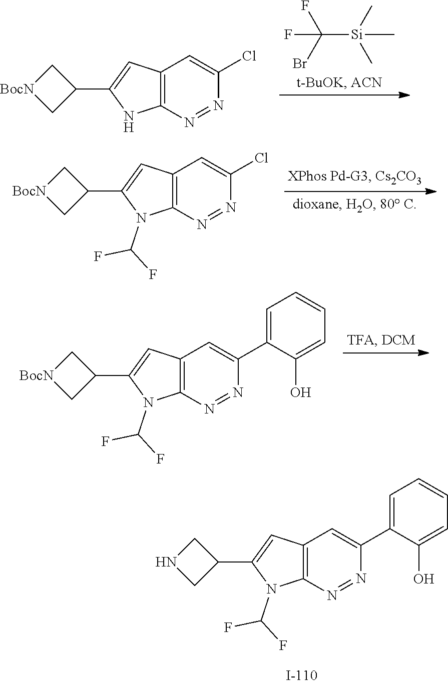

### Step 1: Preparation of tert-butyl 3-[3-chloro-7-(difluoromethyl)pyrrolo[2,3-c]pyridazin-6-yl]azetidine-1-carboxylate

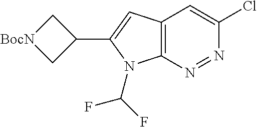 [SMILES: CC(C)(C)OC(=O)N1CC(c2cc3cc(Cl)nnc3n2C(F)F)C1]

To a stirred mixture of tert-butyl 3-{3-chloro-7H-pyrrolo[2,3-c]pyridazin-6-yl}azetidine-1-carboxylate (175 mg, 0.567 mmol, 1.00 equiv) and (bromodifluoromethyl)trimethylsilane (460.44 mg, 2.268 mmol, 4 equiv) in CH₃CN (5 mL) was added t-BuOK (254.39 mg, 2.268 mmol, 4 equiv) in portions at room temperature. The resulting mixture was stirred overnight at room temperature. The desired product could be detected by LCMS. The reaction was quenched by the addition of water (50 mL) at room temperature. The resulting mixture was extracted with EtOAc (3×50 mL). The combined organic layers were dried over anhydrous Na₂SO₄. After filtration, the filtrate was concentrated under reduced pressure. The residue was purified by silica gel column chromatography, eluted with PE/EA (3:1) to afford tert-butyl 3-[3-chloro-7-(difluoromethyl)pyrrolo[2,3-c]pyridazin-6-yl]azetidine-1-carboxylate (130 mg, 58.82%) as a yellow oil. LCMS (ESI) m/z: [M+H]⁺=359.

### Step 2: Preparation of tert-butyl 3-[7-(difluoromethyl)-3-(2-hydroxyphenyl)pyrrolo[2,3-c]pyridazin-6-yl]azetidine-1-carboxylate

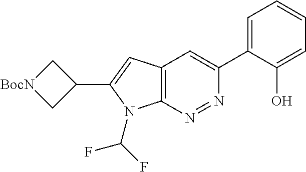 [SMILES: CC(C)(C)OC(=O)N1CC(c2cc3cc(-c4ccccc4O)nnc3n2C(F)F)C1]

To a solution of tert-butyl 3-[3-chloro-7-(difluoromethyl)pyrrolo[2,3-c]pyridazin-6-yl]azetidine-1-carboxylate (130 mg, 0.362 mmol, 1.00 equiv) and 2-hydroxyphenylboronic acid (149.94 mg, 1.086 mmol, 3 equiv) in dioxane (2.5 mL) and H₂O (0.5 mL) were added Cs₂CO₃ (354.18 mg, 1.086 mmol, 3 equiv) and XPhos Pd G3 (61.34 mg, 0.072 mmol, 0.2 equiv). After stirring for 3 hours at 80° C. under a nitrogen atmosphere, the resulting mixture was concentrated under reduced pressure. The residue was purified by silica gel column chromatography, eluted with PE/EA (3:1) to afford tert-butyl 3-[7-(difluoromethyl)-3-(2-hydroxyphenyl)pyrrolo[2,3-c]pyridazin-6-yl]azetidine-1-carboxylate (100 mg, 59.65%) as a yellow solid. LCMS (ESI) m/z: [M+H]⁺=417.

### Step 3: Preparation of 2-[6-(azetidin-3-yl)-7-(difluoromethyl)pyrrolo[2,3-c]pyridazin-3-yl]phenol (I-110)

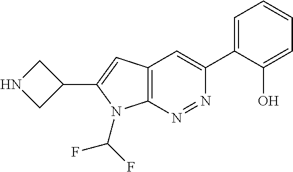 [SMILES: Oc1ccccc1-c1cc2cc(C3CNC3)n(C(F)F)c2nn1]

To a stirred mixture of tert-butyl 3-[7-(difluoromethyl)-3-(2-hydroxyphenyl)pyrrolo[2,3-c]pyridazin-6-yl]azetidine-1-carboxylate (100 mg, 0.240 mmol, 1 equiv) in DCM (3 mL) was added TFA (1 mL) dropwise at room temperature. The resulting mixture was stirred for 1 hour at room temperature. The desired product could be detected by LCMS. The resulting mixture was concentrated under reduced pressure to afford 2-[6-(azetidin-3-yl)-7-(difluoromethyl)pyrrolo[2,3-c]pyridazin-3-yl]phenol (110 mg, 95.80%) as a yellow solid. LCMS (ESI) m/z: [M+H]⁺=317.

### Preparation of (2S,4R)-1-[(2R)-2-(3-{1-[(6R*)-6-amino-3-(2-hydroxyphenyl)-5H,7H,8H,9H-pyridazino[3,4-b]indole-6-carbonyl]piperidin-4-yl}-1,2-oxazol-5-yl)-3-methylbutanoyl]-4-hydroxy-N-[(1S)-1-[4-(4-methyl-1,3-thiazol-5-yl)phenyl]ethyl]pyrrolidine-2-carboxamide (I-111)

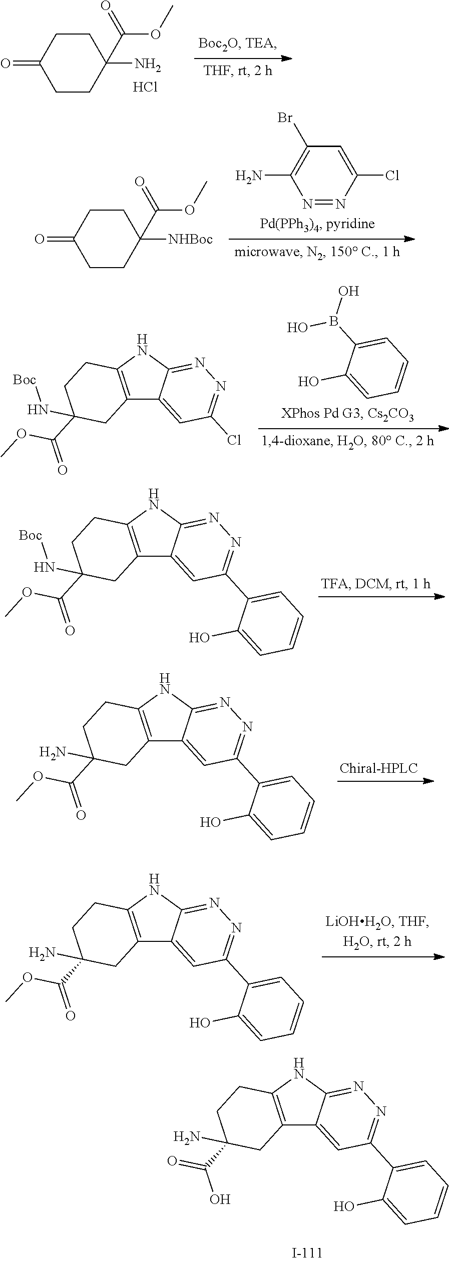

### Step 1: Preparation of methyl 1-[(tert-butoxycarbonyl)amino]-4-oxocyclohexane-1-carboxylate

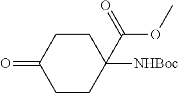 [SMILES: COC(=O)C1(NC(=O)OC(C)(C)C)CCC(=O)CC1]

To a stirred solution of methyl 1-amino-4-oxocyclohexane-1-carboxylate hydrochloride (5.00 g, 24.079 mmol, 1.00 equiv) in THF (50.00 mL) was added (Boc)₂₀ (15.77 g, 72.237 mmol, 3.00 equiv) and TEA (12.18 g, 120.395 mmol, 5.00 equiv) at 0° C. The resulting mixture was stirred for 2 h at room temperature. The reaction was quenched with water at 0° C. The resulting mixture was extracted with DCM (2×100 mL). The combined organic layers were concentrated under reduced pressure. The residue was purified by reverse phase flash chromatography with the following conditions: column, C18 silica gel; mobile phase, MeCN in water, 0 to 100% gradient in 30 min; detector, UV 220/200 nm. This resulted in methyl 1-[(tert-butoxycarbonyl)amino]-4-oxocyclohexane-1-carboxylate (3.8 g, 58.17%) as a white solid. LCMS (ESI) m/z: [M+H]⁺=272.

### Step 2: Preparation of methyl 6-[(tert-butoxycarbonyl)amino]-3-chloro-5H,7H,8H,9H-pyridazino[3,4-b]indole-6-carboxylate

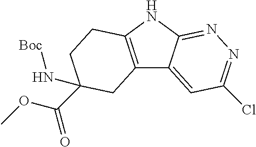 [SMILES: COC(=O)C1(NC(=O)OC(C)(C)C)CCc2[nH]c3nnc(Cl)cc3c2C1]

To a stirred solution of methyl 1-[(tert-butoxycarbonyl)amino]-4-oxocyclohexane-1-carboxylate (3.80 g, 14.743 mmol, 1.00 equiv) in pyridine (15.00 mL) was added 4-bromo-6-chloropyridazin-3-amine (3.07 g, 14.743 mmol, 1.00 equiv) and Pd(PPh₃)₄ (1.70 g, 1.474 mmol, 0.10 equiv) at room temperature. The resulting mixture was stirred for 1 h at 150° C. under nitrogen and microwave atmosphere. The mixture was allowed to cool down to room temperature. The resulting mixture was concentrated under reduced pressure. The residue was purified by reverse phase flash chromatography with the following conditions: column, C18 silica gel; mobile phase, MeCN in water (0.1% FA), 0 to 100% gradient in 30 min; detector, UV 220/200 nm. This resulted in methyl 6-[(tert-butoxycarbonyl)amino]-3-chloro-5H,7H,8H,9H-pyridazino[3,4-b]indole-6-carboxylate (469 mg, 8.35%) as a brown oil. LCMS (ESI) m/z: [M+H]⁺=381.

### Step 3: Preparation of methyl 6-[(tert-butoxycarbonyl)amino]-3-(2-hydroxyphenyl)-5H,7H,8H,9H-pyridazino[3,4-b]indole-6-carboxylate

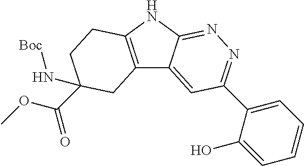 [SMILES: COC(=O)C1(NC(=O)OC(C)(C)C)CCc2[nH]c3nnc(-c4ccccc4O)cc3c2C1]

To a stirred solution of methyl 6-[(tert-butoxycarbonyl)amino]-3-chloro-5H,7H,8H,9H-pyridazino[3,4-b]indole-6-carboxylate (469.00 mg, 1.205 mmol, 1.00 equiv) and 2-hydroxyphenylboronic acid (498.73 mg, 3.615 mmol, 3.00 equiv) in 1,4-dioxane (8.00 mL) and H₂O (2.00 mL) were added XPhos Pd G3 (102.02 mg, 0.121 mmol, 0.10 equiv) and Cs₂CO₃ (1.18 g, 3.622 mmol, 3.00 equiv) at room temperature. The resulting mixture was stirred for 2 h at 80° C. under an atmosphere of dry nitrogen. The mixture was allowed to cool down to room temperature. The residue was purified by reverse phase flash chromatography with the following conditions: column, C18 silica gel; mobile phase, MeCN in water (0.1% FA), 0 to 100% gradient in 30 min; detector, UV 254/220 nm. This resulted in methyl 6-[(tert-butoxycarbonyl)amino]-3-(2-hydroxyphenyl)-5H,7H,8H,9H-pyridazino[3,4-b]indole-6-carboxylate (218 mg, 41.25%) as a brown solid. LCMS (ESI) m/z: [M+H]⁺=439.

### Step 4: Preparation of methyl 6-amino-3-(2-hydroxyphenyl)-5H,7H,8H,9H-pyridazino[3,4-b]indole-6-carboxylate

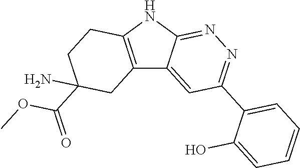 [SMILES: COC(=O)C1(N)CCc2[nH]c3nnc(-c4ccccc4O)cc3c2C1]

To a stirred solution of methyl 6-[(tert-butoxycarbonyl)amino]-3-(2-hydroxyphenyl)-5H,7H,8H,9H-pyridazino[3,4-b]indole-6-carboxylate (218.00 mg, 0.497 mmol, 1.00 equiv) in DCM (10.00 mL) was added TFA (5.00 mL) at room temperature. The resulting mixture was stirred for 1 h at room temperature. The resulting mixture was concentrated under reduced pressure. The resulting mixture was washed with (2×100 mL) of DCM. The combined water layers were concentrated under reduced pressure. This resulted in methyl 6-amino-3-(2-hydroxyphenyl)-5H,7H,8H,9H-pyridazino[3,4-b]indole-6-carboxylate (103 mg, 61.23%) as a yellow solid. LCMS (ESI) m/z: [M+H]⁺=339.

### Step 5: Preparation of methyl (6S)-6-amino-3-(2-hydroxyphenyl)-5H,7H,8H,9H-pyridazino[3,4-b]indole-6-carboxylate

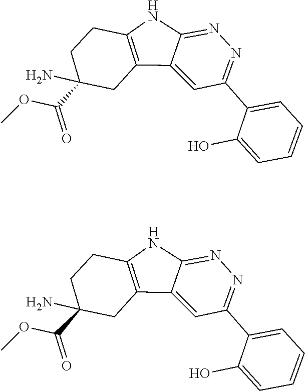 [SMILES: COC(=O)[C@]1(N)CCc2[nH]c3nnc(-c4ccccc4O)cc3c2C1 ; COC(=O)[C@@]1(N)CCc2[nH]c3nnc(-c4ccccc4O)cc3c2C1]

The 6-amino-3-(2-hydroxyphenyl)-5H,7H,8H,9H-pyridazino[3,4-b]indole-6-carboxylate (103.00 mg) was purified by Chiral-Prep-HPLC with the following conditions: Column, CHIRAL ART Amylose-SA, 2*25 cm, 5 um; mobile phase, Hex:MTBE=1:1 (0.5% 2M NH₃-MeOH) and EtOH (hold 30% EtOH in 25 min); Detector, UV 254/220 nm. This resulted in two isomers (34 mg and 30 mg) as yellow solids. LCMS (ESI) m/z: [M+H]⁺=339.

### Step 6: Preparation of (6S)-6-amino-3-(2-hydroxyphenyl)-5H,7H,8H,9H-pyridazino[3,4-b]indole-6-carboxylic acid (I-111)

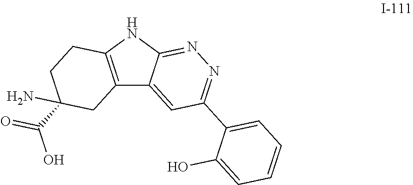 [SMILES: N[C@@]1(C(=O)O)CCc2[nH]c3nnc(-c4ccccc4O)cc3c2C1]

To a stirred solution of methyl (6S)-6-amino-3-(2-hydroxyphenyl)-5H,7H,8H,9H-pyridazino[3,4-b]indole-6-carboxylate (15.00 mg, 0.100 mmol, 1.00 equiv) in THF (1.50 mL) and H₂O (1.50 mL) was added LiOH·H₂O (12.65 mg, 0.300 mmol, 3.00 equiv) at room temperature. The resulting mixture was stirred for 2 h at room temperature. The residue was acidified to pH 6 with aq. HCl solution (1 mol/L). The resulting mixture was extracted with EA (2×100 mL). The combined organic layers were dried over anhydrous Na₂SO₄. After filtration, the filtrate was concentrated under reduced pressure. This resulted in (6S)-6-amino-3-(2-hydroxyphenyl)-5H,7H,8H,9H-pyridazino[3,4-b]indole-6-carboxylic acid (14 mg, crude) as a yellow oil. LCMS (ESI) m/z: [M+H]⁺=325.

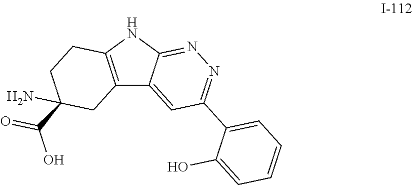 [SMILES: N[C@]1(C(=O)O)CCc2[nH]c3nnc(-c4ccccc4O)cc3c2C1]

Following the same protocol, (6R)-6-amino-3-(2-hydroxyphenyl)-5H,7H,8H,9H-pyridazino[3,4-b]indole-6-carboxylic acid (14 mg, crude) was obtained as a yellow oil. LCMS (ESI) m/z: [M+H]⁺=325.

### Preparation of 2-(3-(4-(2-(4-(3-(2-hydroxyphenyl)-7H-pyrrolo[2,3-c]pyridazin-5-yl)piperidin-1-yl)pyrimidin-5-yl)piperidin-1-yl)isoxazol-5-yl)-3-methylbutanoic acid (I-113)

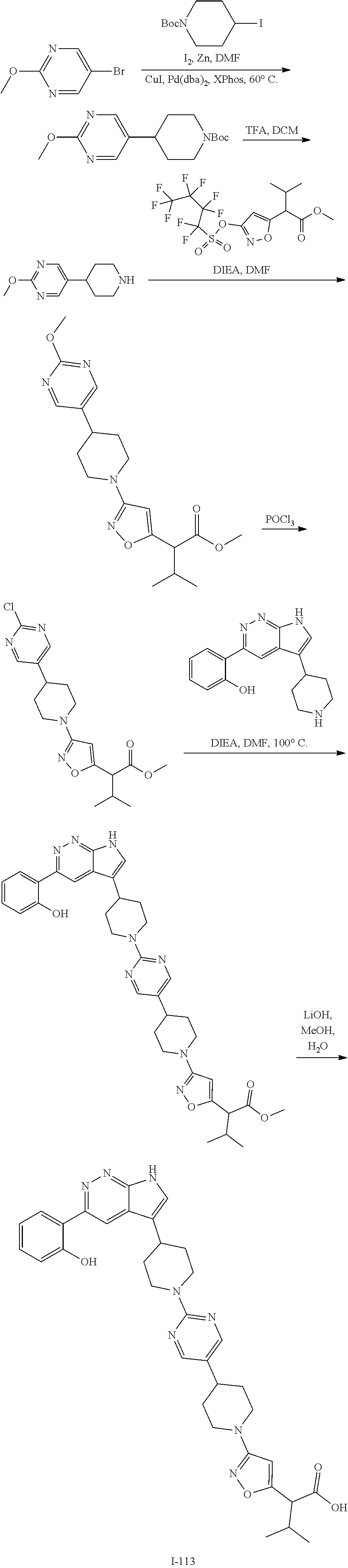

### Step 1: Preparation of tert-butyl 4-(2-methoxypyrimidin-5-yl)piperidine-1-carboxylate

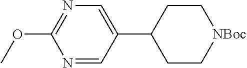 [SMILES: COc1ncc(C2CCN(C(=O)OC(C)(C)C)CC2)cn1]

To a solution of 5-bromo-2-methoxypyrimidine (3.00 g, 15.872 mmol, 1.00 equiv), dtbpy (0.43 g, 1.587 mmol, 0.10 equiv), Mn (1.74 g, 31.744 mmol, 2.00 equiv), KI (2.63 g, 15.872 mmol, 1.00 equiv), pyridine (1.38 g, 17.459 mmol, 1.10 equiv) and NiBr₂ diglyme (0.56 g, 1.587 mmol, 0.10 equiv) in DMA (10 mL). The mixture was stirred for 15 h at 80° C. under a nitrogen atmosphere. The desired product could be detected by LCMS. The reaction mixture was filtered through a short pad of Celite and concentrated in vacuo. The residue was purified by reverse phase flash chromatography with the following conditions (column, 018 silica gel; mobile phase, MeCN in Water (0.1% FA), 10% to 100% gradient in 30 m; detector, UV 254 nm) to afford tert-butyl 4-(2-methoxypyrimidin-5-yl)piperidine-1-carboxylate (1.76 g, 37.80%) as an brown oil. LMS (ESI) m/z [M+H]⁺=294.

### Step 2: Preparation of 2-methoxy-5-(piperidin-4-yl)pyrimidine

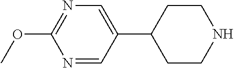

To a solution of tert-butyl 4-(2-methoxypyrimidin-5-yl)piperidine-1-carboxylate (1.70 g, 5.795 mmol, 1.00 equiv) in DCM (10.0 mL) was added TEA (3.0 mL). After stirring for 1 h at room temperature, The desired product could be detected by LCMS. The resulting mixture was concentrated under reduced pressure to afford 2-methoxy-5-(piperidin-4-yl)pyrimidine (860 mg, 76.80%) as a brown oil. LCMS (ESI) m/z [M+H]⁺=194.

### Step 3: Preparation of methyl 2-(3-(4-(2-methoxypyrimidin-5-yl)piperidin-1-yl)isoxazol-5-yl)-3-methylbutanoate

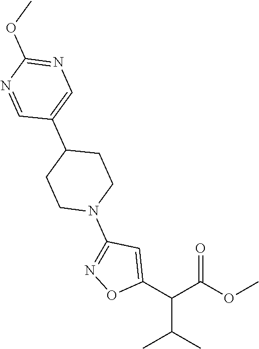 [SMILES: COC(=O)C(c1cc(N2CCC(c3cnc(OC)nc3)CC2)no1)C(C)C]

A mixture of 2-methoxy-5-(piperidin-4-yl)pyrimidine (860.0 mg, 4.450 mmol, 1 equiv), methyl 3-methyl-2-{3-[(1,1,2,2,3,3,4,4,4-nonafluorobutanesulfonyl)oxy]-1,2-oxazol-5-yl}butanoate (3.21 g, 6.675 mmol, 1.50 equiv) and DIEA (1.73 g, 13.350 mmol, 3.00 equiv) in DMF (5 mL) was stirred for 3 h at 130° C. The desired product could be detected by LCMS. The residue was purified by reverse phase flash chromatography with the following conditions (column, C18 silica gel; mobile phase, MeCN in Water (0.1% FA), 10% to 100% gradient in 30 min; detector, UV 254 nm) to afford methyl 2-(3-(4-(2-methoxypyrimidin-5-yl)piperidin-1-yl)isoxazol-5-yl)-3-methylbutanoate (320.0 mg, 19.20%) as an brown solid. LCMS (ESI) m/z [M+H]⁺=375.

### Step 4: Preparation of methyl 2-(3-(4-(2-chloropyrimidin-5-yl)piperidin-1-yl)isoxazol-5-yl)-3-methylbutanoate

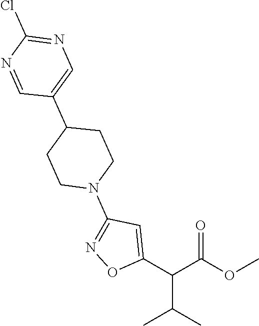 [SMILES: COC(=O)C(c1cc(N2CCC(c3cnc(Cl)nc3)CC2)no1)C(C)C]

A mixture of methyl 2-(3-(4-(2-methoxypyrimidin-5-yl)piperidin-1-yl)isoxazol-5-yl)-3-methylbutanoate (300.0 mg, 0.801 mmol, 1.00 equiv) and POCl₃ (1.23 g, 8.010 mmol, 10.00 equiv) in DMF (2 mL) was stirred for 5 h at 100° C. The desired product could be detected by LCMS. The reaction was quenched with water at 0° C. The residue was purified by reverse phase flash chromatography with the following conditions (column, C18 silica gel; mobile phase, MeCN in Water (0.1% FA), 10% to 100% gradient in 10 min; detector, UV 254 nm) to afford methyl 2-(3-(4-(2-chloropyrimidin-5-yl)piperidin-1-yl)isoxazol-5-yl)-3-methylbutanoate (60.0 mg, 19.77%) as a brown solid. LCMS (ESI) m/z [M+H]⁺=379.

### Step 5: Preparation of methyl 2-(3-(4-(2-(4-(3-(2-hydroxyphenyl)-7H-pyrrolo[2,3-c]pyridazin-5-yl)piperidin-1-yl)pyrimidin-5-yl)piperidin-1-yl)isoxazol-5-yl)-3-methylbutanoate

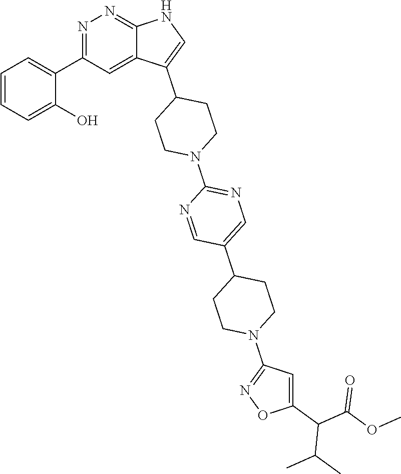 [SMILES: COC(=O)C(c1cc(N2CCC(c3cnc(N4CCC(c5c[nH]c6nnc(-c7ccccc7O)cc56)CC4)nc3)CC2)no1)C(C)C]

A mixture of methyl 2-(3-(4-(2-chloropyrimidin-5-yl)piperidin-1-yl)isoxazol-5-yl)-3-methylbutanoate (60.0 mg, 0.158 mmol, 1.00 equiv), 2-[5-(piperidin-4-yl)-7H-pyrrolo[2,3-c]pyridazin-3-yl]phenol (46.6 mg, 0.158 mmol, 1.00 equiv) and DIEA (61.4 mg, 0.474 mmol, 3.00 equiv) in DMF (2 mL) was stirred for 10 h at 120° C. The desired product could be detected by LCMS. The residue was purified by reverse phase flash chromatography with the following conditions (column, C18 silica gel; mobile phase, MeCN in Water (0.1% FA), 10% to 100% gradient in 40 min; detector, UV 254 nm to afford methyl 2-(3-(4-(2-(4-(3-(2-hydroxyphenyl)-7H-pyrrolo[2,3-c]pyridazin-5-yl)piperidin-1-yl)pyrimidin-5-yl)piperidin-1-yl)isoxazol-5-yl)-3-methylbutanoate (70 mg, 69.41%) as an off-white solid. LCMS (ESI) m/z [M+H]⁺=637.

### Step 6: Preparation of 2-(3-(4-(2-(4-(3-(2-hydroxyphenyl)-7H-pyrrolo[2,3-c]pyridazin-5-yl)piperidin-1-yl)pyrimidin-5-yl)piperidin-1-yl)isoxazol-5-yl)-3-methylbutanoic acid (I-113)

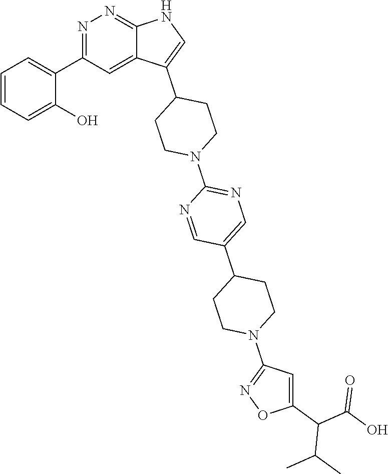 [SMILES: CC(C)C(C(=O)O)c1cc(N2CCC(c3cnc(N4CCC(c5c[nH]c6nnc(-c7ccccc7O)cc56)CC4)nc3)CC2)no1]

A mixture of methyl 2-(3-(4-(2-(4-(3-(2-hydroxyphenyl)-7H-pyrrolo[2,3-c]pyridazin-5-yl)piperidin-1-yl)pyrimidin-5-yl)piperidin-1-yl)isoxazol-5-yl)-3-methylbutanoate (62.0 mg, 0.097 mmol, 1.00 equiv) and LiOH (11.6 mg, 0.485 mmol, 5.00 equiv) in MeOH (1.5 mL) and H₂O (0.3 mL) was stirred for 15 h at room temperature. The desired product could be detected by LCMS. The residue was purified by reverse phase flash chromatography with the following conditions (column, silica gel; mobile phase, MeCN in water, 10% to 100% gradient in 30 min; detector, UV 254 nm) to afford 2-(3-(4-(2-(4-(3-(2-hydroxyphenyl)-7H-pyrrolo[2,3-c]pyridazin-5-yl)piperidin-1-yl)pyrimidin-5-yl)piperidin-1-yl)isoxazol-5-yl)-3-methylbutanoic acid (42 mg, 69.27%) as a yellow solid. LCMS (ESI) m/z [M+H]⁺=623.

### Preparation of 2-(3-{2-[12-(2-hydroxyphenyl)-4,8,10,11-tetraazatricyclo[7.4.0.0\hat{ }{2,7}]trideca-1(9),2(7),10,12-tetraen-4-yl]pyrimidin-5-yl}-1,2-oxazol-5-yl)-3-methylbutanoic acid (I-114)

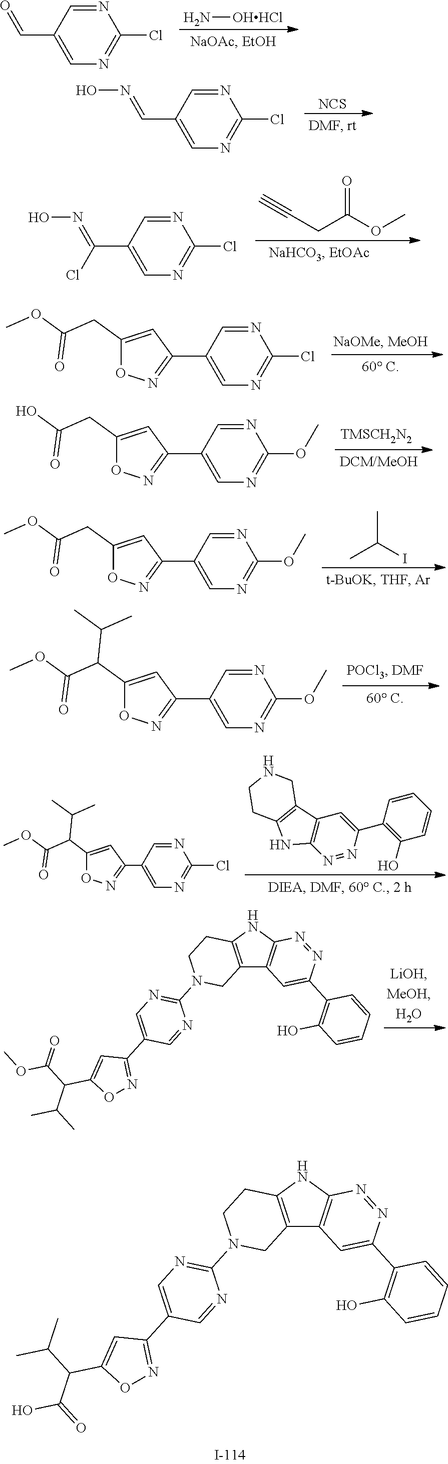

### Step 1: Preparation of (E)-N-[(2-chloropyrimidin-5-yl)methylidene]hydroxylamine

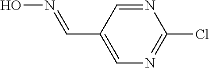

To a stirred solution of 2-chloropyrimidine-5-carbaldehyde (5 g, 35.078 mmol, 1 equiv) and NH₂OH·HCl (4.93 g, 70.945 mmol, 2.02 equiv) in EtOH (250 mL) were added NaOAc (14.48 g, 176.512 mmol, 5.03 equiv) dropwise at room temperature. The resulting mixture was stirred for 2 h at room temperature. The solvent was removed under reduced pressure. The residue was re-solved in EtOAc (500 mL). The organic layers were washed with brine (500 mL), and dried over anhydrous Na₂SO₄. After filtration, the filtrate was concentrated under reduced pressure to afford (E)-N-[(2-chloropyrimidin-5-yl)methylidene]hydroxylamine (4.6 g, crude) as a light yellow solid which was used in the next step directly without further purification. LCMS (ESI) m/z: [M+H]⁺=158.

### Step 2: Preparation of (Z)-2-chloro-N-hydroxypyrimidine-5-carbonimidoyl chloride

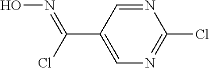 [SMILES: O/N=C(\Cl)c1cnc(Cl)nc1]

A solution of (E)-N-[(2-chloropyrimidin-5-yl)methylidene]hydroxylamine (4.6 g, 29.195 mmol, 1 equiv) and NCS (4.4 g, 32.951 mmol, 1.13 equiv) in DMF (150 mL) was stirred for 2 h at room temperature. The residue was diluted with EtOAc (500 mL). The resulting mixture was washed with of water (3×300 mL), brine (1×300 mL) and the organic phase dried over anhydrous Na₂SO₄. After filtration, the filtrate was concentrated under reduced pressure to afford (Z)-2-chloro-N-hydroxypyrimidine-5-carbonimidoyl chloride (4.8 g, crude) as a yellow solid. LCMS (ESI) m/z: [M+H]⁺=192.

### Step 3: Preparation of methyl 2-[3-(2-chloropyrimidin-5-yl)-1,2-oxazol-5-yl]acetate

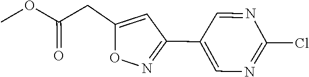 [SMILES: COC(=O)Cc1cc(-c2cnc(Cl)nc2)no1]

A solution of (Z)-2-chloro-N-hydroxypyrimidine-5-carbonimidoyl chloride (4.8 g, 25.00 mmol, 1 equiv) in EtOAc (80 mL) was treated with NaHCO₃ (3 g, 35.712 mmol, 1.43 equiv) for 30 min at 0° C. under an atmosphere of dry nitrogen followed by the addition of methyl but-3-ynoate (2.02 g, 20.591 mmol, 0.82 equiv) in portions at 0° C. The resulting mixture was stirred for additional 12 h at room temperature. The resulting mixture was diluted with water (150 mL) and extracted with EtOAc (2×400 mL). The combined organic layers were washed with brine (1×400 mL), and dried over anhydrous Na₂SO₄. After filtration, the filtrate was concentrated under reduced pressure. The residue was purified by silica gel column chromatography, eluted with PE/EA (3:1) to afford methyl 2-[3-(2-chloropyrimidin-5-yl)-1,2-oxazol-5-yl]acetate (2.5 g, 38.64%) as a light yellow solid. LCMS (ESI) m/z: [M+H]⁺=254.

### Step 4: Preparation of [3-(2-methoxypyrimidin-5-yl)-1,2-oxazol-5-yl]acetic acid

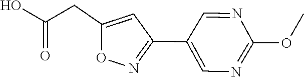 [SMILES: COc1ncc(-c2cc(CC(=O)O)on2)cn1]

A solution of methyl 2-[3-(2-chloropyrimidin-5-yl)-1,2-oxazol-5-yl]acetate (3 g, 11.828 mmol, 1 equiv) and NaOMe (1.92 g, 35.484 mmol, 3.00 equiv) in MeOH (50 mL) was stirred for 1 h at room temperature under an atmosphere of dry nitrogen. The mixture was acidified to pH 6 with HCl (aq.). The residue was dissolved in EtOAc (300 mL). The resulting mixture was washed with water (2×300 mL). The combined organic layers were washed with brine (1×300 mL), and dried over anhydrous Na₂SO₄. After filtration, the filtrate was concentrated under reduced pressure to [3-(2-methoxypyrimidin-5-yl)-1,2-oxazol-5-yl]acetic acid (2.5 g, crude) as a light yellow solid which was used in the next step directly without further purification. LCMS (ESI) m/z: [M+H]⁺=236.

### Step 5: Preparation of methyl 2-[3-(2-methoxypyrimidin-5-yl)-1,2-oxazol-5-yl]acetate

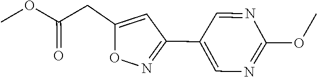

A solution of [3-(2-methoxypyrimidin-5-yl)-1,2-oxazol-5-yl]acetic acid (2.4 g, 10.204 mmol, 1 equiv) and 1-[(trimethylsilyl)methylidene]-1lambda5-diazen-1-ium-2-id-1-ylidene (2.33 g, 20.408 mmol, 2 equiv) in DCM (20 mL) and MeOH (5 mL) was stirred for 30 min at room temperature. The resulting mixture was concentrated under reduced pressure. The residue was purified by silica gel column chromatography, eluted with PE/EA (3:1) to afford methyl 2-[3-(2-methoxypyrimidin-5-yl)-1,2-oxazol-5-yl]acetate (1.2 g, 45.77%) as a white solid. LCMS (ESI) m/z: [M+H]⁺=250.

### Step 6: Preparation of methyl 2-[3-(2-methoxypyrimidin-5-yl)-1,2-oxazol-5-yl]-3-methylbutanoate

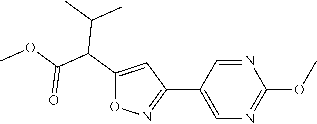

A solution of methyl 2-[3-(2-methoxypyrimidin-5-yl)-1,2-oxazol-5-yl]acetate (2.5 g, 10.031 mmol, 1 equiv) in THF (20 mL) was treated with t-BuOK (1.2 g, 10.694 mmol, 1.07 equiv) for 30 min at 0° C. under an atmosphere of dry nitrogen followed by the addition of 2-iodopropane (1.5 g, 8.824 mmol, 0.88 equiv) dropwise at 0° C. The resulting mixture was stirred for additional 12 h at room temperature. The mixture was acidified to pH 6 with HCl (aq.). The resulting mixture was extracted with EtOAc (2×200 mL). The combined organic layers were washed with brine (2×200 mL), and dried over anhydrous Na₂SO₄. After filtration, the filtrate was concentrated under reduced pressure. The residue was purified by silica gel column chromatography, eluted with PE/EA (3:1) to afford methyl 2-[3-(2-methoxypyrimidin-5-yl)-1,2-oxazol-5-yl]-3-methylbutanoate (310 mg, 10.08%) as a yellow oil. LCMS (ESI) m/z: [M+H]⁺=292.

### Step 7: Preparation of methyl 2-[3-(2-chloropyrimidin-5-yl)-1,2-oxazol-5-yl]-3-methylbutanoate

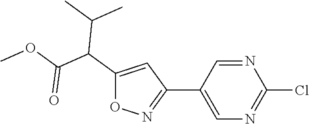 [SMILES: COC(=O)C(c1cc(-c2cnc(Cl)nc2)no1)C(C)C]

A solution of methyl methyl 2-[3-(2-methoxypyrimidin-5-yl)-1,2-oxazol-5-yl]-3-methylbutanoate (200 mg, 0.687 mmol, 1 equiv) and POCl₃ (1.9 mL, 20.61 mmol, 30 equiv) in DMF (1.5 mL) was stirred for 3 h at room temperature under an atmosphere of dry nitrogen. The residue was dissolved in EtOAc (100 mL). The resulting mixture was washed with 2×100 mL of brine, and dried over anhydrous Na₂SO₄. After filtration, the filtrate was concentrated under reduced pressure to afford methyl 2-[3-(2-chloropyrimidin-5-yl)-1,2-oxazol-5-yl]-3-methylbutanoate (160 mg, crude) as a brown oil which was used in the next step directly without further purification. LCMS (ESI) m/z: [M+H]⁺=296.

### Step 8: Preparation of methyl 2-(3-{2-[12-(2-hydroxyphenyl)-4,8,10,11-tetraazatricyclo[7.4.0.0\hat{ }{2,7}]trideca-1(9),2(7),10,12-tetraen-4-yl]pyrimidin-5-yl}-1,2-oxazol-5-yl)-3-methylbutanoate

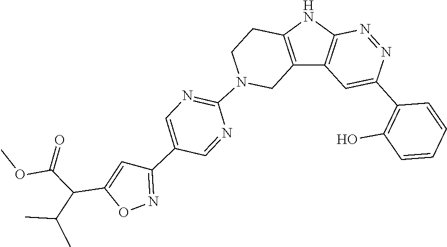 [SMILES: COC(=O)C(c1cc(-c2cnc(N3CCc4[nH]c5nnc(-c6ccccc6O)cc5c4C3)nc2)no1)C(C)C]

To a stirred mixture of methyl 2-[3-(2-chloropyrimidin-5-yl)-1,2-oxazol-5-yl]-3-methylbutanoate (270 mg, 0.913 mmol, 1 equiv) and 2-{4,8,10,11-tetraazatricyclo[7.4.0.0\hat{ }{2,7}]trideca-1(9),2(7),10,12-tetraen-12-yl}phenol (243.14 mg, 0.913 mmol, 1 equiv) in DMF (5 mL) was added DIEA (354.02 mg, 2.739 mmol, 3 equiv) dropwise at room temperature under an atmosphere of dry nitrogen. The resulting mixture was stirred for additional 2 h at 60° C. The resulting mixture was extracted with EtOAc (2×50 mL). The combined organic layers were washed with brine (2×50 mL), and dried over anhydrous Na₂SO₄. After filtration, the filtrate was concentrated under reduced pressure. The residue was purified by reverse phase flash chromatography with the following conditions: column, C18 silica gel; mobile phase, MeCN in Water (0.1% FA), 0% to 100% gradient in 35 min; detector, UV 254/220 nm. This resulted in methyl 2-(3-{2-[12-(2-hydroxyphenyl)-4,8,10,11-tetraazatricyclo[7.4.0.0\hat{ }{2,7}]trideca-1(9),2(7),10,12-tetraen-4-yl]pyrimidin-5-yl}-1,2-oxazol-5-yl)-3-methylbutanoate (82 mg, 17.09%) as a yellow solid. LCMS (ESI) m/z: [M+H]⁺=526.

### Step 9: Preparation of 2-(3-{2-[12-(2-hydroxyphenyl)-4,8,10,11-tetraazatricyclo[7.4.0.0\hat{ }{2,7}]trideca-1(9),2(7),10,12-tetraen-4-yl]pyrimidin-5-yl}-1,2-oxazol-5-yl)-3-methylbutanoic acid (I-114)

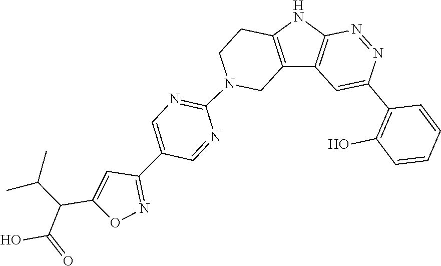 [SMILES: CC(C)C(C(=O)O)c1cc(-c2cnc(N3CCc4[nH]c5nnc(-c6ccccc6O)cc5c4C3)nc2)no1]

To a stirred solution of methyl 2-(3-{2-[12-(2-hydroxyphenyl)-4,8,10,11-tetraazatricyclo[7.4.0.0\hat{ }{2,7}] trideca-1(9),2(7),10,12-tetraen-4-yl]pyrimidin-5-yl}-1,2-oxazol-5-yl)-3-methylbutanoate (82 mg, 0.156 mmol, 1 equiv) in MeOH (3.00 mL) and H₂O (3.00 mL) was added LiOH·H₂O (65.47 mg, 1.560 mmol, 10 equiv) at room temperature. The resulting mixture was stirred for 2 h at room temperature. The residue was acidified to pH 6 with HCl (1 M, aq.). The resulting mixture was extracted with CHCl₃/2-propanol (3:1) (2×50 mL). The combined organic layers were washed with water (2×50 mL), and dried over anhydrous Na₂SO₄. After filtration, the filtrate was concentrated under reduced pressure. This resulted in 2-(3-{2-[12-(2-hydroxyphenyl)-4,8,10,11-tetraazatricyclo[7.4.0.0\hat{ }{2,7}]trideca-1(9),2(7),10,12-tetraen-4-yl]pyrimidin-5-yl}-1,2-oxazol-5-yl)-3-methylbutanoic acid (80 mg, crude) as a yellow oil which was used in the next step directly without further purification. LCMS (ESI) m/z: [M+H]⁺=512.

### Preparation of 12-(2-hydroxyphenyl)-4,8,10,11-tetraazatricyclo[7.4.0.0\hat{ }{2,7}] trideca-1(9),2(7),10,12-tetraene-4-carboximidamide (I-115)

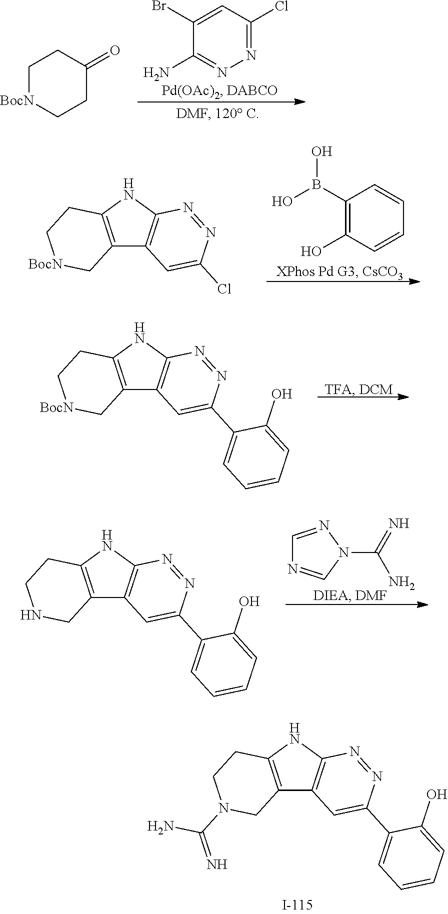

### Step 1: Preparation of tert-butyl 12-chloro-4,8,10,11-tetraazatricyclo[7.4.0.0\hat{ }{2,7}]trideca-1(9),2(7),10,12-tetraene-4-carboxylate

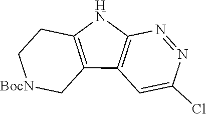 [SMILES: CC(C)(C)OC(=O)N1CCc2[nH]c3nnc(Cl)cc3c2C1]

A solution of tert-butyl 4-oxopiperidine-1-carboxylate (7 g, 35.132 mmol, 1 equiv), 4-bromo-6-chloropyridazin-3-amine (8.79 g, 42.158 mmol, 1.2 equiv), Pd(OAc)₂ (1.58 g, 7.026 mmol, 0.2 equiv) and 1,4-diazabicyclo[2,2,2]octane (11.82 g, 105.396 mmol, 3 equiv) in DMF (100 mL) was stirred for 8 hours at 120° C. under an atmosphere of dry nitrogen. The resulting mixture was filtered, the filter cake was washed with CH₃CN (3×60 mL). The filtrate was concentrated under reduced pressure. The residue was purified by reverse phase flash chromatography with the following conditions: column, silica gel; mobile phase, CH₃CN in water, 0% to 100% gradient in 40 min; detector, UV 254 nm to afford tert-butyl 12-chloro-4,8,10,11-tetraazatricyclo[7.4.0.0\hat{ }{2,7}]trideca-1(9),2(7),10,12-tetraene-4-carboxylate (419 mg, 3.86%) as a brown solid. LCMS (ESI) m/z: [M+H]⁺=309.

### Step 2: Preparation of tert-butyl 12-(2-hydroxyphenyl)-4,8,10,11-tetraazatricyclo[7.4.0.0\hat{ }{2,7}]trideca-1(9),2(7),10,12-tetraene-4-carboxylate

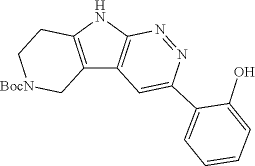 [SMILES: CC(C)(C)OC(=O)N1CCc2[nH]c3nnc(-c4ccccc4O)cc3c2C1]

A solution of tert-butyl 12-chloro-4,8,10,11-tetraazatricyclo[7.4.0.0\hat{ }{2,7}]trideca-1(9),2(7),10,12-tetraene-4-carboxylate (210 mg, 0.680 mmol, 1 equiv), 2-hydroxyphenylboronic acid (140.71 mg, 1.020 mmol, 1.5 equiv), XPhos Pd G3 (115.14 mg, 0.136 mmol, 0.2 equiv) and Cs₂CO₃ (664.79 mg, 2.040 mmol, 3 equiv) in 1,4-dioxane (5 mL) and H₂O (1 mL) was stirred for 2 hours at 100° C. under an atmosphere of dry nitrogen. The resulting mixture was filtered, the filter cake was washed with EA (3×10 mL). The filtrate was concentrated under reduced pressure. The residue was purified by reverse phase flash chromatography with the following conditions: column, silica gel; mobile phase, CH₃CN in water, 0% to 100% gradient in 40 min; detector, UV 254 nm to afford tert-butyl 12-(2-hydroxyphenyl)-4,8,10,11-tetraazatricyclo[7.4.0.0\hat{ }{2,7}]trideca-1(9),2(7),10,12-tetraene-4-carboxylate (157 mg, 62.91%) as a yellow solid. LCMS (ESI) m/z: [M+H]⁺=367.

### Step 3: Preparation of 2-{4,8,10,11-tetraazatricyclo[7.4.0.0\hat{ }{2,7}]trideca-1(9),2(7),10,12-tetraen-12-yl}phenol

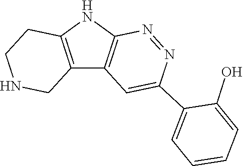 [SMILES: Oc1ccccc1-c1cc2c3c([nH]c2nn1)CCNC3]

A solution of tert-butyl 12-(2-hydroxyphenyl)-4,8,10,11-tetraazatricyclo[7.4.0.0\hat{ }{2,7}]trideca-1(9),2(7),10,12-tetraene-4-carboxylate (157 mg, 0.428 mmol, 1 equiv) and TFA (2 mL) in DCM (2 mL) was stirred for one hour at room temperature. The resulting mixture was concentrated under vacuum. The residue was purified by reverse phase flash chromatography with the following conditions: column, silica gel; mobile phase, CH₃CN in water, 0% to 100% gradient in 40 min; detector, UV 254 nm to afford 2-{4,8,10,11-tetraazatricyclo[7.4.0.0\hat{ }{2,7}]trideca-1(9),2(7),10,12-tetraen-12-yl}phenol (97 mg, 84.88%) as a brown solid. LCMS (ESI) m/z: [M+H]⁺=267.

### Step 4: Preparation of 12-(2-hydroxyphenyl)-4,8,10,11-tetraazatricyclo[7.4.0.0\hat{ }{2,7}]trideca-1(9),2(7),10,12-tetraene-4-carboximidamide (I-115)

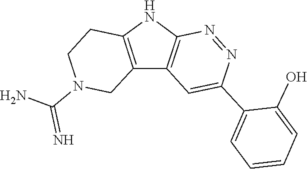 [SMILES: N=C(N)N1CCc2[nH]c3nnc(-c4ccccc4O)cc3c2C1]

A solution of 2-{4,8,10,11-tetraazatricyclo[7.4.0.0\hat{ }{2,7}]trideca-1(9),2(7),10,12-tetraen-12-yl}phenol (35 mg, 0.131 mmol, 1 equiv), 1,2,4-triazole-1-carboximidamide (17.52 mg, 0.157 mmol, 1.2 equiv) and DIEA (84.93 mg, 0.655 mmol, 5 equiv) in DMF (1 mL) was stirred for 12 hours at room temperature under an atmosphere of dry nitrogen. The crude product was purified by Prep-HPLC with the following conditions (Column: SunFire Prep C18 OBD Column, 19*150 mm, 5 μm; Mobile Phase A: water (0.05% FA), Mobile Phase B: CH₃CN; Flow rate: 25 mL/min; Gradient: 2% B to 18% B in 7 min, 18% B; to afford I-115 (22.3 mg, 55.09%) as a yellow solid. ¹H NMR (300 MHz, DMSO-d6) δ 13.95 (s, 1H), 8.58-8.26 (m, 5H), 8.00 (d, J=7.8 Hz, 1H), 7.36-7.25 (m, 1H), 7.03-6.92 (m, 2H), 4.74 (s, 2H), 3.86 (t, J=3.2 Hz, 2H), 3.05 (s, J=4.7 Hz, 2H). LCMS (ESI) m/z: [M+H]⁺=309.10.

### Preparation of 2-{3-[4-(2-{4-[3-(2-hydroxyphenyl)-7H-pyrrolo[2,3-c]pyridazin-5-yl]piperidin-1-yl}pyrimidin-5-yl)cyclohexyl]-1,2-oxazol-5-yl}-3-methylbutanoic acid (I-116)

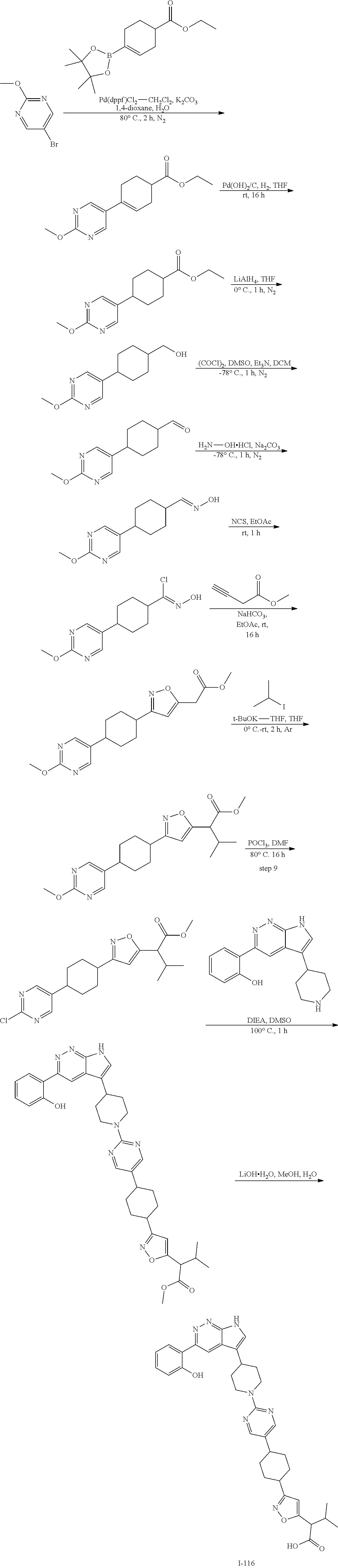

### Step 1: Preparation of ethyl 4-(2-methoxypyrimidin-5-yl)cyclohex-3-ene-1-carboxylate

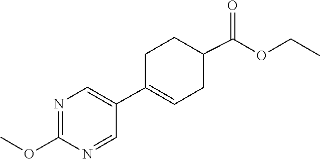 [SMILES: CCOC(=O)C1CC=C(c2cnc(OC)nc2)CC1]

To a stirred solution of 5-bromo-2-methoxypyrimidine (6.00 g, 31.746 mmol, 1.00 equiv) and ethyl 4-(4,4,5,5-tetramethyl-1,3,2-dioxaborolan-2-yl)cyclohex-3-ene-1-carboxylate (8.89 g, 31.746 mmol, 1.00 equiv) in 1,4-dioxane (40.00 mL) and H₂O (10.00 mL) were added Pd(dppf)Cl₂—CH₂Cl₂ (2.60 g, 3.175 mmol, 0.10 equiv) and K₂CO₃ (13.14 g, 95.238 mmol, 3.00 equiv) at room temperature. The resulting mixture was stirred for 2 h at 80° C. under an atmosphere of dry nitrogen. The mixture was allowed to cool down to room temperature. The resulting mixture was concentrated under reduced pressure. The residue was purified by silica gel column chromatography, eluted with PE/EA (1/2) to afford ethyl 4-(2-methoxypyrimidin-5-yl)cyclohex-3-ene-1-carboxylate (7.20 g, 86.54%) as a yellow oil. LCMS (ESI) m/z: [M+H]⁺=263.

### Step 2: Preparation of ethyl 4-(2-methoxypyrimidin-5-yl)cyclohexane-1-carboxylate

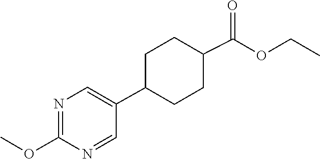 [SMILES: CCOC(=O)C1CCC(c2cnc(OC)nc2)CC1]

To a stirred solution of ethyl 4-(2-methoxypyrimidin-5-yl)cyclohex-3-ene-1-carboxylate (7.20 g, 27.481 mmol, 1.00 equiv) in THF (50.00 mL) was added Pd(OH)₂/C (3.85 g, 27.481 mmol, 1.00 equiv) at room temperature. The resulting mixture was stirred for 16 h at room temperature under hydrogen atmosphere. The resulting mixture was filtered, the filter cake was washed with THF. The filtrate was concentrated under reduced pressure. This resulted in ethyl 4-(2-methoxypyrimidin-5-yl)cyclohexane-1-carboxylate (6.00 g, 82.76%) as a grey oil. LCMS (ESI) m/z: [M+H]⁺=265.

### Step 3: Preparation of [4-(2-methoxypyrimidin-5-yl)cyclohexyl]methanol

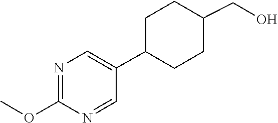 [SMILES: COc1ncc(C2CCC(CO)CC2)cn1]

To a stirred solution of ethyl 4-(2-methoxypyrimidin-5-yl)cyclohexane-1-carboxylate (6.00 g, 22.727 mmol, 1.00 equiv) in THF (50.00 mL) was added LiAlH₄ (9.09 mL, 22.727 mmol, 1.00 equiv, 2.50 mol/L) at 0° C. under an atmosphere of dry nitrogen. The resulting mixture was stirred for 1 h at 0° C. under an atmosphere of dry nitrogen. The reaction was quenched with water at 0° C. The resulting mixture was extracted with DCM (2×100 mL). The combined organic layers were washed with saturated brine (1×100 mL), and dried over anhydrous Na₂SO₄. After filtration, the filtrate was concentrated under reduced pressure. This resulted in [4-(2-methoxypyrimidin-5-yl)cyclohexyl]methanol (2.70 g, 53.46%) as a yellow oil. LCMS (ESI) m/z: [M+H]⁺=223.

### Step 4: Preparation of 4-(2-methoxypyrimidin-5-yl)cyclohexane-1-carbaldehyde

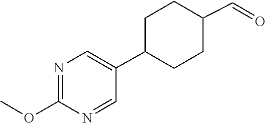 [SMILES: COc1ncc(C2CCC(C=O)CC2)cn1]

To a stirred solution of (COCl)₂ (4.63 g, 36.486 mmol, 3.00 equiv) in DCM (30.00 mL) was added DMSO (3.79 g, 48.649 mmol, 4.00 equiv) at −70° C. under an atmosphere of dry nitrogen. The resulting mixture was stirred for 30 min at −78° C. under an atmosphere of dry nitrogen. To the above mixture was added [4-(2-methoxypyrimidin-5-yl)cyclohexyl]methanol (2.70 g, 12.162 mmol, 1.00 equiv) at −70° C. under an atmosphere of dry nitrogen. The resulting mixture was stirred for additional 30 min at −70° C. under an atmosphere of dry nitrogen. To the above mixture was added Et₃N (6.14 g, 60.811 mmol, 5.00 equiv) at −70° C. under an atmosphere of dry nitrogen. The resulting mixture was stirred for additional 1 h at room temperature. The reaction was quenched by the addition of water (100 mL) at 0° C. The resulting mixture was extracted with DCM (2×100 mL). The combined organic layers were washed with brine (2×100 mL), and dried over anhydrous Na₂SO₄. After filtration, the filtrate was concentrated under reduced pressure. This resulted in 4-(2-methoxypyrimidin-5-yl)cyclohexane-1-carbaldehyde (2.40 g, 89.55%) as a yellow solid. LCMS (ESI) m/z: [M+H]⁺=221.

### Step 5: Preparation of (E)-N-{[4-(2-methoxypyrimidin-5-yl)cyclohexyl]methylidene}hydroxylamine

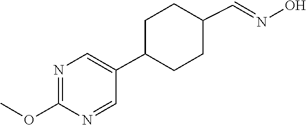 [SMILES: COc1ncc(C2CCC(/C=N/O)CC2)cn1]

To a stirred solution of hydroxylamine hydrochloride (2.46 g, 35.412 mmol, 3.00 equiv) in MeOH (8.00 mL) and H₂O (24.00 mL) was added Na₂CO₃ (3.75 g, 35.412 mmol, 3.00 equiv) at 0° C. To the above mixture was added 4-(2-methoxypyrimidin-5-yl)cyclohexane-1-carbaldehyde 2.40 g, 11.804 mmol, 1.00 equiv) at 0° C. The resulting mixture was stirred for additional 1 h at room temperature. The reaction was quenched with water at 0° C. The resulting mixture was extracted with EA (2×200 mL). The combined organic layers were washed with saturated brine (1×200 mL), and dried over anhydrous Na₂SO₄. After filtration, the filtrate was concentrated under reduced pressure. This resulted in (E)-N-{[4-(2-methoxypyrimidin-5-yl)cyclohexyl]methylidene}hydroxylamine (2.80 g, crude) as a yellow oil. LCMS (ESI) m/z: [M+H]⁺=236.

### Step 6: Preparation of (Z)—N-hydroxy-4-(2-methoxypyrimidin-5-yl)cyclohexane-1-carbonimidoyl chloride

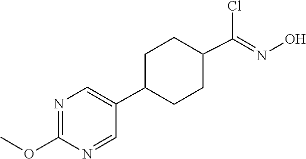 [SMILES: COc1ncc(C2CCC(/C(Cl)=N/O)CC2)cn1]

To a stirred solution of (E)-N-{[4-(2-methoxypyrimidin-5-yl)cyclohexyl]methylidene}hydroxylamine (2.80 g, 11.900 mmol, 1.00 equiv) in EA (30.00 mL) was added NCS (1.91 g, 14.280 mmol, 1.20 equiv) at 0° C. The resulting mixture was stirred for 1 h at room temperature. The resulting mixture was used in the next step directly without further purification. LCMS (ESI) m/z: [M+H]⁺=270.

### Step 7: Preparation of methyl 2-{3-[4-(2-methoxypyrimidin-5-yl)cyclohexyl]-1,2-oxazol-5-yl}acetate

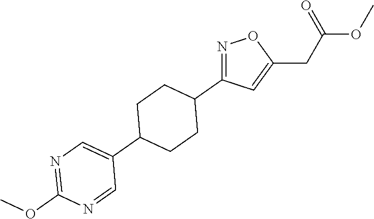

To a stirred solution of methyl but-3-ynoate (4.66 g, 47.456 mmol, 4.00 equiv) in EA (30.00 mL) was added NaHCO₃ (2.99 g, 35.592 mmol, 3.00 equiv) at 0° C. To the above mixture was added (Z)—N-hydroxy-4-(2-methoxypyrimidin-5-yl)cyclohexane-1-carbonimidoyl chloride (3.20 g, 11.864 mmol, 1.00 equiv) at 0° C. The resulting mixture was stirred for additional 16 h at room temperature. The reaction was quenched with water at 0° C. The resulting mixture was extracted with EA (2×200 mL). The combined organic layers were concentrated under reduced pressure. The residue was purified by reverse phase flash chromatography with the following conditions: column, C18 silica gel; mobile phase, MeCN in water (0.1% FA), 0 to 100% gradient in 30 min; detector, UV 254/220 nm. This resulted in intermediate methyl 2-{3-[4-(2-methoxypyrimidin-5-yl)cyclohexyl]-1,2-oxazol-5-yl}acetate (2.70 g, 68.68%) as a brown oil. LCMS (ESI) m/z: [M+H]⁺=332.

### Step 8: Preparation of methyl 2-{3-[4-(2-methoxypyrimidin-5-yl)cyclohexyl]-1,2-oxazol-5-yl}-3-methylbutanoate

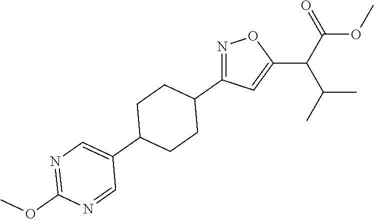

To a stirred solution of methyl 2-{3-[4-(2-methoxypyrimidin-5-yl)cyclohexyl]-1,2-oxazol-5-yl}acetate (2.70 g, 8.132 mmol, 1.00 equiv) in THF (30.00 mL) was added t-BuOK (24.40 mL, 24.398 mmol, 3.00 equiv, 1.0 mol/L) at 0° C. under an atmosphere of dry nitrogen. The resulting mixture was stirred for 30 min at 0° C. under an atmosphere of dry nitrogen. To the above mixture was added 2-iodopropane (4.15 g, 24.398 mmol, 3.00 equiv) at 0° C. The resulting mixture was stirred for additional 2 h at room temperature. The reaction was quenched with water (100 mL) at 0° C. The residue was acidified to pH 3 with HCl (1 mol/L). The resulting mixture was extracted with DCM (2×200 mL). The combined organic layers were concentrated under reduced pressure. The residue was dissolved in DCM (16.00 mL) and MeOH (4.00 mL). To the above mixture was added CH₂N₂ (1.02 g, 24.396 mmol, 3.00 equiv) at 0° C. The resulting mixture was stirred for additional 1 h at room temperature. The reaction was quenched with water at 0° C. The resulting mixture was extracted with DCM (2×200 mL). The combined organic layers were concentrated under reduced pressure. The residue was purified by reverse phase flash chromatography with the following conditions: column, C18 silica gel; mobile phase, MeCN in water (0.1% FA), 0 to 100% gradient in 30 min; detector, UV 254/220 nm. This resulted in methyl 2-{3-[4-(2-methoxypyrimidin-5-yl)cyclohexyl]-1,2-oxazol-5-yl}-3-methylbutanoate (795 mg, 38.26%) as a brown oil. LCMS (ESI) m/z: [M+H]⁺=374.

### Step 9: Preparation of methyl 2-{3-[4-(2-chloropyrimidin-5-yl)cyclohexyl]-1,2-oxazol-5-yl}-3-methylbutanoate

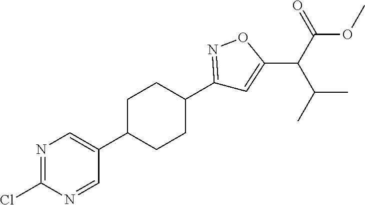 [SMILES: COC(=O)C(c1cc(C2CCC(c3cnc(Cl)nc3)CC2)no1)C(C)C]

To a stirred solution of methyl 2-{3-[4-(2-methoxypyrimidin-5-yl)cyclohexyl]-1,2-oxazol-5-yl}-3-methylbutanoate (795.00 mg, 2.129 mmol, 1.00 equiv) in DMF (5.00 mL) was added POCl₃ (979.15 mg, 6.387 mmol, 3.00 equiv) at room temperature. The resulting mixture was stirred for 16 h at 80° C. The mixture was allowed to cool down to room temperature. The residue was purified by reverse phase flash chromatography with the following conditions: column, C18 silica gel; mobile phase, MeCN in water (0.1% FA), 0 to 100% gradient in 30 min; detector, UV 254/220 nm. This resulted in methyl 2-{3-[4-(2-chloropyrimidin-5-yl)cyclohexyl]-1,2-oxazol-5-yl}-3-methylbutanoate (154 mg, 19.14%) as a brown oil. LCMS (ESI) m/z: [M+H]⁺=378.

### Step 10: Preparation of methyl 2-{3-[4-(2-{4-[3-(2-hydroxyphenyl)-7H-pyrrolo[2,3-c]pyridazin-5-yl]piperidin-1-yl}pyrimidin-5-yl)cyclohexyl]-1,2-oxazol-5-yl}-3-methylbutanoate

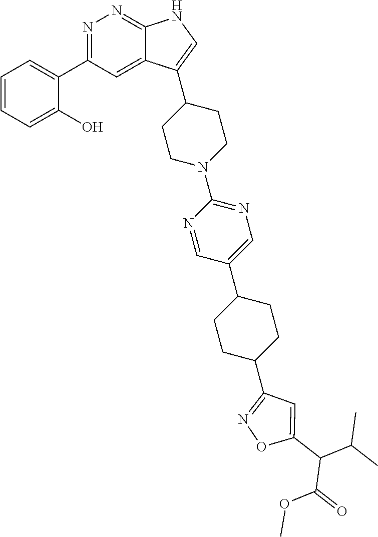 [SMILES: COC(=O)C(c1cc(C2CCC(c3cnc(N4CCC(c5c[nH]c6nnc(-c7ccccc7O)cc56)CC4)nc3)CC2)no1)C(C)C]

To a stirred solution of methyl 2-{3-[4-(2-chloropyrimidin-5-yl)cyclohexyl]-1,2-oxazol-5-yl}-3-methylbutanoate (80.00 mg, 0.212 mmol, 1.00 equiv) in DMSO (2.00 mL) was added 2-[5-(piperidin-4-yl)-7H-pyrrolo[2,3-c]pyridazin-3-yl]phenol (62.32 mg, 0.212 mmol, 1.00 equiv) and DIEA (136.82 mg, 1.060 mmol, 5.00 equiv) at room temperature. The resulting mixture was stirred for 1 h at 100° C. The mixture was allowed to cool down to room temperature. The residue was purified by reverse phase flash chromatography with the following conditions: column, C18 silica gel; mobile phase, MeCN in water (0.1% FA), 0 to 100% gradient in 30 min; detector, UV 254/220 nm. This resulted in methyl 2-{3-[4-(2-{4-[3-(2-hydroxyphenyl)-7H-pyrrolo[2,3-c]pyridazin-5-yl]piperidin-1-yl}pyrimidin-5-yl)cyclohexyl]-1,2-oxazol-5-yl}-3-methylbutanoate (61 mg, 45.32%) as a yellow oil. LCMS (ESI) m/z: [M+H]⁺=636.

### Step 11: Preparation of 2-{3-[4-(2-{4-[3-(2-hydroxyphenyl)-7H-pyrrolo[2,3-c]pyridazin-5-yl]piperidin-1-yl}pyrimidin-5-yl)cyclohexyl]-1,2-oxazol-5-yl}-3-methylbutanoic acid

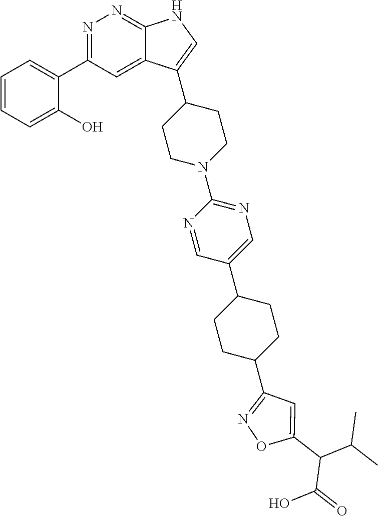 [SMILES: CC(C)C(C(=O)O)c1cc(C2CCC(c3cnc(N4CCC(c5c[nH]c6nnc(-c7ccccc7O)cc56)CC4)nc3)CC2)no1]

To a stirred solution of methyl 2-{3-[4-(2-{4-[3-(2-hydroxyphenyl)-7H-pyrrolo[2,3-c]pyridazin-5-yl]piperidin-1-yl}pyrimidin-5-yl)cyclohexyl]-1,2-oxazol-5-yl}-3-methylbutanoate (61.00 mg, 0.093 mmol, 1.00 equiv) in MeOH (2.00 mL) and H₂O (2.00 mL) was added LiOH·H₂O (11.68 mg, 0.279 mmol, 3.00 equiv) at room temperature. The resulting mixture was stirred for 1 h at room temperature. The residue was acidified to pH 3 with HCl aq. solution (1 mol/L). The resulting mixture was concentrated under reduced pressure. This resulted in 2-{3-[4-(2-{4-[3-(2-hydroxyphenyl)-7H-pyrrolo[2,3-c]pyridazin-5-yl]piperidin-1-yl}pyrimidin-5-yl)cyclohexyl]-1,2-oxazol-5-yl}-3-methylbutanoic acid (60 mg, crude) as a yellow solid. LCMS (ESI) m/z: [M+H]⁺=622.

### Preparation of 5-(azetidin-3-yl)-3-(2-hydroxyphenyl)-7H-imidazo[4,5-c]pyridazin-6-one (I-117)

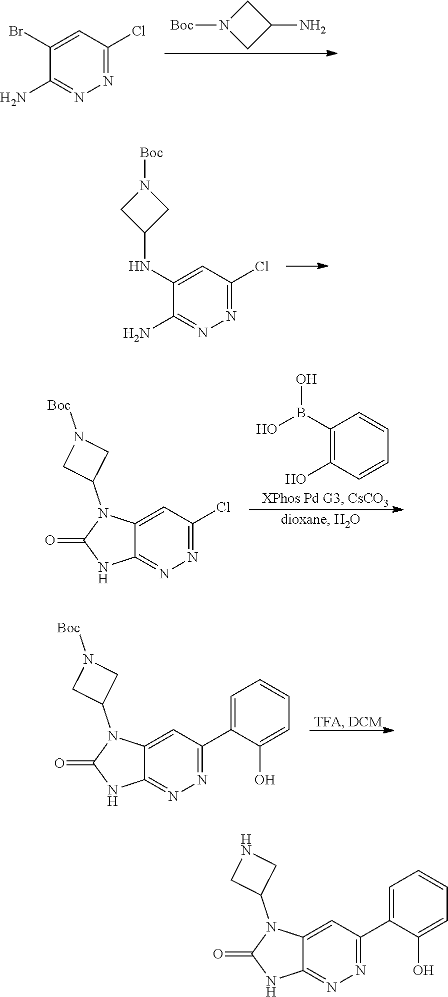

### Step 1: Preparation of tert-butyl 3-[(3-amino-6-chloropyridazin-4-yl)amino]azetidine-1-carboxylate

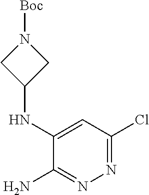 [SMILES: CC(C)(C)OC(=O)N1CC(Nc2cc(Cl)nnc2N)C1]

To a stirred solution of tert-butyl 3-aminoazetidine-1-carboxylate (1.65 g, 9.596 mmol, 2 equiv) and DIEA (6.20 g, 47.980 mmol, 10 equiv) in DMSO (16 ml) was added 4-bromo-6-chloropyridazin-3-amine (1 g, 4.798 mmol, 1.00 equiv). The resulting mixture was stirred at 120 degrees C. for 2 h. The reaction mixture was poured into 100 mL of water, the precipitated solid was collected by filtration and washed with H₂O, the resulting solid was dried under vacuum to afford tert-butyl 3-[(3-amino-6-chloropyridazin-4-yl)amino]azetidine-1-carboxylate (910 mg, 62.58%) as a white solid that was used in the next step directly without further purification. LCMS (ESI) m/z: [M+H]⁺=300.

### Step 2: Preparation of tert-butyl 3-{3-chloro-6-oxo-7H-imidazo[4,5-c]pyridazin-5-yl}azetidine-1-carboxylate

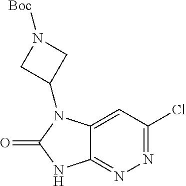 [SMILES: CC(C)(C)OC(=O)N1CC(n2c(=O)[nH]c3nnc(Cl)cc32)C1]

To a stirred solution of tert-butyl 3-[(3-amino-6-chloropyridazin-4-yl)amino]azetidine-1-carboxylate (500 mg, 1.668 mmol, 1.00 equiv) and TEA (675.14 mg, 6.672 mmol, 4 equiv) in THF (5 mL) was added triphosgene (989.95 mg, 3.336 mmol, 2 equiv). The resulting mixture was stirred at room temperature for 1.5 h. The reaction mixture was diluted with EtOAc (50 mL), washed with water (3×10 mL), and dried over anhydrous Na₂SO₄. After filtration, the filtrate was concentrated under reduced pressure. The residue was purified by silica gel column chromatography, eluted with CH₂Cl₂/MeOH (10:1) to afford tert-butyl 3-{3-chloro-6-oxo-7H-imidazo[4,5-c]pyridazin-5-yl}azetidine-1-carboxylate (300 mg, 55.21%) as a white solid. LCMS (ESI) m/z: [M+H]⁺=326.

### Step 3: Preparation of tert-butyl 3-[3-(2-hydroxyphenyl)-6-oxo-7H-imidazo[4,5-c]pyridazin-5-yl]azetidine-1-carboxylate

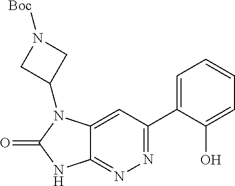 [SMILES: CC(C)(C)OC(=O)N1CC(n2c(=O)[nH]c3nnc(-c4ccccc4O)cc32)C1]

To a solution of tert-butyl 3-{3-chloro-6-oxo-7H-imidazo[4,5-c]pyridazin-5-yl}azetidine-1-carboxylate (250 mg, 0.767 mmol, 1.00 equiv), 2-hydroxyphenylboronic acid (317.57 mg, 2.301 mmol, 3 equiv) and Cs₂CO₃ (500.11 mg, 1.534 mmol, 2 equiv) in dioxane (1.5 ml) and H₂O (1.5 ml) was added BrettPhos Pd G3 (69.57 mg, 0.076 mmol, 0.1 equiv) under an atmosphere of dry nitrogen, the resulting mixture was stirred at 80 degree C. for 1.5 h under an atmosphere of dry nitrogen. The solid was filtered out, and the filtrate was concentrated under reduced pressure, the residue was purified by purified by flash C18-flash chromatography, elution gradient 0 to 80% MeCN in water (containing 0.1% NH₄HCO₃). Pure fractions were evaporated to dryness to afford tert-butyl 3-[3-(2-hydroxyphenyl)-6-oxo-7H-imidazo[4,5-c]pyridazin-5-yl]azetidine-1-carboxylate (48 mg, 16.31%) as a white solid. LCMS (ESI) m/z: [M+H]⁺=384

### Step 4: Preparation of 5-(azetidin-3-yl)-3-(2-hydroxyphenyl)-7H-imidazo[4,5-c]pyridazin-6-one

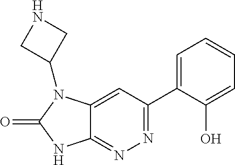 [SMILES: O=c1[nH]c2nnc(-c3ccccc3O)cc2n1C1CNC1]

A solution of tert-butyl 3-[3-(2-hydroxyphenyl)-6-oxo-7H-imidazo[4,5-c]pyridazin-5-yl]azetidine-1-carboxylate (45 mg, 0.117 mmol, 1.00 equiv) in TFA (1 ml) and DCM (2 ml) was stirred for 1 h at room temperature. The resulting mixture was concentrated under reduced pressure to afford intermediate 5 (20 mg, 60.15%) as a white solid that was used in the next step directly without further purification. LCMS (ESI) m/z: [M+H]⁺=284

### Preparation of 2-{6′,7′-dihydrospiro[azetidine-3,5′-pyrrolo[2,3-c]pyridazin]-3′-yl}phenol (I-118)

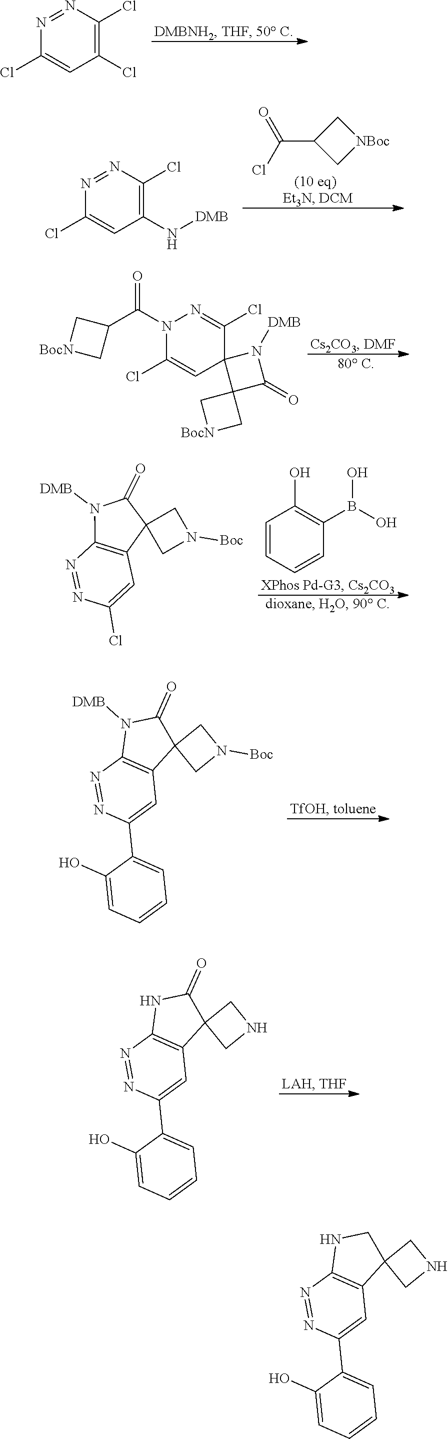

### Step 1: Preparation of 3,6-dichloro-N-[(3,4-dimethylphenyl)methyl]pyridazin-4-amine

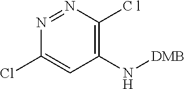

To a stirred solution of 3,4,6-trichloropyridazine (10 g, 54.520 mmol, 1.00 equiv) in THF (50 mL) was added (2,4-dimethoxyphenyl)methanamine (27.35 g, 163.560 mmol, 3.0 equiv) dropwise at room temperature under an atmosphere of dry nitrogen. The resulting mixture was stirred for 2 h at 50 degrees C. The resulting mixture was concentrated under reduced pressure. The residue was purified by silica gel column chromatography, eluted with PE/EA (1:1) to afford 3,6-dichloro-N-[(3,4-dimethylphenyl)methyl]pyridazin-4-amine (13 g, 84.50%) as a white solid. LCMS (ESI) m/z [M+H]⁺=578.

### Step 2: Preparation of tert-butyl 8-[1-(tert-butoxycarbonyl)azetidine-3-carbonyl]-6,9-dichloro-11-[(3,4-dimethylphenyl)methyl]-12-oxo-2,7,8,11-tetraazadispiro[3.0.5\hat{ }{5}0.2\hat{ }{4}]dodeca-6,9-diene-2-carboxylate

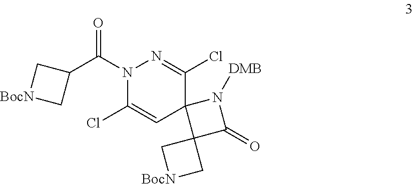

A solution of 3,6-dichloro-N-[(3,4-dimethylphenyl)methyl]pyridazin-4-amine (600 mg, 2.126 mmol, 1.00 equiv) and tert-butyl 3-(carboxy)azetidine-1-carboxylate (4.67 g, 21.260 mmol, 10 equiv), Et₃N (2.15 g, 21.260 mmol, 10 equiv) in DCM (10 mL) was stirred for 12 h at 25 degrees C. The resulting mixture was concentrated under reduced pressure. to afford tert-butyl 8-[1-(tert-butoxycarbonyl)azetidine-3-carbonyl]-6,9-dichloro-11-[(3,4-dimethylphenyl)methyl]-12-oxo-2,7,8,11-tetraazadispiro[3.0.5\hat{ }{5}0.2\hat{ }{4}]dodeca-6,9-diene-2-carboxylate (4.7 g, crude) as a light yellow solid. The crude product was used in the next step directly without further purification. LCMS (ESI) m/z [M+H]⁺=680.

### Step 3: Preparation of tert-butyl 3′-chloro-7′-[(3,4-dimethylphenyl)methyl]-6′-oxospiro[azetidine-3,5′-pyrrolo[2,3-c]pyridazine]-1-carboxylate

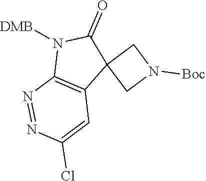

A solution of tert-butyl 8-[1-(tert-butoxycarbonyl)azetidine-3-carbonyl]-6,9-dichloro-11-[(3,4-dimethylphenyl)methyl]-12-oxo-2,7,8,11-tetraazadispiro[3.0.5\hat{ }{5}0.2\hat{ }{4}]dodeca-6,9-diene-2-carboxylate (4.5 g, 6.938 mmol, 1.00 equiv) and Cs₂CO₃ (1.81 g, 5.550 mmol, 0.80 equiv) in DMF (10 mL) was stirred for 12 h at 80 degrees C. under an atmosphere of dry nitrogen. The residue was purified by reverse phase flash chromatography with the following conditions: column, C18 silica gel; mobile phase, CH₃CN in water, 0% to 75% gradient in 50 min; detector, UV 254 nm. This resulted in tert-butyl 3′-chloro-7′-[(3,4-dimethylphenyl)methyl]-6′-oxospiro[azetidine-3,5′-pyrrolo[2,3-c]pyridazine]-1-carboxylate (800 mg, 26.88%) as a yellow solid. LCMS (ESI) m/z [M+H]⁺=461.

### Step 4: Preparation of tert-butyl 7′-[(3,4-dimethylphenyl)methyl]-3′-(2-hydroxyphenyl)-6′-oxospiro[azetidine-3,5′-pyrrolo[2,3-c]pyridazine]-1-carboxylate

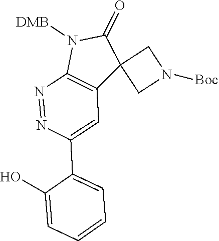

To a solution of tert-butyl 3′-chloro-7′-[(3,4-dimethylphenyl)methyl]-6′-oxospiro[azetidine-3,5′-pyrrolo[2,3-c]pyridazine]-1-carboxylate (600 mg, 1.399 mmol, 1 equiv) and 2-hydroxyphenylboronic acid (578.84 mg, 4.197 mmol, 3.0 equiv) in dioxane (5 mL) and H₂O (1 mL) were added Cs₂CO₃ (1367.33 mg, 4.197 mmol, 3.0 equiv) and XPhos Pd G3 (236.82 mg, 0.280 mmol, 0.2 equiv). After stirring for 4 h at 80° C. under a nitrogen atmosphere, the resulting mixture was concentrated under reduced pressure. The residue was purified by silica gel column chromatography, eluted with PE/EA (1:1) to afford tert-butyl 7′-[(3,4-dimethylphenyl)methyl]-3′-(2-hydroxyphenyl)-6′-oxospiro[azetidine-3,5′-pyrrolo[2,3-c]pyridazine]-1-carboxylate (400 mg, 58.77%) as a light yellow solid. LCMS (ESI) m/z [M+H]⁺=519.

### Step 5: Preparation of 3′-(2-hydroxyphenyl)-7′H-spiro[azetidine-3,5′-pyrrolo[2,3-c]pyridazin]-6′-one

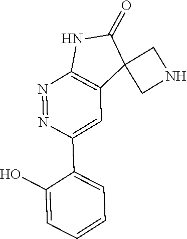 [SMILES: O=C1Nc2nnc(-c3ccccc3O)cc2C12CNC2]

A solution of tert-butyl 7′-[(3,4-dimethylphenyl)methyl]-3′-(2-hydroxyphenyl)-6′-oxospiro[azetidine-3,5′-pyrrolo[2,3-c]pyridazine]-1-carboxylate (400 mg, 0.822 mmol, 1 equiv) and CF₃SO₃H (2 mL) in toluene (2 mL) was stirred for 4 h at 120° C. under an atmosphere of dry nitrogen. The mixture was acidified to pH 6 with saturated NaHCO₃ (aq.). The resulting mixture was concentrated under reduced pressure. The residue was purified by reverse phase flash chromatography with the following conditions: column, silica gel; mobile phase, MeCN in water, 0% to 100% gradient in 50 min; detector, UV 254 nm. This resulted in 3′-(2-hydroxyphenyl)-7′H-spiro[azetidine-3,5′-pyrrolo[2,3-c]pyridazin]-6′-one (200 mg, 90.68%) as a light yellow solid. LCMS (ESI) m/z [M+H]⁺=269.

### Step 6: Preparation of 2-{6′,7′-dihydrospiro[azetidine-3,5′-pyrrolo[2,3-c]pyridazin]-3′-yl}phenol

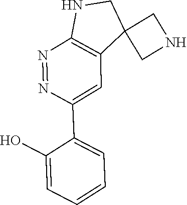 [SMILES: Oc1ccccc1-c1cc2c(nn1)NCC21CNC1]

To a stirred solution of 3′-(2-hydroxyphenyl)-7′H-spiro[azetidine-3,5′-pyrrolo[2,3-c]pyridazin]-6′-one (100 mg, 0.373 mmol, 1.00 equiv) in THF (10 mL, 123.430 mmol, 331.13 equiv) were added LAH (42.44 mg, 1.119 mmol, 3.0 equiv) dropwise at 0 degrees C. under an atmosphere of dry nitrogen. The reaction was quenched with sat. NH₄Cl (aq.) at 0 degrees C. The resulting mixture was extracted with EtOAc (3×20 mL). The combined organic layers were dried over anhydrous Na₂SO₄. After filtration, the filtrate was concentrated under reduced pressure. The residue was purified by reverse phase flash chromatography with the following conditions: column, C18 silica gel; mobile phase, MeOH in water, 0% to 50% gradient in 25 min; detector, UV 254 nm. This resulted in 2-{6′,7′-dihydrospiro[azetidine-3,5′-pyrrolo[2,3-c]pyridazin]-3′-yl}phenol (18 mg, 18.99%) as a yellow solid. LCMS (ESI) m/z [M+H]⁺=255.

### Preparation of 2-[5-(azetidin-3-yl)-5-methyl-6H,7H-pyrrolo[2,3-c]pyridazin-3-yl]phenol (I-119)

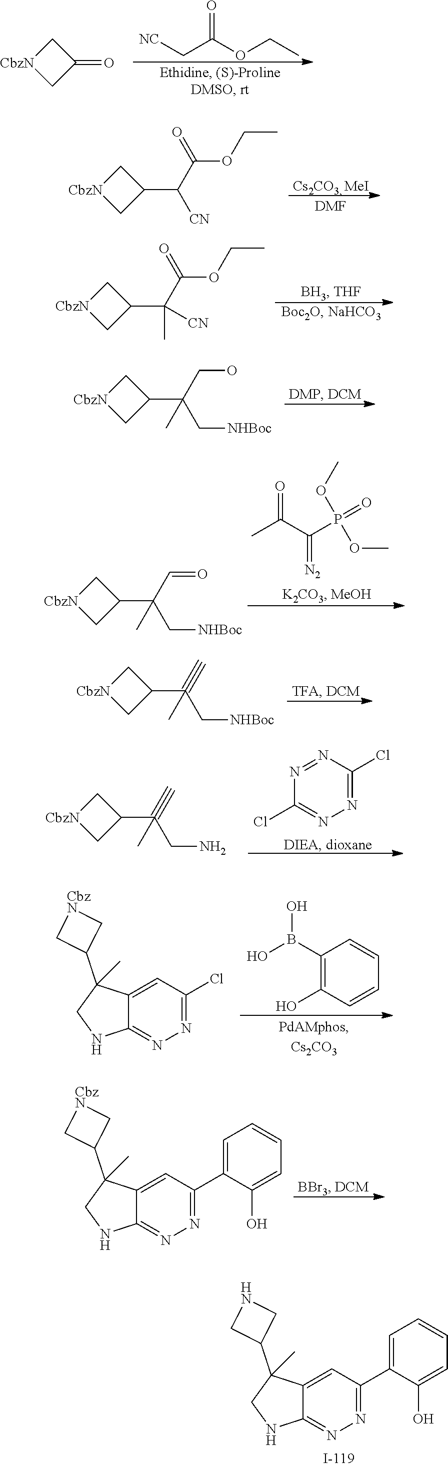

### Step 1: Preparation of benzyl 3-(1-cyano-2-ethoxy-2-oxoethyl)azetidine-1-carboxylate

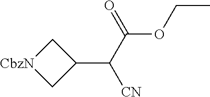 [SMILES: CCOC(=O)C(C#N)C1CN(C(=O)OCc2ccccc2)C1]

To a stirred mixture of benzyl 3-oxoazetidine-1-carboxylate (5.0 g, 24.365 mmol, 1.00 equiv) and ethyl cyanoacetate (4.13 g, 36.547 mmol, 1.5 equiv) in DMSO (10 mL) was added diethyl 2,6-dimethyl-1,4-dihydropyridine-3,5-dicarboxylate (12.34 g, 48.730 mmol, 2 equiv) and (2S)-pyrrolidine-2-carboxylic acid (1.12 g, 9.746 mmol, 0.4 equiv) at room temperature. The resulting mixture was stirred for 1 days at room temperature. The desired product could be detected by LCMS. The resulting mixture was extracted with EtOAc (2×50 mL). The combined organic layers were washed with brine (2×20 mL), and dried over anhydrous Na₂SO₄. After filtration, the filtrate was concentrated under reduced pressure. the residue was purified by reverse phase flash chromatography with the following conditions: column, silica gel; mobile phase, MeCN in water, 0% to 100% gradient in 25 min; detector, UV 254 nm. This resulted in benzyl 3-(1-cyano-2-ethoxy-2-oxoethyl)azetidine-1-carboxylate (3.1 g, 42.08%) as a light yellow solid. LCMS (ESI) m/z [M+H]⁺=303.

### Step 2: Preparation of benzyl 3-(1-cyano-2-ethoxy-1-methyl-2-oxoethyl)azetidine-1-carboxylate

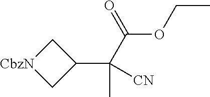 [SMILES: CCOC(=O)C(C)(C#N)C1CN(C(=O)OCc2ccccc2)C1]

To a stirred mixture of benzyl 3-(1-cyano-2-ethoxy-2-oxoethyl)azetidine-1-carboxylate (3 g, 9.923 mmol, 1.00 equiv) and Cs₂CO₃ (9.70 g, 29.769 mmol, 3 equiv) in DMF (5 mL) was added Mel (1.41 g, 9.923 mmol, 1 equiv) at room temperature. The resulting mixture was stirred overnight at room temperature. The desired product could be detected by LCMS. The residue was purified by reverse phase flash chromatography with the following conditions: column, silica gel; mobile phase, MeCN in water, 0% to 100% gradient in 25 min; detector 41%, UV 254 nm. The resulting mixture was concentrated under reduced pressure. This resulted in benzyl 3-(1-cyano-2-ethoxy-1-methyl-2-oxoethyl)azetidine-1-carboxylate (950 mg, 30.26%) as a brown yellow solid. LCMS (ESI) m/z [M+H]⁺=317.

### Step 3: Preparation of benzyl 3-{1-[(tert-butoxycarbonyl)amino]-3-hydroxy-2-methylpropan-2-yl}azetidine-1-carboxylate

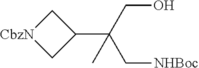 [SMILES: CC(C)(C)OC(=O)NCC(C)(CO)C1CN(C(=O)OCc2ccccc2)C1]

To a stirred solution of benzyl 3-(1-cyano-2-ethoxy-1-methyl-2-oxoethyl)azetidine-1-carboxylate (1.3 g, 4.109 mmol, 1 equiv) in THF (30 mL) were added BH₃-THF (0.71 g, 8.218 mmol, 2.0 equiv) dropwise at 0° C. under an atmosphere of dry nitrogen. The reaction was quenched with MeOH at 0° C. The mixture was acidified to pH 13 with saturated NaHCO₃ (aq.). To the above mixture was added Boc₂O (3.59 g, 16.436 mmol, 4.0 equiv) dropwise at room temperature. The resulting mixture was stirred for additional 1 h at room temperature. The resulting mixture was diluted with water (50 mL). The resulting mixture was extracted with EtOAc (3×100 mL). The combined organic layers were dried over anhydrous Na₂SO₄. After filtration, the filtrate was concentrated under reduced pressure. The crude product was used in the next step directly without further purification. This resulted in benzyl 3-{1-[(tert-butoxycarbonyl)amino]-3-hydroxy-2-methylpropan-2-yl}azetidine-1-carboxylate (2.1 g, crude) as a yellow oil. LCMS (ESI) m/z [M+H]⁺=379.

### Step 4: Preparation of benzyl 3-{1-[(tert-butoxycarbonyl)amino]-2-methyl-3-oxopropan-2-yl}azetidine-1-carboxylate

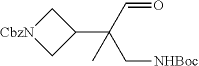 [SMILES: CC(C)(C)OC(=O)NCC(C)(C=O)C1CN(C(=O)OCc2ccccc2)C1]

A solution of benzyl 3-{1-[(tert-butoxycarbonyl)amino]-3-hydroxy-2-methylpropan-2-yl}azetidine-1-carboxylate (2.0 g, 5.284 mmol, 1 equiv) and DMP (13.45 g, 31.704 mmol, 6.0 equiv) in DCM (20 mL) was stirred for 2 h at room temperature. The reaction was quenched with sat. Na₂S₂O₃ (aq.) at room temperature. The resulting mixture was diluted with water (200 mL). The resulting mixture was extracted with CH₂Cl₂ (3×100 mL). The combined organic layers were dried over anhydrous Na₂SO₄. After filtration, the filtrate was concentrated under reduced pressure. This resulted in benzyl 3-{1-[(tert-butoxycarbonyl)amino]-2-methyl-3-oxopropan-2-yl}azetidine-1-carboxylate (1.9 g, 95.51%) as a yellow oil. The crude product was used in the next step directly without further purification. LCMS (ESI) m/z [M+H]⁺=377.

### Step 5: Preparation of benzyl 3-(2-{[(tert-butoxycarbonyl)amino]methyl}but-3-yn-2-yl)azetidine-1-carboxylate

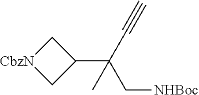 [SMILES: C#CC(C)(CNC(=O)OC(C)(C)C)C1CN(C(=O)OCc2ccccc2)C1]

A solution of benzyl 3-{1-[(tert-butoxycarbonyl)amino]-2-methyl-3-oxopropan-2-yl}azetidine-1-carboxylate (1.9 g, 5.047 mmol, 1 equiv) in MeOH (10 mL) was treated with K₂CO₃ (2.09 g, 15.141 mmol, 3.0 equiv) for 10 min at 0° C. under an atmosphere of dry nitrogen followed by the addition of dimethyl (diazomethyl)phosphonate (969.61 mg, 5.047 mmol, 1.0 equiv) dropwise at 0° C. The resulting mixture was stirred for 4 h at room temperature. The resulting mixture was diluted with water (100 mL). The resulting mixture was extracted with EtOAc (3×150 mL). The combined organic layers were dried over anhydrous Na₂SO₄. After filtration, the filtrate was concentrated under reduced pressure. The residue was purified by reverse phase flash chromatography with the following conditions: column, silica gel; mobile phase, MeCN in water, 0% to 100% gradient in 50 min; detector, UV 254 nm. This resulted in benzyl benzyl 3-(2-{[(tert-butoxycarbonyl)amino]methyl}but-3-yn-2-yl)azetidine-1-carboxylate (500 mg, 26.60%) as a yellow oil. LCMS (ESI) m/z [M+H]⁺=373.

### Step 6: Preparation of benzyl 3-(1-amino-2-methylbut-3-yn-2-yl)azetidine-1-carboxylate

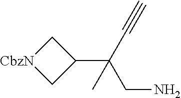 [SMILES: C#CC(C)(CN)C1CN(C(=O)OCc2ccccc2)C1]

A solution of benzyl 3-(2-{[(tert-butoxycarbonyl)amino]methyl}but-3-yn-2-yl)azetidine-1-carboxylate (500 mg, 1.342 mmol, 1 equiv) and TFA (1 mL) in DCM (4 mL) was stirred for 2 h at room temperature. The resulting mixture was concentrated under reduced pressure. The crude product was used in the next step directly without further purification. This resulted in benzyl 3-(1-amino-2-methylbut-3-yn-2-yl)azetidine-1-carboxylate (500 mg, 136.76%) as a yellow oil. LCMS (ESI) m/z [M+H]⁺=273.

### Step 7: Preparation of benzyl 3-{3-chloro-5-methyl-6H,7H-pyrrolo[2,3-c]pyridazin-5-yl}azetidine-1-carboxylate

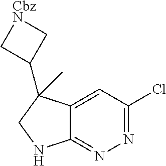 [SMILES: CC1(C2CN(C(=O)OCc3ccccc3)C2)CNc2nnc(Cl)cc21]

A solution of benzyl 3-(1-amino-2-methylbut-3-yn-2-yl)azetidine-1-carboxylate (500 mg, 1.836 mmol, 1.00 equiv), DIEA (1423.66 mg, 11.016 mmol, 6.0 equiv) and dichloro-1,2,4,5-tetrazine (554.25 mg, 3.672 mmol, 2.0 equiv) in dioxane (10 mL) was stirred for 12 h at 100 degrees C. under an atmosphere of dry nitrogen. The resulting mixture was concentrated under reduced pressure. The residue was purified by reverse phase flash chromatography with the following conditions: column, silica gel; mobile phase, MeCN in water, 0% to 50% gradient in 40 min; detector, UV 254 nm. This resulted in benzyl 3-{3-chloro-5-methyl-6H,7H-pyrrolo[2,3-c]pyridazin-5-yl}azetidine-1-carboxylate (250 mg, 37.95%) as a Brown yellow oil. LCMS (ESI) m/z [M+H]⁺=359.

### Step 8: Preparation of benzyl 3-[3-(2-hydroxyphenyl)-5-methyl-6H,7H-pyrrolo[2,3-c]pyridazin-5-yl]azetidine-1-carboxylate

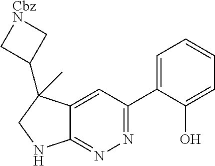 [SMILES: CC1(C2CN(C(=O)OCc3ccccc3)C2)CNc2nnc(-c3ccccc3O)cc21]

To a solution of benzyl 3-{3-chloro-5-methyl-6H,7H-pyrrolo[2,3-c]pyridazin-5-yl}azetidine-1-carboxylate (250 mg, 0.697 mmol, 1.00 equiv) and 2-hydroxyphenylboronic acid (288.29 mg, 2.091 mmol, 3.0 equiv) in Dioxane (4 mL) and H₂O (0.8 mL) were added Cs₂CO₃ (681.00 mg, 2.091 mmol, 3.0 equiv) and Pd(AMPHOS)₂Cl₂ (98.66 mg, 0.139 mmol, 0.2 equiv). After stirring for 2 h at 80 degrees C. under a nitrogen atmosphere, the resulting mixture was concentrated under reduced pressure. The resulting mixture was diluted with water (10 ml). The resulting mixture was extracted with EtOAc (3×10 mL). The combined organic layers were dried over anhydrous Na₂SO₄. After filtration, the filtrate was concentrated under reduced pressure. The residue was purified by reverse phase flash chromatography with the following conditions: column, silica gel; mobile phase, MeCN in water, 0% to 100% gradient in 50 min; detector, UV 254 nm. This resulted in benzyl 3-[3-(2-hydroxyphenyl)-5-methyl-6H,7H-pyrrolo[2,3-c]pyridazin-5-yl]azetidine-1-carboxylate (200 mg, 68.93%) as a yellow oil. LCMS (ESI) m/z [M+H]⁺=417.

### Step 9: Preparation of 2-[5-(azetidin-3-yl)-5-methyl-6H,7H-pyrrolo[2,3-c]pyridazin-3-yl]phenol (I-119)

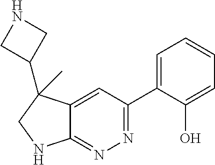 [SMILES: CC1(C2CNC2)CNc2nnc(-c3ccccc3O)cc21]

To a stirred solution of benzyl 3-[3-(2-hydroxyphenyl)-5-methyl-6H,7H-pyrrolo[2,3-c]pyridazin-5-yl]azetidine-1-carboxylate (60 mg, 0.144 mmol, 1.00 equiv) in DCM (3 mL) were added BBr₃ (360.91 mg, 1.440 mmol, 10 equiv) dropwise at 0 degrees C. The crude product was purified by Prep-HPLC with the following conditions (Column: XBridge Prep C18 OBD Column, 19*150 mm, 5 μm; Mobile Phase A: water (10 mmol/L NH₄HCO₃), Mobile Phase B: CH₃CN; Flow rate: 25 mL/min; Gradient: 12% B to 28% B in 6 min, 28% B; Wave Length: 254/220 nm; to afford I-119 (12.9 mg, 30.26%) as a yellow solid. ¹H NMR (300 MHz, Methanol-d₄) δ 8.09 (s, 1H), 7.94 (dd, J=8.3, 1.6 Hz, 1H), 7.36 (ddd, J=8.7, 7.2, 1.6 Hz, 1H), 7.09-6.97 (m, 2H), 3.97-3.80 (m, 3H), 3.78-3.70 (m, 2H), 3.56 (d, J=10.0 Hz, 1H), 3.52-3.40 (m, 1H), 1.52 (s, 3H). LCMS (ESI) m/z [M+H]⁺=282.15.

### Preparation of 2-(6-(azetidin-3-yl)-6-methyl-6,7-dihydro-5H-pyrrolo[2,3-c]pyridazin-3-yl)phenol (I-120)

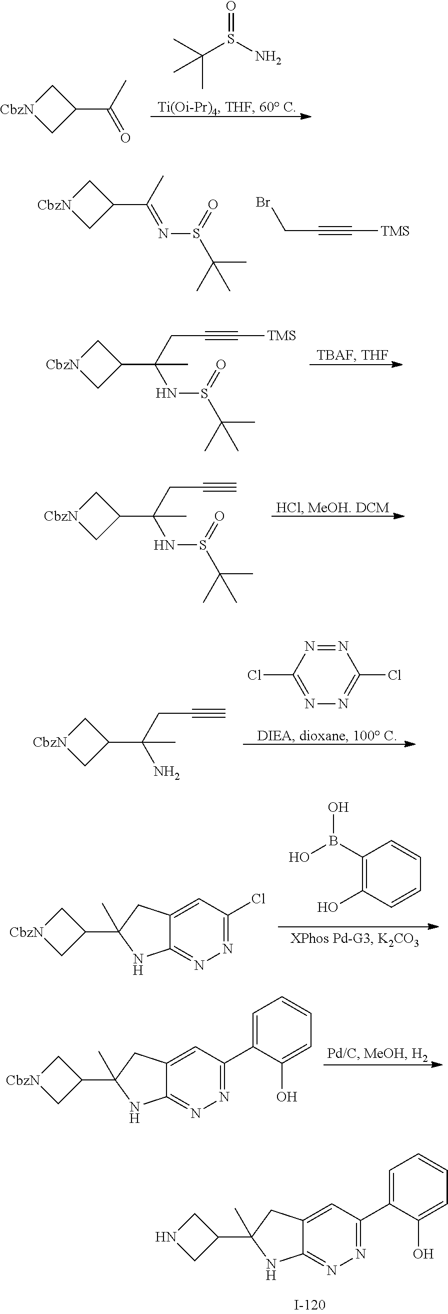

### Step 1: Preparation of benzyl (E)-3-(1-((tert-butylsulfinyl)imino)ethyl)azetidine-1-carboxylate

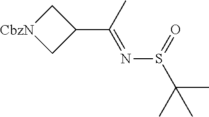 [SMILES: C/C(=N\S(=O)C(C)(C)C)C1CN(C(=O)OCc2ccccc2)C1]

To a stirred mixture of tert-butanesulfinamide (1.42 g, 11.716 mmol, 1.00 equiv) and benzyl 3-acetylazetidine-1-carboxylate (3.01 g, 12.888 mmol, 1.1 equiv) in THF were added Ti(Oi-Pr)₄ (6.66 g, 23.432 mmol, 2.0 equiv) in portions at room temperature under an atmosphere of dry nitrogen. The resulting mixture was stirred for 5 h at 60 degrees C. under an atmosphere of dry nitrogen. The mixture was allowed to cool down to room temperature. The reaction was quenched by the addition of water (20 mL) at room temperature. The precipitated solids were collected by filtration and washed with ethyl acetate (3×100 mL). The resulting mixture was extracted with EtOAc (300 mL). The combined organic layers were washed with deionized water (3×100 mL), and dried over anhydrous Na₂SO₄. After filtration, the filtrate was concentrated under reduced pressure. This resulted in benzyl (E)-3-(1-((tert-butylsulfinyl)imino)ethyl)azetidine-1-carboxylate (3.71 g, 94.12%) as a light yellow oil. LCMS (ESI) m/z: [M+H]⁺=337.

### Step 2: Preparation of benzyl 3-(2-((tert-butylsulfinyl)amino)-5-(trimethylsilyl)pent-4-yn-2-yl)azetidine-1-carboxylate

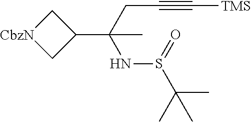 [SMILES: CC(CC#C[Si](C)(C)C)(NS(=O)C(C)(C)C)C1CN(C(=O)OCc2ccccc2)C1]

To a stirred mixture of (3-bromoprop-1-yn-1-yl)trimethylsilane (1.77 g, 9.276 mmol, 6 equiv) and Zn (606.56 mg, 9.276 mmol, 6 equiv) in THF were added intermediate 2 (520 mg, 1.546 mmol, 1.00 equiv) dropwise at room temperature under an atmosphere of dry nitrogen. The resulting mixture was stirred for additional 16 h at 50 degrees C. The mixture was allowed to cool down to room temperature. The resulting mixture was diluted with water (100 mL) and extracted with EtOAc (3×100 mL). The combined organic layers were washed with brine (100 mL), and dried over anhydrous Na₂SO₄. After filtration, the filtrate was concentrated under reduced pressure. The residue was purified by reverse phase flash chromatography with the following conditions: column, C18 silica gel; mobile phase, MeOH in water, 10% to 50% gradient in 10 min; detector, UV 254 nm. This resulted in benzyl 3-(2-((tert-butylsulfinyl)amino)-5-(trimethylsilyl)pent-4-yn-2-yl)azetidine-1-carboxylate (554.7 mg, 80.1%) as a light yellow oil. LCMS (ESI) m/z: [M+H]⁺=449.

### Step 3: Preparation of benzyl 3-(2-((tert-butylsulfinyl)amino)pent-4-yn-2-yl)azetidine-1-carboxylate

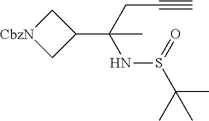 [SMILES: C#CCC(C)(NS(=O)C(C)(C)C)C1CN(C(=O)OCc2ccccc2)C1]

A mixture of benzyl 3-(2-((tert-butylsulfinyl)amino)-5-(trimethylsilyl)pent-4-yn-2-yl)azetidine-1-carboxylate (1.0 g, 2.23 mmol, 1.00 equiv) and TBAF (2.91 g, 11.15 mmol, 5 equiv) in THF (50 mL) was stirred for 2 h at room temperature. The reaction mixture was concentrated under reduced pressure. The residue was purified by silica gel column chromatography, eluted with PE/EA (2:1) to afford benzyl 3-(2-((tert-butylsulfinyl)amino)pent-4-yn-2-yl)azetidine-1-carboxylate (755.4 mg, 90.0%) as yellow oil. LCMS (ESI) m/z: [M+H]⁺=377.

### Step 4: Preparation of benzyl 3-(2-aminopent-4-yn-2-yl)azetidine-1-carboxylate

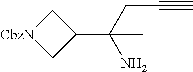 [SMILES: C#CCC(C)(N)C1CN(C(=O)OCc2ccccc2)C1]

To a stirred mixture of benzyl 3-(2-((tert-butylsulfinyl)amino)pent-4-yn-2-yl)azetidine-1-carboxylate (930.0 mg, 2.47 mmol, 1.00 equiv) in DCM were added HCl (0.75 mL) and MeOH (10.00 mL) in portions at room temperature. The resulting mixture was stirred for 1 h at room temperature. The mixture was neutralized to pH 7 with saturated NaHCO₃ (aq.). The resulting mixture was extracted with EtOAc (3×30 mL). The combined organic layers were washed with brine (1×40 mL), and dried over anhydrous Na₂SO₄. After filtration, the filtrate was concentrated under reduced pressure. The residue was purified by reverse phase flash chromatography with the following conditions:column, C18 silica gel; mobile phase, MeOH in water, 10% to 50% gradient in 10 min; detector, UV 254 nm, to afford benzyl 3-(2-aminopent-4-yn-2-yl)azetidine-1-carboxylate (490 mg, 73.5%) as a yellow solid. LCMS (ESI) m/z: [M+H]⁺=273.

### Step 5: Preparation of benzyl 3-(3-chloro-6-methyl-6,7-dihydro-5H-pyrrolo[2,3-c]pyridazin-6-yl)azetidine-1-carboxylate

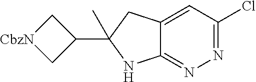 [SMILES: CC1(C2CN(C(=O)OCc3ccccc3)C2)Cc2cc(Cl)nnc2N1]

To a stirred solution of benzyl 3-(2-aminopent-4-yn-2-yl)azetidine-1-carboxylate (490 mg, 1.799 mmol, 1.00 equiv) and dichloro-1,2,4,5-tetrazine (543.17 mg, 3.598 mmol, 2 equiv) in dioxane (20 mL) was added DIEA (697.59 mg, 5.397 mmol, 3 equiv) dropwise at room temperature under an atmosphere of dry nitrogen. The resulting mixture was stirred for 3 h at 100 degrees C. under an atmosphere of dry nitrogen. The mixture was allowed to cool down to room temperature. The resulting mixture was filtered, the filter cake was washed with EtOAc (3×40 mL). The filtrate was concentrated under reduced pressure. The residue was purified by reverse phase flash with the following conditions (column, C18 silica gel; mobile phase, MeOH in water, 10% to 50% gradient in 10 min; detector, UV 254 nm) to afford benzyl 3-(3-chloro-6-methyl-6,7-dihydro-5H-pyrrolo[2,3-c]pyridazin-6-yl)azetidine-1-carboxylate (500 mg, 77.45%) as a yellow solid. LCMS (ESI) m/z: [M+H]⁺=359.

### Step 6: Preparation of benzyl 3-(3-(2-hydroxyphenyl)-6-methyl-6,7-dihydro-5H-pyrrolo[2,3-c]pyridazin-6-yl)azetidine-1-carboxylate

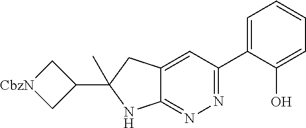 [SMILES: CC1(C2CN(C(=O)OCc3ccccc3)C2)Cc2cc(-c3ccccc3O)nnc2N1]

A mixture of benzyl 3-(3-chloro-6-methyl-6,7-dihydro-5H-pyrrolo[2,3-c]pyridazin-6-yl)azetidine-1-carboxylate (200 mg, 0.557 mmol, 1.00 equiv), XPhos Pd G3 (47.18 mg, 0.056 mmol, 0.1 equiv) and Cs₂CO₃ (544.80 mg, 1.671 mmol, 3.0 equiv) in Dioxane (4 mL) and H₂O (0.5 mL) was stirred for 4 h at 80 degrees C. under an atmosphere of dry nitrogen. The mixture was allowed to cool down to room temperature. The resulting mixture was diluted with water (50 mL) and extracted with EtOAc (3×50 mL). The combined organic layers were washed with brine (1×50 mL), and dried over anhydrous Na₂SO₄. After filtration, the filtrate was concentrated under reduced pressure. The residue product was purified by reverse phase flash with the following conditions (column, C18 silica gel; mobile phase, MeOH in water, 10% to 50% gradient in 25 min; detector, UV 254 nm to afford benzyl 3-(3-(2-hydroxyphenyl)-6-methyl-6,7-dihydro-5H-pyrrolo[2,3-c]pyridazin-6-yl)azetidine-1-carboxylate (100 mg, 43.08%) as a yellow solid. LCMS (ESI) m/z: [M+H]⁺=417.

### Step 7: Preparation of 2-(6-(azetidin-3-yl)-6-methyl-6,7-dihydro-5H-pyrrolo[2,3-c]pyridazin-3-yl)phenol (I-120)

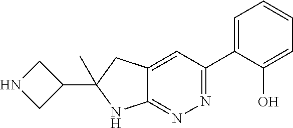 [SMILES: CC1(C2CNC2)Cc2cc(-c3ccccc3O)nnc2N1]

To a solution of benzyl 3-(3-(2-hydroxyphenyl)-6-methyl-6,7-dihydro-5H-pyrrolo[2,3-c]pyridazin-6-yl)azetidine-1-carboxylate (20 mg, 0.048 mmol, 1 equiv) in MeOH (1 mL) was added Pd(OH)₂/C (10%, 10 mg) under an atmosphere of dry nitrogen in a 25 mL round-bottom flask. The mixture was hydrogenated at room temperature for 3 h under hydrogen atmosphere using a hydrogen balloon, filtered through a Celite pad and concentrated under reduced pressure. The crude product (21 mg) was purified by Prep-HPLC with the following conditions (Column: XBridge Prep C18 OBD Column, 19*150 mm, 5 μm; Mobile Phase A: Water (10 mmol/L NH₄HCO₃), Mobile Phase B: CH₃CN; Flow rate: 25 mL/min; Gradient: 5% B to 40% B in 7 min, 40% B; Wave Length: 254/220 nm; RT1(min): 5.85; Number Of Runs: 0) to afford I-120 (6 mg, 44.25%) as a white solid. ¹H NMR (300 MHz, Methanol-d4) δ 7.93 (d, J=7.8 Hz, 1H), 7.76 (d, J=8.1 Hz, 1H), 7.48-7.09 (m, 1H), 7.09-6.70 (m, 2H), 4.01-3.56 (m, 4H), 3.26-2.94 (m, 3H), 1.34 (d, J=10.3 Hz, 3H). LCMS (ESI) m/z: [M−H]⁻=283.25.

### Preparation of (2S,4R)-4-hydroxy-1-[(2R)-2-{3-[4-(2-{3-[3-(2-hydroxyphenyl)-7H-pyrrolo[2,3-c]pyridazin-6-yl]azetidin-1-yl}ethyl)piperazin-1-yl]-1,2-oxazol-5-yl}-3-methylbutanoyl]-N-[(1S)-1-[4-(4-methyl-1,3-thiazol-5-yl)phenyl]ethyl]pyrrolidine-2-carboxamide (Compound 273)

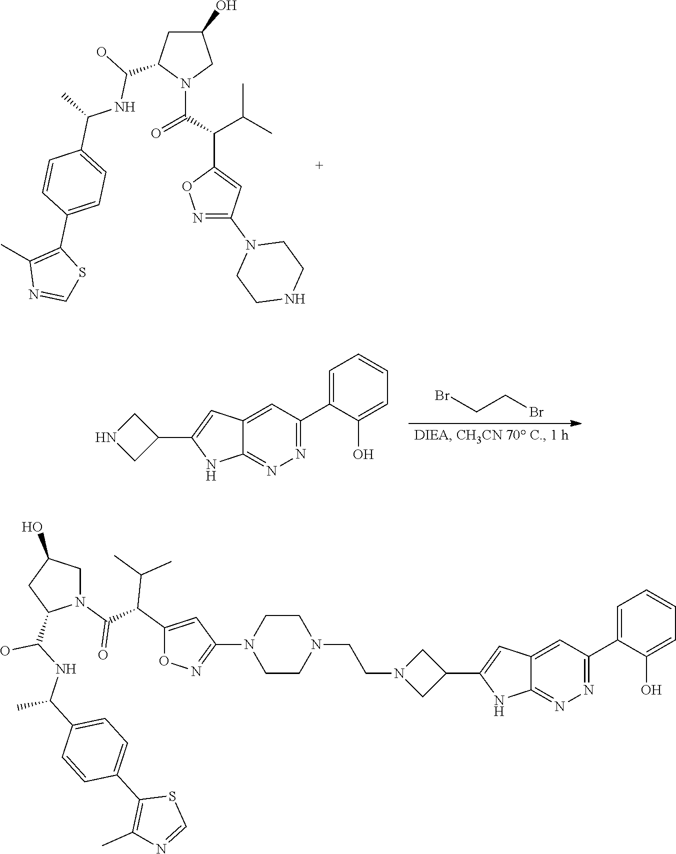

To a stirred solution of (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (30.00 mg, 0.053 mmol, 1.00 equiv) and 2-[6-(azetidin-3-yl)-7H-pyrrolo[2,3-c]pyridazin-3-yl]phenol (42.29 mg, 0.159 mmol, 3.00 equiv) in CH₃CN (1.00 mL) was added 1,2-dibromoethane (9.94 mg, 0.053 mmol, 1.00 equiv) and DIEA (41.05 mg, 0.318 mmol, 6.00 equiv) at room temperature. The resulting mixture was stirred for 2 h at 70 degrees C. The mixture was allowed to cool down to room temperature. The crude product was purified by Prep-HPLC with the following conditions: Column, XBridge Shield RP18 OBD Column, 19*150 mm, 5 μm; mobile phase, Water (10 mmol/L NH₄HCO₃) and MeOH (60% MeOH up to 77% in 8 min); Detector, UV 254/220 nm. This resulted in title compound (2.1 mg, 4.39%) as a yellow solid. ¹H NMR (400 MHz, DMSO-d⁶) δ 14.16 (s, 1H), 12.76-12.19 (m, 1H), 8.98 (s, 1H), 8.55 (s, 1H), 8.39 (d, J=7.7 Hz, 1H), 8.03 (d, J=7.7 Hz, 1H), 7.49-7.41 (m, 2H), 7.37 (d, J=8.2 Hz, 2H), 7.33-7.26 (m, 1H), 6.95 (t, J=8.2 Hz, 2H), 6.54 (d, J=12.9 Hz, 1H), 6.14 (s, 1H), 5.10 (d, J=3.7 Hz, 1H), 5.01-4.86 (m, 1H), 4.36 (t, J=7.8 Hz, 1H), 4.31-4.24 (m, 1H), 3.91-3.81 (m, 1H), 3.75-3.64 (m, 3H), 3.57 (d, J=10.0 Hz, 1H), 3.51 (s, 1H), 3.46-3.39 (m, 1H), 3.30-3.24 (m, 2H), 3.20-3.12 (m, 4H), 2.63 (t, J=7.0 Hz, 2H), 2.46 (s, 3H), 2.37-2.33 (m, 1H), 2.27-2.11 (m, 1H), 2.06-1.96 (m, 1H), 1.84-1.72 (m, 1H), 1.38 (d, J=7.0 Hz, 3H), 0.96 (t, J=6.6 Hz, 3H), 0.81 (d, J=6.6 Hz, 3H). LCMS (ESI) m/z: [M+H]⁺=859.50.

### Preparation of (2S,4R)-4-hydroxy-1-[(2R)-2-[3-(2-{4-[3-(2-hydroxyphenyl)-5-methyl-7H-pyrrolo[2,3-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 270)

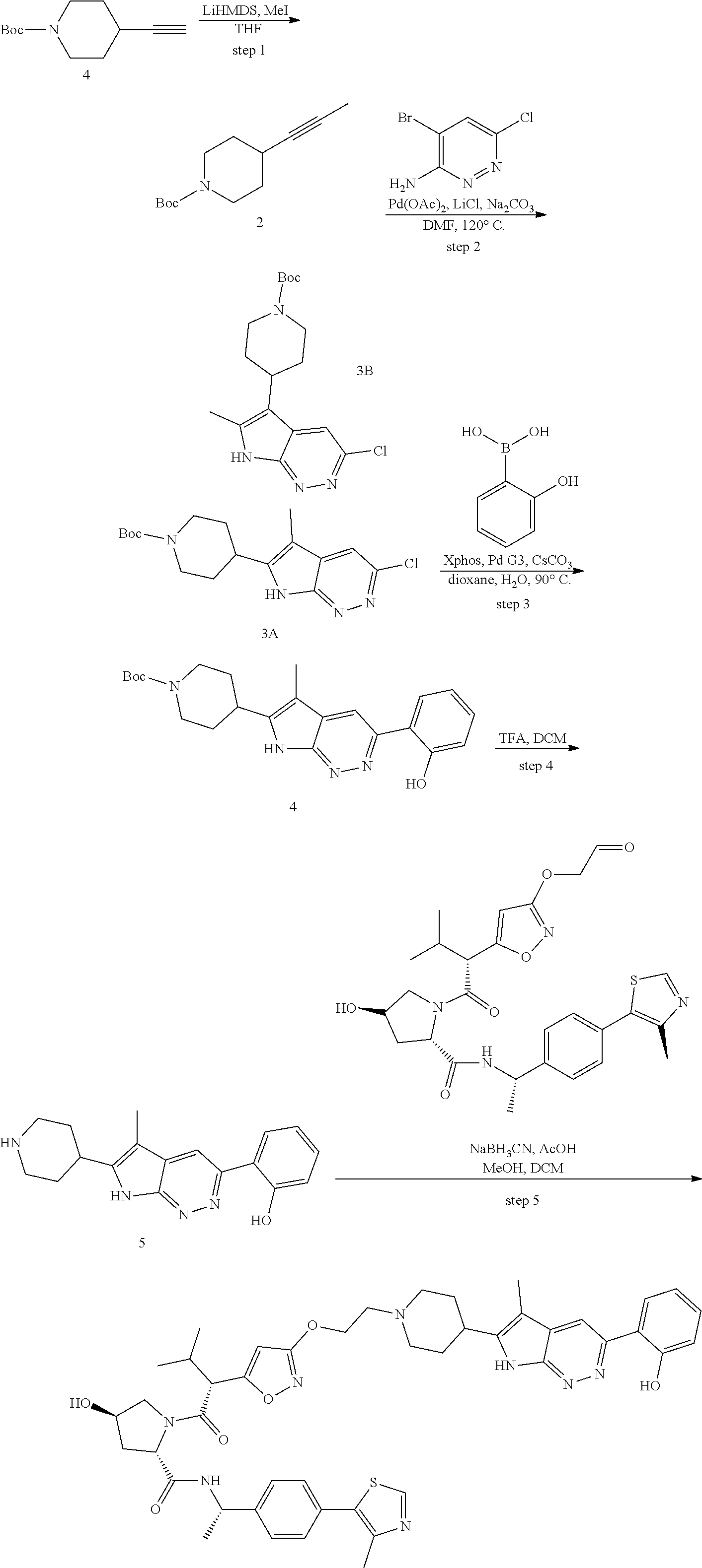

### Step 1: Preparation of tert-butyl 4-(prop-1-yn-1-yl)piperidine-1-carboxylate (Intermediate 2)

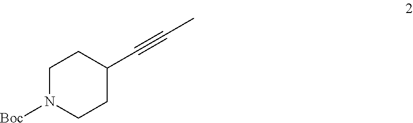 [SMILES: CC#CC1CCN(C(=O)OC(C)(C)C)CC1]

To a stirred solution of tert-butyl 4-ethynylpiperidine-1-carboxylate (2 g, 9.556 mmol, 1.00 equiv) in THF (30 mL) was added LiHMDS (4.80 g, 28.668 mmol, 3.00 equiv) dropwise at −78° C. under an atmosphere of dry nitrogen. The resulting mixture was stirred for 1 h at −78° C. under an atmosphere of dry nitrogen. To the above mixture was added Mel (6.78 g, 47.780 mmol, 5 equiv) dropwise at −78° C. The resulting mixture was stirred overnight at room temperature. The residue was purified by silica gel column chromatography, eluted with PE/EA (9:1) to afford Intermediate 2 (1.3 g, 60.92%) as a yellow solid. LCMS (ESI) m/z: [M+H]⁺=224.

### Step 2: Preparation of tert-butyl 4-[3-chloro-5-methyl-7H-pyrrolo[2,3-c]pyridazin-6-yl]piperidine-1-carboxylate (Intermediate 3A)

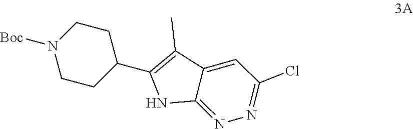 [SMILES: Cc1c(C2CCN(C(=O)OC(C)(C)C)CC2)[nH]c2nnc(Cl)cc12]

To a stirred solution of intermediate 2 (1.2 g, 5.374 mmol, 1.00 equiv) and 4-bromo-6-chloropyridazin-3-amine (1.68 g, 8.061 mmol, 1.50 equiv) in DMF (30 mL) were added Pd(OAc)₂ (241.28 mg, 1.075 mmol, 0.20 equiv), LiCl (227.81 mg, 5.374 mmol, 1.00 equiv) and Na₂CO₃ (2.85 g, 26.870 mmol, 5.00 equiv) at room temperature under an atmosphere of dry nitrogen. The reaction was stirred overnight at 120° C. The crude product was purified by reverse phase flash chromatography to afford intermediate 3B (140 mg, 7.42%) and intermediate 3A (340 mg, 18.03%) as yellow solids. LCMS (ESI) m/z: [M+H]⁺=351.

### Step 3: Preparation of tert-butyl 4-[3-(2-hydroxyphenyl)-5-methyl-7H-pyrrolo[2,3-c]pyridazin-6-yl]piperidine-1-carboxylate (Intermediate 4)

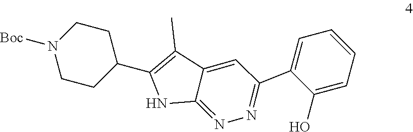 [SMILES: Cc1c(C2CCN(C(=O)OC(C)(C)C)CC2)[nH]c2nnc(-c3ccccc3O)cc12]

To a stirred mixture of intermediate 3A (320.00 mg, 0.912 mmol, 1.00 equiv) and 2-hydroxyphenylboronic acid (251.60 mg, 1.824 mmol, 2.00 equiv) in dioxane (6.0 mL) and H₂O (1.5 mL) were added Cs₂CO₃ (891.51 mg, 2.736 mmol, 3.00 equiv) and XPhos Pd G3 (154.40 mg, 0.182 mmol, 0.20 equiv) in portions at room temperature under an atmosphere of dry nitrogen. The resulting mixture was stirred for 2 h at 90° C. The residue was purified by reverse phase flash chromatography to afford intermediate 4 (240 mg, 64.42%) as a yellow solid. LCMS (ESI) m/z: [M+H]⁺=409.

### Step 4: Preparation of 2-[5-methyl-6-(piperidin-4-yl)-7H-pyrrolo[2,3-c]pyridazin-3-yl]phenol (Intermediate 5)

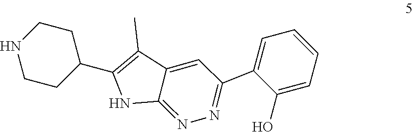 [SMILES: Cc1c(C2CCNCC2)[nH]c2nnc(-c3ccccc3O)cc12]

To a stirred solution of intermediate 4 (100 mg, 0.245 mmol, 1 equiv) in DCM (2.0 mL) was added TFA (0.5 mL) dropwise at room temperature. The resulting mixture was stirred for 1 h at room temperature. The resulting mixture was concentrated under reduced pressure. The crude product 5 was used in the next step directly without further purification. LCMS (ESI) m/z: [M+H]⁺=309.

### Step 5: Preparation of (2S,4R)-4-hydroxy-1-[(2R)-2-[3-(2-{4-[3-(2-hydroxyphenyl)-5-methyl-7H-pyrrolo[2,3-c]pyridazin-6-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 270)

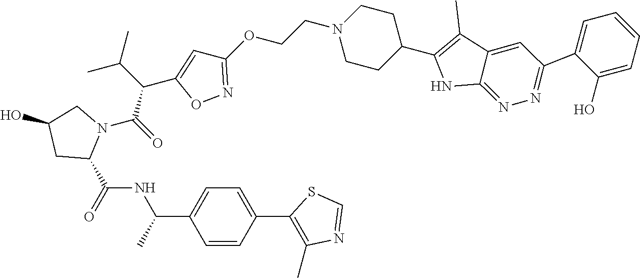 [SMILES: Cc1ncsc1-c1ccc([C@H](C)NC(=O)[C@@H]2C[C@@H](O)CN2C(=O)[C@@H](c2cc(OCCN3CCC(c4[nH]c5nnc(-c6ccccc6O)cc5c4C)CC3)no2)C(C)C)cc1]

To a stirred solution of intermediate 5 (11.41 mg, 0.036 mmol, 2 equiv) and (2S,4R)-4-hydroxy-N-[(1S)-1-[4-(4-methyl-1,3-thiazol-5-yl)phenyl]ethyl]-1-[(2R)-3-methyl-2-[3-(2-oxoethoxy)-1,2-oxazol-5-yl]butanoyl]pyrrolidine-2-carboxamide (10 mg, 0.018 mmol, 1.00 equiv) in MeOH (1.0 mL) and DCM (1.0 mL) were added AcOH (cat.) and NaBH₃CN (5.81 mg, 0.090 mmol, 5 equiv) in portions at room temperature. The resulting mixture was stirred overnight at room temperature. The resulting mixture was concentrated under reduced pressure. The crude product was purified by Prep-HPLC to afford compound 270 (3.0 mg, 19.47%) as a yellow solid. ¹H NMR (400 MHz, Methanol-d₄) δ 8.87 (s, 1H), 8.37 (s, 1H), 8.02-7.91 (m, 1H), 7.51-7.38 (m, 3H), 7.35 (s, 1H), 7.27 (ddd, J=8.6, 7.3, 1.6 Hz, 1H), 6.96 (dtt, J=7.1, 4.7, 2.3 Hz, 2H), 6.04 (s, 1H), 5.03 (q, J=7.0 Hz, 1H), 4.52 (t, J=8.2 Hz, 1H), 4.48-4.36 (m, 3H), 3.85 (dd, J=10.9, 4.2 Hz, 1H), 3.79-3.65 (m, 1H), 3.64-3.60 (m, 1H), 3.59-3.45 (m, 1H), 3.20 (d, J=11.2 Hz, 2H), 3.06 (s, 1H), 2.90 (t, J=5.3 Hz, 2H), 2.47 (s, 2H), 2.41 (s, 1H), 2.34 (d, J=9.5 Hz, 6H), 2.24-2.13 (m, 1H), 2.09-1.91 (m, 3H), 1.88 (d, J=12.6 Hz, 2H), 1.60-1.51 (m, 3H), 1.06 (dd, J=6.6, 1.8 Hz, 3H), 0.91 (dd, J=8.8, 6.7 Hz, 3H). LCMS (ESI) m/z: [M+H]⁺=834.2.

### Preparation of (2S,4R)-4-hydroxy-1-[(2R)-2-[3-(2-{4-[3-(2-hydroxyphenyl)-6-methyl-7H-pyrrolo[2,3-c]pyridazin-5-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 265)

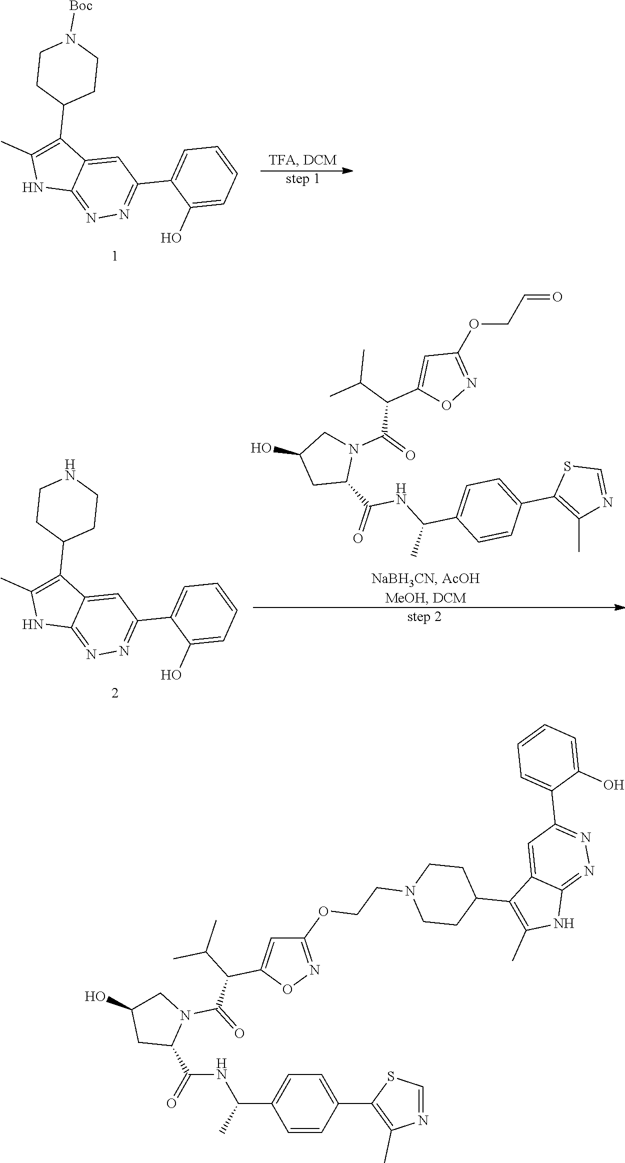 [SMILES: Cc1[nH]c2nnc(-c3ccccc3O)cc2c1C1CCN(C(=O)OC(C)(C)C)CC1 ; Cc1[nH]c2nnc(-c3ccccc3O)cc2c1C1CCNCC1 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCC(c4c(C)[nH]c5nnc(-c6ccccc6O)cc45)CC3)no2)C(C)C)cc1]

### Step 1: Preparation of 2-[6-methyl-5-(piperidin-4-yl)-7H-pyrrolo[2,3-c]pyridazin-3-yl]phenol (Intermediate 2)

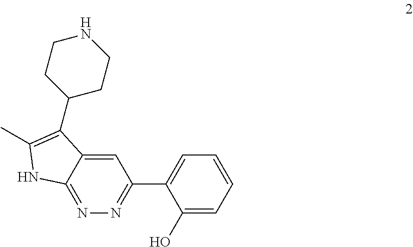 [SMILES: Cc1[nH]c2nnc(-c3ccccc3O)cc2c1C1CCNCC1]

To a stirred solution of tert-butyl 4-(3-(2-hydroxyphenyl)-6-methyl-7H-pyrrolo[2,3-c]pyridazin-5-yl)piperidine-1-carboxylate (20 mg, 0.049 mmol, 1 equiv) in DCM (1.0 mL) was added TEA (0.25 mL) dropwise at room temperature. The resulting mixture was stirred for 1 h at room temperature. The resulting mixture was concentrated under reduced pressure. The crude product 2 was used in the next step directly without further purification. LCMS (ESI) m/z: [M+H]⁺=309.

### Step 2: Preparation of (2S,4R)-4-hydroxy-1-[(2R)-2-[3-(2-{4-[3-(2-hydroxyphenyl)-6-methyl-7H-pyrrolo[2,3-c]pyridazin-5-yl]piperidin-1-yl}ethoxy)-1,2-oxazol-5-yl]-3-methylbutanoyl]-N-[(1S)-1-[4-(4-methyl-1,3-thiazol-5-yl)phenyl]ethyl]pyrrolidine-2-carboxamide (Compound 265)

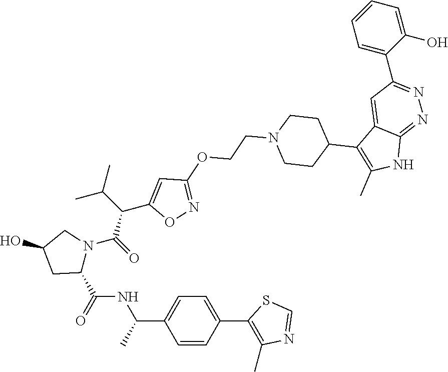 [SMILES: Cc1ncsc1-c1ccc([C@H](C)NC(=O)[C@@H]2C[C@@H](O)CN2C(=O)[C@@H](c2cc(OCCN3CCC(c4c(C)[nH]c5nnc(-c6ccccc6O)cc45)CC3)no2)C(C)C)cc1]

To a stirred solution of intermediate 2 (14.03 mg, 0.046 mmol, 2 equiv) and (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (12.3 mg, 0.023 mmol, 1 equiv) in MeOH (1.0 mL) and DCM (1.0 mL) were added AcOH (cat.) and NaBH₃CN (7.1 mg, 0.113 mmol, 5 equiv) in portions at room temperature. The resulting mixture was stirred overnight at room temperature. The resulting mixture was concentrated under reduced pressure. The crude product was purified by Prep-HPLC to afford compound 265 (2.3 mg, 4.26%) as a yellow solid. ¹H NMR (300 MHz, Methanol-d₄) δ 8.88 (d, J=4.0 Hz, 1H), 8.75 (d, J=4.0 Hz, 1H), 8.08 (d, J=8.4 Hz, 1H), 7.51-7.35 (m, 4H), 7.35-7.24 (m, 1H), 6.99 (q, J=7.4, 6.7 Hz, 2H), 6.09 (s, 1H), 5.05 (d, J=7.1 Hz, 1H), 4.59-4.48 (m, 1H), 4.47-4.39 (m, 3H), 3.93-3.81 (m, 1H), 3.71 (d, J=9.9 Hz, 1H), 3.65 (d, J=1.9 Hz, 3H), 3.64-3.47 (m, 1H), 3.24 (d, J=7.8 Hz, 2H), 2.94 (t, J=5.2 Hz, 2H), 2.56-2.51 (m, 3H), 2.50-2.48 (m, 2H), 2.47-2.28 (m, 6H), 2.19 (dd, J=14.4, 7.2 Hz, 1H), 2.10-1.75 (m, 4H), 1.62-1.58 (m, 1H), 1.56-1.50 (m, 3H), 1.19-1.04 (m, 3H), 0.93 (t, J=6.9 Hz, 3H). LCMS (ESI) m/z: [M+H]⁺=834.40.

The compounds in Table 18 were prepared using procedures similar to those used above for the preparation of compound 265 using the appropriate amine and aldehyde.

TABLE 18

| | | LCMS | |
|---|---|---|---|
| | | (ESI) | |
| No. | Name | m/z | ¹H NMR |
| 108 | (2S,4R)-4-hydroxy-1-((R)-2-(3-(2- | 859.30 | 1H NMR (400 MHz, Methanol-d4) δ 8.85 (d, J = |
| | ((3aR,5R,6aS)-5-(3-(2- | | 15.3 Hz, 1H), 8.35 (d, J = 10.9 Hz, 1H), 7.99 |
| | hydroxyphenyl)-5-methyl-7H- | | (t, J = 7.9 Hz, 1H), 7.44-7.39 (m, 4H), 7.27 (t, |
| | pyrrolo[2,3-c]pyridazin-6- | | J = 7.7 Hz, 1H), 7.01-6.92 (m, 2H), 6.05 (s, |
| | yl)hexahydrocyclopenta[c]pyrrol- | | 1H), 5.03-5.02 (m, 1H), 4.51 (t, J = 8.2 Hz, |
| | 2(1H)-yl)ethoxy)isoxazol-5-yl)-3- | | 1H), 4.46-4.34 (m, 3H), 3.83-3.70 (m, 1H), |
| | methylbutanoyl)-N-((S)-1-(4-(4- | | 3.70-3.60 (m, 3H), 3.62 (d, J = 10.8 Hz, 1H), |
| | methylthiazol-5- | | 3.02-2.97 (m, 4H), 2.47 (s, 3H), 2.44-2.35 |
| | yl)phenyl)ethyl)pyrrolidine-2- | | (m, 6H), 2.20 (t, J = 12.2 Hz, 1H), 2.10-2.01 |
| | carboxamide | | (m, 2H), 1.98-1.91 (m, 3H), 1.52 (d, J = 7.0 |
| | | | Hz, 3H), 1.06 (d, J = 6.5 Hz, 3H), 0.91 (d, J = |
| | | | 9.4, 6.6 Hz, 4H). |
| 109 | (2S,4R)-4-hydroxy-1-((R)-2-(3-(2- | 859.20 | 1H NMR (300 MHz, Methanol-d4) δ 8.88 (d, J = |
| | ((3aR,5R,6aS)-5-(3-(2- | | 5.5 Hz, 1H), 8.57 (d, J = 6.2 Hz, 1H), 8.05- |
| | hydroxyphenyl)-6-methyl-7H- | | 7.94 (m, 1H), 7.44 (d, J = 3.4 Hz, 3H), 7.38- |
| | pyrrolo [2,3-c]pyridazin-5- | | 7.23 (m, 2H), 7.03-6.94 (m, 2H), 6.01-5.88 |
| | yl)hexahydrocyclopenta[c]pyrrol- | | (m, 1H), 5.10-4.99 (m, 1H), 4.54-4.41 (m, |
| | 2(1H)-yl)ethoxy)isoxazol-5-yl)-3- | | 4H), 3.79-3.72 (m, 1H), 3.71-3.50 (m, 2H), |
| | methylbutanoyl)-N-((S)-1-(4-(4- | | 3.31-3.19 (m, 1H), 3.06-2.91 (m, 4H), 2.89- |
| | methylthiazol-5- | | 2.72 (m, 2H), 2.50-2.59 (m, 7H), 2.42 (s, |
| | yl)phenyl)ethyl)pyrrolidine-2- | | 1H), 2.40-2.15 (m, 4H), 2.08-1.91 (m, 3H), |
| | carboxamide | | 1.53 (d, J = 7.1 Hz, 3H), 1.06 (d, J = 6.5 Hz, |
| | | | 3H), 0.88 (dd, J = 12.2, 6.6 Hz, 3H). |
| 112 | (2S,4R)-4-hydroxy-1-((R)-2-(3-(2- | 859.30 | 1H NMR (300 MHz, Methanol-d4) δ 8.96 (s, |
| | ((3aR,5S,6aS)-5-(3-(2- | | 1H), 8.42 (d, J = 16.6 Hz, 1H), 8.08 (d, J = 7.4 |
| | hydroxyphenyl)-5-methyl-7H- | | Hz, 1H), 7.51-7.50 (m, 3H), 7.43-7.30 (m, |
| | pyrrolo[2,3-c]pyridazin-6- | | 2H), 7.11-7.00 (m, 2H), 6.15 (s, 1H), 5.11 (d, |
| | yl)hexahydrocyclopenta[c]pyrrol- | | J = 7.0 Hz, 1H), 4.66-4.49 (m, 4H), 3.98- |
| | 2(1H)-yl)ethoxy)isoxazol-5-yl)-3- | | 3.68 (m, 3H), 3.67-3.59 (m, 2H), 3.14-3.03 |
| | methylbutanoyl)-N-((S)-1-(4-(4- | | (m, 4H), 2.96 (s, 2H), 2.63 (s, 2H), 2.56 (s, |
| | methylthiazol-5- | | 3H), 2.52-2.39 (m, 7H), 2.11-2.01(m, 1H), |
| | yl)phenyl)ethyl)pyrrolidine-2- | | 1.93 (s, 3H), 1.64 (dd, J = 20.0, 7.0 Hz, 3H), |
| | carboxamide | | 1.14 (d, J = 6.6 Hz, 3H), 0.98 (t, J = 7.4 Hz, |
| | | | 3H). |
| 113 | (2S,4R)-4-hydroxy-1-((R)-2-(3-(2- | 859.30 | 1H NMR (300 MHz, DMSO-d6) δ 14.35 (s, |
| | ((3aR,5S,6aS)-5-(3-(2- | | 1H), 12.35 (s, 1H), 9.06 (s, 1H), 8.61-8.53 |
| | hydroxyphenyl)-6-methyl-7H- | | (m, 1H), 8.52-8.43 (m, 1H), 8.24 (d, J = 7.8 |
| | pyrrolo[2,3-c]pyridazin-5- | | Hz, 1H), 7.61-7.49 (m, 2H), 7.47-7.41 (m, |
| | yl)hexahydrocyclopenta[c]pyrrol- | | 2H), 7.40-7.28 (m, 1H), 7.03 (t, J = 7.1 Hz, |
| | 2(1H)-yl)ethoxy)isoxazol-5-yl)-3- | | 2H), 6.19-6.00 (m, 1H), 5.24-5.08 (m, 1H), |
| | methylbutanoyl)-N-((S)-1-(4-(4- | | 4.99 (t, J = 7.2 Hz, 1H), 4.45 (t, J = 7.9 Hz, |
| | methylthiazol-5- | | 1H), 4.39-4.24 (m, 3H), 3.88-3.63 (m, 2H), |
| | yl)phenyl)ethyl)pyrrolidine-2- | | 3.62-3.49 (m, 2H), 3.13-2.98 (m, 2H), 2.97- |
| | carboxamide | | 2.75 (m, 4H), 2.57-2.52 (m, 5H), 2.40- |
| | | | 2.20 (m, 5H), 2.18-2.03 (m, 1H), 2.02-1.66 |
| | | | (m, 3H), 1.62-1.37 (m, 3H), 1.04 (d, J = 6.4 |
| | | | Hz, 3H), 0.89 (t, J = 8.6 Hz, 3H). |
| 125 | (2S,4R)-1-((R)-2-(3-(2-((1R,5S,6R)- | 867.20 | 1H NMR (300 MHz, DMSO-d6) δ 15.11 (s, |
| | 6-(3-(3,5-difluoro-2-hydroxyphenyl)- | | 1H), 12.07 (s, 1H), 8.98 (d, J = 5.0 Hz, 1H), |
| | 5-methyl-7H-pyrrolo[2,3-c]pyridazin- | | 8.62 (s, 1H), 8.43 (d, J = 7.7 Hz, 1H), 7.93 (d, |
| | 6-yl)-3-azabicyclo[3.1.0]hexan-3- | | J = 10.2 Hz, 1H), 7.44 (d, J = 8.1 Hz, 2H), |
| | yl)ethoxy)isoxazol-5-yl)-3- | | 7.37 (d, J = 8.2 Hz, 2H), 6.13 (s, 1H), 5.12 (d, |
| | methylbutanoyl)-4-hydroxy-N-((S)- | | J = 3.6 Hz, 1H), 4.92 (t, J = 7.1 Hz, 1H), 4.38 |
| | 1-(4-(4-methylthiazol-5- | | (t, J = 8.0 Hz, 1H), 4.28 (d, J = 5.6 Hz, 3H), |
| | yl)phenyl)ethyl)pyrrolidine-2- | | 3.69-3.65 (m, 2H), 3.59-3.38 (m, 2H), 3.24 |
| | carboxamide | | (d, J = 9.1 Hz, 2H), 2.88 (s, 2H), 2.58 (s, 2H), |
| | | | 2.45 (d, J = 4.6 Hz, 4H), 2.32 (d, J = 3.4 Hz, |
| | | | 3H), 2.28 (s, 1H), 2.20 (s, 2H), 2.04 (s, 1H), |
| | | | 1.84-1.73 (m, 1H), 1.47-1.37 (m, 3H), 0.97 |
| | | | (d, J = 6.5 Hz, 3H), 0.81 (d, J = 6.8 Hz, 3H). |
| 126 | (2S,4R)-1-((R)-2-(3-(2-((1R,5S,6R)- | 849.30 | 1H NMR (300 MHz, Methanol-d4) δ 8.89 (s, |
| | 6-(3-(3-fluoro-2-hydroxyphenyl)-5- | | 1H), 8.33 (d, J = 13.9 Hz, 1H), 7.82 (d, J = 8.3 |
| | methyl-7H-pyrrolo[2,3-c]pyridazin-6- | | Hz, 1H), 7.45 (d, J = 2.8 Hz, 3H), 7.39 (d, J = |
| | yl)-3-azabicyclo[3.1.0]hexan-3- | | 15.3 Hz, 1H), 7.20-7.08 (m, 1H), 6.93-6.92 |
| | yl)ethoxy)isoxazol-5-yl)-3- | | (m, 1H), 6.06 (s, 1H), 5.10-4.96 (m, 1H), 4.54 |
| | methylbutanoyl)-4-hydroxy-N-((S)- | | (t, J = 8.3 Hz, 1H), 4.46 (s, 1H), 4.36 (t, J = 5.5 |
| | 1-(4-(4-methylthiazol-5- | | Hz, 2H), 3.91-3.81 (m, 1H), 3.69-3.67 (m, |
| | yl)phenyl)ethyl)pyrrolidine-2- | | 2H), 3.38 (s, 1H), 2.98 (t, J = 5.4 Hz, 2H), |
| | carboxamide | | 2.70 (d, J = 9.0 Hz, 2H), 2.65 (s, 1H), 2.47 (d, |
| | | | J = 15.1 Hz, 3H), 2.38 (d, J = 9.5 Hz, 4H), 2.17 |
| | | | (s, 3H), 2.10-1.88 (m, 1H), 1.62-1.53 (m, |
| | | | 3H), 1.08 (d, J = 6.5 Hz, 3H), 0.93 (d, J = 7.0 |
| | | | Hz, 3H). |
| 127 | (2S,4R)-1-((R)-2-(3-(2-(3-(3-(3- | 823.40 | 1H NMR (400 MHz, DMSO-d6) δ 15.10 (s, |
| | fluoro-2-hydroxyphenyl)-5-methyl- | | 1H), 12.54 (s, 1H), 9.01-8.96 (m, 1H), 8.66- |
| | 7H-pyrrolo[2,3-c]pyridazin-6- | | 8.60 (m, 1H), 8.41 (d, J = 7.7 Hz, 1H), 7.97 (d, |
| | yl)azetidin-1-yl)ethoxy)isoxazol-5- | | J = 8.2 Hz, 1H), 7.48-7.40 (m, 2H), 7.40- |
| | yl)-3-methylbutanoyl)-4-hydroxy-N- | | 7.33 (m, 2H), 7.29-7.20 (m, 1H), 6.97-6.87 |
| | ((S)-1-(4-(4-methylthiazol-5- | | (m, 1H), 6.09 (s, 1H), 5.11 (d, J = 3.6 Hz, 1H), |
| | yl)phenyl)ethyl)pyrrolidine-2- | | 4.92 (q, J = 7.0 Hz, 1H), 4.37 (t, J = 7.9 Hz, |
| | carboxamide | | 1H), 4.29 (s, 1H), 4.18 (t, J = 5.3 Hz, 2H), 4.05 |
| | | | (q, J = 8.1 Hz, 1H), 3.75 (t, J = 7.2 Hz, 2H), |
| | | | 3.73-3.68 (m, 1H), 3.66 (d, J = 9.8 Hz, 1H), |
| | | | 3.49-3.41 (m, 3H), 2.90 (t, J = 5.3 Hz, 2H), |
| | | | 2.48-2.42 (m, 3H), 2.30-2.25 (m, 3H), 2.29- |
| | | | 2.20 (m, 1H), 2.08-1.98 (m, 1H), 1.84- |
| | | | 1.73 (m, 1H), 1.41 (d, J = 7.0 Hz, 3H), 1.00- |
| | | | 0.93 (m, 3H), 0.82 (d, J = 6.7 Hz, 3H). |
| 128 | (2S,4R)-4-hydroxy-1-((R)-2-(3-(3- | 845.20 | 1H NMR (400 MHz, DMSO-d6) δ 14.28 (s, |
| | ((1R,5S,6S)-6-(3-(2- | | 1H), 12.28 (brs, 1H), 8.98 (s, 1H), 8.41 (d, J = |
| | hydroxyphenyl)-6-methyl-7H- | | 7.8 Hz, 1H), 8.36 (s, 1H), 8.17-8.11 (m, 1H), |
| | pyrrolo[2,3-c]pyridazin-5-yl)-3- | | 7.46-7.25 (m, 5H), 6.96 (t, J = 7.2 Hz, 2H), |
| | azabicyclo[3.1.0]hexan-3- | | 6.09 (s, 1H), 5.10 (d, J = 3.6 Hz, 1H), 4.90 (t, J = |
| | yl)propoxy)isoxazol-5-yl)-3- | | 7.1 Hz, 1H), 4.36 (t, J = 7.9 Hz, 1H), 4.32- |
| | methylbutanoyl)-N-((S)-1-(4-(4- | | 4.25 (m, 1H), 4.24-4.20(m, 1H), 3.71-3.61 |
| | methylthiazol-5- | | (m, 1H), 3.49-3.34 (m, 3H), 3.29-3.22(m, |
| | yl)phenyl)ethyl)pyrrolidine-2- | | 2H), 2.61-2.53(m, 2H), 2.46 (s, 3H), 2.44- |
| | carboxamide | | 2.37 (m, 2H), 2.31-2.17 (m, 2H), 2.10 (s, |
| | | | 1H), 2.05-1.95(m, 1H), 1.94-1.83 (m, 4H), |
| | | | 1.82-1.75 (m, 1H), 1.44 (d, J = 6.8 Hz, 1H), |
| | | | 1.37 (d, J = 7.0 Hz, 3H), 1.24 (s, 1H), 0.95 (d, |
| | | | J = 6.4 Hz, 3H), 0.75 (d, J = 6.4 Hz, 3H). |
| 129 | (2S,4R)-1-((R)-2-(3-(2-(3-(3-(3- | 857.40 | 1H NMR (400 MHz, DMSO-d6) δ 15.72 (s, |
| | chloro-5-fluoro-2-hydroxyphenyl)-5- | | 1H), 12.58 (s, 1H), 9.01-8.95 (m, 1H), 8.77- |
| | methyl-7H-pyrrolo[2,3-c]pyridazin-6- | | 8.72 (m, 1H), 8.41 (d, J = 7.6 Hz, 1H), 8.15- |
| | yl)azetidin-1-yl)ethoxy)isoxazol-5- | | 8.06 (m, 1H), 7.50-7.32 (m, 5H), 6.09 (s, |
| | yl)-3-methylbutanoyl)-4-hydroxy-N- | | 1H), 5.11 (d, J = 3.6 Hz, 1H), 4.95-4.87 (m, |
| | ((S)-1-(4-(4-methylthiazol-5- | | 1H), 4.37 (t, J = 7.9 Hz, 1H), 4.29 (s, 1H), 4.18 |
| | yl)phenyl)ethyl)pyrrolidine-2- | | (t, J = 5.3 Hz, 2H), 4.06 (p, J = 8.0 Hz, 1H), |
| | carboxamide | | 3.76 (t, J = 7.2 Hz, 2H), 3.73-3.68 (m, 1H), |
| | | | 3.66 (d, J = 9.8 Hz, 1H), 3.49-3.41 (m, 3H), |
| | | | 2.90 (t, J = 5.4 Hz, 2H), 2.48-2.42 (m, 3H), |
| | | | 2.31-2.20 (m, 3H), 2.23-2.18 (m, 1H), 2.07- |
| | | | 1.98 (m, 1H), 1.84-1.73 (m, 1H), 1.41 (dd, |
| | | | J = 29.6, 7.0 Hz, 3H), 1.00-0.93 (m, 3H), |
| | | | 0.82 (dd, J = 13.8, 6.7 Hz, 3H). |
| 130 | (2S,4R)-1-((R)-2-(3-(2-(3-(3-(5- | 823.40 | 1H NMR (400 MHz, DMSO-d6) δ 14.18 (s, |
| | fluoro-2-hydroxyphenyl)-5-methyl- | | 1H), 12.49 (s, 1H), 9.01-8.96 (m, 1H), 8.66- |
| | 7H-pyrrolo[2,3-c]pyridazin-6- | | 8.61 (m, 1H), 8.41 (d, J = 7.7 Hz, 1H), 8.05- |
| | yl)azetidin-1-yl)ethoxy)isoxazol-5- | | 7.97 (m, 1H), 7.48-7.40 (m, 2H), 7.40-7.33 |
| | yl)-3-methylbutanoyl)-4-hydroxy-N- | | (m, 2H), 7.18-7.08 (m, 1H), 7.00-6.92 (m, |
| | ((S)-1-(4-(4-methylthiazol-5- | | 1H), 6.09 (s, 1H), 5.11 (d, J = 3.6 Hz, 1H), |
| | yl)phenyl)ethyl)pyrrolidine-2- | | 4.91 (p, J = 6.9 Hz, 1H), 4.37 (t, J = 7.9 Hz, |
| | carboxamide | | 1H), 4.29 (s, 1H), 4.18 (t, J = 5.3 Hz, 2H), 4.05 |
| | | | (p, J = 7.9 Hz, 1H), 3.75 (t, J = 7.2 Hz, 2H), |
| | | | 3.73-3.67 (m, 1H), 3.66 (d, J = 9.8 Hz, 1H), |
| | | | 3.49-3.41 (m, 3H), 2.90 (t, J = 5.3 Hz, 2H), |
| | | | 2.48-2.43 (m, 3H), 2.30-2.25 (m, 3H), 2.29- |
| | | | 2.20 (m, 1H), 2.07-1.98 (m, 1H), 1.84- |
| | | | 1.73 (m, 1H), 1.41 (dd, J = 30.2, 7.0 Hz, 3H), |
| | | | 1.00-0.93 (m, 3H), 0.82 (dd, J = 13.9, 6.7 Hz, |
| | | | 3H). |
| 220 | (2S,4R)-4-hydroxy-1-((R)-2-(3-(2- | 831.50 | 1H NMR (400 MHz, Methanol-d4) δ 8.87 (s, |
| | ((3-(3-(2-hydroxyphenyl)-5-methyl- | | 1H), 8.37 (s, 1H), 8.01-7.95 (m, 1H), 7.47- |
| | 7H-pyrrolo[2,3-c]pyridazin-6- | | 7.39 (m, 3H), 7.33 (s, 1H), 7.30-7.23 (m, |
| | yl)bicyclo[1.1.1]pentan-1- | | 1H), 7.01-6.93 (m, 2H), 6.05 (s, 1H), 5.07- |
| | yl)amino)ethoxy)isoxazol-5-yl)-3- | | 4.96 (m, 1H), 4.55-4.28 (m, 4H), 3.91-3.79 |
| | methylbutanoyl)-N-((S)-1-(4-(4- | | (m, 1H), 3.77-3.58 (m, 2H), 3.11-3.00 (m, |
| | methylthiazol-5- | | 2H), 2.48 (s, 3H), 2.43-2.33 (m, 10H), 2.24- |
| | yl)phenyl)ethyl)pyrrolidine-2- | | 2.13 (m, 1H), 2.01-1.90 (m, 1H), 1.62-1.47 |
| | carboxamide | | (m, 3H), 1.06 (d, J = 6.6 Hz, 3H), 0.95-0.83 |
| | | | (m, 3H). |
| 232 | (2S,4R)-4-hydroxy-1-((R)-2-(3-(2- | 819.50 | 1H NMR (400 MHz, DMSO-d6) δ 14.30- |
| | ((S)-3-(3-(2-hydroxyphenyl)-6- | | 14.24 (m, 1H), 12.24 (s, 1H), 8.98 (s, 1H), |
| | methyl-7H-pyrrolo[2,3-c]pyridazin-5- | | 8.69-8.64 (m, 1H), 8.40 (d, J = 7.6 Hz, 1H), |
| | yl)pyrrolidin-1-yl)ethoxy)isoxazol-5- | | 7.96 (dd, J = 8.0, 1.6 Hz, 1H), 7.47-7.40 (m, |
| | yl)-3-methylbutanoyl)-N-((S)-1-(4- | | 2H), 7.40-7.30 (m, 2H), 7.33-7.22 (m, 1H), |
| | (4-methylthiazol-5- | | 6.99-6.90 (m, 2H), 5.99 (s, 1H), 5.08 (d, J = |
| | yl)phenyl)ethyl)pyrrolidine-2- | | 3.6 Hz, 1H), 4.90 (q, J = 7.2 Hz, 1H), 4.40- |
| | carboxamide | | 4.32 (m, 3H), 4.27 (s, 1H), 3.73-3.49 (m, |
| | | | 3H), 3.46-3.39 (m, 1H), 3.16-3.01 (m, 1H), |
| | | | 3.02-2.91 (m, 1H), 2.98-2.84 (m, 2H), 2.84- |
| | | | 2.71 (m, 1H), 2.63 (d, J = 8.2 Hz, 1H), 2.48- |
| | | | 2.43 (m, 3H), 2.26-2.22 (m, 3H), 2.07-1.97 |
| | | | (m, 1H), 1.93-1.84 (m, 1H), 1.83-1.72 (m, |
| | | | 1H), 1.44-1.34 (m, 3H), 0.99-0.91 (m, 3H), |
| | | | 0.77 (dd, J = 14.4, 6.7 Hz, 3H). |
| 241 | (2S,4R)-4-hydroxy-1-((R)-2-(3-(2- | 819.50 | 1H NMR (400 MHz, DMSO-d6) δ 14.29- |
| | ((R)-3-(3-(2-hydroxyphenyl)-6- | | 14.22 (m, 1H), 12.11 (s, 1H), 8.98 (s, 1H), |
| | methyl-7H-pyrrolo[2,3-c]pyridazin-5- | | 8.83-8.34 (m, 2H), 7.99-7.92 (m, 1H), 7.47- |
| | yl)pyrrolidin-1-yl)ethoxy)isoxazol-5- | | 7.40 (m, 2H), 7.39-7.30 (m, 2H), 7.33- |
| | yl)-3-methylbutanoyl)-N-((S)-1-(4- | | 7.22 (m, 1H), 7.00-6.89 (m, 2H), 6.00 (s, |
| | (4-methylthiazol-5- | | 1H), 5.09 (d, J = 3.7 Hz, 1H), 4.90 (p, J = 7.2 |
| | yl)phenyl)ethyl)pyrrolidine-2- | | Hz, 1H), 4.40-4.32 (m, 3H), 4.28 (s, 1H), |
| | carboxamide | | 3.74-3.50 (m, 3H), 3.46-3.39 (m, 1H), 3.12- |
| | | | 3.05 (m, 1H), 2.95-2.84 (m, 3H), 2.83- |
| | | | 2.73 (m, 1H), 2.67-2.61 (m, 1H), 2.48-2.43 |
| | | | (m, 3H), 2.28-2.13 (m, 3H), 2.06-1.97 (m, |
| | | | 1H), 1.95-1.84 (m, 1H), 1.83-1.72 (m, 1H), |
| | | | 1.46-1.34 (m, 3H), 0.98-0.91 (m, 3H), 0.83- |
| | | | 0.73 (m, 3H). |
| 254 | (2S,4R)-4-hydroxy-1-((R)-2-(3-(2-(3- | 805.50 | 1H NMR (400 MHz, DMSO-d6) δ 14.26- |
| | (3-(2-hydroxyphenyl)-6-methyl-7H- | | 14.22 (m, 1H), 12.33 (s, 1H), 8.98 (s, 1H), |
| | pyrrolo[2,3-c]pyridazin-5-yl)azetidin- | | 8.90-8.83 (m, 1H), 8.40 (d, J = 7.7 Hz, 1H), |
| | 1-yl)ethoxy)isoxazol-5-yl)-3- | | 8.02-7.94 (m, 1H), 7.47-7.40 (m, 2H), 7.40- |
| | methylbutanoyl)-N-((S)-1-(4-(4- | | 7.23 (m, 3H), 7.00-6.90 (m, 2H), 6.08 (s, |
| | methylthiazol-5- | | 1H), 5.09 (d, J = 3.7 Hz, 1H), 4.97-4.85 (m, |
| | yl)phenyl)ethyl)pyrrolidine-2- | | 1H), 4.37 (t, J = 7.8 Hz, 1H), 4.32-4.19 (m, |
| | carboxamide | | 3H), 3.89-3.79 (m, 1H), 3.78-3.49 (m, 4H), |
| | | | 3.45 (t, J = 6.1 Hz, 3H), 2.96-2.89 (m, 2H), |
| | | | 2.48-2.43 (m, 6H), 2.27-2.15 (m, 1H), 2.06- |
| | | | 1.98 (m, 1H), 1.83-1.72 (m, 1H), 1.45- |
| | | | 1.34 (m, 3H), 0.99-0.91 (m, 3H), 0.83-0.73 |
| | | | (m, 3H). |
| 255 | (2S,4R)-4-hydroxy-1-((R)-2-(3-(2- | 831.20 | 1H NMR (300 MHz, Methanol-d4) δ 8.88 (s, |
| | ((1R,5S,6S)-6-(3-(2- | | 1H), 8.30 (s, 1H), 8.04-7.89 (m, 1H), 7.49- |
| | hydroxyphenyl)-6-methyl-7H- | | 7.35 (m, 4H), 7.34-7.24 (m, 1H), 7.03-6.95 |
| | pyrrolo[2,3-c]pyridazin-5-yl)-3- | | (m, 2H), 6.08 (s, 1H), 5.09-4.98 (m, 1H), |
| | azabicyclo[3.1.0]hexan-3- | | 4.53 (t, J = 8.2 Hz, 1H), 4.49-4.37 (m, 3H), |
| | yl)ethoxy)isoxazol-5-yl)-3- | | 3.91-3.81 (m, 1H), 3.76-3.67 (m, 1H), 3.68- |
| | methylbutanoyl)-N-((S)-1-(4-(4- | | 3.59 (m, 1H), 3.60-3.47 (m, 2H), 3.24- |
| | methylthiazol-5- | | 3.07 (m, 2H), 3.07-2.84 (m, 2H), 2.56 (s, |
| | yl)phenyl)ethyl)pyrrolidine-2- | | 3H), 2.49 (s, 3H), 2.43-2.36 (m, 1H), 2.27- |
| | carboxamide | | 2.13 (m, 2H), 2.07-1.98 (m, 3H), 1.57 (d, J = |
| | | | 7.0 Hz, 3H), 1.08 (d, J = 6.6 Hz, 3H), 0.93 (d, J = |
| | | | 6.7 Hz, 3H). |
| 258 | (2S,4R)-4-hydroxy-1-((R)-2-(3-(2- | 819.45 | 1H NMR (400 MHz, DMSO-d6) δ 14.42 (s, |
| | ((S)-3-(3-(2-hydroxyphenyl)-5- | | 1H), 12.15 (s, 1H), 8.98 (s, 1H), 8.84-8.38 |
| | methyl-7H-pyrrolo[2,3-c]pyridazin-6- | | (m, 2H), 8.16-8.08 (m, 1H), 7.48-7.40 (m, |
| | yl)pyrrolidin-1-yl)ethoxy)isoxazol-5- | | 2H), 7.40-7.33 (m, 2H), 7.32-7.24 (m, 1H), |
| | yl)-3-methylbutanoyl)-N-((S)-1-(4- | | 6.99-6.90 (m, 2H), 6.12 (s, 1H), 5.10 (d, J = |
| | (4-methylthiazol-5- | | 3.6 Hz, 1H), 4.97-4.85 (m, 1H), 4.37 (t, J = |
| | yl)phenyl)ethyl)pyrrolidine-2- | | 7.9 Hz, 1H), 4.33-4.27 (m, 3H), 3.80-3.62 |
| | carboxamide | | (m, 3H), 3.61-3.42 (m, 1H), 3.08 (t, J = 8.4 |
| | | | Hz, 1H), 2.99-2.76 (m, 4H), 2.76-2.67 (m, |
| | | | 1H), 2.48-2.43 (m, 3H), 2.34-2.16 (m, 5H), |
| | | | 2.07-1.96 (m, 2H), 1.84-1.73 (m, 1H), 1.41 |
| | | | (dd, J = 28.1, 7.0 Hz, 3H), 1.00-0.93 (m, 3H), |
| | | | 0.88-0.77 (m, 3H). |
| 259 | (2S,4R)-4-hydroxy-1-((R)-2-(3-(2- | 819.45 | 1H NMR (400 MHz, DMSO-d6) δ 14.45- |
| | ((R)-3-(3-(2-hydroxyphenyl)-5- | | 14.39 (m, 1H), 12.14 (s, 1H), 9.01-8.96 (m, |
| | methyl-7H-pyrrolo[2,3-c]pyridazin-6- | | 1H), 8.86-8.34 (m, 2H), 8.16-8.08 (m, 1H), |
| | yl)pyrrolidin-1-yl)ethoxy)isoxazol-5- | | 7.48-7.40 (m, 2H), 7.40-7.33 (m, 2H), 7.32- |
| | yl)-3-methylbutanoyl)-N-((S)-1-(4- | | 7.24 (m, 1H), 6.99-6.90 (m, 2H), 6.12 (s, |
| | (4-methylthiazol-5- | | 1H), 5.10 (d, J = 3.6 Hz, 1H), 4.97-4.85 (m, |
| | yl)phenyl)ethyl)pyrrolidine-2- | | 1H), 4.71-4.33 (m, 1H), 4.33-4.27 (m, 3H), |
| | carboxamide | | 3.80-3.62 (m, 3H), 3.49-3.41 (m, 1H), 3.08 |
| | | | (t, J = 8.3 Hz, 1H), 2.99-2.77 (m, 4H), 2.76- |
| | | | 2.67 (m, 1H), 2.48-2.43 (m, 3H), 2.34-2.15 |
| | | | (m, 5H), 2.07-1.96 (m, 2H), 1.83-1.73 (m, |
| | | | 1H), 1.41 (dd, 3H), 1.00-0.93 (m, 3H), 0.88- |
| | | | 0.76 (m, 3H). |
| 271 | (2S,4R)-4-hydroxy-1-((R)-2-(3-(2-(4- | 888.40 | 1H NMR (400 MHz, DMSO-d6) δ 14.39 (s, |
| | (3-(3-(2-hydroxyphenyl)-5-methyl- | | 1H), 12.34 (s, 1H), 8.99 (s, 1H), 8.57 (s, 1H), |
| | 7H-pyrrolo[2,3-c]pyridazin-6- | | 8.43 (d, J = 7.7 Hz, 1H), 8.12 (d, 1H), 7.50- |
| | yl)azetidin-1-yl)piperidin-1- | | 7.41 (m, 2H), 7.41-7.33 (m, 2H), 7.28 (td, |
| | yl)ethoxy)isoxazol-5-yl)-3- | | 1H), 6.99-6.91 (m, 2H), 6.11 (s, 1H), 5.12 (d, |
| | methylbutanoyl)-N-((S)-1-(4-(4- | | J = 3.5 Hz, 1H), 4.91 (t, J = 7.1 Hz, 1H), 4.37 |
| | methylthiazol-5- | | (t, J = 7.9 Hz, 1H), 4.31-4.20 (m, 3H), 3.98 |
| | yl)phenyl)ethyl)pyrrolidine-2- | | (p, J = 8.1 Hz, 1H), 3.74-3.62 (m, 4H), 3.53- |
| | carboxamide | | 3.41 (m, 1H), 3.30-3.27 (m, 2H), 2.85-2.78 |
| | | | (m, 2H), 2.68-2.65 (m, 2H), 2.46 (s, 3H), |
| | | | 2.28 (s, 3H), 2.26-2.21 (m, 2H), 2.14-1.99 |
| | | | (m, 3H), 1.83-1.73 (m, 1H), 1.69-1.61 (m, |
| | | | 2H), 1.48-1.35 (m, 3H), 1.25-1.15 (m, 2H), |
| | | | 1.01-0.93 (m, 3H), 0.88-0.77 (m, 3H). |
| 272 | (2S,4R)-4-hydroxy-1-((R)-2-(3-(2- | 831.00 | 1H NMR (300 MHz, DMSO-d6) δ 14.56 (br s, |
| | ((1R,5S,6R)-6-(3-(2- | | 1H), 11.89 (br s, 1H), 8.98 (d, J = 4.4 Hz, 1H), |
| | hydroxyphenyl)-5-methyl-7H- | | 8.48 (s, 1H), 8.42 (d, J = 7.7 Hz, 1H), 8.11 (dd, |
| | pyrrolo[2,3-c]pyridazin-6-yl)-3- | | J = 8.3, 1.6 Hz, 1H), 7.45 (d, J = 8.2 Hz, 2H), |
| | azabicyclo[3.1.0]hexan-3- | | 7.39 (d, J = 8.1 Hz, 2H), 7.32-7.21 (m, 1H), |
| | yl)ethoxy)isoxazol-5-yl)-3- | | 7.01-6.88 (m, 2H), 6.05 (d, J = 52.3 Hz, 1H), |
| | methylbutanoyl)-N-((S)-1-(4-(4- | | 5.19-5.00 (m, 1H), 4.99-4.86 (m, 1H), 4.38 |
| | methylthiazol-5- | | (t, J = 7.9 Hz, 1H), 4.33-4.21 (m, 3H), 3.79- |
| | yl)phenyl)ethyl)pyrrolidine-2- | | 3.66 (m, 1H), 3.65 (s, 1H), 3.52-3.41 (m, 1H), |
| | carboxamide | | 3.23 (d, J = 9.1 Hz, 2H), 2.88 (t, J = 5.5 Hz, |
| | | | 2H), 2.62-2.54 (m, 2H), 2.47-2.42 (m, 4H), |
| | | | 2.31 (d, J = 2.7 Hz, 3H), 2.29-2.25 (m, 1H), |
| | | | 2.20-2.18 (m, 2H), 2.11-1.97 (m, 1H), 1.87- |
| | | | 1.72 (m, 1H), 1.43 (dd, J = 23.5, 7.0 Hz, 3H), |
| | | | 1.05-0.91 (m, 3H), 0.88-0.75 (m, 3H). |
| 307 | (2S,4R)-4-hydroxy-1-((R)-2-(3-(2-(3- | 805.45 | 1H NMR (400 MHz, DMSO-d6) δ 14.32 (s, |
| | (3-(2-hydroxyphenyl)-5-methyl-7H- | | 1H), 12.48 (s, 1H), 8.98 (s, 1H), 8.59 (s, 1H), |
| | pyrrolo[2,3-c]pyridazin-6-yl)azetidin- | | 8.40 (d, J = 7.7 Hz, 1H), 8.13 (d, J = 1.9 Hz, |
| | 1-yl)ethoxy)isoxazol-5-yl)-3- | | 1H), 7.48-7.41 (m, 2H), 7.36 (d, J = 8.2 Hz, |
| | methylbutanoyl)-N-((S)-1-(4-(4- | | 2H), 7.28 (td, 1H), 6.95 (dd, J = 8.0, 6.7 Hz, |
| | methylthiazol-5- | | 2H), 6.11 (s, 1H), 5.11 (d, J = 3.6 Hz, 1H), |
| | yl)phenyl)ethyl)pyrrolidine-2- | | 4.92 (q, J = 7.0 Hz, 1H), 4.39-4.34 (m, 1H), |
| | carboxamide | | 4.31-4.20 (m, 3H), 4.14-4.07 (m, 1H), 3.89- |
| | | | 3.77 (m, 2H), 3.73-3.65 (m, 2H), 3.55- |
| | | | 3.42 (m, 3H), 3.09-2.89 (m, 2H), 2.45 (s, |
| | | | 3H), 2.28 (s, 3H), 2.24-2.17 (m, 1H), 2.07- |
| | | | 1.99 (m, 1H), 1.82-1.74 (m, 1H), 1.38 (d, J = |
| | | | 7.0 Hz, 3H), 0.96 (d, J = 6.4 Hz, 3H), 0.82 (dd, |
| | | | J = 14.0, 6.7 Hz, 3H). |

### Preparation of (2S,4R)-4-hydroxy-1-[(2R)-2-[3-(4-{3-[3-(2-hydroxyphenyl)-7H-pyrrolo[2,3-c]pyridazin-6-yl]azetidine-1-carbonyl}piperazin-1-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide (compound 124)

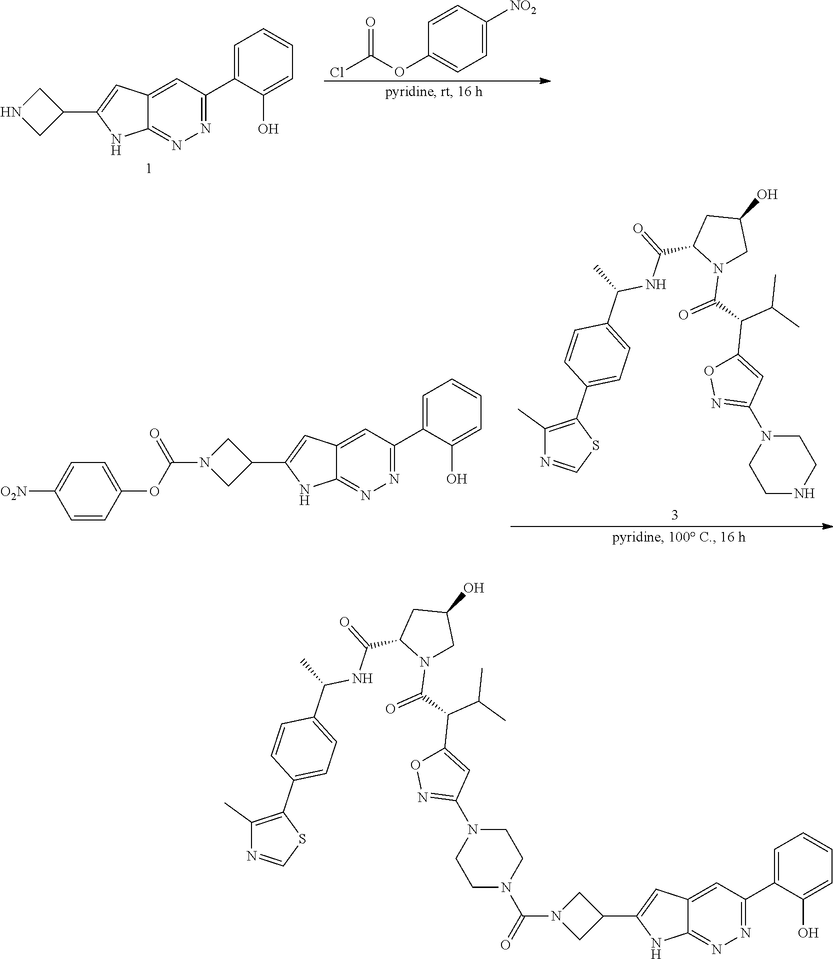

### Step 1: Preparation of 4-nitrophenyl 3-[3-(2-hydroxyphenyl)-7H-pyrrolo[2,3-c]pyridazin-6-yl]azetidine-1-carboxylate

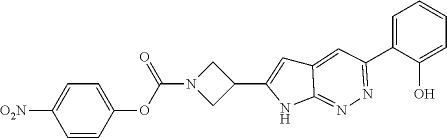 [SMILES: O=C(Oc1ccc([N+](=O)[O-])cc1)N1CC(c2cc3cc(-c4ccccc4O)nnc3[nH]2)C1]

To a stirred solution of 2-[6-(azetidin-3-yl)-7H-pyrrolo[2,3-c]pyridazin-3-yl]phenol (100.00 mg, 0.376 mmol, 1.00 equiv) in pyridine (5.00 mL) was added 4-nitrophenyl carbonochloridate (151.38 mg, 0.752 mmol, 2.00 equiv) at 0 degrees C. The resulting mixture was stirred for 16 h at room temperature. The resulting mixture was concentrated under vacuum. The residue was purified by reverse phase flash chromatography with the following conditions: column, C18 silica gel; mobile phase, MeCN in water (0.1% FA), 0% to 100% gradient in 30 min; detector, UV 254/220 nm. This resulted in intermediate 2 (59 mg, 36.42%) as a brown solid. LCMS (ESI) m/z: [M+H]⁺=432.

### Step 2: Preparation of (2S,4R)-4-hydroxy-1-[(2R)-2-[3-(4-{3-[3-(2-hydroxyphenyl)-7H-pyrrolo[2,3-c]pyridazin-6-yl]azetidine-1-carbonyl}piperazin-1-yl)-1,2-oxazol-5-yl]-3-methylbutanoyl]-N-[(1S)-1-[4-(4-methyl-1,3-thiazol-5-yl)phenyl]ethyl]pyrrolidine-2-carboxamide

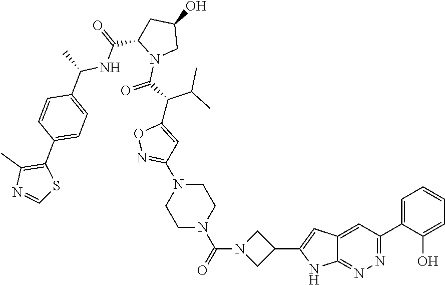 [SMILES: Cc1ncsc1-c1ccc([C@H](C)NC(=O)[C@@H]2C[C@@H](O)CN2C(=O)[C@@H](c2cc(N3CCN(C(=O)N4CC(c5cc6cc(-c7ccccc7O)nnc6[nH]5)C4)CC3)no2)C(C)C)cc1]

To a stirred solution of intermediate 2 (59.00 mg, 0.095 mmol, 1.00 equiv) in pyridine (5.00 mL) was added (2S,4R)-4-hydroxy-N-[(1S)-1-[4-(4-methyl-1,3-thiazol-5-yl)phenyl]ethyl]-1-[(2R)-3-methyl-2-[3-(piperazin-1-yl)-1,2-oxazol-5-yl]butanoyl]pyrrolidine-2-carboxamide (53.86 mg, 0.095 mmol, 1.00 equiv) at room temperature. The resulting mixture was stirred for 16 h at 100 degrees C. The mixture was allowed to cool down to room temperature. The resulting mixture was concentrated under reduced pressure. The crude product was purified by Prep-HPLC with the following conditions: Column, XBridge Prep Phenyl OBD Column, 19*150 mm, Sum; mobile phase, water (10 mmol/L NH₄HCO₃) and ACN (36% ACN up to 46% in 7 min); Detector, UV 254/220 nm. This resulted in compound 124 (1.00 mg, 1.13%) as a white solid. ¹H NMR (300 MHz, DMSO-d⁶) δ 14.12-14.04 (m, 1H), 12.78-12.60 (m, 1H), 8.99 (s, 1H), 8.59 (s, 1H), 8.43-8.31 (m, 1H), 8.08-8.01 (m, 1H), 7.49-7.41 (m, 2H), 7.41-7.34 (m, 2H), 7.34-7.26 (m, 1H), 7.01-6.92 (m, 2H), 6.66 (s, 1H), 6.19 (s, 1H), 5.13-5.07 (m, 1H), 4.98-4.86 (m, 1H), 4.44-4.24 (m, 4H), 4.24-4.07 (m, 3H), 3.77-3.67 (m, 1H), 3.65-3.53 (m, 1H), 3.53-3.35 (m, 5H), 3.23-3.12 (m, 4H), 2.46 (s, 3H), 2.26-2.11 (m, 1H), 2.10-1.96 (m, 1H), 1.86-1.73 (m, 1H), 1.39 (d, J=7.1 Hz, 3H), 0.96 (d, J=6.7 Hz, 3H), 0.80 (d, J=6.7 Hz, 3H). LCMS (ESI) m/z: [M+H]⁺=859.60.

### Example 5. Degradation of BRM and BRG1 by Compounds of the Invention

This example demonstrates the ability of the compounds of the disclosure to degrade a HiBit-BRM or HiBit-BRG1 fusion protein in a cell-based degradation assay.

Procedure: A stable HeLa cell line expressing HiBiT-BRM was generated. On day 0, 5000 cells were seeded in 40 μL of media into each well of 384-well cell culture plates. On day 1, cells were treated with 120 nL DMSO or 120 nL of 3-fold serially DMSO-diluted compounds (10 points in duplicate with 30 μM as final top dose). Subsequently plates were incubated for 24 h in a standard tissue culture incubator and equilibrated at room temperature for 15 minutes. Nano-Glo HiBiT Lytic Detection System (Promega N3050) reagent was freshly prepared and 20 ul was added to each well. Upon addition of this LgBit-containing reagent, the HiBiT and LgBiT proteins associate to form the luminescent NanoBiT luciferase. The plates were shaken for 10 minutes at room temperature and the bioluminescence read using an EnVision plate reader (PerkinElmer).

For measurement of BRG1 degradation, a stable HeLa cell line expressing HiBit-BRG1 and LgBit was generated. The same protocol as above was then followed.

The degradation % was calculated using the following formula: % degradation=100%−100%×(Lum_{Sample}−Lum_{LC})/(Lum_{HC}−Lum_{LC}). DMSO treated cells are employed as High Control (HC) and 2 μM of a known BRM/BRG1 degrader standard treated cells are employed as Low Control (LC). The data was fit to a four parameter, non-linear curve fit to calculate IC₅₀ (μM) values as shown in Table 19.

Results: As shown in Table 19 below, the compounds of the invention degraded BRM and/or BRG1.

TABLE 19

| | BRM HiBit | BRM HiBit | BRMG1 HiBit | BRG1 HiBit |
|---|---|---|---|---|
| Compound | Degradation | Degradation | Degradation | Degradation |
| No. | IC50 (nM) | Maximum (%) | IC50 (nM) | Maximum (%) |
| 1 | ++ | A | NC | C |
| 2 | ++ | A | NC | C |
| 3 | + | B | NC | C |
| 4 | ++ | A | NC | C |
| 5 | ++ | A | NC | C |
| 6 | + | B | + | A |
| 7 | + | B | + | B |
| 8 | + | A | + | A |
| 9 | + | B | NC | C |
| 10 | + | B | NC | C |
| 11 | ++ | B | NC | C |
| 12 | ++ | A | NC | C |
| 13 | ++ | A | NC | C |
| 14 | +++ | A | NC | C |
| 15 | ++ | A | NC | C |
| 16 | NC | C | NC | C |
| 17 | NC | C | NC | C |
| 18 | NC | C | NC | C |
| 19 | NC | C | NC | C |
| 20 | NC | C | NC | C |
| 21 | NC | C | NC | C |
| 22 | NC | C | NC | C |
| 23 | NC | C | NC | C |
| 24 | NC | C | NC | C |
| 25 | NC | C | NC | C |
| 26 | NC | C | NC | C |
| 27 | NC | C | NC | C |
| 28 | NC | C | NC | C |
| 29 | NC | C | NC | C |
| 30 | NC | C | NC | C |
| 31 | NC | C | NC | C |
| 32 | NC | C | NC | C |
| 33 | NC | C | NC | C |
| 34 | NC | C | NC | C |
| 35 | NC | C | NC | C |
| 36 | NC | C | NC | C |
| 37 | NC | C | NC | C |
| 38 | NC | C | NC | C |
| 39 | NC | C | NC | C |
| 40 | NC | C | NC | C |
| 41 | NC | C | NC | C |
| 42 | NC | C | NC | C |
| 43 | NC | C | NC | C |
| 44 | NC | C | NC | C |
| 45 | NC | C | NC | C |
| 46 | NC | C | NC | C |
| 47 | NC | C | NC | C |
| 48 | +++ | A | NC | C |
| 49 | +++ | A | ++ | B |
| 50 | NC | C | NC | C |
| 51 | +++ | A | ++ | B |
| 52 | +++ | A | NC | C |
| 53 | +++ | A | +++ | A |
| 54 | +++ | A | ++ | B |
| 55 | NC | C | NC | C |
| 56 | + | B | NC | C |
| 57 | NC | C | NC | C |
| 58 | ++ | A | ++ | A |
| 59 | ++ | B | NC | C |
| 60 | NC | C | NC | C |
| 61 | + | A | + | A |
| 62 | NC | C | NC | C |
| 63 | NC | C | NC | C |
| 64 | ++ | B | NC | C |
| 65 | + | B | NC | C |
| 66 | NC | C | NC | C |
| 67 | ++++ | A | +++ | A |
| 68 | +++ | A | NC | C |
| 69 | +++ | A | NC | C |
| 70 | ++ | B | NC | C |
| 71 | ++ | A | NC | C |
| 72 | + | B | NC | C |
| 73 | NC | C | NC | C |
| 74 | +++ | A | NC | C |
| 75 | +++ | A | ++ | B |
| 76 | ++ | A | NC | C |
| 77 | +++ | A | ++ | A |
| 78 | ++ | A | NC | C |
| 79 | NC | C | NC | C |
| 80 | +++ | A | NC | C |
| 81 | +++ | A | ++ | A |
| 82 | ++++ | A | ++ | A |
| 83 | +++ | A | +++ | A |
| 84 | NC | C | NC | C |
| 85 | +++ | A | ++ | A |
| 86 | ++ | A | NC | C |
| 87 | NC | C | NC | C |
| 88 | NC | C | NC | C |
| 89 | NC | C | NC | C |
| 90 | + | A | NC | C |
| 91 | NC | C | NC | C |
| 92 | +++ | A | +++ | A |
| 93 | +++ | A | ++ | A |
| 94 | +++ | A | NC | C |
| 95 | + | B | NC | C |
| 96 | +++ | A | ++ | B |
| 97 | ++ | A | NC | C |
| 98 | +++ | A | NC | C |
| 99 | NC | C | NC | C |
| 100 | NC | C | NC | C |
| 101 | ++++ | A | +++ | A |
| 102 | ++ | A | NC | C |
| 103 | ++ | B | NC | C |
| 104 | ++ | B | NC | C |
| 105 | +++ | A | NC | C |
| 106 | +++ | A | NC | C |
| 107 | +++ | A | +++ | A |
| 108 | ++++ | A | +++ | A |
| 109 | +++ | A | NC | C |
| 110 | +++ | A | NC | C |
| 111 | +++ | A | ++ | B |
| 112 | ++++ | A | +++ | A |
| 113 | ++++ | A | +++ | B |
| 114 | NC | C | NC | C |
| 115 | NC | C | NC | C |
| 116 | NC | C | NC | C |
| 117 | NC | C | NC | C |
| 118 | +++ | A | NC | C |
| 119 | +++ | A | + | B |
| 120 | +++ | A | NC | C |
| 121 | ++ | A | NC | C |
| 122 | NC | C | NC | B |
| 123 | ++ | A | NC | C |
| 124 | +++ | A | ++ | A |
| 125 | +++ | A | NC | C |
| 126 | +++ | A | ++ | B |
| 127 | +++ | A | NC | C |
| 128 | ++++ | A | +++ | A |
| 129 | +++ | A | ++ | B |
| 130 | ++++ | A | ++ | A |
| 131 | +++ | A | ++ | B |
| 132 | +++ | A | NC | C |
| 133 | +++ | A | ++ | B |
| 134 | NC | C | NC | C |
| 135 | NC | C | NC | C |
| 136 | NC | C | NC | C |
| 137 | +++ | A | NC | C |
| 138 | +++ | A | NC | C |
| 139 | ++ | B | NC | C |
| 140 | NC | C | NC | C |
| 141 | +++ | A | NC | C |
| 142 | +++ | A | NC | C |
| 143 | ++ | A | NC | C |
| 144 | +++ | A | ++ | B |
| 145 | ++ | A | NC | C |
| 146 | +++ | A | ++ | A |
| 147 | +++ | A | ++ | B |
| 148 | NC | C | NC | C |
| 149 | NC | C | NC | C |
| 150 | +++ | A | ++ | B |
| 151 | ++ | A | NC | C |
| 152 | ++ | B | NC | C |
| 153 | NC | C | NC | C |
| 154 | NC | C | NC | C |
| 155 | NC | C | NC | C |
| 156 | +++ | A | NC | C |
| 157 | ++ | A | ++ | A |
| 158 | ++ | A | NC | C |
| 159 | +++ | A | ++ | A |
| 160 | ++ | A | NC | C |
| 161 | +++ | A | ++ | A |
| 162 | NC | C | + | B |
| 163 | +++ | A | ++ | B |
| 164 | +++ | A | ++ | A |
| 165 | ++ | A | NC | C |
| 166 | +++ | A | + | C |
| 167 | ++ | A | ++ | B |
| 168 | +++ | A | NC | C |
| 169 | ++ | A | ++ | B |
| 170 | NC | C | NC | C |
| 171 | NC | C | NC | C |
| 172 | +++ | A | NC | C |
| 173 | ++ | A | + | B |
| 174 | + | B | NC | C |
| 175 | + | A | NC | C |
| 176 | NC | C | NC | C |
| 177 | + | B | NC | C |
| 178 | NC | C | NC | C |
| 179 | ++ | A | NC | C |
| 180 | NC | C | NC | C |
| 181 | ++ | A | NC | C |
| 182 | NC | C | NC | C |
| 183 | ++ | A | NC | C |
| 184 | + | B | NC | C |
| 185 | ++ | A | + | A |
| 186 | +++ | A | ++ | A |
| 187 | +++ | A | NC | C |
| 188 | ++ | A | ++ | A |
| 189 | NC | C | NC | C |
| 190 | NC | C | NC | C |
| 191 | NC | C | NC | C |
| 192 | +++ | A | NC | C |
| 193 | +++ | A | ++ | B |
| 194 | NC | C | NC | C |
| 195 | ++ | B | NC | C |
| 196 | ++ | B | NC | C |
| 197 | NC | C | NC | C |
| 198 | ++ | A | ++ | A |
| 199 | +++ | A | NC | C |
| 200 | +++ | A | NC | C |
| 201 | NC | C | NC | C |
| 202 | NC | C | NC | C |
| 203 | ++ | A | NC | C |
| 204 | ++ | A | NC | C |
| 205 | NC | C | NC | C |
| 206 | NC | C | NC | C |
| 207 | NC | C | NC | C |
| 208 | +++ | A | NC | C |
| 209 | +++ | A | NC | C |
| 210 | NC | C | NC | C |
| 211 | +++ | A | ++ | A |
| 212 | ++ | A | NC | C |
| 213 | +++ | A | ++ | A |
| 214 | ++ | A | ++ | A |
| 215 | NC | C | NC | C |
| 216 | ++ | A | NC | C |
| 217 | +++ | A | +++ | A |
| 218 | NC | C | NC | C |
| 219 | ++ | A | NC | C |
| 220 | ++++ | A | +++ | A |
| 221 | ++ | B | NC | C |
| 222 | +++ | B | NC | C |
| 223 | +++ | A | ++ | A |
| 224 | +++ | A | + | B |
| 225 | +++ | A | NC | C |
| 226 | ++ | A | ++ | A |
| 227 | NC | C | NC | C |
| 228 | NC | C | NC | C |
| 229 | + | A | NC | C |
| 230 | + | B | NC | C |
| 231 | ++ | A | NC | C |
| 232 | + | C | NC | C |
| 233 | +++ | A | +++ | A |
| 234 | ++ | A | NC | C |
| 235 | +++ | A | +++ | A |
| 236 | NC | C | NC | C |
| 237 | NC | C | NC | C |
| 238 | +++ | A | NC | C |
| 239 | ++ | A | NC | C |
| 240 | ++ | A | + | B |
| 241 | +++ | B | NC | C |
| 242 | ++ | B | NC | C |
| 243 | ++ | A | + | B |
| 244 | ++ | A | NC | B |
| 245 | +++ | A | ++ | A |
| 246 | +++ | A | ++ | A |
| 247 | +++ | A | + | B |
| 248 | +++ | A | ++ | B |
| 249 | ++ | A | NC | C |
| 250 | ++ | A | NC | C |
| 251 | ++ | A | ++ | A |
| 252 | NC | C | NC | C |
| 253 | ++ | A | ++ | A |
| 254 | +++ | A | NC | C |
| 255 | +++ | A | + | C |
| 256 | ++ | A | NC | C |
| 257 | NC | C | NC | C |
| 258 | ++++ | A | +++ | A |
| 259 | ++++ | A | +++ | A |
| 260 | +++ | B | NC | C |
| 261 | NC | C | NC | C |
| 262 | +++ | B | NC | C |
| 263 | ++ | A | + | A |
| 264 | NC | C | NC | C |
| 265 | ++ | A | NC | C |
| 266 | +++ | A | ++ | A |
| 267 | ++ | A | NC | C |
| 268 | ++ | A | + | A |
| 269 | +++ | A | ++ | A |
| 270 | ++++ | A | +++ | A |
| 271 | ++++ | A | +++ | A |
| 272 | ++++ | A | +++ | A |
| 273 | ++ | A | NC | C |
| 274 | +++ | A | ++ | A |
| 275 | +++ | A | NC | C |
| 276 | +++ | A | + | B |
| 277 | ++ | A | NC | C |
| 278 | +++ | A | +++ | B |
| 279 | +++ | A | NC | C |
| 280 | +++ | A | NC | C |
| 281 | +++ | A | NC | C |
| 282 | +++ | A | NC | C |
| 283 | +++ | B | NC | C |
| 284 | +++ | A | ++ | A |
| 285 | ++ | A | ++ | B |
| 286 | +++ | A | NC | C |
| 287 | ++ | B | NC | C |
| 288 | ++ | A | NC | C |
| 289 | +++ | A | NC | C |
| 290 | +++ | A | ++ | B |
| 291 | +++ | B | NC | C |
| 292 | ++ | B | NC | C |
| 293 | ++ | A | + | B |
| 294 | ++ | A | NC | C |
| 295 | +++ | A | ++ | A |
| 296 | + | A | + | B |
| 297 | NC | C | NC | C |
| 298 | NC | C | NC | C |
| 299 | + | B | NC | C |
| 300 | ++ | A | NC | B |
| 301 | + | B | NC | C |
| 302 | +++ | A | +++ | A |
| 303 | NC | C | NC | C |
| 304 | +++ | A | ++ | A |
| 305 | + | A | NC | C |
| 306 | NC | C | NC | C |
| 307 | ++++ | A | +++ | A |
| 308 | ++++ | A | +++ | A |
| 309 | ++ | B | NC | C |
| 310 | +++ | A | ++ | B |

“+” indicates inhibitory effect of ≥ 1000 nM; “++” indicates inhibitory effect of ≥ 100 nM; “+++” indicates inhibitory effect of ≥ 10 nM; ++++” indicates inhibitory effect of < 10 nM; “NT” indicates not tested; “NC” indicates not calculated; “A” indicates maximum degradation ≥ 75%; “B” indicates maximum degradation ≥ 50%; and “C” indicates maximum degradation < 50%.

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are in the claims.

1. A compound, or a pharmaceutically acceptable salt thereof, having the structure of Formula I: 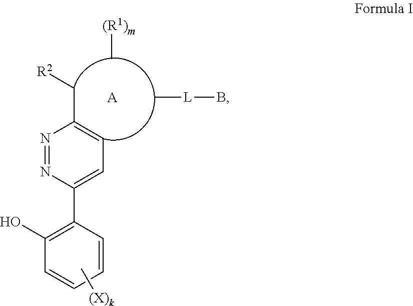
wherein
ring system A is a 5 to 9-membered heterocyclyl or heteroaryl;
m is 0, 1, 2, or 3;
k is 0, 1, or 2;
each R¹ is, independently, halo, optionally substituted C₁-C₆ alkyl, or optionally substituted C₃-C₈ cycloalkyl;
R² is H or optionally substituted C₁-C₆ alkyl;
each X is, independently, halo;
L is a linker; and
B is a degradation moiety.

2. The compound of claim 1, or a pharmaceutically acceptable salt thereof, wherein the compound has the structure of Formula I-A: 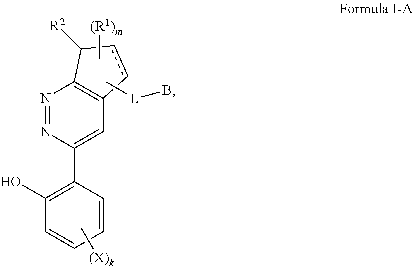
wherein the dashed bond represents a single or double bond.

3.-7. (canceled)

8. The compound of claim 1, or a pharmaceutically acceptable salt thereof, wherein R² is hydrogen and m is 0.

9. (canceled)

10. The compound of claim 1, or a pharmaceutically acceptable salt thereof, wherein the compound has the structure of Formula I-G or Formula I-H:

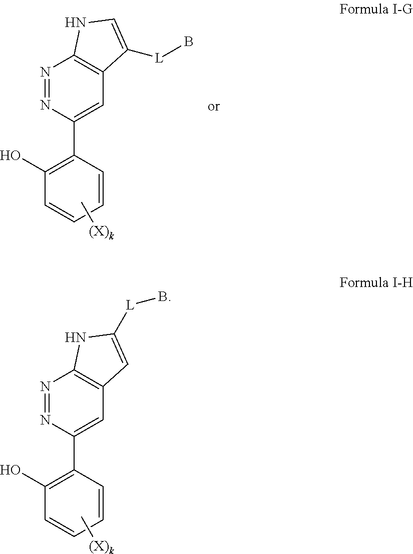

11. (canceled)

12. The compound of claim 1, or a pharmaceutically acceptable salt thereof, wherein the degradation moiety, B, has the structure of Formula A-1: 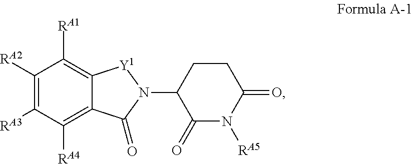
wherein
Y¹ is 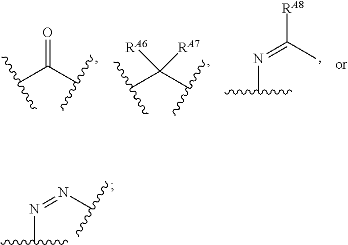
R^{A5} is H, optionally substituted C₁-C₆ alkyl, or optionally substituted C₁-C₆ heteroalkyl;
R^{A6} is H or optionally substituted C₁-C₆ alkyl; and R^{A7} is H or optionally substituted C₁-C₆ alkyl; or R^{A6} and R^{A7}, together with the carbon atom to which each is bound, combine to form optionally substituted C₃-C₆ carbocyclyl or optionally substituted C₂-C₅ heterocyclyl;
or R^{A6} and R^{A7}, together with the carbon atom to which each is bound, combine to form optionally substituted C₃-C₆ carbocyclyl or optionally substituted C₂-C₆ heterocyclyl;
R^{A8} is H, optionally substituted C₁-C₆ alkyl, or optionally substituted C₁-C₆ heteroalkyl;
each of R^{A1}, R^{A2}, R^{A3}, and R^{A4} is, independently, H, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, optionally substituted —O—C₃-C₆ carbocyclyl, hydroxyl, thiol, or optionally substituted amino; or R^{A1} and R^{A2}, R^{A2} and R^{A3}, and/or R^{A3} and R^{A4}, together with the carbon atoms to which each is attached, combine to form 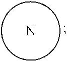
and 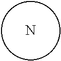
is optionally substituted C₆-C₁₀ aryl, optionally substituted C₃-C₁₀ carbocyclyl, optionally substituted C₂-C₉ heteroaryl, or C₂-C₉ heterocyclyl, any of which is optionally substituted with A²,
where one of R^{A1}, R^{A2}, R^{A3}, and R^{A4} is A², or 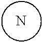
is substituted with A²; and
A² is a bond between the degradation moiety and the linker.

13. The compound of claim 12, or a pharmaceutically acceptable salt thereof, wherein R^{A5} is H or methyl, and each of R^{A1}, R^{A2}, R^{A3}, and R^{A4} is independently, H or A².

14.-19. (canceled)

20. The compound of claim 12, or a pharmaceutically acceptable salt thereof, wherein Y¹ is

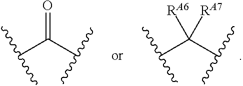

21. The compound of claim 20, or a pharmaceutically acceptable salt thereof, wherein R^{A6} is H or R^{A7} is H, or R^{A6} is H and R^{A7} is H.

22. (canceled)

23. The compound of claim 12, or a pharmaceutically acceptable salt thereof, wherein Y¹ is

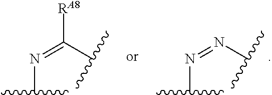

24. The compound of claim 23, or a pharmaceutically acceptable salt thereof, wherein R^{A8} is H or optionally substituted C₁-C₆ alkyl.

25.-27. (canceled)

28. The compound of claim 2, or a pharmaceutically acceptable salt thereof, wherein the degradation moiety is

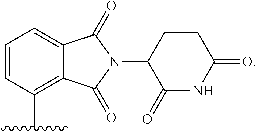 [SMILES: *c1cccc2c1C(=O)N(C1CCC(=O)NC1=O)C2=O]

29. (canceled)

30. The compound of claim 12, or a pharmaceutically acceptable salt thereof, wherein the degradation moiety is

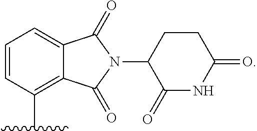 [SMILES: *c1cccc2c1C(=O)N(C1CCC(=O)NC1=O)C2=O]

31. The compound of to claim 12, or a pharmaceutically acceptable salt thereof, wherein the degradation moiety comprises the structure of Formula A5, Formula A6, Formula A8, or Formula A10:

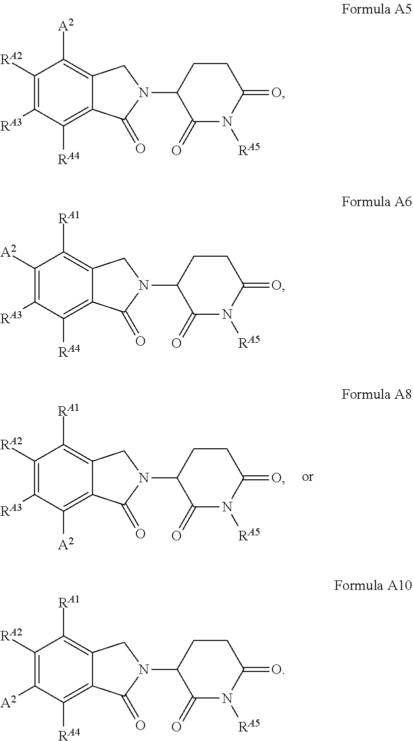

32.-34. (canceled)

35. The compound of claim 12, or a pharmaceutically acceptable salt thereof, wherein the degradation moiety comprises the structure of

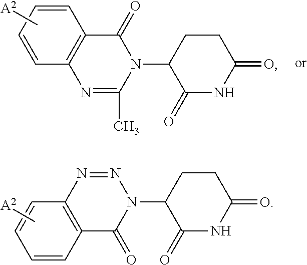

36. (canceled)

37. The compound of claim 1, or a pharmaceutically acceptable salt thereof, wherein the degradation moiety has the structure of Formula C: 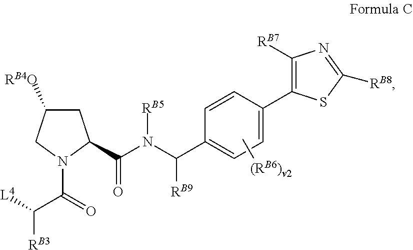
wherein
L⁴ is —N(R^{B1})(R^{B2}), 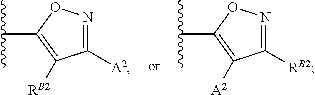
R^{B1} is H, A², optionally substituted C₁-C₆ alkyl, or optionally substituted C₁-C₆ heteroalkyl;
R^{B2} is H, optionally substituted C₁-C₆ alkyl, or optionally substituted C₁-C₆ heteroalkyl;
R^{B3} is A², optionally substituted C₁-C₆ alkyl, optionally substituted C₁-C₆ heteroalkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B4} is H, optionally substituted C₁-C₆ alkyl, optionally substituted C₃-C₁₀ carbocyclyl, optionally substituted C₆-C₁₀ aryl, optionally substituted C₁-C₆ alkyl C₃-C₁₀ carbocyclyl, or optionally substituted C₁-C₆ alkyl C₆-C₁₀ aryl;
R^{B5} is H, optionally substituted C₁-C₆ alkyl, or optionally substituted C₁-C₆ heteroalkyl;
v2 is 0, 1, 2, 3, or 4;
each R^{B6} is, independently, A², halogen, optionally substituted C₁-C₆ alkyl, optionally substituted C₁-C₆ heteroalkyl, optionally substituted C₃-C₁₀ carbocyclyl, optionally substituted C₂-C₉ heterocyclyl, optionally substituted C₆-C₁₀ aryl, optionally substituted C₂-C₉ heteroaryl, optionally substituted C₂-C₆ alkenyl, optionally substituted C₂-C₆ heteroalkenyl, hydroxy, thiol, or optionally substituted amino;
each of R^{B7} and R^{B8} is, independently, H, halogen, optionally substituted C₁-C₆ alkyl, or optionally substituted C₆-C₁₀ aryl;
R^{B9} is H or optionally substituted C₁-C₆ alkyl; and
A² is a bond between the degradation moiety and the linker;
wherein one and only one of R^{B1}, R^{B3}, and R^{B6} is A²,
or a pharmaceutically acceptable salt thereof.

38. The compound of claim 37, or a pharmaceutically acceptable salt thereof, wherein the degradation moiety has the structure of Formula C1:

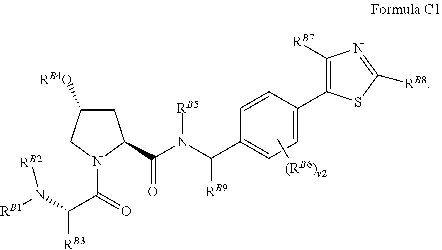

39. The compound of claim 37, or a pharmaceutically acceptable salt thereof, wherein the degradation moiety is

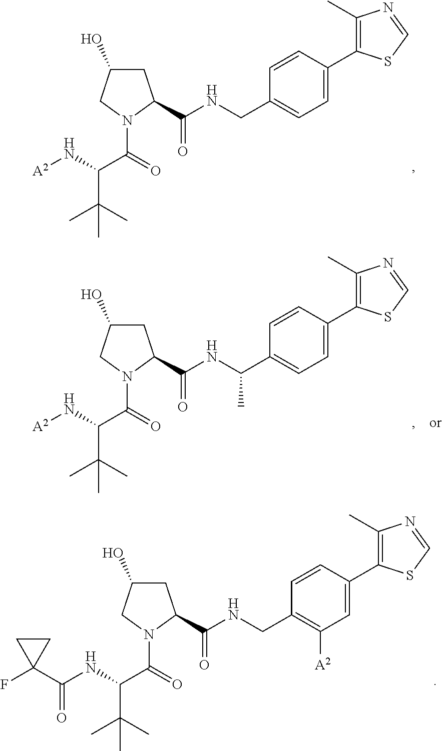

40.-41. (canceled)

42. The compound of claim 37, or a pharmaceutically acceptable salt thereof, wherein the degradation moiety has the structure of Formula C2:

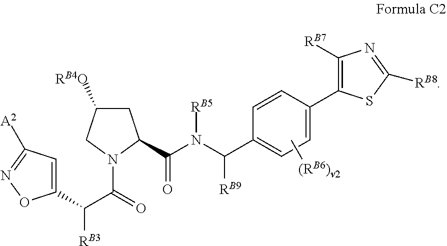

43. The compound of claim 37, or a pharmaceutically acceptable salt thereof, wherein R^{B9} is optionally substituted C₁-C₆ alkyl, and R^{B9} is bonded to (S)-stereogenic center.

44.-45. (canceled)

46. The compound of claim 37, or a pharmaceutically acceptable salt thereof, wherein the degradation moiety is

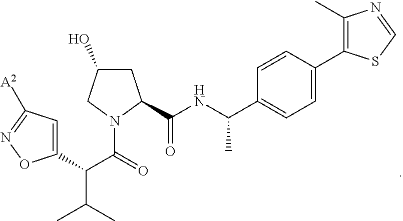

47. The compound of claim 1, or a pharmaceutically acceptable salt thereof, wherein the linker has the structure of Formula II:
A¹-(B¹)_{f}—(C¹)_{g}—(B²)ₕ-(D)-(B³)ᵢ—(C²)ⱼ—(B⁴)ₖ-A², Formula II
or a pharmaceutically acceptable salt thereof,
wherein
A¹ is a bond between the linker and ring system A;
A² is a bond between the degradation moiety and the linker;
each of B¹, B², B³, and B⁴ is, independently, optionally substituted C₁-C₄ alkyl, optionally substituted C₆-C₁₀ aryl, optionally substituted C₆-C₁₀ aryl C₁₋₄ alkyl, optionally substituted C₁-C₄ heteroalkyl, optionally substituted C₃-C₁₀ cycloalkyl, optionally substituted C₂-C₆ heterocyclyl, optionally substituted C₆₋₁₂ aryl, O, S, S(O)₂, or NR^{N};
each R^{N} is, independently, H, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₂₋₆ heteroaryl, or optionally substituted C₁₋₇ heteroalkyl;
each of C¹ and C² is, independently, carbonyl, thiocarbonyl, sulphonyl, or phosphoryl;
each of f, g, h, i, j, and k is, independently, 0 or 1; and
D is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₂₋₆ heteroaryl, optionally substituted C₆₋₁₂ aryl, optionally substituted C₂-C₁₀ polyethylene glycol, or optionally substituted C₁₋₁₀ heteroalkyl, or a chemical bond linking A¹-(B¹)_{f}—(C¹)_{g}—(B²)ₕ— to —(B³)ᵢ—(C₂)ⱼ—(B⁴)ₖ-A².

48. The compound of claim 47, or a pharmaceutically acceptable salt thereof, wherein each of B¹, B², B³, and B⁴ is, independently, optionally substituted C₁-C₂ alkyl, optionally substituted C₁-C₃ heteroalkyl, optionally substituted C₂-C₆ heterocyclyl, or NR^{N}.

49. The compound of claim 47, or a pharmaceutically acceptable salt thereof, wherein each R^{N} is, independently, H or optionally substituted C₁-C₄ alkyl.

50. (canceled)

51. The compound of claim 47, or a pharmaceutically acceptable salt thereof, wherein each of B¹ and B⁴ is independently,

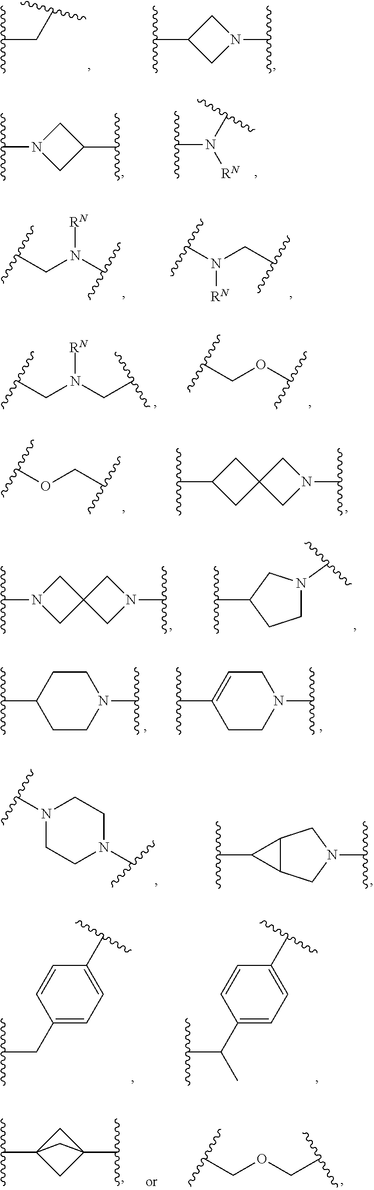

52. The compound of claim 51, or a pharmaceutically acceptable salt thereof, wherein B¹ is

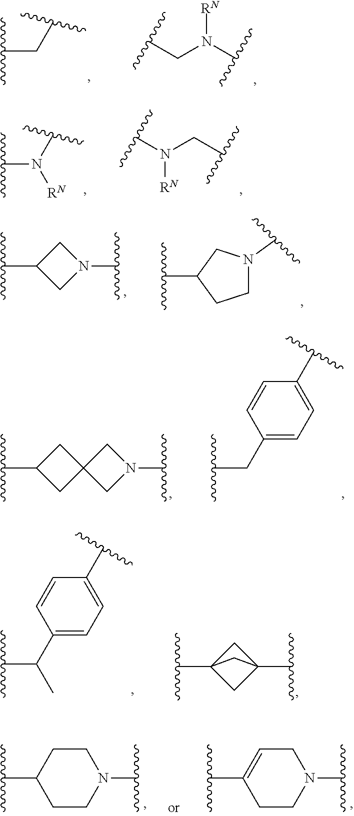

53. The compound of claim 51, or a pharmaceutically acceptable salt thereof, wherein B⁴ is

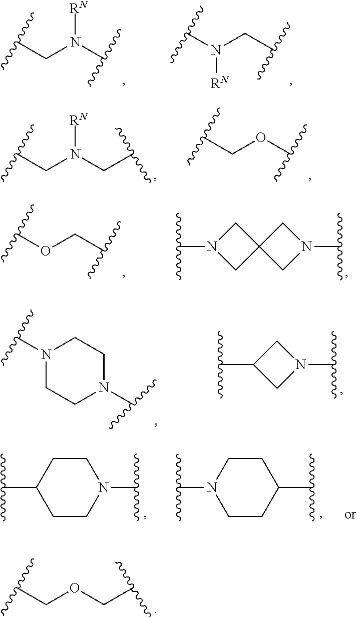

54. The compound of claim 51, or a pharmaceutically acceptable salt thereof, wherein each of C¹ and C² is independently

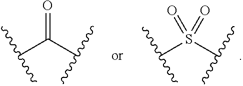

55.-58. (canceled)

59. The compound of claim 47, or a pharmaceutically acceptable salt thereof, wherein B² is

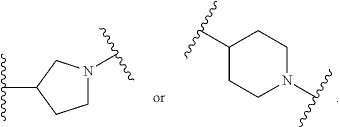 [SMILES: *C1CCN(*)C1 ; *C1CCN(*)CC1]

60. The compound of claim 47, or a pharmaceutically acceptable salt thereof, wherein D is optionally substituted C₁-C₁₀ alkyl.

61.-71. (canceled)

72. The compound of claim 47, or a pharmaceutically acceptable salt thereof, wherein the linker has the structure of

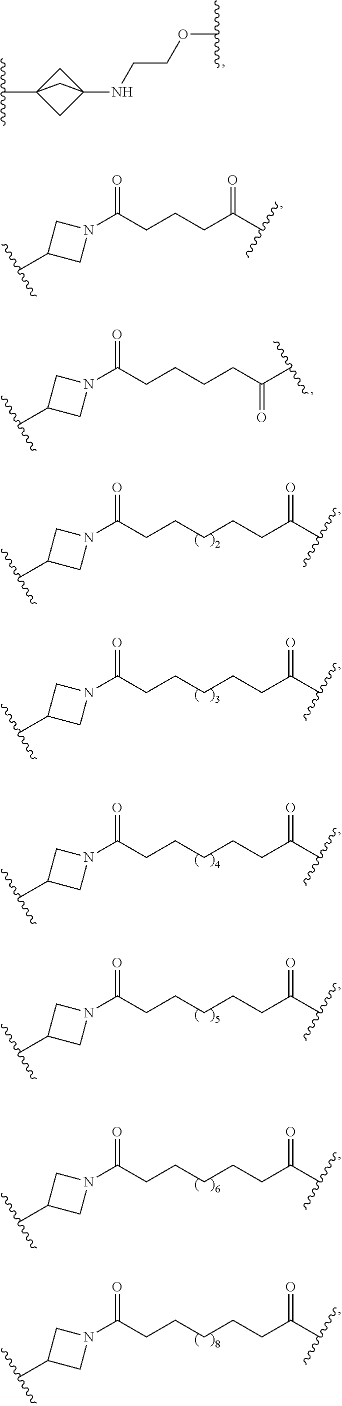

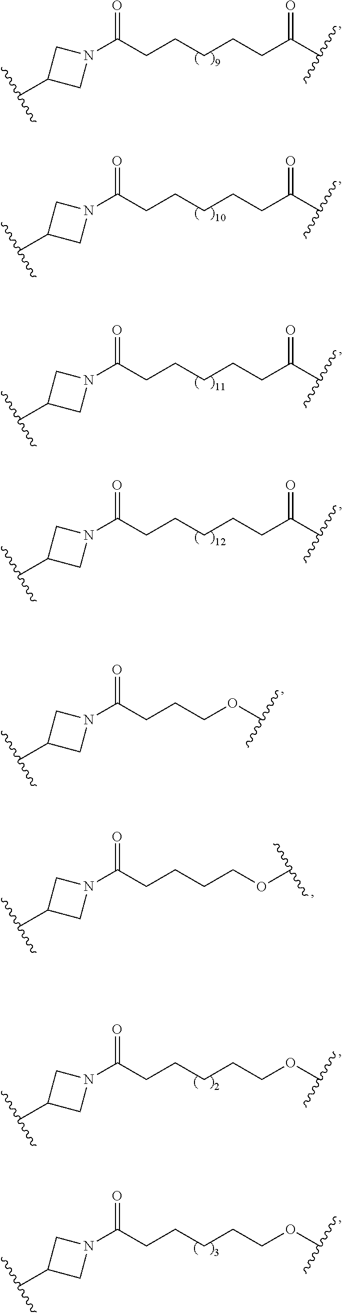

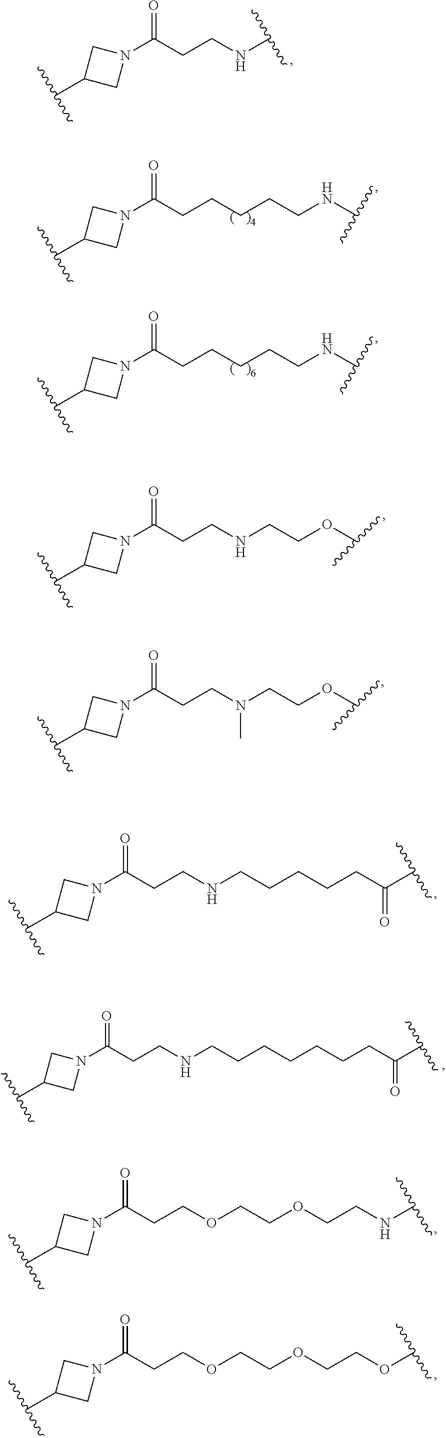

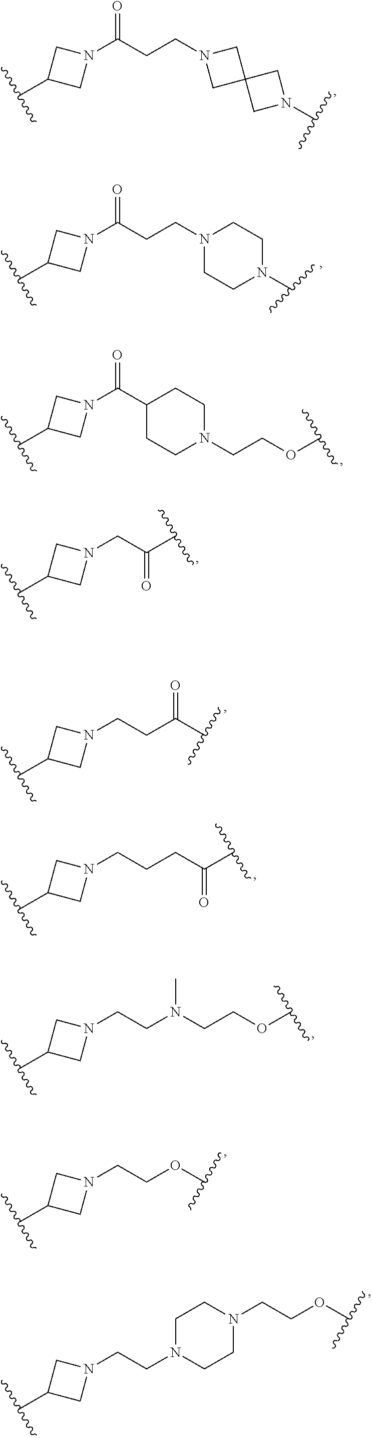

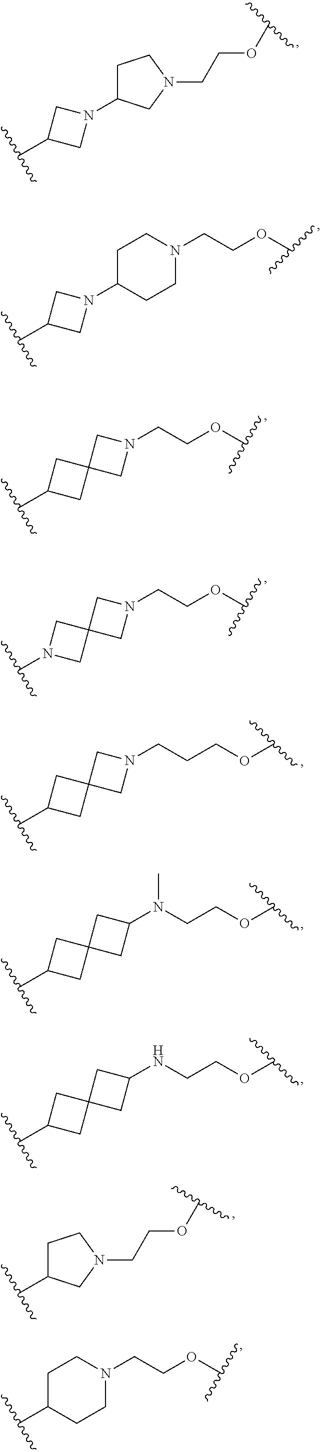

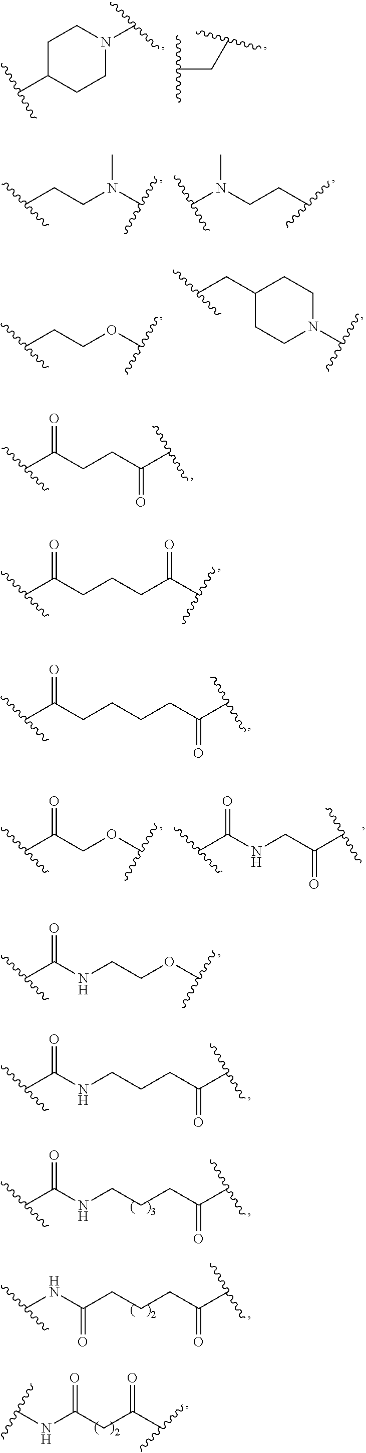

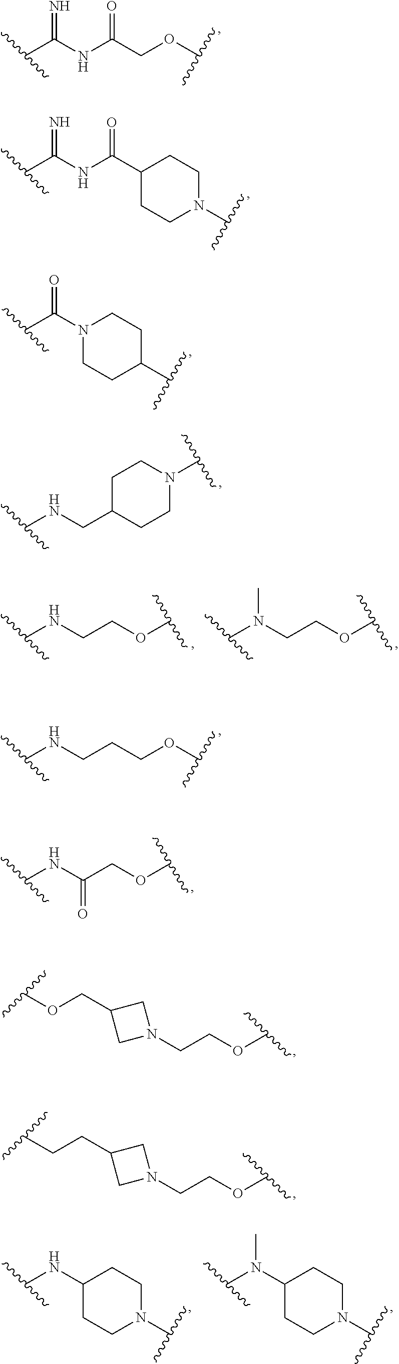

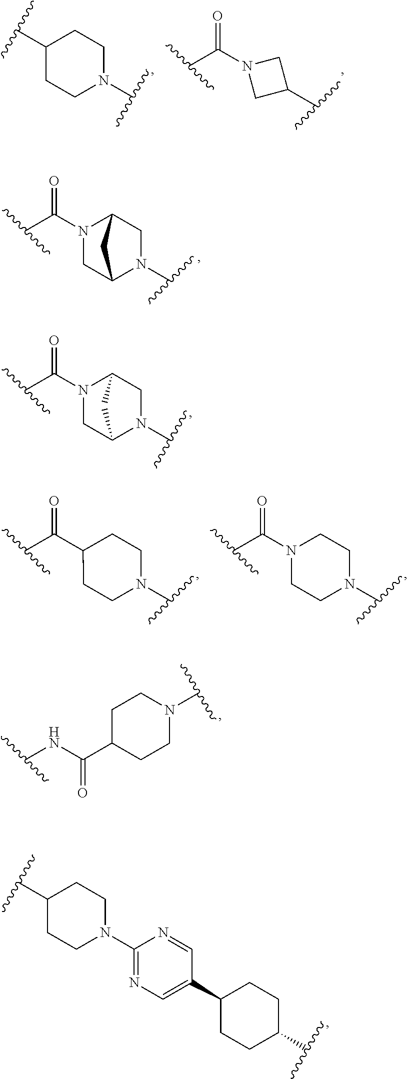

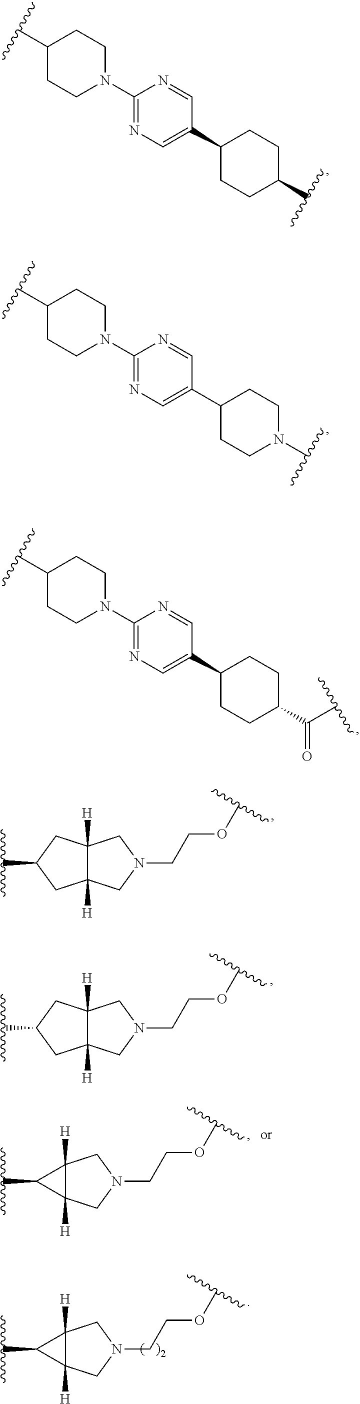

73.-74. (canceled)

75. The compound of claim 47, or a pharmaceutically acceptable salt thereof, wherein the linker has the structure of

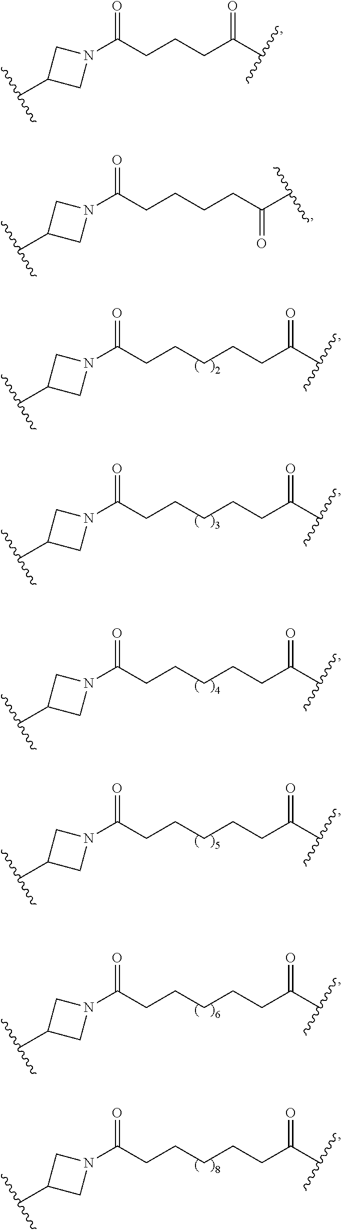

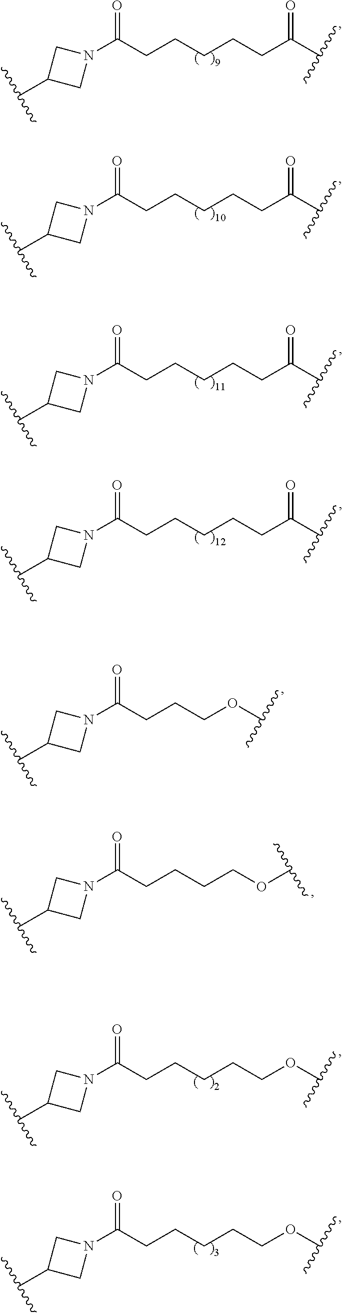

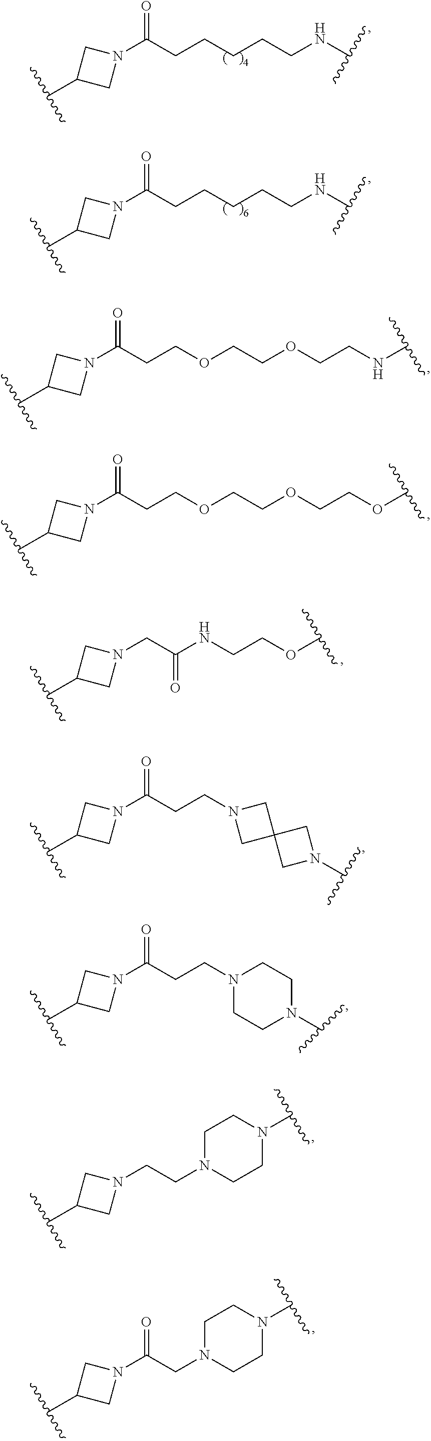

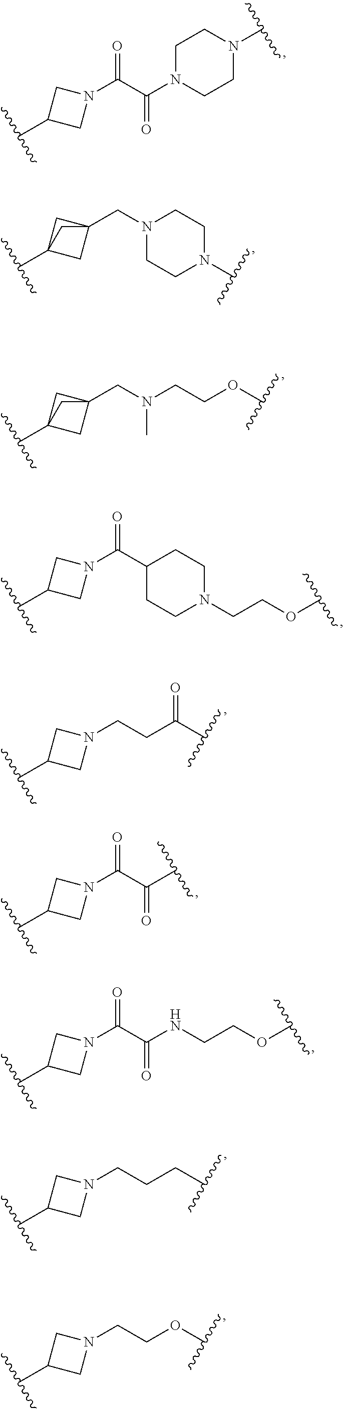

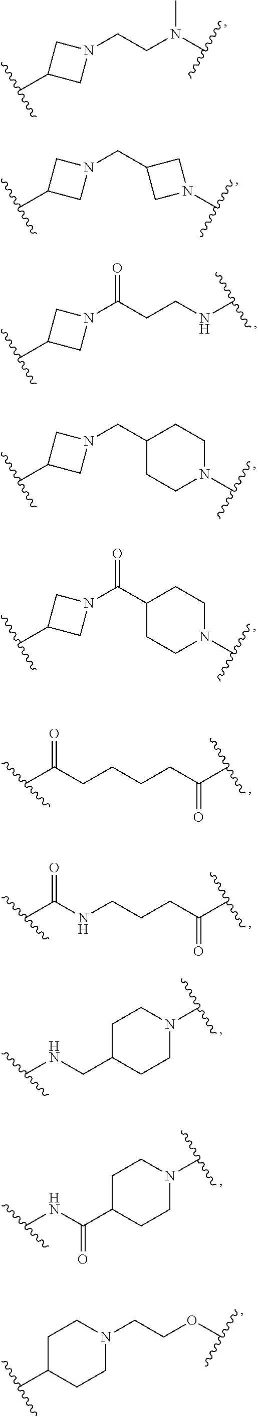

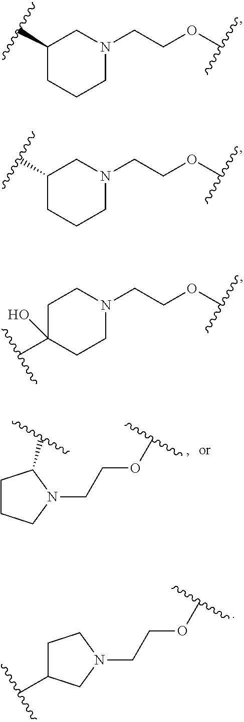 [SMILES: *OCCN1CCC[C@@H](*)C1 ; *OCCN1CCC[C@H](*)C1 ; *OCCN1CCC(*)(O)CC1 ; *OCCN1CCC[C@@H]1* ; *OCCN1CCC(*)C1]

76. A compound selected from the group consisting of I-310 in Table 1 and pharmaceutically acceptable salts thereof.

77.-79. (canceled)

80. The compound of claim 1, or a pharmaceutically acceptable salt thereof, wherein the compound has a ratio of BRG1 IC₅₀ to BRM IC₅₀ of at least 15.

81.-86. (canceled)

87. A method of treating a BAF complex-related disorder in a subject in need thereof, the method comprising administering to the subject an effective amount of a compound of claim 1, wherein the BAF complex-related disorder is cancer or a viral infection.

88.-90. (canceled)

91. A method of treating a disorder related to a BRG1 loss of function mutation in a subject in need thereof, the method comprising administering to the subject an effective amount of a compound of claim 1, wherein the disorder related to a BRG1 loss of function mutation is cancer.

92.-94. (canceled)

95. A method of treating cancer in a subject in need thereof, the method comprising administering to the subject an effective amount of a compound of claim 1.

96. The method of claim 95, wherein the cancer is non-small cell lung cancer, colorectal cancer, bladder cancer, cancer of unknown primary, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, small-cell lung cancer, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

97. The method of claim 95, wherein the cancer is non-small cell lung cancer, colorectal cancer, bladder cancer, cancer of unknown primary, glioma, breast cancer, melanoma, non-melanoma skin cancer, endometrial cancer, or penile cancer.

98. (canceled)

99. (canceled)

100. A method of treating a cancer selected from the group consisting of melanoma, prostate cancer, breast cancer, bone cancer, renal cell carcinoma, and a hematologic cancer in a subject in need thereof, the method comprising administering to the subject an effective amount of a compound claim 1.

101.-132. (canceled)
